(19) United States
(12) Patent Application Publication
(10) Pub. No.: US 2013/0317027 A1
(43) Pub. Date: Nov. 28, 2013

(54) COMPOUNDS AND THERAPEUTIC USES THEREOF

(71) Applicant: Myrexis, Inc, Salt Lake City, UT (US)

(72) Inventors: J. Adam Willardsen, Draper, UT (US); Jeffrey W. Lockman, Princeton Junction, NJ (US); Brett R. Murphy, Collbran, CO (US); Weston R. Judd, Farmington, UT (US); In Chul Kim, Louisville, KY (US); Se-Ho Kim, Salt Lake City, UT (US); Daniel Feodore Zigar, Salt Lake City, UT (US); Kraig M. Yager, Murray, UT (US); Tracey C. Fleischer, Sandy, UT (US); Ryan T. Terry-Lorenzo, Marlborough, MA (US); J. Jay Boniface, Salt Lake City, UT (US); Daniel P. Parker, Sandy, UT (US); Ian A. McAlexander, Salt Lake City, UT (US); Matthew Gregory Bursavich, Needham, MA (US); David M. Dastrup, Columbus, OH (US); Christophe Hoarau, Pleasant Hill, CA (US); David A. Gerrish, Lakewood, OH (US); Paul R. Sebahar, Sandy, UT (US); Dange Vijay Kumar, Salt Lake City, UT (US)

(73) Assignee: Myrexis, Inc., Salt Lake City, UT (US)

(21) Appl. No.: 13/708,235

(22) Filed: Dec. 7, 2012

Related U.S. Application Data
(63) Continuation-in-part of application No. 13/601,879, filed on Aug. 31, 2012.
Application No. 13/601,879 is a continuation of application No. PCT/US11/26752, filed on Mar. 1, 2011.
(60) Provisional application No. 61/380,083, filed on Sep. 3, 2010.
(60) Provisional application No. 61/360,364, filed on Jun. 30, 2010.
(60) Provisional application No. 61/309,342, filed on Mar. 1, 2010.
(60) Provisional application No. 61/568,778, filed on Dec. 9, 2011.

(51) Int. Cl. C07D 213/46; C07D 295/135; C07D 235/06; C07D 401/12

(52) U.S. Cl. CPC C07D 213/46; C07D 401/12; C07D 295/135; C07D 235/06
USPC 514/235.5; 546/332; 514/357; 546/261; 514/335; 546/275.4; 514/341; 546/194; 514/318; 546/265; 514/332; 548/304.4; 514/394; 546/276.4; 514/343; 546/272.7; 544/284; 514/266.21; 544/360; 435/375; 514/253.01; 544/131

(57) ABSTRACT

The invention relates to compounds, pharmaceutical compositions and methods useful for treating cancer, systemic or chronic inflammation, rheumatoid arthritis, diabetes, obesity, T-cell mediated autoimmune disease, ischemia, and other complications associated with these diseases and disorders.

### CROSS-REFERENCE TO RELATED APPLICATIONS

This application is a continuation-in-part of prior U.S. application Ser. No. 13/601,879, filed Aug. 31, 2012; which is a continuation of prior International Application No. PCT/US11/26752, filed Mar. 1, 2011, and published as WO 2011/109441; which claims the benefit of U.S. Provisional Application No. 61/380,083, filed Sep. 3, 2010, U.S. Provisional Application No. 61/360,364, filed Jun. 30, 2010, and U.S. Provisional Application No. 61/309,342, filed Mar. 1, 2010; this application also claims the benefit of U.S. Provisional Application No. 61/568,778, filed Dec. 9, 2011; the contents of all of the afore-listed applications are hereby incorporated by reference herein in their entirety.

### FIELD OF THE INVENTION

The present invention relates generally to the field of medicinal chemistry. Specifically, the present invention provides compounds that inhibit Nicotinamide phosphoribosyltransferase (Nampt). The invention also provides methods for making these compounds, pharmaceutical compositions comprising these compounds, and methods for treating diseases with these compounds; particularly cancer, systemic or chronic inflammation, rheumatoid arthritis, diabetes, obesity, T-cell mediated autoimmune disease, ischemia, and other complications associated with these diseases and disorders, that respond favorably to the inhibition of Nampt.

### BACKGROUND OF THE INVENTION

Nicotinamide phosphoribosyltransferase (Nampt; also know as visfatin and pre-B-cell colony-enhancing factor 1 (PBEF)) catalyzes the condensation of nicotinamide (NaM) with 5-phosphoribosyl-1-pyrophosphate to yield nicotinamide mononucleotide. This is the first and rate-limiting step in one biosynthetic pathway that cells use to make nicotinamide adenine dinucleotide (NAD⁺).

NAD⁺ has many important cellular functions. Classically, it plays a role as a key coenzyme in metabolic pathways, where it continually cycles between its oxidized form (NAD⁺) and its reduced form (NADH). More recently, NAD⁺ has been shown to be involved in genome integrity maintainence, stress response, and Ca²⁺ signaling, where it is consumed by enzymes including poly(ADP-ribose) polymerases (PARPs), sirtuins, and cADP-ribose synthases, respectively. (Reviewed in Belenky, P. et al., NAD⁺ metabolism in health and disease. Trends Biochem. Sci. 32, 12-19 (2007).)

As a critical coenzyme in redox reactions, NAD⁺ is required in glycolysis and the citric acid cycle; where it accepts the high energy electrons produced and, as NADH, passes these electrons on to the electron transport chain. The NADH-mediated supply of high energy electrons is the driving force behind oxidative phosphorylation, the process by which the majority of ATP is generated in aerobic cells. Consequently, having sufficient levels of NAD⁺ available in the cell is critical for the maintenance of proper ATP levels in the cell. Understandably, reduction in cellular NAD⁺ levels by Nampt inhibition can be expected to eventually lead to depletion of ATP and, ultimately, cell death.

In view of the above, it is perhaps not surprising that inhibitors of Nampt are being developed as chemotherapeutic agents for the treatment of cancer. In fact, there are currently two Nampt inhibitors in clinical trials for the treatment of cancer (Holen, K. et al. The pharmacokinetics, toxicities, and biologic effects of FK866, a nicotinamide adenine dinucleotide biosynthesis inhibitor. Invest. New Drugs. 26, 45-51 (2008); Hovstadius, P. et al. A Phase I study of CHS 828 in patients with solid tumor malignancy. Clin. Cancer Res. 8, 2843-2850 (2002); Ravaud, A. et al., Phase I study and pharmacokinetic of CHS-828, a guanidino-containing compound, administered orally as a single dose every 3 weeks in solid tumours: an ECSG/EORTC study. Eur. J. Cancer. 41, 702-707 (2005); and von Heideman, A. et al. Safety and efficacy of NAD depleting cancer drugs: results of a phase I clinical trial of CHS 828 and overview of published data. Cancer Chemother. Pharmacol. (2009) Sep. 30 [Epub ahead of print]).

Consequently, there is a clear need for compounds that inhibit Nampt, which can not only be used in the treatment of cancer, but can also be used in the treatment of systemic or chronic inflammation, rheumatoid arthritis, diabetes, obesity, T-cell mediated autoimmune disease, ischemia, and other complications associated with these diseases and disorders.

### BRIEF SUMMARY OF THE INVENTION

The present invention provides chemical compounds that inhibit the activity of Nampt. These compounds can be used in the treatment of cancer, systemic or chronic inflammation, rheumatoid arthritis, diabetes, obesity, T-cell mediated autoimmune disease, ischemia, and other complications associated with these diseases and disorders.

Specifically, the present invention provides compounds of Formula I

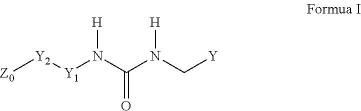

and pharmaceutically acceptable salts and solvates thereof;
wherein Y, Y₁, Y₂, and Z₀ are as defined herein below.

The present invention further provides compounds of Formula II

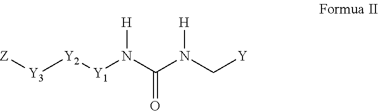

and pharmaceutically acceptable salts and solvates thereof;
wherein Y, Y₁, Y₂, Y₃, and Z are as defined herein below.

The present invention further provides compounds of Formula III

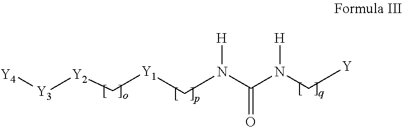

and pharmaceutically acceptable salts and solvates thereof;
wherein Y, Y₁, Y₂, Y₃, and Y₄ are as defined herein below.

The present invention further provides compounds of Formula IV

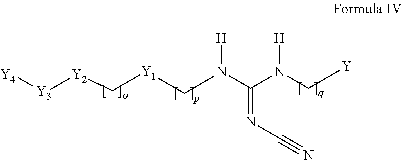

and pharmaceutically acceptable salts and solvates thereof;
wherein o, p, q, Y, Y₁, Y₂, Y₃, and Y₄ are as defined herein below.

As noted above, the present invention provides chemical compounds that inhibit the activity of Nampt, and therefore can be used in the treatment of cancer, systemic or chronic inflammation, rheumatoid arthritis, diabetes, obesity, T-cell mediated autoimmune disease, ischemia, and other complications associated with these diseases and disorders. Thus, in a related aspect, the present invention also provides methods for treating cancer, systemic or chronic inflammation, rheumatoid arthritis, diabetes, obesity, T-cell mediated autoimmune disease, ischemia, and other complications associated with these diseases and disorders, by administering to a patient in need of such treatment a therapeutically effective amount of one or more of the compounds of the present invention.

Also provided is the use of the compounds of the present invention for the manufacture of a medicament useful for therapy, particularly for the treatment of cancer, systemic or chronic inflammation, rheumatoid arthritis, diabetes, obesity, T-cell mediated autoimmune disease, ischemia, and other complications associated with these diseases and disorders. In addition, the present invention also provides a pharmaceutical composition having one or more of the compounds of the present invention and one or more pharmaceutically acceptable excipients. Further, methods for the treatment of cancer, systemic or chronic inflammation, rheumatoid arthritis, diabetes, obesity, T-cell mediated autoimmune disease, ischemia, and other complications associated with these diseases and disorders, by administering to a patient in need of such treatment, a pharmaceutical composition of the present invention, is also encompassed.

In addition, the present invention further provides methods for treating or delaying the onset of the symptoms associated with cancer, systemic or chronic inflammation, rheumatoid arthritis, type 2 diabetes, obesity, T-cell mediated autoimmune disease, ischemia, and other complications associated with these diseases and disorders. These methods comprise administering an effective amount of one or more of the compounds of the present invention, preferably in the form of a pharmaceutical composition or medicament, to an individual having, or at risk of developing, cancer, systemic or chronic inflammation, rheumatoid arthritis, type 2 diabetes, obesity, T-cell mediated autoimmune disease, ischemia, and other complications associated with these diseases and disorders.

The compounds of the present invention can be used in combination therapies. Thus, combination therapy methods are also provided for treating or delaying the onset of the symptoms associated with cancer, systemic or chronic inflammation, rheumatoid arthritis, type 2 diabetes, obesity, T-cell mediated autoimmune disease, ischemia, and other complications associated with these diseases and disorders. Such methods comprise administering to a patient in need thereof one or more of the compounds of the present invention and, together or separately, at least one other anti-cancer, anti-inflammation, anti-rheumatoid arthritis, anti-type 2 diabetes, anti-obesity, anti-T-cell mediated autoimmune disease, or anti-ischemia therapy.

The foregoing and other advantages and features of the embodiments of the present invention, and the manner in which they are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only, and are not intended to be limiting.

Other features and advantages of the invention will be apparent to one of skill in the art from the following detailed description, and from the claims below.

### BRIEF DESCRIPTION OF THE DRAWINGS

FIG. 1(A) depicts how the activities of Nampt and PARP are interconnected via their differential actions in the NAD⁺/NaM cycle; FIG. 1(B) illustrates how PARP activation in BRCA-proficient cells by certain types of DNA damage causes NAD⁺ conversion into nicotinamide (NaM) thereby requiring Nampt activity for NAD⁺ salvage; FIG. 1 (C) depicts how, in BRCA-deficient cells that require PARP for life, PARP inhibitors and Nampt inhibitors can synergize to cause cell death.

### DETAILED DESCRIPTION OF THE INVENTION

### 1. Definitions

As used herein, the term “alkyl” as employed herein by itself or as part of another group refers to a saturated aliphatic hydrocarbon straight chain or branched chain group having, unless otherwise specified, 1 to 20 carbon atoms (whenever it appears herein, a numerical range such as “1 to 20” refers to each integer in the given range; e.g., “1 to 20 carbon atoms” means that the alkyl group can consist of 1, 2 or 3 carbon atoms, or more carbon atoms, up to a total of 20). An alkyl group can be in an unsubstituted form or substituted form with one or more substituents (generally one to three substitutents can be present except in the case of halogen substituents, e.g., perchloro). For example, a C₁₋₆alkyl group refers to a straight or branched aliphatic group containing 1 to 6 carbon atoms (e.g., include methyl, ethyl, propyl, isopropyl, butyl, sec-butyl, tert-butyl, 3-pentyl, hexyl, etc.), which can be optionally substituted.

As used herein, “lower alkyl” refers to an alkyl group having from 1 to 6 carbon atoms.

The term “alkylene” as used herein means a saturated aliphatic hydrocarbon straight chain or branched chain group having from 1 to 20 carbon atoms having two connecting points (i.e., a “divalent” chain). For example, “ethylene” represents the group —CH₂—CH₂— and “methylene” represents the group —CH₂—. Alkylene chain groups can also be thought of as multiple methylene groups. For example, ethylene contains two methylene groups. Alkylene groups can also be in an unsubstituted form or substituted form with one or more substituents.

The term “alkenyl” as employed herein by itself or as part of another group means a straight or branched divalent chain radical of 2-10 carbon atoms (unless the chain length is otherwise specified), including at least one double bond between two of the carbon atoms in the chain. The alkenyl group can also be in an unsubstituted form or substituted form with one or more substituents (generally one to three substitutents except in the case of halogen substituents, e.g., perchloro or perfluoroalkyls). For example, a C₂₋₆alkenyl group refers to a straight or branched chain radical containing 2 to 6 carbon atoms and having at least one double bond between two of the carbon atoms in the chain (e.g., ethenyl, 1-propenyl, 2-propenyl, 2-methyl-1-propenyl, 1-butenyl and 2-butenyl, which can be optionally substituted).

The term “alkenylene” as used herein means an alkenyl group having two connecting points. For example, “ethenylene” represents the group —CH═CH—. Alkenylene groups can also be in an unsubstituted form or substituted form with one or more substituents.

The term “alkynyl” as used herein by itself or as part of another group means a straight or branched chain radical of 2-10 carbon atoms (unless the chain length is otherwise specified), wherein at least one triple bond occurs between two of the carbon atoms in the chain. The alkynyl group can be in an unsubstituted form or substituted form with one or more substituents (generally one to three substitutents except in the case of halogen substituents, e.g., perchloro or perfluoroalkyls). For example, a C₂₋₆alkynyl group refers to a straight or branched chain radical containing 2 to 6 carbon atoms, which can be optionally substituted, and having at least one triple bond between two of the carbon atoms in the chain (e.g., ethynyl, 1-propynyl, 1-methyl-2-propynyl, 2-propynyl, 1-butynyl and 2-butynyl).

The term “alkynylene” as used herein means an alkynyl having two connecting points. For example, “ethynylene” represents the group —C≡C—. Alkynylene groups can also be in an unsubstituted form or substituted form with one or more substituents.

The term “carbocycle” as used herein by itself or as part of another group means cycloalkyl and non-aromatic partially saturated carbocyclic groups such as cycloalkenyl and cycloalkynyl. A carbocycle can be in an unsubstituted form or substituted form with one or more substituents so long as the resulting compound is sufficiently stable and suitable for use in the embodiments of the present invention.

The term “cycloalkyl” as used herein by itself or as part of another group refers to a fully saturated 3- to 8-membered cyclic hydrocarbon ring (i.e., a cyclic form of an alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with other such rings) (“polycyclic cycloalkyl”). Thus, a cycloalkyl can exist as a monocyclic ring, bicyclic ring, or a spiral ring. When a cycloalkyl is referred to as a Cₓ cycloalkyl, this means a cycloalkyl in which the fully saturated cyclic hydrocarbon ring (which may or may not be fused to another ring) has x number of carbon atoms. When a cycloalkyl is recited as a substituent on a chemical entity, it is intended that the cycloalkyl moiety is attached to the entity through a single carbon atom within the fully saturated cyclic hydrocarbon ring of the cycloalkyl. In contrast, a substituent on a cycloalkyl can be attached to any carbon atom of the cycloalkyl. A cycloalkyl group can be unsubstituted or substituted with one or more substitutents so long as the resulting compound is sufficiently stable and suitable for use in the embodiments of the present invention. Examples of cycloalkyl groups include, for example, cyclopropyl, cyclobutyl, cyclopentyl, cyclohexyl and cycloheptyl.

The term “cycloalkenyl” as used herein by itself or as part of another group refers to a non-aromatic partially saturated 3- to 8-membered cyclic hydrocarbon ring having a double bond therein (i.e., a cyclic form of an alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl can exist as a monocyclic ring, bicyclic ring, polycyclic or a spiral ring. When a cycloalkenyl is referred to as a Cₓ cycloalkenyl, this means a cycloalkenyl in which the non-aromatic partially saturated cyclic hydrocarbon ring (which may or may not be fused to another ring) has x number of carbon atoms. When a cycloalkenyl is recited as a substituent on a chemical entity, it is intended that the cycloalkenyl moiety is attached to the entity through a carbon atom within the non-aromatic partially saturated ring (having a double bond therein) of the cycloalkenyl. In contrast, a substituent on a cycloalkenyl can be attached to any carbon atom of the cycloalkenyl. A cycloalkenyl group can be in an unsubstituted form or substituted form with one or more substitutents. Examples of cycloalkenyl groups include cyclopentenyl, cycloheptenyl and cyclooctenyl.

The term “heterocycle” (or “heterocyclyl” or “heterocyclic” or “heterocyclo”) as used herein by itself or as part of another group means a saturated or partially saturated 3-7 membered non-aromatic cyclic ring formed with carbon atoms and from one to four heteroatoms independently selected from the group consisting of O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of atoms with such other rings) (“polycyclic heterocycle”). Thus, a heterocycle can exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle can be in an unsubstituted form or substituted form with one or more substituents so long as the resulting compound is sufficiently stable and suitable for use in the embodiments of the present invention.

Some examples of saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with up to 7 carbon atoms in the ring (“monocylic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is referred to as a Cₓ aryl, this means an aryl in which the all-carbon aromatic ring (which may or may not be fused to another ring) has x number of carbon atoms. When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl can be in an unsubstituted form or substituted form with one or more substituents so long as the resulting compound is sufficiently stable and suitable for use in the embodiments of the present invention.

The term “heteroaryl” as employed herein refers to a stable aromatic ring having up to 7 ring atoms with 1, 2, 3 or 4 hetero ring actoms in the ring which are oxygen, nitrogen or sulfur or a combination thereof (“monocylic heteroaryl”). In addition to monocyclic hetero-aromatic rings, the term “heteroaryl” also encompasses a group having the monocyclic hetero-aromatic ring above fused to another cycloalkyl, cycloalkynyl, cycloalkenyl, heterocycle, aryl or heteroaryl ring (i.e., sharing an adjacent pair of atoms with such other rings) (“polycyclic heteroaryl”). When a heteroaryl is recited as a substituent on a chemical entity, it is intended that the heteroaryl moiety is attached to the entity through an atom within the heteroaromatic ring of the heteroaryl. In contrast, a substituent on a heteroaryl can be attached to any suitable atom of the heteroaryl. A heteroaryl can be in an unsubstituted form or substituted form with one or more substituents so long as the resulting compound is sufficiently stable and suitable for use in the embodiments of the present invention.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom can be in the form of an N-oxide, e.g., a pyridyl N-oxide, pyrazinyl N-oxide and pyrimidinyl N-oxide.

As used herein, the term “halo” refers to chloro, fluoro, bromo, or iodo substitutents.

As used herein, the term “hydro” refers to a bound hydrogen atom (—H group).

As used herein, the term “hydroxyl” refers to an —OH group.

As used herein, the term “alkoxy” refers to an —O—(C₁₋₁₂ alkyl). Lower alkoxy refers to —O-(lower alkyl) groups.

As used herein, the term “alkynyloxy” refers to an —O—(C₂₋₁₂ alkynyl).

As used herein, the term “cycloalkyloxy” refers to an —O-cycloalkyl group.

As used herein, the term “heterocycloxy” refers to an —O-heterocycle group.

As used herein, the term “aryloxy” refers to an —O-aryl group. Examples of aryloxy groups include, but are not limited to, phenoxy and 4-methylphenoxy.

The term “heteroaryloxy” refers to an —O-heteroaryl group.

The terms “arylalkoxy” and “heteroarylalkoxy” are used herein to mean alkoxy group substituted with an aryl group and a heteroaryl group, respectively. Examples of arylalkoxy groups include, but are not limited to, benzyloxy and phenethyloxy.

As used herein, the term “mercapto” or “thiol” group refers to an —SH group.

The term “alkylthio” group refers to an —S-alkyl group.

The term “arylthio” group refers to an —S-aryl group.

The term “arylalkyl” is used herein to mean above-defined alkyl group substituted by an aryl group defined above. Examples of arylalkyl groups include benzyl, phenethyl and naphthylmethyl, etc. An arylalkyl group can be unsubstituted or substituted with one or more substituents so long as the resulting compound is sufficiently stable and suitable for use in the embodiments of the present invention.

The term “heteroarylalkyl” is used herein to mean an alkyl group, as defined above, substituted by any heteroaryl group. A heteroarylalkyl can be unsubstituted or substituted with one or more substituents, so long as the resulting compound is sufficiently stable and suitable for use in the embodiments of the present invention.

The term “heteroarylalkenyl” is used herein to mean any of the above-defined alkenyl groups substituted by any of the above-defined heteroaryl groups.

The term “arylalkynyl” is used herein to mean any of the above-defined alkynyl groups substituted by any of the above-defined aryl groups.

The term “heteroarylalkenyl” is used herein to mean any of the above-defined alkenyl groups substituted by any of the above-defined heteroaryl groups.

The term “arylalkoxy” is used herein to mean alkoxy group substituted by an aryl group as defined above.

“Heteroarylalkoxy” is used herein to mean any of the above-defined alkoxy groups substituted by any of the above-defined heteroaryl groups.

“Haloalkyl” means an alkyl group that is substituted with one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has an oxygen doubly-bonded to it; i.e. one of the ring carbon atoms is a —C(═O) group.

As used herein, the term “thiocarbonyl” group refers to a —C(═S)R″ group, with R″ as defined herein.

“Alkanoyl” refers to an —C(═O)-alkyl group.

The term “heterocyclonoyl” group refers to a heterocyclo group linked to the alkyl chain of an alkanoyl group.

The term “acetyl” group refers to a —C(═O)CH₃ group.

“Alkylthiocarbonyl” refers to an —C(═S)-alkyl group.

The term “cycloketone” refers to a carbocycle or heterocycle group in which one of the carbon atoms which form the ring has an oxygen doubly-bonded to it; i.e., one of the ring carbon atoms is a —C(═O) group.

The term “O-carboxy” group refers to a —OC(═O)R″ group, where R″ is as defined herein.

The term “C-carboxy” group refers to a —C(═O)OR″ groups where R″ is as defined herein.

As used herein, the term “carboxylic acid” refers to a C-carboxy group in which R″ is hydro. In other words, the term “carboxylic acid” refers to —COOH.

As used herein, the term “ester” is a C-carboxy group, as defined herein, wherein R″ is as defined above, except that it is not hydro (e.g., it is methyl, ethyl, or lower alkyl).

As used herein, the term “C-carboxy salt” refers to a —C(═O)O⁻M⁺ group wherein M⁺ is selected from the group consisting of lithium, sodium, magnesium, calcium, potassium, barium, iron, zinc and quaternary ammonium.

The term “carboxyalkyl” refers to —C₁₋₆alkylene-C(═O)OR″ (that is, a C₁₋₆ alkyl group connected to the main structure wherein the alkyl group is substituted with —C(═O)OR″ with R″ being defined herein). Examples of carboxyalkyl include, but are not limited to, —CH₂COOH, —(CH₂)₂COOH, —(CH₂)₃COOH, —(CH₂)₄COOH, and —(CH₂)₅COOH.

“Carboxyalkenyl” refers to -alkenylene-C(═O)OR″ with R″ being defined herein.

The term “carboxyalkyl salt” refers to a —(CH₂)ᵣC(═O)O-M⁺ wherein M⁺ is selected from the group consisting of lithium, sodium, potassium, calcium, magnesium, barium, iron, zinc and quaternary ammonium, and wherein r is 1-6.

The term “carboxyalkoxy” refers to —O—(CH₂)ᵣC(═O)OR″ wherein r is 1-6, and R″ is as defined herein.

“Cₓ carboxyalkanoyl” means a carbonyl group (—(O═)C—) attached to an alkyl or cycloalkylalkyl group that is substituted with a carboxylic acid or carboxyalkyl group, wherein the total number of carbon atom is x (an integer of 2 or greater).

“Cₓ carboxyalkenoyl” means a carbonyl group (—(O═)C—) attached to an alkenyl or alkyl or cycloalkylalkyl group that is substituted with a carboxylic acid or carboxyalkyl or carboxyalkenyl group, wherein at least one double bond (—CH═CH—) is present and wherein the total number of carbon atom is x (an integer of 2 or greater).

“Carboxyalkoxyalkanoyl” means refers to R″OC(═O)—C₁₋₆ alkylene-O—C₁₋₆ alkylene-C(═O)—, R″ is as defined herein.

“Amino” refers to an —NR^{x}R^{y} group, with R^{x} and R^{y} as defined herein.

“Alkylamino” means an amino group with a substituent being a C₁₋₆ alkyl.

“Aminoalkyl” means an alkyl group connected to the main structure of a molecule where the alkyl group has a substituent being amino.

“Quaternary ammonium” refers to a —⁺N(R^{x})(R^{y})(R^{z}) group wherein R^{x}, R^{y}, and R^{z} are as defined herein.

The term “nitro” refers to a —NO₂ group.

The term “O-carbamyl” refers to a —OC(═O)N(R^{x})(R^{y}) group with R^{x} and R^{y} as defined herein.

The term “N-carbamyl” refers to a R^{y}C(═O)N(R^{X})— group, with R^{x} and R^{y} as defined herein.

The term “O-thiocarbamyl” refers to a —OC(═S)N(R^{x})(R^{y}) group with R^{x} and R^{y} as defined herein.

The term “N-thiocarbamyl” refers to a R^{x}C(═S)NR^{y}— group, with R^{x} and R^{y} as defined herein.

“C-amido” refers to a —C(═O)N(R^{x})(R^{y}) group with R^{x} and R^{y} as defined herein.

“N-amido” refers to a R^{x}C(═O)N(R^{y})— group with R^{x} and R^{y} as defined herein.

“Aminothiocarbonyl” refers to a —C(═S)N(R^{x})(R^{y}) group with R^{x} and R^{y} as defined herein.

“Hydroxyaminocarbonyl” means a —C(═O)N(R^{x})(OH) group with R^{x} as defined herein.

“Alkoxyaminocarbonyl” means a —C(═O)N(R)(alkoxy) group with R^{x} as defined herein.

The terms “cyano” and “cyanyl” refer to a —C—N group.

The term “nitrile” group, as used herein, refers to a —C—N substituent.

The term “cyanato” refers to a —CNO group.

The term “isocyanato” refers to a —NCO group.

The term “thiocyanato” refers to a —CNS group.

The term “isothiocyanato” refers to a —NCS group.

The term “oxo” refers to a —C(═O)— group.

The term “sulfinyl” refers to a —S(═O)R″ group, where R″ is as defined herein.

The term “sulfonyl” refers to a —S(═O)₂R″ group, where R″ is as defined herein.

The term “sulfonamide” refers to a —(R^{x})N—S(═O)₂R″ group, with R″ and R^{x} as defined herein.

“Aminosulfonyl” means (R^{x})(R^{y})N—S(═O)₂— with R^{x} and R^{y} as defined herein.

“Aminosulfonyloxy” means a (R^{x})(R^{y})N—S(═O)₂—O— group with R^{x} and R^{y} as defined herein.

“Sulfonamidecarbonyl” means R″—S(═O)₂—N(R^{X})—C(═O)— with R″ and R^{x} as defined herein.

“Alkanoylaminosulfonyl” refers to an alkyl-C(═O)—N(R^{x})—S(═O)₂— group with R^{x} as defined herein.

The term “trihalomethylsulfonyl” refers to a X₃CS(═O)₂— group with X being halo.

The term “trihalomethylsulfonamide” refers to a X₃CS(═O)₂N(R^{x})— group with X being halo and R^{x} as defined herein.

R″ is selected from the group consisting of hydro, alkyl, cycloalkyl, aryl, heteroaryl and heterocycle, each being optionally substituted.

R^{x}, R^{y}, and R^{z} are independently selected from the group consisting of hydro and optionally substituted alkyl.

The term “methylenedioxy” refers to a —OCH₂O— group wherein the oxygen atoms are bonded to adjacent ring carbon atoms.

The term “ethylenedioxy” refers to a —OCH₂CH₂O— group wherein the oxygen atoms are bonded to adjacent ring carbon atoms.

As used herein, the phrase “optionally substituted” means substituted or unsubstituted.

Unless specifically stated otherwise or indicated by a bond symbol (dash, double dash, or triple dash), the connecting point to a recited group will be on the right-most stated group. Thus, for example, a hydroxyalkyl group is connected to the main structure through the alkyl and the hydroxyl is a substituent on the alkyl.

### 2. Therapeutic Compounds

The present invention provides chemical compounds that selectively inhibit the activity of Nampt. These compounds can be used in the treatment of cancer, systemic or chronic inflammation, rheumatoid arthritis, diabetes, obesity, T-cell mediated autoimmune disease, ischemia, and other complications associated with these diseases and disorders.

Specifically, the present invention provides compounds of Formula I

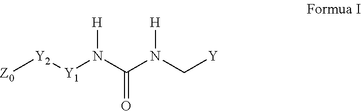

and pharmaceutically acceptable salts and solvates thereof;
wherein:

Y is phenyl, 2-pyridinyl, 3-pyridinyl, or 4-pyridinyl, wherein any ring carbon is optionally independently substituted with halo, C₁₋₅alkyl, nitro, cyano, C₁₋₅ alkoxy, C-amido, N-amido, C-carboxy, O-carboxy, sulfonamide, amino, hydroxyl, mercapto, alkylthio, sulfonyl, or sulfinyl;

Y₁ is divalent carbocycle, divalent heterocycle, divalent phenyl or divalent heteroaryl, wherein any ring atom is optionally independently substituted with halo, C₁₋₅ alkyl, nitro, cyano, trihalomethyl, C₁₋₅alkoxy, C-amido, N-amido, sulfonamide, amino, aminosulfonyl, hydroxyl, mercapto, alkylthio, sulfonyl, or sulfinyl, or

Y₁ is C₂₋₈alkylene or C₂₋₈alkenylene, optionally interrupted one, two, or three times by —O—, —S—, —S(═O)—, —S(═O)₂—, —OC(═O)N(R)—, —N(R)C(═O)O—, —C(═O)N(R)—, —N(R)C(═O)—, —N(R)C(═O)N(R)—, —N(R)—, —C(═O)—, —OC(═O)—, —C(═O)O—, —OS(═O)₂N(R)—, —N(R)S(═O)₂O—, —SC(═O)—, —C(═O)S—, —OC(═S)N(R)—, —N(R)C(═S)O—, —C(═S)N(R)—, —N(R)C(═S)—, —N(R)C(═S)N(R)—, —C(═S)—, —OC(═S)—, —C(═S)O—, —S(═O)₂N(R)—, —N(R)S(═O)₂—, —S(═O)₂N(R)C(═O)—, or —C(═O)N(R)S(═O)₂—;

Y₂ is —OCH₂—, —SCH₂—, —N(R)CH₂—, —N(R)C(═O)—, —C(═O)N(R)—, —S(═O)₂CH₂—, —S(═O)CH₂—, —CH₂O—, —CH₂CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, ethylene, propylene, n-butylene, —O—C₁₋₄ alkylene-N(R)C(═O)—, —O—C₁₋₄ alkylene-C(═O)N(R)—, —N(R)C(═O)—C₁₋₄ alkylene-O—, —C(═O)N(R)—C₁₋₄ alkylene-O—, —C₁₋₄alkylene-S(═O)₂—, —C₁₋₄alkylene-S(═O)—, —S(═O)₂—C₁₋₄alkylene-, —S(═O)—C₁₋₄ alkylene-, —C₁₋₄alkylene-SO₂N(R)—, —C₁₋₄alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄ alkylene-O—, —S—C₁₋₄ alkylene-, —C₁₋₄ alkylene-S—, —C₁₋₄alkylene-S—C₁₋₄ alkylene-, —N(R)—C₁₋₄ alkylene-, —C₁₋₄alkylene-N(R)—, —C₁₋₄alkylene-N(R)—C₁₋₄ alkylene-, —C₁₋₄alkylene-C(═O)—O—C₁₋₄alkylene-, —C₁₋₄alkylene-O—C(═O)—C₁₋₄ alkylene-, —C₁₋₄ alkylene-C(═O)—N(R)—C₁₋₄ alkylene-, —C₁₋₄alkylene-N(R)—C(═O)—C(═O)—C₁₋₄ alkylene-, —C(═O)—N(R)—C₁₋₄ alkylene-SO₂N(R)—, or —N(R)—C(═O)—C₁₋₄ alkylene-SO₂N(R)—;

Z₀ is carbocycle, cycloalkyl, cycloalkenyl, heterocycle, heterocyclonoyl, aryl, heteroaryl, carbocycloalkyl, heterocyclylalkyl, arylalkyl, arylalkenyl, heteroarylalkyl, heteroarylalkenyl, heteroarylalkynyl, or arylalkynyl, wherein any of the foregoing groups are optionally substituted at least once with alkyl, alkylene, alkenyl, alkenylene, alkynyl, alkynylene, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide;

wherein any alkylene or alkenylene group is optionally independently substituted with C₁₋₄ alkyl, halo, C₁₋₄haloalkyl, or C₃ or C₄ cycloalkyl;

wherein for the purposes of Y and Y₁, R is H, halo, C₁₋₄ alkyl, C₁₋₄ alkenyl, or C₁₋₄ alkynyl;

wherein for the purpose of Y₂, R is H, halo, C₁₋₅ alkyl, C₁₋₅ alkenyl, C₁₋₅ alkynyl, or forms a heterocycle with a carbon atom of Z₀; and

with the proviso that the compound is NOT:
ethyl 3-(pyridin-3-yl)-4-({4-[(3-{[(pyridin-3-ylmethyl)carbamoyl]amino}benzyl)oxy]phenyl}sulfonyl)butanoate;
4-({4-(3-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl) sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid;
4-({4-[(4-fluoro-3-{[(pyridin-3-ylmethyl)carbamoyl]amino}benzyl)oxy]phenyl}sulfonyl)-3-(pyridin-3-yl)butanoic acid;
1,1′-butane-1,4-diylbis[3-(pyridin-3-ylmethyl)urea];
1-[(6-methoxypyridin-3-yl)methyl]-3-[3-(3-methylphenoxy)propyl]urea; or
1-[3-(2-fluorophenoxy)propyl]-3-[(6-methoxypyridin-3-yl)methyl]urea.

In some embodiments the present invention provides compounds of Formula Ia

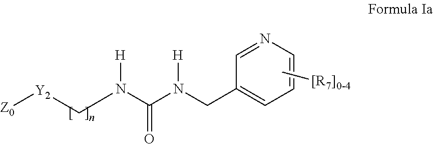

and pharmaceutically acceptable salts and solvates thereof;
wherein:

Z₀ and Y₂ are as defined for Formula I above;

n is 3, 4, 5, 6, or 7;

any methylene group is optionally independently substituted with C₁₋₄alkyl, halo, C₁₋₄ haloalkyl, or C₃ or C₄ cycloalkyl;

R₇, if present one or more times, replaces a hydrogen atom on the pyridinyl ring and is independently selected from halo, C₁₋₅alkyl, nitro, cyano, C₁₋₅alkoxy, C-amido, N-amido, trihalomethyl, C-carboxy, O-carboxy, trihalomethyl, C-carboxy, O-carboxy, sulfonamide, amino, hydroxyl, mercapto, alkylthio, sulfonyl, and sulfinyl; and with the proviso that the compound is NOT:
1,1′-butane-1,4-diylbis[3-(pyridin-3-ylmethyl)urea].

In some embodiments the present invention provides compounds of Formula Ial

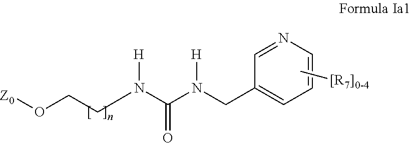

and pharmaceutically acceptable salts and solvates thereof;
wherein:

Z₀ is as defined for Formula I above;

n is 3, 4, 5, 6, or 7;

any methylene group is optionally independently substituted with C₁₋₄alkyl, halo, C₁₋₄ haloalkyl, or C₃ or C₄ cycloalkyl; and

R₇ is as defined for Formula Ia.

In some embodiments the present invention provides compounds of Formula Ia2

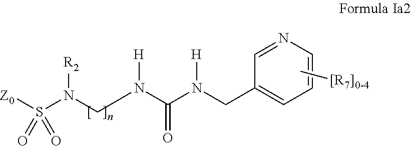

and pharmaceutically acceptable salts and solvates thereof;
wherein:

Z₀ is as defined for Formula I above;

n is 3, 4, 5, 6, or 7;

any methylene group is optionally independently substituted with C₁₋₄ alkyl, halo, C₁₋₄ haloalkyl, or C₃ or C₄ cycloalkyl;

R₂ is H, C₁₋₅₅ alkyl, C₁₋₅₅ alkenyl, or C₁₋₅alkynyl; and

R₇ is as defined for Formula Ia.

In some embodiments the present invention provides compounds of Formula Ib

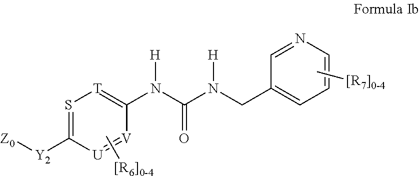

and pharmaceutically acceptable salts and solvates thereof;
wherein:

Z₀ and Y₂ are as defined for Formula I above;

any methylene group is optionally independently substituted with C₁₋₄ alkyl, halo, C₁₋₄ haloalkyl, or C₃ or C₄ cycloalkyl;

R₆ and R₇ are each independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl; and

S, T, U, and V are carbon or nitrogen, provided that when S, T, U, or V is nitrogen, then there is no substituent on the nitrogen.

In some embodiments the present invention provides compounds of Formula Ibl

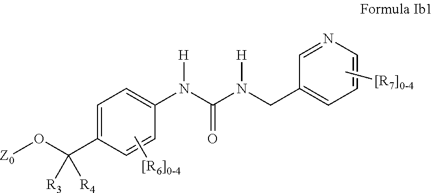

and pharmaceutically acceptable salts and solvates thereof;
wherein:

Z₀ is as defined for Formula I above;

any methylene group is optionally independently substituted with C₁₋₄alkyl, halo, C₁₋₄ haloalkyl, or C₃ or C₄ cycloalkyl;

R₃ and R₄ are each independently H or C₁₋₄alkyl, or R₃ and R₄, taken together with the carbon to which they are attached, form a cyclopropyl or cyclobutyl ring; and

R₆ and R₇ are areas defined for Formula Ib above.

In some embodiments the present invention provides compounds of Formula Ib2

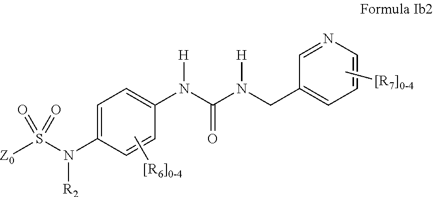

and pharmaceutically acceptable salts and solvates thereof;
wherein:

Z₀ is as defined for Formula I above;

any methylene group is optionally independently substituted with C₁₋₄alkyl, halo, C₁₋₄ haloalkyl, or C₃ or C₄ cycloalkyl;

R₂ is H, C₁₋₅alkyl, C₁₋₅alkenyl, or C₁₋₅alkynyl; and

R₆ and R₇ are as defined for Formula Ib above.

In some embodiments the present invention provides compounds of Formula Ib3

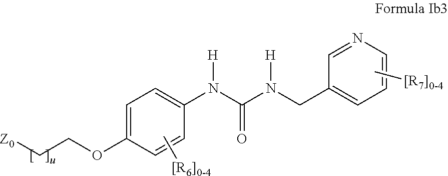

and pharmaceutically acceptable salts and solvates thereof;
wherein:

Z₀ is as defined for Formula I above;

u is 0 or 1;

any methylene group is optionally independently substituted with C₁₋₄ alkyl, halo, C₁₋₄ haloalkyl, or C₃ or C₄ cycloalkyl; and

R₆ and R₇ are as defined for Formula Ib above.

In some embodiments the present invention provides compounds of Formula Ic

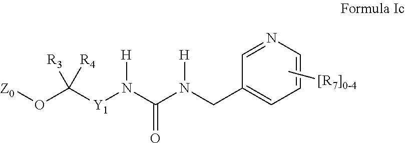

and pharmaceutically acceptable salts and solvates thereof;
wherein:

Z₀ and Y₁ are as defined for Formula I above;

any methylene group is optionally independently substituted with C₁₋₄ alkyl, halo, C₁₋₄ haloalkyl, or C₃ or C₄ cycloalkyl;

R₃ and R₄ are each independently H or C₁₋₄alkyl, or R₃ and R₄, taken together with the carbon to which they are attached, form a cyclopropyl or cyclobutyl ring;

R₇, if present one or more times, replaces a hydrogen atom on the pyridinyl ring and is independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl; and

with the proviso that the compound is NOT:
ethyl 3-(pyridin-3-yl)-4-({4-[(3-{[(pyridin-3-ylmethyl)carbamoyl]amino}benzyl)oxy]phenyl}sulfonyl)butanoate;
4-({4-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl) sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid; or
4-({4-[(4-fluoro-3-{[(pyridin-3-ylmethyl)carbamoyl]amino}benzyl)oxy]phenyl}sulfonyl)-3-(pyridin-3-yl)butanoic acid.

In some embodiments the present invention provides compounds of Formula Id

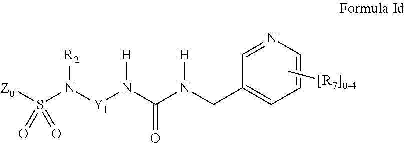

and pharmaceutically acceptable salts and solvates thereof;
wherein:

Z₀ and Y₁ are as defined for Formula I above;

any methylene group is optionally independently substituted with C₁₋₄ alkyl, halo, C₁₋₄ haloalkyl, or C₃ or C₄ cycloalkyl;

R₂ is H, C₁₋₅alkyl, C₁₋₅alkenyl, or C₁₋₅alkynyl; and

R₇, if present one or more times, replaces a hydrogen atom on the pyridinyl ring and is independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl.

The present invention further provides compounds of Formula II

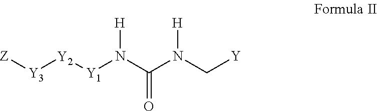

and pharmaceutically acceptable salts and solvates thereof;
wherein:

Z is hydro, halo, C₁₋₅alkyl, nitro, cyano, C₁₋₅alkoxy, C-amido, N-amido, trihalomethyl, C-carboxy, O-carboxy, trihalomethyl, C-carboxy, O-carboxy, sulfonamide, amino, hydroxyl, mercapto, alkylthio, sulfonyl, or sulfinyl, wherein C₁₋₅alkyl, C₁₋₅ alkoxy, C-amido, N-amido, amino, and alkylthio are each optionally substituted with heteroaryl, heterocyclo, cycloalkyl, alkoxy, or amino;

or Z is Z₀, as defined for Formula I above;

Y and Y₁ R is as defined for Formula I above, wherein for the purpose of Y₂, R is H, C₁₋₅ alkyl, C₁₋₅ alkenyl, C₁₋₅ alkynyl, or forms a heterocycle with a carbon atom of Y₃;

Y₃ is aryl or heteroaryl, wherein any ring carbon is optionally independently substituted with halo, C₁₋₅alkyl, nitro, cyano, trihalomethyl, C₁₋₅alkoxy, C-amido, N-amido, sulfonamide, amino, aminosulfonyl, hydroxyl, mercapto, alkylthio, sulfonyl, or sulfinyl, wherein C₁₋₅alkyl, C₁₋₅alkoxy, C-amido, N-amido, amino, and alkylthio are each optionally substituted with heteroaryl, heterocyclo, cycloalkyl, alkoxy, or amino;

any alkylene or alkenylene group is optionally independently substituted with C₁₋₄ alkyl, halo, C₁₋₄haloalkyl, or C₃ or C₄ cycloalkyl; and

with the proviso that the compound is NOT:
1-[(6-methoxypyridin-3-yl)methyl]-3-[3-(3-methylphenoxy)propyl]urea;
1-[3-(2-fluorophenoxy)propyl]-3-[(6-methoxypyridin-3-yl)methyl]urea;
ethyl 3-(pyridin-3-yl)-4-({4-[(3-{[(pyridin-3-ylmethyl)carbamoyl]amino}benzyl)oxy]phenyl}sulfonyl)butanoate;
4-({4-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl) sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid; or
4-({4-[(4-fluoro-3-{[(pyridin-3-ylmethyl)carbamoyl]amino}benzyl)oxy]phenyl}sulfonyl)-3-(pyridin-3-yl)butanoic acid.

In some embodiments the present invention provides compounds of Formula IIa

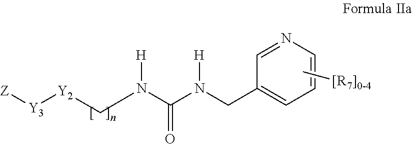

and pharmaceutically acceptable salts and solvates thereof;
wherein

Z, Y₂, and Y₃ are as defined for Formula II above;

n is 3, 4, 5, 6, or 7;

any methylene group is optionally independently substituted with C₁₋₄alkyl, halo, C₁₋₄ haloalkyl, or C₃ or C₄ cycloalkyl; and

R₇, if present one or more times, replaces a hydrogen atom on the pyridinyl ring and is independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl.

In some embodiments the present invention provides compounds of Formula IIa1

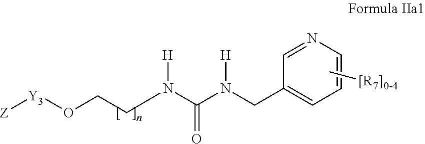

and pharmaceutically acceptable salts and solvates thereof;
wherein:

Z and Y₃ are as defined for Formula II above;

n is 3, 4, 5, 6, or 7;

any methylene group is optionally independently substituted with C₁₋₄alkyl, halo, C₁₋₄ haloalkyl, or C₃ or C₄ cycloalkyl; and

R₇ is as defined for Formula IIa above.

In some embodiments the present invention provides compounds of Formula IIa2

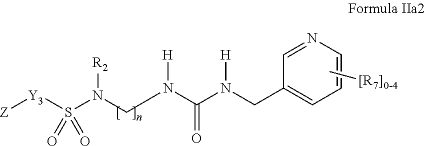

and pharmaceutically acceptable salts and solvates thereof;
wherein:

Z and Y₃ are as defined for Formula II above;

n is 3, 4, 5, 6, or 7;

any methylene group is optionally independently substituted with C₁₋₄ alkyl, halo, C₁₋₄ haloalkyl, or C₃ or C₄ cycloalkyl;

R₂ is H, C₁₋₅alkyl, C₁₋₅alkenyl, or C₁₋₅alkynyl; and

R₇ is as defined for Formula IIa above.

In some embodiments the present invention provides compounds of Formula IIa3

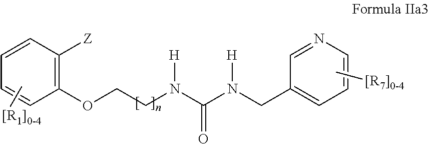

and pharmaceutically acceptable salts and solvates thereof;
wherein:

Z is as defined for Formula II above;

n is 3, 4, 5, 6, or 7;

any methylene group is optionally independently substituted with C₁₋₄ alkyl, halo, C₁₋₄ haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₇ is as defined for Formula IIa above.

In some embodiments the present invention provides compounds of Formula IIa4

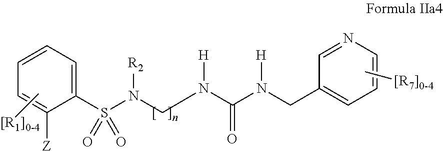

and pharmaceutically acceptable salts and solvates thereof;
wherein:

Z is as defined for Formula II above;

n is 3, 4, 5, 6, or 7;

any methylene group is optionally independently substituted with C₁₋₄ alkyl, halo, C₁₋₄ haloalkyl, or C₃ or C₄ cycloalkyl;

R₁, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino;

R₂ is H, C₁₋₅alkyl, C₁₋₅alkenyl, or C₁₋₅alkynyl; and

R₇ is as defined for Formula IIa above.

In some embodiments the present invention provides compounds of Formula IIb

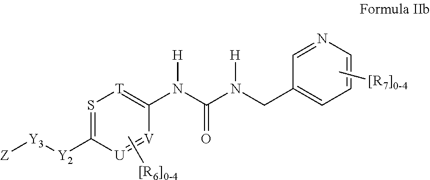

and pharmaceutically acceptable salts and solvates thereof;
wherein:

Z, Y₂, and Y₃ are as defined for Formula II above,

any methylene group is optionally independently substituted with C₁₋₄alkyl, halo, C₁₋₄ haloalkyl, or C₃ or C₄ cycloalkyl;

R₆ and R₇ are each independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl; and

S, T, U, and V are carbon or nitrogen, provided that when S, T, U, or V is nitrogen, then there is no substituent on the nitrogen.

In some embodiments the present invention provides compounds of Formula IIb1

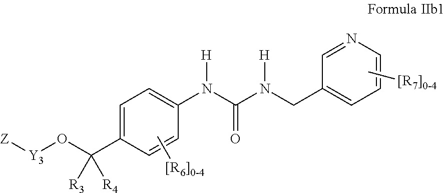

and pharmaceutically acceptable salts and solvates thereof;
wherein:

Z and Y₃ are as defined for Formula II above,

any methylene group is optionally independently substituted with C₁₋₄ alkyl, halo, C₁₋₄ haloalkyl, or C₃ or C₄ cycloalkyl;

R₃ and R₄ are each independently H or C₁₋₄alkyl, or R₃ and R₄, taken together with the carbon to which they are attached, form a cyclopropyl or cyclobutyl ring; and

R₆ and R₇ are as defined for Formula IIb above.

In some embodiments the present invention provides compounds of Formula IIb2

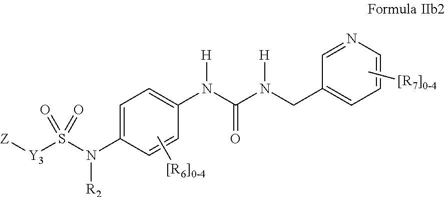

and pharmaceutically acceptable salts and solvates thereof;
wherein:

Z and Y₃ are as defined for Formula II above;

any methylene group is optionally independently substituted with C₁₋₄alkyl, halo, C₁₋₄ haloalkyl, or C₃ or C₄ cycloalkyl;

R₂ is H, C₁₋₅alkyl, C₁₋₅alkenyl, or C₁₋₅alkynyl; and

R₆ and R₇ are as defined for Formula IIb above.

In some embodiments the present invention provides compounds of Formula IIb3

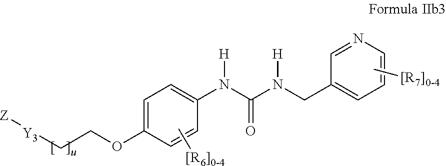

and pharmaceutically acceptable salts and solvates thereof;
wherein:

Z and Y₃ are as defined for Formula II above,

u is 0 or 1;

any methylene group is optionally independently substituted with C₁₋₄ alkyl, halo, C₁₋₄ haloalkyl, or C₃ or C₄ cycloalkyl; and

R₆ and R₇ are as defined for Formula IIb above.

In some embodiments the present invention provides compounds of Formula IIb4

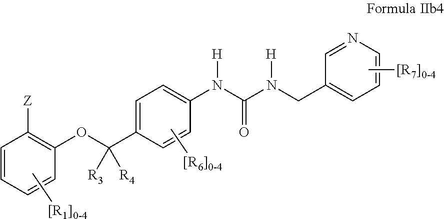

and pharmaceutically acceptable salts and solvates thereof;
wherein:

Z is as defined for Formula II above;

any methylene group is optionally independently substituted with C₁₋₄alkyl, halo, C₁₋₄ haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino;

R₃ and R₄ are each independently H or C₁₋₄alkyl, or R₃ and R₄, taken together with the carbon to which they are attached, form a cyclopropyl or cyclobutyl ring; and

R₆ and R₇ are as defined for Formula IIb above.

In some embodiments the present invention provides compounds of Formula IIb5

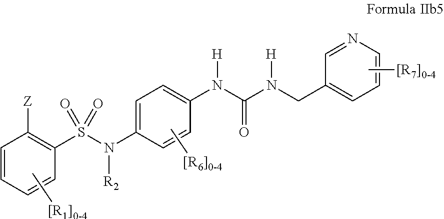

and pharmaceutically acceptable salts and solvates thereof;
wherein:

Z is as defined for Formula II above;

any methylene group is optionally independently substituted with C₁₋₄alkyl, halo, C₁₋₄ haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino;

R₂ is H, C₁₋₅alkyl, C₁₋₅alkenyl, or C₁₋₅alkynyl; and

R₆ and R₇ are as defined for Formula IIb above.

In some embodiments the present invention provides compounds of Formula IIb6

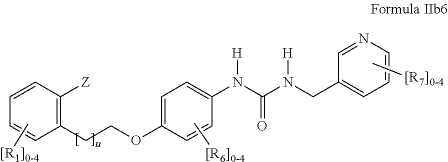

and pharmaceutically acceptable salts and solvates thereof;
wherein:

Z is as defined for Formula II above;

u is 0 or 1;

any methylene group is optionally independently substituted with C₁₋₄ alkyl, halo, C₁₋₄ haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₆ and R₇ are as defined for Formula IIb above.

In some embodiments the present invention provides compounds of Formula IIb7

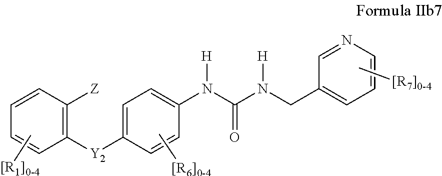

and pharmaceutically acceptable salts and solvates thereof;
wherein:

Z and Y₂ are as defined for Formula II above;

any methylene group is optionally independently substituted with C₁₋₄alkyl, halo, C₁₋₄ haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₆ and R₇ are as defined for Formula IIb above.

In some embodiments the present invention provides compounds of Formula IIc

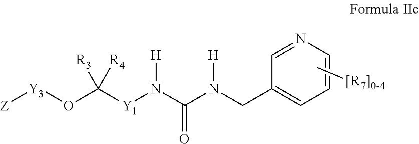

and pharmaceutically acceptable salts and solvates thereof;
wherein:

Z, Y₁, and Y₃ are as defined for Formula II above;

any alkylene or alkenylene group is optionally independently substituted with C₁₋₄ alkyl, halo, C₁₋₄haloalkyl, or C₃ or C₄ cycloalkyl;

R₃ and R₄ are each independently H or C₁₋₄alkyl, or R₃ and R₄, taken together with the carbon to which they are attached, form a cyclopropyl or cyclobutyl ring; and

R₇, if present one or more times, replaces a hydrogen atom on the pyridinyl ring and is independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl.

In some embodiments the present invention provides compounds of Formula IIc1

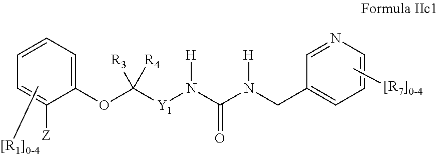

and pharmaceutically acceptable salts and solvates thereof;
wherein:

Z and Y₁ are as defined in Formula II above;

any alkylene or alkenylene group is optionally independently substituted with C₁₋₄ alkyl, halo, C₁₋₄haloalkyl, or C₃ or C₄ cycloalkyl;

R₁, if present one or more times, is independently selected from halo, C₁₋₅ alkyl, nitro, cyano, alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₃, R₄, and R₇ are as defined for Formula IIc.

In some embodiments the present invention provides compounds of Formula lid

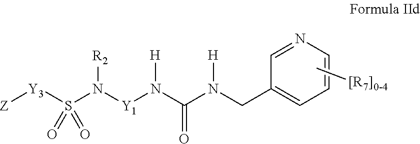

and pharmaceutically acceptable salts and solvates thereof;
wherein:

Z, Y₁, and Y₃ are as defined for Formula II above;

any alkylene or alkenylene group is optionally independently substituted with C₁₋₄alkyl, halo, C₁₋₄haloalkyl, or C₃ or C₄ cycloalkyl;

R₂ is H, C₁₋₅alkyl, C₁₋₅alkenyl, or C₁₋₅alkynyl; and

R₇, if present one or more times, replaces a hydrogen atom on the pyridinyl ring and is independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl.

In some embodiments the present invention provides compounds of Formula IId1

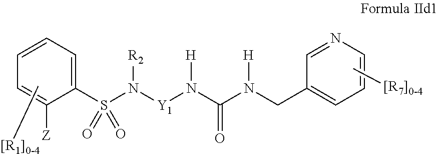

and pharmaceutically acceptable salts and solvates thereof;
wherein:

Z and Y₁ are as defined for Formula II above;

any alkylene or alkenylene group is optionally independently substituted with C₁₋₄ alkyl, halo, C₁₋₄haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₂ and R₇ are as defined for Formula IId.

The present invention further provides compounds of Formula III

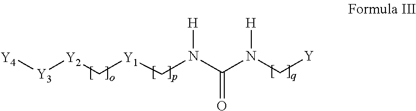

and pharmaceutically acceptable salts and solvates thereof;
wherein:

Y, Y₁, Y₂, and Y₃ are as defined for Formula II;

Y₄ is optionally present, and when present is aryl, heteroaryl, carbocycle, or heterocycle, wherein any ring atom is optionally independently substituted with halo, C₁₋₅ alkyl, nitro, cyano, trihalomethyl, C₁₋₅alkoxy, C-amido, N-amido, sulfonamide, amino, aminosulfonyl, hydroxyl, mercapto, alkylthio, sulfonyl, sulfinyl, wherein C₁₋₅ alkyl, C₁₋₅ alkoxy, C-amido, N-amido, amino, and alkylthio are each optionally substituted with heteroaryl, heterocyclo, cycloalkyl, alkoxy, or amino;

o, p, and q are each independently 0, 1, or 2;

any alkylene or alkenylene group of the o, p, and q regions and of Y₂ is optionally substituted with unsubstituted C₁₋₄ alkyl, halo, unsubstituted C₁₋₄ haloalkyl, or unsubstituted C₃ or C₄ cycloalkyl;

with the proviso that when p is 0, Y₁ is divalent phenyl, Y₂ is —C(═O)N(H)— or —OC(H)₂C(═O)N(H)—, and Y₃ is phenyl or pyridinyl, then either Y₄ is present or any substituent on Y₃ is not —C(═O)NH₂; and

with the proviso that the compound is NOT:
1-(6-methoxy-3-pyridyl)-3-[[4-(3-pyridylmethoxy)phenyl]methyl]urea;
1-[(6-methoxypyridin-3-yl)methyl]-3-[3-(3-methylphenoxy)propyl]urea;
1-[3-(2-fluorophenoxy)propyl]-3-[(6-methoxypyridin-3-yl)methyl]urea;
ethyl 3-(pyridin-3-yl)-4-({4-[(3-{[(pyridin-3-ylmethyl)carbamoyl]amino}benzyl)oxy]phenyl}sulfonyl)butanoate;
4-({4-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl) sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid;
4-({4-[(4-fluoro-3-{[(pyridin-3-ylmethyl)carbamoyl]amino}benzyl)oxy]phenyl}sulfonyl)-3-(pyridin-3-yl)butanoic acid;
Benzoic acid, 2-hydroxy-4-[[(3-pyridinylamino)carbonyl]amino]-, phenyl ester;
Benzamide, N-(3-amino-4-pyridinyl)-4-[[[[(3-pyridinylmethyl)amino]carbonyl]amino]methyl]-;
Benzamide, N-(2-amino-3-pyridinyl)-4-[[[[(3-pyridinylmethyl)amino]carbonyl]amino]methyl]-;
Benzamide, N-(2-amino-5-fluorophenyl)-4-[[[[(3-pyridinylmethyl)amino]carbonyl]amino]methyl]-;
Benzamide, N-(2-hydroxyphenyl)-4-[[[[(3-pyridinylmethyl)amino]carbonyl]amino]methyl]-;
Benzamide, N-(2-amino-5-chlorophenyl)-4-[[[[(3-pyridinylmethyl)amino]carbonyl]amino]methyl]-;
Benzamide, 2-chloro-5-nitro-N-[4-[[(4-pyridinylamino)carbonyl]amino]phenyl]-;
Benzamide, N-[4-[[[3-(diethylamino)propyl]amino]carbonyl]phenyl]-4-[[(3-pyridinylamino) carbonyl]amino]-;
Benzamide, N-(2-aminophenyl)-4-[[[(3-pyridinylamino)carbonyl]amino]methyl]-;
Benzamide, N-(2-aminophenyl)-4-[2-[[[(3-pyridinylmethyl)amino]carbonyl]amino]ethyl]-;
Benzamide, N-(2-aminophenyl)-4-[[[[(3-pyridinylmethyl)amino]carbonyl]amino]methyl]-;
Benzoic acid, 2-hydroxy-4-[[(3-pyridinylamino)carbonyl]amino]-, phenyl ester;
1,3-Benzenedicarboxamide, N,N′-bis[3-(diethylamino)propyl]-5-[[4-[[(4-pyridinylamino)carbonyl]amino]benzoyl]amino]-;
Urea, N-[4-(phenylmethoxy)phenyl]-N′-[2-(3-pyridinyl)ethyl]-;
Urea, N-[4-(phenylmethoxy)phenyl]-N′-3-pyridinyl-;
Urea, N-(6-methyl-3-pyridinyl)-N′-[2-[2-(phenylmethoxy)phenyl]ethyl]-;
Urea, N-(6-methoxy-3-pyridinyl)-N′-[4-(phenylmethoxy)phenyl]-;
4,6-Pyrimidinedicarboxamide, N4-[[4-[[[(2,6-dichloro-4-pyridinyl)amino]carbonyl]amino]phenyl]methyl]-N-6-[(3-methoxyphenyl)methyl]-;
Benzenesulfonamide, 4-fluoro-N-[4-[[(3-pyridinylamino)carbonyl]amino]phenyl]-; or
Hexanamide, 2-[2,4-bis(1,1-dimethylpropyl)phenoxy]-N-[2-chloro-4-[[[(2-chloro-3-pyridinyl)amino]carbonyl]amino]-5-hydroxyphenyl]-.

In some embodiments the present invention provides compounds of Formula IIIa

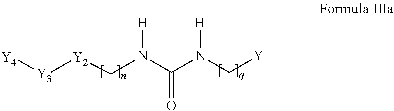

and pharmaceutically acceptable salts and solvates thereof;
wherein:

Y is 3-pyridinyl or 4-pyridinyl, optionally substituted as defined for Y for Formula I;

Y₂, Y₃, Y₄, and q are as defined for Formula III above;

n is 3, 4, 5, 6, or 7; and

any methylene group of Y₂ and the n and q regions is optionally independently substituted with C₁₋₄alkyl, halo, C₁₋₄ haloalkyl, or C₃ or C₄ cycloalkyl.

In some embodiments the present invention provides compounds of Formula IIIa1

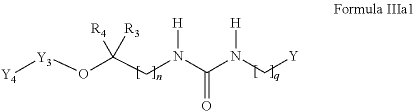

and pharmaceutically acceptable salts and solvates thereof;
wherein:

Y is 3-pyridinyl or 4-pyridinyl, optionally substituted as defined for Y for Formula I;

Y₃, Y₄, and q are as defined for Formula III above;

n is 3, 4, 5, 6, or 7; any methylene group of the n and q regions is optionally independently substituted with C₁₋₄ alkyl, halo, C₁₋₄haloalkyl, or C₃ or C₄ cycloalkyl; and

R₃ and R₄ are each independently H, halo, or C₁₋₄alkyl, or R₃ and R₄ taken together form a cyclopropyl or cyclobutyl ring.

In some embodiments the present invention provides compounds of Formula IIIa2

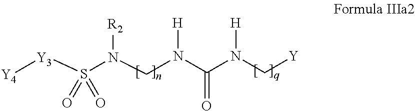

and pharmaceutically acceptable salts and solvates thereof;
wherein:

Y is 3-pyridinyl or 4-pyridinyl, optionally substituted as defined for Y for Formula I;

Y₃, Y₄, and q are as defined for Formula III above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl; and

R₂ is H, halo, C₁₋₅alkyl, C₁₋₅alkenyl, or C₁₋₅alkynyl.

In some embodiments the present invention provides compounds of Formula IIIa3

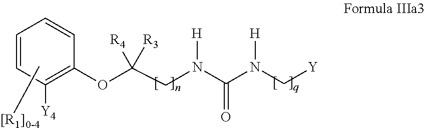

and pharmaceutically acceptable salts and solvates thereof;
wherein:

Y is 3-pyridinyl or 4-pyridinyl, optionally substituted as defined for Y for Formula I;

Y₄ and q are as defined for Formula III above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅alkoxy, C-amido, N-amido, amino, aminoalkyl, and alkylthio are each optionally substituted with heteroaryl, heterocyclo, cycloalkyl, alkoxy, or amino; and

R₃ and R₄ are each independently H, halo, or C₁₋₄alkyl, or R₃ and R₄, taken together with the carbon to which they are attached, form a cyclopropyl or cyclobutyl ring.

In some embodiments the present invention provides compounds of Formula IIIa4

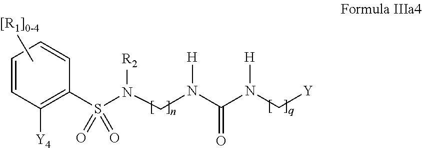

and pharmaceutically acceptable salts and solvates thereof;
wherein:

Y is 3-pyridinyl or 4-pyridinyl, optionally substituted as defined for Y for Formula I;

Y₄ and q are as defined for Formula III above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino; and

R₂ is H, halo, C₁₋₅alkyl, C₁₋₅alkenyl, or C₁₋₅alkynyl.

In some embodiments the present invention provides compounds of Formula IIIa5

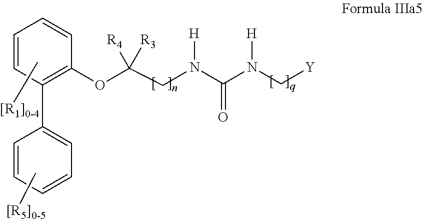

and pharmaceutically acceptable salts and solvates thereof;
wherein:

Y is 3-pyridinyl or 4-pyridinyl, optionally substituted as defined for Y for Formula I;

q is as defined for Formula III above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino; and

R₃ and R₄ are each independently H, halo, or C₁₋₄alkyl, or R₃ and R₄, taken together with the carbon to which they are attached, form a cyclopropyl or cyclobutyl ring.

In some embodiments the present invention provides compounds of Formula IIIa6

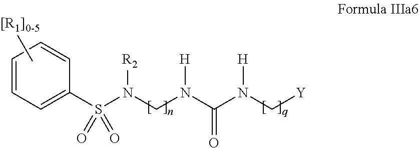

and pharmaceutically acceptable salts and solvates thereof;
wherein:

Y is 3-pyridinyl or 4-pyridinyl, optionally substituted as defined for Y for Formula I;

q is as defined for Formula III above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino; and

R₂ is H, halo, C₁₋₅alkyl, C₁₋₅alkenyl, or C₁₋₅alkynyl.

In some embodiments the present invention provides compounds of Formula IIIb

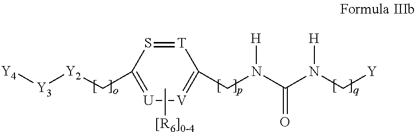

and pharmaceutically acceptable salts and solvates thereof;
wherein:

Y is 3-pyridinyl or 4-pyridinyl, optionally substituted as defined for Y for Formula I;

o, p, q, Y₂, Y₃, and Y₄ are as defined for Formula III above;

any methylene group of the o, p, and q regions and Y₂ is optionally independently substituted with C₁₋₄alkyl, halo, C₁₋₄ haloalkyl, or C₃ or C₄ cycloalkyl;

R₆, if present one or more times, is independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl;

wherein S, T, U, and V are carbon or nitrogen, provided that when S, T, U, or V is nitrogen, then there is no substituent on the nitrogen;

with the proviso that when p is 0, Y₂ is —C(═O)N(H)— or —OC(H)₂C(═O)N(H)—, and Y₃ is phenyl or pyridinyl, then either Y₄ is present or any substituent on Y₃ is not —C(═O)NH₂; and with the proviso that the compound is NOT
1-(6-methoxy-3-pyridyl)-3-[[4-(3-pyridylmethoxy)phenyl]methyl]urea,
ethyl 3-(pyridin-3-yl)-4-({4-[(3-{[(pyridin-3-ylmethyl)carbamoyl]amino}benzyl)oxy]phenyl}sulfonyl)butanoate;
4-({4-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl) sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid;
4-({4-[(4-fluoro-3-{[(pyridin-3-ylmethyl)carbamoyl]amino}benzyl)oxy]phenyl}sulfonyl)-3-(pyridin-3-yl)butanoic acid;
Benzoic acid, 2-hydroxy-4-[[(3-pyridinylamino)carbonyl]amino]-, phenyl ester,
Benzamide, N-(3-amino-4-pyridinyl)-4-[[[[(3-pyridinylmethyl)amino]carbonyl]amino]methyl]-,
Benzamide, N-(2-amino-3-pyridinyl)-4-[[[[(3-pyridinylmethyl)amino]carbonyl]amino]methyl]-,
Benzamide, N-(2-amino-5-fluorophenyl)-4-[[[[(3-pyridinylmethyl)amino]carbonyl]amino]methyl]-,
Benzamide, N-(2-hydroxyphenyl)-4-[[[[(3-pyridinylmethyl)amino]carbonyl]amino]methyl]-,
Benzamide, N-(2-amino-5-chlorophenyl)-4-[[[[(3-pyridinylmethyl)amino]carbonyl]amino]methyl]-,
Benzamide, 2-chloro-5-nitro-N-[4-[[(4-pyridinylamino)carbonyl]amino]phenyl]-,
Benzamide, N-[4-[[[3-(diethylamino)propyl]amino]carbonyl]phenyl]-4-[[(3-pyridinylamino) carbonyl]amino]-,
Benzamide, N-(2-aminophenyl)-4-[[[(3-pyridinylamino)carbonyl]amino]methyl]-,
Benzamide, N-(2-aminophenyl)-4-[2-[[[(3-pyridinylmethyl)amino]carbonyl]amino]ethyl]-,
Benzamide, N-(2-aminophenyl)-4-[[[[(3-pyridinylmethyl)amino]carbonyl]amino]methyl]-,
Benzoic acid, 2-hydroxy-4-[[(3-pyridinylamino)carbonyl]amino]-, phenyl ester,
1,3-Benzenedicarboxamide, N,N′-bis[3-(diethylamino)propyl]-5-[[4-[[(4-pyridinylamino)carbonyl]amino]benzoyl]amino]-,
Urea, N-[4-(phenylmethoxy)phenyl]-N′-[2-(3-pyridinyl)ethyl]-,
Urea, N-[4-(phenylmethoxy)phenyl]-N′-3-pyridinyl-,
Urea, N-(6-methyl-3-pyridinyl)-N′-[2-[2-(phenylmethoxy)phenyl]ethyl]-,
Urea, N-(6-methoxy-3-pyridinyl)-N′-[4-(phenylmethoxy)phenyl]-,
4,6-Pyrimidinedicarboxamide, N4-[[4-[[[(2,6-dichloro-4-pyridinyl)amino]carbonyl]amino]phenyl]methyl]-N-6-[(3-methoxyphenyl)methyl]-,
Benzenesulfonamide, 4-fluoro-N-[4-[[(3-pyridinylamino)carbonyl]amino]phenyl]-, or
Hexanamide, 2-[2,4-bis(1,1-dimethylpropyl)phenoxy]-N-[2-chloro-4-[[[(2-chloro-3-pyridinyl)amino]carbonyl]amino]-5-hydroxyphenyl]-.

In some embodiments the present invention provides compounds of Formula IIIb1

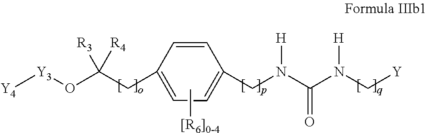

and pharmaceutically acceptable salts and solvates thereof;
wherein:

Y is 3-pyridinyl or 4-pyridinyl, optionally substituted as defined for Y for Formula I;

o, p, q, Y₃, and Y₄ are as defined for Formula III above;

any methylene group of the o, p, and q regions is optionally independently substituted with C₁₋₄alkyl, halo, C₁₋₄haloalkyl, or C₃ or C₄ cycloalkyl;

R₃ and R₄ are each independently H, halo, or C₁₋₄alkyl, or R₃ and R₄, taken together with the carbon to which they are attached, form a cyclopropyl or cyclobutyl ring; and

R₆ is as defined for Formula IIIb above.

In some embodiments the present invention provides compounds of Formula IIIb2

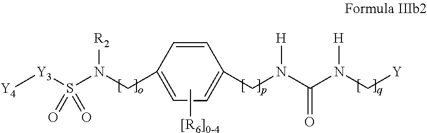

and pharmaceutically acceptable salts and solvates thereof;
wherein:

Y is 3-pyridinyl or 4-pyridinyl, optionally substituted as defined for Y for Formula I;

o, p, q, Y₃, and Y₄ are as defined for Formula III above;

any methylene group of the o, p, and q regions is optionally independently substituted with C₁₋₄alkyl, halo, C₁₋₄haloalkyl, or C₃ or C₄ cycloalkyl;

R₆ is as defined for Formula IIIb above; and

R₂ is H, halo, C₁₋₅alkyl, C₁₋₅alkenyl, or C₁₋₅alkynyl.

In some embodiments the present invention provides compounds of Formula IIIb3

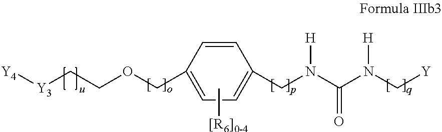

and pharmaceutically acceptable salts and solvates thereof;
wherein:

Y is 3-pyridinyl or 4-pyridinyl, optionally substituted as defined for Y for Formula I;

o, p, q, Y₃, and Y₄ are as defined for Formula III above;

u is 0 or 1;

any methylene group of the o, p, q, and u regions is optionally independently substituted with C₁₋₄alkyl, halo, C₁₋₄haloalkyl, or C₃ or C₄ cycloalkyl; and

R₆ is as defined for Formula IIIb above.

In some embodiments the present invention provides compounds of Formula IIIb4

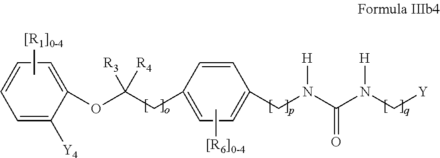

and pharmaceutically acceptable salts and solvates thereof;
wherein:

Y is 3-pyridinyl or 4-pyridinyl, optionally substituted as defined for Y for Formula I;

o, p, q, and Y₄ are as defined for Formula III above;

R₁, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₃ and R₄ are each independently H, halo, or C₁₋₄alkyl, or R₃ and R₄, taken together with the carbon to which they are attached, form a cyclopropyl or cyclobutyl ring;

R₆ is as defined for Formula IIIb above; and

any methylene group of the o, p, and q regions is optionally independently substituted with C₁₋₄alkyl, halo, C₁₋₄haloalkyl, or C₃ or C₄ cycloalkyl.

In some embodiments the present invention provides compounds of Formula IIIb5

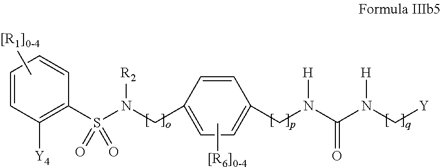

and pharmaceutically acceptable salts and solvates thereof;
wherein:

Y is 3-pyridinyl or 4-pyridinyl, optionally substituted as defined for Y for Formula I;

o, p, q, and Y₄ are as defined for Formula III above;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₂ is H, halo, C₁₋₅alkyl, C₁₋₅alkenyl, or C₁₋₅alkynyl;

R₆ is as defined for Formula IIIb above; and

any methylene group of the o, p, and q regions is optionally independently substituted with C₁₋₄alkyl, halo, C₁₋₄haloalkyl, or C₃ or C₄ cycloalkyl.

In some embodiments the present invention provides compounds of Formula IIIb6

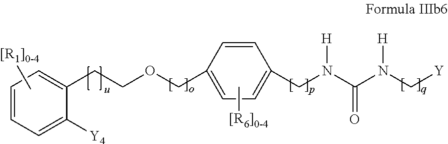

and pharmaceutically acceptable salts and solvates thereof;
wherein:

Y is 3-pyridinyl or 4-pyridinyl, optionally substituted as defined for Y for Formula I;

o, p, q, and Y₄ are as defined for Formula III above;

u is 0 or 1;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₆ is as defined for Formula IIIb above; and

any methylene group of the o, p, q, and u regions is optionally independently substituted with C₁₋₄alkyl, halo, C₁₋₄haloalkyl, or C₃ or C₄ cycloalkyl.

In some embodiments the present invention provides compounds of Formula IIIb7

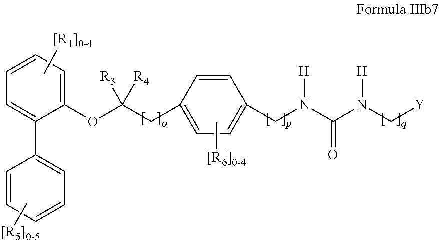

and pharmaceutically acceptable salts and solvates thereof;
wherein:

Y is 3-pyridinyl or 4-pyridinyl, optionally substituted as defined for Y for Formula I;

o, p, and q are as defined for Formula III above;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₃ and R₄ are each independently H, halo, or C₁₋₄alkyl, or R₃ and R₄, taken together with the carbon to which they are attached, form a cyclopropyl or cyclobutyl ring;

R₆ is as defined for Formula IIIb above; and

any methylene group of the o, p, and q regions is optionally independently substituted with C₁₋₄alkyl, halo, C₁₋₄haloalkyl, or C₃ or C₄ cycloalkyl.

In some embodiments the present invention provides compounds of Formula IIIb8

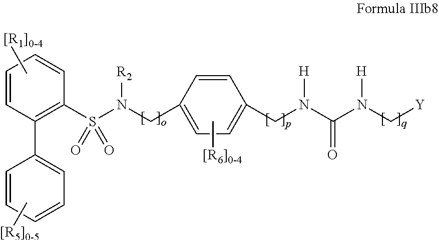

and pharmaceutically acceptable salts and solvates thereof;
wherein:

Y is 3-pyridinyl or 4-pyridinyl, optionally substituted as defined for Y for Formula I;

o, p, and q are as defined for Formula III above;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₂ is H, halo, C₁₋₅alkyl, C₁₋₅alkenyl, or C₁₋₅alkynyl;

R₆ is as defined for Formula IIIb above; and

any methylene group of the o, p, and q regions is optionally independently substituted with C₁₋₄alkyl, halo, C₁₋₄haloalkyl, or C₃ or C₄ cycloalkyl.

In some embodiments the present invention provides compounds of Formula IIIb9

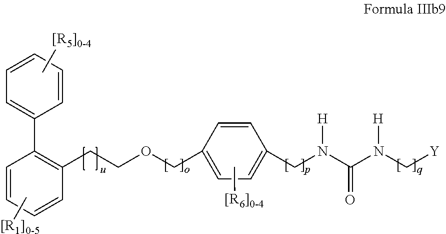

and pharmaceutically acceptable salts and solvates thereof;
wherein:

Y is 3-pyridinyl or 4-pyridinyl, optionally substituted as defined for Y for Formula I;

o, p, and q are as defined for Formula III;

u is 0 or 1;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₆ is as defined if Formula IIIb above; and

any methylene group of the o, p, q, and u regions is optionally independently substituted with C₁₋₄alkyl, halo, C₁₋₄haloalkyl, or C₃ or C₄ cycloalkyl.

In some embodiments the present invention provides compounds of Formula IIIb10

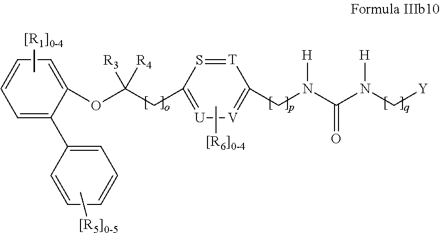

and pharmaceutically acceptable salts and solvates thereof;
wherein:

Y is 3-pyridinyl or 4-pyridinyl, optionally substituted as defined for Y for Formula I;

o, p, and q are as defined for Formula III above;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₃ and R₄ are each independently H, halo, or C₁₋₄ alkyl, or R₃ and R₄, taken together with the carbon to which they are attached, form a cyclopropyl or cyclobutyl ring;

R₆ is as defined for Formula IIIb above;

any methylene group of the o, p, and q regions is optionally independently substituted with C₁₋₄alkyl, halo, C₁₋₄haloalkyl, or C₃ or C₄ cycloalkyl; and

S, T, U, and V are carbon or nitrogen, provided that at least one of S, T, U, and V is nitrogen and that when S, T, U, or V is nitrogen, then there is no substituent on the nitrogen.

In some embodiments the present invention provides compounds of Formula IIIb11

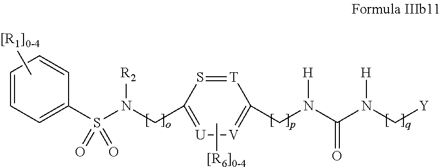

and pharmaceutically acceptable salts and solvates thereof;
wherein:

Y is 3-pyridinyl or 4-pyridinyl, optionally substituted as defined for Y for Formula I;

o, p, and q are as defined for Formula III above;

R₁, if one or both are present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₂ is H, halo, C₁₋₅alkyl, C₁₋₅alkenyl, or C₁₋₅alkynyl;

R₆ is as defined for Formula IIIb above;

any methylene group of the o, p, and q regions is optionally independently substituted with C₁₋₄alkyl, halo, C₁₋₄haloalkyl, or C₃ or C₄ cycloalkyl; and

S, T, U, and V are carbon or nitrogen, provided that at least one of S, T, U, and V is nitrogen and that when S, T, U, or V is nitrogen, then there is no substituent on the nitrogen.

In some embodiments the present invention provides compounds of Formula IIIc

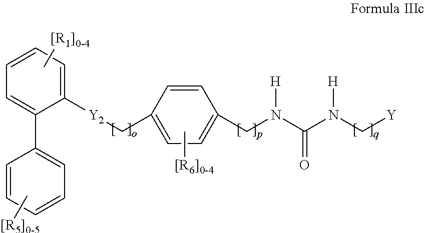

and pharmaceutically acceptable salts and solvates thereof;
wherein:

Y is 3-pyridinyl or 4-pyridinyl, optionally substituted as defined for Y for Formula I;

Y₂, o, p, and q are as defined for Formula III;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;

R₆, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl; and any methylene group of the o, p, and q regions, or Y₂, is optionally independently substituted with C₁₋₄alkyl, halo, C₁₋₄ haloalkyl, or C₃ or C₄ cycloalkyl.

The present invention further provides compounds of Formula IV

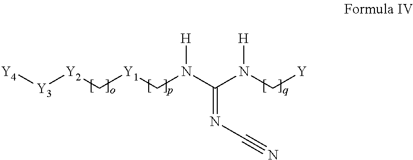

and pharmaceutically acceptable salts and solvates thereof;
wherein:

o, p, q, Y, Y₁, Y₂, Y₃, and Y₄ are as defined for Formula III above;

with the proviso that when Y₁ is divalent phenyl, q is 0, and p is 1, then Y₄ is present;

with the proviso that when Y₁ is C₂₋₈alkylene and q is 0, then Y₄ is present; and

with the proviso that the compound is NOT:
2-cyano-1-[[4-[(4-phenylphenyl)sulfonylamino]phenyl]methyl]-3-(4-pyridyl)guanidine.

In some embodiments the present invention provides compounds of Formula IVa

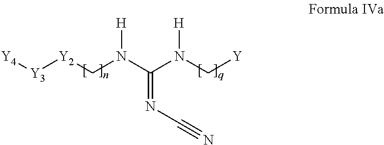

and pharmaceutically acceptable salts and solvates thereof;
wherein:

Y is 3-pyridinyl or 4-pyridinyl, optionally substituted as defined for Y for Formula I;

Y₂, Y₃, Y₄, and q are as defined for Formula IV above;

n is 3, 4, 5, 6, or 7; and

any methylene group of Y₂ and the n and q regions is optionally independently substituted with C₁₋₄alkyl, halo, C₁₋₄ haloalkyl, or C₃ or C₄ cycloalkyl.

In some embodiments the present invention provides compounds of Formula IVa1

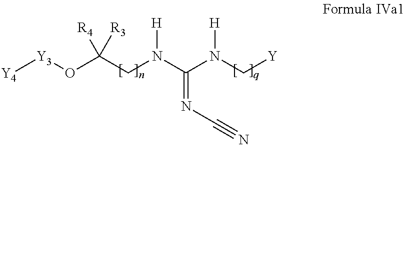

and pharmaceutically acceptable salts and solvates thereof;
wherein:

Y is as defined for Formula IVa above;

Y₃, Y₄, and q are as defined for Formula IV above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl; and

R₃ and R₄ are each independently H, halo, or C₁₋₄alkyl, or R₃ and R₄ taken together form a cyclopropyl or cyclobutyl ring.

In some embodiments the present invention provides compounds of Formula IVa2

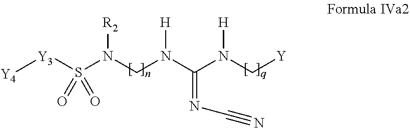

and pharmaceutically acceptable salts and solvates thereof;
wherein:

Y is as defined for Formula IVa above;

Y₃, Y₄, and q are as defined for Formula IV above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl; and

R₂ is H, halo, C₁₋₅alkyl, C₁₋₅alkenyl, or C₁₋₅alkynyl.

In some embodiments the present invention provides compounds of Formula IVa3

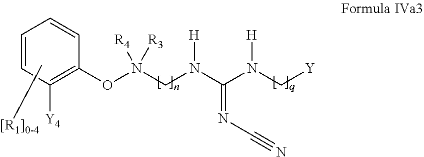

and pharmaceutically acceptable salts and solvates thereof;
wherein:

Y is as defined for Formula IVa above;

Y₄ and q are as defined for Formula IV above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl;

R₁, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₃ and R₄ are each independently H, halo, or C₁₋₄alkyl, or R₃ and R₄, taken together with the carbon to which they are attached, form a cyclopropyl or cyclobutyl ring.

In some embodiments the present invention provides compounds of Formula IVa4

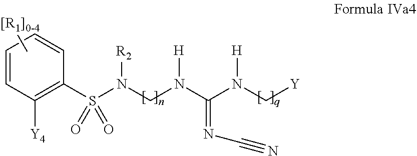

and pharmaceutically acceptable salts and solvates thereof;
wherein:

Y is as defined for Formula IVa above;

Y₄ and q are as defined for Formula IV above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₂ is H, halo, C₁₋₅alkyl, C₁₋₅alkenyl, or C₁₋₅alkynyl.

In some embodiments the present invention provides compounds of Formula IVa5

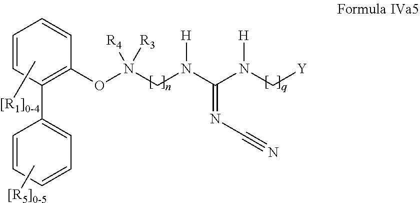

and pharmaceutically acceptable salts and solvates thereof;
wherein:

Y is as defined for Formula IVa above;

q is as defined for Formula IV above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₃ and R₄ are each independently H, halo, or C₁₋₄alkyl, or R₃ and R₄, taken together with the carbon to which they are attached, form a cyclopropyl or cyclobutyl ring.

In some embodiments the present invention provides compounds of Formula IVa6

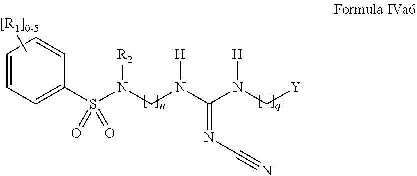

and pharmaceutically acceptable salts and solvates thereof;
wherein:

Y is as defined for Formula IVa above;

q is as defined for Formula IV above;

n is 3, 4, 5, 6, or 7;

any methylene group of the n and q regions is optionally independently substituted with C₁₋₄ alkyl, halo, C₁₋₄haloalkyl, or C₃ or C₄ cycloalkyl;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino; and

R₂ is H, halo, C₁₋₅alkyl, C₁₋₅alkenyl, or C₁₋₅alkynyl.

In some embodiments the present invention provides compounds of Formula IVb

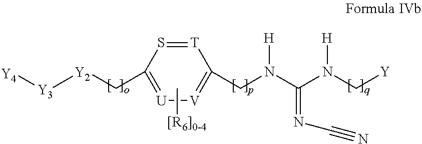

and pharmaceutically acceptable salts and solvates thereof;
wherein:

Y is 3-pyridinyl or 4-pyridinyl, optionally substituted as defined for Y for Formula I;

o, p, q, Y₂, Y₃, and Y₄ are as defined for Formula IV above;

any methylene group of the o, p, and q regions and Y₂ is optionally independently substituted with C₁₋₄alkyl, halo, C₁₋₄ haloalkyl, or C₃ or C₄ cycloalkyl;

R₆, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;

wherein S, T, U, and V are carbon or nitrogen, provided that when S, T, U, or V is nitrogen, then there is no substituent on the nitrogen;

with the proviso that when q is O, S, T, U, and V are carbon, and p is 1, then Y₄ is present; and

with the proviso that the compound is NOT 2-cyano-1-[[4-[(4-phenylphenyl) sulfonylamino]phenyl]methyl]-3-(4-pyridyl)guanidine.

In some embodiments the present invention provides compounds of Formula IVb1

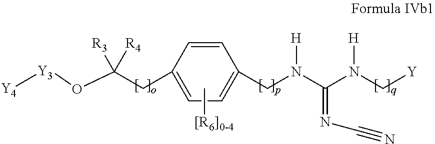

and pharmaceutically acceptable salts and solvates thereof;
wherein:

Y and R₆ are as defined for Formula IVb above;

o, p, q, Y₃, and Y₄ are as defined for Formula IV above;

any methylene group of the o, p, and q regions is optionally independently substituted with C₁₋₄alkyl, halo, C₁₋₄haloalkyl, or C₃ or C₄ cycloalkyl; and

R₃ and R₄ are each independently H, halo, or C₁₋₄alkyl, or R₃ and R₄, taken together with the carbon to which they are attached, form a cyclopropyl or cyclobutyl ring.

In some embodiments the present invention provides compounds of Formula IVb2

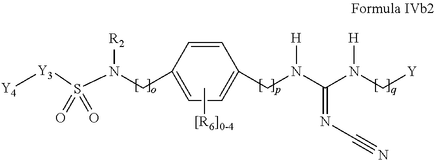

and pharmaceutically acceptable salts and solvates thereof;
wherein:

Y and R₆ are as defined for Formula IVb above;

o, p, q, Y₃, and Y₄ are as defined for Formula IV above;

any methylene group of the o, p, and q regions is optionally independently substituted with C₁₋₄alkyl, halo, C₁₋₄haloalkyl, or C₃ or C₄ cycloalkyl;

R₂ is H, halo, C₁₋₅alkyl, C₁₋₅alkenyl, or C₁₋₅alkynyl; and

with the proviso that the compound is NOT 2-cyano-1-[[4-[(4-phenylphenyl) sulfonylamino]phenyl]methyl]-3-(4-pyridyl)guanidine.

In some embodiments the present invention provides compounds of Formula IVb3

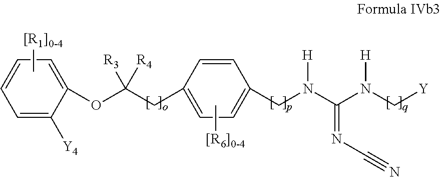

and pharmaceutically acceptable salts and solvates thereof;
wherein:

Y and R₆ are as defined for Formula IVb above;

o, p, q, and Y₄ are as defined for Formula IV above;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino;

R₃ and R₄ are each independently H, halo, or C₁₋₄alkyl, or R₃ and R₄, taken together with the carbon to which they are attached, form a cyclopropyl or cyclobutyl ring; and

any methylene group of the o, p, and q regions is optionally independently substituted with C₁₋₄alkyl, halo, C₁₋₄haloalkyl, or C₃ or C₄ cycloalkyl.

In some embodiments the present invention provides compounds of Formula IVb4

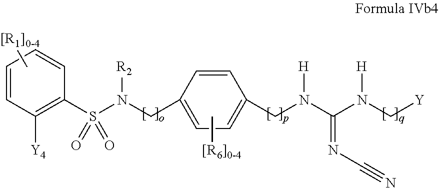

and pharmaceutically acceptable salts and solvates thereof;
wherein:

Y and R₆ are as defined for Formula IVb above;

o, p, q, and Y₄ are as defined for Formula IV above;

R₁, if present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino;

R₂ is H, halo, C₁₋₅alkyl, C₁₋₅alkenyl, or C₁₋₅alkynyl;

any methylene group of the o, p, and q regions is optionally independently substituted with C₁₋₄alkyl, halo, C₁₋₄haloalkyl, or C₃ or C₄ cycloalkyl.

In some embodiments the present invention provides compounds of Formula IVb5

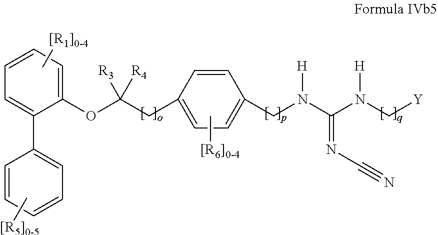

and pharmaceutically acceptable salts and solvates thereof;
wherein:

Y and R₆ are as defined for Formula IVb above;

o, p, and q are as defined for Formula IV above;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅alkoxy, C-amido, N-amido, amino, aminoalkyl, and alkylthio are each optionally substituted with heteroaryl, heterocyclo, cycloalkyl, alkoxy, or amino;

R₃ and R₄ are each independently H, halo, or C₁₋₄alkyl, or R₃ and R₄, taken together with the carbon to which they are attached, form a cyclopropyl or cyclobutyl ring; and

any methylene group of the o, p, and q regions is optionally independently substituted with C₁₋₄alkyl, halo, C₁₋₄haloalkyl, or C₃ or C₄ cycloalkyl.

In some embodiments the present invention provides compounds of Formula IVb6

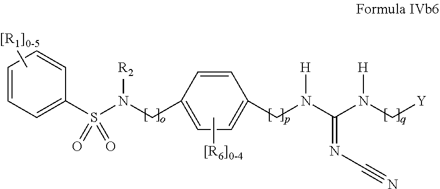

and pharmaceutically acceptable salts and solvates thereof;
wherein:

Y and R₆ are as defined for Formula IVb above;

o, p, and q are as defined for Formula IV above;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino;

R₂ is H, halo, C₁₋₅alkyl, C₁₋₅alkenyl, or C₁₋₅alkynyl; and

any methylene group of the o, p, and q regions is optionally independently substituted with C₁₋₄alkyl, halo, C₁₋₄haloalkyl, or C₃ or C₄ cycloalkyl.

In some embodiments the present invention provides compounds of Formula IVb7

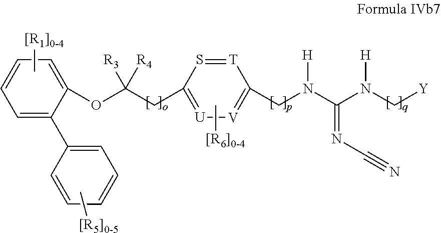

and pharmaceutically acceptable salts and solvates thereof;
wherein:

Y and R₆ are as defined for Formula IVa above;

o, p, and q are as defined for Formula IV above;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅alkoxy, C-amido, N-amido, amino, aminoalkyl, and alkylthio are each optionally substituted with heteroaryl, heterocyclo, cycloalkyl, alkoxy, or amino;

R₃ and R₄ are each independently H, halo, or C₁₋₄alkyl, or R₃ and R₄, taken together with the carbon to which they are attached, form a cyclopropyl or cyclobutyl ring;

any methylene group of the o, p, and q regions is optionally independently substituted with C₁₋₄alkyl, halo, C₁₋₄haloalkyl, or C₃ or C₄ cycloalkyl; and

S, T, U, and V are carbon or nitrogen, provided that at least one of S, T, U, and V is nitrogen and that when S, T, U, or V is nitrogen, then there is no substituent on the nitrogen.

In some embodiments the present invention provides compounds of Formula IVb8

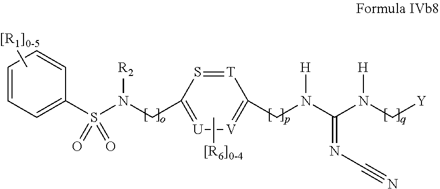

and pharmaceutically acceptable salts and solvates thereof;
wherein:

Y and R₆ are as defined for Formula IVb above;

o, p, and q are as defined for Formula IV above;

R₁, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heteroaryl, heterocyclo, cycloalkyl, alkoxy, or amino;

R₂ is H, halo, C₁₋₅ alkyl, C₁₋₅ alkenyl, or C₁₋₅ alkynyl;

any methylene group of the o, p, and q regions is optionally independently substituted with C₁₋₄alkyl, halo, C₁₋₄haloalkyl, or C₃ or C₄ cycloalkyl; and

S, T, U, and V are carbon or nitrogen, provided that at least one of S, T, U, and V is nitrogen and that when S, T, U, or V is nitrogen, then there is no substituent on the nitrogen.

In some embodiments the present invention provides compounds of Formula IVc

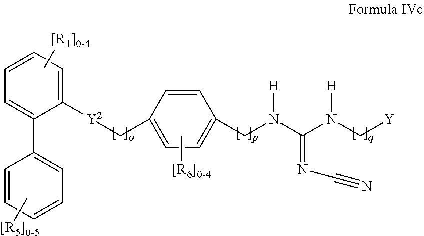

and pharmaceutically acceptable salts and solvates thereof;
wherein:

Y is 3-pyridinyl or 4-pyridinyl, optionally substituted as defined for Y for Formula I;

Y₂, o, p, and q are as defined for Formula IV;

R₁ and R₅, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅alkoxy, C-amido, N-amido, amino, aminoalkyl, and alkylthio are each optionally substituted with heteroaryl, heterocyclo, cycloalkyl, alkoxy, or amino;

R₆, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl; and any methylene group of the o, p, and q regions, or Y₂, is optionally independently substituted with C₁₋₄ alkyl, halo, C₁₋₄ haloalkyl, or C₃ or C₄ cycloalkyl; and with the proviso that when Y₂ is —C(═O)N(H)—, then Y₄ is present.

In some embodiments of the compounds of each of Formulae I, Ia, Ia1, Ia2, Ib, Ib1, Ib2, Ib3, Ic, and Id, Z₀ is carbocycle, cycloalkyl, cycloalkenyl, heterocycle, heterocyclonoyl, aryl, heteroaryl, carbocycloalkyl, heterocyclylalkyl, arylalkyl, arylalkenyl, heteroarylalkyl, heteroarylalkenyl, heteroarylalkynyl, or arylalkynyl, wherein each of the foregoing groups is substituted at least once with alkyl, alkylene, alkenyl, alkenylene, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide.

In some embodiments of the compounds of each of Formulae I, Ia, Ia1, Ia2, Ib, Ib1, Ib2, Ib3, Ic, and Id, Z₀ is selected from optionally substituted aryl, optionally substituted heteroaryl, optionally substituted carbocycle, and optionally substituted heterocycle.

In some embodiments of the compounds of each of Formulae I, Ia, Ia1, Ia2, Ib, Ib1, Ib2, Ib3, Ic, and Id, Z₀ is aryl optionally independently substituted one or more times with optionally substituted alkyl, N-amido, optionally substituted carbocycle, optionally substituted carbocycloamino, optionally substituted heterocycle, optionally substituted heterocycloalkyl, optionally substituted heterocycloamino, optionally substituted heterocyclonoyl, optionally substituted aryl, optionally substituted heteroaryl, halo, hydro, hydroxyl, optionally substituted hydroxyalkyl, optionally substituted haloalkoxy, optionally substituted alkoxy, optionally substituted aminoalkoxy, optionally substituted heterocycloalkoxy, optionally substituted haloalkyl, optionally substituted amino, optionally substituted aminoalkyl, nitro, optionally substituted C-amido, optionally substituted N-amido, cyano, or optionally substituted sulfonamide.

In some embodiments of the compounds of each of Formulae I, Ia, Ia1, Ia2, Ib, Ib1, Ib2, Ib3, Ic, and Id, Z₀ is a first aryl substituted with a second aryl, wherein each of the first aryl and the second aryl are optionally independently substituted one or more times with alkyl, N-amido, optionally substituted carbocycle, carbocycloamino, optionally substituted heterocycle, heterocycloalkyl, heterocycloamino, heterocyclonoyl, halo, hydro, hydroxyl, hydroxyalkyl, haloalkoxy, alkoxy, aminoalkoxy, heterocycloalkoxy, haloalkyl, optionally substituted amino, aminoalkyl, nitro, optionally substituted C-amido, optionally substituted N-amido, cyano, or sulfonamide. In some of such embodiments, the first aryl is phenyl. In some of such embodiments, the second aryl is phenyl. In some of such embodiments, the first aryl and the second aryl are both phenyl.

In some embodiments of the compounds of each of Formulae I, Ia, Ia1, Ia2, Ib, Ib1, Ib2, Ib3, Ic, and Id, Z₀ is optionally substituted phenyl, optionally substituted 2-pyridinyl, optionally substituted 3-pyridinyl, optionally substituted 4-pyridinyl, optionally substituted pyrimidine, optionally substituted pyrazine, optionally substituted pyrazole, optionally substituted thiophene, optionally substituted ortho-biphenyl, optionally substituted 1-naphthalenyl, optionally substituted 2-naphthalenyl, optionally substituted quinazoline, optionally substituted benzothiadiazine, optionally substituted indole, and optionally substituted pyridopyrimidine.

In some embodiments of the compounds of each of Formulae II, IIa, IIa1, IIa2, IIa3, IIa4, IIb, IIb1, IIb2, IIb3, IIb4, IIb5, IIb6, IIb7, IIc, IIc1, IId, and IId1, Z is hydro, alkyl, N-amido, optionally substituted carbocycle, carbocycloamino, optionally substituted heterocycle, heterocycloalkyl, heterocycloamino, heterocyclonoyl, optionally substituted aryl, optionally substituted heteroaryl, halo, hydro, hydroxyl, hydroxyalkyl, haloalkoxy, alkoxy, aminoalkoxy, heterocycloalkoxy, haloalkyl, optionally substituted amino, aminoalkyl, nitro, optionally substituted C-amido, optionally substituted N-amido, cyano, or sulfonamide.

In some embodiments of the compounds of each of Formulae II, IIa, IIa1, IIa2, IIa3, IIa4, IIb, IIb1, IIb2, IIb3, IIb4, IIb5, IIb6, IIb7, IIc, IIc1, IId, and IId1, Z is hydro, optionally substituted phenyl, optionally substituted pyridinyl, optionally substituted pyrimidine, optionally substituted pyrazole, optionally substituted piperidine, optionally substituted morpholine, optionally substituted piperazine, optionally substituted thiophene, optionally substituted imidazole, optionally substituted oxadiazole, optionally substituted oxazole, optionally substituted isoxazole, optionally substituted cyclohexyl, optionally substituted cyclohexylamino, optionally substituted piperidinylamino, or optionally substituted pyrrolidine.

In some embodiments of the compounds of each of Formulae IIa3, IIa4, IIb4, IIb5, IIb6, IIb7, IIc1, IId1, IIIa3, IIIa4, IIIa5, IIIa6, IIIb4, IIIb5, IIIb6, IIIb7, IIIb8, IIIb9, IIIb10, IIIb11, and IIIc, R₁ is not present, or is present one, two, three, or four times. In some embodiments of the compounds of each of Formulae IIIa6, IIIb8, and IIIb11, R₁ is present five times.

In some embodiments of the compounds of each of Formulae IIa3, IIa4, IIb4, IIb5, IIb6, IIb7, IIc1, IId1, IIIa3, IIIa4, IIIa5, IIIa6, IIIb4, IIIb5, IIIb6, IIIb7, IIIb8, IIIb9, IIIb10, IIIb11, IIIc, IVa3, IVa4, IVa5, IVb3, IVb4, IVb5, IVb7, and IVc, R₁ is an electron-withdrawing group, such as by way of non-limiting example, halo, trihalomethyl, nitro, cyano, C-carboxy, O-carboxy, C-amido, and N-amido.

In some embodiments of the compounds of each of Formulae IIIa4, IIIb5, IVa4, and IVb4, Y₄ is not present, R₁ is present two or three times, and each instance of R₁ is an electron-withdrawing group.

In some embodiments of the compounds of each of Formulae IIa3, IIa4, IIb4, IIb5, IIb6, IIb7, IIc1, IId1, IIIa3, IIIa4, IIIa5, IIIa6, IIIb4, IIIb5, IIIb6, IIIb7, IIIb8, IIIb9, IIIb1, IIIb11, IIIc, IVa3, IVa4, IVa5, IVb3, IVb4, IVb5, IVb7, and IVc, R₁ is selected from C₁₋₅ alkyl, C₁₋₅ alkoxy, C-amido, N-amido, amino, aminoalkyl, or alkylthio, each further substituted with heteroaryl, heterocyclo, cycloalkyl, alkoxy, or aminoheteroaryl, heterocyclo, cycloalkyl, alkoxy, or amino.

In some embodiments of the compounds of each of Formulae IIIa5, IIIb7, IIIb1, and IIIc, R₅ is not present or is present, one, two, three, four, or five times. In some embodiments of the compounds of each of Formulae IIIa5, IIIb7, IIIb8, IIIb9, IIIb1, IIIc, IVa5, IVb5, IVb7, and IVc, R₅ is selected from C₁₋₅ alkyl, C₁₋₅ alkoxy, C-amido, N-amido, amino, aminoalkyl, or alkylthio, each further substituted with heteroaryl, heterocyclo, cycloalkyl, alkoxy, or aminoheteroaryl, heterocyclo, cycloalkyl, alkoxy, or amino.

In some embodiments of the compounds of each of Formulae IIa3, IIa4, IIb4, IIb5, IIb6, IIb7, IIc1, IId1, IIIa3, IIIa4, IIIa5, IIIa6, IIIb4, IIIb5, IIIb6, IIIb7, IIIb8, IIIb9, IIIb1, IIIb11, IIIc, IVa3, IVa4, IVa5, IVb3, IVb4, IVb5, IVb7, and IVc, R₁ is selected from the following:

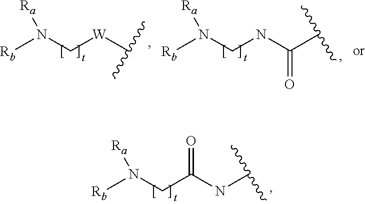

wherein t is 0, 1, 2, 3, or 4, W is N(H), O, C(H)₂, or S, and Ra and Rb are each independently hydro, C₃₋₆cycloalkyl, C₃₋₆heterocyclo (optionally substituted with C₁₋₅alkyl), or C₁₋₆alkyl, or Ra and Rb, together with the linking nitrogen between them, form azetidine (optionally substituted with morpholine, hydroxy, or C₁₋₅alkoxy), pyrrolidine, piperidine (optionally substituted with C₁₋₅alkyl), piperazine (optionally substituted with C₁₋₅alkyl), or morpholine.

In some embodiments of the compounds of each of Formulae IIIa5, IIIb7, IIIb8, IIIb9, IIIb1, IIIc, IVa5, IVb5, IVb7, and IVc, R₅ is selected from the following:

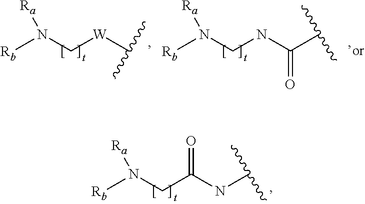

wherein t is 0, 1, 2, 3, or 4; W is N(H), O, C(H)₂, or S; and Ra and Rb are each independently hydro, C₃₋₆cycloalkyl, C₃₋₆heterocyclo (optionally substituted with C₁₋₅alkyl), or C₁₋₆alkyl, or Ra and Rb, together with the linking nitrogen between them, form azetidine (optionally substituted with morpholine, hydroxy, or C₁₋₅alkoxy), pyrrolidine, piperidine (optionally substituted with C₁₋₅alkyl), piperazine (optionally substituted with C₁₋₅alkyl), or morpholine.

In some embodiments of the compounds of each of Formulae IIIa5, IIIb7, IIIb8, IIIb9, IIIb10, IIIc, IVa5, IVb5, IVb7, and IVc, R₁ and/or R₅ is present and is located on the biphenyl ring as shown below:

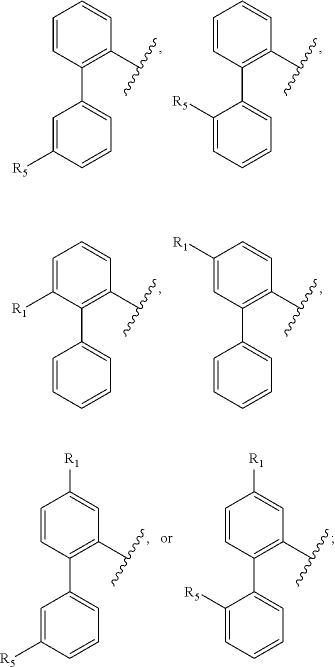

wherein R₁ and R₅ are each selected from the following:

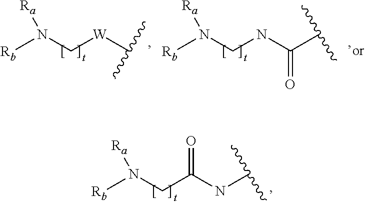

wherein t is 0, 1, 2, 3, or 4; W is N(H), O, C(H)₂, or S; and Ra and Rb are each independently hydro, C₃₋₆cycloalkyl, C₃₋₆heterocyclo (optionally substituted with C₁₋₅alkyl), or C₁₋₆alkyl, or Ra and Rb, together with the linking nitrogen between them, form azetidine (optionally substituted with morpholine, hydroxy, or C₁₋₅alkoxy), pyrrolidine, piperidine (optionally substituted with C₁₋₅alkyl), piperazine (optionally substituted with C₁₋₅ alkyl), or morpholine; with the proviso that when R₁ and R₅ are both present on the biphenyl ring, then R₁ is C₁₋₄ haloalkyl (such as, for example, trifluoromethyl) or halo (such as, for example, chloro).

In some embodiments of the compounds of each of Formulae Ia2, Ib2, Id, IIa2, IIa4, IIb2, IIb5, IId, IId1, IIIa2, IIIa4, IIIa6, IIIb2, IIIb5, IIIb51IIb8, IIIb11, IVa2, IVa4, IVa6, IVb2, IVb4, IVb6, and IVb8, R₂ is hydrogen or cyclopropyl. In some of such embodiments, R₂ is hydrogen.

In some embodiments of the compounds of each of Formulae I, II, III, and IV, R for the purposes of Y is hydrogen.

In some embodiments of the compounds of each of Formulae I, II, III, and IV, R for the purposes of Y₁ is hydrogen.

In some embodiments of the compounds of each of Formulae I, II, III, and IV, R for the purposes of Y₂ is hydrogen.

In some embodiments of the compounds of each of Formulae Ibl, Ic, IIb1, IIb4, IIc, Ilcl, IIIa1, IIIa3, IIIa5, IIIb1, IIIb4, IIIb7, IIIb8, IIIb9, IIIb10, IIIc, IVa1, IVa3, IVa5, IVb1, IVb3, IVb5, and IVb7, R₃ and R₄ are both hydrogen or both fluoro. In some of such embodiments, R₃ and R₄ are both hydrogen.

In some embodiments of the compounds of each of Formulae Ib, Ib1, Ib2, Ib3, IIb, IIb1, IIb2, IIb3, IIb4, IIb5, IIb6, IIb7, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, R₆ is not present, or is present one, two, three, or four times. In some of such embodiments R₆, is not present or is fluoro, methyl, or trifluormethyl. In some of such embodiments R₆ is not present.

In some embodiments of the compounds of each of Formulae Ia, Ia1, Ia2, IIa, IIa1, IIa2, IIa3, IIa4, IIIa, IIa1, IIIa2, IIIa3, IIIa4, IIIa5, IIIa6, IVa, IVa1, IVa2, Iva3, IVa4, IVa5, and IVa6, n is 4, 5, or 6. In some embodiments of the compounds of each of Formulae Ia, Ia1, Ia2, IIa, IIa1, IIa2, IIa3, IIa4, IIIa, IIa1, IIIa2, IIIa3, IIIa4, IIIa5, IIIa6, IVa, IVa1, IVa2, Iva3, IVa4, IVa5, and IVa6, n is 4. In some embodiments of the compounds of each of Formulae Ia, Ia1, Ia2, IIa, IIa1, IIa2, IIa3, IIa4, IIIa, IIa1, IIIa2, IIIa3, IIIa4, IIIa5, IIIa6, IVa, IVa1, IVa2, Iva3, IVa4, IVa5, and IVa6, n is 5. In some embodiments of the compounds of each of Formulae Ia, Ia1, Ia2, IIa, IIa1, IIa2, IIa3, IIa4, IIIa, IIa1, IIIa2, IIIa3, IIIa4, IIIa5, IIIa6, IVa, IVa1, IVa2, Iva3, IVa4, IVa5, and IVa6, n is 6. In some embodiments of the compounds of each of Formulae Ia, Ia1, Ia2, IIa, IIa1, IIa2, IIa3, IIa4, IIIa, IIa1, IIIa2, IIIa3, IIIa4, IIIa5, IIIa6, IVa, IVa1, IVa2, Iva3, IVa4, IVa5, and IVa6, any methylene groups of the n region are optionally substituted with fluoro or methyl. In some embodiments of the compounds of each of Formulae Ia, Ia1, Ia2, IIa, IIa1, IIa2, IIa3, IIa4, IIIa, IIa1, IIIa2, IIIa3, IIIa4, IIIa5, IIIa6, IVa, IVa1, IVa2, Iva3, IVa4, IVa5, and IVa6, any methylene groups of the n region are all fully saturated.

In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, o is 0. In some embodiments of the compounds of each of Formulae IIIIII, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, o is 1. In some embodiments of the compounds of each of Formulae III, IIIb, IIIb1, IIIb2, IIIb3, IIIb4, IIIb5, IIIb6, IIIb7, IIIb8, IIIb9, IIIb10, IIIb1, IIIc, IVb, IVb1, IVb2, IVb3, IVb4, IVb5, IVb6, IVb7, IVb8, and IVc, o is 2. In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, any methylene groups of the o region are optionally substituted with fluoro or methyl. In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, any methylene groups of the o region are all fully saturated.

In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, p is 0. In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, p is 1. In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, p is 2. In some embodiments of the compounds of each of Formulae III, IIIb, IIIb1, IIIb2, IIIb3, IIIb4, IIIb5, IIIb6, IIIb7, IIIb8, IIIb9, IIIb1, IIIb11, IIIc, IVb, IVb1, IVb2, IVb3, IVb4, IVb5, IVb6, IVb7, IVb8, and IVc, any methylene groups of the p region are optionally substituted with fluoro or methyl. In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IVb, IVb1, IVb2, IVb3, IVb4, IVb5, IVb6, IVb7, IVb8, and IVc, any methylene groups of the p region are all fully saturated.

In some embodiments of the compounds of each of Formulae III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, q is 0. In some embodiments of the compounds of each of Formulae III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, q is 1. In some embodiments of the compounds of each of Formulae III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, q is 2. In some embodiments of the compounds of each of Formulae III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, any methylene groups of the q region are optionally substituted with fluoro or methyl. In some embodiments of the compounds of each of Formulae III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, any methylene groups of the q region are all fully saturated.

In some embodiments of the compounds of each of Formulae Ib3, IIb3, IIb6, IIIb3, IIIb6, and IIIb9, u is 0. In some embodiments of the compounds of each of Formulae Ib3, IIb3, IIb6, IIIb3, IIIb6, and IIIb9, u is 1. In some embodiments of the compounds of each of Formulae Ib3, IIb3, IIb6, IIIb3, IIIb6, and IIIb9, when u is 1, then the methylene group of the u region is substituted with fluoro or methyl. In some embodiments of the compounds of each of Formulae Ib3, IIb3, IIb6, IIIb3, IIIb6, and IIIb9, when u is 1, then the methylene group of the u region is fully saturated.

In some embodiments of the compounds of each of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, and IId1, any methylene groups are all fully saturated.

In some embodiments of the compounds of each of Formulae I, II, III, and IV, Y is phenyl. In some embodiments of the compounds of each of Formulae I, II, III, and IV, Y is 2-pyridinyl. In some of either of such embodiments, Y is not substituted or is substituted one, two, three, or four times as defined for Y for Formula I and II. Furthermore, in some of such embodiments, any substituent of Y is halo (such as, for example, fluoro), methyl, nitro, cyano, trihalomethyl, methoxy, amino, hydroxyl, or mercapto.

In some embodiments of the compounds of each of Formulae I, II, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, Y is 3-pyridinyl. In some embodiments of the compounds of each of Formulae I, II, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, Y is 4-pyridinyl. In some embodiments of the compounds of each of Formulae I, II, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, Y is not substituted or is substituted one, two, three, or four times as defined for Y for Formula I. In some embodiments of the compounds of each of Formulae I, II, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, any substitutent of Y is halo (such as, for example, fluoro), methyl, nitro, cyano, trihalomethyl, methoxy, amino, hydroxyl, or mercapto. In some embodiments of the compounds of each of Formulae I, II, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, Y is unsubstituted 3-pyridinyl or is 3-pyridinyl substituted at the 4 position with NH₂.

In some embodiments of the compounds of each of Formulae II, IIa, IIa2, IIb, IIb2, and IId, Z and/or any substituents on Y₃ are selected so that Y₃ is an electron-deficient aryl or heteroaryl ring.

In some embodiments of the compounds of each of Formulae IIa4, IIb5, and IId1, Z and/or R₁ are selected so that the phenyl ring is electron deficient.

In some embodiments of the compounds of each of Formulae III, IIIa, IIIa2, IIIb, IIIb2, IV, IVa, IVa2, IVb, and IVb2, Y₄ is not present and any substituents on Y₃ are selected so that Y₃ is electron-deficient.

In some embodiments of the compounds of each of Formulae I, Ic, Id, II, IIc, IIc1, IId, IId1, III, and IV, Y₁ is divalent carbocycle, divalent heterocycle, divalent phenyl or divalent heteroaryl, wherein any ring carbon atom is optionally independently substituted with halo, C₁₋₅ alkyl, nitro, cyano, trihalomethyl, C₁₋₅ alkoxy, C-amido, N-amido, sulfonamide, amino, aminosulfonyl, hydroxyl, mercapto, alkylthio, sulfonyl, or sulfinyl.

In some embodiments of the compounds of each of Formulae I, Ic, Id, II, IIc, IIc1, IId, IId1, III, and IV, Y₁ is divalent cyclohexyl, divalent piperidinyl, divalent phenyl, divalent pyridinyl, divalent pyrimidinyl, divalent thiophenyl, and divalent triazolyl, wherein any ring carbon is optionally further independently substituted with halo, C₁₋₅ alkyl, nitro, cyano, trihalomethyl, C₁₋₅ alkoxy, C-amido, N-amido, sulfonamide, amino, aminosulfonyl, hydroxyl, mercapto, alkylthio, sulfonyl, or sulfinyl.

In some embodiments of the compounds of each of Formulae I, Ia, Ib, II, IIa, IIb, IIb7, III, IIIa, IIIb, IIIc, IV, IVa, IVb, and IVc, Y₂ is —OCH₂—, —SCH₂—, —N(R)CH₂—, —CH₂O—, —CH₂S—, —CH₂N(R)—, —SO₂N(R)—, —N(R)SO₂—, —C₁₋₄alkylene-SO₂N(R)—, —C₁₋₄alkylene-N(R)SO₂—, —SO₂N(R)—C₁₋₄alkylene-, —N(R)SO₂—C₁₋₄ alkylene-, —C₁₋₄alkylene-O—C₁₋₄ alkylene-, —O—C₁₋₄ alkylene-, —C₁₋₄ alkylene-O—, —S—C₁₋₄ alkylene-, —C₁₋₄alkylene-S—, —C₁₋₄alkylene-S—C₁₋₄ alkylene-, —N(R)—C₁₋₄ alkylene-, —C₁₋₄alkylene-N(R)—, or —C₁₋₄alkylene-N(R)—C₁₋₄ alkylene-, wherein R is H, halo, C₁₋₅ alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —S(═O)₂CH₂—, —S(═O)CH₂—, —CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, —O—C₁₋₄ alkylene-N(R)C(═O)—, —C₁₋₄alkylene-S(═O)₂—, —C₁₋₄alkylene-S(═O)—, —S(═O)₂—C₁₋₄alkylene-, —S(═O)—C₁₋₄ alkylene-, —C₁₋₄alkylene-SO₂N(R)—, —C₁₋₄alkylene-N(R)SO₂—, —SO₂N(R)—C₁₋₄ alkylene-, —N(R)SO₂—C₁₋₄ alkylene-, —C₁₋₄alkylene-O—C₁₋₄ alkylene-, —O—C₁₋₄ alkylene-, —C₁₋₄alkylene-O—, —C₁₋₄ alkylene-S—, —C₁₋₄alkylene-S—C₁₋₄ alkylene-, —C₁₋₄alkylene-N(R)—, —C₁₋₄alkylene-N(R)—C₁₋₄ alkylene-, —C₁₋₄alkylene-C(═O)—O—C₁₋₄alkylene-, —C₁₋₄alkylene-O—C(═O)—C₁₋₄alkylene-, —C₁₋₄alkylene-C(═O)—N(R)—C₁₋₄ alkylene-, or —C₁₋₄alkylene-N(R)—C(═O)—C₁₋₄alkylene-, wherein R is H, halo, C₁₋₅ alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —SCH₂—.

In some embodiments of the compounds of each of Formulae I, Ia, Ib, II, IIa, IIb, IIb7, III, IIIa, IIIb, IIIc, IV, IVa, IVb, and IVc, Y₂ is —N(R)CH₂—, wherein R is H, halo, C₁₋₅alkyl, C₁₋₅ alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —N(R)C(═O)—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C(═O)N(R)—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —S(═O)₂CH₂—.

In some embodiments of the compounds of each of Formulae I, Ia, Ib, II, IIa, IIb, IIb7, III, IIIa, IIIb, IIIc, IV, IVa, IVb, and IVc, Y₂ is —S(═O)CH₂—.

In some embodiments of the compounds of each of Formulae I, Ia, Ib, II, IIa, IIb, IIb7, III, IIIa, IIIb, IIIc, IV, IVa, IVb, and IVc, Y₂ is —CH₂S—.

In some embodiments of the compounds of each of Formulae I, Ia, Ib, II, IIa, IIb, IIb7, III, IIIa, IIIb, IIIc, IV, IVa, IVb, and IVc, Y₂ is —CH₂N(R)—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —CH₂S(═O)₂—.

In some embodiments of the compounds of each of Formulae I, Ia, Ib, II, IIa, IIb, IIb7, III, IIIa, IIIb, IIIc, IV, IVa, IVb, and IVc, Y₂ is —CH₂S(═O)—.

In some embodiments of the compounds of each of Formulae I, Ia, Ib, II, IIa, IIb, IIb7, III, IIIa, IIIb, IIIc, IV, IVa, IVb, and IVc, Y₂ is —C(═O)O—.

In some embodiments of the compounds of each of Formulae I, Ia, Ib, II, IIa, IIb, IIb7, III, IIIa, IIIb, IIIc, IV, IVa, IVb, and IVc, Y₂ is —OC(═O)—.

In some embodiments of the compounds of each of Formulae I, Ia, Ib, II, IIa, IIb, IIb7, III, IIIa, IIIb, IIIc, IV, IVa, IVb, and IVc, Y₂ is —N(R)SO₂—, wherein R is H, halo, C₁₋₅ alkyl, C₁₋₅ alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is ethylene.

In some embodiments of the compounds of each of Formulae I, Ia, Ib, II, IIa, IIb, IIb7, III, IIIa, IIIb, IIIc, IV, IVa, IVb, and IVc, Y₂ is propylene.

In some embodiments of the compounds of each of Formulae I, Ia, Ib, II, IIa, IIb, IIb7, III, IIIa, IIIb, IIIc, IV, IVa, IVb, and IVc, Y₂ is n-butylene.

In some embodiments of the compounds of each of Formulae I, Ia, Ib, II, IIa, IIb, IIb7, III, IIIa, IIIb, IIIc, IV, IVa, IVb, and IVc, Y₂ is —O—C₁₋₄ alkylene-N(R)C(═O)—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —O—C₁₋₄ alkylene-C(═O)N(R)—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —N(R)C(═O)—C₁₋₄ alkylene-O—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C(═O)N(R)—C₁₋₄ alkylene-O—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄alkylene-S(═O)₂—.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄ alkylene-S(═O)—.

In some embodiments of the compounds of each of Formulae I, Ia, Ib, II, IIa, IIb, IIb7, III, IIIa, IIIb, IIIc, IV, IVa, IVb, and IVc, Y₂ is —S(═O)₂—C₁₋₄ alkylene-.

In some embodiments of the compounds of each of Formulae I, Ia, Ib, II, IIa, IIb, IIb7, III, IIIa, IIIb, IIIc, IV, IVa, IVb, and IVc, Y₂ is —S(═O)—C₁₋₄ alkylene-.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄ alkylene-SO₂N(R)—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄ alkylene-N(R)SO₂—, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —SO₂N(R)—C₁₋₄ alkylene-, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —N(R)SO₂—C₁₋₄ alkylene-, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄ alkylene-O—C₁₋₄ alkylene-.

In some embodiments of the compounds of each of Formulae I, Ia, Ib, II, IIa, IIb, IIb7, III, IIIa, IIIb, IIIc, IV, IVa, IVb, and IVc, Y₂ is —O—C₁₋₄ alkylene-.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄ alkylene-O—.

In some embodiments of the compounds of each of Formulae I, Ia, Ib, II, IIa, IIb, IIb7, III, IIIa, IIIb, IIIc, IV, IVa, IVb, and IVc, Y₂ is —S—C₁₋₄alkylene-.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄alkylene-S—.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄ alkylene-S—C₁₋₄ alkylene-.

In some embodiments of the compounds of each of Formulae I, Ia, Ib, II, IIa, IIb, IIb7, III, IIIa, IIIb, IIIc, IV, IVa, IVb, and IVc, Y₂ is —N(R)—C₁₋₄ alkylene-, wherein R is H, halo, C₁₋₅ alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄alkylene-N(R)—, wherein R is H, halo, C₁₋₅ alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄alkylene-N(R)—C₁₋₄ alkylene-, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄alkylene-C(═O)—O—C₁₋₄alkylene-.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄alkylene-O—C(═O)—C₁₋₄alkylene-.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄alkylene-C(═O)—N(R)—C₁₋₄alkylene-, wherein R is H, halo, C₁₋₅alkyl, C₁₋₅alkenyl, or C₁₋₅alkynyl.

In some embodiments of the compounds of each of Formulae I, Ia, Ib, II, IIa, IIb, IIb7, III, IIIa, IIIb, IIIc, IV, IVa, IVb, and IVc, Y₂ is —C₁₋₄alkylene-N(R)—C(═O)—C₁₋₄alkylene-, wherein R is H, halo, C₁₋₅alkyl, C₁₋₅alkenyl, or C₁₋₅alkynyl.

In some embodiments of the compounds of each of Formulae II, IIa, IIa1, IIa2, IIb, IIb1, IIb2, IIb3, IIc, IId, III, IIIa, IIIa1, IIIa2, IIIb, IIIb1, IIIb2, IIIb3, IV, IVa, IVa1, IVa2, IVb, IVb1, and IVb2, Y₃ is phenyl, pyridinyl, pyrimidinyl, divalent phenyl, divalent pyridinyl, or divalent pyrimidinyl, wherein any ring carbon is optionally independently substituted, and in the case of divalent rings, optionally further independently substituted, with halo, C₁₋₅ alkyl, nitro, cyano, trihalomethyl, C₁₋₅alkoxy, C-amido, N-amido, sulfonamide, amino, aminosulfonyl, hydroxyl, mercapto, alkylthio, sulfonyl, or sulfinyl, wherein C₁₋₅ alkyl, C₁₋₅ alkoxy, C-amido, N-amido, amino, and alkylthio are each optionally substituted with heteroaryl, heterocyclo, cycloalkyl, alkoxy, or aminoheteroaryl, heterocyclo, cycloalkyl, alkoxy, or amino.

In some embodiments of the compounds of each of Formulae III, IIIa, IIIa1, IIIa2, IIIa3, IIIa4, IIIb, IIIb1, IIIb2, IIIb3, IIIb4, IIIb5, IIIb6, IV, IVa, IVa1, IVa2, Iva3, IVa4, IVb, IVb1, IVb2, IVb3, and IVb4, Y₄ is optionally present, and when present is aryl, heteroaryl, carbocycle, or heterocycle, wherein any ring carbon atom is optionally independently substituted with halo, C₁₋₅ alkyl, nitro, cyano, trihalomethyl, C₁₋₅ alkoxy, C-amido, N-amido, sulfonamide, amino, aminosulfonyl, hydroxyl, mercapto, alkylthio, sulfonyl, sulfinyl, wherein C₁₋₅ alkyl, C₁₋₅ alkoxy, C-amido, N-amido, amino, and alkylthio are each optionally substituted with heteroaryl, heterocyclo, cycloalkyl, alkoxy, or aminoheteroaryl, heterocyclo, cycloalkyl, alkoxy, or amino.

In some embodiments of the compounds of each of Formulae III, IIIa, IIIa1, IIIa2, IIIa3, IIIa4, IIIb, IIIb1, IIIb2, IIIb3, IIIb4, IIIb5, IIIb6, IV, IVa, IVa1, IVa2, Iva3, IVa4, IVb, IVb1, IVb2, IVb3, and IVb4, Y₄ is present.

In some embodiments of the compounds of each of Formulae III, IIIa, IIIa1, IIIa2, IIIa3, IIIa4, IIIb, IIIb1, IIIb2, IIIb3, IIIb4, IIIb5, IIIb6, IV, IVa, IVa1, IVa2, Iva3, IVa4, IVb, IVb1, IVb2, IVb3, and IVb4, Y₄ is a group selected from phenyl, morpholino, piperazinyl, oxidiazolyl, oxazolyl, pyrrolidinyl, thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl (such as, for example, 2H-pyrrolyl), pyrroline, imidazolyl, imidazolidinyl, pyrazolyl, pyridyl (pyridinyl) (such as, for example, 2-pyridyl, 3-pyridyl, and 4-pyridyl), pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, thiazolyl, phenothiazinyl, isoxazolyl, furazanyl, phenoxazinyl, 1,4-dihydroquinoxaline-2,3-dione, 7-amino-isocoumarin, pyrido[1,2-a]pyrimidin-4-one, pyrazolo[1,5-a]pyrimidinyl (such as, for example, pyrazolo[1,5-a]pyrimidin-3-yl), 1,2-benzoisoxazo 1-3-yl, benzimidazolyl, 2-oxindolyl, 2-oxobenzimidazolyl, triazine, dioxoanyl, dithianyl, thiomorpholinyl, trithianyl, cyclobutyl, cyclohexyl, cycloheptyl, cyclooctyl, and cyclohexenyl, wherein each of the groups is optionally substituted as defined for Y₄ in Formula III.

In some embodiments of the compounds of each of Formulae III, IIIa, IIIa1, IIIa2, IIIa3, IIIa4, IIIb, IIIb1, IIIb2, IIIb3, IIIb4, IIIb5, IIIb6, IV, IVa, IVa1, IVa2, Iva3, IVa4, IVb, IVb1, IVb2, IVb3, and IVb4, Y₄ is a group selected from phenyl, 2-pyridinyl, 3-pyridinyl, 4-pyridinyl, pyrimidinyl, morpholino, piperazinyl, oxidiazolyl, oxazolyl, pyrrolidinyl, imidazolyl, and piperidinyl, wherein each of the groups is optionally substituted as defined for Y₄ in Formula III.

In some embodiments of the compounds of each of Formulae III, IIIa, IIIa1, IIIa2, IIIa3, IIIa4, IIIb, IIIb1, IIIb2, IIIb3, IIIb4, IIIb5, IIIb6, IV, IVa, IVa1, IVa2, Iva3, IVa4, IVb, IVb1, IVb2, IVb3, and IVb4, Y₄ is a group selected from:

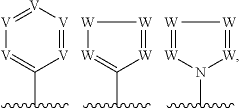

wherein V is N or C(H) and W is N, O, C(H), or S, wherein any ring atom is optionally independently substituted with halo, C₁₋₅alkyl, nitro, cyano, trihalomethyl, C₁₋₅ alkoxy, C-amido, N-amido, sulfonamide, amino, aminosulfonyl, hydroxyl, mercapto, alkylthio, sulfonyl, sulfinyl, wherein C₁₋₅ alkyl, C₁₋₅ alkoxy, C-amido, N-amido, amino, and alkylthio are each optionally substituted with heteroaryl, heterocyclo, cycloalkyl, alkoxy, or aminoheteroaryl, heterocyclo, cycloalkyl, alkoxy, or amino.

In some embodiments of the compounds of each of Formulae Ib, IIb, IIIb, IIIb10, IIIb11, IIIc, IVb, IVb7, IVb8, and IVc, at least two of S, T, U, and V are nitrogen. In some embodiments of the compounds of each of Formulae Ib, IIb, IIIb, IIIb1, IIIb11, IIIc, IVb, IVb7, IVb8, and IVc, only S is nitrogen. In some embodiments of the compounds of each of Formulae Ib, IIb, IIIb, IIIb1, IIIb11, IIIc, IVb, IVb7, IVb8, and IVc, only T is nitrogen. In some embodiments of the compounds of each of Formulae Ib, IIb, IIIb, IIIb10, IIIb11, IIIc, IVb, IVb7, IVb8, and IVc, only U is nitrogen. In some embodiments of the compounds of each of Formulae Ib, IIb, IIIb, IIIb10, IIIb11, IIIc, IVb, IVb7, IVb8, and IVc, only V is nitrogen. In some embodiments of the compounds of each of Formulae Ib, IIb, IIIb, IIIb1, IIIb11, IIIc, IVb, IVb7, IVb8, and IVc, T and V are nitrogen. In some embodiments of the compounds of each of Formulae Ib, IIb, IIIb, IIIb10, IIIb11, IIIc, IVb, IVb7, IVb8, and IVc, S and U are nitrogen.

In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IV, IVb, IVb1, IVb2, IVb3, IVb4, IVb5, IVb6, IVb7, IVb8, and IVc, Y is unsubstituted 3-pyridinyl and q is 1.

In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IV, IVb, IVb1, IVb2, IVb3, IVb4, IVb5, IVb6, IVb7, IVb8, and IVc, Y is unsubstituted 3-pyridinyl, q is 1, and p is 0.

In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IV, IVb, IVb1, IVb2, IVb3, IVb4, IVb5, IVb6, IVb7, IVb8, and IVc, Y is unsubstituted 3-pyridinyl, q is 1, p is 0, and o is 0.

In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IV, IVb, IVb1, IVb2, IVb3, IVb4, IVb5, IVb6, IVb7, IVb8, and IVc, Y is unsubstituted 3-pyridinyl, q is 1, p is 0, and o is 0.

In some embodiments of the compounds of each of Formulae III, IIIb, IIIb1, IIIb2, IIIb3, IIIb4, IIIb5, IIIb6, IIIb7, IIIb8, IIIb9, IIIb10, IIIb11, IIIc, IV, IVb, IVb1, IVb2, IVb3, IVb4, IVb5, IVb6, IVb7, IVb8, and IVc, Y is unsubstituted 3-pyridinyl, q is 1, p is 0, o is 0, and R₆ is not present.

In some embodiments of the compounds of each of Formulae III, IIIa, IIIa1, IIIa2, IIIa3, IIIa4, IIIa5, IIIa6, IV, IVa, IVa1, IVa2, Iva3, IVa4, IVa5, and IVa6, Y is unsubstituted 3-pyridinyl and q is 1.

In some embodiments of the compounds of each of Formulae III, IIIa, IIIa1, IIIa2, IIIa3, IIIa4, IIIa5, IIIa6, IV, IVa, IVa1, IVa2, Iva3, IVa4, IVa5, and IVa6, Y is unsubstituted 3-pyridinyl, q is 1, and n is 4, 5, or 6.

In some embodiments of the compounds of each of Formulae III, IIIa, IIIa1, IIIa2, IIIa3, IIIa4, IIIa5, IIIa6, IV, IVa, IVa1, IVa2, Iva3, IVa4, IVa5, and IVa6, Y is unsubstituted 3-pyridinyl, q is 1, n is 4, 5, or 6, and the methylene groups of n and q are all fully saturated.

In some embodiments of the compounds of each of Formulae Ib, Ib1, Ib2, Ib3, IIb, IIb1, IIb2, IIb3, IIb4, IIb5, IIb6, and IIb7, R₆ and R₇ are not present.

In some embodiments of the compounds of each of Formulae Ib, Ib1, Ib2, Ib3, IIb, IIb1, IIb2, IIb3, IIb4, IIb5, IIb6, and IIb7, R₆ and R₇ are not present and any methylene groups are fully saturated.

In some embodiments of the compounds of each of Formulae Ia, Ia1, Ia2, IIa, IIa1, IIa2, IIa3, and IIa4, n is 4, 5, or 6, and R₇ is not present.

In some embodiments of the compounds of each of Formulae Ia, Ia1, Ia2, IIa, IIa1, IIa2, IIa3, and IIa4, n is 4, 5, or 6, R₇ is not present, and any methylene groups are fully saturated.

The compounds of the present invention include the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, as well as for any of the foregoing their stereochemically isomeric forms thereof. The compounds of the present invention also include pharmaceutically acceptable salts, prodrugs, N-oxide forms, quaternary amines, and solvates of the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6.

For therapeutic use, salts of the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, are those particular salts wherein the counterion is pharmaceutically acceptable. However, salts of acids and bases which are non-pharmaceutically acceptable can also find use, for example, in the preparation or purification of a pharmaceutically acceptable compound. All salts, whether pharmaceutically acceptable or not, are within the ambit of the present invention.

The pharmaceutically acceptable addition salts as mentioned herein are meant to comprise the therapeutically active non-toxic acid addition salt forms which the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, are able to form. The salts can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, containing acidic protons can be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanedi-ol, hydrabamine salts, and salts with amino acids such as, for example, arginine, lysine and the like. Conversely the salt form can be converted by treatment with acid into the free acid form.

The term addition salt also comprises the hydrates and solvent addition forms which the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, are able to form. Examples of such forms are e.g. hydrates, alcoholates and the like.

The term “quaternary amine” as used herein defines the quaternary ammonium salts which the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, are able to form by reaction between a basic nitrogen of one of the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and an appropriate quaternizing agent, such as, for example, an optionally substituted alkylhalide, arylhalide or arylalkylhalide, e.g. methyliodide or benzyliodide. Other reactants with good leaving groups can also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

Pharmaceutically acceptable salts of the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, include all salts are exemplified by alkaline salts with an inorganic acid and/or a salt with an organic acid that are known in the art. In addition, pharmaceutically acceptable salts include acid salts of inorganic bases, as well as acid salts of organic bases. Their hydrates, solvates, and the like are also encompassed in the present invention. In addition, N-oxide compounds are also encompassed in the present invention.

It will be appreciated that some of the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and their N-oxides, addition salts, quaternary amines and stereochemically isomeric forms can contain one or more centers of chirality and exist as stereochemically isomeric forms.

The term “stereochemically isomeric forms” as used hereinbefore defines all the possible stereoisomeric forms which the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and their N-oxides, addition salts, quaternary amines or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIIb1, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and their N-oxides, salts, solvates or quaternary amines substantially free, i.e. associated with less than 10%, preferably less than 5%, in particular less than 2% and most preferably less than 1% of the other isomers. In particular, stereogenic centers can have the R- or S-configuration; substituents on bivalent cyclic (partially) saturated radicals can have either the cis- or trans-configuration. Compounds encompassing double bonds can have an E or Z-stereochemistry at said double bond. Stereochemically isomeric forms of the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, are fully intended to be embraced within the scope of this invention.

“N-oxides” are meant to comprise the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, wherein one or several nitrogen atoms are oxidized to the so-called N-oxide.

Some of the compounds of Formulae I, Ia, Ia1, Ia2, Ib, Ib1, Ib2, Ib3, Ic, Id, II, IIa, IIIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can also exist in their tautomeric form. Such forms although not explicitly indicated in the above formula are intended to be included within the scope of the present invention.

In preferred embodiments, compounds of the present invention are provided having an IC₅₀ of less than about 100 nM, such as, for example, the compounds listed in Tables 1A and 1B and 3A and 3B, as determined in the cytotoxicity assays as described in the Examples below (i.e., Cytotoxicity Assays).

In all compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb100, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, reference to any bound hydrogen atom can also encompass a deuterium atom bound at the same position. Substitution of hydrogen atoms with deuterium atoms is conventional in the art. See, e.g., U.S. Pat. Nos. 5,149,820 & 7,317,039, which are incorporated by reference herein their entirety. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms slows the catabolism of the deuterated compound, relative to the non-deuterated compound, such that the deuterated compound exhibits a longer half-life in the bodies of individuals administered such compounds. This particularly so when the catabolism of the hydrogenated compound is mediated by cytochrome P450 systems. See Kushner et al., Can. J. Physiol. Pharmacol. (1999) 77:79-88, which is incorporated by reference herein its entirety.

### 3. Pharmaceutical Compositions and Formulations

In another aspect, the present invention further provides a composition for use as a medicament or a pharmaceutical composition comprising one of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and a pharmaceutically-acceptable excipient. In some of such embodiments, the medicament or pharmaceutical composition comprises a therapeutically or prophylactically effective amount of at least one of the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6.

In some of such embodiments, the composition or pharmaceutical composition is for use in treating cancer, systemic or chronic inflammation, rheumatoid arthritis, diabetes, obesity, T-cell mediated autoimmune disease, ischemia, and other complications associated with these diseases and disorders. In some of such embodiments, the composition or pharmaceutical composition is for use in treating cancer.

Typically, one of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can be effective at an amount of from about 0.01 μg/kg to about 100 mg/kg per day based on total body weight. The active ingredient can be administered at once, or can be divided into a number of smaller doses to be administered at predetermined intervals of time. The suitable dosage unit for each administration can be, e.g., from about 1 μg to about 2000 mg, preferably from about 5 μg to about 1000 mg. The pharmacology and toxicology of many of such other anticancer compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be applicable to the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6.

It should be understood that the dosage ranges set forth above are exemplary only and are not intended to limit the scope of this invention. The therapeutically effective amount for individual compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can vary with factors including but not limited to the activity of the compound used, the stability of the compound used in the patient's body, the severity of the conditions to be alleviated, the total weight of the patient treated, the route of administration, the ease of absorption, distribution, and excretion of the compound by the body, the age and sensitivity of the patient to be treated, and the like, as will be apparent to a skilled artisan. The amount of administration can be adjusted as the various factors change over time.

In the pharmaceutical compositions, the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can be in any pharmaceutically acceptable salt form, as described above.

For oral delivery, the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can be incorporated into a formulation that includes pharmaceutically acceptable excipients or carriers such as binders, lubricants, disintegrating agents, and sweetening or flavoring agents, all known in the art. The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of a solution, suspension, syrup, chewing gum, wafer, elixir, and the like. If desired, conventional agents for modifying flavors, tastes, colors, and shapes of the special forms can also be included.

The compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can also be administered parenterally in the form of a solution or suspension, or in a lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. The parenteral formulations can be stored in any conventional containers such as vials and ampoules.

Routes of topical administration include nasal, bucal, mucosal, rectal, or vaginal applications. For topical administration, the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can be formulated into lotions, creams, ointments, gels, powders, pastes, sprays, suspensions, drops and aerosols. Thus, one or more thickening agents, humectants, and stabilizing agents can be included in the formulations. A special form of topical administration is delivery by a transdermal patch. Methods for preparing transdermal patches that can be used with the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, are disclosed, e.g., in Brown, et al., Annual Review of Medicine, 39:221-229 (1988), which is incorporated herein by reference.

Subcutaneous implantation for sustained release of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can also be a suitable route of administration. This entails surgical procedures for implanting one or more of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6. Hydrogels are generally known in the art. They are typically made by crosslinking high molecular weight biocompatible polymers into a network, which swells in water to form a gel-like material. Preferably, hydrogels are biodegradable or biosorbable. See, e.g., Phillips et al., J. Pharmaceut. Sci., 73:1718-1720 (1984).

The compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can also be conjugated, to a water soluble, non-immunogenic, non-peptidic, high molecular weight polymer to form a polymer conjugate. For example, one or more of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, is covalently linked to polyethylene glycol to form a conjugate. Typically, such a conjugate exhibits improved solubility, stability, and reduced toxicity and immunogenicity. Thus, when administered to a patient, compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL).

It is preferred that the covalent linkage between the polymer and one or more of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and/or the polymer itself is hydrolytically degradable under physiological conditions. Such conjugates can readily release the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, inside the body. Controlled release of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can also be achieved by incorporating one or more of the compounds of the present invention into microcapsules, nanocapsules, or hydrogels that are generally known in the art.

Liposomes can also be used as carriers for the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6. Liposomes are micelles made of various lipids such as cholesterol, phospholipids, fatty acids, and derivatives thereof. Various modified lipids can also be used. Liposomes can reduce toxicity of the compounds of the present invention, and can increase their stability. Methods for preparing liposomal suspensions containing active ingredients therein are generally known in the art, and, thus, can be used with the compounds of the present invention. See, e.g., U.S. Pat. No. 4,522,811; Prescott, Ed., Methods in Cell Biology, Volume XIV, Academic Press, New York, N.Y. (1976).

### 4. Therapeutic Methods

The present invention provides therapeutic methods for treating diseases and disorders that will respond to therapy with a Nampt inhibitor. Consequently, the present invention provides therapeutic methods for treating cancer, systemic or chronic inflammation, rheumatoid arthritis, diabetes, obesity, T-cell mediated autoimmune disease, ischemia, and other complications associated with these diseases and disorders. These therapeutic methods involve treating a patient (either a human or another animal) in need of such treatment, with a therapeutically effective amount of one or more of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising a therapeutically effective amount of one or more of the compounds of the present invention.

Additionally, the present invention provides the use of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising a therapeutically effective amount of one or more of the compounds of the present invention, for the manufacture of a medicament useful for human therapy.

In some of such embodiments, the therapy comprises therapy for the treatment of cancer, systemic or chronic inflammation, rheumatoid arthritis, diabetes, obesity, T-cell mediated autoimmune disease, ischemia, and other complications associated with these diseases and disorders, in a human patient.

In some of such embodiments, the therapy comprises therapy for the delaying the onset of, or reducing the symptoms of, cancer, systemic or chronic inflammation, rheumatoid arthritis, diabetes, obesity, T-cell mediated autoimmune disease, ischemia, and other complications associated with these diseases and disorders, in a human patient.

The present invention also comprises treating isolated cells with a therapeutically effective amount of one or more of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising a therapeutically effective amount of one or more of the compounds of the present invention.

As used herein, the phrase “treating . . . with . . . a compound” means either administering one or more of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more of the compounds of the present invention, directly to isolated cells or to an animal, or administering to cells or an animal another agent to cause the presence or formation of one or more of the compounds of the present invention inside the cells or the animal.

In some embodiments, the present invention provides a method of inhibiting the activity of Nampt in human cells comprising, contacting the cells with a compound of the present invention, such as, for example, a compound of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and a compounds of Tables 1A and 1B, 2, 3A and 3B, 4, 5, and 6. In some of such embodiments, the cells are with the body of a human patient.

Preferably, the methods of the present invention comprise administering to cells in vitro or to a warm-blood animal, particularly mammal, and more particularly a human, a pharmaceutical composition comprising an effective amount of one or more of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or another agent to cause the presence or formation of one or more of the compounds of the present invention inside the cells or the animal.

As would be appreciated by the skilled artisan, one or more of the compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, can be administered in one dose at one time, or can be divided into a number of smaller doses to be administered at predetermined intervals of time. The suitable dosage unit for each administration can be determined based on the effective daily amount and the pharmacokinetics of the compounds.

a. Treating Cancer:

In particular embodiments, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

In some embodiments, the patient is a human patient.

In some embodiments, the method comprises identifying a patient in need of such treatment. A patient having cancer can be identified by conventional diagnostic techniques known in the art, as well as by those methods discussed herein below.

As noted previously, Nampt catalyzes the first and rate-limiting step in the generation of NAD⁺ from NaM, and NAD⁺ is critical for the generation of cellular ATP by glycolysis, the citric acid cycle, and oxidative phosphorylation. By these mechanisms and others, reduction in cellular NAD⁺ levels by Nampt inhibition causes depletion of cellular ATP and, ultimately, cell death. Tumor cells are thought to be more sensitive to NAD⁺ and ATP loss than normal cells due to their higher energy needs and an increased reliance on glycolysis. Known as the “Warburg effect” (Warburg, O. On respiratory impairment in cancer cells. Science 124, 269-270 (1956)), a wide spectrum of cancer cells exhibit increased glycolysis relative to oxidative phosphorylation, despite the availability of oxygen. The shift from oxidative phosphorylation to a reliance on glycolysis is thought to result from mitochondrial damage and/or a hypoxic tumor microenvironment (reviewed in Hsu, P. P and Sabatini, D. M. Cancer cell metabolism: Warburg and beyond. Cell 134, 703-707 (2008)) and/or cellular reprogramming by oncogenes and/or tumor suppressors (reviewed in Levine, A. J. and Puzio-Kuter A. M. Science. 330, 1340-1344 (2010)). With regards to depleting energy levels in tumor cells, Nampt inhibitors would be analogous to inhibitors of other glycolytic enzymes, several of which are in cancer preclinical or clinical trials (reviewed in Pelicano H. et al. Glycolysis inhibition for anticancer treatment. Oncogene 25, 4633-4646 (2006)).

In addition to increased energy needs, tumor cells are more susceptible to NAD⁺ loss due to a higher turnover of NAD⁺ in response to DNA damage and genomic instability. According to this model, poly(ADP-ribose) polymerases (PARPs) consume NAD⁺ as they generate poly(ADP-ribose) to repair DNA in response to alkylating agents, ionizing radiation, and oxidative stress (reviewed in Galli M. et al. The nicotinamide phosphoribosyltransferase: a molecular link between metabolism, inflammation, and cancer. Cancer Res. 70, 8-11 (2010)). Indeed, an inability to replenish this NAD⁺ loss, either by reducing Nampt expression or inhibiting Nampt activity, sensitizes cells to PARP activation (Rongvaux, et al. Nicotinamide phosphoribosyl transferase/pre-B cell colony-enhancing factor/visfatin is required for lymphocyte development and cellular resistance to genotoxic stress. J. Immunol. 181, 4685-4695 (2008)).

The increased metabolic demands of cancer cells (Luo et al., Cell. 136(5):823-37 (2009). Erratum in: Cell., 2009 Aug. 21; 138(4):807.)) suggests that they should require NAD⁺ in sufficient levels to maintain cellular pools of ATP. This requirement, and the critical role played by Nampt in NAD⁺ synthesis further suggests that cancer cells have a critical need for adequate Nampt activity. Consistent with this hypothesis are reports of Nampt over-expression in colon cancers (Hufton et al., FEBS Lett. 463(1-2):77-82 (1999), Van Beijnum et al., Int. J. Cancer. 101(2):118-27 (2002)), ovarian cancers (Shackelford et al., Int J. Clin. Exp. Pathol. 3(5): 522-527 (2010)), prostate cancers (Wang et al., Oncogene 30: 907-921 (2011)) and GBM cancers (Reddy et al., Cancer Biol. Ther. 7(5):663-8 (2008)), and suggestions of the amplification of the gene encoding Nampt in multiple other cancers. Immunohistochemistry analyses suggest strong expression of Nampt occurs in greater than 20% of biopsies of: breast, lung, malignant lymphoma, ovarian, pancreatic, prostate and testicular cancers (www.proteinatlas.org). In addition to the role played by NAD⁺ as a cofactor in redox reactions, NAD⁺ also serves as a substrate for mono and poly-ADP ribosyltransferases (PARPs), class III histone deacetylases (sirtuins) and ADP-ribose cyclases. PARPs appear to be major consumers of cellular NAD⁺ (Paine et al., Biochem. J. 202(2):551-3 (1982)), and evidence exists for increased polyADP-ribosylation activity in oral cancer (Das, B. R., Cancer Lett. 73(1):29-34 (1993)), hepatocellular carcinoma (Shiobara et al., J. Gastroenterol. Hepatol. 16(3):338-44 (2001), Nomura et al., J Gastroenterol. Hepatol. 15(5):529-35 (2000)), rectal cancer (Yalcintepe et al., Braz. J. Med. Biol. Res. 38(3):361-5 (2005); Epub 2005, Mar. 8.), and leukemia and ovarian cancers (Singh N, Cancer Lett. 58(1-2):131-5 (1991)). Increased ADP-ribosylation in cancer can reflect PARPs' role in DNA repair (Durkacz et al., Nature. 283(5747):593-6 (1980); deMurcia et al., Proc. Natl. Acad. Sci. U.S.A. 94(14):7303-7 (1997), Simbulan-Rosenthal et al., Proc. Natl. Acad. Sci. U.S.A. 96(23):13191-6 (1999)) and the need to maintain genome integrity in the face of genomic instability and the resulting accumulation of point mutations, deletions, chromosomal rearrangement and aneuploidy (Hartwell and Kastan, Science. 266(5192):1821-8 (1994)). PARP-1 itself is reported to be over-expressed in breast cancer, where its expression inversely correlates with genomic instability (Biechi et al., Clin. Cancer Res. 2(7):1163-7 (1996)).

Furthermore, the Nampt transcript is known to be upregulated in colon cancers (van Beijnum J R, et al. Target validation for genomics using peptide-specific phage antibodies: a study of five gene products overexpressed in colorectal cancer. Int. J. Cancer. 101, 118-127 (2002); and Hufton S E, et al. A profile of differentially expressed genes in primary colorectal cancer using suppression subtractive hybridization. FEBS Lett. 463, 77-82 (1999)) and glioblastoma cancers (Reddy P S, et al. PBEF1/NAmPRTase/Visfatin: a potential malignant astrocytoma/glioblastoma serum marker with prognostic value. Cancer Biol. Ther. 7, 663-668 (2008)), and it remains possible that the Nampt gene is amplified in other cancers.

Thus, in one embodiment, the present invention provides a method of treating a cancer that overexpresses Nampt, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

In view of the above, it is believed that inhibition of Nampt activity would be effective in treating a wide range of cancers. Support for this assertion is found in the Examples section below. Specifically in the section entitled “Nampt Inhibition Proves Cytotoxic to a Wide Variety of Cancer Cell Types.” Consequently, the present invention provides methods of treating a wide range of cancers by administering therapeutically effective amounts of one or more of the compounds of the present invention. Specifically, it has been discovered that cancer cell types corresponding to colon, prostate, breast, NSCLC, sarcoma, pancreatic, SCLC, gastric, myeloma, ovarian, lymphoma, and glioma cancers are killed by compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6.

Thus, in one embodiment, the present invention provides a method of treating colon cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating prostate cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating breast cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating non-small-cell lung cancer (NSCLC), comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating sarcoma cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating pancreatic cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating SCLC cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating gastric cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating myeloma cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating ovarian cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating lymphoma cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating glioma cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

As used herein, the term “cancer” has its conventional meaning in the art. Cancer includes any condition of the animal or human body characterized by abnormal cellular proliferation. The cancers to be treated comprise a group of diseases characterized by the uncontrolled growth and spread of abnormal cells. Compounds of the present invention have been shown to be effective in a variety of standard cancer models, and are thus thought to have utility in treating a broad range of cancers. However, preferred methods of the invention involve treating cancers that have been found to respond favorably to treatment with Nampt inhibitors. Further, “treating cancer” should be understood as encompassing treating a patient who is at any one of the several stages of cancer, including diagnosed but as yet asymptomatic cancer.

Specific cancers that can be treated by the methods of the invention are those cancers that respond favorably to treatment with a Nampt inhibitor. Such cancers include, but are not limited to, Hodgkin's disease, non-Hodgkin's lymphoma, acute lymphocytic leukemia, chronic lymphocytic leukemia, acute myeloid leukemia, mantle-cell lymphoma, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

a. 1 Methods of Identifying Cancers Most Likely to be Susceptible to Treatment with Nampt Inhibitors

Importantly, NAD⁺ can be generated by several Nampt-independent pathways as well, including: (1) de novo synthesis from L-tryptophan via the kynurenine pathway; (2) from nicotinic acid (NA) via the Preiss-Handler pathway; and (3) from nicotinamide riboside or nicotinic acid riboside via nicotinamide/nicotinic acid riboside kinases (reviewed in Khan, J. A. et al., Nicotinamide adenine dinucleotide metabolism as an attractive target for drug discovery. Expert Opin. Ther. Targets. 11(5):695-705 (2007)). However, these different routes of NAD⁺ synthesis are generally tissue specific: The de novo pathway is present in liver, brain, and immune cells, the Priess-Handler pathway is primarily active in the liver, kidney, and heart, and Nrk2, of the nicotinamide riboside kinase pathway, is expressed in brain, heart, and skeletal muscle (Bogan, K. L. and Brenner, C. Nicotinic acid, nicotinamide, and nicotinamide riboside: a molecular evaluation of NAD⁺ precursor vitamins in human nutrition. Annu. Rev. Nutr. 28:115-30 (2008) and Tempel, W. et al., Nicotinamide riboside kinase structures reveal new pathways to NAD⁺. PLoS Biol. 5(10):e263 (2007)).

Of these alternative pathways of NAD⁺ synthesis, the Preiss-Handler pathway is perhaps the most important for cancer cells. The first and rate-limiting step of this pathway, the conversion of nicotinic acid (NA) to nicotinic acid mononucleotide (NAMN), is catalyzed by the enzyme Naprt 1.

While not wishing to be bound by theory it follows, therefore, that one way to stratify patients and to potentially expand the therapeutic window of the compounds of the present invention would be to identify those cancers with reduced or absent levels of Naprt1 expression. Such cancers would theoretically be less able to replace cellular NAD⁺ through this alternative pathway, while being treated with Nampt inhibitors. Hence, they should be more sensitive to treatment by the compounds of the present invention.

Accordingly, embodiments of the present invention include a method of identifying a cancer that is likely susceptible to treatement with a compound of the present invention, such as, for example, a compound of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and a compound of Tables 1A and 1B, 2, 3A and 3B, 4, 5, and 6. The method comprises obtaining a biopsy sample of said cancer, determining the expression level of enzymes in pathways for NAD biosynthesis (e.g. tryptophan, kynurenine pathway, nicotinic acid salvage pathway, nicotinamide riboside pathway), relative to a non-cancerous control tissue, wherein, if the expression level of enzymes in such pathways (e.g. Naprt1, Qprt, NRK-1) is reduced, relative to a non-cancerous control tissue, the cancer is identified as likely susceptible to treatement with a compound of the present invention.

In some of such embodiments, the methods of determining the expression level of the Naprt1 gene involve either determining levels of expression of the Naprt1-encoding transcript (i.e., Naprt1-encoding mRNA), or determining levels of expression of the Naprt1 protein itself. For these embodiments, any acceptable means of determining expression levels of either the Naprt1-encoding transcript, or the Naprt1 protein itself, can be utilized, and such acceptable means are well within the skill level of the artisan versed in determining expression levels of eukaryotic genes. Such acceptable means can include, for example, quantitative PCR (qPCR) to measure levels of Naprt1-encoding transcript, or ELISAs to measure levels of expressed Naprt1 protein. The specific methods involved in determining the expression of particular eukaryotic genes are well known in the art.

Additionally, embodiments of the present invention include a method of treating cancer, wherein cells of the cancer exhibit low levels of Naprt1 expression. Thus, in one embodiment, the present invention provides a method of treating a cancer that exhibit low levels of Naprt1 expression, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Cell lines were treated with exemplary compounds of the present invention and screened for NA rescue and Naprt1 expression by immunoblotting and quantitative RT-PCR (See NA Rescue and Naprt1 Expression Assays section below). Naprt1 expression was least in brain cancers, lung cancers, lymphoma, myeloma and osteosarcoma. Further, glioblastoma and sarcoma cell lines that are reported to be resistant to NA rescue have been found to have reduced Naprt1 expression (Watson, et al. Mol. Cell. Biol. 29(21):5872-88 (2009)).

Thus, in one embodiment, the present invention provides a method of treating brain cancer, such as glioblastoma, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating lung cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

Thus, in one embodiment, the present invention provides a method of treating osteosarcoma cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, to a patient.

### a.2 Methods of Limiting Toxicity of the Compounds of the Present Invention by Administering NA

In view of the NA rescue phenomenon described above, while those cancers with reduced or absent levels of Naprt1 expression should be more susceptible to treatment with the Nampt inhibitors of the present invention, administration of NA to patients having such cancers could prevent toxicity in other tissues associated with Nampt inhibition.

To support this concept, experiments were conducted to show that mice given NA survive doses of a Nampt inhibitor above the maximum tolerated dose (see also Beauparlant P., et al. Preclinical development of the nicotinamide phosphoribosyl transferase inhibitor prodrug GMX1777. Anticancer Drugs. 20(5):346-54 (2009) and Watson, et al. The small molecule GMX1778 is a potent inhibitor of NAD⁺ biosynthesis: strategy for enhanced therapy in nicotinic acid phosphoribosyltransferase 1-deficient tumors. Mol. Cell. Biol. 29(21):5872-88 (2009)). This phenomenon is referred to in the art as “NA rescue.”

Cell lines were treated with exemplary compounds of the present invention and screened for NA rescue and Naprt1 expression by immunoblotting and quantitative RT-PCR. Lack of NA rescue was greatest in brain cancers, lung cancers, lymphoma, myeloma, and osteosarcoma. Further, glioblastoma and sarcoma cell lines that are reported to be resistant to NA rescue have been found to have reduced Naprt1 expression (Watson, et al. Mol. Cell. Biol. 29(21):5872-88 (2009)).

Accordingly, in some embodiments, the methods of treating cancer disclosed herein further comprise administering nicotinic acid, or a compound capable of forming nicotinic acid in vivo, to the patient in addition to administering a compound of the present invention, such as, for example, a compound of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and a compound of Tables 1A and 1B, 2, 3A and 3B, 4, 5, and 6. In some of such embodiments, the compound of the present invention is able to be administered at dose that exceeds the maximum tolerated dose for that particular compound of the present invention as determined for mono-therapy.

In some of such embodiments, administering NA may include administering NA prior to administering one or more of the compounds of the present invention, co-administering NA with one or more of the compounds of the present invention, or first treating the patient with one or more of the compounds of the present invention, followed by thereafter administering NA.

b. Treating Systemic or Chronic Inflammation

Nampt expression in visceral adipose tissue has been found to correlate with the expression of proinflammatory genes, CD68 and TNFα (Chang et al.; Metabolism. 59(1):93-9 (2010)). Several studies have noted an increase in reactive oxygen species and activation of NF-kappaB in response to Nampt expression (Oita et al.; Pflugers Arch. (2009); Romacho et al.; Diabetologia. 52(11):2455-63 (2009)). Nampt serum levels were found to have been increased in patients with inflammatory bowel diseases and correlated with disease activity (Moschen et al.; Mutat. Res. (2009)). One study has even suggested a specific mechanism for Nampt in inflammation: High levels of Nampt increase cellular NAD⁺ levels leading to a post-transcriptional upregulation of TNF via the NAD-dependent deacetylase, SirT6 (Van Gool et al. Nat. Med. 15(2):206-10 (2009)). Further, inhibition of Nampt reduced levels of inflammatory cytokines IL-6 and TNF-α (Busso et al. PLoS One. 21; 3(5):e2267 (2008)). In another study, Nampt inhibition was found to prevent TNF-α and IFN-γ production in T-lymphocytes (Bruzzone et al.; PLoS One.; 4(11):e7897 (2009)).

In view of the above, it is believed that inhibition of Nampt activity would be effective in treating systemic or chronic inflammation resulting from a wide range of causes. Consequently, the present invention provides methods of treating systemic or chronic inflammation by administering therapeutically effective amounts of one or more of the compounds of the present invention.

c. Treating Rheumatoid Arthritis

Nampt levels increased in a mouse model of arthritis and treatment of these mice with a Nampt inhibitor reduced the arthritis symptoms (Busso et al. PLoS One. 21; 3(5):e2267 (2008)). Also, because Nampt inhibition can decrease the activity of poly(ADP ribose) polymerases (PARPs) through the dependence of PARPs on NAD as a substrate, Nampt inhibitors, either alone or in combination with PARP inhibitors can be efficacious in any ailment treatable by PARP inhibitors. In this regard, PARP inhibitors have shown efficacy in models of arthritis (Kroger et al. Inflammation. 20(2):203-215 (1996)).

In view of the above, it is believed that inhibition of Nampt activity would be effective in treating RA. Consequently, the present invention provides methods of treating RA by administering therapeutically effective amounts of one or more of the compounds of the present invention, either alone, or in combination with a PARP inhibitor.

d. Treating Obesity and Diabetes

Nampt, also known as visfatin, was described as an adipokine found in visceral fat that acted as an insulin mimetic (Fukuhara et al. Science 307:426-30 (2007)). This paper was eventually retracted and other groups have failed to confirm that Nampt binds the insulin receptor. Nevertheless, many subsequent papers continue to report correlations between Nampt expression and obesity and/or diabetes. In one, increased expression of Nampt and levels of circulating Nampt were seen in obese patients (Catalan et al.; Nutr. Metab. Cardiovasc. Dis. (2010)), although a different study found that the correlation was specific only to obese patients with type 2 diabetes (Laudes, et al.; Horm. Metab. Res. (2010)). Yet another study reported a correlation between BMI and body fat mass and Nampt plasma levels, but an inverse correlation with cerebrospinal fluid levels of Nampt (Hallschmid et al.; Diabetes. 58(3):637-40 (2009)). Following bariatric surgery, patients with pronounced weight loss showed decreased levels of Nampt mRNA in liver (Moschen et al.; J. Hepatol. 51(4):765-77 (2009)). Finally, a rare single nucleotide polymorphism was identified in Nampt that correlated with severe obesity (Blakemore, et al.; Obesity 17(8):1549-53 (2009)). In contrast to these reports, Nampt levels were not altered in rat models of obesity (Mercader et al.; Horm. Metab. Res. 40(7):467-72 (2008)). Further, circulating levels of Nampt correlated with HDL-cholesterol and inversely with triglycerides (Wang et al.; Pflugers Arch. 454(6):971-6 2007)), arguing against Nampt involvement in obesity. Finally Nampt has been show to be a positive regulator of insulin secretion by beta-cells (Revollo et al. Cell Metab. 6(5):363-75 (2007)). This effect seems to require the enzymatic activity of Nampt and can be mimicked in cell culture models by exogenous addition of NaMN.

Because Nampt inhibition can decrease the activity of poly(ADP ribose) polymerases (PARPs) through the dependence of PARPs on NAD as a substrate, Nampt inhibitor, either alone or in combination with PARP inhibitors can be efficacious in any ailment treatable by PARP inhibitors. In this regard, PARP inhibitors have shown efficacy in models of type I diabetes (Drel et al. Endocrinology. 2009 December; 150(12):5273-83. Epub 2009 Oct. 23).

In view of the above, and despite the contrasting results mentioned, it is believed that inhibition of Nampt activity would be effective in treating obesity and diabetes, and other complications associated with these, and other, metabolic diseases and disorders. Consequently, the present invention provides methods of treating obesity and diabetes, and other complications associated with these, and other, metabolic diseases and disorders, by administering therapeutically effective amounts of one or more of the compounds of the present invention.

e. Treating T-Cell Mediated Autoimmune Disease

Nampt expression has been shown to be upregulated in activated T-cells (Rongavaux et al.; J. Immunol. 181(7):4685-95 2008)) and Phase I clinical trials report lymphopenia in patients treated with Nampt inhibitors (reviewed in von Heideman et al.; Cancer Chemother. Pharmacol. (2009)). Additionally, in a mouse model of a T-cell autoimmune disease, experimental autoimmune encephalomyelitis (EAE), Nampt inhibition reduced the clinical disease score and demyelination in the spinal cord (Bruzzone et al.; PLoS One. 4(11):e7897 (2009)).

In view of the above, it is believed that inhibition of Nampt activity would be effective in treating T-cell mediated autoimmune disease, and other complications associated with diseases and disorders. Consequently, the present invention provides methods of treating T-cell mediated autoimmune disease, and other complications associated with these diseases and disorders, by administering therapeutically effective amounts of one or more of the compounds of the present invention.

f. Treating Ischemia

Because Nampt inhibition can decrease the activity of poly(ADP ribose) polymerases (PARPs) through the dependence of PARPs on NAD as a substrate, Nampt inhibitor, either alone or in combination with PARP inhibitors can be efficacious in any ailment treatable by PARP inhibitors. The PARP inhibitor FR247304 has been shown to attenuate neuronal damage in vitro and in vivo models of cerebral ischemia (Iwashita, et al. J. Pharmacol Exp. Ther. 310(2):425-36 (2004). Epub 2004 Apr. 9). Similarly there are suggestions that PARP inhibitors could be efficacious in clinical management of chronic hypoperfusion-induced neurodegenerative diseases including ocular ischemic syndrome (Mester et al. Neurotox. Res. 16(1):68-76 (2009) Epub 2009 Apr. 9) or ischemia reperfusion (Crawford et al. Surgery. 2010 Feb. 2. [Epub ahead of print]).

In view of the above, it is believed that inhibition of Nampt activity would be effective in treating ischemia and other complications associated with this condition. Consequently, the present invention provides methods of treating ischemia and other complications associated with this condition, by administering therapeutically effective amounts of one or more of the compounds of the present invention, either alone, or in combination with a PARP inhibitor.

### 5. Combination Therapy

In an additional aspect, the present invention also provides methods for combination therapy for treating cancer, systemic or chronic inflammation, rheumatoid arthritis, diabetes, obesity, T-cell mediated autoimmune disease, ischemia, and other complications associated with these diseases and disorders, by treating a patient in need thereof, with a therapeutically effective amount of one of the compounds of the present invention together with a therapeutically effective amount of one or more other compounds that have been shown to be effective in the treatment of cancer, systemic or chronic inflammation, rheumatoid arthritis, diabetes, obesity, T-cell mediated autoimmune disease, ischemia, and other complications associated with these diseases and disorders.

In some embodiments, the present invention provides methods for combination therapy for treating cancer by treating a patient (either a human or another animal) in need of the treatment with one of the compounds of the present invention together with one or more other anti-cancer therapies. Such other anti-cancer therapies include traditional chemotherapy agents, targeted agents, radiation therapy, surgery, hormone therapy, immune adjuvants, etc. In the combination therapy, one of the compounds of the present invention, such as, for example, a compound of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and a compound of Tables 1A and 1B, 2, 3A and 3B, 4, 5, and 6, can be administered separately from, or together with the one or more other anti-cancer therapies.

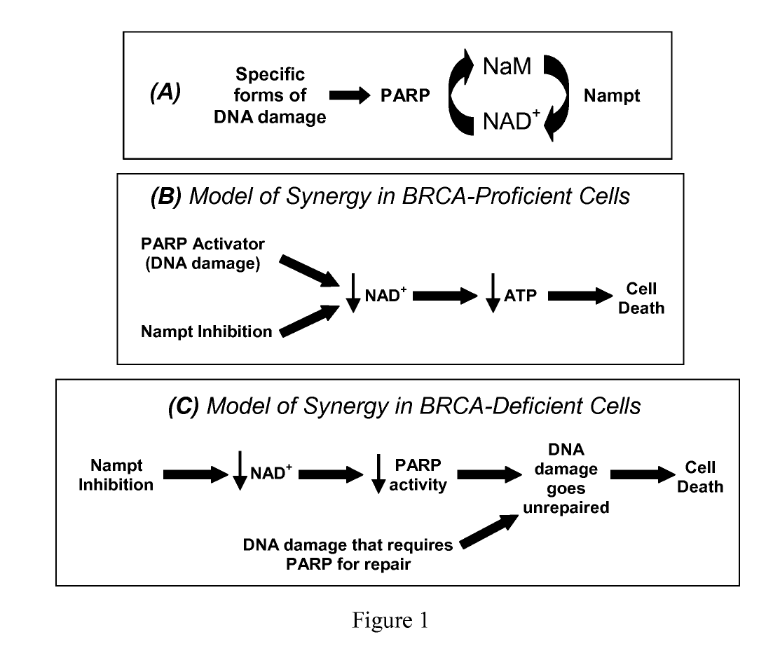

Specifically, Nampt inhibition has been shown to sensitize cells to the effects of various chemotherapeutic or cytotoxic agents. Specifically, Nampt inhibition has been shown to sensitize cells to amiloride, mitomycin C, N-methyl-N′-nitro-N-nitrosoguanidine (MNNG), melphalan, daunorubicin, cytarabine (Ara-C), and etoposide (Ekelund, S. et al. Chemotherapy 48:196-204 (2002); Rongvaux, A. et al. The Journal of Immunology 181(7):4685-95 (2008); Martinsson, P. et al. British Journal of Pharmacology 137:568-73 (2002); Pogrebniak, A. et al. European Journal of Medical Research 11(8):313-21 (2006)). It is also thought that lactate dehydrogenase A inhibitors, prostaglandin H2 synthase 2 (PGHS-2) inhibitors, combined with Nampt inhibitors would be effective cancer treatments. Although the mechanism(s) behind this synergy between Nampt inhibitors and other cell killing agents has not been fully explored, Nampt inhibition causes a drop in cellular levels of NAD⁺ at doses and times of exposure that are not overtly toxic to the cell. Without wishing to be bound by theory, it is believed that sub-lethal NAD⁺ drops render cells vulnerable to other cytotoxic agents, and particularly to compounds which activate the DNA repair enzyme poly(ADP-ribose) polymerase (PARP), since PARP requires NAD⁺ as a substrate and consumes NAD⁺ during its enzymatic action (FIG. 1A).

Accordingly, in some embodiments, the present invention provides the methods of treating cancer disclosed herein further comprise administering a therapeutically-effective amount of a PARP activator to the patient in addition to administering a compound of the present invention, such as, for example, a compound of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and a compound of Tables 1A and 1B, 2, 3A and 3B, 4, 5, and 6.

Additionally, in some of such embodiments, the cells of the cancer have functional homologous recombination (HR) systems. Also, in some of such embodiments, the methods further comprise identifying the cells of the cancer as having functional HR systems. Methods of performing such identification are known in the art. Furthermore, in addition to a PARP activator, in some embodiments, the methods of treating cancer disclosed herein further comprise administering a therapeutically effective amount of a non-DNA damaging agent to the patient, wherein the non-DNA damaging agent is not a PARP activator and not a compound of the present invention. For example, where the cancer has functional HR systems for repairing DNA damage, then an additional chemotherapeutic could be administered that does not rely on DNA damage for efficacy. Chemotherapeutics the do not damage DNA are known in the art.

Agents or treatments that may be capable of activating the PARP enzyme include but are not limited to: alkylating agents (methyl methane sulfonate (MMS), N-methyl-N′nitro-N-nitrosoguanidine (MNNG), Nitrosoureas (N-methyl-N-nitrosourea (MNU), streptozotocin, carmustine, lomustine), Nitrogen mustards (melphalan, cyclophosphamide, uramustine, ifosfamide, clorambucil, mechlorethamine), alkyl sulfonates (busulfan), platins (cisplatin, oxaliplatin, carboplatin, nedaplatin, satraplatin, triplatin tetranitrate), non-classical DNA alkylating agents (temozolomide, dacarbazine, mitozolamide, procarbazine, altretamine)), radiation (X-rays, gamma rays, charged particles, UV, systemic or targeted radioisotope therapy), and other DNA damaging agents such as: topoisomerase inhibitors (camptothecin, beta-lapachone, irinotecan, etoposide), anthracyclines (doxorubicin, daunorubicin, epirubicin, idarubicin, valrubicin, mitoxantrone), reactive oxygen generators (menadione, peroxynitrite), and anti-metabolites (5-FU, raltetrexed, pemetrexed, pralatrexate, methotrexate, gemcitabine, thioguanine, fludarabine, azathioprine, cytosine arabinoside, mercaptopurine, pentostatin, cladribine, folic acid, floxuridine).

It is further believed that tumors or tumor cell lines treated with compounds that directly or indirectly inhibit the enzyme thymidylate synthase (TS) can also be more susceptible to Nampt inhibitors, such as compounds of the present invention.

Accordingly, in some embodiments, the present invention provides the methods of treating cancer disclosed herein further comprise administering a therapeutically-effective amount of a thymidylate synthase inhibitor to the patient in addition to administering a compound of the present invention, such as, for example, a compound of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and a compound of Tables 1A and 1B, 2, 3A and 3B, 4, 5, and 6.

In some embodiments, the thymidylate synthase inhibitor directly or indirectly inhibits thymidylate synthase. Thymidylate synthase inhibitors include 5-FU, raltitrexed, pemetrexed, and other TS inhibitors developed over the past decades.

It is further believed that agents that promote aberrant uracil incorporation into DNA can also make subjects being administered such agents more susceptible to Nampt inhibitors, such as compounds of the present invention. Any inhibitor of thymidylate synthase (TS) would cause uracil incorporation into DNA. Other agents, such as inhibitors of dihydrofolate reductase (e.g. methotrexate) have also been shown to cause uracil to aberrantly incorporate into DNA.

Accordingly, in some embodiments, the present invention provides the methods of treating cancer disclosed herein further comprise administering a therapeutically-effective amount of agents that promote aberrant uracil incorporation into DNA, to the patient in addition to administering a compound of the present invention, such as, for example, a compound of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and a compound of Tables 1A and 1B, 2, 3A and 3B, 4, 5, and 6.

In view of the above, some embodiments of the present invention comprises the use of the compounds of the present invention with a second chemotherapeutic agent that has been discovered to work synergistically with one or more of the compounds of the present invention, such as compounds or treatments that activate PARP, induce DNA damage, inhibit TS, and/or promote aberrant uracil incorporation into DNA, or inhibit proteasomes or specific kinases.

In certain embodiments of this aspect of the invention, the second chemotherapeutic agent is selected from, at least, methyl methanesulfonate (MMS), mechlorethamine, streptozotocin, 5-fluorouracil (5-FU), raltitrexed, methotrexate, bortezomib, PI-103, and dasatinib.

In HCT116 cells, the potent and selective PARP inhibitor olaparib failed to synergize with Nampt inhibitors—in fact antagonism was observed, in which olaparib protected cells somewhat from Nampt inhibitor-induced death. PARP inhibitors are relatively benign to cells (like HCT116 cells) that have a functional homologous recombination (HR) system to repair double stranded DNA damage (Ashworth A. Journal of Clinical Oncology 26(22):3785-90 (2008)). In fact, the model (FIG. 1A) predicts that inhibiting an enzyme, such as PARP, that consumes NAD⁺ would protect HR-proficient cells from Nampt inhibition. However, in cells that have lost the function of BRCA tumor suppressors, HR function is compromised, and these cells are killed by PARP inhibitors (Ashworth A. (2008) Journal of Clinical Oncology 26(22):3785-90). Thus, it was hypothesized that PARP inhibitors, while being antagonistic with Nampt inhibitors in most cells, would be synergistic in cells with BRCA mutations that render the cells HR-deficient (FIG. 1B). Indeed, in MDA-MB-436 cells, which have a loss of BRCA1 function, Nampt inhibitors (including compounds of the present invention) and the PARP inhibitor olaparib synergized in causing cell death. This result is particularly encouraging as it suggests that the drug combination of one of the compounds of the present invention plus a PARP inhibitor would be antagonistic in normal cells (FIG. 1A), but synergistic in cells that do not have functional HR systems, such as cells that have lost BRCA tumor suppressor function (FIG. 1B).

Other routes of HR deficiency in oncogenesis (other than BRCA sequence mutation) could also lead to sensitivity to PARP inhibition plus Nampt inhibitor combination therapy. These additional mutations, which lead to a “BRCAness” phenotype, include, as documented in ovarian cancers, BRCA1 promoter methylation and upregulation of BRCA inhibitors, such as the protein EMSY (Bast R. C. and Mills G. B. Journal of Clinical Oncology 28(22):3545-8 (2010)). Further studies have demonstrated that mutation of the tumor suppressor gene phosphatase and tensin homolog (PTEN), a gene frequently mutated in a variety of cancers, reduces HR function and sensitizes cells to PARP inhibitors (Mendes-Pereira A. M. et al. EMBO Molecular Medicine 1:315-322 (2009)). Providing more evidence for the BRCAness model of PARP inhibitor sensitivity, in a cell biological study using RNA interference, mutation of any of 12 different genes functionally important for HR sensitized cells to PARP inhibitors (McCabe et al. Cancer Research 66(16): 8109-15 (2006)). Finally, a recent paper has demonstrated that cells in hypoxic conditions, such as those found in the center of virtually all solid tumors, are selectively killed by PARP inhibitors (Chan et al. Cancer Research 70(2): 8045-54 (2010)).

Accordingly, in some embodiments, the present invention provides the methods of treating cancer disclosed herein further comprise administering a therapeutically-effective amount of a PARP inhibitor to the patient in addition to administering a compound of the present invention, such as, for example, a compound of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and a compound of Tables 1A and 1B, 2, 3A and 3B, 4, 5, and 6.

In some of such embodiments, the cells of the cancer do not have functional homologous recombination (HR) systems. In some of such embodiments, the methods of treating cancer further comprise identifying the cells of the cancer as not having functional HR systems. Methods of performing such identification are known in the art.

In some of such embodiments, the PARP inhibitor is olaparib, AG014699/PF-01367338, INO-1001, ABT-888, Iniparib, BSI-410, CEP-9722, MK4827, or E7016.

In some of such embodiments, the methods further comprise administering a therapeutically effective amount of a DNA damaging agent to the patient, wherein the DNA damaging agent is other than a PARP inhibitor. DNA damaging agents are known in the art and include topoisomerase inhibitors (camptothecin, beta-lapachone, irinotecan, etoposide), anthracyclines (doxorubicin, daunorubicin, epirubicin, idarubicin, valrubicin, mitoxantrone), reactive oxygen generators (menadione, peroxynitrite), and anti-metabolites (5-FU, raltetrexed, pemetrexed, pralatrexate, methotrexate, gemcitabine, thioguanine, fludarabine, azathioprine, cytosine arabinoside, mercaptopurine, pentostatin, cladribine, folic acid, floxuridine).

Studies were expanded to investigate synergistic combinations of Nampt inhibitors and standards of care in particular cancer types. Cancer cell lines used in these studies represented cancer types found to be sensitive to Nampt inhibition [e.g. non-Hodgkins lymphoma, multiple myeloma, glioma, non-small cell lung carcinoma (NSCLC), small cell lung carcinoma (SCLC), ovarian cancer and colorectal cancer]. Standards of care in these cancer types tested in synergy experiments included: 4-HC (the pre-activated form of cyclophosphamide), doxorubicin, vincristine, prednisolone, dexamethasone, melphalan, thalidomide, bortezomib, temozolomide, cisplatin, paclitaxel, gefitinib, 5-FU, oxaliplatin, irinotecan, and etoposide. Synergistic cytotoxicity was found when compounds of the present invention were combined with 4HC in small-cell lung cancer (SCLC) and glioma, temozolomide in glioma, and 5-FU in colon cancer.

Another specific example of an active agent with which the compounds of the present invention can be co-administered is the immune adjuvant L-1-methyl tryptophan (L-1MT). In studies of co-administration of L-1MT with another inhibitor of Nampt (i.e., AP0866 [also known as FK866 or WK175]), the combination was shown to provide an additive inhibitory effect on tumor growth of murine gastric and bladder tumors in immune-competent mice (Yang et al. Exp. Biol. Med. 235:869-76 (2010)).

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of temozolomide, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of 4HC, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of 5-FU, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of L-1MT, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of methyl methanesulfonate (MMS), to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of mechlorethamine, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of streptozotocin, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of raltitrexed, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of methotrexate, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of bortezomib, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb10, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of PI-103, to a patient.

Thus, in one embodiment, the present invention provides a method of treating cancer, comprising administering a therapeutically effective amount of one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, or a pharmaceutical composition comprising one or more compounds of the present invention, such as, for example, the compounds of Formulae I, Ia, Ia1, Ia2, Ib, Ib1, Ib2, Ib3, Ic, Id, II, IIa, IIa1, IIa2, IIa3, IIa4, IIb, IIb1, IIb2, IIb3, IIb4, IIb5, IIb6, IIb7, IIc, IIc1, IId, IId1, III, IIIa, IIIa1, IIIa2, IIIa3, IIIa4, IIIa5, IIIa6, IIIb, IIIb1, IIIb2, IIIb3, IIIb4, IIIb5, IIIb6, IIIb7, IIIb8, IIIb9, IIIb1, IIIb11, IIIc, IV, IVa, IVa1, IVa2, Iva3, IVa4, IVa5, IVa6, IVb, IVb1, IVb2, IVb3, IVb4, IVb5, IVb6, IVb7, IVb8, and IVc, as illustrated herein, and the compounds of Tables 1A and 1B, 2, 3A and 3B, 4, 5, and 6, and administering a therapeutically-effective amount of dasatinib, to a patient.

In the case of combination therapy, a therapeutically effective amount of one or more other therapeutically effective compounds can be administered in a separate pharmaceutical composition, or alternatively included in the same pharmaceutical composition of the present invention which contains one of the compounds of the present invention. One or more of the compounds of the present invention can be administered together in the same formulation with the one or more other compounds that have been shown to be effective in the treatment of cancer, systemic or chronic inflammation, rheumatoid arthritis, diabetes, obesity, T-cell mediated autoimmune disease, ischemia, and other complications associated with these diseases and disorders, in the same formulation or dosage form. Thus, the present invention also provides pharmaceutical compositions or medicaments for combination therapy, comprising an effective amount of one or more of the compounds of the present invention, and an effective amount of at least one other compound that has been shown to be effective in the treatment of cancer, systemic or chronic inflammation, rheumatoid arthritis, diabetes, obesity, T-cell mediated autoimmune disease, ischemia, and other complications associated with these diseases and disorders.

The compounds of the present invention can also be administered in combination with another active agent that synergistically treats or prevents the same symptoms or is effective for another disease or symptom in the patient being treated, so long as the other active agent does not interfere with, or adversely affect, the effects of the compounds of the present invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, immune adjuvants, and the like.

### 6. Methods of Making the Compounds of the Present Invention

In an additional aspect, the present invention provides methods of the making the compounds of the present invention. Embodiments of methods of making the compounds of the present invention, and intermediates used in their synthesis, are provided in the General Synthetic Schemes and Specific Syntheses Procedures below. In all cases, the syntheses were begun using commercially-available starting materials.

In some embodiments, a method of making a compound, comprises reacting

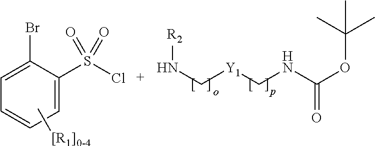

under suitable conditions to yield the intermediate

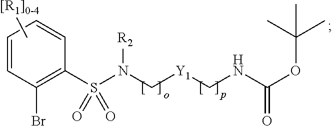

converting said intermediate to a second intermediate

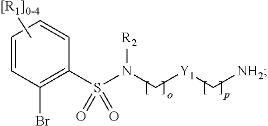

reacting said second intermediate with Y—(CH₂)_{q}—NH₂ to yield

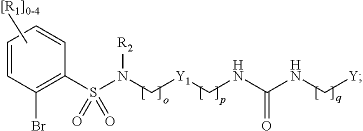

wherein Y, Y₁, o, p, and q, are as defined for Formula III and wherein R₁, and R₂ are as defined for Formulae IIIa4 or IIIb5.

In some embodiments, a method of making a compound, comprises reacting

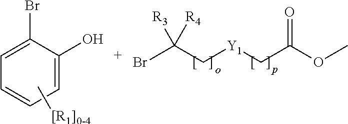

under suitable conditions to yield the intermediate

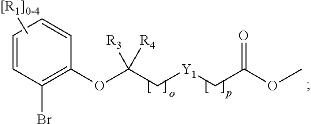

converting said intermediate to a second intermediate

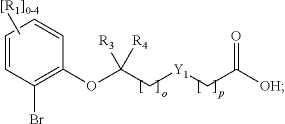

reacting said second intermediate with Y—(CH₂)_{q}—NH₂ to yield

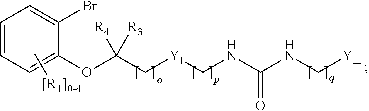

wherein Y, Y₁, o, p, and q, are as defined for Formula III, and wherein R₁, R₃, and R₄ are as defined for Formula IIIa3 or IIIb4.

### Synthetic Schemes

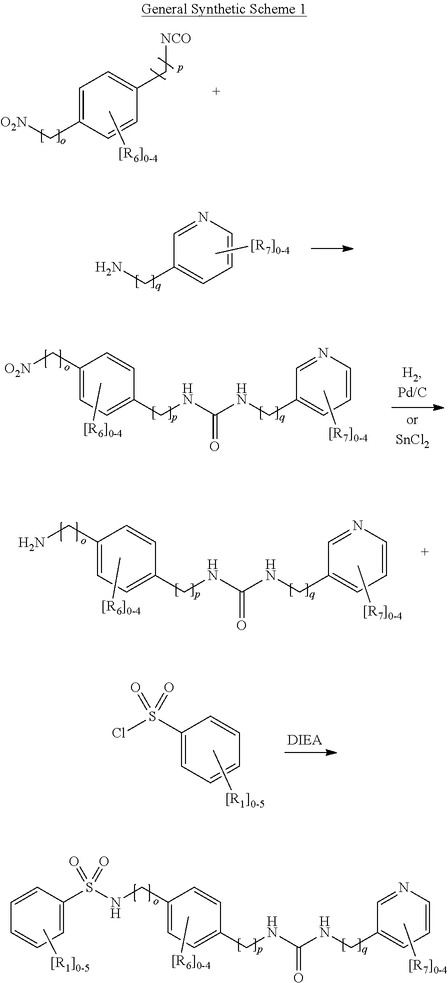

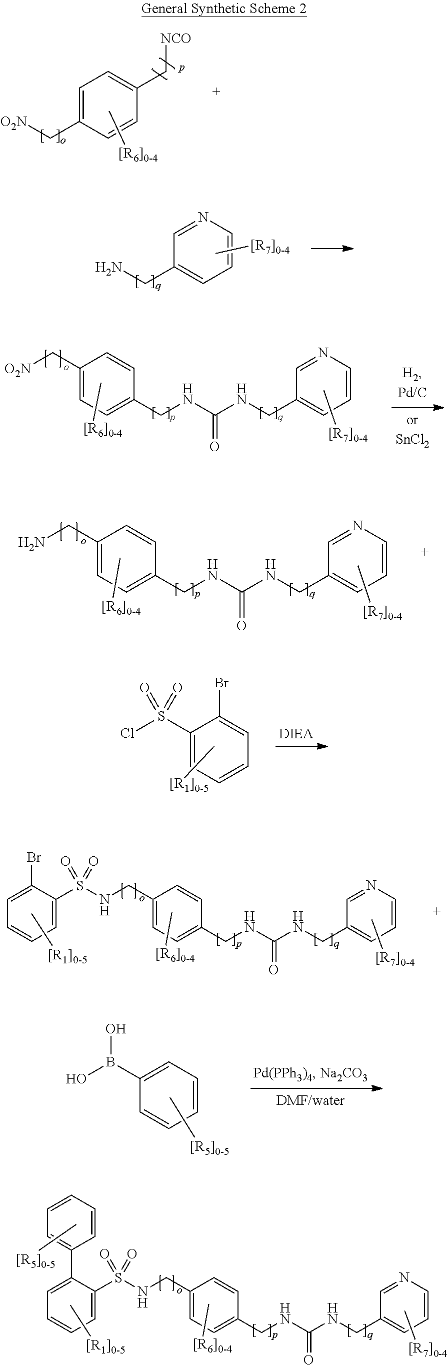

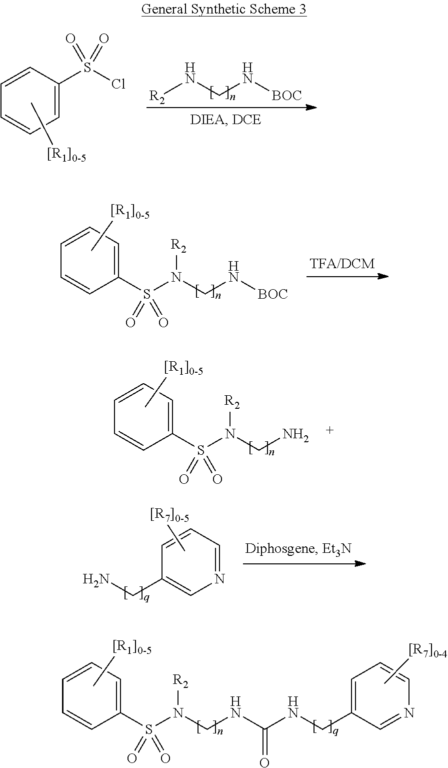

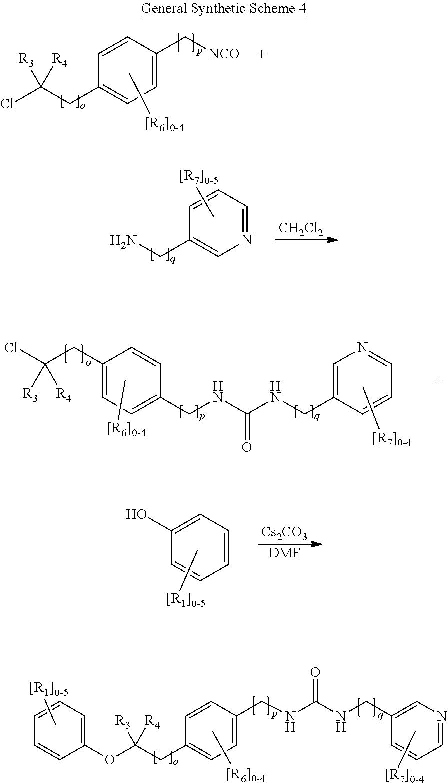

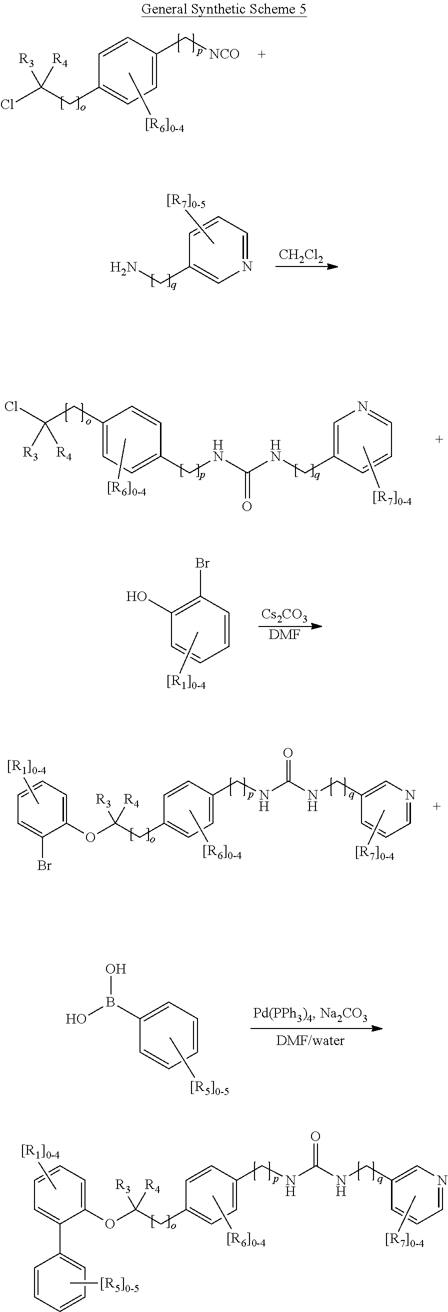

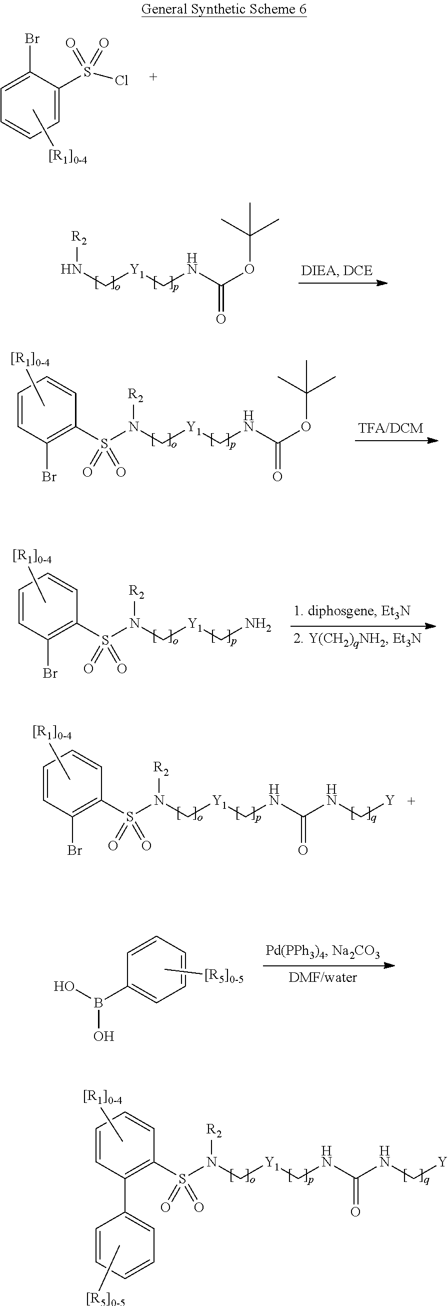

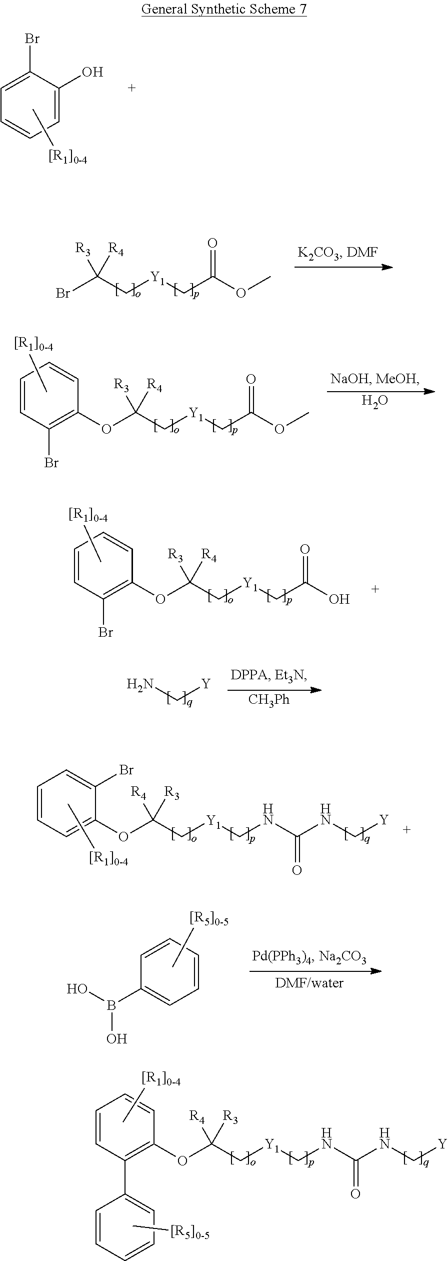

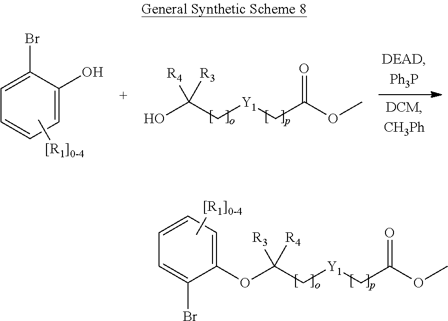

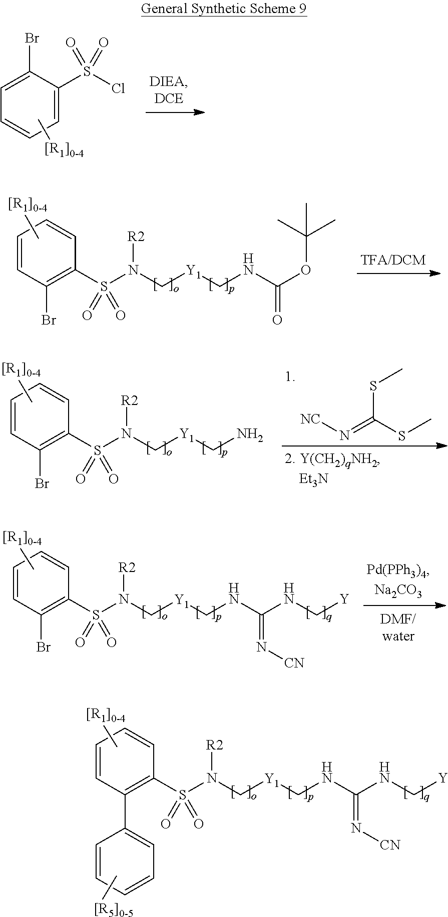

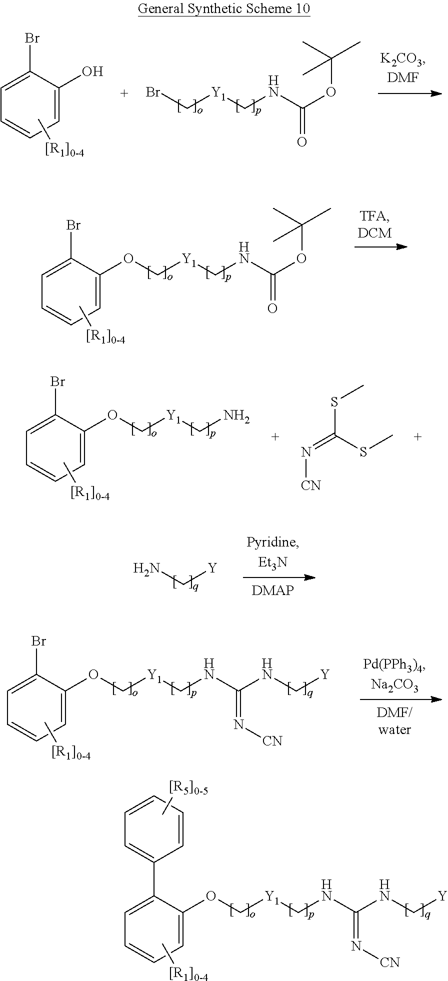

### Specific Syntheses:

Procedure 1

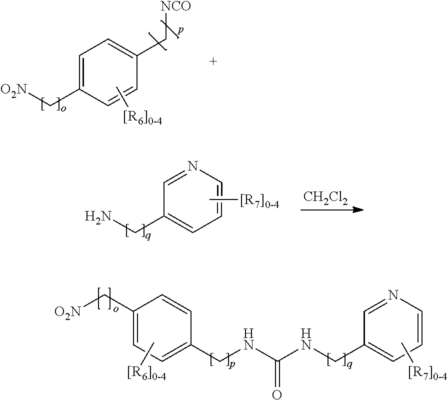

The appropriate amine (1.0 eq.) was added to a solution of the appropriate isocyanate (1.0 eq.) in CH₂Cl₂ dropwise at room temperature. The product was collected by filtration and dried under vacuum.

Procedure 2

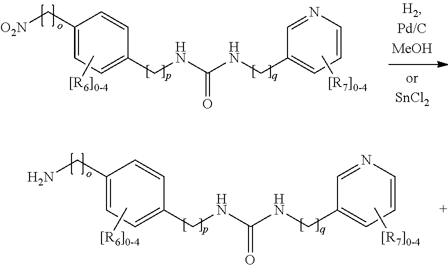

Procedure for R₆═H.

Pd/C (10%) was added to a mixture of the appropriate aryl nitro compound in methanol (ca. 0.2 M). The reaction mixture was evacuated and back filled with H₂ (3×), and was stirred under H₂ (balloon) overnight. The mixture was filtered through celite, and the filtrate was concentrated to give the desired product.

Procedure for Some of R₆=Halogen.

SnCl₂ (3-6 eq.) was added to a solution of the appropriate ary nitro compound in EtOH or EtOAc and stirred at reflux for 4 hrs to overnight. The solvent (if EtOH was used) was removed, and the resulting residue was dissolved in EtOAc and washed with saturated NaHCO₃. The aqueous layer was extracted (2×), and the combined organic extracts were washed with brine, dried (Na₂SO₄), filtered and concentrated. The resulting residue was purified by Si-gel chromatography to give the desired product.

Procedure 3

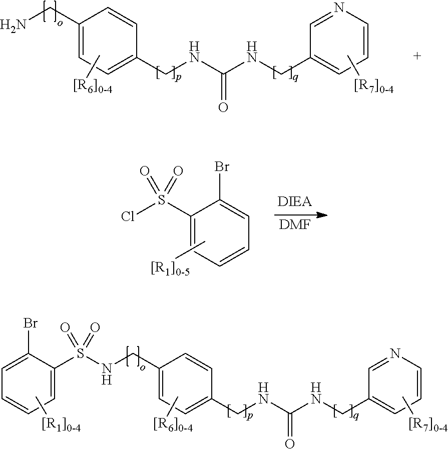

The appropriate sulfonyl chloride (1.1 eq.) was added to a solution of DIEA (DIEA=Hiinig's base, 1.5 eq.) and the appropriate amine (1.0 eq.), in DMF (ca. 0.2 M). The mixture was stirred overnight at room temperature. The solvent was removed and the resulting residue was washed with water. The material was suspended in MeOH/EtOAc, and the product was collected by filtration and dried under vacuum. When necessary, the product was purified by silica gel chromatography.

Procedure 4

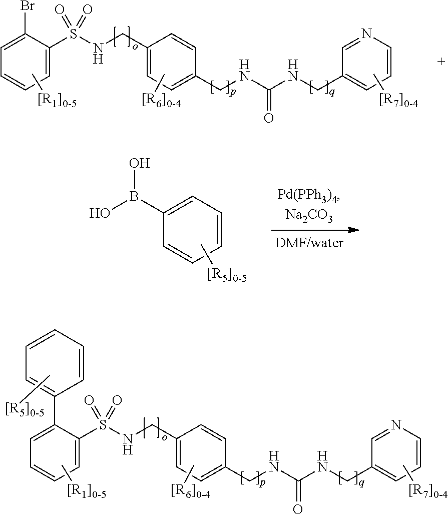

A mixture of the appropriate aryl bromide (1.0 eq.), the appropriate boronic acid (1.5 eq.), and Na₂CO₃ (2.8 eq.) in DMF/water (10:1, 0.2M) was flushed with N₂. Pd(PPh₃)₄ (0.07 eq.) was added, the mixture was flushed with N₂, and stirred overnight at 110° C. The reaction mixture was cooled to room temperature and the insoluble material was removed by filtration. The filtrate was concentrated and the resulting material was purified by silica gel chromatography.

Procedure 5

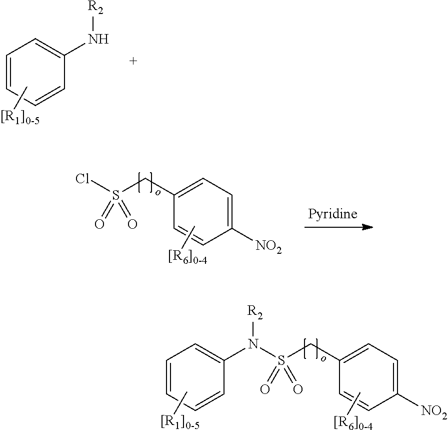

A mixture of the appropriate amine and the appropriate sulfonyl chloride were stirred in pyridine (ca. 0.2 M) overnight at room temperature. The pyridine was removed, and the residue was dissolved in EtOAc and washed with 1N HCl. The organic layer was washed with brine, dried (Na₂SO₄), filtered and concentrated. If needed, the product was purified by silica gel chromatography.

Procedure 6

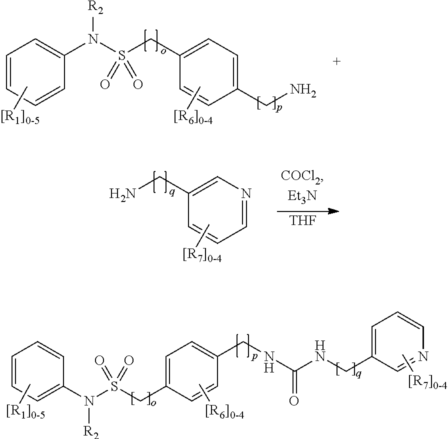

A solution of the appropriate amine (1.0 eq.) and Et₃N (3.2 eq) in THF was added to a solution of phosgene (COCl₂—20% in toluene) in THF (Ca. 0.2 M) drop wise at 0° C. The mixture was warmed to room temperature and stirred 1-2 hours. The reaction mixture was flushed with N₂ and the solvent was removed under vacuum at low temperature to remove excess COCl₂. The residue was dissolved in THF (0.2 M), the second appropriate amine was added, and the resulting mixture was stirred overnight at room temperature. The mixture was concentrated and purified by silica gel chromatography.

Procedure 7

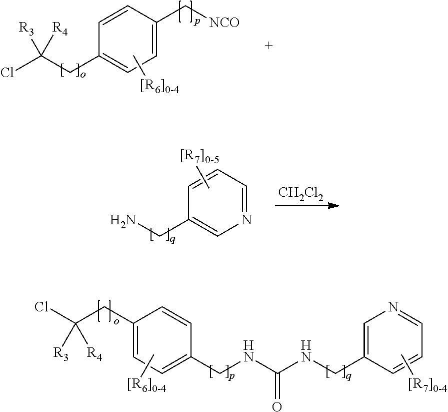

The appropriate aminopyridine (1.0 eq.) was added dropwise to a solution of the appropriate chloroisocyanate (1.0 eq.) in CH₂Cl₂ (ca. 0.2 M) at 0° C. The resulting mixture was stirred at 0° C. for 45 minutes. The solid product was collected by filtration and dried under vacuum.

Procedure 8

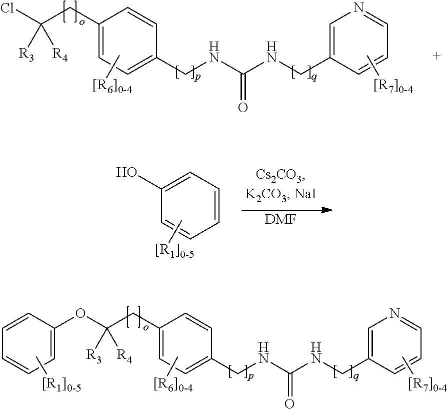

A mixture of the appropriate phenol (1.1 eq.), and Cs₂CO₃ (1.5 eq.) in DMF (ca. 0.2 M) was stirred for 45 min at room temperature. The appropriate chloride (1.0 eq.) was added, and the reaction mixture was stirred at 80° C. overnight. The mixture was cooled to room temperature. The insoluble material was removed by filtration, and the filtrate was concentrated. The resulting residue was purified by silica gel chromatography.

Procedure 9

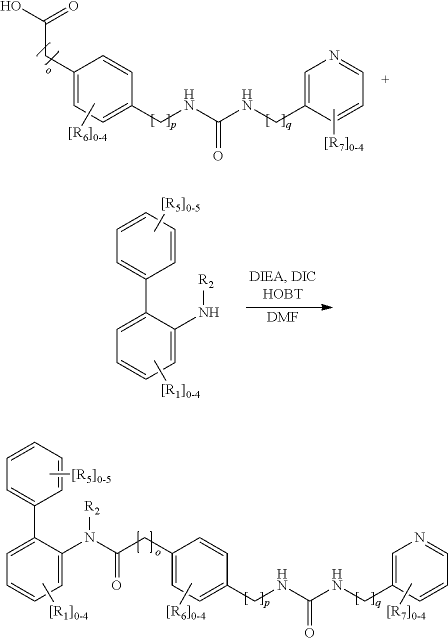

DIEA (3 eq.) was added to a mixture of the appropriate amine, the appropriate benzoic acid, DIC (1.2 eq.) and Hydroxyvenzotriazole (HOBt) (1.2 eq.) in DMF The mixture was stirred at room temperature overnight. The solution was concentrated and purified by reverse phase (RP)-HPLC.

Procedure 10

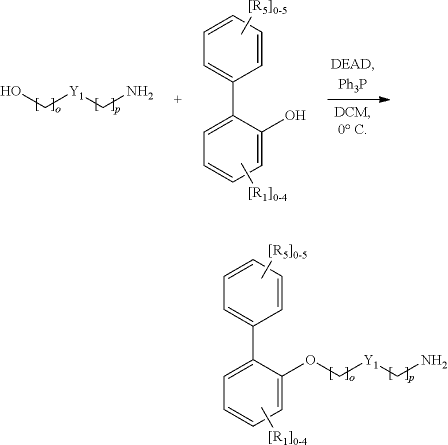

DEAD (1.2 eq., 2M in PhCH₃) was added at to a mixture of the appropriate phenol, the appropriate amino alcohol, and PPh₃ (1.2 eq.) at 0° C. in DCM or THF. The solution was warmed to room temperature and stirred overnight, concentrated and purified by silica gel chromatography.

Alternatively, the appropriate N-boc-amino alcohol can be used in the above procedure, followed by TFA/DCM deprotection as follows: TFA (−3 mL/mmol) was added to the N-boc-amine in DCM and the solution stirred at room temperature for 30 min. The solution was concentrated and dissolved in EtOAc, washed with saturated NaHCO₃, dried with Na₂SO₄, concentrated and, if needed, purified by silica gel chromatography.

Procedure 11

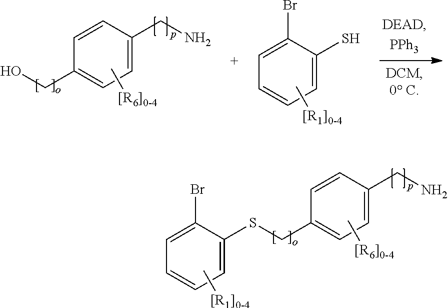

DEAD (1.2 eq., 2M in PhCH₃) was added at 0° C. to the appropriate thiol, the appropriate alcohol, and PPh₃ (1.2 eq.) in DCM. The solution was stirred at room temperature overnight, concentrated and purified by silica gel chromatography.

Procedure 12

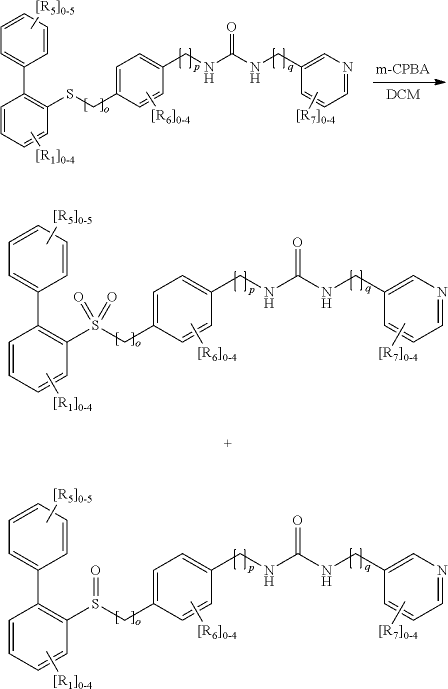

m-CPBA (2.2 eq.) was added to the appropriate sulfide in DCM and the mixture was stirred at room temperature for two hours. The resulting mixture of sulfoxide and sulfone was concentrated and purified by RP-HPLC.

Procedure 13

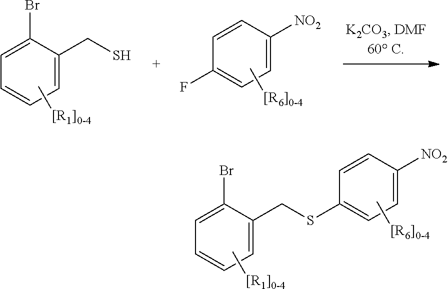

Fluoro-1-nitrobenzene, the appropriate thiol, and K₂CO₃ (3 eq.) were heated at 60° C. in DMF for 64 hours. The solution was diluted with EtOAc, washed with 10% HCl, dried with Na₂SO₄ and concentrated to give the desired product.

Procedure 14

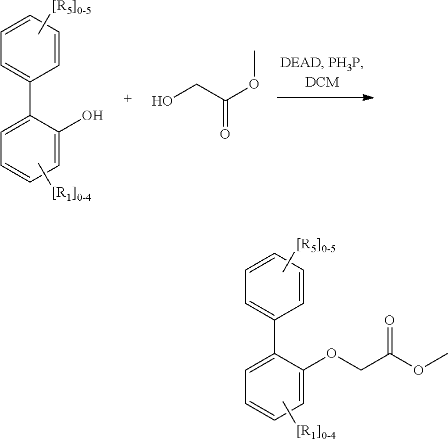

DEAD (1.2 eq., 2M in PhCH₃) was added at to a mixture of the appropriate phenol, the appropriate methyl glycolate, and PPh₃ (1.2 eq.) at 0° C. in DCM. The solution was stirred at room temperature overnight, concentrated and purified by silica gel chromatography.

Procedure 15

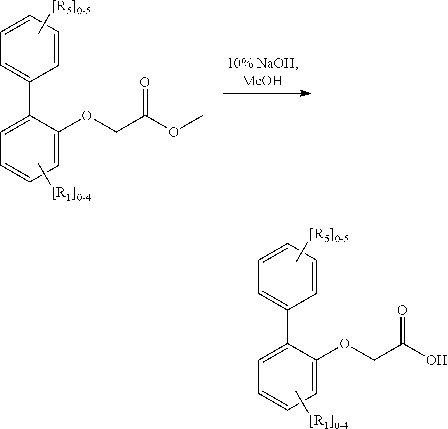

The appropriate ester was dissolved in methanol followed by the addition of NaOH (10%, 2.5 eq). The reaction mixture was stirred at room temperature for 4 hours acidified and extracted with ethyl acetate. After concentration, the acid was used without further purification.

Procedure 16

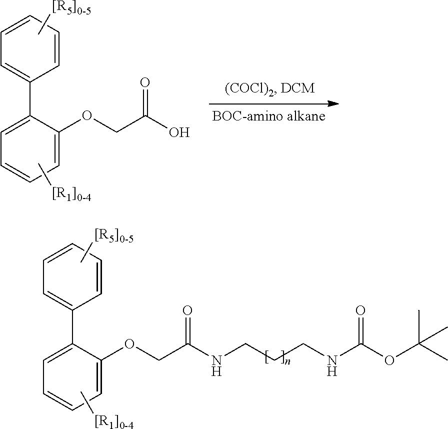

The appropriate carboxylic acid was dissolved in DCM and oxalyl chloride was added. After stirring 30 minutes at room temperature, the mixture was concentrated and the resulting acid chloride was used as is for subsequent reactions.

The appropriate mono BOC protected diamine (1 eq.) was added to a solution of the crude acid chloride (1 eq.) from above in DCM and Et₃N (3 eq.). After stirring the mixture overnight at room temperature, the mixture was washed with HCl (1N) and the organic layer was concentrated and used without further purification.

Procedure 17

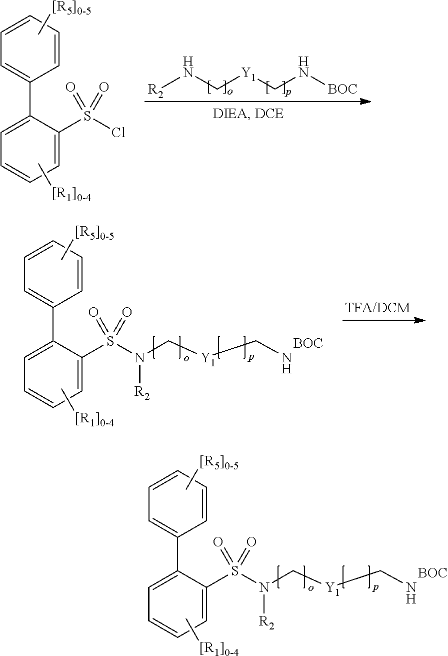

The appropriate mono-N-boc-diamine (1.2 eq.) was added to the appropriate sulfonyl chloride, DIEA (1.5 eq.) in DCE and the solution stirred at room temperature for 90 minutes. 10% HCl and DCM was added and the organic layer was dried with Na₂SO₄ or using a phase separator column and concentrated. TFA and DCM were added and the solution stirred at room temperature for 30-60 minutes and concentrated.

Procedure 18

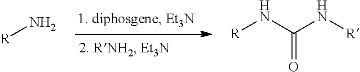

Diphosgene (0.6 eq.) and Et₃N (1.2 eq.) were added to the appropriate amine in DCM at 0° C. and the solution stirred at 0° C. for 20-120 minutes. Et₃N (3 eq.) and the second appropriate amine (1.2 eq.) were added at 0° C. and the solution was warmed to room temperature overnight. The solution was concentrated and purified by silica gel chromatography or RP-HPLC.

Procedure 19

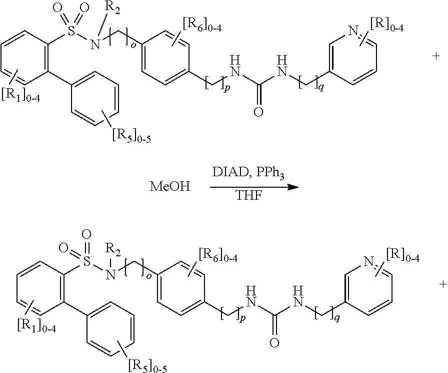

(diisopropyl azodicarboxylate) (2.0 eq.) was added to a mixture of the appropriate sulfonamide (1.0 eq.), methanol (2.0 eq.), and PPh₃ (2.0 eq.) in THF (0.2 M) dropwise at 0° C. After addition, the mixture was warmed to room temperature and stirred overnight. The solvent was removed and the resulting solution was concentrated and purified by silica gel chromatography.

Procedure 20

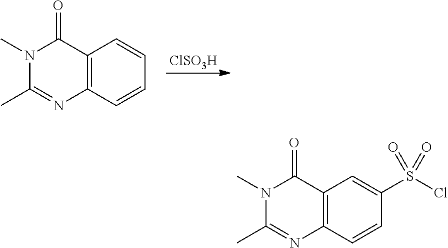

Chlorosulfonic acid (4.10 mL, 62.6 mmol) was slowly added to 2,3-dimethylquinazolin-4(3H)-one (1.09 g, 0.26 mmo). The resulting mixture was gradually heated to 140° C. and stirred for 3 hours at the same temperature. After cooling to room temperature, the viscous reaction mixture was poured into crushed ice. The precipitate was collected by filtration, washed with H₂O, and dried under vacuum to afford the desired compound.

Procedure 21

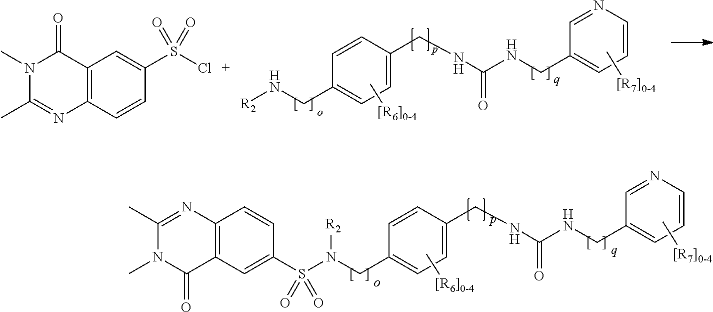

To a solution of the appropriate amine (0.495 mmol) in DMF (1 mL) was added successively pyridine (2.06 mmol), 2,3-dimethyl-4-oxo-3,4-dihydroquinazoline-6-sulfonyl chloride (0.495 mmol), and DMAP (0.041 mmol) at 0° C. After the mixture had been stirred for 10 hours at room temperature, the precipitate was removed by filtration and washed with MeOH. The combined filtrates were concentrated in vacuum and purified by preparatory HPLC to afford the title compound as a TFA salt.

Procedure 22

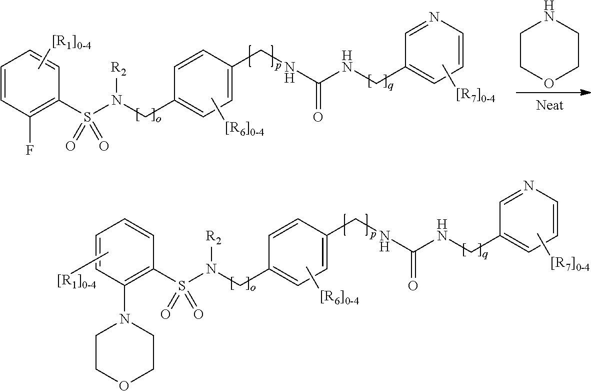

A mixture of the appropriate fluorophenyl sulfonamide (0.13 mmol) and the appropriate amine (0.50 mL) in a vial was heated at 100° C. with stirring overnight. The mixture was concentrated under reduced pressure and then more fluorophenyl sulfonamide (0.50 mL) was added and again heated at 100° C. with stirring overnight. The mixture was concentrated under reduced pressure and purified by using HPLC to afford the desired product.

Procedure 23

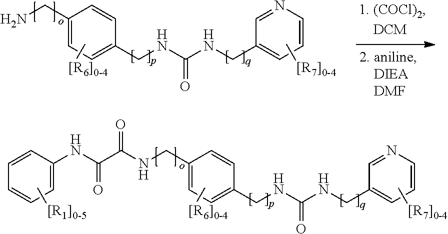

Oxalyl chloride (1.2 eq.) was added to an appropriate amine in DCM (0.2 M) and the solution stirred at room temperature for 15 minutes. The second appropriate amine (1.5 eq.) and Et₃N (2 eq.) were added in DMF (1 mL) and the solution was stirred at ambient temperature overnight. The mixture was concentrated and purified by RP-HPLC.

Procedure 24

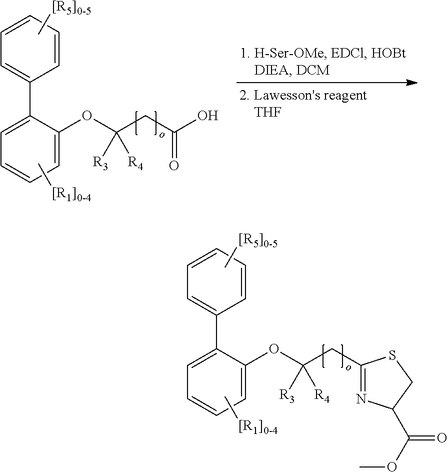

DIEA (3 eq.) was added to the appropriate carboxylic acid, H-Ser-OMe, EDCI (1.2 eq.) and HOBt (1.2 eq.) in DCM (0.2 M) and the solution stirred at room temperature overnight. The solution was washed with 10% (aq) HCl, saturated NaHCO₃, dried with Na₂SO₄, concentrated and purified by silica gel chromatography (0-60% EtOAc/hex). To the resulting oil was added THF (0.2 M) and Lawesson's reagent (1.2 eq.) and then the solution was heated at reflux overnight, concentrated, and purified by silica gel chromatography (0-60% EtOAc/hex).

Procedure 25

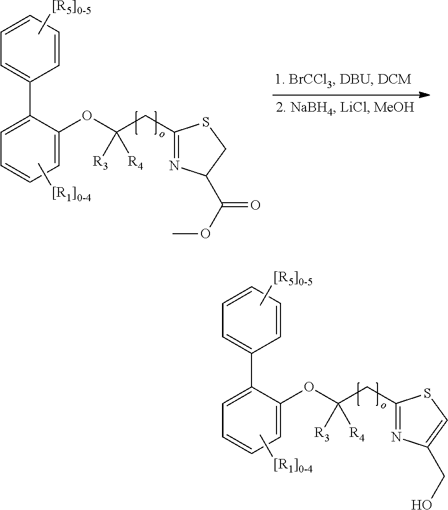

BrCCl₃ (1.1 eq.) was added to the appropriate ester and DBU (1.1 eq.) in DCM (0.15 M) and the solution stirred at room temperature for 90 minutes. The solution was diluted with more DCM, washed with 10% HCl, dried with Na₂SO₄ and concentrated. To the resulting material was added LiCl (1.2 eq.) and MeOH (0.2 M). NaBH₄ (1.2 eq.) was added and the solution was stirred at room temperature overnight. Another portion of LiCl/NaBH₄ (1.2 eq. each) was added and the solution was stirred overnight. The mixture was diluted with EtOAc, washed with 10% (aq) HCl, dried with Na₂SO₄, and concentrated. The resulting material is purified by silica gel chromatography (0-100% EtOAc/hex).

Procedure 26

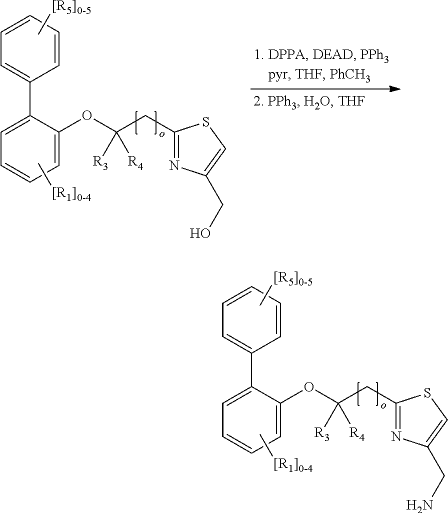

DEAD (2M in PhCH₃, 1.2 eq.) was added slowly to Diphenylphosphoryl azide (DPPA) (1.2 eq.), PPh₃ (1.2 eq.) and pyridine (1.2 eq.) in THF (0.2 M) at 0° C. The solution was stirred at 0° C. for 5 minutes. The appropriate alcohol was added in a small amount of THF and the solution is allowed to warm to room temperature overnight. The solution was concentrated and purified by silica gel chromatography (0-100% EtOAc/hex). To the resulting oil was added PPh₃ (1.2 eq.) and THF (0.2 M) and then the solution was stirred for 30 minutes. Water (10% volume of THF) was added and the mixture was heated at reflux overnight, concentrated, and purified by silica gel chromatography (0-15% MeOH/DCM).

Procedure 27

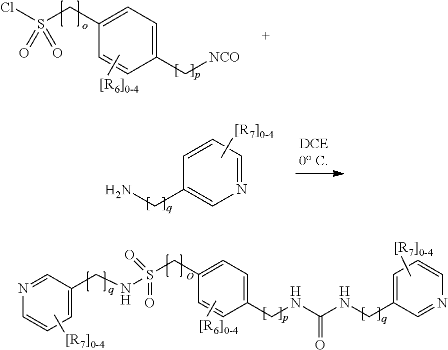

The appropriate amine (1.0 eq.) was added to a solution of the appropriate sulfonyl chloride-isocyanate (1.0 eq.) in CH₂Cl₂ dropwise at 0° C. The reaction mixture was allowed to warm to room temperature with stirring overnight. The mixture was concentrated under reduced pressure and purified using RP-HPLC to afford the desired product.

Procedure 28

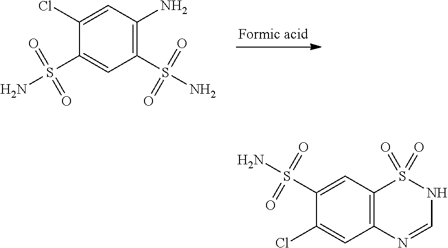 [SMILES: Nc1cc(Cl)c(S(N)(=O)=O)cc1S(N)(=O)=O ; NS(=O)(=O)c1cc2c(cc1Cl)N=CNS2(=O)=O]

To a round bottomed flask 4-amino-6-chloro-benzene-1,3-disulfonamide (11.4 g, 39.89 mmol) was added to stirring in formic acid (150 mL). The reaction mixture was heated at 125° C. with stirring (48 hrs). The solution was cooled, water was added until a white precipitate formed. The precipitate was collected via filtration, dried and carried on without further purification to yield the desired product.

Procedure 29

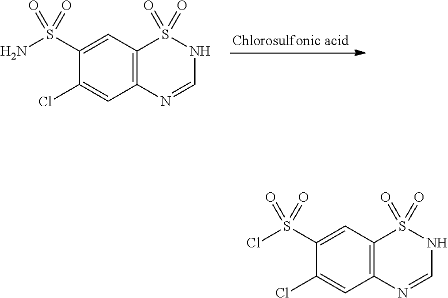 [SMILES: NS(=O)(=O)c1cc2c(cc1Cl)N=CNS2(=O)=O ; O=S(=O)(Cl)c1cc2c(cc1Cl)N=CNS2(=O)=O]

To a round bottomed flask 6-chloro-1,1-dioxo-2H-benzo[e][1,2,4]thiadiazine-7-sulfonamide (7.4 g, 25.02 mmol) was added. To this was added chlorosulfonic acid (37.5 mL) slowly. Upon complete addition the reaction mixture was heated to 100° C. for 2 hours. The mixture was allowed to cool to room temperature then cautiously and slowly poured over ice. The desired product was isolated via filtration as a white solid.

Procedure 30

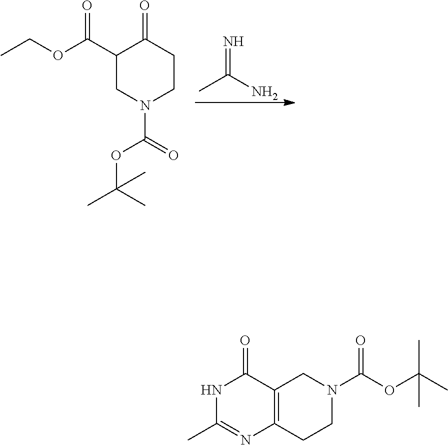

To a round bottomed flask 1-tert-butyl-3-ethyl-4-oxopiperidine-1,3-dicarboxylate (3.8 g, 14.01 mmol) was added with acetamidine HCl (1.46 g, 15.41 mmol, 1.1 eq.) stirring in EtOH (50 mL). While stirring, solid sodium metal (0.71 g, 29.42 mmol, 2.1 eq.) was added. Upon dissolution, the reaction mixture was heated at 100° C. over the weekend. The reaction mixture was allowed to cool and filtered to remove solids. The EtOH solution was then concentrated to yield the desired product as a cream colored solid.

Procedure 31

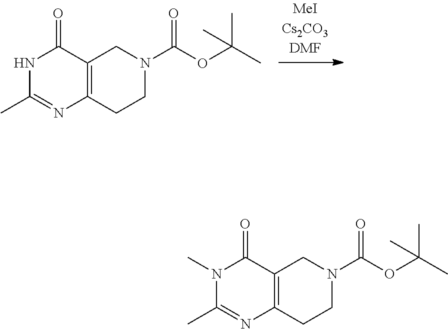 [SMILES: Cc1nc2c(c(=O)[nH]1)CN(C(=O)OC(C)(C)C)CC2 ; Cc1nc2c(c(=O)n1C)CN(C(=O)OC(C)(C)C)CC2]

To a large vial tert-butyl 2-methyl-4-oxo-3,5,7,8-tetrahydropyrido[4,3-d]pyrimidine-6-carboxylate (1.5 g, 5.65 mmol) was added and dissolved in DMF (15 mL, anhyd.). Cesium carbonate (2.76 g, 8.48 mmol) and Iodomethane (0.39 mL, 6.12 mmol) were added and the mixture was stirred at room temperature (4 hours). LCMS showed the major peak to be desired product. The reaction mixture was concentrated over SiO₂ and purified via silca gel chromatography (0-20% DCM/MeOH).

Procedure 32

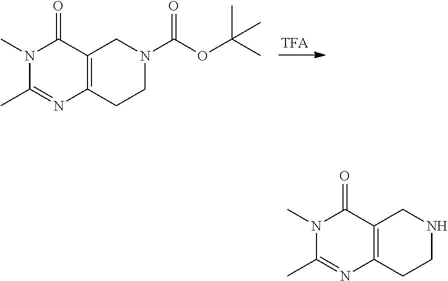

To a round bottomed flask tert-butyl 2,3-dimethyl-4-oxo-7,8-dihydro-5H-pyrido[4,3-d]pyrimidine-6-carboxylate (1.0 g, 3.58 mmol) was added stirring in DCM (10 mL) and TFA (5 mL) or HCl dioxane (4M, 10-20 eq.) at room temperature (2 hr). Concentrated to yield the desired product and carried on without purification.

Procedure 33

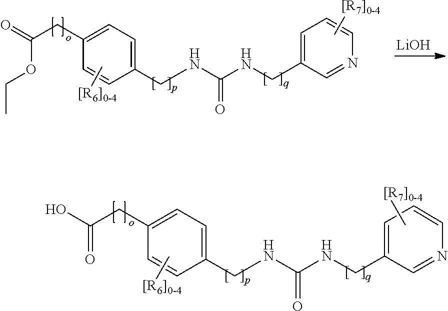

The appropriate ester (1.14 g, 3.81 mmol) was added with stirring in LiOH (1N, 10 mL) and THF (10 mL) at room temperature overnight. The mixture was concentrated to remove solvent and redisolved in 20% MeOH/DCM, filtered to remove solids. The mother liquor was concentrated to yield the desired product as a white solid.

Procedure 34

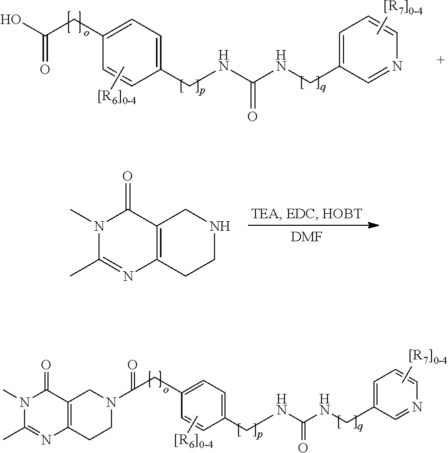

TEA (3.0 eq.) was added to a mixture of the appropriate aniline, the appropriate benzoic acid (1.1 eq.), EDC (1.5 eq.) and HOBt (1.5 eq.) in DMF The mixture was stirred at room temperature overnight. The solution was concentrated and purified by reverse phase (RP)-HPLC.

Procedure 35

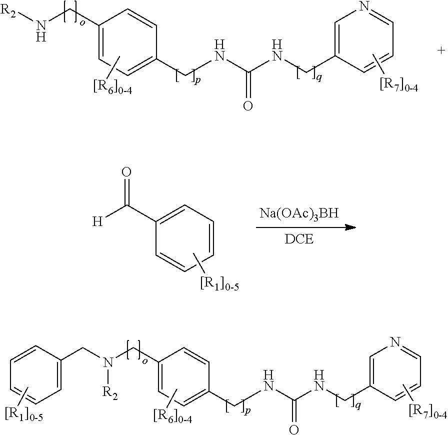

To a mixture of the appropriate aniline (1.0 eq.) and appropriate benzaldehyde (1.3 eq.) in DCE (0.2 M) was added Na(OAc)₃BH (1.5 eq.), followed by AcOH (2-4 drops), The resulting mixture was stirred overnight at room temperature. The reaction was quenched with the addition of 10% NaOH (amount equal to solvent volume), the layers were separated, and the organic layer was concentrated and purified by reverse phase chromatography.

Procedure 36

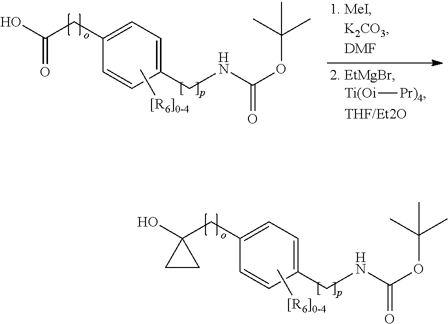

Iodomethane (1.2 eq.) was added to the appropriate carboxylic acid and K₂CO₃ (3 eq.) in DMF (0.5 M). The mixture was stirred at room temperature overnight. Ethyl acetate was added, the solution washed with 10% (aq) HCl, water, and brine, dried with Na₂SO₄ and concentrated. The resulting solid was dissolved in THF (0.2 M). Ti(OPrⁱ)₄ (1.05 eq.) was added followed by EtMgBr (3.0 M in Et₂O, 5 eq.). The resulting solution was stirred at room temperature overnight. Saturated NH₄Cl was added, the solution was filtered over celite, and the filtered solid was washed with DCM. The filtrate layers were separated and the organic layer was dried with Na₂SO₄, concentrated, and purified by gradient silica gel chromatography (0-30% EtOAc/hex).

Procedure 37

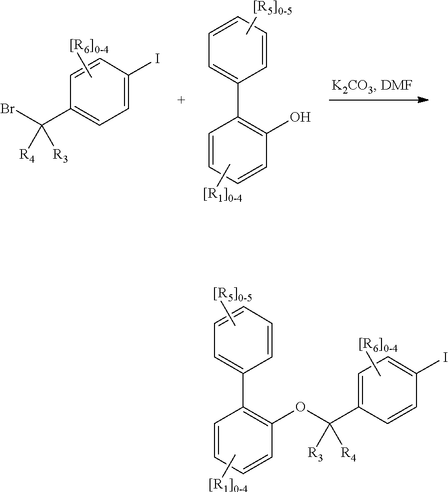

To a large vial, an appropriate benzyl bromide was dissolved in DMF (1.0M). To this was added the appropriate alcohol (1.0 eq.), and K₂CO₃ (2.0 eq.). The reaction was heated overnight at 60° C. Crude reaction mixture was concentrated over SiO₂ and purified via gradient silica gel chromatography 0-20% EtOAc/Hex.

Procedure 40

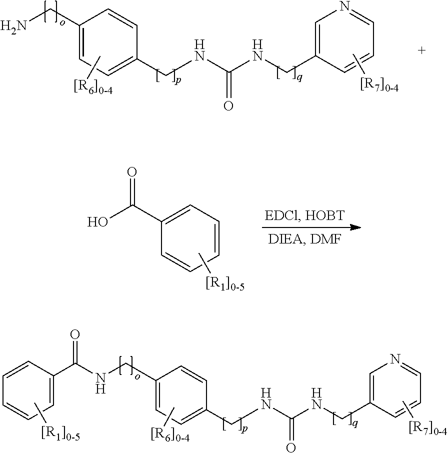

A mixture of the appropriate amine (1.0 eq.), appropriate benzoic acid (1.2 eq.), 1-Ethyl-3-(3-dimethylaminopropyl)carbodiimide (EDCI) (1.3 eq.), HOBT (1.3 eq.) and DIEA (4.0 eq.) in DMF (0.2 M) was stirred overnight at room temperature. The reaction mixture was concentrated and purified by reverse phase chromatography.

Procedure 41

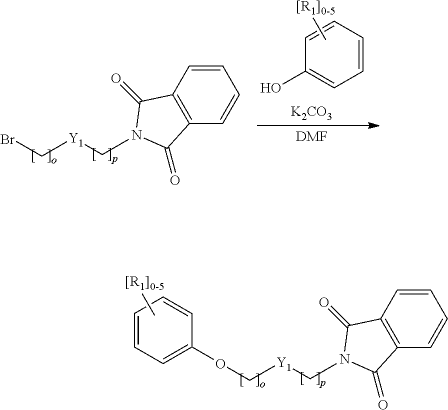

To a solution of the desired alcohol (1.2 eq.) in DMF was added K₂CO₃ (3.0 eq.), followed by the desired thalimide protected amino alcohol (1.0 eq.). The reaction was heated to 80° C. for 24 hours. Water was added and the precipitate was filtered to give the desired product, which was dried under vacuum.

Procedure 42

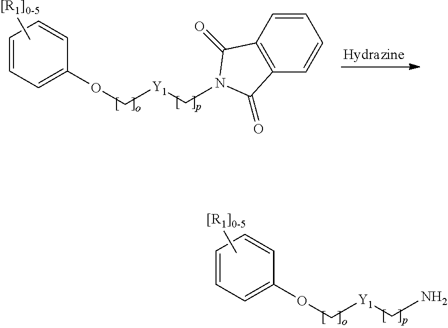

To a thalimide protected amine (9.0 g) was added anhydrous hydrazine (20 ml). This mixture was allowed to stir at room temperature for 18 hours. Acetonitrile was added and the resulting solid was filtered. The mother liquor was concentrated. An aqueous workup was performed. The organic layer was dried over Na₂S₂O₄, filtered, and concentrated under vacuum to give the desired product.

Procedure 43

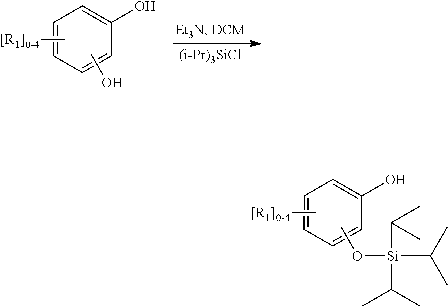

Triisopropylsilyl chloride (TIPSC1) (1.2 eq.) was added to the appropriate dialcohol (1 eq.) and Et₃N (1.5 eq.) in DCM. The solution was stirred at room temperature for 2 h., washed with 10% HCl, dried with Na₂SO₄, concentrated and purified by silica gel chromatography to give the desired product.

Procedure 44

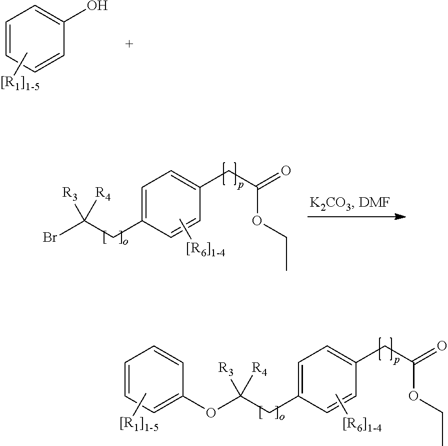

DMF (1 mL/mmol) is added to the desired alcohol (1 eq.) and the appropriate bromide (1 eq.). K₂CO₃ (3 eq.) was added and the solution heated at 60° C. for 3 h. The solution was cooled, diluted with EtOAc (˜5× volume of DMF), and washed with 10% HCl, water, and brine (3-5× volume of DMF each). The organic layer was dried with Na₂SO₄, filtered, and concentrated.

Procedure 45

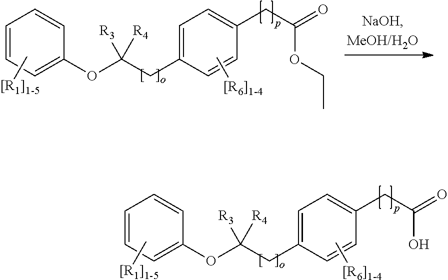

MeOH or EtOH (1 mL/mmol) was added to a substituted ester. NaOH (10% w/w aqueous, 1 mL/mmol, ˜2.5 eq.) was added and the solution heated at reflux for 1 h. Workup A: The solution was cooled, diluted with EtOAc (˜5× volume of MeOH), and washed with 10% HCl. The organic layer was dried with Na₂SO₄, filtered, and concentrated. The resulting solid is triturated with EtOAc to remove residual phenol.

Workup B: The solution was cooled and the solvent was removed under vacuum. The resulting residue was dissolved in water and acidified to ˜pH 2. The precipitate was collected by filtration and dried under vacuum.

Procedure 46

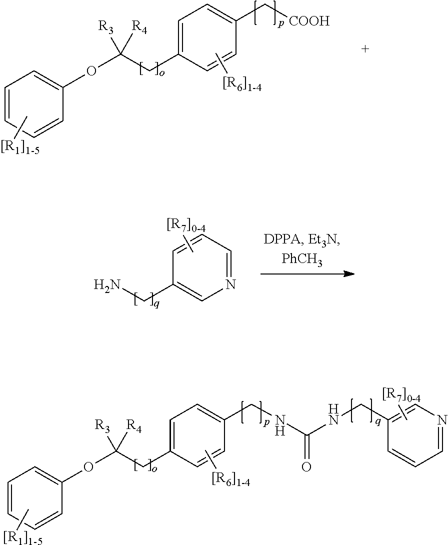

Diphenylphosphoryl azide (DPPA) (1 eq.) was added to a substituted carboxylic acid and Et₃N (1 eq.) in toluene (0.2 M), and the solution was heated at reflux for 2 h. The reaction mixture was cooled to room temperature, the appropriate amine (1.2 eq.) was added, and the solution was stirred at rt. for 2-3 h. The solution was concentrated over silica gel and purified by silica gel chromatography (0-15% MeOH/DCM). The resulting yellow oil was taken up in a minimum of DCM, added to a large excess of hexanes, stirred for 0.5-2 h., and the product was filtered.

Procedure 47

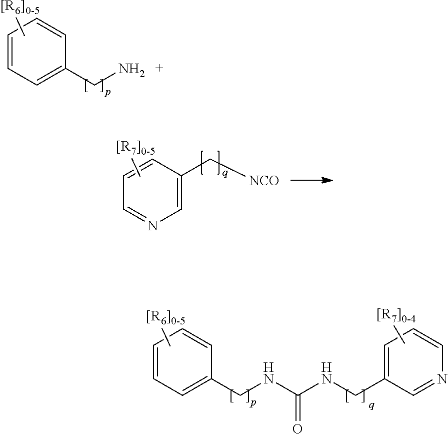

To a solution of the appropriate isocyanate (1 eq.) in 2-methyltetrahydrofuran was added the appropriate amine (1.2 eq.). The mixture was heated to 65° C. for 18 hours. The mixture was concentrated and purified by reverse phase HPLC.

Procedure 48

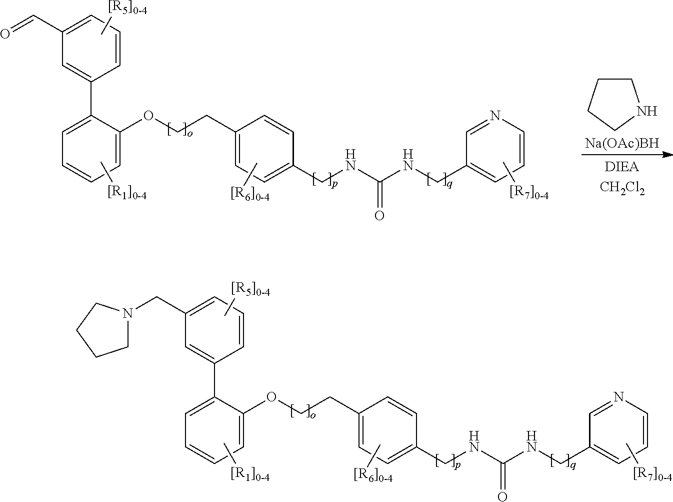

To the appropriate aldehyde (0.12 mmol) in dichloroethane (2 mL) was added the desired amine (0.23 mmol) and diisopropylethylamine (0.23 mmol). After stirring for 5 minutes sodium triacetoxyborohydride (0.23 mmol) was added to the mixture. Upon completion of the reaction as determined by LCMS, the reaction was quenched with addition of MeOH (5 mL). The reaction was concentrated and purified via reverse phase (RP)-HPLC.

Procedure 49

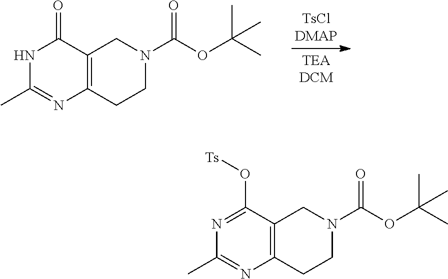 [SMILES: Cc1nc2c(c(=O)[nH]1)CN(C(=O)OC(C)(C)C)CC2 ; Cc1ccc(S(=O)(=O)Oc2nc(C)nc3c2CN(C(=O)OC(C)(C)C)CC3)cc1]

To a round bottomed flask tert-butyl 2-methyl-4-oxo-3,5,7,8-tetrahydropyrido[4,3-d]pyrimidine-6-carboxylate (2.0 g, 7.54 mmol) was dissolved in DCM, followed by the addition of TEA (1.2 eq.), and DMAP (0.1 eq.). The mixture was stirred at room temperature overnight. The mixture was poured over a prepacked silica and purified by silica gel chromatographty (0-10% DCM/MeOH). The desired product was isolated as a tacky white solid (2.73 g, 86%).

Procedure 50

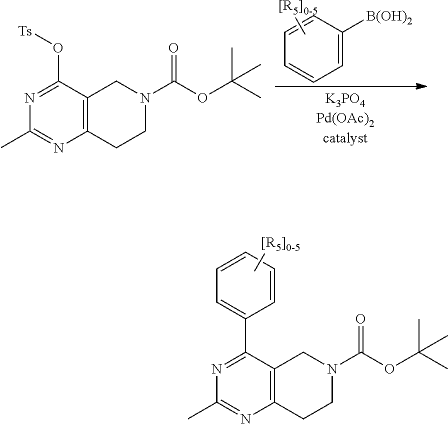

To a round bottomed flask tert-butyl 2-methyl-4-(p-tolylsulfonyloxy)-7,8-dihydro-5H-pyrido[4,3-d]pyrimidine-6-carboxylate was added (2.73 g, 6.51 mmol) along with the appropriate boronic acid (3.0 eq.), K₃PO₄ (6.0 eq.), and 2-dicyclohexylphosphino-biphenyl (0.1 eq.) followed by sparging with nitrogen (10 min). To this mixture was added dioxane (100 mL) and H₂O (1.0 mL). Again the mixture was sparged with nitrogen (5 min). Pd(OAc)₂ was added to the mixture and was once again sparged with nitrogen (5 min). The mixture was heated to 80° C. with stirring over the weekend. The reaction was cooled to room temperature, filtered to remove solids, rinsing with EtOAc. The filtrate was then transferred to a seperatory funnel containing EtOAc (250 mL) and sodium bicarbonate solution (sat, 200 mL). The aqueous layer was extracted twice with EtOAc and the combined organics were washed with brine and dried over MgSO₄. The mixture was concentrated and purified by silica gel chromatography (0-10% DCM/MeOH) to yield the desired product as a tan. (1.6 g, 75% yield).

Procedure 51

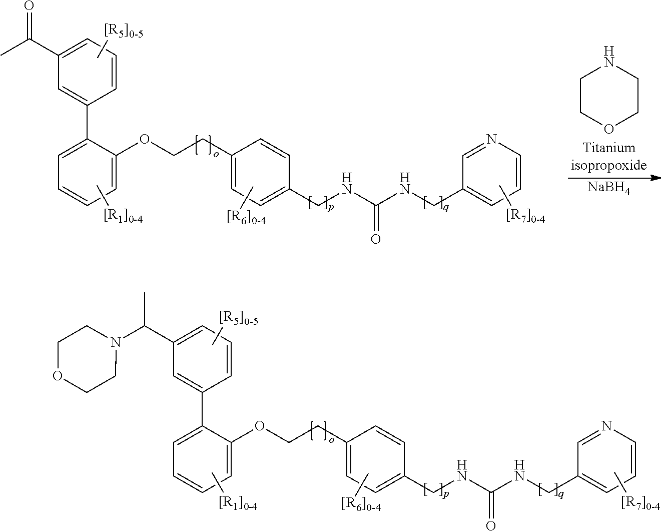

The appropriate aldehyde or ketone was dissolved in DCM. To the mixture was added titanium tetraisopropoxide (2.6 eq.) and the appropriate amine (1.5 eq.). The mixture was stirred at room temperature overnight. To the mixture was added methanol (1 vol eq. to DCM) and NaBH₄ (1.5 eq.) while stirring at room temperature until complete reduction was seen by LCMS. Two drops NaOH (2N) were added and the resulting mixture was filtered through celite and rinsed with DCM. The resultant filtrate was concentrated over SiO₂ and purified 0-20% DCM/MeOH and, if necessary, reverse phase C₁₈ HPLC.

Procedure 52

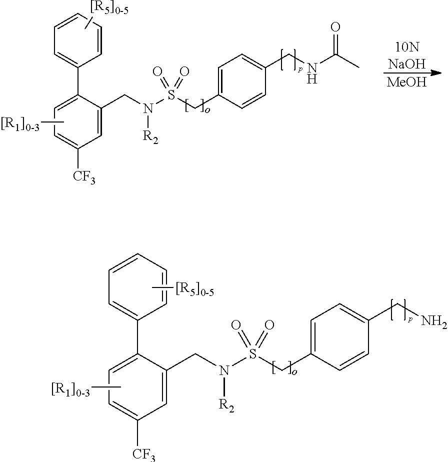

To a round bottomed flask the appropriate compound containing the N-actetate group was added in MeOH. 10N NaOH (25-50 eq.) was added to the mixture and heated to reflux. The reaction was monitored by LCMS until complete deprotection occurred. Upon completion, the reaction was cooled and neutralized with HCl and the solution was transferred to a separatory funnel and extracted with DCM (3×). The combined organics were dried over MgSO₄ and concentrated over SiO₂. The crude mixture was purified via silica gel chromatography 0-20% DCM/MeOH to yield the desired deprotected amine.

Procedure 53

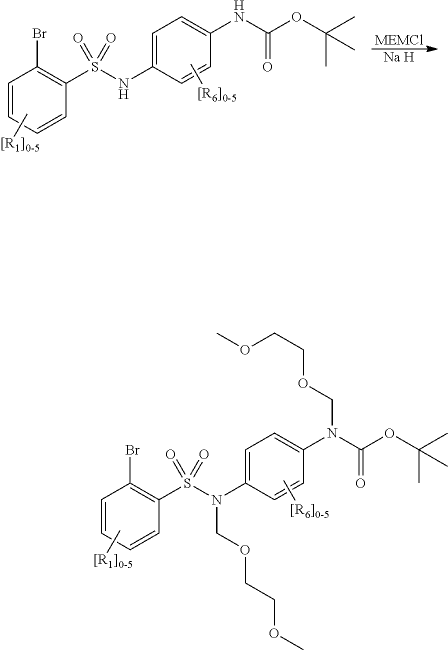

The appropriate sulfonamide was dissolved in DMF and cooled to 0° C. To this solution sodium hydride (3.2 eq.) was added and the reaction was stirred for 30 min. 2-Methoxyethoxymethyl chloride (MEMC1) (3.0 eq.) was added slowly to this solution and the reaction was stirred at room temperature until judged complete by LCMS. The mixture was concentrated under reduced pressure and the residue was dissolved in EtOAc. The organics were washed with H₂O (3×) and brine (1×), dried over Na₂SO₄ and concentrated over SiO₂. The mixture was purified via silica gel chromatography (0-100% EtOAc/Hexanes).

Procedure 54

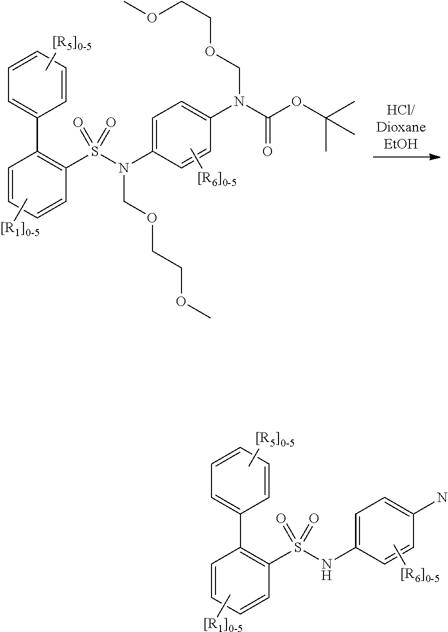

The appropriate MEM protected compound was dissolved in EtOH. A solution of HCl/dioxane (4 M, 10-25 eq.) was added and the mixture was refluxed until complete deprotection as judged by LCMS. The mixture was concentrated and used as is, alternatively the mixture was transferred to a separatory funnel containing DCM and the organics were washed with a saturated solution of NaHCO₃ (1×), H₂O (1×), brine (1×) and dried over MgSO₄. The combined organics were concentrated and purified via silica gel chromatography (0-20% DCM/MeOH).

Procedure 55

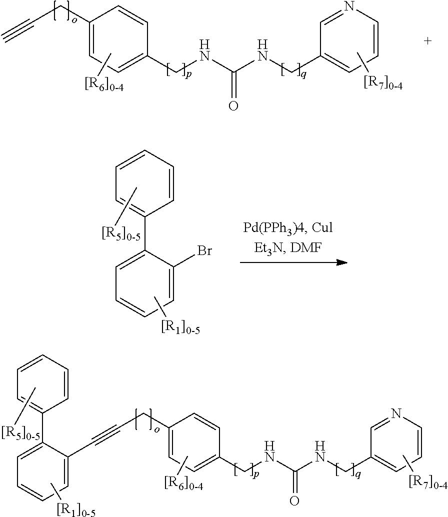

The appropriate aryl halide (1.0 eq.), 4-ethynylaniline (1.0 eq.), Pd(PPh₃)₄ (0.1 eq.) and CuI (0.05 eq.) were dissolved in DMF. The resulting mixture was sparged with nitrogen and Et₃N (1.5 eq.) was added. The mixture was heated to 80° C. overnight. Progress was monitored by LCMS and upon completion the reaction was concentrated over SiO₂ and purified via silica gel chromatography (0-50% EtOAc/Hexanes).

Procedure 56

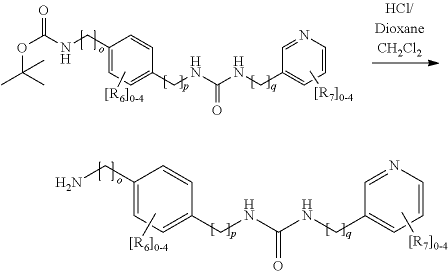

To a solution (0.2M) of the appropriate BOC protected amine (1.0 eq.) in CH₂Cl₂ was added HCl/Dioxane (3.0 eq.) dropwise. The mixture was stirred overnight at room temperature, concentrated and the residue was purified by silica gel chromatography.

Procedure 57

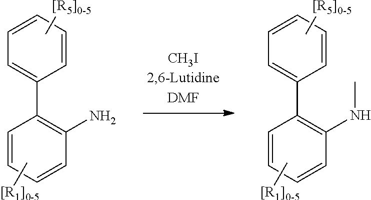

To a solution of the appropriate amine (2.95 mmol) and 2,6-lutidine (3.25 mmol) in DMF (0.2 M) was added methyl iodide (1 eq.) The mixture was stirred until complete by LCMS. The reaction mixture was concentrated and uses as is.

Procedure 58

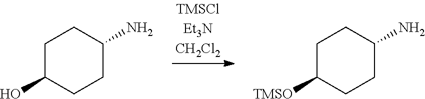

To a solution of the appropriate alcohol (1.0 eq.) in CH₂Cl₂ was added triethylamine (1.5 eq.) and trimethylsylyl chloride (TMSC1) (1.1 eq.). The mixture was stirred overnight at room temperature. If the reaction was not complete as judged by thin layer chromatography, TMSC1 (1.5 eq.) was added and the mixture was stirred until judged complete by TLC. The mixture was concentrated and purified by column chromatography.

Procedure 59

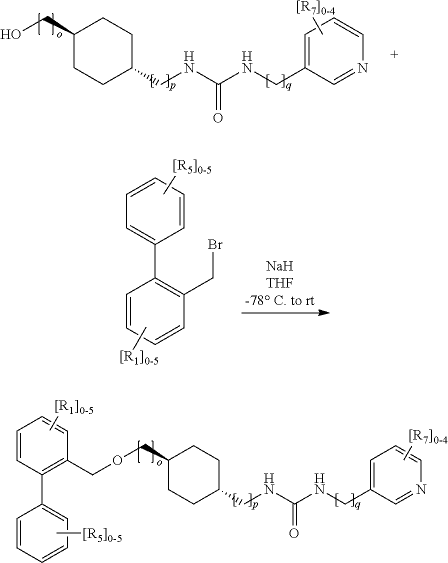

The appropriate alcohol (0.40 mmol) was dissolved in THF (2.0 mL) and cooled to −78° C. To the cold solution was added NaH (1.2 mmol). The reaction mixture was allowed to stir until no further gas evolution was visible. The appropriate bromide (1.1 eq.) was added, the acetone/dry ice bath was then removed and the mixture was allowed to warm to room temperature overnight. The mixture was concentrated and purified by silica gel column chromatography.

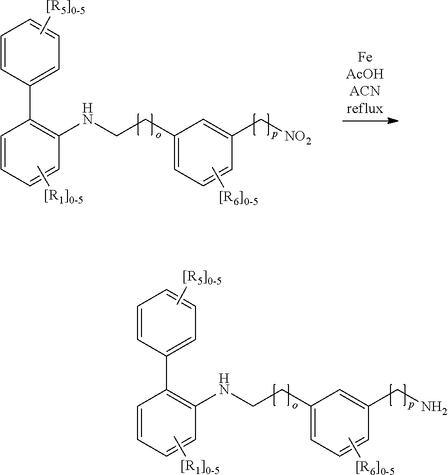

The appropriate nitro containing compound (1.0 eq.) was dissolved in a solution (0.2M) of acetonitrile and acetic acid (6.0 eq.). To this mixture was added a generous amount of iron powder (>5 eq.). The reaction mixture was refluxed until complete by TLC, approximately overnight. The reaction mixture was then filtered though celite, concentrated and purified by silica gel column chromatography.

Procedure 61

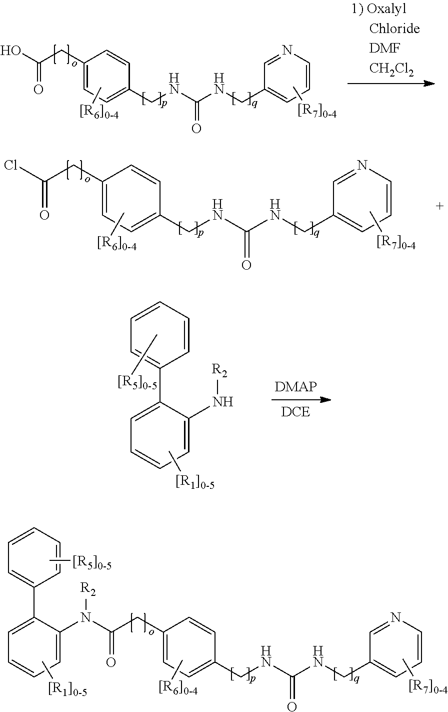

The appropriate carboxylic acid (1.0 eq.) was dissolved in CH₂Cl₂ (0.2 M) and cooled to 0° C. Oxalyl chloride (1.1 eq.) was added drop wise followed by a few drops of DMF. The solution was allowed to warm to room temperature, concentrated and the residue was dissolved in DCE (0.2 M). To this solution was added the appropriate amine/aniline (1.1 eq.) and a catalytic amount of DMAP. The mixture was refluxed overnight, concentrated and purified by silica gel column chromatography.

Procedure 62

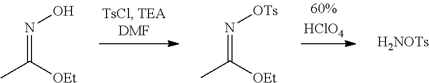

Tosyl Chloride (TsCl) (2.1 g, 11.00 mmol) was added to solution of ethyl N-hydroxyacetimidate (1.2 g, 11.6 mmol) and triethylamine (8.88 mL, 63.7 mmol) in DMF (20 mL) at 0° C. The reaction mixture was warmed to room temperature for 1 hour. The mixture was poured over ice-water (100 mL) and stirred. The yellow solid was filtered off, washed with cold water (3×50 mL). The filtered solid was treated with 60% HClO₄ for 1 hour and let cool to room temperature. Water was added to the reaction mixture (100 mL) and extracted with CH₂Cl₂ (50 mL) and washed with water (50 mL). The resulting solution of the product in CH₂Cl₂ was used as is.

Procedure 63

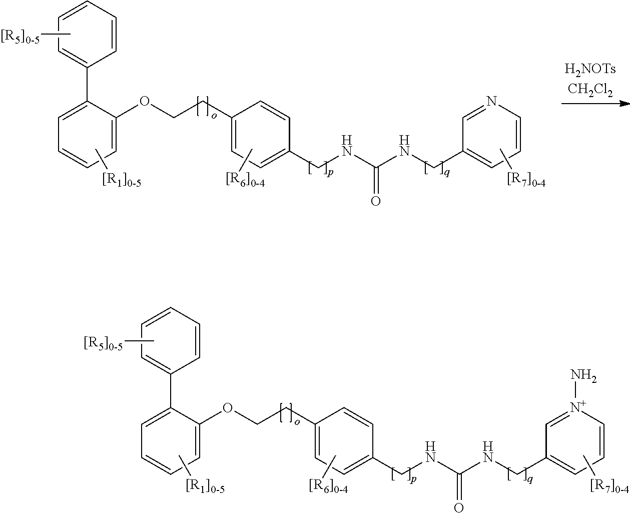

5 mL of solution of H₂NOTs in CH₂Cl₂ was added to an appropriate pyridyl compound (488 mmol) dissolved in 1 mL CH₂Cl₂ and stirred at room temperature for 3 hours. The mixture was concentrated and the residue was dissolved in MeOH and evaporated on celite. The mixture was purified by reverse phase column chromatography.

Procedure 64

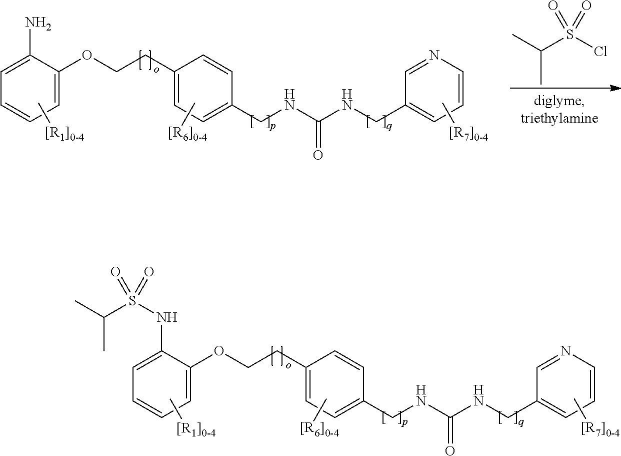

Triethylamine (2 eq.) was added to a stirring solution of the appropriate amine in diglyme (ca 0.2 M). The appropriate sulfonyl chloride (1.2 eq.) was added and the mixture and was stirred overnight at ambient temperature. Most of the diglyme was removed in vacuo. The reside was taken up in H₂O and extracted several times with ethyl acetate. The combined organic fractions were washed with water, brine, and dried with Na₂SO₄. The sulfonamide product was purified via silica gel chromatography.

Procedure 65

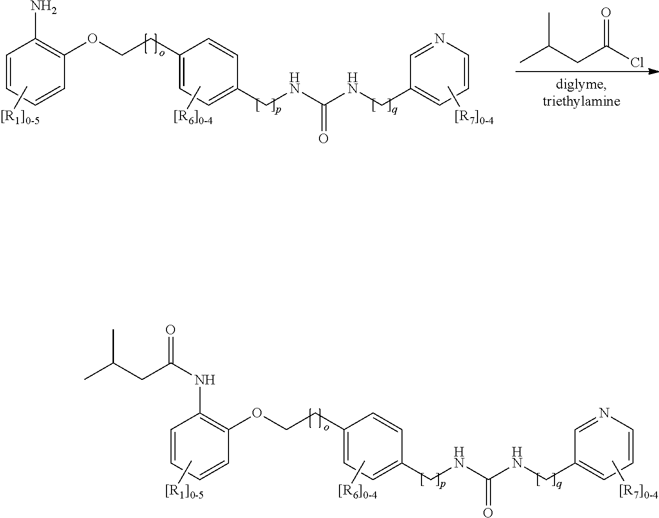

Triethylamine (2 eq.) was added to a stirring solution of the appropriate aniline in diglyme (ca 0.2 M). The desired acid chloride (1.2 eq.) was added and the mixture was stirred overnight at ambient temperature. Most of the diglyme was removed in vacuo. The reside was taken up in H₂O and extracted several times with ethyl acetate. The combined organic fractions were washed with water, brine, and dried with Na₂SO₄. The amide product was purified on silica gel chromatography.

Procedure 66

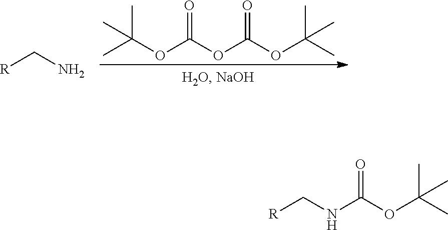

An aqueous solution of the appropriate amine (0.2 M) was treated 3M aqueous NaOH (3 eq.). After stirring for 10 min, Di-tert-butyl dicarbonate (Boc₂O) (1.2 eq.) was added. The mixture was stirred overnight at ambient temperature. The solution was slowly acidified to pH 3 with 3M aqueous HCl. The resulting white precipitate was collected by vacuum filtration, washed with H₂O, frozen, and dried by lyophilization. The material was used without further purification.

Procedure 67

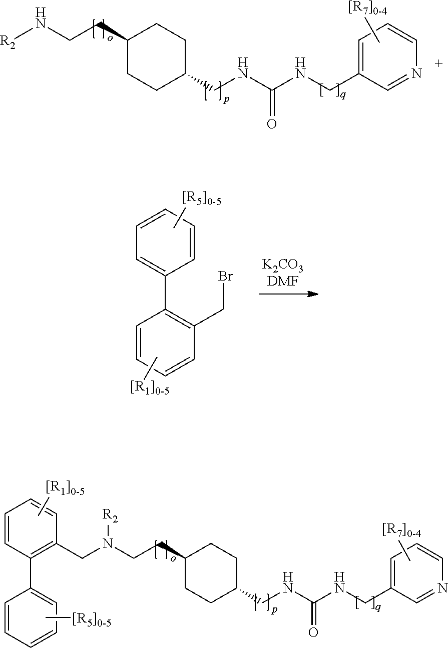

A solution of the appropriate amine (1 eq.) in DMF (0.1M) was treated with K₂CO₃ (5 eq.) and stirred for 30 min. The appropriate benzyl bromide was added and the reaction was stirred overnight at ambient temperature. Most of the DMF was removed in vacuo. The residue was dissolved in DCM and washed several times with H₂O. The organic layer was dried over anhydrous Na₂SO₄ (s). The crude material was purified by silica gel chromatography.

Procedure 68

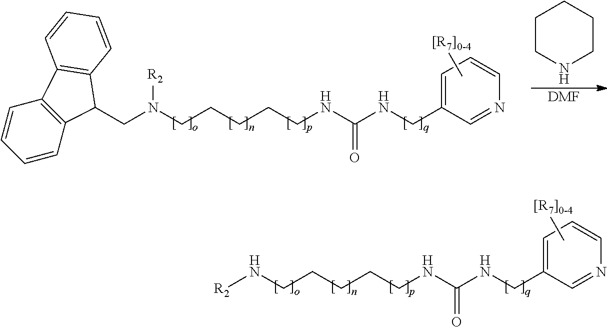

A solution of the appropriate Fmoc-protected amine in DMF (0.26 M) was treated with 2.4 eq. of piperidine and stirred overnight at ambient temperature. Most of the DMF was removed in vacuo and the residue was dissolved in H₂O and washed several times with EtOAc. The combined organic fractions were back-extracted with H₂O. The water was removed in vacuo and the desired compound was used as is.

Procedure 69

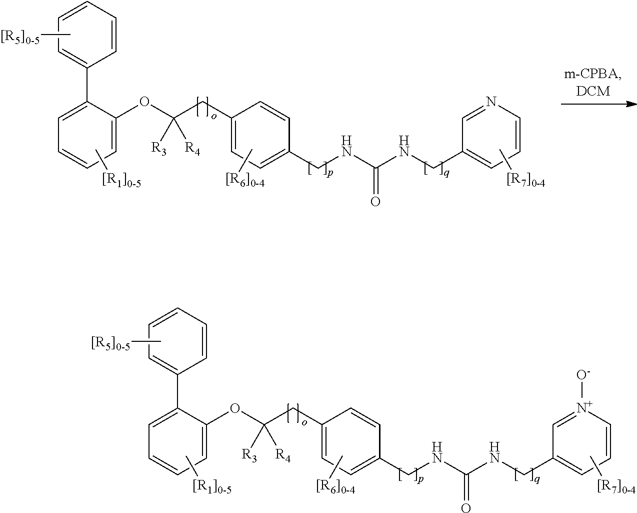

m-CPBA (2.2 eq.) was added to the desired pyridyl compound in DCM (0.2 M). The resulting mixture was stirred for 1-2 h. at rt. The mixture was concentrated and purified by silica gel chromatography.

Procedure 70

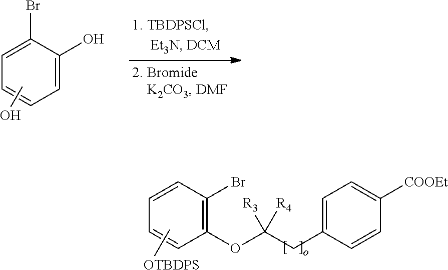

tert-Butyldiphenylsilyl chloride (TBDPSC1) (1.2 eq.) was added to the appropriate bisphenol (1 eq.) and Et₃N (1.5 eq.) in CH₂Cl₂ (0.2 M) and the solution is stirred at rt. for 2.5 h. The mixture was washed with H₂O, dried with Na₂SO₄, and concentrated. The appropriate bromide (1 eq.), K₂CO₃ (3 eq.), and DMF (0.5 M) are added and the solution was heated at 90° C. overnight. After 17 h. EtOAc was added and the solution was washed with 10% HCl, H₂O, and brine, dried with Na₂SO₄, and concentrated. The resulting oil was purified by silica gel chromatography.

Procedure 71

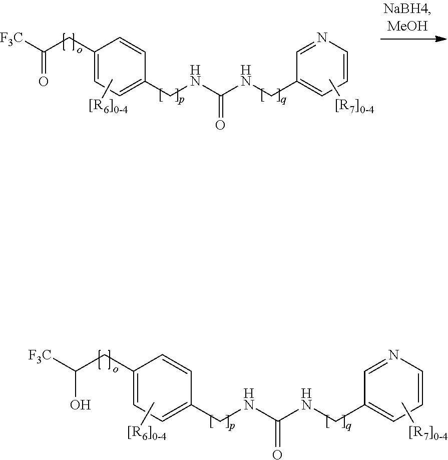

MeOH and NaBH₄ (1.2 eq.) were added to appropriate ketone or aldehyde and the reaction was stirred at rt. for 3 h. The reaction mixture was concentrated and purified by silica gel chromatography.

Procedure 72

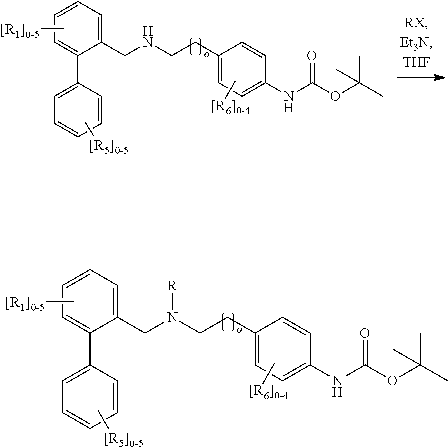

### The appropriate alkyl halide (3 eq.) was added to the appropriate amine and Et₃N (3 eq.) in THF. The solution was heated at reflux overnight. The solution was concentrated and purified by silica gel chromatography.

Procedure 73

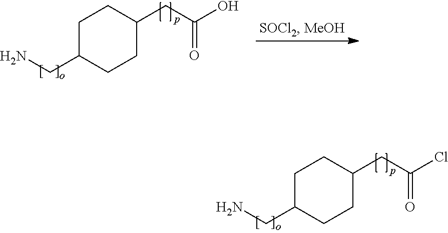

Thionyl chloride (2 eq.) was added drop wise to the appropriate acid in MeOH. The resulting solution was heated at reflux for 2-4 h. and concentrated. The product was carried on with out additional purification.

Procedure 74

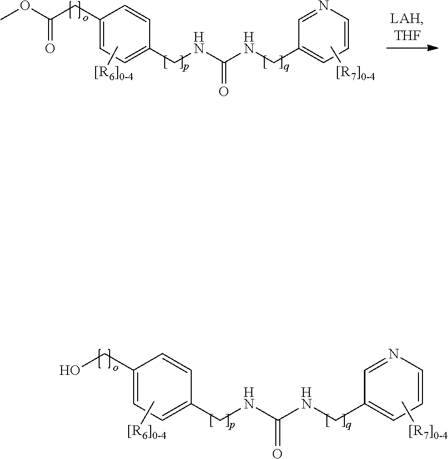

LiAlH₄ (1.2 eq., 2 M in THF) was added slowly to the appropriate ester (1 eq.) in THF and the solution is stirred at room temperatureovernight. Water, 10% NaOH, and more water was added dropwise, and the resulting slurry filtered over celite, washed with a large excess of ethyl acetate. The organics were dried with Na₂SO₄ and concentrated to yield the desired product.

Procedure 75

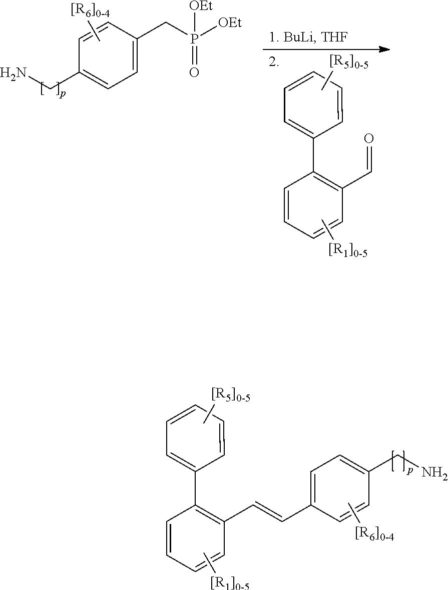

BuLi (1.2 eq, 2.5 M in hexanes) was added slowly to the appropriate phosphonate in THF at −78° C. The mixture was stirred at −78° C. for 15 minutes, the appropriate aldehyde (1.2 eq.) was added, and the solution was allowed to warm to rt. overnight. The reaction mixture was concentrated and purified by silica gel chromatography.

Procedure 76

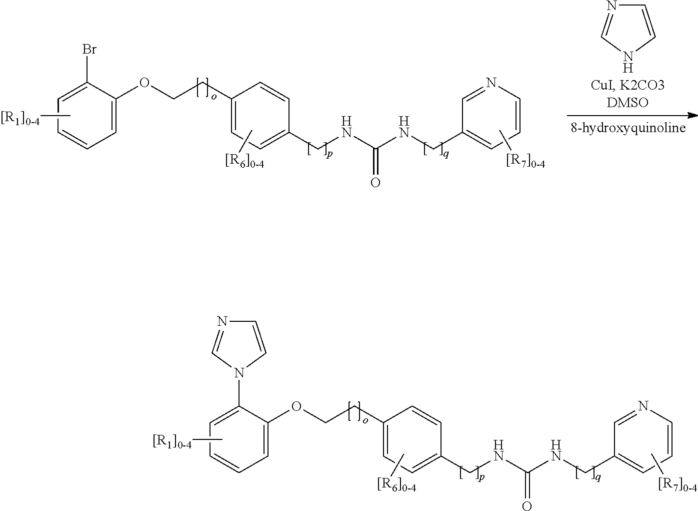

The appropriate aryl bromide (1 eq.), appropriate imidazole (1.2 eq.), CuI (0.2 eq.), 8-hydroxyquinoline (0.2 eq.), and K₂CO₃ were suspended in DMSO (1 M per ArBr) and purged with N₂ for 1-5 minutes. The solution was heated at 120° C. for 16-40 h., filtered, and purified by reverse phase silica gel chromatography.

Procedure 77

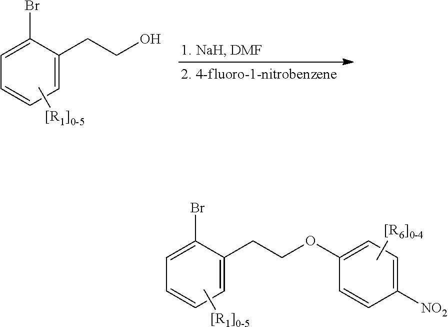

The appropriate alcohol (1 eq.) in DMF (0.5 M) was treated with NaH (1.2 eq., 60% w/w in mineral oil) and stirred at rt. for 20-30 min. 4-Fluoro-1-nitrobenzene (1.2 eq.) was added and the solution stirred at rt.-60° C. for 3-24 h. The reaction mixture was diluted with EtOAc, washed with 10% HCl, water, brine, dried with Na₂SO₄, and purified by silica gel chromatography.

Procedure 78

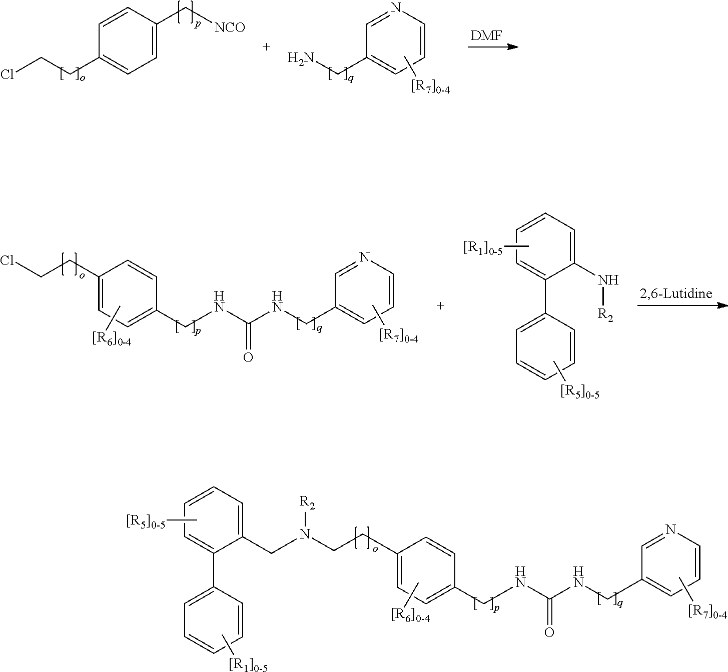

The appropriate amine (1 eq.) was added to the appropriate isocyanate (1 eq.) in DMF at 0° C. and the solution stirred at 0° C. for 90 minutes. The appropriate amine (1.2 eq.) and 2,6-lutidine (1.2 eq.) were added and the solution was stirred at 60° C. overnight, concentrated, and purified by silica gel chromatography.

Procedure 79

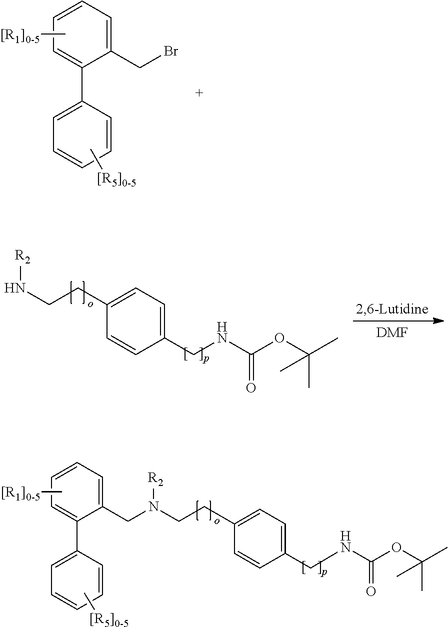

The appropriate benzyl bromide (1 eq.) was added to an appropriate amine (1 eq.) in DMF and the solution stirred at 80° C. overnight. The mixture was diluted with EtOAc, washed with sat. NaHCO₃, dried with Na₂SO₄, and concentrated. The product was carried on crude.

Procedure 80

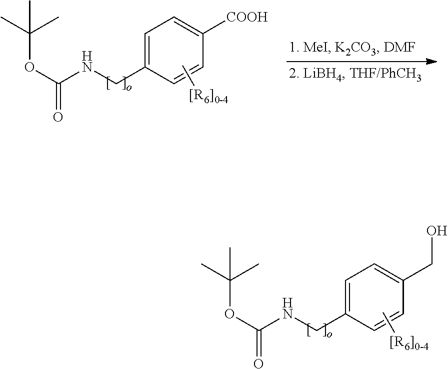

MeI (1.5 eq.) was added to the appropriate carboxylic acid (1 eq.) and K₂CO₃ (3 eq.) in DMF. The solution stirred at 60° C. for 3 h. EtOAc was added and washed with 10% HCl, water, brine, dried over Na₂SO₄, filtered and concentrated. THF and PhCH₃ were added, LiBH₄ (0.7 eq., 2 M in THF) was added slowly and the mixture was heated at 100° C. for 4 h. and then at rt. After 4 h. LiBH₄ (0.7 eq., 2 M in THF) was added. After 23 h. LiBH₄ (0.7 eq., 2 M in THF) was added and the solution heated to 100° C. After 6 h. at 100° C. the solution was cooled, diluted with water and EtOAC, and stirred at rt. for 1 h. The layers were separated, the organic layer dried with Na₂SO₄, concentrated, and purified by silica gel chromatography.

Procedure 81

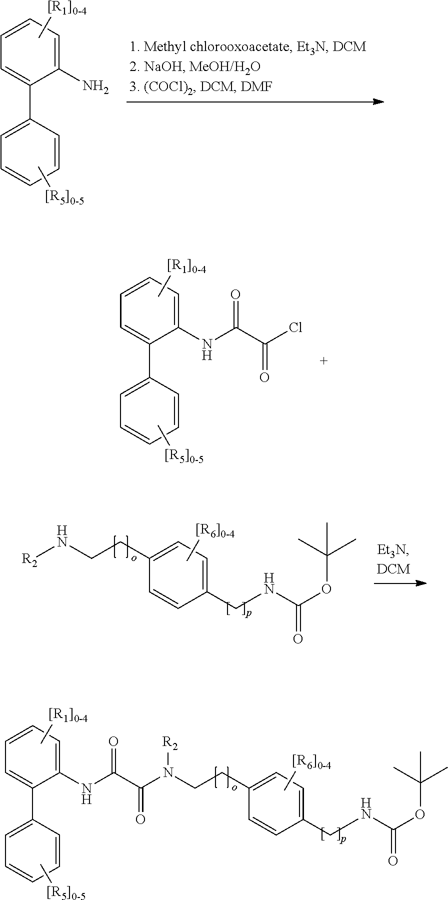

Methyl chlorooxoacetate (1.2 eq.) was added to the appropriate amine (1 eq.) and Et₃N (3 eq.) in DCM and the solution stirred at rt, for 1 h. The solution was diluted with DCM, washed with 10% HCl, dried with Na₂SO₄ and concentrated. Excess NaOH/H₂O and MeOH were added and the mixture heated to reflux for 1 h., the mixture was diluted with EtOAc, washed with 10% HCl, dried with Na₂SO₄ and concentrated. DCM and oxalyl chloride (2 eq.) were added followed by 1 drop of DMF. The solution was stirred at rt. for 30 min. and concentrated. DCM followed by Et₃N (3 eq.) and the appropriate amine (1 eq) were added and the solution stirred at room temperature for 1 h. The solution was diluted with DCM, washed with 10% HCl, dried with Na₂SO₄ and concentrated. The resulting material was carried on crude.

Procedure 82

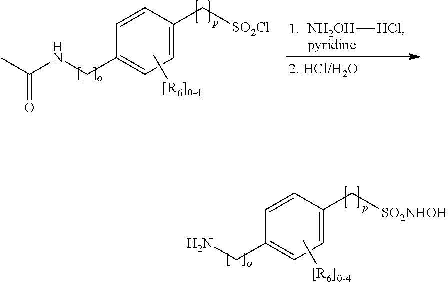

The appropriate sulfonyl chloride (1 eq.) was added slowly to hydroxylamine hydrochloride (2 eq.) in pyridine (0.8 M). The solution was stirred at rt. for 1 h., poured into 10% HCl, and cooled in the freezer overnight. The resulting solid was filtered, suspended in 10% HCl, and heated to reflux for 4 h. The solution was neutralized with 1 MNaOH, washed with EtOAc, and the organic layer dried with Na₂SO₄ and concentrated. The resulting material was carried on crude.

Procedure 83

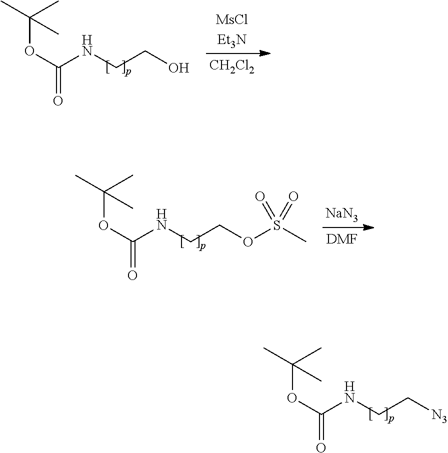

Methanesulfonyl chloride (1.1 eq.) was added to a solution of the appropriate protected amino alcohol (1.0 eq.) and triethylamine in CH₂Cl₂ at 0° C. The reaction mixture was allowed to warm to room temperature and stirred overnight. The mixture was filtered through celite and the filtrated was concentrated. The mesylate thus obtained was dissolved in DMF, NaN₃ (4.0 eq.) was added, and the resulting mixture was stirred overnight at 85° C. After cooling to room temperature, the reaction mixture was partioned between water and EtOAc, the layers were separated, and the aqueous layer was extracted with EtOAc (2×). The combined organic extracts were washed with water (1×), brine (1×), dried (Na₂SO₄), filtered, and concentrated. The azide thus obtained was used as is in subsequent reactions.

Procedure 84

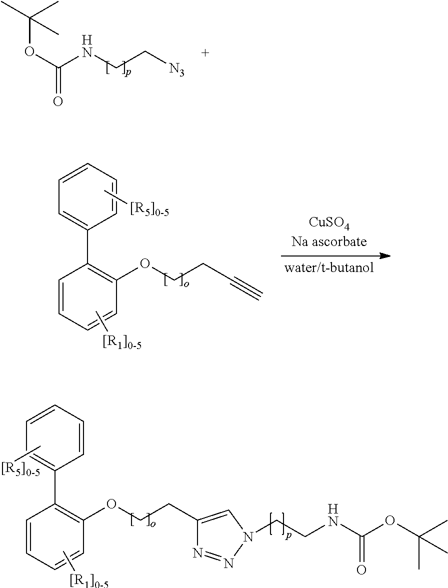

CuSO₄.5H₂O (0.01 eq.) was added to a suspension of the appropriate alkyl azide (1.0 eq.), appropriate alkyne (1.0 eq.), and sodium ascorbate (0.1 eq.) in water/t-butanol (1 mL:1 mL) and the resulting mixture was stirred overnight at 50° C. The reaction mixture was cooled to room temperature, the solvent was removed, and the resulting residue was purified by chromatography to yield the desired product.

Procedure 85

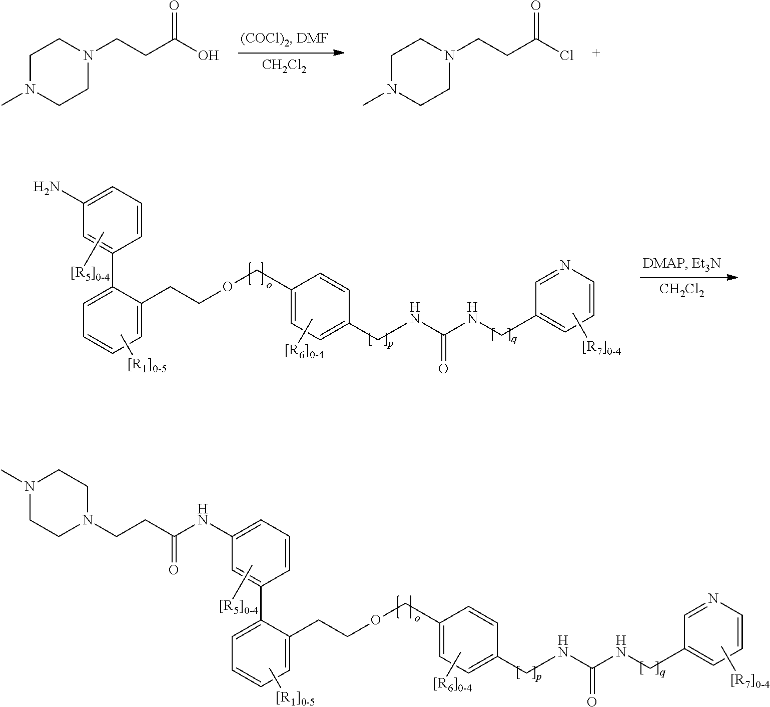

Oxallyl chloride (1.8 eq.) was added to a mixture of the appropriate acid (1.3 eq.) in CH₂Cl₂ at 0° C., followed by DMF (2-3 drops); the mixture was then stirred for 1 h at room temperature. The solvent was removed under vacuum, and the resulting residue was dissolved in CH₂Cl₂. To this mixture was added a solution of the appropriate aniline (1.0 eq.), Et₃N (1.5 eq.), and DMAP (catalytic amount) in CH₂Cl₂, and the resulting mixture was stirred overnight at room temperature. The reaction mixture was concentrated and purified by chromatography.

Procedure 86

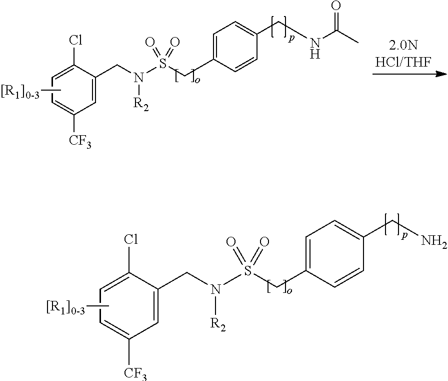

A mixture of the appropriate N-acetyl aniline (1.0 eq.) in 2.0 N HCl/THF (ca. 3 mL/1 mL) was stirred at reflux overnight. The mixture was cooled to room temperature and the solid precipitate was collected by filtration. The filter cake was washed with Et₂O, and dried under vacuum. In cases in which precipitate did not form upon cooling, the solvent was removed and the resulting residue was suspended in Et₂O/EtOAc. The resulting precipitate was collected by filtration and dried under vacuum.

Procedure 87

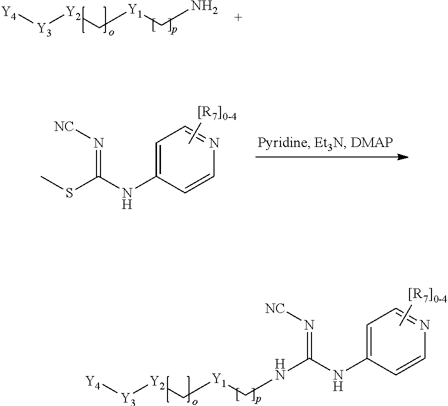

An appropriate amine, methyl N′-cyano-N-(4-pyridyl)carbamimidothioate, Et₃N, and DMAP (cat.) were heated in pyridine at reflux overnight. The solution was cooled and was added to Et₂O. The resulting residue was isolated by filtration or decantation and purified by silica gel chromatography or RP-HPLC.

Procedure 88

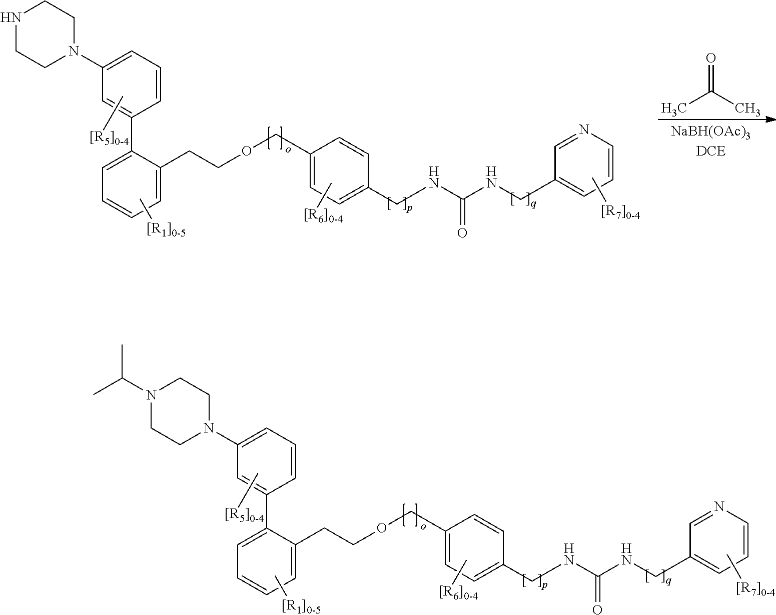

To the appropriately substituted piperazine (0.074 mmol) in dichloroethane (2 mL) was added acetone (0.74 mmol). After stirring for 5 minutes sodium triacetoxyborohydride (0.15 mmol) was added to the mixture. The reaction was allowed to stir for 24 hrs then quenched with addition of MeOH (5 mL). The reaction was concentrated and purified via reverse phase (RP)-HPLC.

Procedure 89

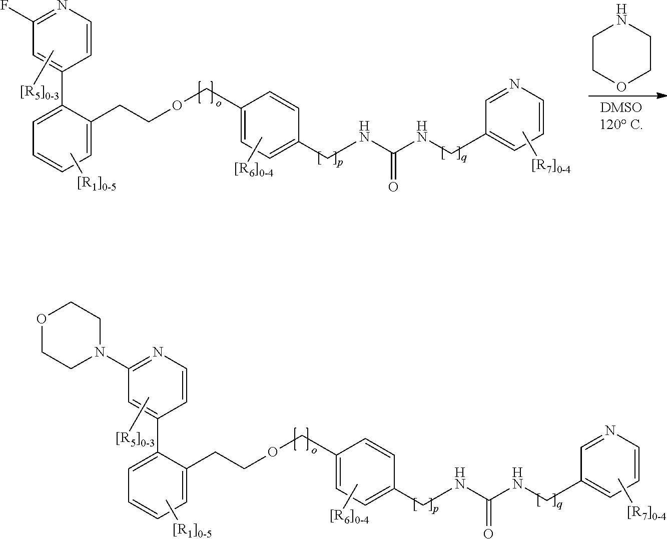

To the appropriately substituted fluoro-pyridyl intermediate (0.072 mmol) in dimethylsulfoxide (1 mL) was added morpholine (0.72 mmol). The reaction was heated to 100° C. and allowed to stir for 24 hrs. The reaction was concentrated and purified via reverse phase (RP)-HPLC.

Procedure 90

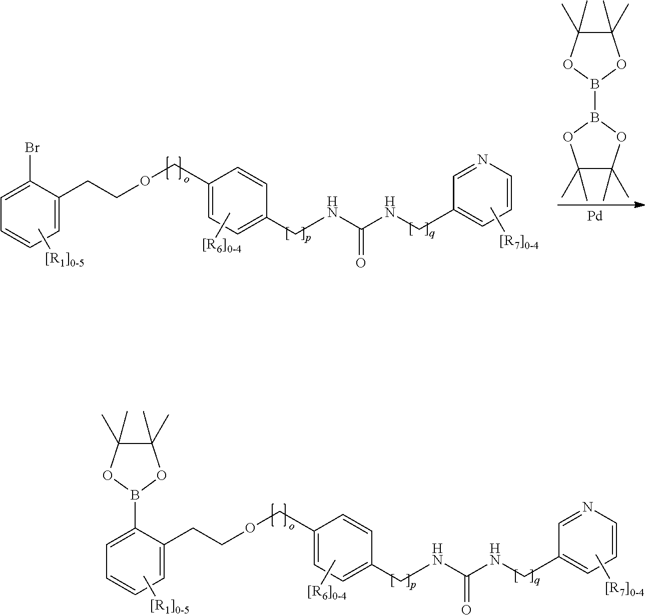

To the appropriate aryl bromide (3.6 mmol) in DMF (12 mL) was added bis(pinacolato)diboron (7.3 mmol), 1,1′-Bis(diphenylphosphino)ferrocene-palladium(II)dichloride dichloromethane complex (0.36 mmol) and potassium acetate. The reaction was stirred and heated at 80° C. overnight. The reaction was concentrated and purified by silica gel chromatography (0-15% MeOH in DCM) to afford the desired compound.

Procedure 91

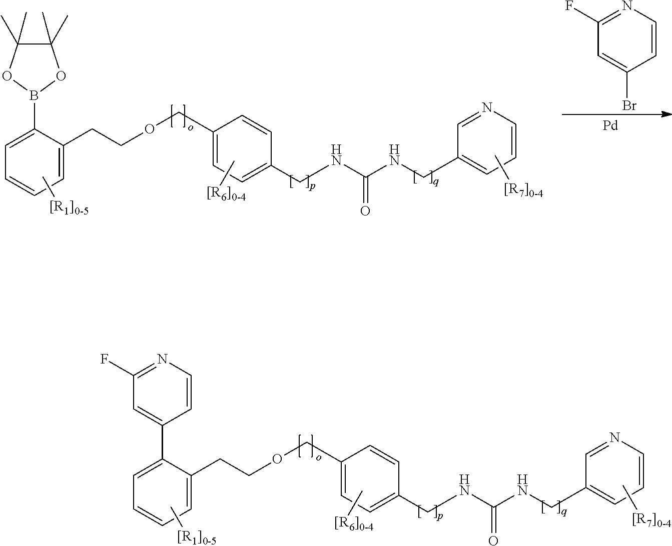

To the appropriate boronate ester (0.2 mmol) in DMF (1.5 mL) was added tetrakis(triphenyl-phosphine) palladium (0.02 mmol), and 5-bromo-2-fluoropyridine (0.3 mmol). Nitrogen was bubbled through the reaction for 5 min and sodium carbonate (250 μL, 2M) was added. Nitrogen was again bubbled through the reaction. The reaction was then stirred with heating at 90° C. overnight. The solvent was removed under vacuum and the residue was partitioned between water and DCM. The organic layer was dried (MgSO₄), concentrated and purified by C18 chromatography to afford the desired product.

Exemplary compounds of the present invention are shown in Tables 1-6. Tables 1 and 3 are separated into an “A” and “B”. The “A” tables show the structure, name, and NMR data (if generated) for a particular example compound. Compound names were generated using ACD Labs IUPAC nomenclature software version 12.00 (Toronto, Ontario, Canada).

The “B” tables show the molecular weight found using High Resolution Mass Spectrometry (“HRMS”) and also lists the Synthetic Procedures used to make the particular example compound. In some instances, the Synthetic Procedure listed is similar to the procedure actually used to make a particular example compound, rather than the actual procedure used. Each of the example compounds were synthesized using commercially available starting materials that are well known in the art. The synthetic procedures used to make the example compounds of Tables 5 and 6 are not specifically identified, however, the procedures were used similar to those used to make the example compounds of Tables 1-4.

### Example Compounds

TABLE 1A

| Ex- | | |
|---|---|---|
| ample | | ¹H NMR Data (400 MHz, |
| Number | Structure | DMSO-d6) |
| 1 | 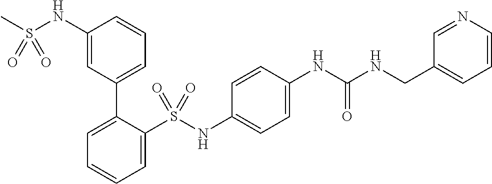 [SMILES: CS(=O)(=O)Nc1cccc(-c2ccccc2S(=O)(=O)Nc2ccc(NC(=O)NCc3cccnc3)cc2)c1] | 9.83 (s, 1H), 9.78 (s, 1H), 8.67-8.60 (m, 3H), 8.02 (d, 1H), 7.93 (dd, 1H), 7.60-7.37 (m, 3H), 7.37-7.29 (m, 3H), 7.23-7.17 (m, 5H), 6.95 (dd, 1H), 6.83 (d, 2H), 6.77 (t, 1H), 4.35 (d, 2H), 3.03 (s, 3H) |
| 2 | 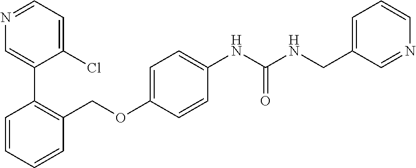 [SMILES: O=C(NCc1cccnc1)Nc1ccc(OCc2ccccc2-c2cnccc2Cl)cc1] | 8.68-8.77 (m, 2 H) 8.61 (s, 1 H) 8.51-8.54 (m, 2 H) 8.28 (d, 1 H) 7.86 (dd, 1 H) 7.68 (d, 1 H) 7.60-7.65 (m, 1 H) 7.46-7.53 (m, 2 H) 7.28- 7.33 (m, 1 H) 7.16-7.23 (m, 2 H) 6.78 (t, 1 H) 6.64-6.68 (m, 2 H) 4.68-4.87 (m, 2 H) 4.41 (d, 2 H). |
| 3 | 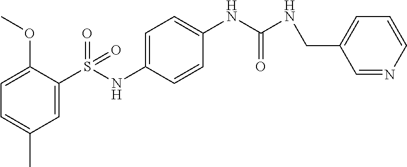 [SMILES: COc1ccc(C)cc1S(=O)(=O)Nc1ccc(NC(=O)NCc2cccnc2)cc1] | 9.57 (s, 1H), 8.51 (s, 1H), 8.49-8.47 (m, 1H), 8.45-8.42 (m, 1H), 7.69-7.64 (m, 1H), 7.45 (d, 1H), 7.36-7.31 (m, 2H), 7.19 (d, 2H), 7.05 (d, 1H), 6.93 (d, 2H), 6.64 (t, 1H), 4.26 (d, 2H), 3.87 (s, 3H), 2.21 (s, 3H) |
| 4 | 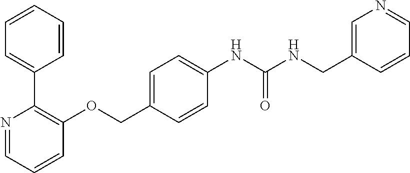 [SMILES: O=C(NCc1cccnc1)Nc1ccc(COc2cccnc2-c2ccccc2)cc1] | 8.72 (s, 1H), 8.52 (br s, 1H), 8.45 (d, 1H), 8.26 (dd, 1H), 7.90 (dd, 2H), 7.65 (dd, 2H), 7.42-7.28 (m, 8H), 6.76 (t, 1H), 5.11 (s, 2H), 4.31 (d, 2H) |
| 5 | 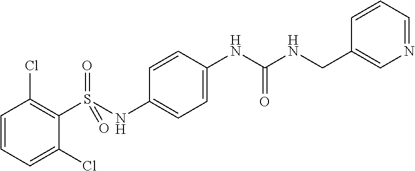 [SMILES: O=C(NCc1cccnc1)Nc1ccc(NS(=O)(=O)c2c(Cl)cccc2Cl)cc1] | 10.46 (s, 1H), 8.58 (s, 1H), 8.49 (s, 1H), 8.46-8.40 (m, 1H), 7.67 (d, 1H), 7.60 (d, 2H), 7.55-7.47 (m, 1H), 7.38- 7.30 (m, 1H), 7.26 (d, 2H), 6.97 (d, 2H), 6.68 (t, 1H), 4.27 (d, 2H) |
| 6 | 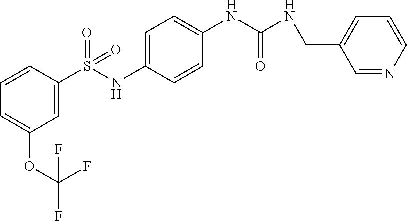 [SMILES: O=C(NCc1cccnc1)Nc1ccc(NS(=O)(=O)c2cccc(OC(F)(F)F)c2)cc1] | 10.10 (s, 1H), 8.61 (s, 1H), 8.50 (d, 1H), 8.46-8.43 (m, 1H), 7.71-7.62 (m, 4H), 7.60 (bs, 1H), 7.37-7.32 (m, 1H), 7.27 (d, 2H), 6.91 (d, 2H), 6.67 (t, 1H), 4.28 (d, 2H) |
| 7 | 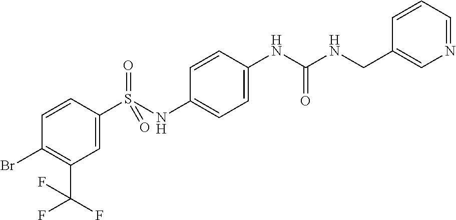 [SMILES: O=C(NCc1cccnc1)Nc1ccc(NS(=O)(=O)c2ccc(Br)c(C(F)(F)F)c2)cc1] | 10.18 (s, 1H), 8.81 (s, 1H), 8.71-8.62 (m, 2H), 8.13 (d, 1H), 8.08 (d, 1H), 8.01 (s, 1H), 7.80-7.69 (m, 2H), 7.29 (d, 2H), 6.92 (d, 2H), 6.92 (t, 1H), 4.38 (d, 2H) |
| 8 | 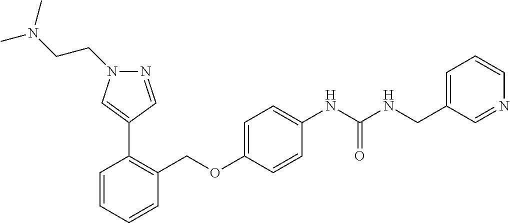 [SMILES: CN(C)CCn1cc(-c2ccccc2COc2ccc(NC(=O)NCc3cccnc3)cc2)cn1] | 8.70 (s, 1 H) 8.67 (s, 1 H) 8.63 (d, 1 H) 8.02-8.10 (m, 2 H) 7.75 (s, 1 H) 7.67 (dd, 1 H) 7.56 (d, 1 H) 7.45-7.49 (m, 1 H) 7.38-7.44 (m, 1 H) 7.29-7.36 (m, 3 H) 6.84- 6.94 (m, 3 H) 4.99 (s, 2 H) 4.54 (t, 2 H) 4.38 (d, 2 H) 3.57 (t, 2 H) 2.80 (s, |
| 9 | 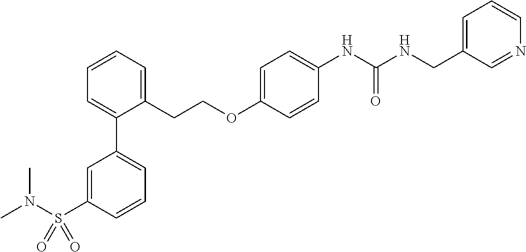 [SMILES: CN(C)S(=O)(=O)c1cccc(-c2ccccc2CCOc2ccc(NC(=O)NCc3cccnc3)cc2)c1] | 8.51 (d, 1H), 8.45 (dd, 1H), 8.39 (s, 1H), 7.77-7.74 (m, 3H), 7.72-7.69 (m, 2H), 7.51 (dd, 1H), 7.43-7.33 (m, 3H), 7.266 (dd, 1H), 7.21 (d, 2H), 6.66 (d, 2H), 6.59 (t, 1H), 4.29 (d, 2H), 4.00 (t, 2H), 2.96 (t, 2H), 2.59 (s, 6H) |
| 10 | 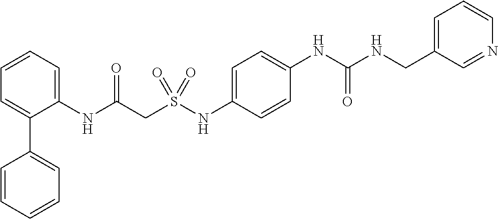 [SMILES: O=C(CS(=O)(=O)Nc1ccc(NC(=O)NCc2cccnc2)cc1)Nc1ccccc1-c1ccccc1] | 9.62 (s, 2H), 8.59 (s, 1H), 8.52 (d, 1H), 8.45 (dd, 1H), 7.70 (m, 1H), 7.64 (d, 1H), 7.42 (s, 2H), 7.41 (d, 2H), 7.39-7.26 (m, 8H), 7.08 (d, 2H), 6.67 (d, 1H), 4.31 (d, 2H), 3.98 (s, 2H) |
| 11 | 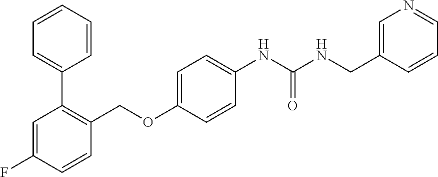 [SMILES: O=C(NCc1cccnc1)Nc1ccc(OCc2ccc(F)cc2-c2ccccc2)cc1] | 8.67 (s, 1H), 8.52 (d, 1H), 8.45 (dd, 1H), 7.70 (dt, 1H), 7.54 (d, 2H), 7.42-7.31 (m, 6H), 7.22-7.15 (m, 5H), 6.73 (t, 1H), 4.99 (s, 2H), 4.31 (d, 2H) |
| 12 | 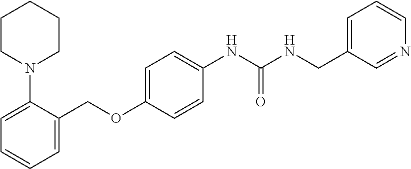 [SMILES: O=C(NCc1cccnc1)Nc1ccc(OCc2ccccc2N2CCCCC2)cc1] | 8.96 (s, 1H), 8.73-8.69 (m, 1H), 8.68-8.65 (m, 1H), 8.14 (d, 1H), 7.76-7.71 (m, 1H), 7.46 (d, 2H), 7.43-7.36 (m, 3H), 7.32-7.23 (m, 2H), 7.08- 6.98 (m, 2H), 5.15 (s, 2H), 4.41 (d, 2H), 3.44-3.21 (m, 4H), 1.89-1.72 (m, 4H), 1.61- 1.51 (m, 2H) |
| 13 | 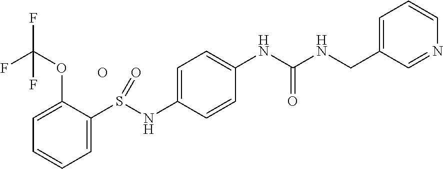 | 10.18 (s, 1H), 8.51-8.41 (m, 2H), 7.86 (d, 1H), 7.77-7.63 (m, 2H), 7.57-7.45 (m, 2H), 7.37-7.31 (m, 1H), 7.24 (d, 2H), 6.92 (d, 2H), 6.66 (t, 1H), 4.28 (d, 2H) |
| 14 | 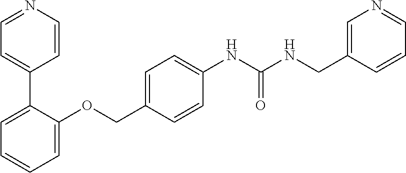 [SMILES: O=C(NCc1cccnc1)Nc1ccc(COc2ccccc2-c2ccncc2)cc1] | 8.92 (s, 1H), 8.79 (dd, 1H), 8.68 (d, 1H), 8.64 (dd, 1H), 8.10 (dt, 1H), 8.01 (d, 2H), 7.70 (dd, 1H), 7.55-7.50 (m, 2H), 7.40 (d, 2H), 7.34-7.28 (m, 2H), 7.15 (td, 1H), 6.96 (t, 1H), 5.11 (s, 2H), 4.40 (d, 2H) |
| 15 | 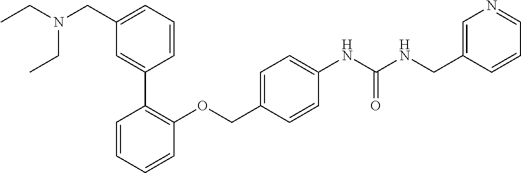 [SMILES: CCN(CC)Cc1cccc(-c2ccccc2OCc2ccc(NC(=O)NCc3cccnc3)cc2)c1] | 9.39 (bs, 1H), 8.95 (s, 1H), 8.68 (s, 1H), 8.65 (d, 1H), 8.10 (d, 1H), 7.71 (dd, 1H), 7.63 (d, 2H), 7.50 (t, 1H), 7.45 (t, 1H), 7.37 (m, 4H), 7.23 (m, 2H), 7.07 (t, 1H), 7.02 (t, 1H), 5.02 (s, 1H), 4.39 (d, 2H), 4.29 (d, 2H), 3.03 (m, 4H), 1.18 (t, 6H) |
| 16 | 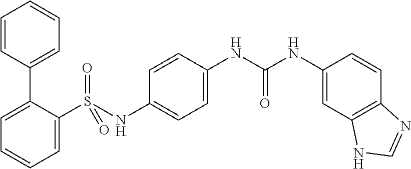 [SMILES: O=C(Nc1ccc(NS(=O)(=O)c2ccccc2-c2ccccc2)cc1)Nc1ccc2nc[nH]c2c1] | 9.88 (s, 1H), 9.26 (s, 1H), 9.22 (s, 1H), 8.87 (s, 1H), 8.17 (m, 1H), 7.99-7.97 (m, 1H), 7.71 (d, 1H), 7.67-7.55 (m, 2H), 7.42-7.22 (m, 9H), 6.89 (d, 2H) |
| 17 | 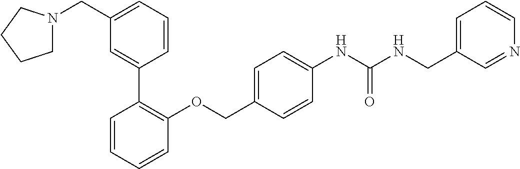 [SMILES: O=C(NCc1cccnc1)Nc1ccc(COc2ccccc2-c2cccc(CN3CCCC3)c2)cc1] | 8.74 (s, 1H), 8.54-8.49 (m, 1H), 8.48-8.42 (m, 1H), 7.73- 7.62 (m, 1H), 7.47 (s, 1H), 7.42-7.16 (m, 11H), 7.06-7.00 (m, 1H), 6.78 (t, 1H), 5.00 (s, 2H), 4.31 (d, 2H), 3.56 (s, 2H), 2.42-2.36 (m, 2H), 1.90 (s, 2H), 1.72-1.58 (m, 4H) |
| 18 | 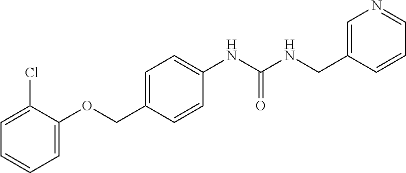 [SMILES: O=C(NCc1cccnc1)Nc1ccc(COc2ccccc2Cl)cc1] | 8.72 (s, 1H), 8.52 (d, 1H), 8.45 (dd, 1H), 7.71 (dt, 1H), 7.42 (d, 3H), 7.38-7.21 (m, 5H), 6.95 (td, 1H), 6.74 (t, 1H), 5.09 (s, 2H), 4.32 (d, 2H) |
| 19 | 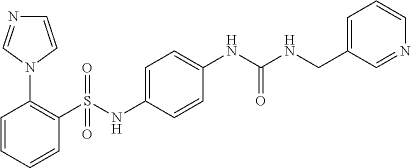 [SMILES: O=C(NCc1cccnc1)Nc1ccc(NS(=O)(=O)c2ccccc2-n2ccnc2)cc1] | 10.33 (s, 1H), 9.30 (s, 1H), 8.78 (s, 1H), 8.67 (bs, 1H), 8.05 (d, 1H), 7.97 (dd, 1H), 7.93-7.49 (m, 6H), 7.30 (d, 2H), 6.90 (d, 2H), 6.83 (t, 1H), 4.37 (d, 2H) |
| 20 | 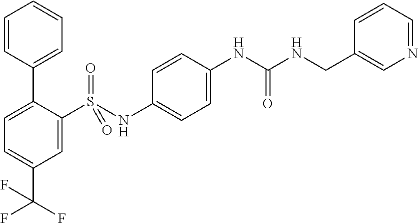 [SMILES: O=C(NCc1cccnc1)Nc1ccc(NS(=O)(=O)c2cc(C(F)(F)F)ccc2-c2ccccc2)cc1] | 10.08 (s, 1H), 8.77 (s, 1H), 8.66-8.63 (m, 1H), 8.63-8.60 (m, 1H), 8.27 (s, 1H), 8.08- 7.96 (m, 2H), 7.69-7.62 (m, 1H), 7.54 (d, 1H), 7.45-7.37 (m, 4H), 7.29-7.20 (m, 4H), 6.84 (d, 2H), 4.36 (d, 2H) |
| 21 | 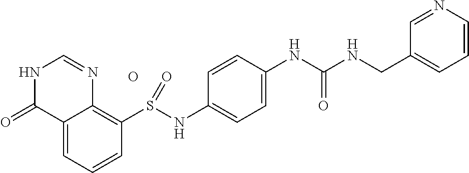 | 9.57 (s, 1H), 8.63-8.62 (m, 2H), 8.60 (d, 1H), 8.31 (dd, 1H), 8.29 (s, 1H), 8.19 (dd, 1H), 8.02 (d, 1H), 7.65 (dd, 1H), 7.56 (t, 1H), 7.15 (d, 2H), 6.90 (d, 2H), 6.74 (t, 1H), 4.32 (d, 2H) |
| 22 | 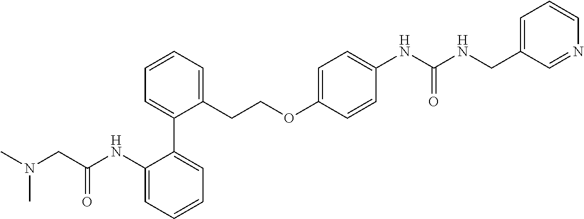 [SMILES: CN(C)CC(=O)Nc1ccccc1-c1ccccc1CCOc1ccc(NC(=O)NCc2cccnc2)cc1] | 9.87 (br s, 1H), 8.64 (d, 1H), 8.61 (dd, 1H), 8.57 (s, 1H), 8.02 (dt, 1H), 7.64 (dd, 1H), 7.52 (dd, 1H), 7.48-7.44 (m, 2H), 7.40-7.27 (m, 4H), 7.21 (d, 2H), 7.13 (d, 1H), 6.77 (t, 1H), 6.62 (d, 2H), 4.36 (d, 2H), 3.93 (t, 2H), 3.85 (s, 2H), 2.89-2.82 (m |
| 23 | 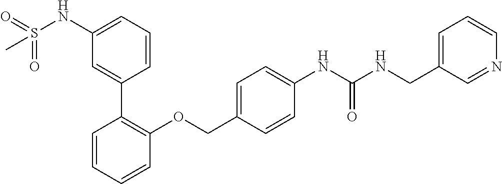 [SMILES: CS(=O)(=O)Nc1cccc(-c2ccccc2OCc2ccc(NC(=O)NCc3cccnc3)cc2)c1] | 9.78 (s, 1H), 8.85 (s, 1H), 8.69 (s, 1H), 8.64 (d, 1H), 8.10 (d, 1H), 7.73-7.67 (m, 1H), 7.45-7.12 (m, 11H), 7.03 (t, 1H), 6.91-6.85 (m, 1H), 5.01 (s, 2H), 4.40 (d, 2H), 2.89 (s, 3H) |
| 24 | 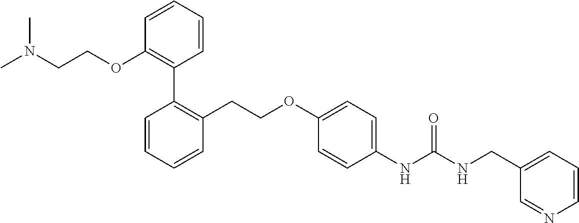 [SMILES: CN(C)CCOc1ccccc1-c1ccccc1CCOc1ccc(NC(=O)NCc2cccnc2)cc1] | 8.51 (s, 1H), 8.49 (s, 1H), 8.45 (s, 1H), 7.69 (d, 1H), 7.06-7.43 (m, 11H), 6.66 (t, 1H), 6.57 (dt, 2H), 4.29 (d, 2H), 4.14 (q, 2H), 3.88 (m, 2H), 3.34 (s, 2H), 2.85 (2H), 2.81 (m, 2H), 2.26 (s, 6H) |
| 25 | 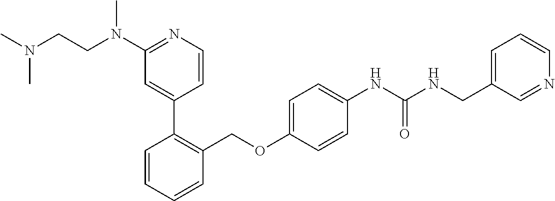 [SMILES: CN(C)CCN(C)c1cc(-c2ccccc2COc2ccc(NC(=O)NCc3cccnc3)cc2)ccn1] | 8.79 (s, 1 H) 8.71 (s, 1 H) 8.68 (d, 1 H) 8.10-8.19 (m, 2 H) 7.77 (dd, 1 H) 7.60- 7.64 (m, 1 H) 7.45-7.53 (m, 2 H) 7.37-7.41 (m, 1 H) 7.27- 7.30 (m, 2 H) 7.02 (t, 1 H) 6.73-6.84 (m, 4 H) 4.92 (s, 2 H) 4.40 (d, 2 H) 3.88 (t, 2 H) 3.27 (t, 2 H) 2. |
| 26 | 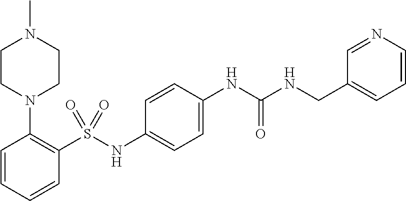 [SMILES: CN1CCN(c2ccccc2S(=O)(=O)Nc2ccc(NC(=O)NCc3cccnc3)cc2)CC1] | 8.55 (s, 1H), 8.48 (s, 1H), 8.43 (d, 1H), 7.78 (d, 1H), 7.66 (d, 1H), 7.55 (t, 1H), 7.42 (d, 1H), 7.33 (dd, 1H), 7.21 (t, 1H), 7.17 (d, 2H), 6.86 (d, 2H), 6.68 (t, 1H), 4.26 (d, 2H), 2.85 (br s, 4H), 1.79 (br s, 4H), 1.53 (br s, 2H). |
| 27 | 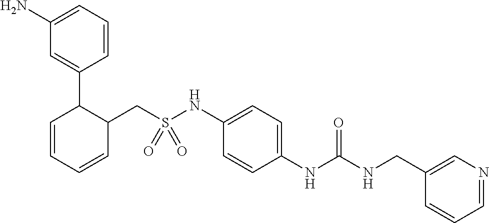 [SMILES: Nc1cccc(C2C=CC=CC2CS(=O)(=O)Nc2ccc(NC(=O)NCc3cccnc3)cc2)c1] | 9.68 (s, 1H), 8.75 (s, 1H), 8.70 (s, 1H), 8.65 (d, 1H), 8.12 (d, 1H), 7.72 (dd, 1H), 7.50-7.45 (m, 1H), 7.43-7.39 (m, 2H), 7.27 (d, 2H), 7.25- 7.21 (m, 1H), 7.18 (dd, 1H), 6.91 (d, 2H), 6.88-6.75 (m, 4H), 4.41 (d, 2H), 4.36 (s, 2H) |
| 28 | 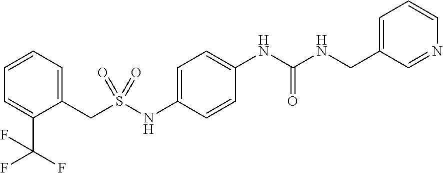 [SMILES: O=C(NCc1cccnc1)Nc1ccc(NS(=O)(=O)Cc2ccccc2C(F)(F)F)cc1] | (400 MHz, MeOH-d4) 8.78 (s, 1H), 8.70 (s, 1H), 8.48 (d, 1H), 7.96 (m, 1H), 7.71 (d, 1H), 7.66 (d, 1 h), 7.61 (dd, 1H), 7.51 (dd, 1H), 7.34 (d, 2H), 7.14 (d, 2H), 4.56 (s, 1H), 4.54 (s, 1H) |
| 29 | 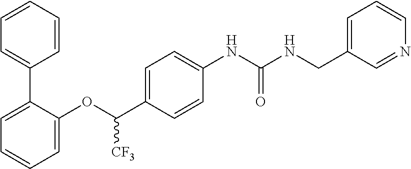 [SMILES: O=C(NCc1cccnc1)Nc1ccc(C(Oc2ccccc2-c2ccccc2)C(F)(F)F)cc1] | 8.79 (s, 1H), 8.50 (d, 1H), 8.44 (dd, 1H), 7.68 (d, 1H), 7.65-7.19 (m, 14H), 7.06 (t, 2H), 6.75 (t, 1H), 6.17 (q, 1H), 4.31 (d, 2H) |
| 30 | 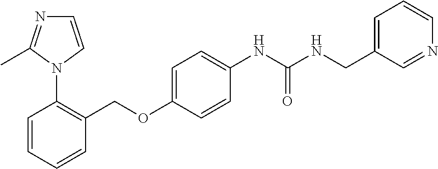 [SMILES: Cc1nccn1-c1ccccc1COc1ccc(NC(=O)NCc2cccnc2)cc1] | 8.49 (s, 1H), 7.87 )s, 1H), 7.81 (d, 1H), 7.77-7.64 (m, 4H), 7.46 (bs, 1H), 7.27 (d, 2H), 6.75 (d, 2H), 6.65 (t, 1H), 4.90 (s, 2H), 4.32 (d, 2H), 2.38 (s, 3H) |
| 31 | 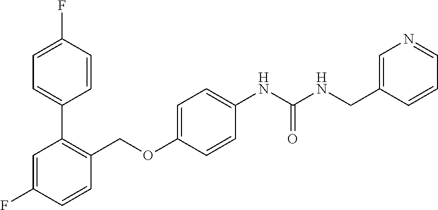 [SMILES: O=C(NCc1cccnc1)Nc1ccc(OCc2ccc(F)cc2-c2ccc(F)cc2)cc1] | 8.66 (s, 1H), 8.52 (d, 1H), 8.45 (dd, 1H), 7.70 (dt, 1H), 7.60-7.55 (m, 2H), 7.38-7.33 (m, 3H), 7.26-7.15 (m, 7H), 6.72 (t, 1H), 4.99 (s, 2H), 4.31 (d, 2H) |
| 32 | 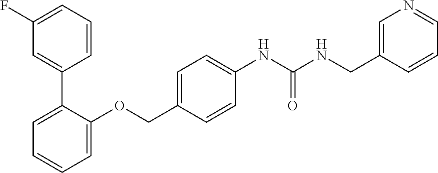 [SMILES: O=C(NCc1cccnc1)Nc1ccc(COc2ccccc2-c2cccc(F)c2)cc1] | 8.56 (s, 1H), 8.70 (d, 1H), 8.65 (dd, 1H), 8.14 (dt, 1H), 7.73 (dd, 1H), 7.46-7.34 (m, 7H), 7.26-7.20 (m, 3H), 7.16- 7.10 (m, 1H), 7.05 (td, 1H), 6.90 (t, 1H), 5.04 (s, 2H), 4.40 (d, 2H) |
| 33 | 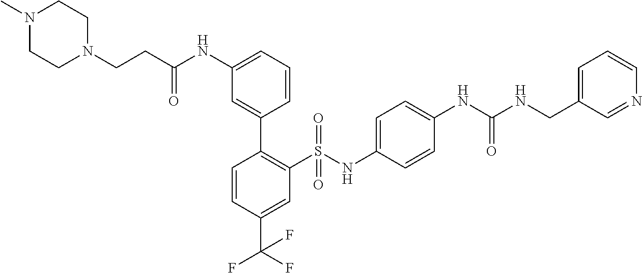 [SMILES: CN1CCN(CCC(=O)Nc2cccc(-c3ccc(C(F)(F)F)cc3S(=O)(=O)Nc3ccc(NC(=O)NCc4cccnc4)cc3)c2)CC1] | 10.19 (s, 1H), 10.09 (s, 1H), 8.73 (s, 1H), 8.61 (s, 1H), 8.58 (d, 1H), 8.24 (s, 1H), 8.01 (d, 1H), 7.96 (d, 1H), 7.64 (d, 1H), 7.62-7.56 (m, 2H), 7.53 (d, 1H), 7.34 (t, 1H), 7.26 (d, 2H), 6.93-6.84 (m, 3H), 6.81 (t, 1H), 4.34 (d, 2H), 3.34-2.31 (m, 15H) |
| 34 | 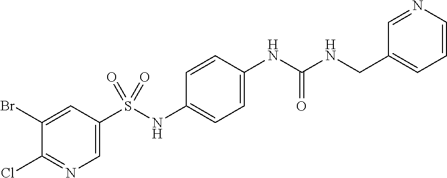 [SMILES: O=C(NCc1cccnc1)Nc1ccc(NS(=O)(=O)c2cnc(Cl)c(Br)c2)cc1] | 10.23 (s, 1H), 8.67 (s, 1H), 8.56 (d, 1H), 8.50 (d, 1H), 8.44 (d, 1H), 8.36 (d, 1H), 7.68 (dt, 1H), 7.37-7.30 (m, 3H), 6.94 (d, 2H), 6.69 (t, 1H), 4.30 (d, 2H) |
| 35 | 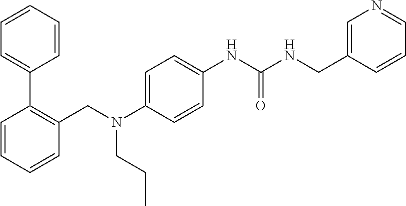 [SMILES: CCCN(Cc1ccccc1-c1ccccc1)c1ccc(NC(=O)NCc2cccnc2)cc1] | 8.48 (br d, 1H), 8.43 (dd, 1H), 8.12 (s, 1H), 7.67 (dt, 1H), 7.50-7.46 (m, 2H), 7.43- 7.39 (m, 3H), 7.34 (ddd, 1H), 7.30-7.27 (m, 2H), 7.23-7.18 (m, 2H), 7.06 (d, 2H), 6.48 (t, 1H), 6.40 (d, 2H), 4.34 (s, 2H), 4.26 (d, 2H), 3.23 (t, 2H), 1.45 (q, 2H), 0. |
| 36 | 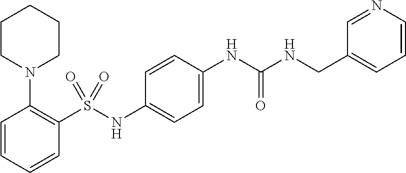 [SMILES: O=C(NCc1cccnc1)Nc1ccc(NS(=O)(=O)c2ccccc2N2CCCCC2)cc1] | 9.96 (s, 1H), 8.59 (s, 1H), 8.49 (s, 1H), 8.43 (br s, 1H), 7.66 (d, 1H), 7.47 (d, 1H), 7.33 (t, 1H), 7.28 (t, 1H), 7.22 (d, 2H), 6.87 (d, 2H), 6.73 (d, 1H), 6.68 (t, 1H), 6.53 (d, 1H), 5.72 (d, 1H), 4.27 (d, 2H), 2.65 (br s, 1H), 1.83-1.03 (m, 10H). |
| 37 | 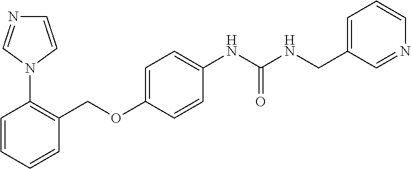 [SMILES: O=C(NCc1cccnc1)Nc1ccc(OCc2ccccc2-n2ccnc2)cc1] | 8.51 (s, 1H), 7.87 (s, 1H), 7.77 (d, 1H), 7.72-7.60 (m, 4H), 7.28 (d, 2H), 6.78 (d, 2H), 6.66 (t, 1H), 4.98 (s, 2H), 4.34 (d, 2H) |
| 38 | 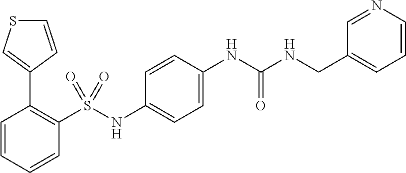 [SMILES: O=C(NCc1cccnc1)Nc1ccc(NS(=O)(=O)c2ccccc2-c2ccsc2)cc1] | NA |
| 39 | 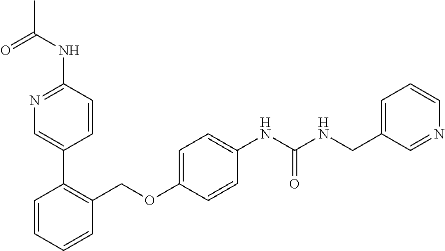 [SMILES: CC(=O)Nc1ccc(-c2ccccc2COc2ccc(NC(=O)NCc3cccnc3)cc2)cn1] | 10.59 (s, 1 H) 8.67-8.76 (m, 2 H) 8.60 (s, 1 H) 8.34 (d, 1 H) 8.21 (d, 1 H) 8.09 (d, 1 H) 7.78-7.87 (m, 2 H) 7.59- 7.65 (m, 1 H) 7.42-7.50 (m, 2 H) 7.36-7.40 (m, 1 H) 7.24- 7.29 (m, 2 H) 6.78-6.84 (m, 2 H) 6.75 (t, 1 H) 4.86 (s, 2 H) 4.40 (d, |
| 40 | 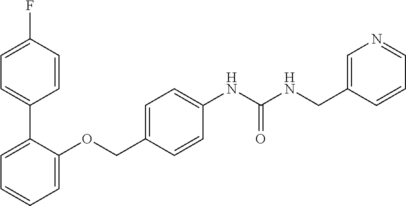 [SMILES: O=C(NCc1cccnc1)Nc1ccc(COc2ccccc2-c2ccc(F)cc2)cc1] | 8.82 (br s, 1H), 8.14 (d, 1H), 7.77-7.74 (m, 1H), 7.57-7.52 (m, 2H), 7.38 (d, 2H), 7.34- 7.28 (m, 2H), 7.25-7.18 (m, 4H), 7.03 (t, 1H), 6.85 (t, 1H), 5.02 (s, 2H), 4.40 (d, 2H) |
| 41 | 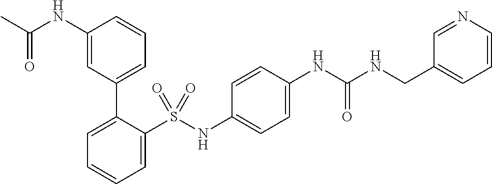 [SMILES: CC(=O)Nc1cccc(-c2ccccc2S(=O)(=O)Nc2ccc(NC(=O)NCc3cccnc3)cc2)c1] | 10.03 (s, 1H), 9.73 (s, 1H), 8.69-8.56 (m, 3H), 7.95-7.90 (m, 2H), 7.63-7.52 (m, 5H), 7.31-7.20 (m, 4H), 6.89-6.85 (m, 3H), 6.75 (t, 1H), 4.33 (d, 2H), 2.05 (s, 3H) |
| 42 | 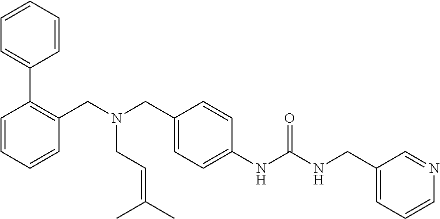 [SMILES: CC(C)=CCN(Cc1ccc(NC(=O)NCc2cccnc2)cc1)Cc1ccccc1-c1ccccc1] | 8.56 (s, 1H), 8.51 (d, 1H), 8.45 (dd, 1H), 7.69 (dt, 1H), 7.66 (d, 1H), 7.46-7.26 (m, 11H), 7.18 (dd, 1H), 7.06 (d, 2H), 6.66 (t, 1H), 5.05 (t, 1H), 4.31 (d, 2H), 3.43 (s, 2H), 3.31 (s, 2H), 2.76 (d, 2H), 1.61 (s, 3H), 1.42 (s, 3H) |
| 43 | 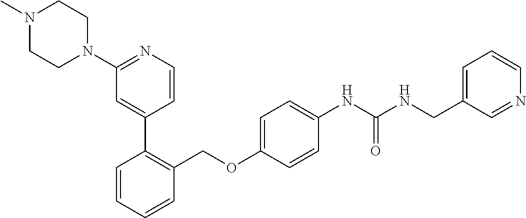 [SMILES: CN1CCN(c2cc(-c3ccccc3COc3ccc(NC(=O)NCc4cccnc4)cc3)ccn2)CC1] | 8.67 (t, 3 H) 8.18 (d, 1 H) 8.13 (d, 1 H) 7.73 (dd, 1 H) 7.60-7.65 (m, 1 H) 7.46- 7.51 (m, 2 H) 7.35-7.41 (m, 1 H) 7.24-7.30 (m, 2 H) 6.96 (s, 1 H) 6.88 (t, 1 H) 6.78- 6.85 (m, 3 H) 4.90 (s, 2 H) 4.30-4.41 (m, 4 H) 3.40 (s, 2 H) 3.03 (br. s., 4 |
| 44 | 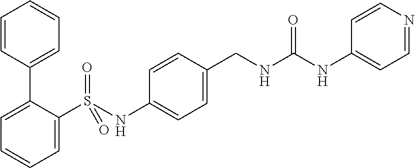 [SMILES: O=C(NCc1ccc(NS(=O)(=O)c2ccccc2-c2ccccc2)cc1)Nc1ccncc1] | 10.06 (s, 1H), 9.89 (s, 1H), 8.40 (d, 2H), 7.98 (d, 1H), 7.65-7.53 (m, 4H), 7.42-7.36 (m, 3H), 7.29 (d, 1H), 7.26- 7.20 (m, 2H), 7.20-7.11 (m, 3H), 6.90 (d, 2H), 4.21 (d, 2H) |
| 45 | 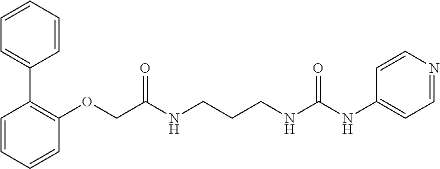 [SMILES: O=C(COc1ccccc1-c1ccccc1)NCCCNC(=O)Nc1ccncc1] | n/a |
| 46 | 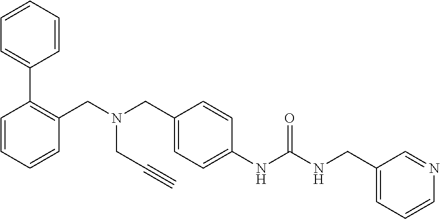 [SMILES: C#CCN(Cc1ccc(NC(=O)NCc2cccnc2)cc1)Cc1ccccc1-c1ccccc1] | 8.60 (s, 1H), 8.52 (d, 1H), 8.45 (dd, 1H), 7.70 (dt, 1H), 7.61 (dd, 1H), 7.49-7.28 (m, 11H), 7.23 (dd 1H), 7.04 (d, 2H), 6.67 (t, 1H), 4.31 (d, 2H), 3.59 (s, 2H), 3.39 (s, 2H), 3.09 (t, 1H), 3.05 (d, 2H) |
| 47 | 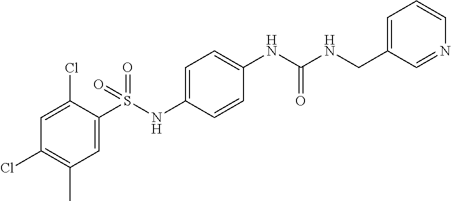 [SMILES: Cc1cc(S(=O)(=O)Nc2ccc(NC(=O)NCc3cccnc3)cc2)c(Cl)cc1Cl] | 10.30 (s, 1H), 8.58 (s, 1H), 8.49 (s, 1H), 8.46-8.41 (m, 1H), 7.92 (s, 1H), 7.80 (s, 1H), 7.69-7.64 (m, 1H), 7.37- 7.30 (m, 1H), 7.25 (d, 2H), 6.96 (d, 2H), 6.67 (t, 1H), 4.27 (d, 2H), 2.32 (s, 3H) |
| 48 | 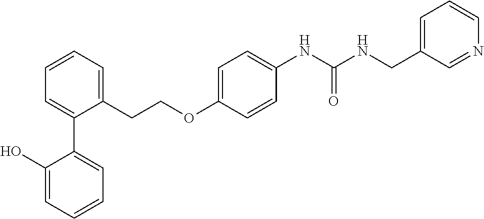 [SMILES: O=C(NCc1cccnc1)Nc1ccc(OCCc2ccccc2-c2ccccc2O)cc1] | 9.43 (s, 1H), 8.53 (d, 1H), 8.47 (dd, 1H), 8.38 (s, 1H), 7.72 (d, 1H), 7.39 (d, 2H), 7.31-7.17 (m, 5H), 7.11-7.06 (m, 2H), 6.94-6.86 (m, 2H), 6.57 (m, 3H)4.29 (d, 2H), 3.91 (m, 2H), 2.86 (m, 2H) |
| 49 | 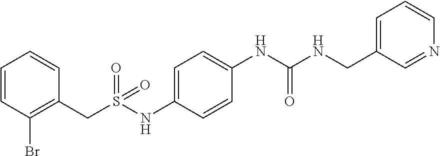 [SMILES: O=C(NCc1cccnc1)Nc1ccc(NS(=O)(=O)Cc2ccccc2Br)cc1] | 9.78 (s, 1H), 8.61 (s, 1H), 8.52 (s, 1H), 8.47-8.44 (m, 1H), 7.72-7.68 (m, 1H), 7.65 (d, 1H), 7.42-7.27 (m, 6H), 7.10 (d, 2H), 6.68 (t, 1H), 4.53 (s, 2H), 4.31 (d, 2H) |
| 50 | 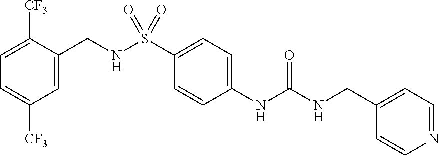 [SMILES: O=C(NCc1ccncc1)Nc1ccc(S(=O)(=O)NCc2cc(C(F)(F)F)ccc2C(F)(F)F)cc1] | 9.15 (br s, 1H), 8.53 (d, 1H), 8.47 (dd, 1H), 8.32 (s, 1H), 7.93-7.89 (m, 2H), 7.82 (br d, 1H), 7.71 (dt, 1H), 7.60 (d, 2H), 7.54 (d, 2H), 7.37 (dd, 1H), 6.93 (t, 1H), 4.33 (d, 2H), 4.23 (br s, 2H) |
| 51 | 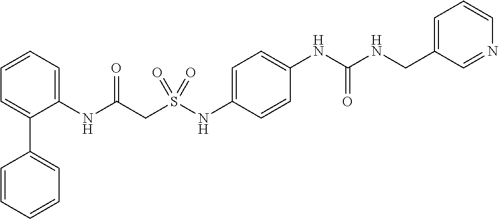 [SMILES: O=C(CS(=O)(=O)Nc1ccc(NC(=O)NCc2cccnc2)cc1)Nc1ccccc1-c1ccccc1] | 9.62 (s, 2H), 8.59 (s, 1H), 8.52 (d, 1H), 8.45 (dd, 1H), 7.70 (m, 1H), 7.64 (d, 1H), 7.42 (s, 2H), 7.41 (d, 2H), 7.39-7.26 (m, 8H), 7.08 (d, 2H), 6.67 (d, 1H), 4.31 (d, 2H), 3.98 (s, 2H) |
| 52 | 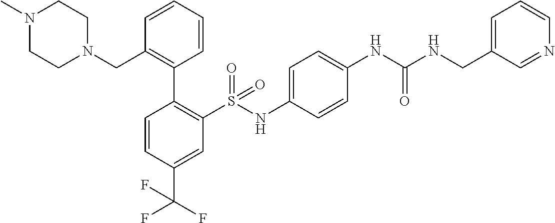 [SMILES: CN1CCN(Cc2ccccc2-c2ccc(C(F)(F)F)cc2S(=O)(=O)Nc2ccc(NC(=O)NCc3cccnc3)cc2)CC1] | 10.04 (s, 1H), 9.64 (s, 1H), 8.77 (s, 1H), 8.52 (s, 1H), 8.46 (d, 1H), 8.30 (s, 1H), 7.98 (d, 1H), 7.71 (d, 1H), 7.51-7.18 (m, 7H), 6.88-6.76 (m, 3H), 6.67 (d, 1H), 4.30 (d, 2H), 3.31-2.97 (m, 5H), 2.90-2.75 (m, 2H), 2.75-2.59 (m, 4H), 2.16-1.98 (m, 2H) |
| 53 | 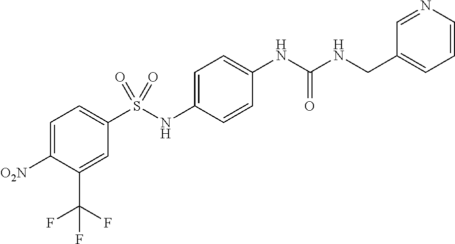 [SMILES: O=C(NCc1cccnc1)Nc1ccc(NS(=O)(=O)c2ccc([N+](=O)[O-])c(C(F)(F)F)c2)cc1] | 10.34 (s, 1H), 8.81 (s, 1H), 8.68 (br s, 1H), 8.33 (d, 1H), 8.18-8.15 (m, 2H), 8.13 (d, 1H), 7.75-7.71 (m, 1H), 7.32 (d, 2H), 6.94 (d, 2H), 6.83 (t, 1H), 4.38 (d, 2H) |
| 54 | 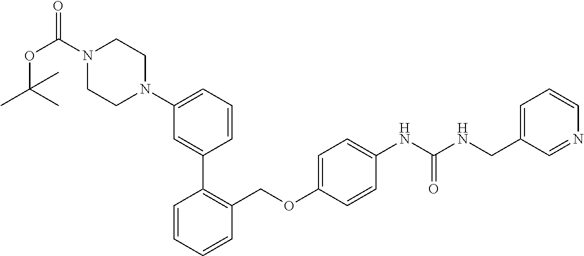 [SMILES: CC(C)(C)OC(=O)N1CCN(c2cccc(-c3ccccc3COc3ccc(NC(=O)NCc4cccnc4)cc3)c2)CC1] | 8.69 (s, 1H), 8.66 (d, 1H), 8.59 (s, 1H), 8.14 (d, 1H), 7.77-7.70 (m, 1H), 7.60-7.55 (m, 1H), 7.47-7.22 (m, 6H), 7.00-6.91 (m, 2H), 6.87-6.77 (m, 3H), 6.73 (t, 1H), 4.84 (s, 2H), 4.38 (d, 2H), 3.39-3.26 (m, 4H), 3.06-2.93 (m, 4H), 1.39 (s, 9H) |
| 55 | 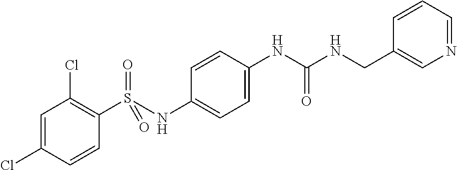 [SMILES: O=C(NCc1cccnc1)Nc1ccc(NS(=O)(=O)c2ccc(Cl)cc2Cl)cc1] | 10.35 (s, 1H), 8.58 (s, 1H), 8.54-8.38 (m, 2H), 7.95-7.79 (m, 2H), 7.67 (d, 1H), 7.56 (d, 1H), 7.39-7.30 (m, 1H), 7.25 (d, 2H), 6.95 (d, 2H), 6.66 (t, 1H), 4.28 (d, 2H) |
| 56 | 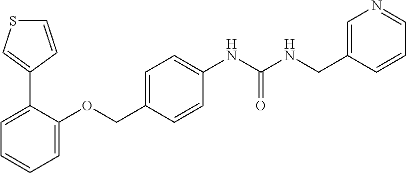 [SMILES: O=C(NCc1cccnc1)Nc1ccc(COc2ccccc2-c2ccsc2)cc1] | 8.74 (s, 1H), 8.57 (d, 1H), 8.50 (dd, 1H), 7.76 (dt, 1H), 7.75 (dd, 1H), 7.56-7.53 (m, 2H), 7.50 (dd, 1H), 7.46-7.44 (m. 1H), 7.41 (d, 2H), 7.31 (d, 1H), 7.30-7.25 (m, 1H), 7.18 (dd, 1H), 6.99 (td, 1H), 6.77 (t, 1H), 5.07 (s, 2H), 4.34 (d, 1H) |
| 57 | 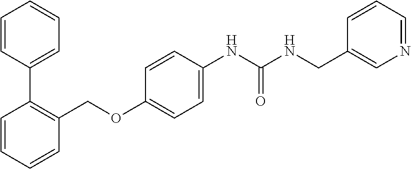 [SMILES: O=C(NCc1cccnc1)Nc1ccc(OCc2ccccc2-c2ccccc2)cc1] | 8.59 (d, 1H), 8.55 (dd, 1H), 8.51 (s, 1H), 7.90 (d, 1H), 7.61-7.51 (m, 2H), 7.46-7.31 (m, 9H), 7.25 (d, 2H), 6.76 (d, 2H), 6.68 (t, 1H), 4.86 (s, 2H), 4.33 (d, 2H) |
| 58 | 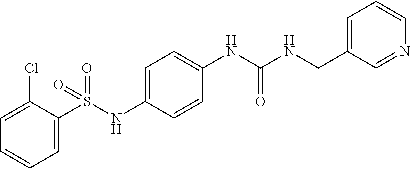 [SMILES: O=C(NCc1cccnc1)Nc1ccc(NS(=O)(=O)c2ccccc2Cl)cc1] | 10.26 (s, 1H), 8.56 (s, 1H), 8.48 (s, 1H), 8.45-8.42 (m, 1H), 7.96-7.90 (m, 1H), 7.70- 7.56 (m, 3H), 7.50-7.43 (m, 1H), 7.37-7.31 (m, 1H), 7.23 (d, 2H), 6.95 (d, 2H), 6.66 (t, 1H), 4.27 (d, 2H) |
| 59 | 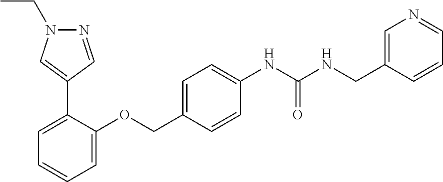 [SMILES: CCn1cc(-c2ccccc2OCc2ccc(NC(=O)NCc3cccnc3)cc2)cn1] | 8.72 (s, 1H), 8.53 (d, 1H), 8.45 (dd, 1H), 8.08 (d, 1H), 7.85 (d, 1H), 7.71 (dt, 1H), 7.58 (dd, 1H), 7.44 (d, 2H), 7.37 (d, 3H), 7.18-7.11 (m, 2H), 6.96-6.92 (m, 1H), 6.73 (t, 1H), 5.09 (s, 2H), 4.32 (d, 2H), 4.10 (q, 2H), 1.35 (t, 3H) |
| 60 | 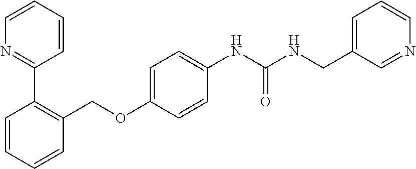 [SMILES: O=C(NCc1cccnc1)Nc1ccc(OCc2ccccc2-c2ccccn2)cc1] | 8.68-8.79 (m, 4 H) 8.37 (d, 1 H) 8.06 (td, 1 H) 7.94 (dd, 1 H) 7.76 (d, 1 H) 7.62-7.67 (m, 1 H) 7.47-7.57 (m, 4 H) 7.20-7.27 (m, 2 H) 6.90- 7.00 (m, 1 H) 6.65-6.72 (m, 2 H) 5.15 (s, 2 H) 4.43 (d, 2 H). |
| 61 | 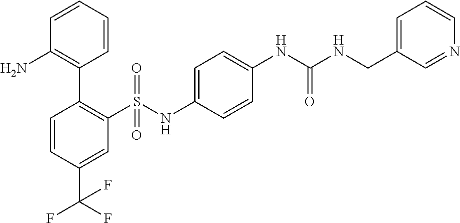 [SMILES: Nc1ccccc1-c1ccc(C(F)(F)F)cc1S(=O)(=O)Nc1ccc(NC(=O)NCc2cccnc2)cc1] | 8.76 (s, 1H), 8.72-8.65 (m, 2H), 8.22-8.15 (m, 2H), 8.03- 7.99 (m, 1H), 7.81-7.75 (m, 1H), 7.50 (d, 1H), 7.44-7.11 (m, 1H), 6.93-6.87 (m, 2H), 6.84-6.79 (m, 2H), 6.71-6.65 (m, 2H), 4.39 (d, 2H) |
| 62 | 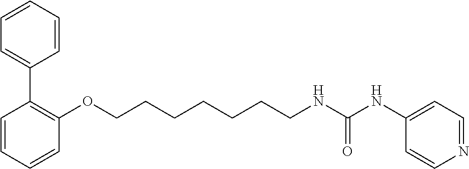 [SMILES: O=C(NCCCCCCCOc1ccccc1-c1ccccc1)Nc1ccncc1] | d 10.30 (s, 1H), 8.50 (d, 2H), 7.81 (d, 2H), 7.48 (dt, 2H), 7.38 (tt, 2H), 7.29 (m, 2H), 7.12 (t, 1H), 7.09 (d, 1H), 7.01 (dt, 1H), 6.45 (bs, 1H), 3.97 (t, 2H), 3.11 (q, 2H), 1.64 (p, 2H), 1.43 (p, 2H), 1.24-1.38 (m, 6H) |
| 63 | 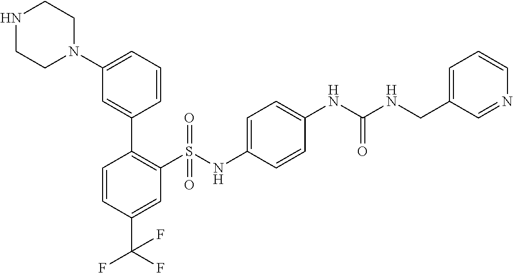 [SMILES: O=C(NCc1cccnc1)Nc1ccc(NS(=O)(=O)c2cc(C(F)(F)F)ccc2-c2cccc(N3CCNCC3)c2)cc1] | 10.08 (s, 1H), 8.81-8.74 (m, 2H), 8.40-8.33 (m, 1H), 8.28 (s, 1H), 8.01-7.90 (m, 2H), 7.55 (d, 1H), 7.32-7.22 (m, 4H), 7.09-7.02 (m, 2H), 6.88- 6.81 (m, 3H), 6.75 (d, 1H), 4.43 (d, 2H), 3.43-3.34 (m, 4H), 3.28-3.16 (m, 4H) |
| 64 | 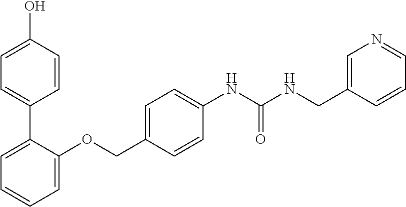 [SMILES: O=C(NCc1cccnc1)Nc1ccc(COc2ccccc2-c2ccc(O)cc2)cc1] | 9.42 (s, 1H), 8.67 (s, 1H), 8.52 (d, 1H), 8.45 (dd, 1H), 7.70 (dt, 1H), 7.39-7.31 (m, 5H), 7.27-7.22 (m, 4H), 7.13 (d, 1H), 6.98 (td, 1H), 6.76 (d, 2H), 6.73 (t, 1H), 4.99 (s, 2H), 4.31 (d, 2H) |
| 65 | 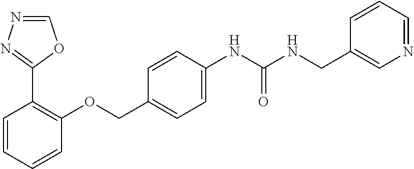 [SMILES: O=C(NCc1cccnc1)Nc1ccc(COc2ccccc2-c2nnco2)cc1] | 9.35 (s, 1H), 8.72 (s, 1H), 8.56-8.53 (m, 1H), 8.50-8.46 (m, 1H), 7.88-7.85 (m, 1H), 7.79-7.74 (m, 1H), 7.63-7.55 (m, 1H), 7.45-7.32 (m, 6H), 7.16-7.11 (m, 1H), 6.75 (t, 1H), 5.19 (s, 2H), 4.33 (d, 2H) |
| 66 | 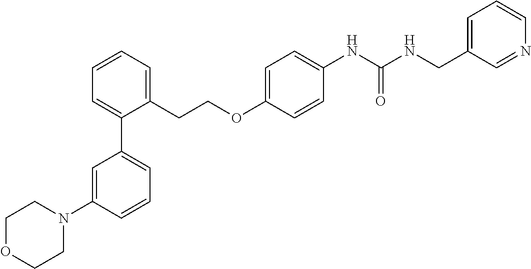 [SMILES: O=C(NCc1cccnc1)Nc1ccc(OCCc2ccccc2-c2cccc(N3CCOCC3)c2)cc1] | 8.54 (d, 1H), 8.48 (dd, 1H), 8.42 (s, 1H), 7.76 (dt, 1H), 7.45-7.40 (m, 2H), 7.35-7.26 (m, 3H), 7.23-7.18 (m, 3H), 6.96 (dd, 1H), 6.88 (t, 1H), 6.77 (dt, 1H), 6.64-6.59 (m, 3H), 4.30 (d, 2H), 3.97 (t, 2H), 3.70 (m, 4H), 3.12 (m, 4H), 2.96 (t, 2H), |
| 67 | 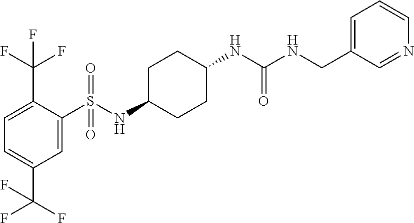 [SMILES: O=C(NCc1cccnc1)N[C@H]1CC[C@H](NS(=O)(=O)c2cc(C(F)(F)F)ccc2C(F)(F)F)CC1] | 8.69-8.60 (m, 2H), 8.43 (s, 1H), 8.35 (d, 1H), 8.27-8.21 (m, 2H), 8.09 (d, 1H), 7.98- 7.90 (m, 1H), 7.78-7.70 (m, 1H), 6.41 (s, 1H), 6.06-5.95 (m, 1H), 4.33-4.22 (m, 2H), 3.31-3.00 (m, 2H), 1.82-1.58 (m, 4H), 1.40-1.23 (m, 2H), 1.18-1.00 (m, 2H) |
| 68 | 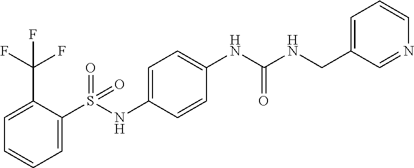 [SMILES: O=C(NCc1cccnc1)Nc1ccc(NS(=O)(=O)c2ccccc2C(F)(F)F)cc1] | 10.29 (s, 1H), 8.60 (s, 1H), 8.55-8.39 (m, 2H), 8.08-7.90 (m, 2H), 7.89-7.74 (m, 2H), 7.72-7.61 (m, 1H), 7.43-7.15 (m, 3H), 7.02-6.88 (m, 2H), 6.72-6.61 (m, 1H), 4.31-4.24 (m, 2H) |
| 69 | 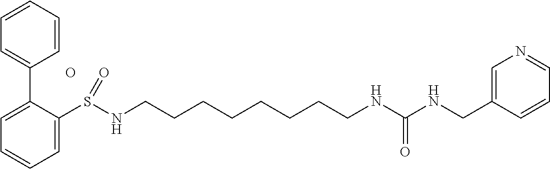 | 8.68 (bs, 1H), 8.66 (bs, 1H), 8.15 (d, 1H), 7.93 (dd, 1H), 7.77 (dd, 1H), 7.65 (dt, 1H), 7.58 (dt, 1H), 7.38 (m, 5H), 7.34 (dt, 1H), 7.20 (t, 1H), 6.51 (s, 1H), 6.14 (s, 1H), 4.30 (d, 1H), 2.96 (t, 2H), 2.63 (q, 2H), 1.32 (p, 4H), 1.17 (m, 8H) |
| 70 | 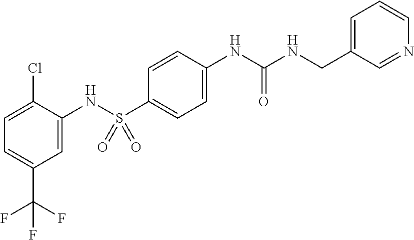 [SMILES: O=C(NCc1cccnc1)Nc1ccc(S(=O)(=O)Nc2cc(C(F)(F)F)ccc2Cl)cc1] | 10.22 (s, 1H), 9.35 (s, 1H), 8.67 (s, 1H), 8.61 (s, 1H), 8.04 (s, 1 h), 7.69-7.50 (m, 7H), 7.11 (bs, 1H), 4.39 (d, 2H) |
| 71 | 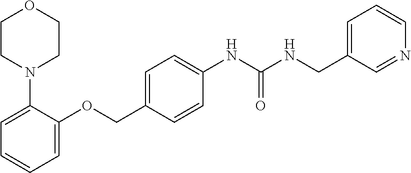 [SMILES: O=C(NCc1cccnc1)Nc1ccc(COc2ccccc2N2CCOCC2)cc1] | 8.92 (s, 1H), 8.79-8.75 (m, 1H), 8.74-8.70 (m, 1H), 8.28 (d, 1H), 7.89-7.83 (m, 1H), 7.43 (d, 2H), 7.33 (d, 2H), 7.05-6.86 (m, 5H), 5.02 (s, 2H), 4.44 (d, 2H), 3.78-3.63 (m4H), 3.08-2.93 (m, 4H) |
| 72 | 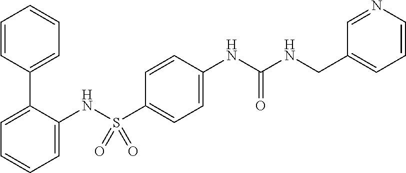 [SMILES: O=C(NCc1cccnc1)Nc1ccc(S(=O)(=O)Nc2ccccc2-c2ccccc2)cc1] | 9.26 (s, 2H), 8.66 (br s, 1H), 8.59 (d, 1H), 8.00 (br d, 1H), 7.62 (dd, 1H), 7.50-7.43 (m, 4H), 7.38-7.33 (m, 3H), 7.27-7.23 (m, 5H), 7.06-7.02 (m, 2H), 4.40 (d, 2H) |
| 73 | 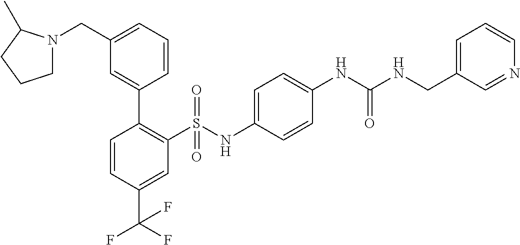 [SMILES: CC1CCCN1Cc1cccc(-c2ccc(C(F)(F)F)cc2S(=O)(=O)Nc2ccc(NC(=O)NCc3cccnc3)cc2)c1] | 10.16 (s, 1H), 9.53 (bs, 1H), 8.83 (s, 1H), 8.65 (s, 1H), 8.62 (d, 1H), 8.30 (s, 1H), 8.07-8.01 (m, 2H), 7.69-7.63 (m, 1H), 7.57 (d, 2H), 7.53- 7.48 (m, 1H), 7.37 (s, 1H), 7.31-7.30 (m, 1H), 7.27 (d, 2H), 6.91 (t, 1H), 6.84 (d, 2H), 4.54 (d, 1H), 4.54 (d, |
| 74 | 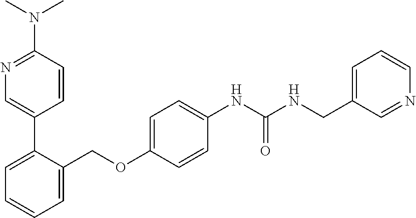 [SMILES: CN(C)c1ccc(-c2ccccc2COc2ccc(NC(=O)NCc3cccnc3)cc2)cn1] | 8.76-8.70 (m, 2H), 8.24 (bd, 1H), 8.06 (d, 2H), 7.96 (dd, 1H), 7.84 (dd, 1H), 7.61 (m, 2H), 7.49-7.45 (m, 2H), 7.38- 7.35 (m, 1H), 7.28 (d, 2H), 7.14 (d, 1H), 6.95 (t, 1H), 6.82 (d, 2H), 4.93 (s, 2H), 4.41 (d, 2H), 3.18 (s, 6H). |
| 75 | 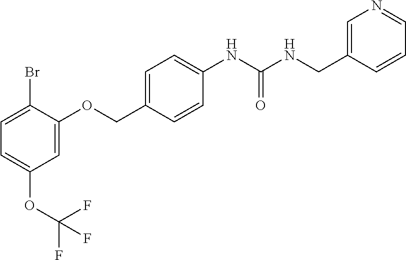 [SMILES: O=C(NCc1cccnc1)Nc1ccc(COc2cc(OC(F)(F)F)ccc2Br)cc1] | 8.71 (s, 1H), 8.52 (d, 1H), 8.45 (dd, 1H), 7.71 (d, 2H), 7.43 (d, 2H), 7.38-7.32 (m, 3H), 7.25 (d, 1H), 6.92 (dd, 1H), 6.73 (t, 1H), 5.14 (s, 2H), 4.32 (d, 2H) |
| 76 | 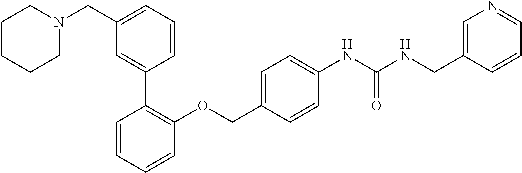 [SMILES: O=C(NCc1cccnc1)Nc1ccc(COc2ccccc2-c2cccc(CN3CCCCC3)c2)cc1] | 8.36 (s, 1H), 8.92 (s, 1H), 8.67 (s, 1H), 8.63 (d, 1H), 8.05 (d, 1H), 1.63 (m, 3H), 7.49 (t, 1H), 7.39 (m, 5H), 7.25 (t, 3H), 7.07 (t, 1H), 6.98 (t, 1H), 5.02 (s, 2H), 4.38 (d, 2H), 4.26 (d, 2H)3.28 (d, 2H), 2.76 (q, 2H), 1.70 (d, 2H), 1.58 (m, 3H), 1.31 |
| 77 | 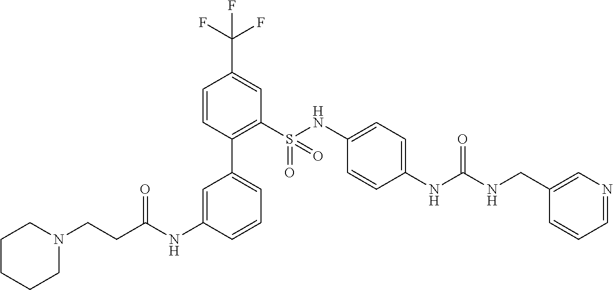 [SMILES: O=C(CCN1CCCCC1)Nc1cccc(-c2ccc(C(F)(F)F)cc2S(=O)(=O)Nc2ccc(NC(=O)NCc3cccnc3)cc2)c1] | 10.33 (s, 1H), 10.10 (s, 1H), 9.10 (bs, 1H), 8.80 (s, 1H), 8.71-8.63 (m, 2H), 8.24 (s, 1H), 8.12 (d, 1H), 8.04-7.99 (m, 1H), 7.75-7.69 (m, 1H), 7.66-7.57 (m, 2H), 7.54 (d, 1H), 7.36 (t, 1H), 7.26 (d, 2H), 6.94-6.85 (m, 4H), 4.38 (d, 2H), 3.49-3.40 (m, 2H |
| 78 | 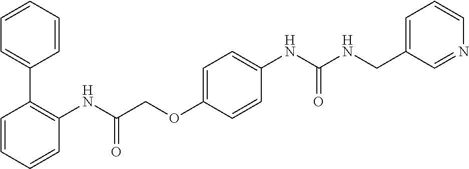 [SMILES: O=C(COc1ccc(NC(=O)NCc2cccnc2)cc1)Nc1ccccc1-c1ccccc1] | 9.14 (s, 1H), 8.53 (s, 1H), 8.47 (s, 1H), 8.46 (d, 1H), 7.96 (d, 1H), 7.73 (d, 1H), 7.53-7.25 (m, 12H), 6.67 (d, 2H), 6.64 (t, 1H), 4.49 (s, 2H), 4.30 (d, 2H) |
| 79 | 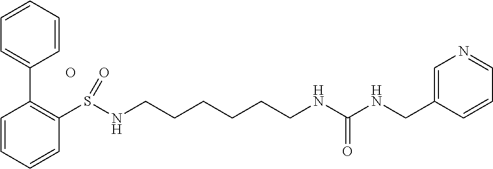 | d 8.68 (dd, 1H), 8.66 (d, 1H), 8.15 (d, 1H), 7.92 (dd, 1H), 7.79 (dd, 1H), 7.65 (dt, 1H), 7.58 (dt, 1H), 7.38 (m, 5H), 7.35 (dt, 1H), 7.21 (t, 1H), 6.49 (bs, 1H), 6.13 (bs, 1H), 4.31 (d, 2H), 2.94 (bs, 2H), 2.63 (q, 2H), 1.30 (m, 4H), 1.15 (m, 4H) |
| 80 | 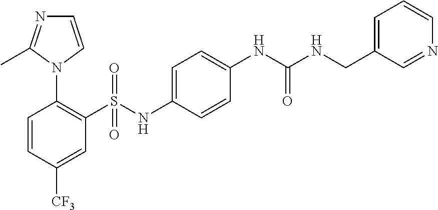 [SMILES: Cc1nccn1-c1ccc(C(F)(F)F)cc1S(=O)(=O)Nc1ccc(NC(=O)NCc2cccnc2)cc1] | 10.65 (s, 1H), 8.83 (s, 1H), 8.39 (d, 1H), 8.35 (s, 1H), 8.05 (d, 2H), 7.97 (bs, 1H), 7.84-7.58 (m, 3H), 7.33 (d, 2H), 6.92 (d, 2H), 6.86 (q, 1H), 4.38 (d, 2H), 2.21 (s, 3H) |
| 81 | 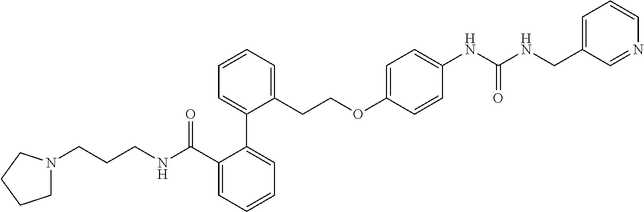 [SMILES: O=C(NCc1cccnc1)Nc1ccc(OCCc2ccccc2-c2ccccc2C(=O)NCCCN2CCCC2)cc1] | 8.65 (d, 1H), 8.62 (dd, 1H), 8.57 (s, 1H), 8.28 (t, 1H), 8.04 (dt, 1H), 7.67 (dd, 1H), 7.53-7.47 (m, 3H), 7.42 (dd, 1H), 7.35-7.23 (m, 3H), 7.20 (d, 2H), 7.15 (dd, 1H), 6.77 (t, 1H), 6.61 (d, 2H), 4.36 (d, 2H), 4.02-3.88 (m, 2H), 3.44- 3.35 (m, 2H), 3.18 |
| 82 | 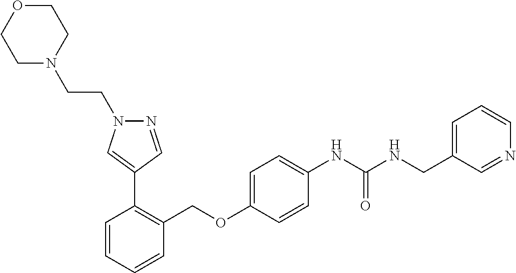 [SMILES: O=C(NCc1cccnc1)Nc1ccc(OCc2ccccc2-c2cnn(CCN3CCOCC3)c2)cc1] | 8.78 (s, 1H), 8.75-8.68 (m, 2H), 8.24-8.20 (bd, 1H), 8.06 (s, 1H), 7.81 (dd, 1H), 7.75 (bs, 1H), 7.56 (dd, 1H), 7.47 (dd, 1H), 7.41 (td, 1H), 7.35- 7.30 (m, 3H), 6.98 (t, 1H), 6.90 (d, 2H), 4.99 (s, 2H), 4.57 (t, 2H), 4.42 (d, 2H), 3.93-3.69 (m, 4H), 3.62 |
| 83 | 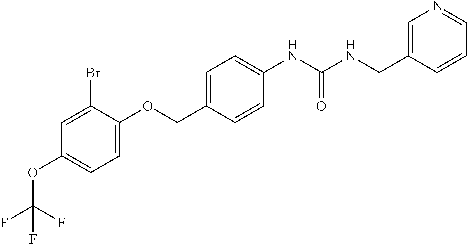 [SMILES: O=C(NCc1cccnc1)Nc1ccc(COc2ccc(OC(F)(F)F)cc2Br)cc1] | 8.71 (s, 1H), 8.52 (d, 1H), 8.45 (dd, 1H), 7.71 (dt, 1H), 7.69 (dd, 1H), 7.44-7.27 (m, 7H), 6.73 (t, 1H), 5.13 (s, 2H), 4.32 (d, 2H) |
| 84 | 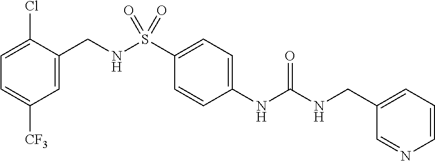 [SMILES: O=C(NCc1cccnc1)Nc1ccc(S(=O)(=O)NCc2cc(C(F)(F)F)ccc2Cl)cc1] | 9.14 (br s, 1H), 8.54 (d, 1H), 8.46 (dd, 1H), 8.19 (s, 1H), 7.23-7.54 (m, 8H), 7.36 (dd, 1H), 6.91 (t, 1H), 4.34 (d, 2H), 4.13 (s, 2H) |
| 85 | 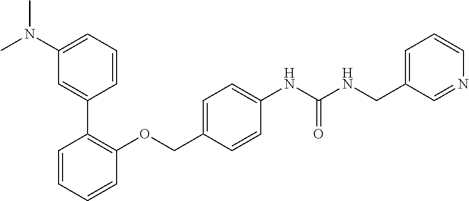 [SMILES: CN(C)c1cccc(-c2ccccc2OCc2ccc(NC(=O)NCc3cccnc3)cc2)c1] | 8.91 (s, 1H), 8.76-8.69 (m, 2H), 8.24 (d, 1H), 7.85-7.80 (m, 1H), 7.38 (d, 2H), 7.34- 7.22 (m, 6H), 7.18 (d, 1H), 7.05-6.98 (m, 2H), 6.80 (d, 1H), 5.00 (s, 2H), 4.43 (d, 2H), 2.90 (s, 6H) |
| 86 | 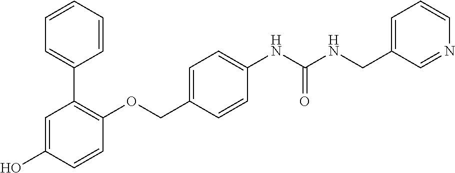 [SMILES: O=C(NCc1cccnc1)Nc1ccc(COc2ccc(O)cc2-c2ccccc2)cc1] | 9.13 (bs, 1H), 8.69 (s, 1H), 8.60 (d, 1H), 8.53 (d, 1H), 7.86-7.78 (m, 8 1H), 7.51 (d, 2H), 7.49-7.33 (m, 4H) , 7.25- 7.14 (m, 2H), 7.09-6.94 (m, 2H), 6.75 (d, 2H), 4.89 (s, 2H), 4.37 (d, 2H) |
| 87 | 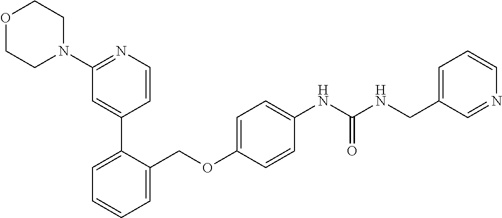 [SMILES: O=C(NCc1cccnc1)Nc1ccc(OCc2ccccc2-c2ccnc(N3CCOCC3)c2)cc1] | 8.70 (s, 1H), 8.68-8.63 (m, 2H), 8.17-8.13 (m, 2H), 7.75 (dd, 1H), 7.63 (dd, 1H), 7.51- 7.48 (m, 2H), 7.40 (dd, 1H), 7.28 (d, 2H), 6.94 (s, 1H), 6.83-6.79 (m, 4H), 4.90 (s, 2H), 4.40 (d, 2H), 3.62-3.57 (m, 4H), 3.41-3.36 (m, 4H). |
| 88 | 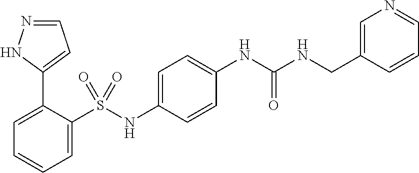 [SMILES: O=C(NCc1cccnc1)Nc1ccc(NS(=O)(=O)c2ccccc2-c2ccn[nH]2)cc1] | 8.77 (d, 1H), 8.69-8.65 (m 2H), 8.15 (dt, 1H), 7.98-7.93 (m, 1H), 7.82-7.63 (m, 3H), 7.54-7.41 (m, 2H), 7.24 (t, 2H), 6.99-6.93 (m, 2H), 6.83 (q, 1H), 6.69 (d, 1H), 4.38- 4.36 (m, 2H) |
| 89 | 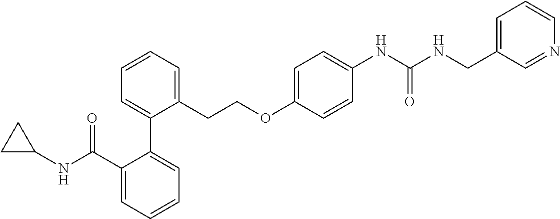 [SMILES: O=C(NCc1cccnc1)Nc1ccc(OCCc2ccccc2-c2ccccc2C(=O)NC2CC2)cc1] | 8.51 (br s, 1H), 8.44 (br d, 1H), 8.38 (s, 1H), 7.94 (d, 1H), 7.69 (dt, 1H), 7.50-7.44 (m, 3H), 7.41-7.23 (m, 4H), 7.22-7.17 (m, 3H), 7.11 (dd, 1H), 6.62-6.56 (m, 3H), 4.29 (d, 2H), 4.01-3.96 (m, 1H), 3.92-3.87 (m, 1H), 2.85-2.79 (m, 2H) |
| 90 | 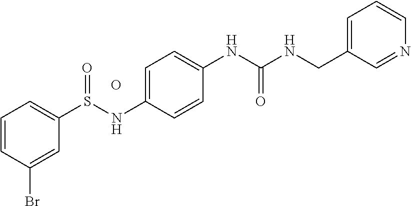 | 10.04 (s, 1H), 8.61 (s, 1H), 8.50 (d, 1H), 8.46-8.43 (m, 1H), 7.85-7.80 (m, 2H), 7.71- 7.62 (m, 2H), 7.52-7.46 (m, 1H), 7.37-7.32 (m, 1H), 7.28 (d, 2H), 6.92 (d, 2H), 6.67 (t, 1H), 4.29 (d, 2H), ) |
| 91 | 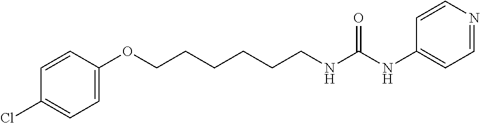 [SMILES: O=C(NCCCCCCOc1ccc(Cl)cc1)Nc1ccncc1] | 8.40 (d, 2H), 7.61 (bs, 2H), 7.30 (d, 2H), 6.95 (d, 2H), 3.95 (t, 2H), 1.71 (t, 2H), 1.50-1.30 (m, 8H) |
| 92 | 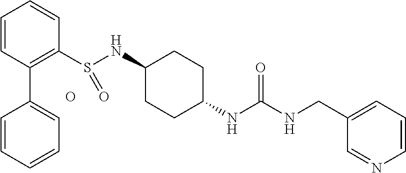 | 8.68 (d, 1H), 8.64 (s, 1H), 8.14 (d, 1H), 7.99 (dd, 1H), 7.64 (dt, 1H), 7.58 (dt, 1H), 7.39 (m, 5H), 7.32 (dd, 1H), 7.25 (d, 1H), 6.41 (s, 1H), 6.00 (d, 1H), 4.29 (d, 2H), 3.17 (m, 1H), 2.67 (m, 1H), 1.64 (dd, 4H), 1.20 (q, 2H), 0.97 (q, 2H) |
| 93 | 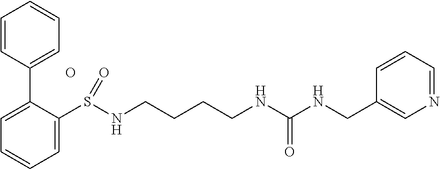 | 8.45 (d, 1H), 8.43 (d, 1H), 7.92 (d, 1H), 7.68-7.56 (m, 3H), 7.42-7.30 (m, 7H), 7.27 (t, 1H), 4.19 (d, 2H), 2.95- 2.87 (m, 2), 2.69-2.61 (m, 2H), 1.33-1.25 (m, 4H) |
| 94 | 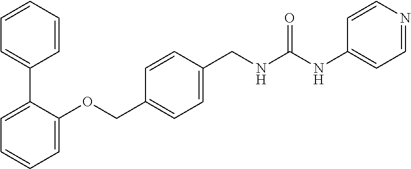 [SMILES: O=C(NCc1ccc(COc2ccccc2-c2ccccc2)cc1)Nc1ccncc1] | 10.36 (s, 1H), 8.51 (d, 2H), 7.80 (d, 2H), 7.62-7.56 (m, 1H), 7.53 (dd, 2H), 7.40 (t, 2H), 7.36-7.26 (m, 7H), 7.18 (d, 1H), 7.04 (t, 1H), 6.57 bs, 1H), 5.11 (s, 2H), 4.33 (d, 2H) |
| 95 | 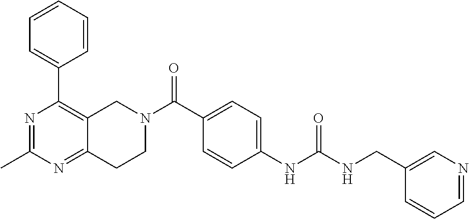 [SMILES: Cc1nc2c(c(-c3ccccc3)n1)CN(C(=O)c1ccc(NC(=O)NCc3cccnc3)cc1)CC2] | 8.91 (s, 1H), 8.53 (s, 1H), 8.48-8.44 (m, 1H), 7.74-7.23 (m, 11H), 6.81 (t, 1H), 4.70 (s, 2H), 4.33 (d, 2H), 3.89-3.72 (m, 2H), 3.05-2.96 (m, 2H), 2.62 (s, 3H) |
| 96 | 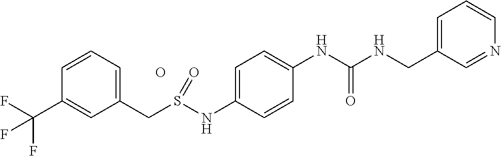 | (400 MHz, MeOH-d4) 8.76 (s, 1H), 8.68 (s, 1H), 8.43 (d, 1H), 7.91 (dd, 1H), 7.64 (d, 1H), 7.58-7.50 (m, 3H), 7.34 (d, 2H), 7.11 (d, 2H), 4.55 (s, 2H), 4.45 (s, 2H) |
| 97 | 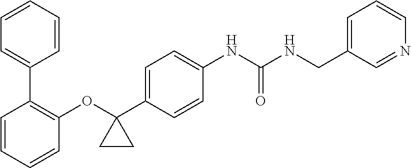 [SMILES: O=C(NCc1cccnc1)Nc1ccc(C2(Oc3ccccc3-c3ccccc3)CC2)cc1] | 8.63 (s, 1H), 8.53 (d, 1H), 8.48 (dd, 1H), 7.53 (d, 2H), 7.47-7.42 (m, 3H), 7.37-7.26 (m, 4H), 7.19-7.14 (m, 1H), 7.08 (d, 2H), 7.00-6.94 (m, 2H), 6.69 (t, 1H), 4.31 (d, 2H), 1.32-1.22 (m, 4H). |
| 98 | 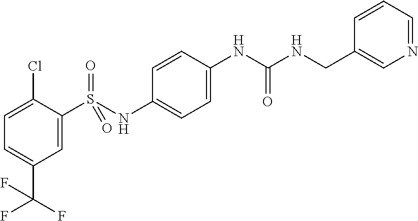 [SMILES: O=C(NCc1cccnc1)Nc1ccc(NS(=O)(=O)c2cc(C(F)(F)F)ccc2Cl)cc1] | 10.54 (s, 1H), 8.80 (s, 1H), 8.72-8.63 (m, 2H), 8.16 (d, 1H), 8.12 (s, 1H), 8.03 (d, 1H), 7.92 (d, 1H), 7.80-7.72 (m, 1H), 7.27 (d, 2H), 6.97 (d, 2H), 6.86 (t, 1H), 4.38 (2H) |
| 99 | 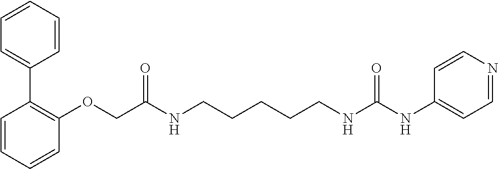 [SMILES: O=C(COc1ccccc1-c1ccccc1)NCCCCCNC(=O)Nc1ccncc1] | 10.10 (s, 1H), 8.49 (d, 2H), 7.78 (d, 2H), 7.63 (t, 1H), 7.59 (d, 2H), 7.43 (t, 2H), 7.38-7.30 (m, 3H), 7.07 (t, 1H), 7.03-6.93 (m, 2H), 6.57 (s, 3H), 4.48 (s, 2H), 3.15- 3.06 (m, 4H), 1.50-1.35 (m, 4H), 1.28-1.20 (m, 2H) |
| 100 | 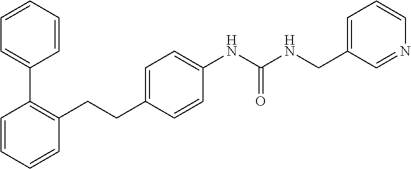 [SMILES: O=C(NCc1cccnc1)Nc1ccc(CCc2ccccc2-c2ccccc2)cc1] | 8.52 (s, 1H), 8.51 (d, 1H), 8.45 (dd, 1H), 7.69 (dt, 1H), 7.49-7.13 (m, 14H), 6.82 (d, 2H), 6.68 (t, 1H), 4.30 (d, 2H), 2.79-2.27 (m, 2H), 2.61- 2.55 (m, 2H) |
| 101 | 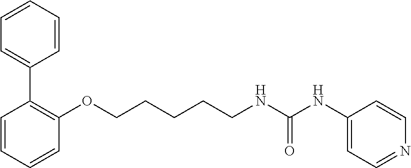 [SMILES: O=C(NCCCCCOc1ccccc1-c1ccccc1)Nc1ccncc1] | 10.75 (s, 1H), 8.52 (d, 2H), 7.85 (bs, 2H), 7.49 (d, 2H), 7.39 (t, 2H), 7.34-7.20 (m, 4H), 7.10 (d, 1H), 7.01 (td, 1H), 3.98 (t, 2H), 1.71-1.63 (m, 2H), 1.49-1.43 (m, 2H), 1.42-1.30 (m, 2H), 1.30-1.21 (m, 4H) |
| 102 | 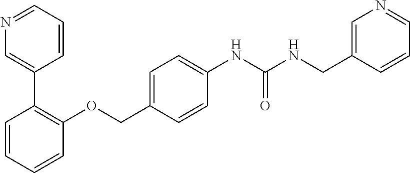 [SMILES: O=C(NCc1cccnc1)Nc1ccc(COc2ccccc2-c2cccnc2)cc1] | 8.70 (dd, 1H), 8.67 (s, 1H), 8.52 (d, 1H), 8.48 (dd, 1H), 8.45 (dd, 1H), 7.91 (dt, 1H), 7.70 (dt, 1H), 7.43-7.34 (m, 6H), 7.23 (d, 2H), 7.07 (td, 1H), 6.72 (t, 1H), 5.04 (s, 2H), 4.31 (d, 2H) |
| 103 | 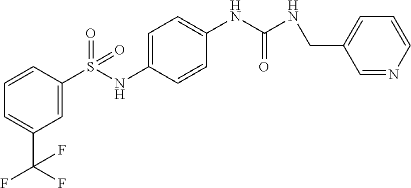 [SMILES: O=C(NCc1cccnc1)Nc1ccc(NS(=O)(=O)c2cccc(C(F)(F)F)c2)cc1] | 10.16-10.03 (m, 1H), 8.68- 8.39 (m, 2H), 8.11-7.60 (m, 6H), 7.41-7.20 (m, 3H), 7.00- 6.85 (m, 2H), 6.73-6.61 (m, 1H), 4.35-4.22 (m, 2H) |
| 104 | 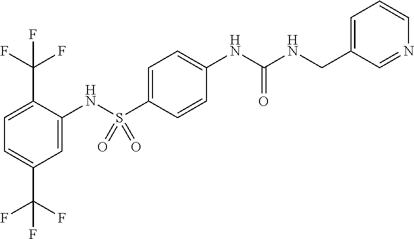 [SMILES: O=C(NCc1cccnc1)Nc1ccc(S(=O)(=O)Nc2cc(C(F)(F)F)ccc2C(F)(F)F)cc1] | (400 MHz, MeOH-d4) 8.57 (bs, 1H), 8.48 (bs, 1H), 7.93 (d, 1H), 7.83 (d, 1H), 7.79 (s, 1H), 7.65 (m, 3H), 7.55 (d, 2H), 7.51 (m, 1H), 4.46 (s, 2H) |
| 105 | 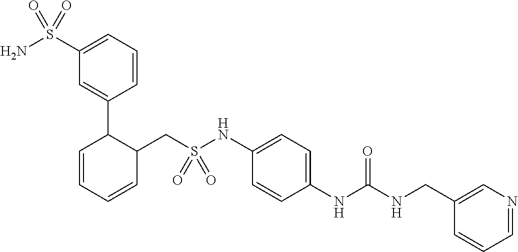 [SMILES: NS(=O)(=O)c1cccc(C2C=CC=CC2CS(=O)(=O)Nc2ccc(NC(=O)NCc3cccnc3)cc2)c1] | |
| 106 | 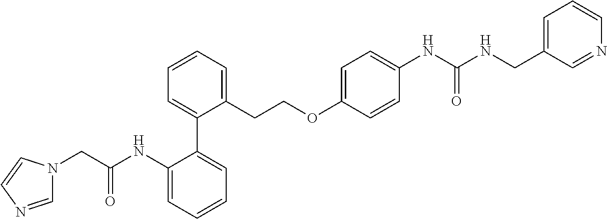 [SMILES: O=C(Cn1ccnc1)Nc1ccccc1-c1ccccc1CCOc1ccc(NC(=O)NCc2cccnc2)cc1] | 9.49 (s, 1H), 8.99 (s, 1H), 8.62 (d, 1H), 8.58 (dd, 1H), 8.52 (s, 1H), 7.97 (dt, 1H), 7.67 (d, 1H), 7.64 (t, 1H), 7.60 (dd, 1H), 7.53 (t, 1H), 7.48 (d, 1H), 7.44-7.26 (m, 5H), 7.19 (d, 2H), 7.14 (d, 1H), 6.73 (t, 1H), 6.59 (d, 2H), 4.97 (s, 2H), 4.35 (d |
| 107 | 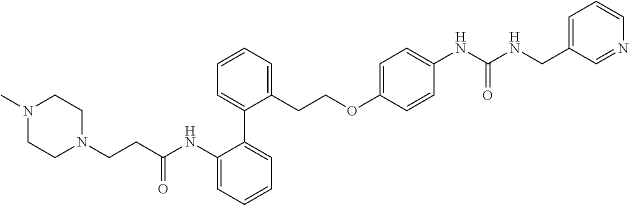 [SMILES: CN1CCN(CCC(=O)Nc2ccccc2-c2ccccc2CCOc2ccc(NC(=O)NCc3cccnc3)cc2)CC1] | 9.14 (br s, 1H), 8.50 (d, 1H), 8.45 (dd, 1H), 8042 (s, 1H), 7.68 (td, 2H), 7.47 (d, 1H), 7.41-7.29 (m, 4H), 7.27-7.19 (m, 4H), 7.15 (d, 1H), 6.62- 6.58 (m, 3H), 4.29 (d, 2H), 3.91-3.84 (m, 2H), 2.87-2.80 (m, 1H), 2.76-2.67 (m, 1H), 2.41-2.20 (m, 8H) |
| 108 | 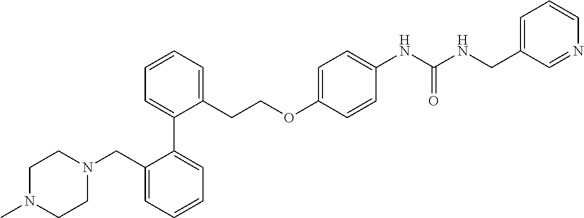 [SMILES: CN1CCN(Cc2ccccc2-c2ccccc2CCOc2ccc(NC(=O)NCc3cccnc3)cc2)CC1] | 8.51 (d, 1H), 8.44 (dd, 1H), 8.39 (s, 1H), 7.69 (dt, 1H), 7.51 (dd, 1H), 7.44 (dd, 1H), 7.40-7.30 (m, 4H), 7.27 (td, 1H), 7.20-7.16 (m, 3H), 7.09 (dd, 1H), 6.59-6.55 (m, 3H), 4.29 (d, 2H), 3.91 (t, 2H), 3.11 (q, 2H), 2.84-2.76 (m, 1H), 2.68-2.61 (m, 1H) |
| 109 | 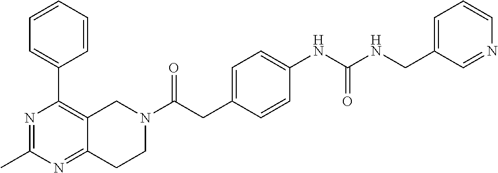 [SMILES: Cc1nc2c(c(-c3ccccc3)n1)CN(C(=O)Cc1ccc(NC(=O)NCc3cccnc3)cc1)CC2] | 8.69 (s, 1H), 8.52 (d, 1H), 8.45 (d, 1H), 7.74-7.66 (m, 1H), 7.62-7.44 (m, 4H), 7.42- 7.28 (m, 3H), 7.07 (d, 2H), 6.80-6.70 (m, 2H), 4.64 (s, 2H), 4.31 (d, 2H), 3.89-3.82 (m, 2H), 3.11-3.00 (m, 2H), 2.59 (s, 3H) |
| 110 | 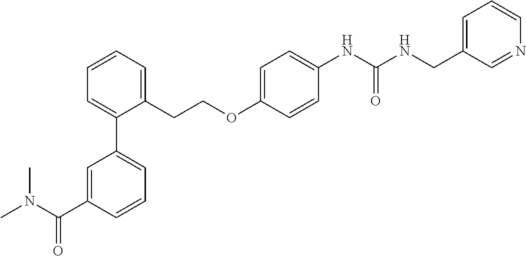 [SMILES: CN(C)C(=O)c1cccc(-c2ccccc2CCOc2ccc(NC(=O)NCc3cccnc3)cc2)c1] | 8.68 (d, 1H), 8.65 (dd, 1H), 8.54 (s, 1H), 8.15 (br d, 1H), 7.74 (dd, 1H), 7.54-7.29 (m, 7H), 7.24-7.21 (m, 3H), 7.17 (br d, 1H), 6.72 (t, 1H), 6.65 (d, 2H), 4.38 (d, 2H), 3.97 (t, 2H), 2.98-2.94 (m, 5H), 2.90 (s, 3H) |
| 111 | 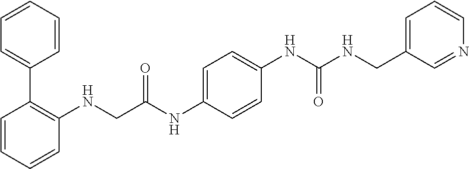 [SMILES: O=C(CNc1ccccc1-c1ccccc1)Nc1ccc(NC(=O)NCc2cccnc2)cc1] | 9.94 (s, 1H), 8.55 (s, 1H), 8.52 (d, 1H), 8.45 (dd, 1H), 7.70 (d, 1H, 7.54-7.27 (m, 11H), 7.19 (t, 1H), 7.03 (dd, 1H), 6.73 (t, 1H), 6.65 (t, 1H), 6.57 (d, 1H), 5.14 (t, 1H, 4.30 (d, 2H), 3.86 (d, 2H) |
| 112 | 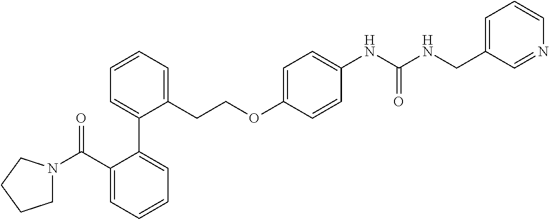 [SMILES: O=C(NCc1cccnc1)Nc1ccc(OCCc2ccccc2-c2ccccc2C(=O)N2CCCC2)cc1] | 8.72 (br s, 1H), 8.67 (br d, 1H), 8.56 (s, 1H), 8.17 (dt, 1H), 7.76 (dd, 1H), 7.47-7.39 (m, 4H), 7.35-7.29 (m, 2H), 7.24-7.19 (m, 3H), 7.09 (dd, 1H), 6.75 (t, 1H), 6.64 (d, 2H), 4.39 (d, 2H), 4.05-3.96 (m, 2H), 3.20-3.15 (m, 1H), 3.13-3.04 (m, 3H), 2.90 |
| 113 | 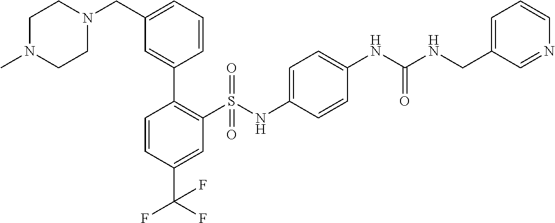 [SMILES: CN1CCN(Cc2cccc(-c3ccc(C(F)(F)F)cc3S(=O)(=O)Nc3ccc(NC(=O)NCc4cccnc4)cc3)c2)CC1] | 10.07 (s, 1H), 8.87 (s, 1H), 8.71-8.66 (m, 2H), 8.30-8.28 (m, 1H), 8.15 (d, 1H), 8.04- 8.00 (m, 1H), 7.78-7.73 (m, 1H), 7.56 (d, 1H), 7.45-7.40 (m, 2H), 7.30-7.24 (m, 3H), 7.23-7.18 (m, 1H), 6.97 (t, 1H), 6.83 (d, 2H), 4.39 (d, 2H), 3.83 (s, 2H), 3.58-2.6 |
| 114 | 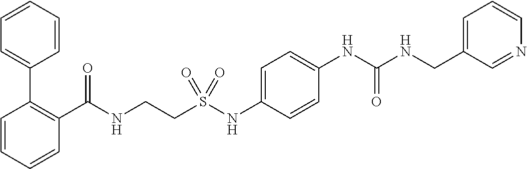 [SMILES: O=C(NCc1cccnc1)Nc1ccc(NS(=O)(=O)CCNC(=O)c2ccccc2-c2ccccc2)cc1] | 9.54 (s, 1H), 8.39 (s, 1H), 8.75 (s, 1H), 8.71 (d, 1H), 8.29-8.21 (m, 2H), 7.82 (dd, 1H), 7.55-7.46 (m, 1H), 7.44- 7.36 (m, 6H), 7.33-7.26 (m, 5H), 4.43 (d, 2H), 3.40 (m, 2H), 2.88 (m, 2H) |
| 115 | 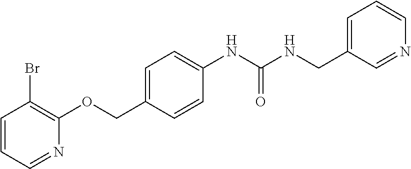 [SMILES: O=C(NCc1cccnc1)Nc1ccc(COc2ncccc2Br)cc1] | 8.68 (s, 1H), 8.51 (d, 1H), 8.44 (dd, 1H), 7.92-7.86 (m, 2H), 7.69 (dt, 1H), 7.38-7.33 (m, 3H), 7.21 (d, 2H), 6.69 (t, 1H), 6.20 (t, 1H), 5.06 (s, 2H), 4.30 (d, 2H) |
| 116 | 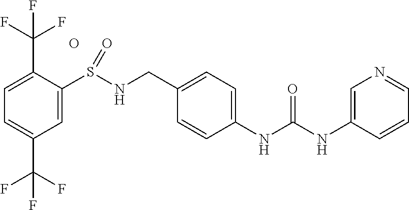 | 8.84 (s, 1H), 8.79 (s, 1H), 8.77 (s, 1H), 8.58 (d, 1H ), 8.18 (dd, 2H), 8.15 (s, 2H), 7.93 (m, 1H), 7.30 (m, 3H), 7.08 (d, 2H), 4.14 (s, 2H) |
| 117 | 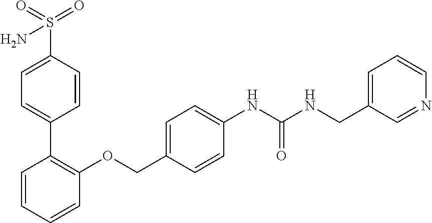 [SMILES: NS(=O)(=O)c1ccc(-c2ccccc2OCc2ccc(NC(=O)NCc3cccnc3)cc2)cc1] | 8.82 (s, 1H), 8.65 (s, 1H), 8.61 (d, 1H), 8.03 (d, 1H), 7.84- 7.79 (m, 2H), 7.67-7.61 (m, 1H), 7.43-7.33 (m, 6H), 7.30- 7.21 (m, 3H), 7.10-7.03 (m, 1H), 6.85 (t, 1H), 5.04 (s, 2H), 4.38 (d, 2H) |
| 118 | 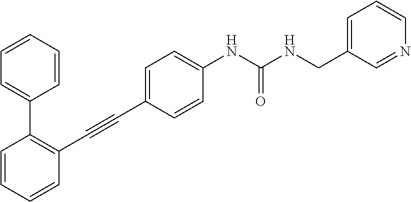 [SMILES: O=C(NCc1cccnc1)Nc1ccc(C#Cc2ccccc2-c2ccccc2)cc1] | 8.87 (s, 1H), 8.50 (d, 2H), 7.71 (d, 1H), 7.67-7.60 (m, 3H), 7.54-7.33 (m, 9H), 7.20 (d, 2H), 6.80 (t, 1H), 4.32 (d, 2H) |
| 119 | 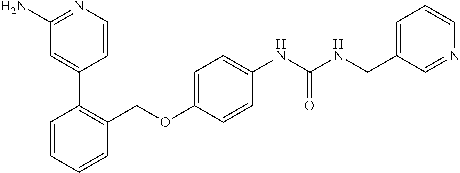 [SMILES: Nc1cc(-c2ccccc2COc2ccc(NC(=O)NCc3cccnc3)cc2)ccn1] | 8.55-8.64 (m, 3 H) 8.06 (br. s., 2 H) 7.91-7.99 (m, 2 H) 7.62-7.70 (m, 1 H) 7.49- 7.58 (m, 3 H) 7.36-7.41 (m, 1 H) 7.25-7.31 (m, 2 H) 6.91- 6.95 (m, 2 H) 6.74-6.82 (m, 3 H) 4.97 (s, 2 H) 4.34 (d, 2 H). |
| 120 | 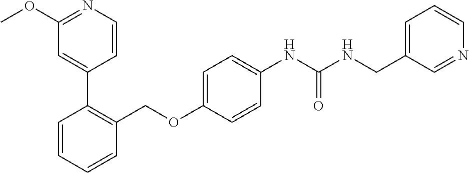 [SMILES: COc1cc(-c2ccccc2COc2ccc(NC(=O)NCc3cccnc3)cc2)ccn1] | 8.72-8.77 (m, 2 H) 8.64 (s, 1 H) 8.30 (d, 1 H) 8.18 (d, 1 H) 7.88 (dd, 1 H) 7.58-7.64 (m, 1 H) 7.45-7.51 (m, 2 H) 7.36 (td, 1 H) 7.24-7.28 (m, 2 H) 7.03-7.06 (m, 1 H) 6.85 (s, 1 H) 6.76-6.81 (m, 3 H) 4.90 (s, 2 H) 4.42 (d, 2 H) 3.83- 3.85 (m, |
| 121 | 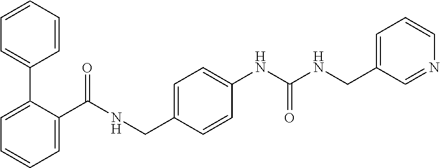 [SMILES: O=C(NCc1cccnc1)Nc1ccc(CNC(=O)c2ccccc2-c2ccccc2)cc1] | 8.79 (s, 1H), 8.74 (s, 1H), 8.71-8.67 (m, 1H), 8.64 (t, 1H), 8.21 (d, 1H), 7.82-7.77 (m, 1H), 7.53-7.46 (m, 1H), 7.45-7.24 (m, 10H), 6.92 (d, 2H), 6.87 (t, 1H), 4.42 (d, 2H), 4.19 (d, 2H) |
| 122 | 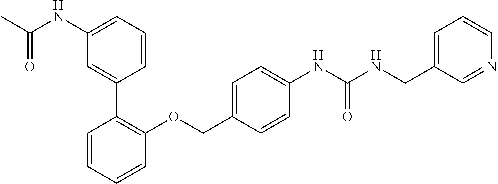 [SMILES: CC(=O)Nc1cccc(-c2ccccc2OCc2ccc(NC(=O)NCc3cccnc3)cc2)c1] | 9.97 (s, 1H), 8.80 (s, 1H), 8.66 (s, 1H), 8.05 (d, 1H), 7.80 (s, 1H), 7.69-7.61 (m, 1H), 7.49 (d, 1H), 7.39-7.22 (m, 7H), 7.16 (d, 2H), 7.02 (t, 1H), 6.84 (t, 1H), 5.02 (s, 2H), 4.38 (d, 2H), 2.06 (s, 3H) |
| 123 | 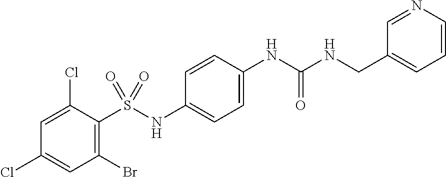 [SMILES: O=C(NCc1cccnc1)Nc1ccc(NS(=O)(=O)c2c(Cl)cc(Cl)cc2Br)cc1] | 10.52 (s, 1H), 8.76 (s, 1H), 8.67 (d, 1H), 8.64 (dd, 1H), 8.11 (dt, 1H), 8.0 (d, 1H), 7.89 (d, 1H), 7.71 (dd, 1H), 7.27 (d, 2H), 6.96 (d, 2H), 6.83 (t, 1H), 4.37 (d, 2H) |
| 124 | 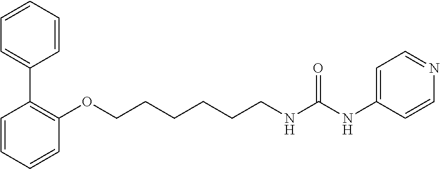 [SMILES: O=C(NCCCCCCOc1ccccc1-c1ccccc1)Nc1ccncc1] | 8.51 (d, 2H), 7.83 (bs, 1H), 7.49 (d, 2H), 7.40 (t, 2H), 7.35-7.19 (m, 3H), 7.10 (d, 1H), 7.02 (t, 1H), 3.97 (t, 2H), 1.68-1.59 (m, 2H), 1.47- 1.41 (m, 2H), 1.40-1.26 (m, 6H) |
| 125 | 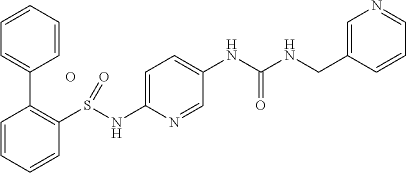 | 8.75 (s, 1H), 8.60 (d, 1H), 8.56 (dd, 1H), 8.12 (s, 1H), 8.05 (dd, 1H), 7.93 (d, 1H), 7.56 (m, 4H), 7.34 (m, 3H), 7.27 (m, 3H), 6.91 (t, 1H), 6.79 (d, 1H), 4.34 (d, 2H) |
| 126 | 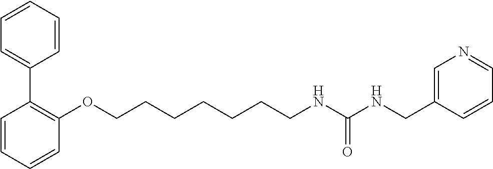 [SMILES: O=C(NCCCCCCCOc1ccccc1-c1ccccc1)NCc1cccnc1] | 8.45 (d, 1H), 8.41 (dd, 1H), 7.65 (dt, 1H), 7.48 (m, 2H), 7.39 (m, 2H), 7.31 (m, 4H), 7.08 (d, 1H), 7.01 (dt, 1H), 6.36 (t, 1H), 5.97 (t, 1H), 2.25 (d, 2H), 3.96 (t, 2H), 1.6 (p, 2H), 1.29 (m, 10H) |
| 127 | 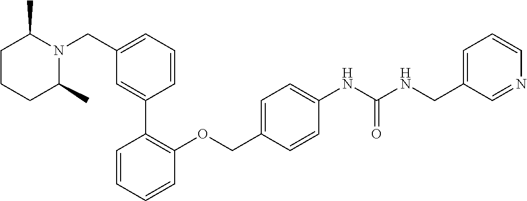 [SMILES: C[C@@H]1CCC[C@H](C)N1Cc1cccc(-c2ccccc2OCc2ccc(NC(=O)NCc3cccnc3)cc2)c1] | 9.17-9.03 (m, 1H), 8.91 (s, 1H), 8.68 (s, 1H), 8.64 (d, 1H), 8.09 (d, 1H), 7.74-7.42 (m, 5H), 7.40-7.31 (m, 4H), 7.26-7.17 (m, 3H), 7.10-7.04 (m, 1H), 6.98 (t, 1H), 5.03 (s, 2H), 4.51 (s, 1H), 4.39 (d, 2H), 4.31 (d, 1H), 3.47 (bs, 1H), 3.03 (bs, 1H), 1.8 |
| 128 | 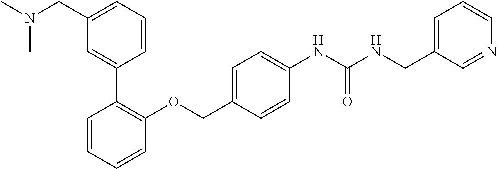 [SMILES: CN(C)Cc1cccc(-c2ccccc2OCc2ccc(NC(=O)NCc3cccnc3)cc2)c1] | 8.91 (s, 1H), 8.66 (d, 1H), 8.62 (d, 1H), 8.04 (d, 1H), 7.70-7.59 (m, 3H), 7.49 (t, 1H), 7.45-7.32 (m, 5H), 7.28- 7.20 (m, 3H), 7.09-7.04 (m, 1H), 7.02-6.96 (m, 1H), 5.02 (s, 2H), 4.38 (d, 2H), 4.28 (s, 2H), 2.68 (s, 6H) |
| 129 | 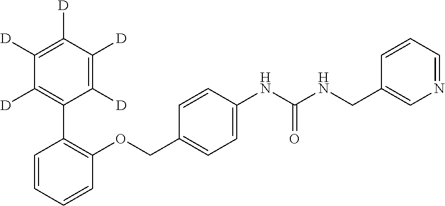 [SMILES: [2H]c1c([2H])c([2H])c(-c2ccccc2OCc2ccc(NC(=O)NCc3cccnc3)cc2)c([2H])c1[2H]] | 8.66 (s, 1H), 8.53 (d, 1H), 8.45 (dd, 1H), 7.72-7.68 (m, 1H), 7.40-7.33 (m, 3H), 7.26- 7.21 (m, 2H), 7.21-7.16 (m, 1H), 7.06-7.00 (m, 1H), 6.71 (t, 1H), 5.02 (s, 2H), 4.32 (d, 2H) |
| 130 | 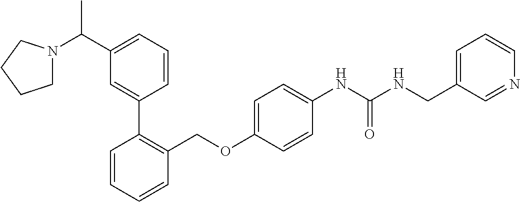 [SMILES: CC(c1cccc(-c2ccccc2COc2ccc(NC(=O)NCc3cccnc3)cc2)c1)N1CCCC1] | 9.93 (s, 1H), 8.71-8.63 (m, 3H), 8.14 (d, 1H), 7.78-7.73 (m, 1H), 7.65-7.60 (m, 1H), 7.56-7.44 (m, 6H), 7.39-7.36 (m, 1H), 7.26 (d, 2H), 6.82 (t, 1H), 6.76 (d, 2H), 4.87 (q, 2H), 4.39 (d, 2H), 4.36-4.29 (m, 1H), 3.69-3.60 (m, 1H), 3.12-3.01 (m, 1H), 2.86 |
| 131 | 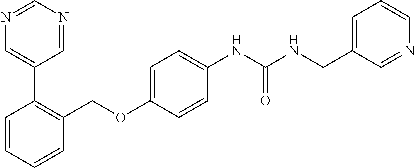 [SMILES: O=C(NCc1cccnc1)Nc1ccc(OCc2ccccc2-c2cncnc2)cc1] | 9.17 (s, 1 H) 8.88 (s, 2 H) 8.51-8.62 (m, 3 H) 7.89 (d, 1 H) 7.64-7.68 (m, 1 H) 7.53 (ddd, 3 H) 7.43-7.46 (m, 1 H) 7.23-7.28 (m, 2 H) 6.73- 6.77 (m, 2 H) 6.69 (t, 1 H) 4.93 (s, 2 H) 4.33 (d, 2 H). |
| 132 | 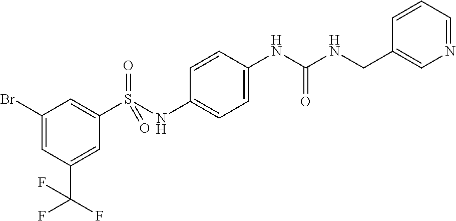 [SMILES: O=C(NCc1cccnc1)Nc1ccc(NS(=O)(=O)c2cc(Br)cc(C(F)(F)F)c2)cc1] | 10.20 (s, 1H), 8.79 (s, 1H), 8.66 (s, 1H), 8.62 (d, 2H), 8.33 (s, 1H), 8.10-8.04 (m, 2H), 7.90 (s, 1H), 7.71-7.65 (m, 1H), 7.30 (d, 2H), 6.92 (d, 2H), 6.92 (t, 1H), 4.37 (d, 2H) |
| 133 | 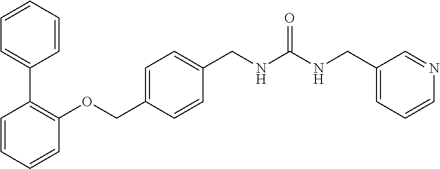 [SMILES: O=C(NCc1ccc(COc2ccccc2-c2ccccc2)cc1)NCc1cccnc1] | 8.52 (s, 1H), 8.49 (dd, 1H), 7.76 (d, 1H), 7.53 (d, 2H), 7.46-7.37 (m, 3H), 7.35-7.28 (m, 4H), 7.24-7.15 (m, 3H), 7.04 (t, 1H), 6.56 (q, 2H), 5.11 (s, 2H), 4.26 (d, 2H), 4.21 (d, 2H) |
| 134 | 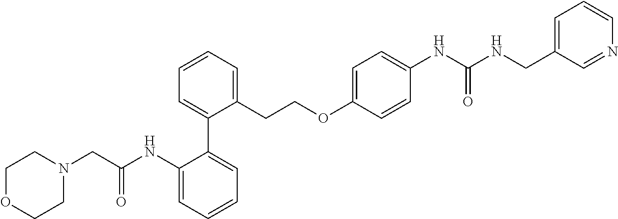 [SMILES: O=C(CN1CCOCC1)Nc1ccccc1-c1ccccc1CCOc1ccc(NC(=O)NCc2cccnc2)cc1] | 8.67 (d, 1H), 8.64 (dd, 1H), 8.57 (s, 1H), 8.08 (dt, 1H), 7.70 (dd, 1H), 7.50-7.37 (m, 3H), 7.35-7.27 (m, 3H), 7.21 (d, 2H), 7.15 (dd, 1H), 6.76 (t, 1H), 6.60 (d, 2H), 4.37 (d, 2H), 3.93-3.86 (m, 2H), 2.89- 2.67 (m, 4H) |
| 135 | 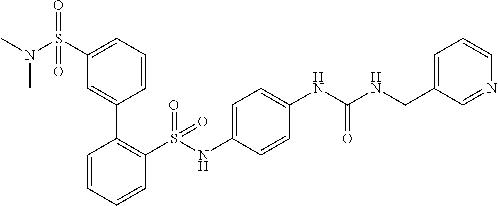 [SMILES: CN(C)S(=O)(=O)c1cccc(-c2ccccc2S(=O)(=O)Nc2ccc(NC(=O)NCc3cccnc3)cc2)c1] | 9.96 (s, 1H), 8.74 (s, 1H), 8.72-8.65 (m, 2H), 8.18 (d, 1H), 8.00-7.96 (m, 1H), 7.80- 7.75 (m, 2H), 7.70-7.59 (m, 4H), 7.52 (d, 1H), 7.39-7.35 (m, 1H), 7.23 (d, 2H), 6.83 (d, 2H), 4.39 (d, 2H), 2.63 (s, 6H) |
| 136 | 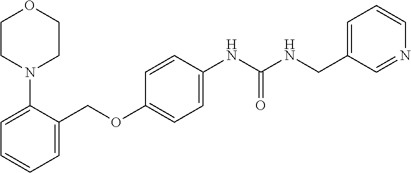 [SMILES: O=C(NCc1cccnc1)Nc1ccc(OCc2ccccc2N2CCOCC2)cc1] | 8.53 (s, 1H), 8.46 (d, 1H), 7.73 (dd, 1H), 7.45 (dd, 1H), 7.38 (dd, 1H), 7.35-7.27 (m, 3H), 7.17 (d, 1H), 7.11 (dd, 1H), 6.88 (d, 2H), 5.07 (s, 2H), 4.31 (s, 2H), 3.71 (dd, 4H), 2.88 (dd, 4H) |
| 137 | 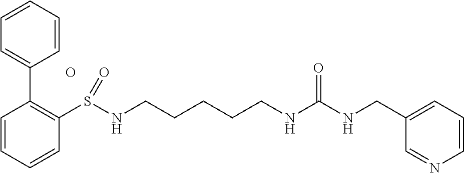 | NA |
| 138 | 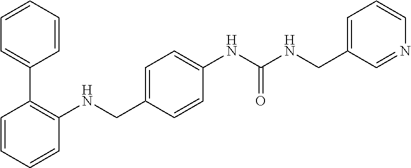 [SMILES: O=C(NCc1cccnc1)Nc1ccc(CNc2ccccc2-c2ccccc2)cc1] | 8.68 (bs, 2H), 8.64 (d, 1H), 8.10 (s, 1H), 7.72 (s, 1H), 7.52-7.40 (m, 5H), 7.37 (d, 1H), 7.32 (d, 2H), 7.19 (d, 2H), 7.06 (t, 1H), 6.97 (d, 1H), 6.76 (t, 1H), 6.64 (t, 1H), 6.54 (t, 1H), 4.39 (d, 2H), 4.20 (s, 2H) |
| 139 | 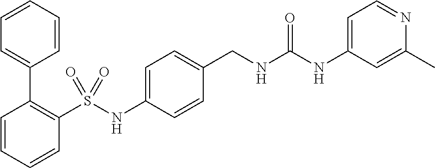 [SMILES: Cc1cc(NC(=O)NCc2ccc(NS(=O)(=O)c3ccccc3-c3ccccc3)cc2)ccn1] | 10.52 (s, 1H), 10.07 (s, 1H), 8.42 (d, 1H), 7.98 (dd, 1.16, 1H), 7.77-7.65 (m, 4H), 7.63 (dd, 2H), 7.56 (dd, 1H), 7.39 (dd, 3H), 7.29 (dd, 1H) 7.25- 7.22 (dd, 1H), 7.25-7.22 (m, 2H), 7.14 (d, 2H), 6.90 (d, 2H), 4.22 (d, 2H), 2.56 (s, 3H) |
| 140 | 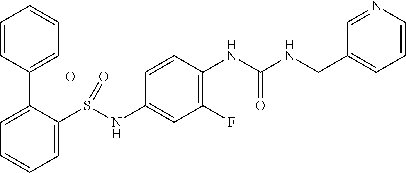 | 10.05 (s, 1H), 8.50 (d, 1H), 8.45 (dd, 1H), 8.28 (d, 1H), 7.98 (dd, 1H), 7.82 (t, 1H), 7.68 (dt, 1H), 7.66-7.55 (m, 2H), 7.39-7.34 (m, 4H), 7.29 (dd, 1H), 7.24-7.21 (m, 2H), 7.01 (t, 1H), 6.70 (dd, 1H), 6.64 (dd, 1H), 4.29 (d, 2H) |
| 141 | 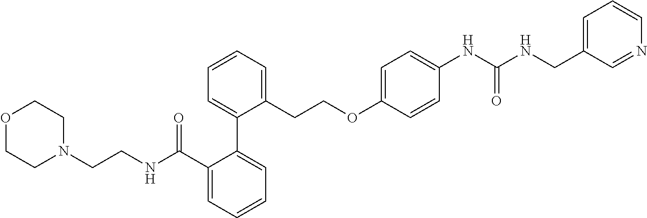 [SMILES: O=C(NCc1cccnc1)Nc1ccc(OCCc2ccccc2-c2ccccc2C(=O)NCCN2CCOCC2)cc1] | 8.51 (d, 1H), 8.45 (dd, 1H), 8.42 (br s, 1H), 7.12-7.67 (m, 1H), 7.53-7.45 (m, 3H), 7.42- 7.39 (m, 1H), 7.35 (ddd, 1H), 7.32-7.26 (m, 2H), 7.23 (dd, 1H), 7.19 (d, 2H), 7.12 (dd, 1H), 6.61-6.58 (m, 3H), 4.29 (d, 2H), 4.02-3.88 (m, 2H), 3.53-3.50 (m, 4H), |
| 142 | 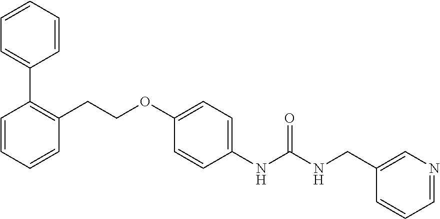 [SMILES: O=C(NCc1cccnc1)Nc1ccc(OCCc2ccccc2-c2ccccc2)cc1] | 8.51 (s, 1), 8.44 (d, 1H), 8.39 (s, 1H), 7.69 (d, 1H), 7.49- 7.15 (m, 12H), 6.63 (d, 2H), 6.59 (t, 2H), 4.29 (d, 2H), 3.96 (t, 2H), 2.96 (t, 2H) |
| 143 | 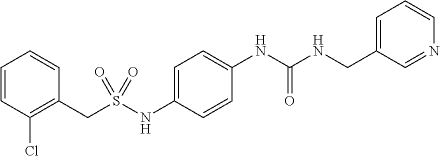 [SMILES: O=C(NCc1cccnc1)Nc1ccc(NS(=O)(=O)Cc2ccccc2Cl)cc1] | 8.79 (s, 1H), 8.71 (d, 1H), 8.52 (d, 1H), 7.99 (dd, 1H), 7.45 (dd, 1H), 7.39 (dd, 1H), 7.35-7.27 (m, 4H), 7.12 (d, 2H), 4.93 (s, 2H), 4.57 (s, 2H) |
| 144 | 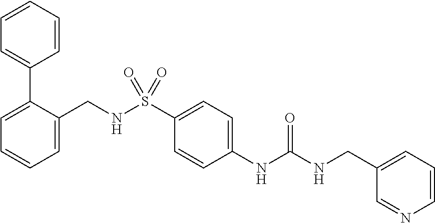 [SMILES: O=C(NCc1cccnc1)Nc1ccc(S(=O)(=O)NCc2ccccc2-c2ccccc2)cc1] | 9.12 (br s, 1H), 8.54 (d, 1H), 8.46 (dd, 1H), 7.85 (t, 1H), 7.72 (dt, 1H), 7.56-7.45 (m, 5H), 7.39-7.29 (m, 6H), 7.27- 7.24 (m, 2H), 7.18 (dd, 1H), 6.92 (t, 1H), 4.34 (d, 2H), 3.82 (d, 2H) |
| 145 | 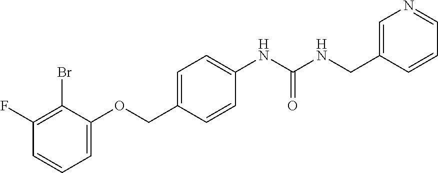 [SMILES: O=C(NCc1cccnc1)Nc1ccc(COc2cccc(F)c2Br)cc1] | 8.71 (s, 1H), 8.52 (d, 1H), 8.45 (dd, 1H), 7.70 (dt, 1H), 7.43 (d, 2H), 7.40-7.32 (m, 4H), 7.05 (d, 1H), 6.96 (td, 1H), 6.73 (t, 1H), 5.13 (s, 2H), 4.32 (d, 2H) |
| 146 | 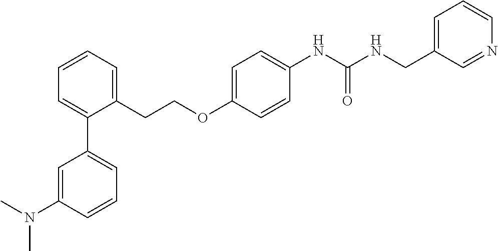 [SMILES: CN(C)c1cccc(-c2ccccc2CCOc2ccc(NC(=O)NCc3cccnc3)cc2)c1] | 8.52 (d, 1H), 8.45 (dd, 1H), 8.39 (s, 1H), 7.70 (dt, 1H), 7.43 (dd, 1H), 7.37 (dd, 1H), 7.32 (d, 1H), 7.29 (t, 1H), 7.28-7.25 (m, 1H), 7.23-7.19 (m, 3H)7.18 (d, 1H), 6.74 (dd, 1H), 6.73 (dd, 1H), 6.65- 6.61 (m, 3H), 6.59-6.57 (m, 1H), 4.29 (d, 2H), 3.97 |
| 147 | 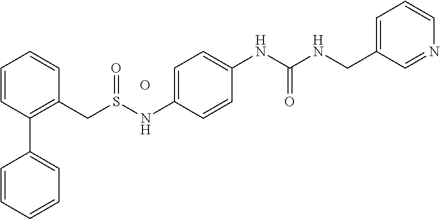 | 9.68 (s, 1H), 8.76 (s, 1H), 8.73-8.72 (m, 2H), 8.70-8.67 (m, 1H), 8.23-8.16 (m, 1H), 7.81-7.75 (m, 1H), 7.53-7.47 (m, 1H), 7.46-7.40 (m, 2H), 7.39-7.22 (m, 8H), 6.90-6.79 (m, 3H), 4.42 (d, 2H), 4.32 (s, 2H) |
| 148 | 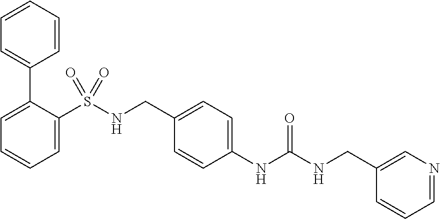 [SMILES: O=C(NCc1cccnc1)Nc1ccc(CNS(=O)(=O)c2ccccc2-c2ccccc2)cc1] | 8.67 (s, 1H), 8.60 (d, 1H), 8.55 (dd, 1H), 7.97 (dd, 1H), 7.91 (dt, 1H), 7.75-7.50 (m, 3H), 7.43-7.26 (m, 7H), 7.03 (d, 2H), 6.74 (t, 1H), 4.35 (d, 2H), 3.79 (d, 2H) |
| 149 | 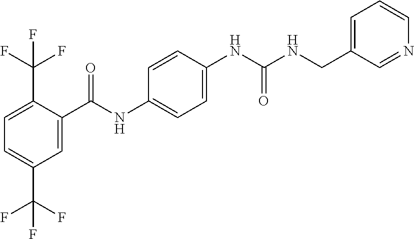 [SMILES: O=C(NCc1cccnc1)Nc1ccc(NC(=O)c2cc(C(F)(F)F)ccc2C(F)(F)F)cc1] | 10.57 (s, 1H), 8.95-8.82 (s, 1H), 8.75 (s, 1H), 8.71 (s, 1H), 8.24 (m, 1H), 8.17-8.07 (m, 3H), 7.82 (m 1H), 7.54 (d, 2H), 7.40 (d, 2H), 6.94 (bs, 1H), 4.43 (d, 2H) |
| 150 | 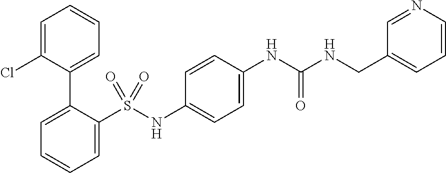 [SMILES: O=C(NCc1cccnc1)Nc1ccc(NS(=O)(=O)c2ccccc2-c2ccccc2Cl)cc1] | 9.88 (s, 1H), 8.65 (s, 1H), 8.58 (d, 2H), 7.96-7.93 (m, 2H), 7.66-7.56 (m, 3H), 7.48 (dd, 1H), 7.39 (td, 1H), 7.30 (td, 1H), 7.24-7.21 (m, 3H), 6.96 (dd, 1H), 6.84 (d, 2H), 6.75 (t, 1H), 4.34 (d, 2H) |
| 151 | 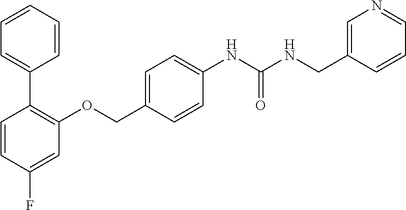 [SMILES: O=C(NCc1cccnc1)Nc1ccc(COc2cc(F)ccc2-c2ccccc2)cc1] | 8.69 (s, 1H), 8.52 (d, 1H), 8.45 (dd, 1H), 7.70 (dt, 1H), 7.47 (d, 2H), 7.40-7.34 (m, 5H), 7.32-7.29 (m, 2H), 7.24 (d, 2H), 7.11 (dd, 1H), 6.86 (td, 1H), 6.74 (t, 1H), 5.04 (s, 2H), 4.31 (d, 2H) |
| 152 | 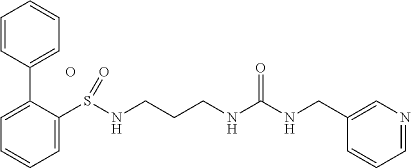 | 8.46-8.41 (m, 2H), 7.92 (d, 1H), 7.68-7.55 (m, 3H), 7.40- 7.29 (m, 7H), 7.25 (t, 1H), 6.43 (t, 1H), 5.97 (t, 1H), 4.20 (d, 2H), 2.95 (q, 2H), 2.68 (dt, 2H, 1.44 (q, 2H) |
| 153 | 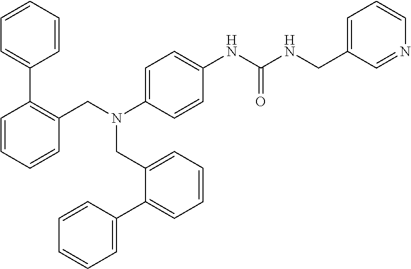 [SMILES: O=C(NCc1cccnc1)Nc1ccc(N(Cc2ccccc2-c2ccccc2)Cc2ccccc2-c2ccccc2)cc1] | 8.58 (s, 1H), 8.55 (d, 1H), 8.17 (s, 1H), 7.91 (bs, 1H), 7.55 (bs, 1H), 7.50-7.31 (m, 14H), 7.30-7.16 (m, 8H), 7.09 (d, 2H), 7.00 (d, 2H), 6.54 (t, 1H), 6.28 (d, 2H), 4.45 9s, 4H), 4.30 (d, 2H) |
| 154 | 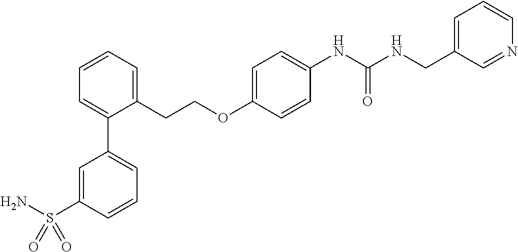 [SMILES: NS(=O)(=O)c1cccc(-c2ccccc2CCOc2ccc(NC(=O)NCc3cccnc3)cc2)c1] | 8.51 (d, 1H), 8.44 (dd, 1H), 8.38 (s, 1H), 7.85 (dt, 1H), 7.80 (br s, 1H), 7.71-7.61 (m, 3H), 7.51 (d, 1H), 7.43-7.33 (m, 5H), 7.22 (d, 3H), 6.69 (d, 2H), 6.58 (t, 1H), 4.29 (d, 2H), 3.99 (t, 2H), 2.96 (t, 2H) |
| 155 | 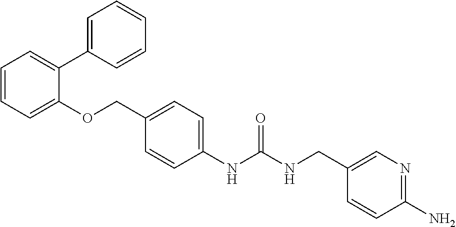 [SMILES: Nc1ccc(CNC(=O)Nc2ccc(COc3ccccc3-c3ccccc3)cc2)cn1] | 8.56 (s, 1H), 7.84 (s, 1H), 7.51 (d, 2H), 7.37-7.33 (m, 5H), 7.33-7.27 (m, 3H), 7.26- 7.16 (m, 3H), 6.50 (m, 2H), 6.18 (s, 2H), 5.01 (s, 2H), 4.09 (d, 2H) |
| 156 | 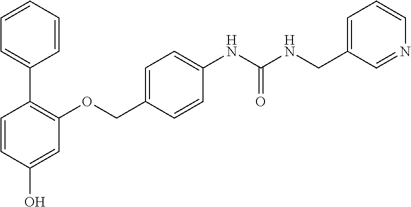 [SMILES: O=C(NCc1cccnc1)Nc1ccc(COc2cc(O)ccc2-c2ccccc2)cc1] | 9.56 (s, 1H), 8.75 (s, 1H), 8.64 (d 1H), 8.59 (dd, 1H), 8.00 (d, 1H), 7.61 (s, 1H), 7.45 (d, 2H), 7.41-7.18 (m, 7H), 7.10 (d, 1H), 6.78 t, 1H), 6.57 (d, 1H), 6.45 (dd, 1H), 4.96 (s, 2H), 4.37 (d, 2H) |
| 157 | 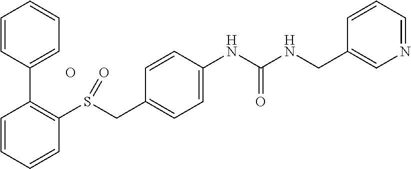 | 8.77 (s, 1H), 8.62 (d, 1H), 8.57 (dd, 1H), 7.96 (bs, 1H), 7.84 (d, 1H), 7.75 (td, 1H), 7.59 (td), 2H), 7.51-7.40 (m, 6H), 7.28 (d, 2H), 6.81 (d, 2H), 6.79 (t, 1H), 4.35 (d, 2H), 4.06 (s, 2H) |
| 158 | 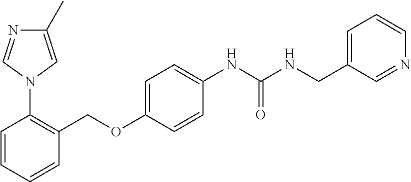 [SMILES: Cc1cn(-c2ccccc2COc2ccc(NC(=O)NCc3cccnc3)cc2)cn1] | 9.31 (s, 1H), 8.60 (s, 1H), 8.03 (d, 1H), 7.76 (d, 1H), 7.75 (s, 1H), 7.72-7.58 (m, 4H), 7.28 (d, 2H), 6.80 (d, 2H), 6.75 (t, 1H), 5.00 (s, 2H), 4.37 (d, 2H), 2.30 (s, 3H) |
| 159 | 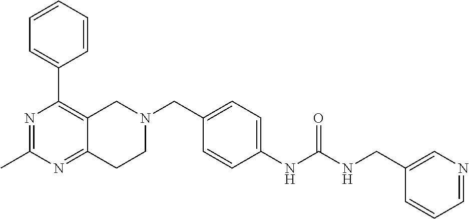 [SMILES: Cc1nc2c(c(-c3ccccc3)n1)CN(Cc1ccc(NC(=O)NCc3cccnc3)cc1)CC2] | 9.09 (s, 1H), 8.69 (s, 1H), 8.65 (d, 1H), 8.09 (d, 1H), 7.74-7.68 (m, 1H), 7.61-7.54 (m, 5H), 7.45 (d, 2H), 7.32 (d, 2H), 7.08 (t, 1H), 4.66- 4.20 (m, 6H), 3.75-3.36 (m, 2H), 3.29-3.07 (m, 2H), 2.65 (s, 3H) |
| 160 | 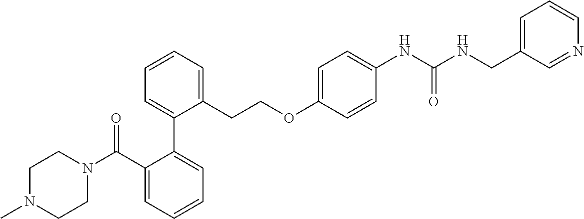 [SMILES: CN1CCN(C(=O)c2ccccc2-c2ccccc2CCOc2ccc(NC(=O)NCc3cccnc3)cc2)CC1] | 8.64 (d, 1H), 8.60 (dd, 1H), 8.56 (s, 1H), 8.01 (dt, 1H), 7.63 (dd, 1H), 7.55-7.48 (m, 3H), 7.35 (t, 1H), 7.30-7.13 (m, 3H), 6.78 (t, 1H), 6.60 (br s, 1H), 4.36 (d, 2H), 3.58- 2.93 (m, 4H), 2.89-2.72 (m, 4H) |
| 161 | 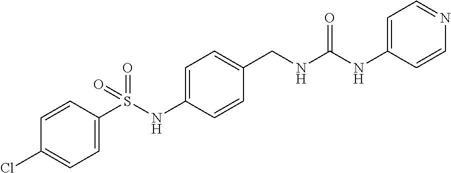 [SMILES: O=C(NCc1ccc(NS(=O)(=O)c2ccc(Cl)cc2)cc1)Nc1ccncc1] | 10.46 (s, 1H), 10.37 (s, 1H), 8.53 (s, 2H), 7.84 (s, 2H), 7.74 (d, 2H), 7.66-7.54 (m, 4H), 7.38 (d, 1H), 7.18 (d, 2H), 7.05 (d, 2H), 4.24 (d, 2H) |
| 162 | 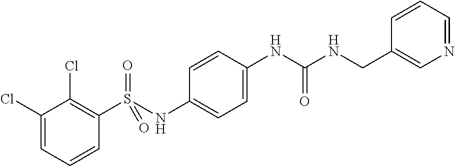 [SMILES: O=C(NCc1cccnc1)Nc1ccc(NS(=O)(=O)c2cccc(Cl)c2Cl)cc1] | 10.43 (s, 1H), 8.56 (s, 1H), 8.49 (s, 1H), 8.46-8.39 (m, 1H), 7.99-7.82 (m, 2H), 7.66 (d, 1H), 7.49 (t, 1H), 7.38- 7.29 (m, 1H), 7.25 (d, 2H), 6.96 (d, 2H), 6.70-6.59 (m, 1H), 4.27 (d, 2H) |
| 163 | 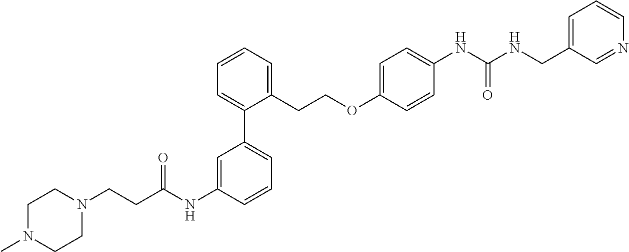 [SMILES: CN1CCN(CCC(=O)Nc2cccc(-c3ccccc3CCOc3ccc(NC(=O)NCc4cccnc4)cc3)c2)CC1] | 10.17 (s, 1H), 8.51 (d, 1H), 8.45 (dd, 1H), 8.43 (s, 1H), 7.69 (dt, 1H), 7.64 (br s, 1H), 7.56 (d, 1H), 7.45 (d, 1H), 7.40-7.28 (m, 4H), 7.23-7.17 (m, 3H), 7.02 (d, 1H), 6.66- 6.60 (m, 3H), 4.29 (d, 2H), 3.95 (t, 2H), 2.96 (t, 2H), 2.78 (m, 4H), 2.34 (m, |
| 164 | 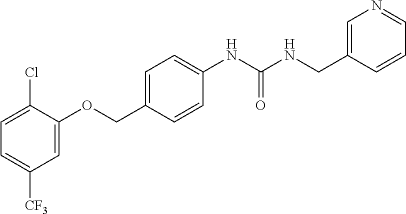 [SMILES: O=C(NCc1cccnc1)Nc1ccc(COc2cc(C(F)(F)F)ccc2Cl)cc1] | 8.73 (s, 1H), 8.52 (d, 1H), 8.45 (dd, 1H), 7.70-7.56 (m, 2H), 7.56 (d, 1H), 7.44 (d, 2H), 7.38-7.35 (m, 4H), 6.74 (t, 1H), 5.21 (s, 2H), 4.32 (d, 2H) |
| 165 | 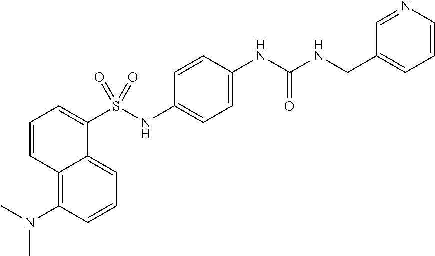 [SMILES: CN(C)c1cccc2c(S(=O)(=O)Nc3ccc(NC(=O)NCc4cccnc4)cc3)cccc12] | 10.32 (br s, 1H), 8.66 (s, 1H), 8.64 (s, 2H), 8.40 (dd, 2H), 8.13-8.09 (m, 2H), 7.75-7.72 (m, 1H), 7.63-7.53 (m, 2H), 7.25 (d, 1H), 7.15 (d, 2H), 6.75 (t, 1H), 4.34 (d, 2H) |
| 166 | 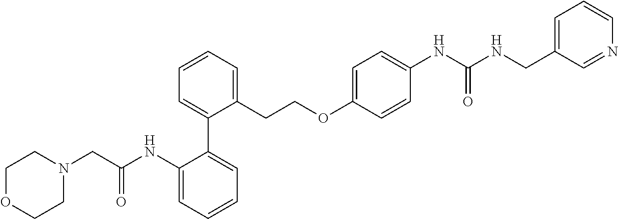 [SMILES: O=C(CN1CCOCC1)Nc1ccccc1-c1ccccc1CCOc1ccc(NC(=O)NCc2cccnc2)cc1] | 8.67 (d, 1H), 8.64 (dd, 1H), 8.57 (s, 1H), 8.08 (dt, 1H), 7.70 (dd, 1H), 7.50-7.37 (m, 3H), 7.35-7.27 (m, 3H), 7.21 (d, 2H), 7.15 (dd, 1H), 6.76 (t, 1H), 6.60 (d, 2H), 4.37 (d, 2H), 3.93-3.86 (m, 2H), 2.89- 2.67 (m, 4H) |
| 167 | 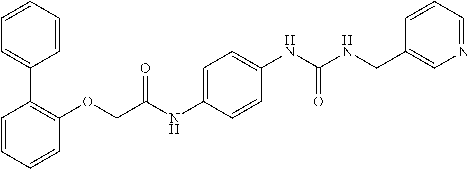 [SMILES: O=C(COc1ccccc1-c1ccccc1)Nc1ccc(NC(=O)NCc2cccnc2)cc1] | 9.69 (s, 1H), 8.61 (s, 1H), 8.50 (d, 1H), 8.44 (d, 1H), 7.70 (s, 1H), 7.67-7.57 (m, 3H), 7.48-7.25 (m, 7H), 7.10- 7.01 (m, 3H), 6.71 (bs, 1H), 4.67 (s, 2H), 4.29 (d, 2H) |
| 168 | 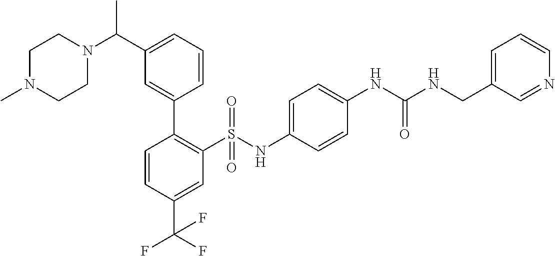 [SMILES: CC(c1cccc(-c2ccc(C(F)(F)F)cc2S(=O)(=O)Nc2ccc(NC(=O)NCc3cccnc3)cc2)c1)N1CCN(C)CC1] | 10.08 (s, 1H), 8.75 (s, 1H), 8.63-8.56 (m, 2H), 8.32-8.27 (m, 1H), 8.04-7.99 (m, 1H), 7.96 (d, 1H), 7.63-7.54 (m, 2H), 7.44-7.36 (m, 2H), 7.30- 7.22 (m, 4H), 7.20-7.15 (m, 1H), 6.87-6.81 (m, 3H), 4.34 (d, 2H), 3.74-3.60 (m, 1H), 3.49-2.66 (m, 11H), 1.36 (d |
| 169 | 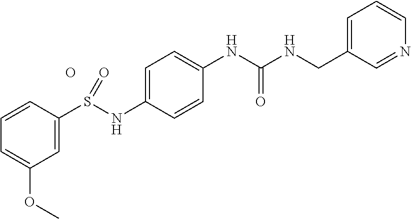 | 9.92 (s, 1H), 8.57 (s, 1H), 8.49 (d, 1H), 8.45-8.43 (m, 1H), 7.70-7.65 (m, 1H), 7.43 (t, 1H), 7.36-7.32 (m, 1H), 7.27-7.13 (m, 5H), 6.93 (d, 2H), 6.66 (t, 1H), 4.28 (d, 2H), 3.75 (s, 3H) |
| 170 | 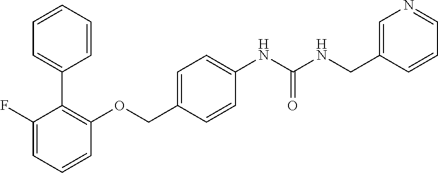 [SMILES: O=C(NCc1cccnc1)Nc1ccc(COc2cccc(F)c2-c2ccccc2)cc1] | 8.65 (s, 1H), 8.51 (d, 1H), 8.45 (dd, 1H), 7.69 (dt, 1H), 7.44-7.32 (m, 8H)7.17 (d, 2H), 7.04 (d, 1H), 6.90 (td, 1H), 6.71 (t, 1H), 5.01 (s, 2H), 4.30 (d, 2H) |
| 171 | 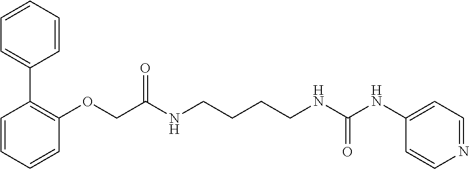 [SMILES: O=C(COc1ccccc1-c1ccccc1)NCCCCNC(=O)Nc1ccncc1] | 10.20 (bs, 1H), 8.51 (d, 2H), 7.81 (d, 2H), 7.71 (t, 1H), 7.59 (d, 3H), 7.42 (t, 3H), 7.36-7.29 (m, 3H), 7.10-6.96 (m, 3H), 6.57 (bs, 3H), 4.48 (s, 2H), 3.13 (bs, 4H), 1.42 (bs, 4H) |
| 172 | 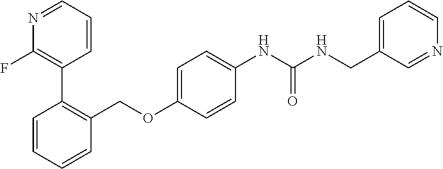 [SMILES: O=C(NCc1cccnc1)Nc1ccc(OCc2ccccc2-c2cccnc2F)cc1] | 8.69 (s, 1 H) 8.67 (d, 1 H) 8.58 (s, 1 H) 8.24 (d, 1 H) 8.16 (d, 1 H) 7.95 (ddd, 1 H) 7.76 (dd, 1 H) 7.60-7.64 (m, 1 H) 7.44-7.52 (m, 2 H) 7.41 (ddd, 1 H) 7.34-7.38 (m, 1 H) 7.19-7.26 (m, 2 H) 6.77 (t, 1 H) 6.63-6.71 (m, 2 H) 4.85 (s, 2 H) 4.38 |
| 173 | 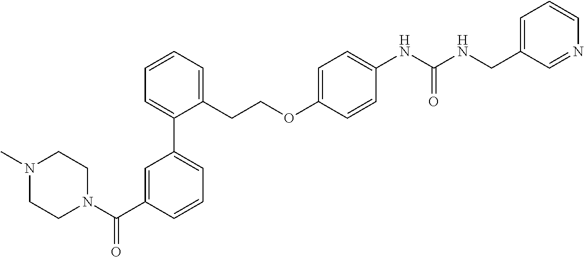 [SMILES: CN1CCN(C(=O)c2cccc(-c3ccccc3CCOc3ccc(NC(=O)NCc4cccnc4)cc3)c2)CC1] | 8.62 (d, 1H), 8.58 (dd, 1H), 8.53 (s, 1H), 7.97 (br d, 1H), 7.62-7.55 (m, 2H), 7.52-7.44 (m, 4H), 7.40-7.31 (m, 2H), 7.25-7.21 (m, 3H), 6.73 (t, 1H), 6.66 (d, 2H), 4.35 (d, 2H), 3.97 (t, 2H), 3.34-3.06 (m, 2H), 2.98 (t, 2H), 2.78 (s, 3H) |
| 174 | 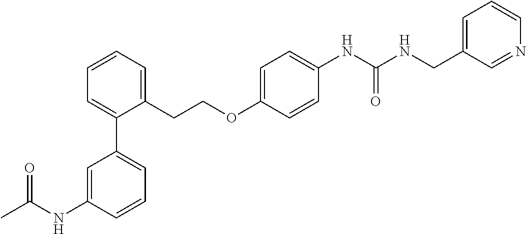 [SMILES: CC(=O)Nc1cccc(-c2ccccc2CCOc2ccc(NC(=O)NCc3cccnc3)cc2)c1] | 10.03 (br s, 1H), 8.52 (d, 1H), 8.46 (dd, 1H), 8.40 (s, 1H), 7.72 (dt, 1H), 7.62 (t, 1H), 7.58-7.55 (m, 1H), 7.45 (dd, 1H), 7.40-7.28 (m, 4H), 7.21 (d, 2H), 7.18 (dd, 1H), 7.01 (dt, 1H), 6.65 (d, 2H), 6.59 (t, 1H), 4.29 (d, 2H), 3.95 (t, 2H), 2.95 (t, 2 |
| 175 | 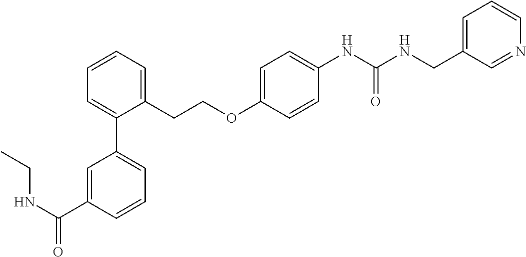 [SMILES: CCNC(=O)c1cccc(-c2ccccc2CCOc2ccc(NC(=O)NCc3cccnc3)cc2)c1] | 8.54 (d, 1H), 8.47 (dd, 1H), 8.38 (s, 1H), 7.88-7.83 (m, 2H), 7.70 (m, 1H), 7.56-7.49 (m, 4H), 7.39-7.33 (m, 3H), 7.26-7.19 (m, 2H), 6.68-6.64 (m, 2H), 6.59 (t, 1H), 4.30 (d, 2H), 3.98 (t, 2H), 3.30 (q, 2H), 2.96 (t, 2H), 1.13 (t, 3H) |
| 176 | 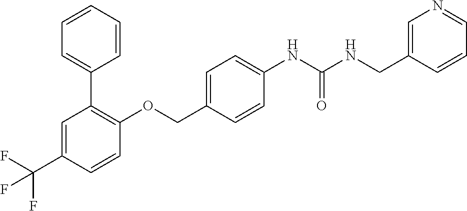 [SMILES: O=C(NCc1cccnc1)Nc1ccc(COc2ccc(C(F)(F)F)cc2-c2ccccc2)cc1] | 8.70 (s, 1H), 8.52 (d, 1H), 8.45 (dd, 1H), 7.70 (dt, 1H), 7.58-7.53 (m, 3H), 7.44-7.33 (m, 7H), 7.25 (d, 2H), 6.75 (t, 1H), 5.13 (s, 2H), 4.31 (d, 2H) |
| 177 | 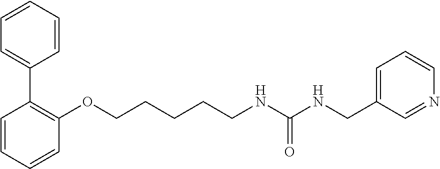 [SMILES: O=C(NCCCCCOc1ccccc1-c1ccccc1)NCc1cccnc1] | n/a |
| 178 | 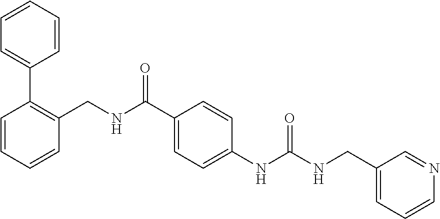 [SMILES: O=C(NCc1cccnc1)Nc1ccc(C(=O)NCc2ccccc2-c2ccccc2)cc1] | 8.99 (s, 1H), 8.774 (t, 1H), 8.53 (d, 1H), 8.46 (dd, 1H), 7.77 (d, 2H), 7.71 (dt, 1H), 7.51-7.29 (m, 10H), 7.22 (dd, 1H), 6.90 (t, 1H), 4.39 (d, 2H), 4.33 (d, 2H) |
| 179 | 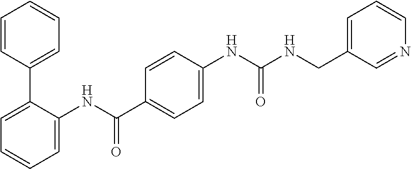 [SMILES: O=C(NCc1cccnc1)Nc1ccc(C(=O)Nc2ccccc2-c2ccccc2)cc1] | 9.61 (s, 1H), 9.09 (s, 1H), 8.69 (d, 1H), 8.64 (dd, 1H), 8.11 (d, 1H), 7.73-7.65 (m, 3H), 7.50-7.29 (m, 11H), 6.96 (t, 1H, 4.41 (d, 2H) |
| 180 | 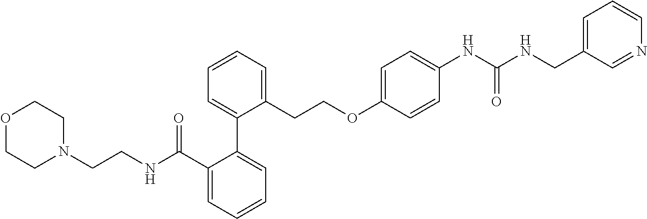 [SMILES: O=C(NCc1cccnc1)Nc1ccc(OCCc2ccccc2-c2ccccc2C(=O)NCCN2CCOCC2)cc1] | 8.51 (d, 1H), 8.45 (dd, 1H), 8.42 (br s, 1H), 7.12-7.67 (m, 1H), 7.53-7.45 (m, 3H), 7.42- 7.39 (m, 1H), 7.35 (ddd, 1H), 7.32-7.26 (m, 2H), 7.23 (dd, 1H), 7.19 (d, 2H), 7.12 (dd, 1H), 6.61-6.58 (m, 3H), 4.29 (d, 2H), 4.02-3.88 (m, 2H), 3.53-3.50 (m, 4H), |
| 181 | 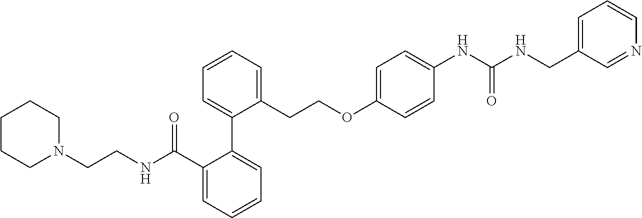 [SMILES: O=C(NCc1cccnc1)Nc1ccc(OCCc2ccccc2-c2ccccc2C(=O)NCCN2CCCCC2)cc1] | 9.13 (br s, 1H), 8.67 (d, 1H), 8.65 (dd, 1H), 8.58 (s, 1H), 8.46 (t, 1H), 8.09 (dt, 1H), 7.71 (dd, 1H), 7.56-7.47 (m, 2H), 7.41 (dd, 1H), 7.33-7.29 (m, 2H), 7.26-7.19 (m, 3H), 7.12 (dd, 1H), 6.78 (t, 1H), 6.60 (d, 2H), 4.37 (d, 2H), 4.02-3.89 (m, 4H), 3 |
| 182 | 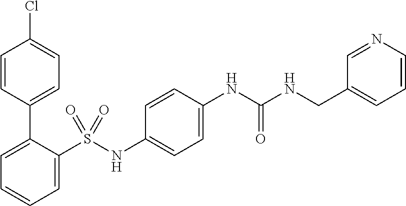 [SMILES: O=C(NCc1cccnc1)Nc1ccc(NS(=O)(=O)c2ccccc2-c2ccc(Cl)cc2)cc1] | 9.79 (br s, 1H), 8.53 (br s, 1H), 8.49 (d, 1H), 8.44 (dd, 1H), 7.96 (dd, 1H), 7.67 (dt, 1H), 7.64-7.54 (m, 2H), 7.43 (d, 2H), 7.34 (ddd, 1H), 7.27 (dd, 1H), 7.21 (d, 2H), 6.79 (d, 2H), 6.65 (t, 1H), 4.28 (d, 2H) |
| 183 | 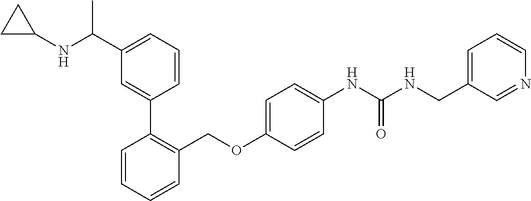 [SMILES: CC(NC1CC1)c1cccc(-c2ccccc2COc2ccc(NC(=O)NCc3cccnc3)cc2)c1] | 8.70-8.61 (m, 3H), 8.11 (d, 1H), 7.75-7.69 (m, 1H), 7.65- 7.59 (m, 1H), 7.58-7.53 (m, 1H), 7.53-7.43 (m, 6H), 7.38- 7.33 (m, 1H), 7.25 (d, 2H), 6.83 (t, 1H), 6.76 (d, 2H), 4.88 (q, 2H), 4.49-4.32 (m, 3H), 2.43-2.31 (m, 1H), 1.51 (d, 3H), 0.81-0.53 (m, 4H) |
| 184 | 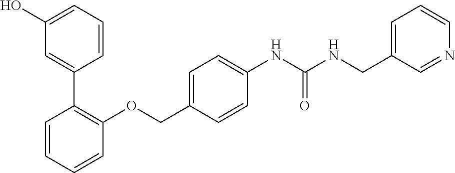 [SMILES: O=C(NCc1cccnc1)Nc1ccc(COc2ccccc2-c2cccc(O)c2)cc1] | 9.38 (s, 1H), 8.66 (s, 1H), 8.52 (d, 1H), 8.45 (dd, 1H), 7.70 (dt, 1H), 7.39-7.34 (m, 3H), 7.31-7.23 (m, 4H), 7.19- 7.14 (m, 2H), 7.00 (td, 1H), 6.93-6.89 (m, 2H), 6.72-6.68 (m, 2H), 5.00 (s, 2H), 4.31 (d, 2H), |
| 185 | 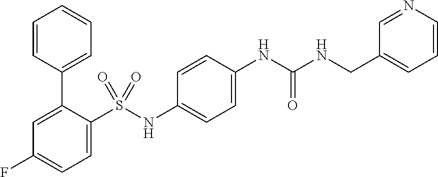 [SMILES: O=C(NCc1cccnc1)Nc1ccc(NS(=O)(=O)c2ccc(F)cc2-c2ccccc2)cc1] | 9.81 (s, 1H), 8.75 (s, 1H), 8.69-8.65 (m, 2H), 8.13 (d, 1H), 8.02-7.98 (m, 1H), 7.79-7.72 (m, 1H), 7.45-7.34 (m, 4H), 7.25-7.15 (m, 5H), 6.85-6.77 (m, 3H), 4.38 (d, 2H) |
| 186 | 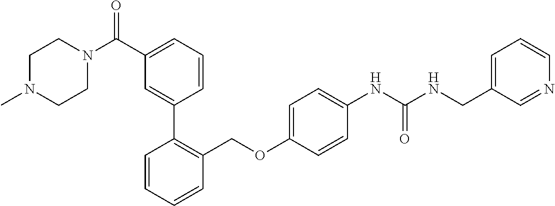 [SMILES: CN1CCN(C(=O)c2cccc(-c3ccccc3COc3ccc(NC(=O)NCc4cccnc4)cc3)c2)CC1] | 8.74-8.58 (m, 3 H), 8.07- 7.98 (m, 1 H), 7.68-7.57 (m, 2 H), 7.57-7.50 (m, 2 39 H), 7.50-7.42 (m, 4 H), 7.39-7.33 (m, 1 H), 7.29-7.20 (m, 2 H), 6.94-6.85 (m, 1 H), 6.81- 6.69 (m, 2 H), 4.88 (s, 2 H), 4.36 (d, 2 H), 3.75-2.99 (m, 8 H), 2.77 (s, 3 |
| 187 | 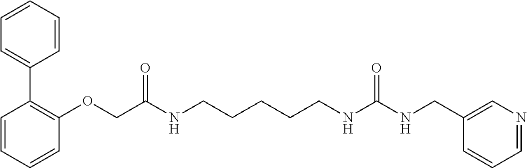 [SMILES: O=C(COc1ccccc1-c1ccccc1)NCCCCCNC(=O)NCc1cccnc1] | 8.57-8.52 (m, 3H), 7.85 (bs, 1H), 7.66-7.57 (m, 4H), 7.53 (bs, 1H), 7.47-7.38 (m, 3H), 7.38-7.30 (m, 3H), 7.07 (5, 1H), 7.00 (d, 1H), 6.42 (t, 1H), 6.06 (t, 1H), 4.48 (s, 2H), 4.25 (d, 2H), 3.11-3.04 (m, 2H), 3.01-2.93 (m, 2H), 1.42-1.28 (m, 3H), 1.27-1.1 |
| 188 | 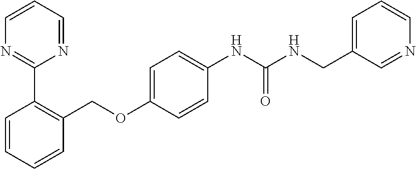 [SMILES: O=C(NCc1cccnc1)Nc1ccc(OCc2ccccc2-c2ncccn2)cc1] | 8.91 (d, 2 H) 8.76 (s, 1 H) 8.74 (d, 1 H) 8.65 (s, 1 H) 8.32 (d, 1 H) 7.95-8.00 (m, 1 H) 7.89 (dd, 1 H) 7.65 (d, 1 H) 7.44-7.54 (m, 3 H) 7.20- 7.30 (m, 2 H) 6.84 (t, 1 H) 6.67-6.77 (m, 2 H) 5.45 (s, 2 H) 4.42 (d, 2 H). |
| 189 | 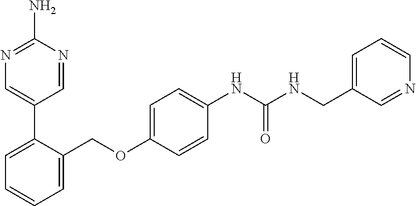 [SMILES: Nc1ncc(-c2ccccc2COc2ccc(NC(=O)NCc3cccnc3)cc2)cn1] | 8.80-8.76 (m, 2H), 8.73 (s, 1H), 8.40 (s, 2H), 8.39-8.36 (bd, 1H), 7.94 (dd, 1H), 7.60 (dd, 1H), 7.49-7.41 (m, 2H), 7.37 (dd, 1H), 7.29 (d, 2H), 6.90 (t, 3H), 6.84 (d, 2H), 4.99 (s, 2H), 4.40 (d, 2H). |
| 190 | 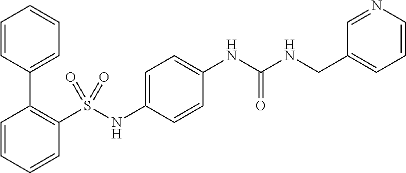 [SMILES: O=C(NCc1cccnc1)Nc1ccc(NS(=O)(=O)c2ccccc2-c2ccccc2)cc1] | 9.78 (br s, 1H), 8.53 (s, 1H), 8.50-8.49 (m, 1H), 8.44 (dd, 1H), 7.94 (dd, 1H), 7.69-7.66 (m, 1H), 7.61 (td, 1H), 7.54 (td, 1H), 7.39-7.33 (m, 4H), 7.28 (dd, 1H), 7.24-7.21 (m, 4H), 6.82 (d, 2H), 6.65 (t, 1H), 4.28 (d, 2H) |
| 191 | 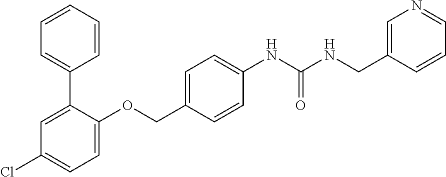 [SMILES: O=C(NCc1cccnc1)Nc1ccc(COc2ccc(Cl)cc2-c2ccccc2)cc1] | 8.67 (s, 1H), 8.52 (d, 1H), 8.45 (dd, 1H), 7.70 (dt, 1H), 7.52 (d, 2H), 7.42-7.31 (m, 7H), 7.21 (dd, 3H), 6.73 (t, 1H), 5.02 (s, 2H), 4.31 (d, 2H) |
| 192 | 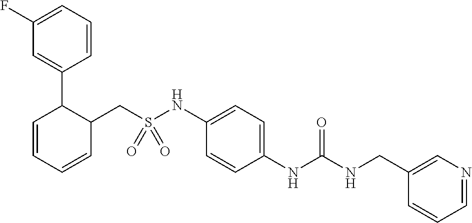 [SMILES: O=C(NCc1cccnc1)Nc1ccc(NS(=O)(=O)CC2C=CC=CC2c2cccc(F)c2)cc1] | 9.67 (s, 1H), 8.71 (s, 1H), 8.67 (s, 1H), 8.62 (d, 1H), 8.07 (d, 1H), 7.67 (dd, 1H), 7.53-7.48 (m, 1H), 7.47-7.42 (m, 2H), 7.41-7.35 (m, 1H), 7.31-7.23 (m, 3H), 7.18-7.09 (m, 3H), 6.87 (d, 2H), 6.77 (t, 1H), 4.39 (d, 2H), 4.31 (s, 2H) |
| 193 | 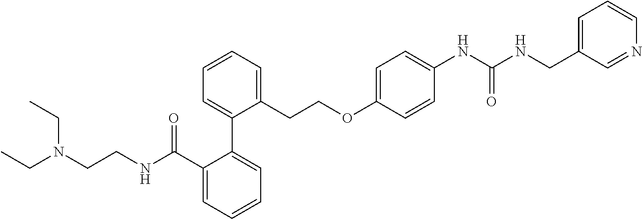 [SMILES: CCN(CC)CCNC(=O)c1ccccc1-c1ccccc1CCOc1ccc(NC(=O)NCc2cccnc2)cc1] | 9.17 (br s, 1H), 8.65 (d, 1H), 8.62 (dd, 1H), 8.57 (s, 1H), 8.42 (t, 1H), 8.04 (dt, 1H), 7.68-7.65 (m, 1H), 7.57-7.47 (m, 3H), 7.41 (dd, 1H), 7.33- 7.28 (m, 2H), 7.25-7.19 (m, 3H), 7.11 (dd, 1H), 6.77 (t, 1H), 6.61 (d, 2H), 4.36 (d, 2H), 4.01-4.89 (m, 2H |
| 194 | 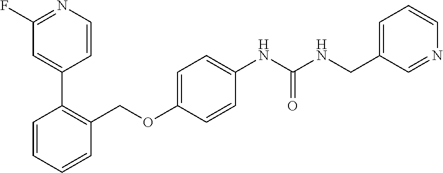 [SMILES: O=C(NCc1cccnc1)Nc1ccc(OCc2ccccc2-c2ccnc(F)c2)cc1] | 8.51 (s, 1 H) 8.40-8.47 (m, 2 H) 8.26 (d, 1 H) 7.67-7.73 (m, 1 H) 7.61-7.67 (m, 1 H) 7.46-7.55 (m, 2 H) 7.40- 7.45 (m, 2 H) 7.31-7.39 (m, 1 H) 7.20-7.29 (m, 3 H) 6.72- 6.80 (m, 2 H) 6.56-6.66 (m, 1 H) 4.94 (s, 2 H) 4.29 (d, 2 H). |
| 195 | 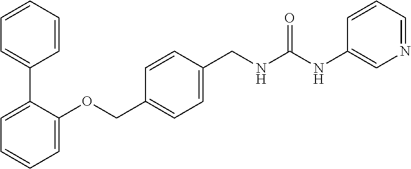 [SMILES: O=C(NCc1ccc(COc2ccccc2-c2ccccc2)cc1)Nc1cccnc1] | 9.01 (bs, 1H), 8.70 (bs, 1H), 8.21 (d, 1H), 8.01 (dt, 1H), 7.53 (d, 2H), 7.40 (t, 2H), 7.36-7.26 (m, 8H), 7.18 (d, 1H), 7.04 (t, 1H), 6.92 (bs, 1H), 5.11 (s, 2H), 4.30 (d, 1H) |
| 196 | 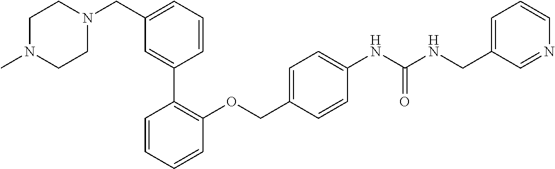 [SMILES: CN1CCN(Cc2cccc(-c3ccccc3OCc3ccc(NC(=O)NCc4cccnc4)cc3)c2)CC1] | 8.68 (s, 1H), 8.53-8.50 (m, 1H), 8.46-8.43 (m, 1H), 7.72- 7.67 (m, 1H), 7.45-7.42 (m, 1H), 7.39-7.16 (m, 11H), 7.06- 7.00 (m, 1H), 6.71 (t, 1H), 5.01 (s, 2H), 4.31 (d, 2H), 3.43 (s, 2H), 2.47-2.15 (m, 8H), 2.11 (s, 3H) |
| 197 | 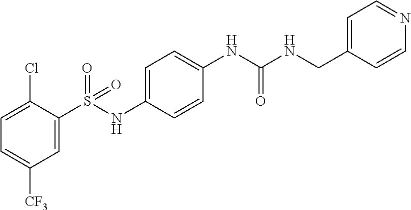 [SMILES: O=C(NCc1ccncc1)Nc1ccc(NS(=O)(=O)c2cc(C(F)(F)F)ccc2Cl)cc1] | 10.88 (s, 1H), 10.58 (s, 1H), 8.52 (d, 2H), 8.21 (s, 1H), 8.06-8.01 (m, 1H), 7.92 (d, 1H), 7.85-7.78 (m, 3H), 7.71 (t, 1H), 7.19 (d, 2H), 7.08 (d, 2H), 4.23 (d, 2H) |
| 198 | 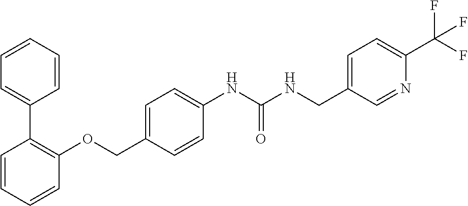 [SMILES: O=C(NCc1ccc(C(F)(F)F)nc1)Nc1ccc(COc2ccccc2-c2ccccc2)cc1] | 8.78 (s, 1H), 8.70 (s, 1H), 7.98 (d, 1H), 7.88 (d, 1H), 7.51 (d, 2H), 7.42-7.35 (m, 4H), 7.35-7.27 (m, 3H), 7.24 (d, 2H), 7.19 (d, 1H), 7.03 (dd, 1H), 6.83 (dd, 1H), 5.02 (s, 2H), 4.42 (d, 2H) |
| 199 | 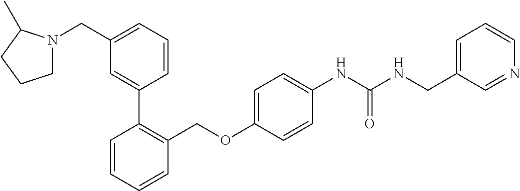 [SMILES: CC1CCCN1Cc1cccc(-c2ccccc2COc2ccc(NC(=O)NCc3cccnc3)cc2)c1] | 9.49 (br s, 1 H), 8.76-8.45 (m, 2 H), 7.96 (d, 1 H), 7.67- 7.43 (m, 8 H), 7.39-7.33 (m, 1 H), 7.30-7.19 (m, 2 H), 6.84-6.68 (m, 3 H), 4.94- 4.82 (m, 2 H), 4.55-4.44 (d, 1 H), 4.34 (d, 2 H), 4.17- 4.05 (m, 1 H), 3.47-3.31 (m, 1 H), 3.20-3.1 |
| 200 | 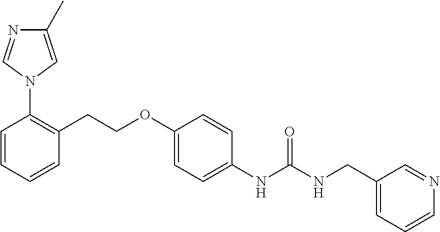 [SMILES: Cc1cn(-c2ccccc2CCOc2ccc(NC(=O)NCc3cccnc3)cc2)cn1] | 9.32 (s, 1H), 8.55 (s, 1H), 8.00 (d, 1H), 7.78-7.45 (m, 4H), 7.27 (d, 2H), 6.74 (d, 2H), 4.36 (, d, 2H), 4.07 (t, 2H), 2.89 (t, 2H), 2.35 (s, 3H) |
| 201 | 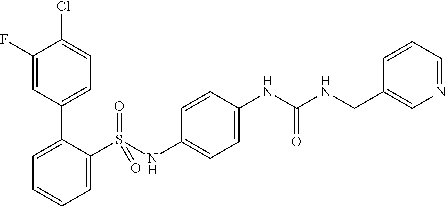 [SMILES: O=C(NCc1cccnc1)Nc1ccc(NS(=O)(=O)c2ccccc2-c2ccc(Cl)c(F)c2)cc1] | 9.80 (s, 1H), 8.55 (s, 1H), 8.50-8.47 (m, 1H), 8.46-8.41 (s, 1H), 8.02-7.95 (m, 1H), 7.71-7.53 (m, 4H), 7.39-7.28 (m, 2H), 7.22 (d, 2H) 7.16 (d, 1H), 7.01 (d, 1H), 6.78 (d, 2H), 6.65 (t, 1H), 4.28 (d, 2H) |
| 202 | 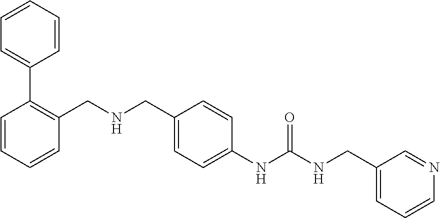 [SMILES: O=C(NCc1cccnc1)Nc1ccc(CNCc2ccccc2-c2ccccc2)cc1] | 9.17 (s, 1H), 8.96 (s, 1H), 8.68 (s, 1H), 8.62 (d, 1H), 8.05 (d, 1H), 7.74-7.62 (m, 2H), 7.54-7.46 (m, 2H), 7.42- 7.28 (m, 5H), 7.26 (dd, 2H), 7.20 (d, 2H), 6.98 (t, 1H), 4.40 (d, 2H), 4.06 (t, 2H), 4.00 (t, 2H) |
| 203 | 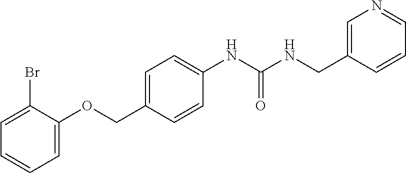 [SMILES: O=C(NCc1cccnc1)Nc1ccc(COc2ccccc2Br)cc1] | 8.71 (s, 1H), 8.52 (d, 1H), 8.45 (dd, 1H), 7.71 (dt, 1H), 7.57 (dd, 1H), 7.42 (dd, 2H), 7.37-7.18 (m, 4H), 7.18 (dd, 1H), 5.09 (s, 2H), 4.32 (d, 2H) |
| 204 | 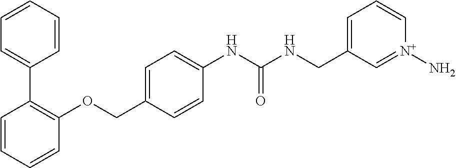 [SMILES: N[n+]1cccc(CNC(=O)Nc2ccc(COc3ccccc3-c3ccccc3)cc2)c1] | 9.30 (s, 1H), 8.71 (s, 1H), 8.69 (s, 1H), 8.63 (s, 1H), 8.17 (d, 1H), 7.97 (dd, 1H), 7.51 (d, 2H), 7.42-7.36 (m, 4H), 7.35-7.27 (m, 3H), 7.24 (d, 2H), 7.18 (d, 1H), 7.03 (dd, 1 h), 5.02 (s, 2H), 4.45 (m, 2 h), 3.32 (s, 1H) |
| 205 | 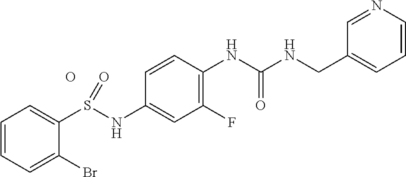 | 10.61 (s, 1H), 8.49 (d, 1H), 8.44 (dd, 1H), 8.3-8.28 (m, 1H), 8.04 (dd, 1H), 7.87-7.81 (m, 2H), 7.66 (dt, 1H), 7.58- 7.50 (m, 2H), 7.34 (ddd, 1H), 7.02 (t, 1H), 6.90 (dd, 1H), 6.80 (dd, 1H), 4.29 (d, 2H) |
| 206 | 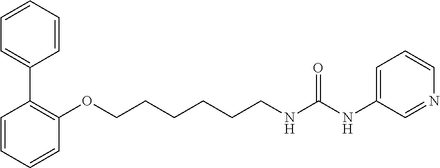 [SMILES: O=C(NCCCCCCOc1ccccc1-c1ccccc1)Nc1cccnc1] | 8.20-8.15 (m, 1H), 8.01-7.93 (m, 1H), 7.50 9d, 2H), 7.40 (t, 2H), 7.35-7.27 (m, 3H), 7.10 (d, 1H), 7.01 (t, 1H), 4.00-3.96 (m, 2H), 3.12-3.02 (m, 2H), 1.70-1.60 (m, 2H), 1.45-1.33 (m, 4H), 1.32-1.22 (m, 2H) |
| 207 | 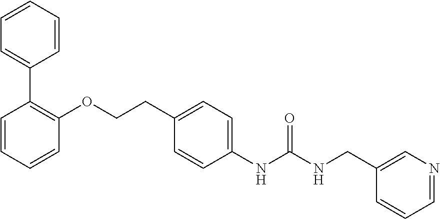 [SMILES: O=C(NCc1cccnc1)Nc1ccc(CCOc2ccccc2-c2ccccc2)cc1] | 8.56 (s, 1H), 8.52 (d, 1H), 8.45 (dd, 1H), 7.70 (dt, 1H), 7.39-7.23 (m, 11H), 7.07 (d, 2H), 7.01 (t, 1H), 6.65 (t, 1H), 4.31 (d, 2H), 4.15 (t, 2H), 2.87 (t, 2H) |
| 208 | 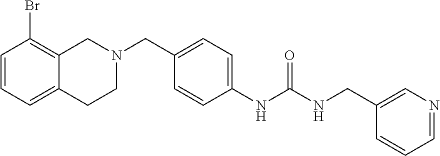 [SMILES: O=C(NCc1cccnc1)Nc1ccc(CN2CCc3cccc(Br)c3C2)cc1] | 8.62 (s, 1H), 8.53 (d, 1H), 8.45 (d, 1H), 7.71 (d, 1H), 7.42-7.33 (m, 4H), 7.21 (d, 2H), 7.17-7.06 (m, 2H), 6.70 (t, 1H), 4.32 (d, 2H), 3.61 (s, 2H), 3.45 (s, 2H), 2.82 (t, 2H), 2.64 (t, 2H) |
| 209 | 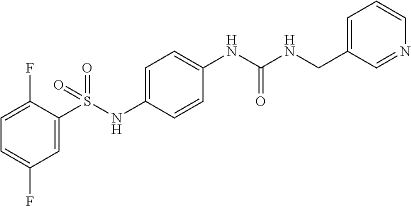 [SMILES: O=C(NCc1cccnc1)Nc1ccc(NS(=O)(=O)c2cc(F)ccc2F)cc1] | 10.40 (s, 1H), 8.60 (s, 1H), 8.49 (d, 1H), 8.45-8.43 (m, 1H), 7.70-7.65 (m, 1H), 7.60- 7.47 (m, 3H), 7.36-7.31 (m, 1H), 7.27 (d, 2H), 6.96 (d, 2H), 6.67 (t, 1H), 4.28 (d, 2H) |
| 210 | 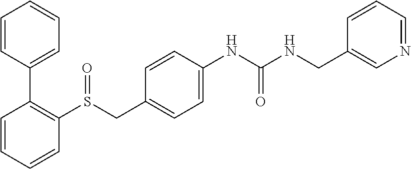 [SMILES: O=C(NCc1cccnc1)Nc1ccc(CS(=O)c2ccccc2-c2ccccc2)cc1] | 8.80 (s, 1H), 8.71 (d, 1H), 8.67 (d, 1H), 8.16 (d, 1H), 7.78-7.69 (m, 2H), 7.64-7.58 (m, 2H), 7.53-7.47 (m, 3H), 7.42-7.38 (m, 3H), 7.23 (d, 2H), 6.84 (t, 1H), 6.62 (d, 2H), 4.40 (d, 2H), 3.46 (AB, 2H) |
| 211 | 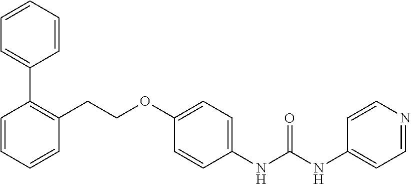 [SMILES: O=C(Nc1ccncc1)Nc1ccc(OCCc2ccccc2-c2ccccc2)cc1] | 9.05 (s, 1H), 8.69 (s, 1H), 8.33 (d, 2H), 7.54-7.19 (m, 12H), 6.70 (d, 2H), 3.99 (t, 2H), 2.98 (t, 2H) |
| 212 | 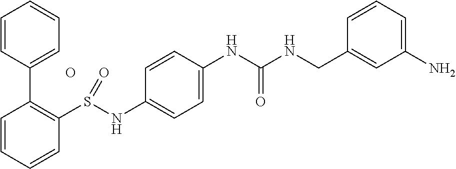 | 9.80 (s, 1H), 8.51 (s, 1H), 7.95 (dd, 1H), 7.61 (td, 1H), 7.55 (td, 1H), 7.41-7.36 (m, 4H), 7.29 (d, 1H), 7.26-7.20 (m, 5H), 6.88-6.75 (m, 4H), 6.55 (t, 1H), 4.20 (d, 2H) |
| 213 | 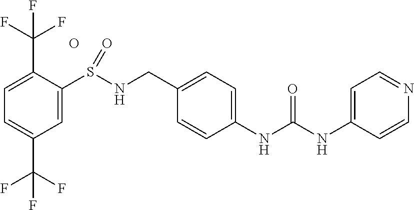 | 8.37 (dd, 2H), 8.16 (s, 1H), 8.04 (d, 1H), 7.98 (d, 1H), 7.60 (s, 1H), 7.42 (dd, 3H), 7.28 (dt, 2H), 7.10 (dt, 2H), 6.43 (s, 1H), 4.19 (d, 2H) |
| 214 | 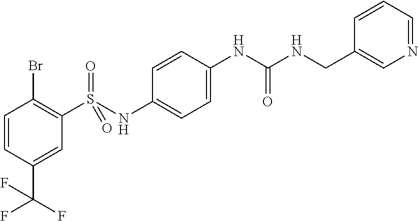 [SMILES: O=C(NCc1cccnc1)Nc1ccc(NS(=O)(=O)c2cc(C(F)(F)F)ccc2Br)cc1] | 10.53 (s, 1H), 8.60 (s, 1H), 8.49-8.48 (m, 1H), 8.45-8-42 (m, 1H), 8.15-8.13 (m, 1H), 8.12-8.07 (m, 1H), 7.70-7.64 (m, 1H), 7.36-7.30 (m, 1H), 7.26 (d, 2H), 6.96 (d, 2H), 6.66 (t, 1H), 4.27 (d, 2H) |
| 215 | 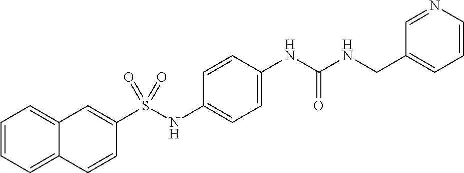 [SMILES: O=C(NCc1cccnc1)Nc1ccc(NS(=O)(=O)c2ccc3ccccc3c2)cc1] | 10.06 (s, 1H), 8.67-8.63 (m, 3H), 8.34 (d, 1H), 8.14-8.06 (m, 3H), 7.99 (d, 1H), 7.74- 7.61 (m, 4H), 7.20 (d, 2H), 6.94 (d, 2H), 6.76 (t, 1H), 4.35 (d, 2H) |
| 216 | 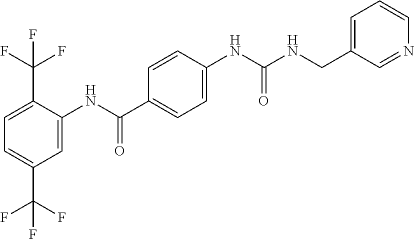 [SMILES: O=C(NCc1cccnc1)Nc1ccc(C(=O)Nc2cc(C(F)(F)F)ccc2C(F)(F)F)cc1] | 9.62 (s, 2H), 8.59 (s, 1H), 8.52 (d, 1H), 8.45 (dd, 1H), 7.70 (m, 1H), 7.64 (d, 1H), 7.42 (s, 2H), 7.41 (d, 2H), 7.39-7.26 (m, 8H), 7.08 (d, 2H), 6.67 (d, 1H), 4.31 (d, 2H), 3.98 (s, 2H) |
| 217 | 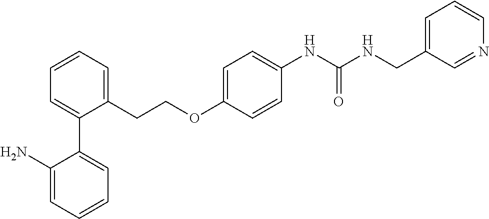 [SMILES: Nc1ccccc1-c1ccccc1CCOc1ccc(NC(=O)NCc2cccnc2)cc1] | 8.51 (d, 1H), 8.45 (dd, 1H), 8.39 (s, 1H), 7.69 (d, 1H), 7.46 (d, 1H), 7.37-7.29 (m, 3H), 7.18 (d, 2H), 7.13-7.08 (m, 2H), 6.91 (br d, 1H), 6.77 (br d, 1H), 6.65 (t, 1H), 6.59- 6.53 (m, 3H), 4.48 (br s, 2H), 4.29 (d, 2H), 3.93 (m, 2H), 2.87-2.81 (m, 2H) |
| 218 | 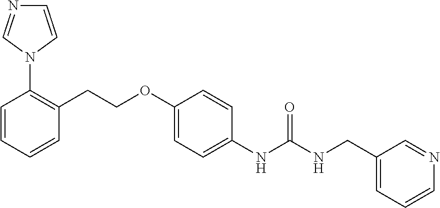 [SMILES: O=C(NCc1cccnc1)Nc1ccc(OCCc2ccccc2-n2ccnc2)cc1] | 8.49 (s, 1H), 8.04 (bs, 1H), 7.91 (d, 1H), 7.74-7.45 (m, 4H), 7.27 (d, 2H), 6.75 (d, 2H), 6.66 (t, 1H), 4.34 (d, 2H), 4.34 (t, 2H), 2.87 (t, 2H) |
| 219 | 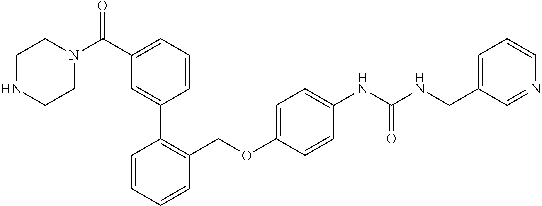 [SMILES: O=C(NCc1cccnc1)Nc1ccc(OCc2ccccc2-c2cccc(C(=O)N3CCNCC3)c2)cc1] | 8.88 (bs, 2H), 8.73-8.64 (m, 3H), 8.20-8.15 (m, 1H), 7.81- 7.75 (m, 1H), 7.62-7.59 (m, 1H), 7.54-7.51 (m, 2H), 7.50- 7.48 (m, 1H), 7.47-7.44 (m, 3H), 7.38-7.35 (m, 1H), 7.25 (d, 2H), 6.87 (bs, 1H), 6.76 (d, 2H), 4.90 (s, 2H), 4.40 (d, 2H), 3.81-3.50 (m, 4H) |
| 220 | 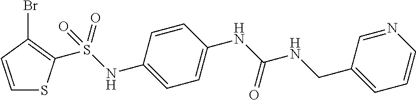 [SMILES: O=C(NCc1cccnc1)Nc1ccc(NS(=O)(=O)c2sccc2Br)cc1] | 10.46 (s, 1H), 8.73 (s, 1H), 8.64 (s, 1H), 8.60 (d, 1H), 8.02 (d, 1H), 7.91-7.87 (m, 1H), 7.66-7.61 (m, 1H), 7.28 (d, 2H), 7.24-7.21 (m, 1H), 6.99 (d, 2H), 6.78 (t, 1H), 4.36 (d, 2H) |
| 221 | 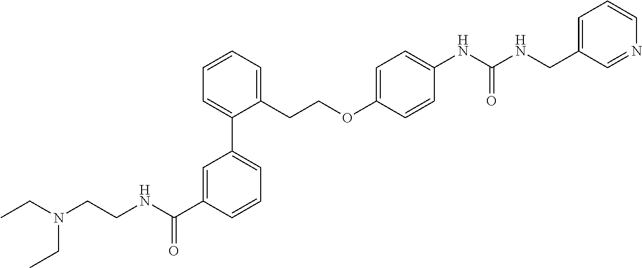 [SMILES: CCN(CC)CCNC(=O)c1cccc(-c2ccccc2CCOc2ccc(NC(=O)NCc3cccnc3)cc2)c1] | 9.74 (br s, 1H), 8.86 (br s, 1H), 8.52 (d, 2H), 8.44 (dd, 1H), 7.92 (d, 1H), 7.88 (s, 1H), 7.68 (dt , 1H), 7.60-7.57 (m, 2H), 7.49 (d, 1H), 7.41- 7.32 (m, 3H), 7.25-7.20 (m, 3H), 6.68 (t, 1H), 6.64 (d, 2H), 4.28 (d, 2H), 3.96 (t, 2H), 3.61 (m, 2H), 3.17 ( |
| 222 | 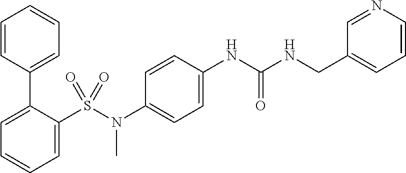 [SMILES: CN(c1ccc(NC(=O)NCc2cccnc2)cc1)S(=O)(=O)c1ccccc1-c1ccccc1] | 8.72 (s, 1H), 8.51 (d, 1H), 8.45 (dd, 1H), 7.82 (d, 1H), 7.70-7.67 (m, 2H), 7.55 (t, 1H), 7.39-7.33 (m, 5H), 7.29-7.26 (m, 4H), 6.79 (d, 2H), 6.74 (t, 1H), 4.29 (d, 2H), 2.70 (s, 3H) |
| 223 | 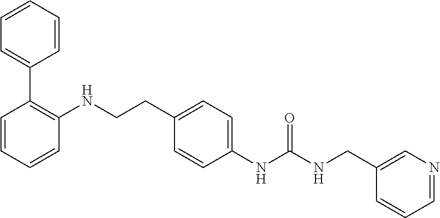 [SMILES: O=C(NCc1cccnc1)Nc1ccc(CCNc2ccccc2-c2ccccc2)cc1] | 8.76 (s, 1H), 8.75 (s, 1H), 8.71 (d, 1H), 8.25 (d, 1H), 7.83 (dd, 1H), 7.40 (dd, 2H), 7.33 (dd, 1H)7.29 (d, 2H), 7.23 (d, 2H), 7.18 (dd, 1H), 7.02 (d, 2H), 6.69 (d, 1H), 6.86 (dd, 1H), 6.74 (d, 1H), 6.68 (t, 1H), 4.43 (d, 2H), 3.24 (t, 2H), 2.71 (t, 2H) |
| 224 | 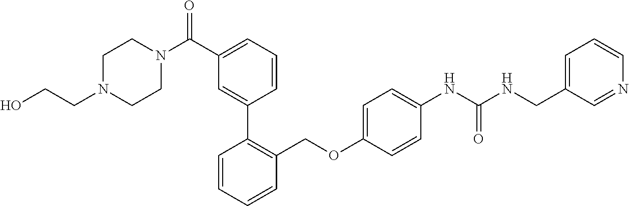 [SMILES: O=C(NCc1cccnc1)Nc1ccc(OCc2ccccc2-c2cccc(C(=O)N3CCN(CCO)CC3)c2)cc1] | 8.78-8.79 (m, 3H), 8.24 (bd, 1H), 7.83 (bt, 1H), 7.62-7.59 (m, 1H), 7.56-7.51 (m, 2H), 7.49-7.43 (m, 4H), 7.38-7.35 (m, 1H), 7.26 (d, 2H), 7.00 (bs, 1H), 6.76 (d, 2H), 4.90 (s, 2H), 4.41 (d, 2H), 3.71 (t, 2H), 3.60-3.07 (bm, 4H), 3.17 (t, 2H). |
| 225 | 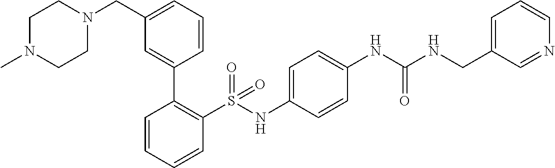 [SMILES: CN1CCN(Cc2cccc(-c3ccccc3S(=O)(=O)Nc3ccc(NC(=O)NCc4cccnc4)cc3)c2)CC1] | 9.78 (s, 1H), 8.76 (s, 1H), 8.69-8.61 (m, 2H), 8.08 (d, 1H), 7.99-7.94 (m, 1H), 7.74- 7.66 (m, 1H), 7.66-7.53 (m, 2H), 7.44-7.34 (m, 2H), 7.33- 7.15 (m, 5H), 6.89 (t, 1H), 6.81 (d, 2H), 4.37 (d, 2H), 3.82 (s, 2H), 3.48-2.54 (m, 11H) |
| 226 | 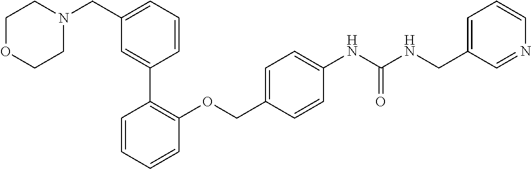 [SMILES: O=C(NCc1cccnc1)Nc1ccc(COc2ccccc2-c2cccc(CN3CCOCC3)c2)cc1] | 8.70 (s, 1H), 8.53-8.50 (m, 1H), 8.47-8.43 (m, 1H), 7.72- 7.68 (m, 1H), 7.46 (s, 1H), 7.40-7.16 (m, 11H), 7.06-7.00 (m, 1H), 6.72 (t, 1H), 5.01 (s, 2H), 4.31 (d, 2H) 3.56-3.51 (m, 4H), 3.45 (s, 2H), 2.36- 2.29 (m, 4H) |
| 227 | 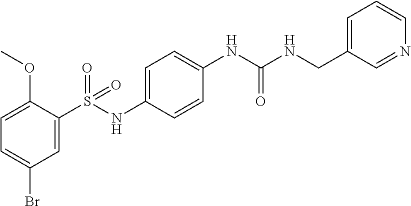 [SMILES: COc1ccc(Br)cc1S(=O)(=O)Nc1ccc(NC(=O)NCc2cccnc2)cc1] | 9.80 (s, 1H), 8.54 (s, 1H), 8.49 (d, 1H), 8.45-8.42 (m1H), 7.75-7.65 (m, 3H), 7.36-7.31 (m, 1H), 7.23 (d, 2H), 7.16 (d, 1H), 6.93 (d, 2H), 6.65 (t, 1H), 4.24 (d, 2H), 3.91 (s, 3H) |
| 228 | 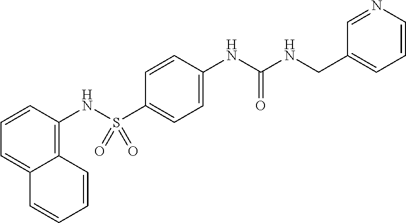 [SMILES: O=C(NCc1cccnc1)Nc1ccc(S(=O)(=O)Nc2cccc3ccccc23)cc1] | 10.06 (s, 1H), 9.28 (s, 1H), 8.68 (d, 1H), 8.64 (dd, 1H), 8.11-8.05 (m, 2H), 7.89 (dd, 1H), 7.76 (d, 1H), 7.71-7.68 (m, 1H), 7.55-7.37 (m, 5H), 7.13 (dd, 1H), 7.07 (t, 1H), 4.39 (d, 2H) |
| 229 | 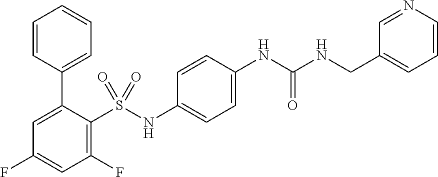 [SMILES: O=C(NCc1cccnc1)Nc1ccc(NS(=O)(=O)c2c(F)cc(F)cc2-c2ccccc2)cc1] | 10.17 (s, 1H), 9.72 (s, 1H), 8.75 (s, 1H), 8.64 (dd, 2H), 8.08 (d, 1H), 7.71-7.53 (m, 1H), 7.38-7.36 (m, 1H), 7.32-7.22 (m, 5H), 7.07-7.04 (m, 2H), 7.02-6.99 (m, 1H), 6.83 (d, 2H), 6.56 (dd, 1H), 6.07 (d, 1H), 4.37 (d, 2H) |
| 230 | 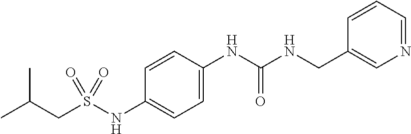 [SMILES: CC(C)CS(=O)(=O)Nc1ccc(NC(=O)NCc2cccnc2)cc1] | 9.47 (s, 1H), 8.67 (d, 1H), 8.58 (dd, 1H), 8.53 (d, 1H), 7.87 (bs, 1H), 7.51 (bs, 1H), 7.35 (d, 2H), 7.07 (d, 2H), 6.71 (t, 1H), 4.34 (d, 2H), 2.86 (d, 2H), 2.68 (t, 1H), 0.97 (d, 6H) |
| 231 | 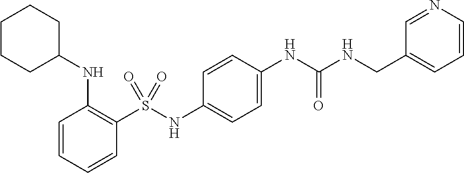 [SMILES: O=C(NCc1cccnc1)Nc1ccc(NS(=O)(=O)c2ccccc2NC2CCCCC2)cc1] | 9.77 (s, 1H), 8.64 (m, 3H), 8.04 (d, 1H), 7.92 (d, 1H), 7.68-7.49 (m, 5H), 7.39 (d, 1H), 7.21 (d, 2H), 7.19 (d, 1H), 6.82 (d, 2H), 6.75 (t, 1H), 4.35 (d, 2H). |
| 232 | 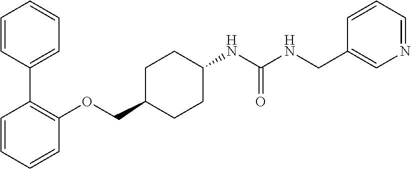 [SMILES: O=C(NCc1cccnc1)N[C@H]1CC[C@H](COc2ccccc2-c2ccccc2)CC1] | 8.45 (d, 1H), 8.43 (dd, 1H), 7.62 (dt, 1H), 7.52-7.26 (m, 8H), 7.08 (d, 1H), 7.02 (t, 1H), 6.27 (t, 1H), 5.85 (d, 1H), 4.21 (d, 2H), 3.80 (d, 2H), 3.30-3.21 (m, 1H), 1.86- 1.77 (m, 2H), 1.77-1.67 (m, 2H), 1.65-1.53 (m, 1H) |
| 233 | 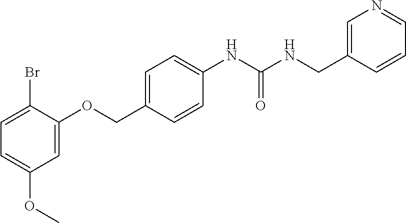 [SMILES: COc1ccc(Br)c(OCc2ccc(NC(=O)NCc3cccnc3)cc2)c1] | 8.71 (s, 1H), 8.52 (d, 1H), 8.45 (dd, 1H), 7.71 (dt, 1H), 7.45-7.31 (m, 6H), 6.76 (d, 1H), 6.74 (t, 1H), 6.50 (dd, 1H), 5.08 (s, 2H), 4.32 (d, 2H), 3.75 (s, 3H) |
| 234 | 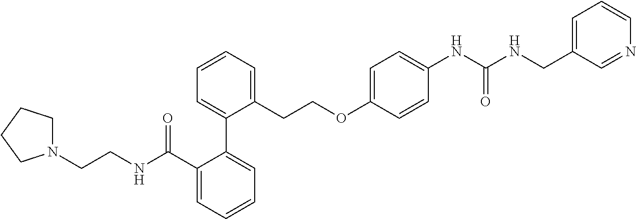 [SMILES: O=C(NCc1cccnc1)Nc1ccc(OCCc2ccccc2-c2ccccc2C(=O)NCCN2CCCC2)cc1] | 8.52-8.50 (m, 2H), 8.44 (dd, 1H), 7.68 (dt, 1H), 7.59 (dd, 1H), 7.54-7.46 (m, 2H), 7.40 (dd, 1H), 7.35 (dd, 1H), 7.33- 7.27 (m, 2H), 7.25-7.19 (m, 3H), 7.13 (dd, 1H), 6.68 (t, 1H), 6.60 (d, 2H), 4.28 (d, 2H), 4.02-4.39 (m, 2H), 3.42- 3.30 (m, 6H), 3.07-2. |
| 235 | 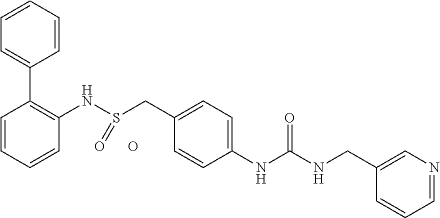 | 8.79 (s, 1H), 8.69 (s, 1H), 8.52 (d, 1H), 8.47-8.44 (m, 1H), 7.73-7.68 (m, 1H), 7.48- 7.41 (m, 4H), 7.40-7.25 (m, 8H), 7.09 (d, 2H), 6.71 (t, 1H), 4.31 (d, 2H), 4.06 (s, 2H) |
| 236 | 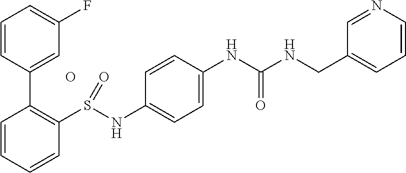 | 9.85 (br s, 1H), 8.70 (s, 1H), 8.65-8.61 (m, 2H), 8.06-8.04 (m, 1H), 7.96 (dd, 1H), 7.68- 7.56 (m, 3H), 7.44-7.37 (m, 1H), 7.30 (dd, 1H), 7.22 (d, 2H), 7.03-6.98 (m, 2H), 6.80 (d, 2H), 4.35 (d, 2H) |
| 237 | 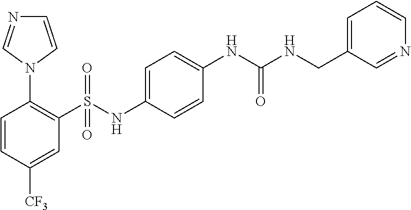 [SMILES: O=C(NCc1cccnc1)Nc1ccc(NS(=O)(=O)c2cc(C(F)(F)F)ccc2-n2ccnc2)cc1] | 10.51 (s, 1H), 9.11 (bs, 1H), 8.83 (s, 1H), 8.34 (dd, 1H), 8.23 (d, 1H), 8.03 (dd, 2H), 7.83-7.62 (bs, 3H), 7.32 (d, 2H), 6.90 (d, 2H), 6.86 (t, 1H), 4.37 (d, 2H) |
| 238 | 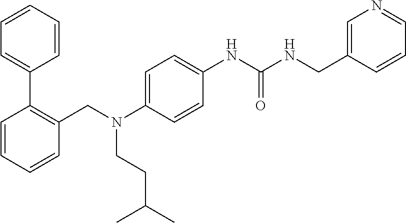 [SMILES: CC(C)CCN(Cc1ccccc1-c1ccccc1)c1ccc(NC(=O)NCc2cccnc2)cc1] | 8.49 (br d, 1H), 8.43 (dd, 1H), 8.13 (s, 1H), 7.67 (dt, 1H), 7.50-7.39 (m, 5H), 7.34 (ddd, 1H), 7.30-7.27 (m, 2H), 7.23-7.20 (m, 2H), 7.07 (d, 2H), 6.50 (t, 1H), 6.41 (d, 1H), 4.32 (s, 2H), 4.26 (d, 2H), 3.24 (t, 2H), 1.55-1.45 (m, 1H), 1.30 (q, 2H), 0. |
| 239 | 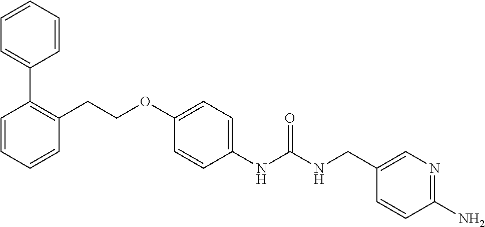 [SMILES: Nc1ccc(CNC(=O)Nc2ccc(OCCc3ccccc3-c3ccccc3)cc2)cn1] | 8.51 (s, 1H), 7.90 (s, 1H), 7.87 (d, 1H), 7.81 (s, 1H), 7.49-7.43 (m, 3H), 7.42-7.33 (m, 4H), 7.30 (dd, 1H), 7.24- 7.18 (m, 3H), 6.95 (d, 1H), 6.67-6.59 (m, 3H), 4.14 (d, 2H), 3.95 (dd, 2H), 2.96 (dd, 2H) |
| 240 | 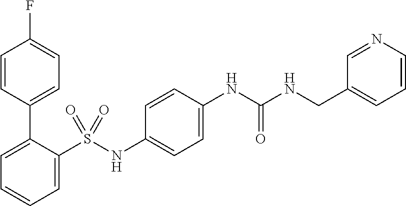 [SMILES: O=C(NCc1cccnc1)Nc1ccc(NS(=O)(=O)c2ccccc2-c2ccc(F)cc2)cc1] | 9.74 (s, 1H), 8.59 (br s, 1H), 8.53 (d, 1H), 8.48 (dd, 1H), 7.96 (d, 1H), 7.77 (dt, 1H), 7.65-7.52 (m, 2H), 7.42 (dd, 1H), 7.28 (dd, 1H), 7.24-7.19 (m, 6H), 6.79 (d, 2H), 6.71 (t, 1H), 4.30 (d, 2H) |
| 241 | 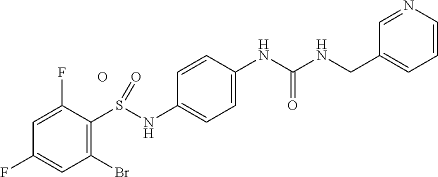 | 10.5 (s, 1H), 8.6 (s, 1H), 8.49 (d, 1H), 8.44 (dd, 1H), 7.73- 7.66 (m, 2H), 7.61-7.55 (m, 1H), 7.34 (ddd, 1H), 7.28 (d, 2H), 6.97 (d, 2H), 6.68 (t, 1H), 4.28 (d, 2H) |
| 242 | 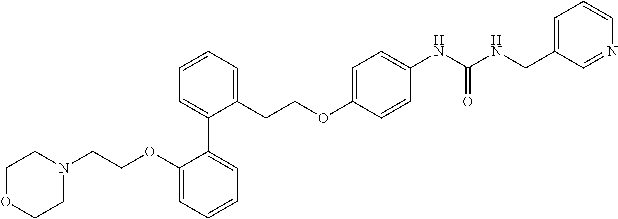 [SMILES: O=C(NCc1cccnc1)Nc1ccc(OCCc2ccccc2-c2ccccc2OCCN2CCOCC2)cc1] | 8.51 (d, 1H), 8.45 (dd, 1H), 8.39 (s, 1H), 7.70-7.67 (dt, 1H), 7.41-7.23 (m, 5H), 7.19 (d, 2H), 7.16 (dd, 1H), 7.12- 7.08 (m, 2H), 7.04 (td, 1H), 6.60-6.54 (m, 3H), 4.29 (d, 2H), 4.02 (td, 2H), 3.91-3.86 (m, 2H), 3.41-3.37 (m, 4H), 2.84-2.79 (m, 2H), 2.4 |
| 243 | 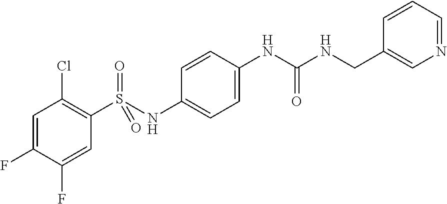 [SMILES: O=C(NCc1cccnc1)Nc1ccc(NS(=O)(=O)c2cc(F)c(F)cc2Cl)cc1] | 10.45 (s, 1H), 8.59 (s, 1H), 8.49 (s, 1H), 8.44 (d, 2H), 8.05-7.92 (m, 2H), 7.67 (d, 1H), 7.37-7.31 (m, 1H), 7.27 (d, 2H), 6.97 (d, 2H), 6.67 (t, 2H), 4.28 (d, 2H) |
| 244 | 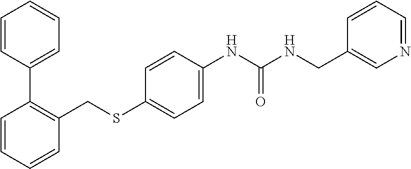 [SMILES: O=C(NCc1cccnc1)Nc1ccc(SCc2ccccc2-c2ccccc2)cc1] | 8.78 (s, 1H), 8.63 (d, 1H), 8.58 (d, 1H), 7.97 (d, 1H), 7.59 (bs, 1H), 7.48-7.27 (m, 10H), 7.23-7.19 (m, 1H), 7.08 (d, 2H), 6.79 (t, 1H), 4.36 (d, 2H), 4.01 (s, 2H) |
| 245 | 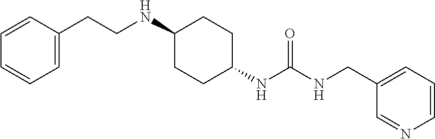 [SMILES: O=C(NCc1cccnc1)N[C@H]1CC[C@H](NCCc2ccccc2)CC1] | 8.73 (s, 2H), 8.47 (d, 1H), 7.99 (dd, 1H), 7.24-7.36 (m, 5H), 4.49 (s, 2H), 3.48 (tt, 1H), 2.35 (t, 2H), 3.08 (tt, 1H), 2.97 (t, 2H), 2.16 (d, 2H), 2.03 (d, 2H), 1.47 (q, 2H), 1.32 (q, 2H) |
| 246 | 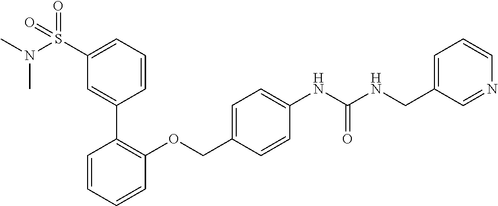 [SMILES: CN(C)S(=O)(=O)c1cccc(-c2ccccc2OCc2ccc(NC(=O)NCc3cccnc3)cc2)c1] | 8.90 (s, 1H), 8.73 (s, 1H), 8.69 (s, 1H), 8.20 (d, 1H), 7.89 (s, 1H), 7.85-7.75 (m, 2H), 7.75-7.64 (m, 2H), 7.43- 7.33 (m, 4H), 7.29-7.23 (m, 3H), 7.11-7.05 (m, 1H), 6.92 (t, 1H), 5.03 (s, 2H), 4.42 (d, 2H), 2.49 (s, 6H) |
| 247 | 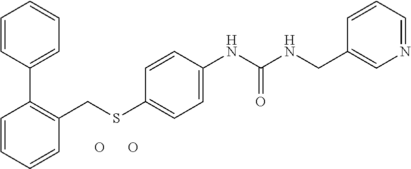 | 9.40 (s, 1H), 8.72 (d, 1H), 8.66 (dd, 1H), 8.14 (d, 1H), 7.73 (dd, 1H), 7.49 (d, 2H), 7.44-7.32 (m, 6H), 7.27 (d, 2H), 7.20-7.14 (m, 1H), 7.12 (t, 1H). 7.04 (dd, 2H), 4.47 (s, 2H), 4.44 (d, 2H) |
| 248 | 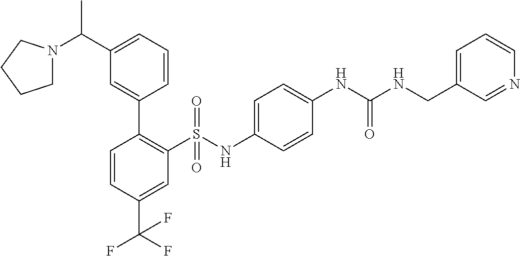 [SMILES: CC(c1cccc(-c2ccc(C(F)(F)F)cc2S(=O)(=O)Nc2ccc(NC(=O)NCc3cccnc3)cc2)c1)N1CCCC1] | 10.09 (s, 1H), 10.00 (bs, 1H), 8.85 (s, 1H), 8.70-8.63 (m, 2H), 8.29 (s, 1H), 8.13-8.03 (m, 2H), 7.74-7.69 (m, 1H), 7.63-7.48 (m, 3H), 7.38 (s, 1H), 7.30-7.24 (m, 3H), 6.95 (t, 1H), 6.82 (d, 2H), 4.43 (t, 1H), 4.38 (d, 2H), 3.73-3.62 (m, 1H), 3.22-2.84 ( |
| 249 | 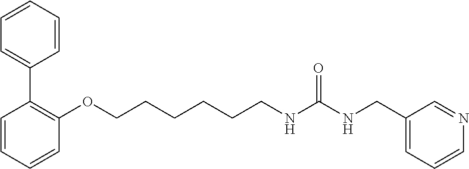 [SMILES: O=C(NCCCCCCOc1ccccc1-c1ccccc1)NCc1cccnc1] | 8.59-8.53 (m, 2H), 7.91 (d, 1H, 7.59-7.54 (m, 1H), 7.49 (d, 2H), 7.40 (t, 2H), 7.35- 7.26 (m, 3H), 7.09 (d, 1H), 7.01 (t, 1H), 6.44 (t, 1H), 6.07 (t, 1H), 4.26 (d, 2H), 3.96 (t, 2H), 3.01-2.93 (m, 2H), 1.68-1.58 (m, 2H), 1.39- 1.30 (m, 4H), 1.30-1.22 (m, 2H |
| 250 | 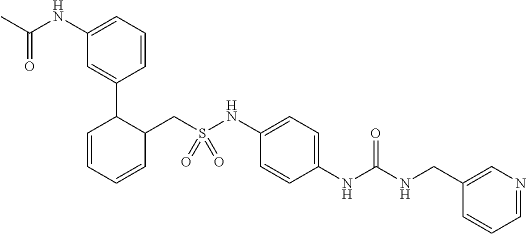 [SMILES: CC(=O)Nc1cccc(C2C=CC=CC2CS(=O)(=O)Nc2ccc(NC(=O)NCc3cccnc3)cc2)c1] | 9.98 (s, 1H), 9.64 (s, 1H), 8.67 (s, 2H), 8.62 (d, 1H), 8.07 (d, 1H), 7.67 (dd, 1H), 7.59 (d, 1H), 7.51-7.47 (m, 1H), 7.47-7.44 (m, 1H), 7.44- 7.39 (m, 2H), 7.29-7.22 (m, 4H), 6.98 (d, 1H), 6.87 (d, 2H), 6.77 (dd, 1H), 4.39 (d, 2H), 4.35 (s, 2H), 2.04 (s, |
| 251 | 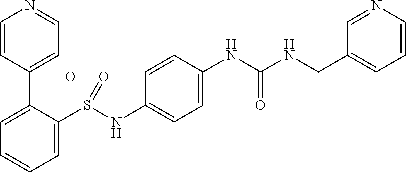 | 9.94 (s, 1H), 8.78 (s, 1H), 8.74 (d, 2H), 8.69-8.65 (m, 2H), 8.14 (dt, 1H), 8.00 (dd, 1H), 7.76-7.66 (m, 3H), 7.48 (d, 2H), 7.34 (dd, 1H), 7.24 (d, 2H), 6.87 (t, 1H), 6.80 (d, 2H), 4.38 (d, 2H) |
| 252 | 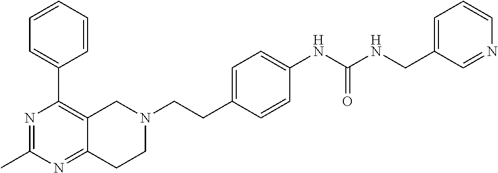 [SMILES: Cc1nc2c(c(-c3ccccc3)n1)CN(CCc1ccc(NC(=O)NCc3cccnc3)cc1)CC2] | 8.55-8.41 (m, 3H), 7.69 (d, 1H), 7.61-7.45 (m, 5H), 7.38- 7.31 (m, 1H), 7.25 (d, 2H), 7.03 (d, 2H), 6.64 (t, 1H), 4.30 (d, 2H), 3.56 (s, 2H), 2.95-2.81 (m, 4H), 2.69-2.54 (m, 7H) |
| 253 | 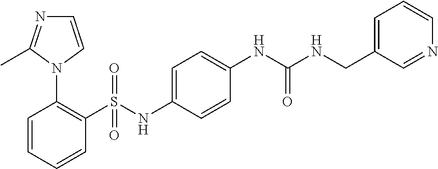 [SMILES: Cc1nccn1-c1ccccc1S(=O)(=O)Nc1ccc(NC(=O)NCc2cccnc2)cc1] | 10.46 (s, 1H), 8.74 (s, 1H), 8.06 (dd, 1H), 7.94-7.85 (m, 4H), 7.76 (d, 1H), 7.75 (d, 1H), 7.61 (d, 1H), 7.55 (d, 1H), 7.30 (d, 2H), 6.92 (d, 2H), 6.77 (t, 1H), 4.33 (d, 2H), 2.20 (s, 3H) |
| 254 | 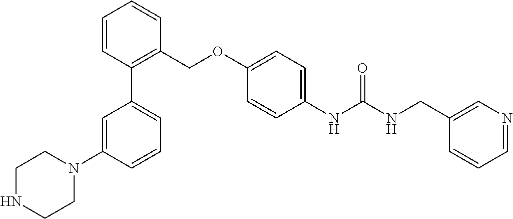 [SMILES: O=C(NCc1cccnc1)Nc1ccc(OCc2ccccc2-c2cccc(N3CCNCC3)c2)cc1] | 8.74 (bs, 1H), 8.66-8.59 (m, 3H), 8.02 (d, 1H), 7.67-7.56 (m, 2H), 7.46-7.40 (m, 2H), 7.36-7.25 (m, 6H), 7.03-6.97 (m, 2H), 6.90 (d, 1H), 6.85- 6.76 (m, 3H), 4.86 (s, 2H), 4.36 (d, 2H), 3.33-3.23 (m, 4H), 3.19-3.10 (m, 4H) |
| 255 | 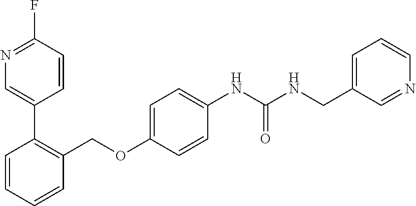 [SMILES: O=C(NCc1cccnc1)Nc1ccc(OCc2ccccc2-c2ccc(F)nc2)cc1] | 8.62-8.73 (m, 2 H) 8.57 (s, 1 H) 8.27 (d, 1 H) 8.11-8.16 (m, 1 H) 8.05 (td, 1 H) 7.74 (dd, 1 H) 7.60-7.65 (m, 1 H) 7.49 (ddd, 2 H) 7.35-7.40 (m, 1 H) 7.21-7.30 (m, 3 H) 6.70-6.80 (m, 3 H) 4.88 (s, 2 H) 4.38 (d, 2 H). |
| 256 | 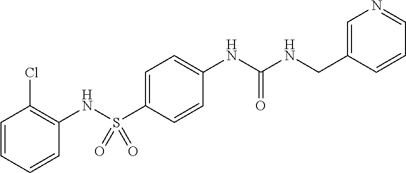 [SMILES: O=C(NCc1cccnc1)Nc1ccc(S(=O)(=O)Nc2ccccc2Cl)cc1] | 9.78 (s, 1H), 9.29 (s, 1H), 8.64 (d, 1H), 8.58 (d, 1H), 7.98 (br d, 1H), 7.61-7.52 (m, 4H), 7.39 (dd, 1H), 7.29-7.23 (m, 2H), 7.20-7.16 (m, 1H), 7.06 (t, 1H), 4.38 (d, 2H) |
| 257 | 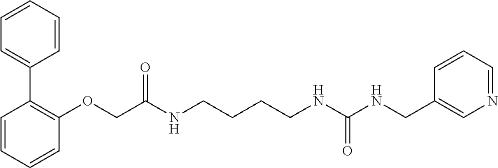 [SMILES: O=C(COc1ccccc1-c1ccccc1)NCCCCNC(=O)NCc1cccnc1] | 8.59-8.51 (m, 3H), 7.87 (bs, 2H), 7.67 (t, 2H), 7.59 (d, 2H), 7.54 (bs, 1H), 7.43 (t, 3H), 7.37-7.30 (m, 4H), 7.07 (t, 2H), 7.00 (d, 2H), 4.48 (s, 2H), 4.27 (d, 2H), 3.13-3.06 (m, 2H), 3.02-2.96 (m, 2H), 1.42-1.26 (m, 2H) |
| 258 | 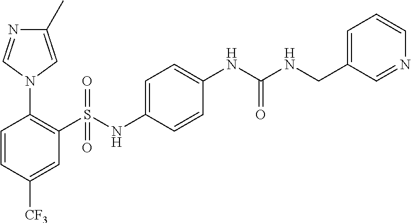 [SMILES: Cc1cn(-c2ccc(C(F)(F)F)cc2S(=O)(=O)Nc2ccc(NC(=O)NCc3cccnc3)cc2)cn1] | 10.48 (s, 1H), 9.03 (s, 1H), 8.80 (s, 2H), 8.57 (d, 2H), 8.33 (dd, 1H), 8.26 (s, 1H), 7.96 (d, 1H), 7.90 (d, 1H), 7.57-7.52 (m, 1H), 7.32 (d, 2H), 7.20 (s, 1H), 6.87 (d, 2H), 6.83 (t, 1H), 4.34 (d, 2H), 2.28 (s, 3H) |
| 259 | 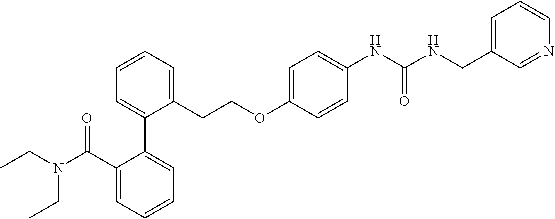 [SMILES: CCN(CC)C(=O)c1ccccc1-c1ccccc1CCOc1ccc(NC(=O)NCc2cccnc2)cc1] | 8.51 (d, 1H), 8.44 (dd, 1H), 8.39 (s, 1H), 7.69 (dt, 1H), 7.48-7.44 (m, 2H), 7.37-7.29 (m, 4H), 7.21 (t, 3H), 7.12 (br s, 1H), 6.59 (t, 1H), 4.29 (d, 2H), 2.94-2.81 (m, 4H), 0.94 (m, 3H), 0.58 (t, 3H) |
| 260 | 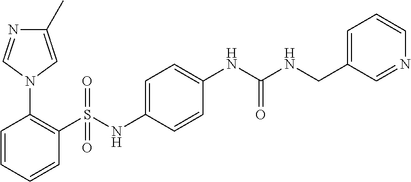 [SMILES: Cc1cn(-c2ccccc2S(=O)(=O)Nc2ccc(NC(=O)NCc3cccnc3)cc2)cn1] | 10.31 (s, 1H), 9.21 (s, 1H), 8.81 (s, 1H), 8.65 (bs, 1H), 8.03 (s, 1H), 8.00 (dd, 1H), 7.90-7.80 (m, 2H), 7.75-7.51 (m, 4H), 7.30 (d, 2H), 7.27 (s, 1H), 6.99 (dd, 1H), 6.87 (d, 2H), 4.27 (d, 2H), 2.30 (s, 3H) |
| 261 | 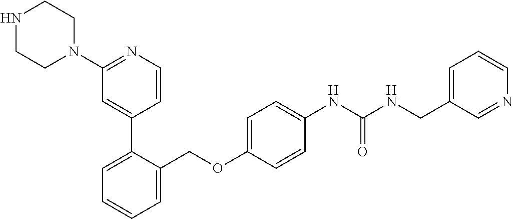 [SMILES: O=C(NCc1cccnc1)Nc1ccc(OCc2ccccc2-c2ccnc(N3CCNCC3)c2)cc1] | 8.92 (bs, 2H), 8.84-8.71 (m, 2H), 8.27 (d, 1H), 8.18 (d, 1H), 7.89-7.94 (m, 1H), 7.63 (dd, 1H), 7.48 (dd, 2H), 7.38 (dd, 1H), 7.29 (d, 2H), 7.03 (t, 1H), 6.97 (s, 1H), 6.84- 6.79 (m, 3H), 4.91 (s, 2H), 4.43 (d, 2H), 3.71-3.67 (m, 4H), 3.15-3.09 (m, 4H). |
| 262 | 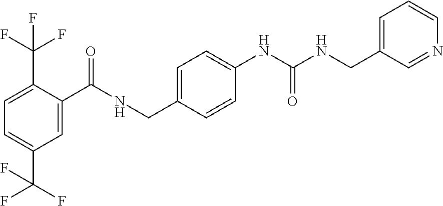 [SMILES: O=C(NCc1cccnc1)Nc1ccc(CNC(=O)c2cc(C(F)(F)F)ccc2C(F)(F)F)cc1] | 9.14 (t, 1H), 8.86 (s, 1H), 8.79-8.69 (m, 2H), 8.25 (d, 1H), 8.05 (d, 2H), 7.92 (s, 1H), 7.86-7.81 (m, 1H), 7.38 (d, 2H), 7.21 (d, 2H), 6.94 (t, 1H), 4.43 (d, 2H), 4.38 (d, 2H) |
| 263 | 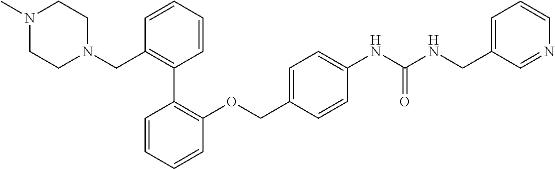 [SMILES: CN1CCN(Cc2ccccc2-c2ccccc2OCc2ccc(NC(=O)NCc3cccnc3)cc2)CC1] | 8.98 (s, 1H), 8.74-8.67 (m, 2H), 8.18 (d, 1H), 7.81-7.75 (m, 1H), 7.54-7.48 (m, 1H), 7.41-7.30 (m, 4H), 7.21-6.99 (m, 8H), 5.00-4.89 (m, 2H), 4.44-4.36 (m, 2H), 3.63-3.46 (m, 1H), 3.42-2.57 (m, 10H) |
| 264 | 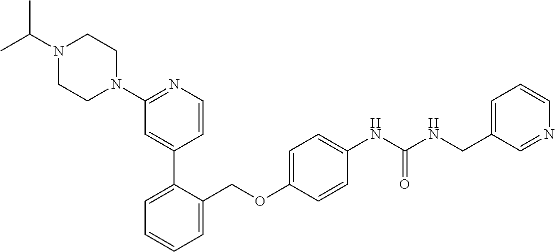 [SMILES: CC(C)N1CCN(c2cc(-c3ccccc3COc3ccc(NC(=O)NCc4cccnc4)cc3)ccn2)CC1] | 9.58 (bs, 1H), 8.79-8.72 (m, 3H), 8.27 (d, 1H), 8.19 (d, 1H), 7.86 (dd, 1H), 7.65-7.61 (m, 1H), 7.51-7.47 (m, 2H), 7.40-7.37 (m, 1H), 7.30 (d, 2H), 6.97 (s, 2H), 6.85-6.80 (m, 3H), 4.90 (s, 2H), 4.43- 4.33 (m, 4H), 3.50-3.43 (m, 1H), 3.42-3.35 (m, 2H), 3.1 |
| 265 | 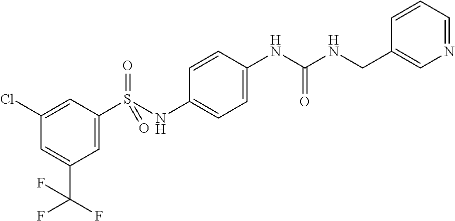 [SMILES: O=C(NCc1cccnc1)Nc1ccc(NS(=O)(=O)c2cc(Cl)cc(C(F)(F)F)c2)cc1] | 10.18 (s, 1H), 8.84 (s, 1H), 8.71-8.64 (m, 2H), 8.16 (d, 1H), 8.04 (s, 1H), 7.94-7.86 (m, 2H), 7.79-7.73 (m, 1H), 7.30 (d, 2H), 6.92 (d, 2H), 6.87 (t, 1H), 4.39 (d, 2H) |
| 266 | 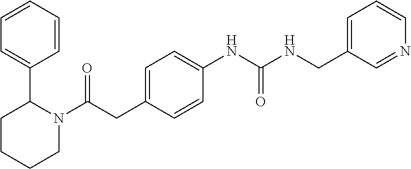 [SMILES: O=C(NCc1cccnc1)Nc1ccc(CC(=O)N2CCCCC2c2ccccc2)cc1] | 8.75 (bs, 1H), 8.70 (s, 1H), 8.65 (s, 1H), 8.13 (d, 1H), 7.76-7.70 (m, 1H), 7.45-7.03 (m, 8H), 6.86-6.78 (m, 1H), 4.40 (d, 2H), 3.90-3.64 (m, 3H), 2.87-2.70 (m, 2H), 2.39- 2.32 (m, 2H), 1.73-1.12 (m, 4H) |
| 267 | 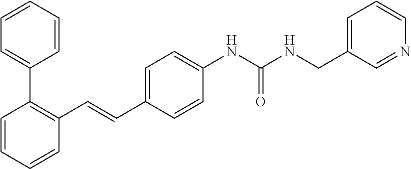 [SMILES: O=C(NCc1cccnc1)Nc1ccc(/C=C/c2ccccc2-c2ccccc2)cc1] | 8.77 (s, 1H), 8.52 (d, 1H), 8.46 (dd, 1H), 7.82 (dd, 1H), 7.70 (dt, 1H), 7.52-7.28 (m, 11H), 7.24 (d, 2H), 7.11 (d, 1H), 6.89 (d, 1H), 6.76 (t, 1H), 4.31 (d, 2H) |
| 268 | 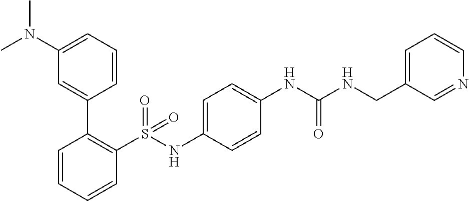 [SMILES: CN(C)c1cccc(-c2ccccc2S(=O)(=O)Nc2ccc(NC(=O)NCc3cccnc3)cc2)c1] | 9.70 (s, 1H), 8.78-8.71 (m, 3H), 8.29 (d, 1H), 7.97-7.93 (m, 1H), 7.90-7.85 (ml H), 7.64-7.48 (m, 2H), 7.32-7.28 (m, 1H), 7.26-7.18 (m, 3H), 6.90-6.80 (m, 3H), 6.71 (s, 1H), 6.64 (d, 1H), 4.41 (d, 2H), 2.93 (s, 6H) |
| 269 | 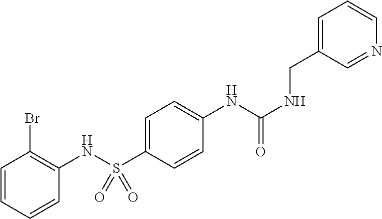 [SMILES: O=C(NCc1cccnc1)Nc1ccc(S(=O)(=O)Nc2ccccc2Br)cc1] | 9.64 (d, 1H), 8.53 (d, 1H), 8.46 (d, 1H), 7.71 (d, 1H), 7.60-7.52 (m, 5H), 7.36 (dd, 1H), 7.31 (dd, 1H), 7.26-7.20 (bs, 1H), 7.16 (d, 1H), 7.12 (dd, 2H), 4.33 (d, 2H) |
| 270 | 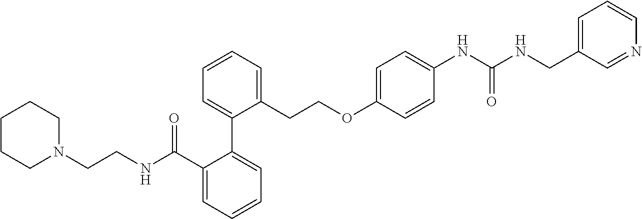 [SMILES: O=C(NCc1cccnc1)Nc1ccc(OCCc2ccccc2-c2ccccc2C(=O)NCCN2CCCCC2)cc1] | 9.13 (br s, 1H), 8.67 (d, 1H), 8.65 (dd, 1H), 8.58 (s, 1H), 8.46 (t, 1H), 8.09 (dt, 1H), 7.71 (dd, 1H), 7.56-7.47 (m, 2H), 7.41 (dd, 1H), 7.33-7.29 (m, 2H), 7.26-7.19 (m, 3H), 7.12 (dd, 1H), 6.78 (t, 1H), 6.60 (d, 2H), 4.37 (d, 2H), 4.02-3.89 (m, 4H), 3 |
| 271 | 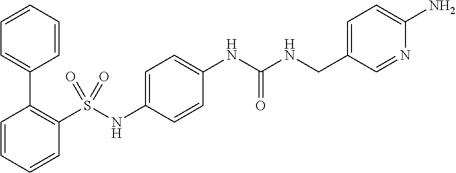 [SMILES: Nc1ccc(CNC(=O)Nc2ccc(NS(=O)(=O)c3ccccc3-c3ccccc3)cc2)cn1] | 9.77 (s, 1H), 8.44 (s, 1H), 7.95 (d, 1H), 7.80 (s, 1H), 7.62 (dd, 1H), 7.55 (dd, 1H), 7.45 (d, 1H), 7.41-7.36 (m, 3H), 7.28 (d, 1H), 7.26-7.18 (m, 4H), 6.81 (d, 2H), 6.54 (d, 1H), 6.44 (dd, 1H), 4.07 (d, 2H) |
| 272 | 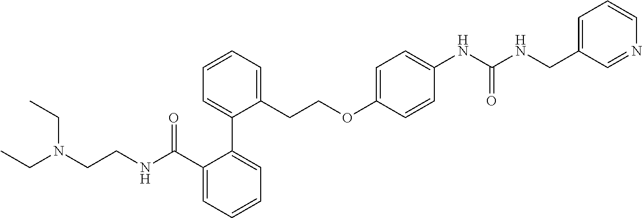 [SMILES: CCN(CC)CCNC(=O)c1ccccc1-c1ccccc1CCOc1ccc(NC(=O)NCc2cccnc2)cc1] | 9.17 (br s, 1H), 8.65 (d, 1H), 8.62 (dd, 1H), 8.57 (s, 1H), 8.42 (t, 1H), 8.04 (dt, 1H), 7.68-7.65 (m, 1H), 7.57-7.47 (m, 3H), 7.41 (dd, 1H), 7.33- 7.28 (m, 2H), 7.25-7.19 (m, 3H), 7.11 (dd, 1H), 6.77 (t, 1H), 6.61 (d, 2H), 4.36 (d, 2H), 4.01-4.89 (m, 2H |
| 273 | 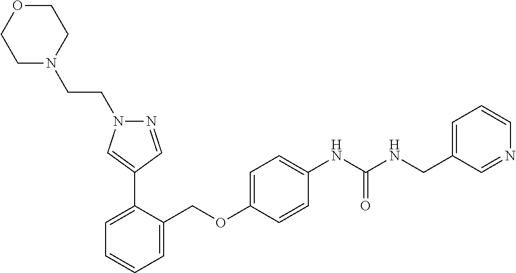 [SMILES: O=C(NCc1cccnc1)Nc1ccc(OCc2ccccc2-c2cnn(CCN3CCOCC3)c2)cc1] | 8.78 (s, 1H), 8.75-8.69 (m, 1H), 8.82 (bd, 1H), 8.06 (m, 1H), 7.83-7.79 (m, 1H), 7.75 (s, 1H), 7.56 (d, 1H), 7.49- 7.46 (m, 1H), 7.41 (t, 1H), 7.35-7.31 (m, 3H), 6.97 (t, 1H), 6.90 (d, 2H), 4.99 (s, 2H), 4.57 (t, 2H), 4.42 (d, 2H), 3.79 (m, 4H), 3.61 (t, 2 |
| 274 | 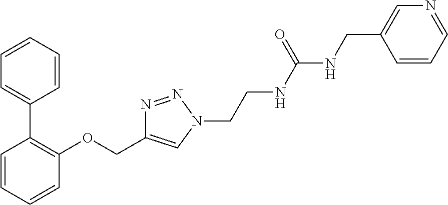 [SMILES: O=C(NCCn1cc(COc2ccccc2-c2ccccc2)nn1)NCc1cccnc1] | 8.41 (br s 1H), 8.40 (br s, 1H), 7.53 (d, 1H), 7.47 (d, 2H), 7.36-7.26 (m, 6H), 7.15 (dd, 1H), 7.08-7.03 (m, 2H), 5.53 (t, 1H), 5.39 (t, 1H), 5.30 (s, 1H), 5.04 (s, 2H), 4.34 (t, 2H), 4.27 (d, 2H), 3.61 (q, 2H) |
| 275 | 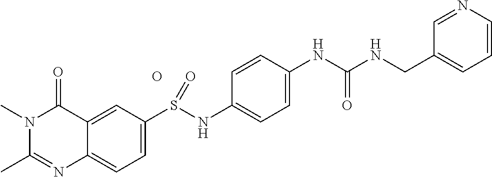 | ()10.07 (s, 1H), 8.73 (s, 1H), 8.66 (s, 1H), 8.64 (d, 1H), 8.37 (d, 1H), 8.09 (d, 1H), 7.94 (dd, 1H), 7.71 (dd, 1H), 7.68 (d, 1H), 7.23 (d, 2H), 6.90 (d, 2H), 6.79 (t, 1H), 4.35 (d, 2H), 2.57 (s, 3H), 2.51 (s, 3H) |
| 276 | 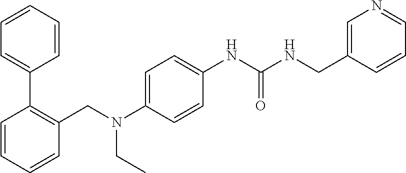 [SMILES: CCN(Cc1ccccc1-c1ccccc1)c1ccc(NC(=O)NCc2cccnc2)cc1] | 8.49 (br d, 1H), 8.43 (dd, 1H), 8.12 (s, 1H), 7.67 (dt, 1H), 7.50-7.37 (m, 6H), 7.36- 7.32 (m, 1H), 7.30-7.27 (m, 2H), 7.25-7.21 (m, 2H), 7.07 (d, 2H)6.48 (t, 1H), 6.42 (d, 2H), 4.31 (s, 2H), 4.26 (d, 2H), 0.99 (t, 3H) |
| 277 | 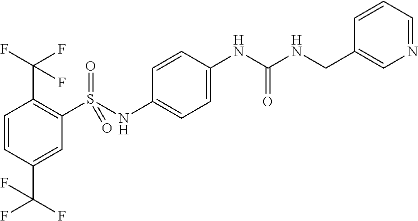 [SMILES: O=C(NCc1cccnc1)Nc1ccc(NS(=O)(=O)c2cc(C(F)(F)F)ccc2C(F)(F)F)cc1] | 10.57 (s, 1H), 8.79 (s, 1H), 8.69-8.60 (m, 2H), 8.30 (s, 1H), 8.25-8.20 (m, 2H), 8.08 (d, 1H), 7.73-7.66 (m, 1H), 7.30 (d, 2H), 6.97 (d, 2H), 6.83 (t, 1H), 4.36 (d, 2H) |
| 278 | 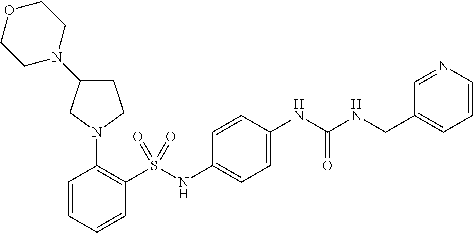 [SMILES: O=C(NCc1cccnc1)Nc1ccc(NS(=O)(=O)c2ccccc2N2CCC(N3CCOCC3)C2)cc1] | 9.61 (s, 1H), 8.88 (s, 1H), 8.68 (br s, 2H), 8.15 (d, 1H), 7.76 (d, 1H), 7.55 (t, 1H), 7.44 (d, 1H), 7.22 (d, 2H), 7.19 (t, 1H), 7.03 (t, 1H), 6.91 (d, 2H), 4.37 (d, 2H), 3.96-3.05 (m, 13H), 2.40 (m, 1H), 2.21 (m, 1H). |
| 279 | 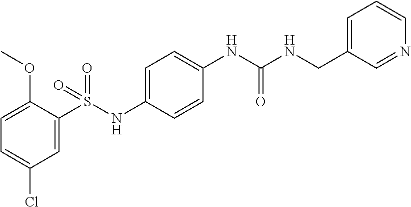 [SMILES: COc1ccc(Cl)cc1S(=O)(=O)Nc1ccc(NC(=O)NCc2cccnc2)cc1] | 10.40 (s, 1H), 8.60 (s, 1H), 8.50-8.48 (m, 1H), 8.46-8.42 (m, 1H), 7.71-7.64 (m, 1H), 7.63-7.59 (m, 1H), 7.58-7.56 (m, 1H), 7.36-7.31 (m, 1H), 7.25-7.19 (m, 3H), 6.93 (d, 2H), 6.65 (t, 1H), 4.27 (d, 2H), 3.92 (s, 3H) |
| 280 | 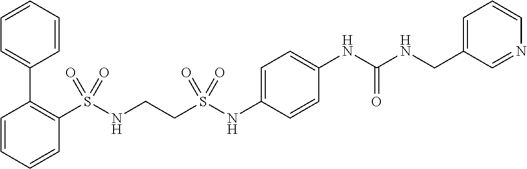 [SMILES: O=C(NCc1cccnc1)Nc1ccc(NS(=O)(=O)CCNS(=O)(=O)c2ccccc2-c2ccccc2)cc1] | 9.50 (s, 1H), 8.83 (s, 1H), 8.69 (s, 1H), 8.64 (d, 1 h), 8.09 (d, 1H), 7.89 (d, 1H), 7.72-7.63 (m, 2H), 7.58 (dd, 1H), 7.40-7.28 (m, 9H), 7.00 (d, 2H), 6.68 (dd, 1H), 4.40 (d, 2H), 3.00 (m, 4H) |
| 281 | 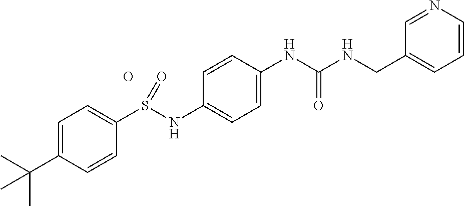 | 9.96 (s, 1H), 8.72 (s, 1H), 8.67 (br s, 1H), 8.64 (d, 1H), 8.11 (d, 1H), 7.72 (dd, 1H), 7.63 (d, 2H), 7.54 (d, 2H), 7.24 (d, 2H), 6.95 (d, 2H), 6.80 (t, 1H), 4.36 (d, 2H), 1.26 (s, 9H) |
| 282 | 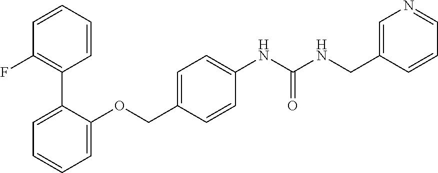 [SMILES: O=C(NCc1cccnc1)Nc1ccc(COc2ccccc2-c2ccccc2F)cc1] | 8.63 (s, 1H), 8.52 (br s, 1H), 8.45 (d, 1H), 7.70 (dt, 1H), 7.39-7.33 (m, 6H), 7.26-7.22 (m, 3H), 7.17 (d, 3H), 7.03 (td, 1H), 6.71 (t, 1H), 5.00 (s, 2H), 4.30 (d, 2H) |
| 283 | 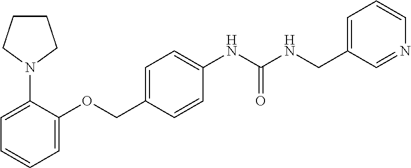 [SMILES: O=C(NCc1cccnc1)Nc1ccc(COc2ccccc2N2CCCC2)cc1] | 8.96 (s, 1H), 8.71 (d, 1H), 8.69-8.65 (m, 1H), 8.16 (d, 1H), 7.78-7.73 (m, 1H), 7.44 (d, 2H), 7.36 (d, 2H), 7.20- 6.90 (m, 5H), 5.05 (s, 2H), 4.42 (d, 2H), 3.51-3.31 (m4H), 1.97-1.81 (m, 4H) |
| 284 | 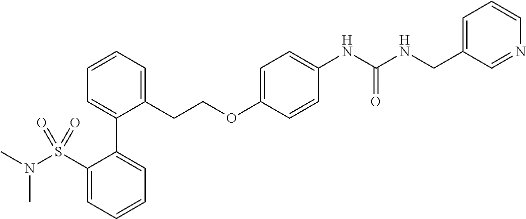 [SMILES: CN(C)S(=O)(=O)c1ccccc1-c1ccccc1CCOc1ccc(NC(=O)NCc2cccnc2)cc1] | 8.51 (br s, 1H), 8.44 (dd, 1H), 8.42 (br s, 1H), 8.38 (s, 1H), 7.97 (dd, 1H), 7.71-7.68 (m, 1H), 7.65-7.60 (m, 1H), 7.44 (t, 1H), 7.39-7.26 (m, 4H), 7.25-7.18 (m, 2H), 7.14 (dd, 1H), 6.82 (d, 1H), 6.64 (d, 1H), 6.62-6.56 (m, 1H), 4.29 (d, 2H), 4.12 (t, |
| 285 | 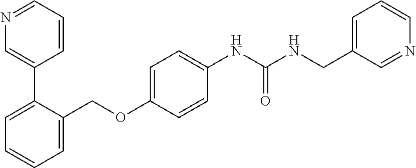 [SMILES: O=C(NCc1cccnc1)Nc1ccc(OCc2ccccc2-c2cccnc2)cc1] | 8.76-8.61 (m, 5H), 8.20 (d, 1H), 8.06 (d, 1H), 7.80 (dd, 1H), 7.66-7.59 (m, 2H), 7.52- 7.48 (m, 2H), 7.42-7.39 (m, 1H), 7.25 (d, 2H), 6.79 (t, 1H), 6.76 (d, 2H), 4.90 (s, 2H), 4.40 (d, 2H). |
| 286 | 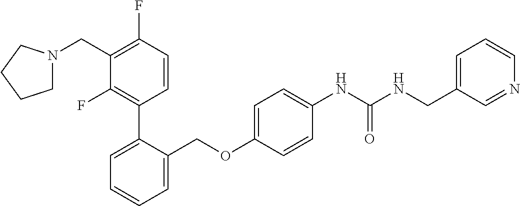 [SMILES: O=C(NCc1cccnc1)Nc1ccc(OCc2ccccc2-c2ccc(F)c(CN3CCCC3)c2F)cc1] | 10.08 (bs, 1H), 8.70 (bs, 1H), 8.63 (s, 1H), 8.05 (d, 2H), 7.70 (bs, 1H), 7.65-7.57 (m, 2H), 7.53-7.44 (m, 2H), 7.36- 7.29 (m, 2H), 7.23 (d, 2H), 6.83 (t, 1H), 6.70 (d, 2H), 4.85 (s, 2H), 4.49 (s, 2H), 4.37 (d, 2H), 3.45 (m, 2H), 3.45 (m, 2H), 1.99 (m, 2H) |
| 287 | 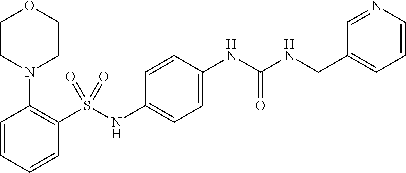 [SMILES: O=C(NCc1cccnc1)Nc1ccc(NS(=O)(=O)c2ccccc2N2CCOCC2)cc1] | 8.57 (s, 1H), 8.48 (s, 1H), 8.43 (d, 1H), 7.79 (d, 1H), 7.66 (d, 1H), 7.56 (t, 1H), 7.45 (d, 1H), 7.33 (dd, 1H), 7.24 (t, 1H), 7.18 (d, 2H), 6.87 (d, 2H), 6.69 (t, 1H), 4.26 (d, 2H), 2.94 (br s, 4H), 2.68 (br s, 4H), 2.32 (s, 3H). |
| 288 | 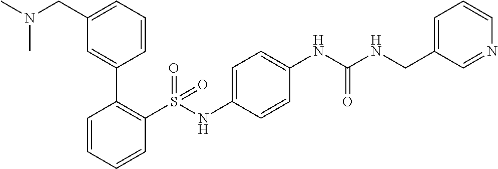 [SMILES: CN(C)Cc1cccc(-c2ccccc2S(=O)(=O)Nc2ccc(NC(=O)NCc3cccnc3)cc2)c1] | 9.87 (s, 1H), 9.73 (bs, 1H), 8.75 (s, 1H), 8.66-8.58 (m, 2H), 8.01 (d, 1H), 7.98-7.94 (m, 1H), 7.68-7.56 (m, 3H), 7.52-7.47 (m, 2H), 7.38-7.27 (m, 3H), 7.23 (d, 2H), 6.87 (t, 1H), 6.82 (d, 2H), 4.35 (d, 2H), 4.30 (d, 2H), 2.75 (s, 6H) |
| 289 | 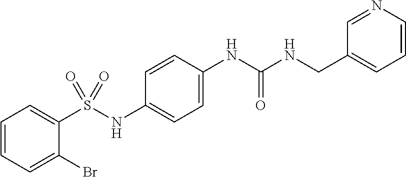 [SMILES: O=C(NCc1cccnc1)Nc1ccc(NS(=O)(=O)c2ccccc2Br)cc1] | 10.27 (s, 1H), 8.68 (br s, 1H), 8.37-8.60 (m, 2H), 8.05-8.02 (m, 1H), 7.98-7.95 (m, 1H), 7.65 (dd, 1H), 7.52-7.49 (m, 2H), 7.22 (d, 2H), 6.95 (d, 2H), 6.78 (t, 1H), 4.34 (d, 2H) |
| 290 | 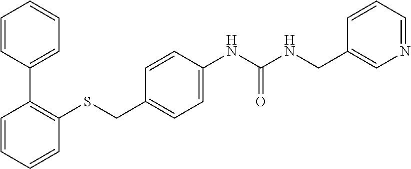 [SMILES: O=C(NCc1cccnc1)Nc1ccc(CSc2ccccc2-c2ccccc2)cc1] | 8.71 (s, 1H), 8.63 (d, 1H), 8.59 (dd, 1H), 7.99 (bs, 1H), 7.61 (bs, 1H), 7.48-7.19 (m, 13H), 7.14 (d, 2H), 6.75 (t, 1H), 4.36 (d, 2H), 4.04 (s, 2H) |
| 291 | 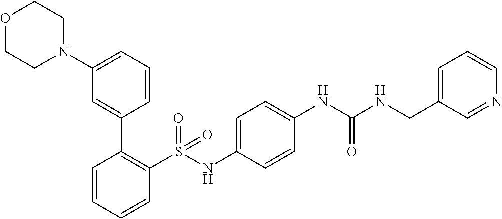 [SMILES: O=C(NCc1cccnc1)Nc1ccc(NS(=O)(=O)c2ccccc2-c2cccc(N3CCOCC3)c2)cc1] | 9.71 (s, 1H), 8.81-8.70 (m, 3H), 8.30 (d, 1H), 7.98-7.94 (m, 1H), 7.91-7.86 (m, 1H), 7.64-7.50 (m, 2H), 7.31-7.18 (m, 4H), 7.00-6.95 (m, 1H), 6.87 (t, 1H), 6.81 (d, 2H), 6.77-6.74 (m, 1H), 6.70 (d, 1H), 4.41 (d, 2H), 3.76-3.70 (m, 4H), 3.13-3.06 (m, 4H) |
| 292 | 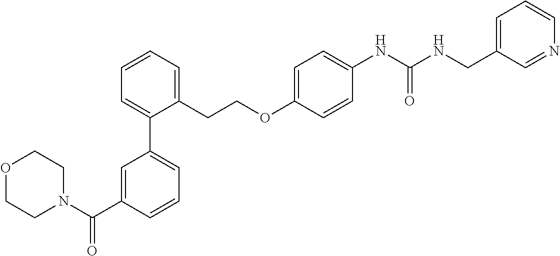 [SMILES: O=C(NCc1cccnc1)Nc1ccc(OCCc2ccccc2-c2cccc(C(=O)N3CCOCC3)c2)cc1] | 8.69 (d, 1H), 8.65 (dd, 1H), 8.55 (s, 1H), 8.15 (br d, 1H), 7.75 (dd, 1H), 7.56-7.30 (m, 7H), 7.24-7.21 (m, 3H), 7.18- 7.15 (m, 1H), 4.38 (d, 2H), 3.96 (t, 2H), 3.63-3.28 (m, 8H), 2.96 (t, 2H) |
| 293 | 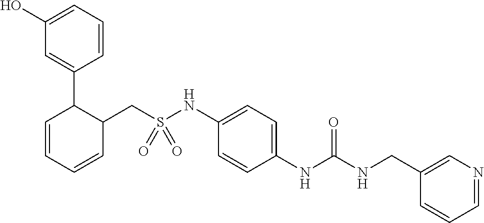 [SMILES: O=C(NCc1cccnc1)Nc1ccc(NS(=O)(=O)CC2C=CC=CC2c2cccc(O)c2)cc1] | 9.68 (s, 1H), 9.54 (s, 1H), 8.70 (s, 1H), 8.66 (s, 1H), 8.61 (d, 1H), 8.02 (d, 1H), 7.64 (dd, 1H), 7.49-7.45 (m, 1H), 7.39 (m, 2H), 3.2 4 (d, 2H), 7.25-7.22 (m, 1H), 7.15 (dd, 1H), 6.90 (d, 2H), 8.81- 6.68 (m, 4H), 4.38 (d, 2H), 4.34 (s, 2H) |
| 294 | 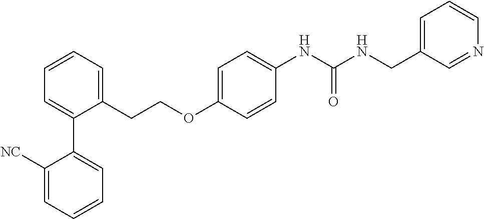 [SMILES: N#Cc1ccccc1-c1ccccc1CCOc1ccc(NC(=O)NCc2cccnc2)cc1] | 8.50 (d, 1H), 8.44 (dd, 1H), 8.39 (s, 1H), 7.97 (dd, 1H), 7.79 (td, 1H), 7.68 (dt, 1H), 7.61 (td, 1H), 7.57-7.53 (m, 2H), 7.45 (td, 1H), 7.39-7.33 (m, 2H), 7.25 (dd, 1H), 7.20 (d, 2H), 6.62 (d, 2H), 6.59 (t, 1H), 4.28 (d, 2H), 3.94 (m, 2H), 2.93-2.86 (m |
| 295 | 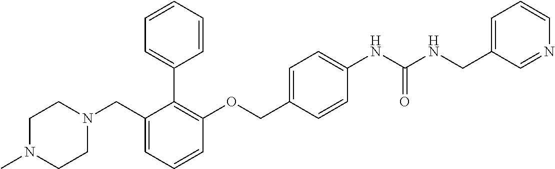 [SMILES: CN1CCN(Cc2cccc(OCc3ccc(NC(=O)NCc4cccnc4)cc3)c2-c2ccccc2)CC1] | 8.88 (s, 1H), 8.72 (s, 1H), 8.15 (d, 1H), 7.77 (s, 1H), 7.45- 7.38 (m, 2H), 7.38-7.28 (m, 4H), 7.26-7.20 (m, 2H), 7.16- 6.96 (m, 6H), 4.93 (s, 2H), 4.70-4.09 (m, 6H), 3.03-2.87 (m, 2H), 2.86-2.72 (m, 5H), 2.24-2.03 (m, 2H) |
| 296 | 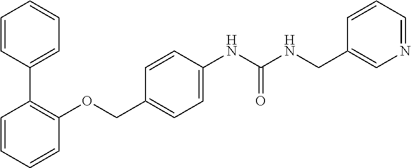 [SMILES: O=C(NCc1cccnc1)Nc1ccc(COc2ccccc2-c2ccccc2)cc1] | 8.65 (s, 1H), 8.52 (d, 1H), 8.45 (dd, 1H), 7.71 (dt, 1H), 7.51 (d, 2H), 7.42-7.27 (m, 8H), 7.23 (d, 2H), 7.18 (d, 1H), 7.03 (td, 1H), 6.71 (t, 1H), 5.02 (s, 2H), 4.31 (d, 2H) |
| 297 | 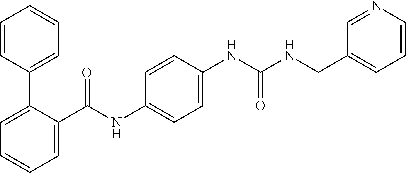 [SMILES: O=C(NCc1cccnc1)Nc1ccc(NC(=O)c2ccccc2-c2ccccc2)cc1] | 10.08 (s, 1H), 8.77-8.22 (m, 2H), 7.89-6.88 (m, 15H), 6.74- 6.48 (m, 1H), 4.30 (d, 2H), 3.92 (s, 1H) (DMSO) |
| 298 | 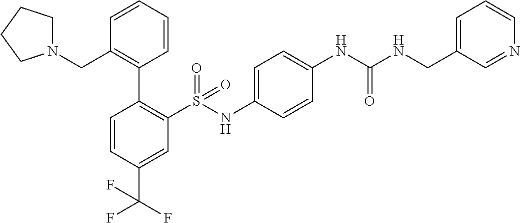 [SMILES: O=C(NCc1cccnc1)Nc1ccc(NS(=O)(=O)c2cc(C(F)(F)F)ccc2-c2ccccc2CN2CCCC2)cc1] | 10.10 (s, 1H), 9.61 (s, 1H), 8.78 (s, 1H), 8.64-8.59 (m, 1H), 8.59-8.55 (m, 1H), 8.33- 8.30 (m, 1H), 8.08-8.04 (m, 1H), 7.97-7.92 (m, 1H), 7.67 (d, 1H), 7.60-7.51 (m, 2H), 7.42-7.35 (m, 1H), 7.28 (d, 2H), 6.90-6.81 (m, 3H), 4.35 (d, 2H), 4.22-4.15 (m, 1H) |
| 299 | 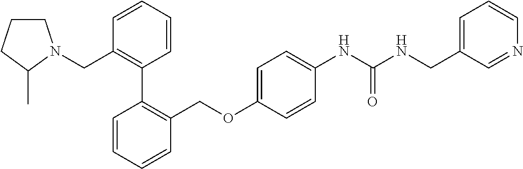 [SMILES: CC1CCCN1Cc1ccccc1-c1ccccc1COc1ccc(NC(=O)NCc2cccnc2)cc1] | 8.63 (t, 3 H), 8.03 (d, 1 H), 7.62-7.71 (m, 3 H), 7.47- 7.55 (m, 4 H), 7.30-7.39 (m, 2 H), 7.24 (dd, 2 H), 6.82 (t, 1 H), 6.66-6.72 (m, 2 H), 4.58-4.68 (m, 2 H), 4.41-4 4.49 (m, 0.4 H), 4.36 (d, 2 H), 4.16-4.24 (m, 0.6 H), 4.03- 4.11 (m, 0.6 |
| 300 | 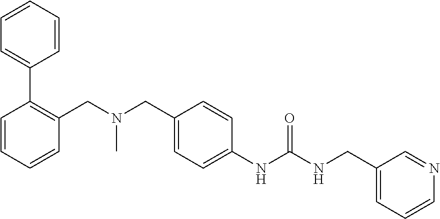 [SMILES: CN(Cc1ccc(NC(=O)NCc2cccnc2)cc1)Cc1ccccc1-c1ccccc1] | 8.57 (s, 1H), 8.52 (d, 1H), 8.45 (dd, 1H), 7.62 (d, 1H), 7.47-7.28 (m, 12H), 7.21 (dd, 1H), 7.06 (d, 2H), 6.66 (t, 1H), 4.31 (d, 2H), 3.42 (s, 2H), 3.27 (s, 2H), 1.92 (s, 3H) |
| 301 | 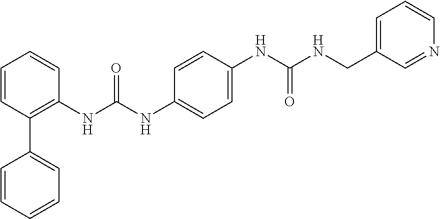 [SMILES: O=C(NCc1cccnc1)Nc1ccc(NC(=O)Nc2ccccc2-c2ccccc2)cc1] | 8.86 (s, 1H), 8.52 (d, 1H), 8.48 (s, 1H), 8.45 (dd, 1H), 7.94 (d, 1H), 7.71 (m, 1H), 7.57 (s, 1H), 7.51 (m, 2H), 7.45-7.22 (m, 8H), 7.20 (dd, 1H), 7.11 (dd, 1H), 6.63 (d, 1H), 4.30 (d, 2H) |
| 302 | 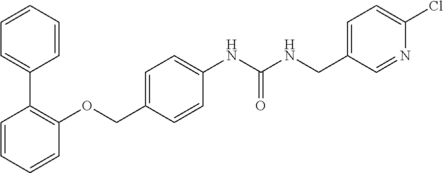 [SMILES: O=C(NCc1ccc(Cl)nc1)Nc1ccc(COc2ccccc2-c2ccccc2)cc1] | 8.71 (s, 1H), 8.35 (d, 1H), 7.78 (dd, 1H), 7.54-7.47 (m, 3H), 7.42-7.35 (m, 4H), 7.35- 7.26 (m, 3H), 7.23 (d, 2H), 7.18 (d, 1H), 7.03 (dd, 1H), 6.75 (dd, 1H), 5.02 (s, 1H), 4.30 (d, 2H) |
| 303 | 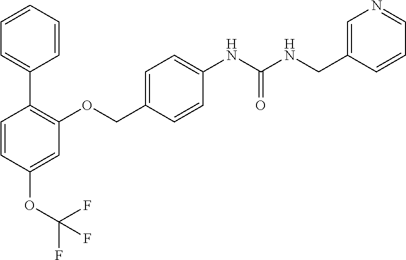 [SMILES: O=C(NCc1cccnc1)Nc1ccc(COc2cc(OC(F)(F)F)ccc2-c2ccccc2)cc1] | 8.67 (s, 1H), 8.52 (d, 1H), 8.45 (dd, 1H), 7.70 (dt, 1H), 7.52 (d, 2H), 7.45-7.21 (m, 7H), 7.02 (d, 1H), 6.73 (t, 1H), 5.07 (s, 2H), 4.31 (d, 2H) |
| 304 | 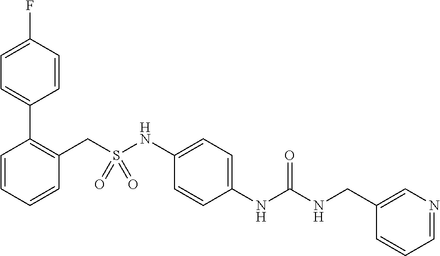 [SMILES: O=C(NCc1cccnc1)Nc1ccc(NS(=O)(=O)Cc2ccccc2-c2ccc(F)cc2)cc1] | 9.65 (s, 1H), 8.73 (s, 1H), 8.69 (s, 1H), 8.64 (d, 1H), 8.10 (d, 1H), 7.70 (dd, 1H), 7.52-7.47 (m, 1H), 7.45-7.40 (m, 2H), 7.34-7.23 (m, 5H), 7.16 (dd, 2H), 6.87 (d, 2H), 6.79 (dd, 1H), 4.40 (d, 2H), 4.30 (s, 2H) |
| 305 | 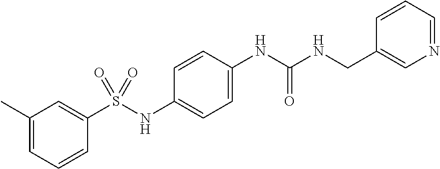 [SMILES: Cc1cccc(S(=O)(=O)Nc2ccc(NC(=O)NCc3cccnc3)cc2)c1] | 9.90 (s, 1H), 8.58 (s, 1H), 8.54 (d, 1H), 8.49 (d, 1H), 7.53 (s, 1H), 7.49-7.42 (m, 2H), 7.40 (d, 2H), 7.23 (d, 2H), 6.92 (d, 2H), 6.68 (t, 1H), 4.30 (d, 2H), 2.33 (s, 3H) |
| 306 | 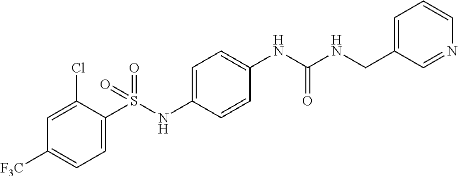 [SMILES: O=C(NCc1cccnc1)Nc1ccc(NS(=O)(=O)c2ccc(C(F)(F)F)cc2Cl)cc1] | 10.52 (s, 1H), 8.59 (s, 1H), 8.49 (s, 1H), 8.44 (d, 1H), 8.14-8.10 (m, 2H), 7.87 (d, 1H), 7.68-7.64 (m, 1H), 7.36- 7.31 (m, 1H), 7.26 (d, 2H), 6.97 (d, 2H), 6.66 (t, 1H), 4.27 (d, 2H) |
| 307 | 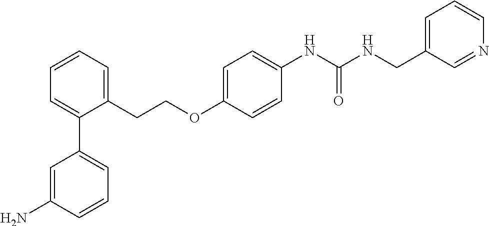 [SMILES: Nc1cccc(-c2ccccc2CCOc2ccc(NC(=O)NCc3cccnc3)cc2)c1] | 8.53 (d, 1H), 8.47 (dd, 1H), 8.39 (s, 1H), 7.72 (dt, 1H), 7.42-7.37 (m, 2H), 7.31-7.25 (m, 2H), 7.22 (d, 2H), 7.14 (dd, 1H), 7.07 (t, 1H), 6.65 (d, 2H), 6.60-6.56 (m, 2H), 6.51 (t, 1H), 6.44 (dt, 1H), 5.16 (s, 2H), 4.30 (d, 2H), 3.95 (t, 2H)2.97 (t, 2H) |
| 308 | 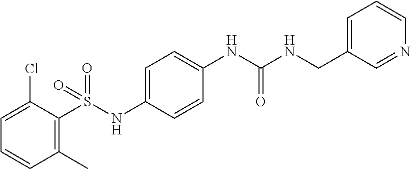 [SMILES: Cc1cccc(Cl)c1S(=O)(=O)Nc1ccc(NC(=O)NCc2cccnc2)cc1] | 10.21 (s, 1H), 8.85-8.31 (m, 3H), 7.89-7.73 (m, 1H), 7.68- 6.53 (m, 9H), 4.39 (d, 2H), 2.65 (s, 3H) |
| 309 | 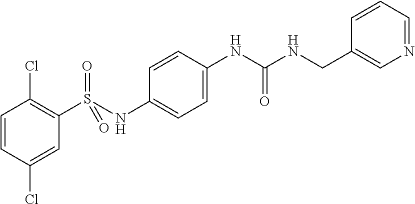 [SMILES: O=C(NCc1cccnc1)Nc1ccc(NS(=O)(=O)c2cc(Cl)ccc2Cl)cc1] | 10.43 (s, 1H), 8.59 (s, 1H), 8.52-8.47 (m, 1H), 8.47-8.41 (m, 1H), 7.89-7.84 (m, 1H), 7.74-7.64 (m, 3H), 7.37-7.30 (m, 1H), 7.27 (d, 2H), 6.97 (d, 2H), 6.67 (t, 1H), 4.28 (d, 2H) |
| 310 | 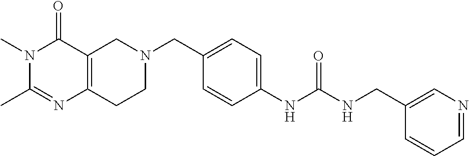 [SMILES: Cc1nc2c(c(=O)n1C)CN(Cc1ccc(NC(=O)NCc3cccnc3)cc1)CC2] | 10.18 (bs, 1H), 9.19 (s, 1H), 8.75 (s, 1H), 8.71 (d, 1H), 8.22 (d, 1H), 7.86-7.77 (m, 1H), 7.52 (d, 2H), 7.39 (d, 2H), 7.17 (t, 1H), 4.44 (d, 2H), 4.40 (s, 3H), 4.06-3.83 (m, 2H), 3.62 (bs, 1H), 3.44 (bs, 1H), 2.91-2.77 (m, 2H) |
| 311 | 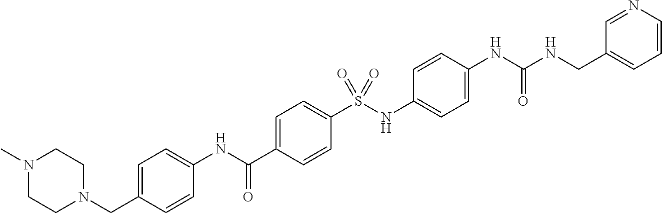 [SMILES: CN1CCN(Cc2ccc(NC(=O)c3ccc(S(=O)(=O)Nc4ccc(NC(=O)NCc5cccnc5)cc4)cc3)cc2)CC1] | 10.51 (s, 1H), 10.14 (s, 1H), 8.84 (s, 1H), 8.07-8.01 (m, 2H), 7.82 (d, 2H), 7.75 (d, 2H), 7.72-7.67 (m, 1H), 7.36 (d, 2H), 7.27 (t, 3H), 7.14 (s, 2H), 7.01 (s, 2H), 6.97-6.89 (m, 3H), 4.36 (d, 2H), 2.79 (s, 3H) |
| 312 | 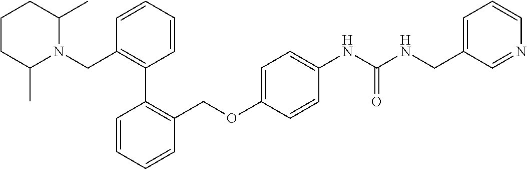 [SMILES: CC1CCCC(C)N1Cc1ccccc1-c1ccccc1COc1ccc(NC(=O)NCc2cccnc2)cc1] | 9.06 (br s, 1 H), 8.69-8.49 (m, 3 H), 8.46-8.27 (m, 1 H), 8.02-7.90 (m, 1 H), 7.76- 7.40 (m, 7 H), 7.40-7.29 (m, 2 H), 7.29-7.14 (m, 2 H), 6.97-6.65 (m, 3 H), 5.74- 4.63 (m, 2 H), 4.39-4.24 (m, 4 H), 4.07-3.99 (m, 2 H), 3.50-3.41 (m, 2 |
| 313 | 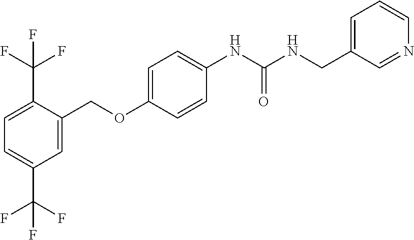 [SMILES: O=C(NCc1cccnc1)Nc1ccc(OCc2cc(C(F)(F)F)ccc2C(F)(F)F)cc1] | 8.69 (s, 1H), 8.65 (d, 1H), 8.63 (s, 1H), 8.15-8.09 (m, 2H), 8.06 (d, 1H), 7.98 (d, 1H), 7.72 (dd, 1H), 7.33 (d, 2H), 6.93 (d, 2H), 6.76 (t, 1H), 5.26 (s, 2H), 4.39 (d, 2H) |
| 314 | 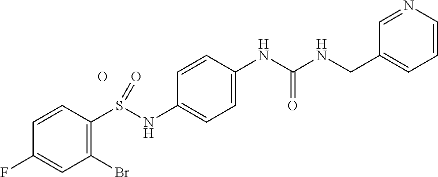 | 10.31 (s, 1H), 8.57 (br s, 1H), 8.49-8.48 (m, 1H), 8.44 (dd, 1H), 8.01 (dd, 1H), 7.83 (dd, 1H), 7.68-7.65 (m, 1H), 7.42-7.37 (m, 1H), 7.34 (ddd, 1H), 7.24 (d, 2H), 6.95 (d, 2H), 6.66 (t, 1H), 4.27 (d, 2H) |
| 315 | 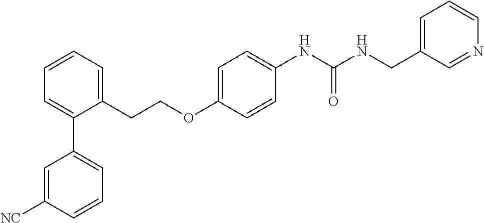 [SMILES: N#Cc1cccc(-c2ccccc2CCOc2ccc(NC(=O)NCc3cccnc3)cc2)c1] | 8.51 (br s, 1H), 8.45 (d, 1H), 8.40 (s, 1H), 7.89-7.87 (m, 1H), 7.86 (t, 1H), 7.74-7.64 (m, 3H), 7.50 (dd, 1H), 7.42- 7.31 (m, 3H), 7.25-7.21 (m, 3H), 6.66 (d, 2H), 6.58 (t, 1H), 4.29 (d, 2H), 4.00 (t, 2H), 2.93 (t, 2H) |
| 316 | 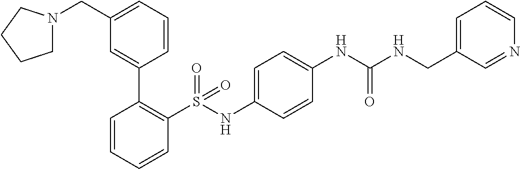 [SMILES: O=C(NCc1cccnc1)Nc1ccc(NS(=O)(=O)c2ccccc2-c2cccc(CN3CCCC3)c2)cc1] | 9.86 (s, 2H), 8.75 (s, 1H), 8.64-8.58 (m, 2H), 8.01-7.95 (m, 2H), 7.68-7.56 (m, 3H), 7.54-7.45 (m, 2H), 7.38-7.27 (m, 3H), 7.24 (d, 2H), 6.87 (t, 1H), 6.81 (d, 2H), 4.39-4.33 (m, 4H), 3.45-3.32 (m, 2H), 3.15-3.02 (m, 2H), 2.09-1.93 (m, 2H), 1.92-1.77 (m, |
| 317 | 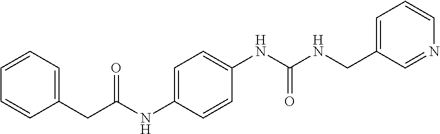 [SMILES: O=C(Cc1ccccc1)Nc1ccc(NC(=O)NCc2cccnc2)cc1] | d 8.59 (m, 1H), 8.56 (bs, 2H), 7.71 (bs, 2H), 7.50 (bs, 1H), 7.38 (m, 3H), 7.09 (d, 1H), 4.00 (t, 2H), 3.38 (q, 1H), 3.32 (m, 3H), 2.83 (dt, 2H), 1.76 (m, 4H), 1.59 (p, 2H), 1.42 (m, 4H) |
| 318 | 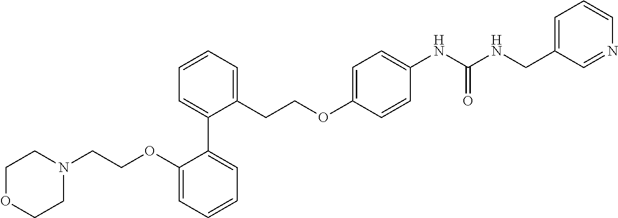 [SMILES: O=C(NCc1cccnc1)Nc1ccc(OCCc2ccccc2-c2ccccc2OCCN2CCOCC2)cc1] | 8.51 (d, 1H), 8.45 (dd, 1H), 8.39 (s, 1H), 7.70-7.67 (dt, 1H), 7.41-7.23 (m, 5H), 7.19 (d, 2H), 7.16 (dd, 1H), 7.12- 7.08 (m, 2H), 7.04 (td, 1H), 6.60-6.54 (m, 3H), 4.29 (d, 2H), 4.02 (td, 2H), 3.91-3.86 (m, 2H), 3.41-3.37 (m, 4H), 2.84-2.79 (m, 2H), 2.4 |
| 319 | 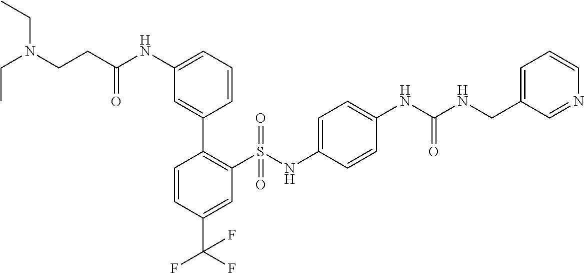 [SMILES: CCN(CC)CCC(=O)Nc1cccc(-c2ccc(C(F)(F)F)cc2S(=O)(=O)Nc2ccc(NC(=O)NCc3cccnc3)cc2)c1] | 10.37 (s, 1H), 10.09 (s, 1H), 9.31 (bs, 1H), 8.78 (s, 1H), 8.62 (d, 1H), 8.60-8.56 (m, 1H), 8.25 (s, 1H), 8.03-7.99 (m, 1H), 7.96 (d, 1H), 7.67- 7.56 (m, 3H), 7.53 (d, 1H), 7.36 (t, 1H0; 7.26 (d, 2H), 6.94-9.82 (m, 4H), 4.35 (d, 2H), 4.16-3.50 (m, 2H), 3. |
| 320 | 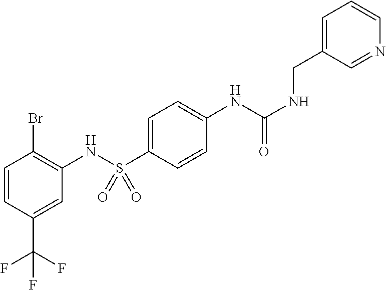 [SMILES: O=C(NCc1cccnc1)Nc1ccc(S(=O)(=O)Nc2cc(C(F)(F)F)ccc2Br)cc1] | (400 MHz, MeOH-d4) 8.58 (bs, 3H), 8.00 (d, 1H), 7.91 (d, 2H), 7.80-7.72 (m, 4H), 7.67 (dd, 2H), 7.60 (d, 2H), 7.53 (d, 2H), 7.34 (d, 1H), 4.64 (d, 4H) |
| 321 | 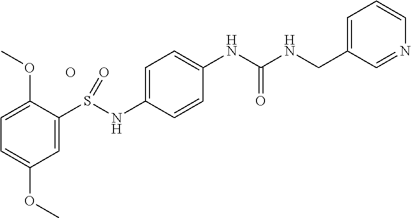 | 9.65 (s, 1H), 8.53-8.41 (3H), 7.66 (d, 1H), 7.37-7.30 (m, 1H), 7.20 (d, 2H), 7.17-7.08 (m, 3H), 6.94 (d, 2H), 6.63 (t, 1H), 4.27 (d, 2H), 3.85 (s, 3H), 3.69 (s, 3H) |
| 322 | 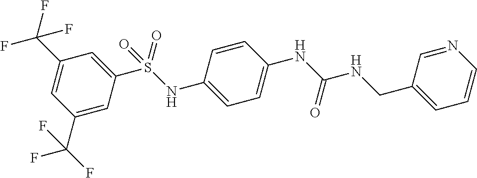 [SMILES: O=C(NCc1cccnc1)Nc1ccc(NS(=O)(=O)c2cc(C(F)(F)F)cc(C(F)(F)F)c2)cc1] | 10.25 (s, 1H), 8.80 (s, 1H), 8.66 (s, 1H), 8.63 (d, 1H), 8.50 (s, 1H), 8.19 (s, 2H), 8.08 (d, 1H), 7.72-7.65 (m, 1H), 7.30 (d, 2H), 6.91 (d, 2H), 6.82 (t, 1H), 4.37 (d, 2H) |
| 323 | 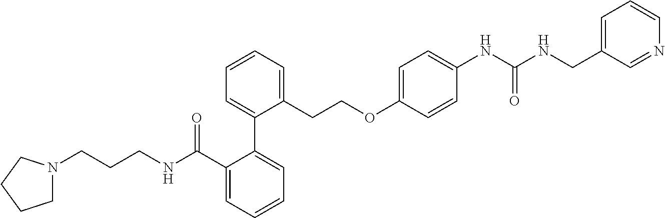 [SMILES: O=C(NCc1cccnc1)Nc1ccc(OCCc2ccccc2-c2ccccc2C(=O)NCCCN2CCCC2)cc1] | 8.65 (d, 1H), 8.62 (dd, 1H), 8.57 (s, 1H), 8.28 (t, 1H), 8.04 (dt, 1H), 7.67 (dd, 1H), 7.53-7.47 (m, 3H), 7.42 (dd, 1H), 7.35-7.23 (m, 3H), 7.20 (d, 2H), 7.15 (dd, 1H), 6.77 (t, 1H), 6.61 (d, 2H), 4.36 (d, 2H), 4.02-3.88 (m, 2H), 3.44- 3.35 (m, 2H), 3.18 |
| 324 | 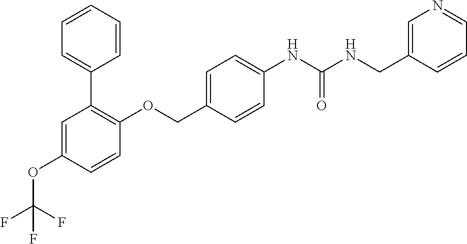 [SMILES: O=C(NCc1cccnc1)Nc1ccc(COc2ccc(OC(F)(F)F)cc2-c2ccccc2)cc1] | 8.84 (s, 1H), 8.73-8.65 (m, 2H), 8.13 (dt, 1H), 7.75-7.71 (m, 1H), 7.54 (d, 2H), 7.44- 7.32 (m, 5H), 7.29-7.22 (m, 3H), 6.87 (t, 1H), 5.05 (s, 2H), 4.40 (d, 2H) |
| 325 | 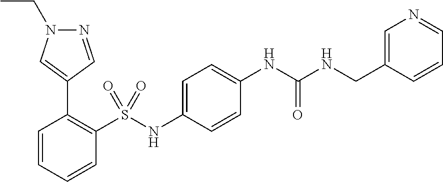 [SMILES: CCn1cc(-c2ccccc2S(=O)(=O)Nc2ccc(NC(=O)NCc3cccnc3)cc2)cn1] | 9.72 (s, 1H), 8.68-8.64 (m, 3H), 8.11 (d, 1H), 7.96-7.95 (m, 2H), 7.93-7.89 (m, 1H), 7.72 (dd, 1H), 7.64-7.54 (m, 3H), 7.46-7.38 (m, 3H), 7.18 (d, 2H), 6.97 (d, 1H), 6.86 (dd, 1H), 6.80-6.75 (m, 3H), 4.36 (d, 2H), 4.21-4.15 (m, 3H), 1.44-1.40 (m, 4H) |
| 326 | 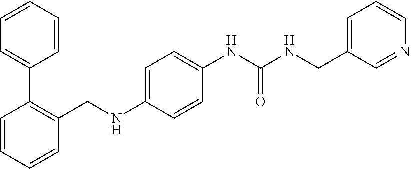 [SMILES: O=C(NCc1cccnc1)Nc1ccc(NCc2ccccc2-c2ccccc2)cc1] | 8.70 (s, 1H), 9.69 (d, 1H), 8.35 (s, 1H), 8.21 (d, 1H), 7.80 (dd, 1H), 7.52 (dd, 1H), 7.48-7.31 (m, 8H), 7.24 (dd, 1H), 7.05 (d, 2H), 6.67 (t, 1H), 6.44 (d, 2H), 4.37 (d, 2H), 4.11 (s, 2H) |
| 327 | 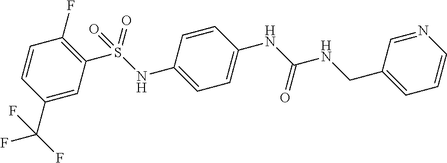 [SMILES: O=C(NCc1cccnc1)Nc1ccc(NS(=O)(=O)c2cc(C(F)(F)F)ccc2F)cc1] | 10.51 (s, 1H), 8.85 (s, 1H), 8.71-8.62 (m, 2H), 8.17-8.09 (m, 2H), 7.99-7.93 (m, 1H), 7.78-7.66 (m, 2H), 7.29 (d, 2H), 6.97 (d, 2H), 6.87 (t, 1H), 4.37 (d, 2H) |
| 328 | 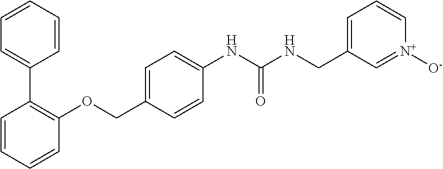 [SMILES: O=C(NCc1ccc[n+]([O-])c1)Nc1ccc(COc2ccccc2-c2ccccc2)cc1] | 8.76 (s, 1H), 8.14 (s, 1H), 8.11 (d, 1H), 7.51 (dd, 2H), 7.42-7.26 (m, 9H), 7.24 (d, 2H), 7.18 (d, 1H), 7.03 (t, 1H), 6.75 (t, 1H), 5.02 (s, 2H), 4.25 (d, 2H) |
| 329 | 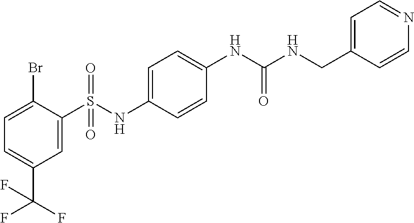 [SMILES: O=C(NCc1ccncc1)Nc1ccc(NS(=O)(=O)c2cc(C(F)(F)F)ccc2Br)cc1] | 9.94 (s, 1H), 8.39 (d, 2H), 8.25-8.21 (m, 1H), 8.09 (d, 1H), 7.95-7.89 (m, 1H), 7.57 (d, 2H), 7.23-7.15 (m, 3H), 7.07 (d, 2H), 4.21 (d, 2H) |
| 330 | 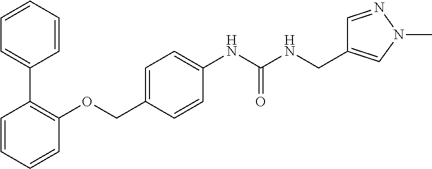 [SMILES: Cn1cc(CNC(=O)Nc2ccc(COc3ccccc3-c3ccccc3)cc2)cn1] | 8.48 (s, 1H), 7.58 (s, 1H), 7.52 (d, 2H), 7.44-7.27 (m, 8H), 7.26-7.16 (m, 3H), 7.06- 7.01 (m, 1H), 6.37 (t, 1H), 5.02 (s, 2H), 4.09 (d, 2H), 3.78 (s, 3H) |
| 331 | 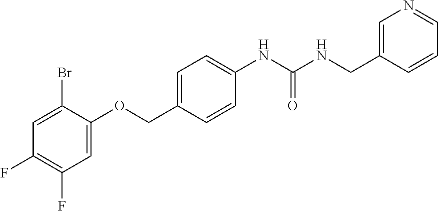 [SMILES: O=C(NCc1cccnc1)Nc1ccc(COc2cc(F)c(F)cc2Br)cc1] | 8.72 (s, 1H), 8.52 (d, 1H), 8.45 (dd, 1H), 7.84 (dd, 1H), 7.72 (dt, 1H), 7.48-7.42 (m, 3H), 7.41-7.33 (m, 3H), 6.74 (t, 1H), 5.08 (s, 2H), 4.32 (d, 2H) |

TABLE 1B

| | | HRMS | |
|---|---|---|---|
| Example | | Found | Synthetic |
| Number | IUPAC Name | [M + H]⁺ | Procedures |
| 1 | 3′-[(methylsulfonyl)amino]-N-(4-{[(pyridin-3- | 552.1361 | 1, 2, 3, 4 |
| | ylmethyl)carbamoyl]amino}phenyl)biphenyl-2- | | |
| | sulfonamide | | |
| 2 | 1-(4-{[2-(4-Chloropyridin-3-yl)benzyl]oxy}phenyl)- | 445.1421 | 37, 32, 18, 4 |
| | 3-(pyridin-3-ylmethyl)urea | | |
| 3 | 2-Methoxy-5-methyl-N-(4-{[(pyridin-3- | 427.1432 | 1, 2, 3 |
| | ylmethyl)carbamoyl]amino}phenyl)benzenesulfonamide | | |
| 4 | 1-(4-{[(2-Phenylpyridin-3-yl)oxy]methyl}phenyl)-3- | 411.1786 | 7, 8, 4 |
| | (pyridin-3-ylmethyl)urea | | |
| 5 | 2,6-dichloro-N-(4-{[(pyridin-3- | 451.0404 | 1, 2, 3 |
| | ylmethyl)carbamoyl]amino}phenyl)benzenesulfonamide | | |
| 6 | N-(4-{[(Pyridin-3- | 467.1001 | 1, 2, 3 |
| | ylmethyl)carbamoyl]amino}phenyl)-3- | | |
| | (trifluoromethoxy)benzenesulfonamide | | |
| 7 | 4-bromo-N-(4-{[(pyridin-3- | 531.0194 | 1, 2, 3 |
| | ylmethyl)carbamoyl]amino}phenyl)-3- | | |
| | (trifluoromethyl)benzenesulfonamide | | |
| 8 | 1-{4-[(2-{1-[2-(Dimethylamino)ethyl]-1H-pyrazol-4- | 471.2487 | 37, 32, 18, 90, |
| | yl}benzyl)oxy]phenyl}-3-(pyridin-3-ylmethyl)urea | | 91 |
| 9 | N,N-Dimethyl-2′-[2-(4-{[(pyridin-3- | 531.2077 | 10, 45, 46, 4 |
| | ylmethyl)carbamoyl]amino}phenoxy)ethyl]biphenyl- | | |
| | 3-sulfonamide | | |
| 10 | N-(Biphenyl-2-yl)-2-[(4-{[(pyridin-3- | 516.2000 | 1, 2, 3, 33, 9 |
| | ylmethyl)carbamoyl]amino}phenyl)sulfamoyl]acetamide | | |
| 11 | 1-(4-{[(5-Fluorobiphenyl-2-yl)oxy]methyl}phenyl)-3- | 428.1729 | 7, 8, 4 |
| | (pyridin-3-ylmethyl)urea | | |
| 12 | 1-(4-{[2-(Piperidin-1-yl)phenoxy]methyl}phenyl)-3- | 417.2304 | 7, 8 |
| | (pyridin-3-ylmethyl)urea | | |
| 13 | | 467.0992 | 1, 2, 3 |
| 14 | 1-(Pyridin-3-ylmethyl)-3-(4-{[2-(pyridin-4- | 411.1854 | 7, 8, 4 |
| | yl)phenoxy]methyl}phenyl)urea | | |
| 15 | 1-{4-[({3′-[(Diethylamino)methyl]biphenyl-2- | 495.2809 | 35, 4, 18, 32, |
| | yl}oxy)methyl]phenyl}-3-(pyridin-3-ylmethyl)urea | | 37 |
| 16 | N-{4-[(1H-benzimidazol-6- | 484.1448 | 17, 18 |
| | ylcarbamoyl)amino]phenyl}biphenyl-2-sulfonamide | | |
| 17 | 1-(Pyridin-3-ylmethyl)-3-[4-({[3′-(pyrrolidin-1- | 493.2641 | 44, 20, 46, 4, |
| | ylmethyl)biphenyl-2-yl]oxy}methyl)phenyl]urea | | 48 |
| 18 | 1-{4-[(2-Chlorophenoxy)methyl]phenyl}-3-(pyridin- | 368.1169 | 7, 8 |
| | 3-ylmethyl)urea | | |
| 19 | 2-(1H-Imidazol-1-yl)-N-(4-{[(pyridin-3- | 449.1342 | 17, 18, 76 |
| | ylmethyl)carbamoyl]amino}phenyl)benzenesulfonamide | | |
| 20 | N-(4-{[(pyridin-3- | 527.1389 | 1, 2, 3, 4 |
| | ylmethyl)carbamoyl]amino}phenyl)-4- | | |
| | (trifluoromethyl)biphenyl-2-sulfonamide | | |
| 21 | 4-oxo-N-(4-{[(pyridin-3- | 451.1189 | 20, 21 |
| | ylmethyl)carbamoyl]amino}phenyl)-3,4- | | |
| | dihydroquinazoline-8-sulfonamide | | |
| 22 | N~2~,N~2~-Dimethyl-N-{2′-[2-(4-{[(pyridin-3- | 524.2649 | 10, 45, 46, 4, |
| | ylmethyl)carbamoyl]amino}phenoxy)ethyl]biphenyl- | | 40 |
| | 2-yl}glycinamide | | |
| 23 | N-{2′-[(4-{[(Pyridin-3- | 503.1693 | 44, 20, 46, 4 |
| | ylmethyl)carbamoyl]amino}benzyl)oxy]biphenyl-3- | | |
| | yl}methanesulfonamide | | |
| 24 | 1-[4-(2-{2′-[2-(Dimethylamino)ethoxy]biphenyl-2- | 511.2702 | 10, 4 |
| | yl}ethoxy)phenyl]-3-(pyridin-3-ylmethyl)urea | | |
| 25 | 1-(4-{[2-(2-{[2- | 511.2846 | 37, 32, 18, 4, |
| | (Dimethylamino)ethyl](methyl)amino}pyridin-4- | | 89 |
| | yl)benzyl]oxy}phenyl)-3-(pyridin-3-ylmethyl)urea | | |
| 26 | 2-(4-methylpiperazin-1-yl)-N-(4-{[(pyridin-3- | 480.2023 | 1, 2, 3, 22 |
| | ylmethyl)carbamoyl]amino}phenyl)benzenesulfonamide | | |
| 27 | 1-[6-(3-Aminophenyl)cyclohexa-2,4-dien-1-yl]-N-(4- | 488.1765 | 5, 18, 4 |
| | {[(pyridin-3- | | |
| | ylmethyl)carbamoyl]amino}phenyl)methanesulfonamide | | |
| 28 | N-(4-{[(Pyridin-3- | 465.1201 | 1, 2, 3 |
| | ylmethyl)carbamoyl]amino}phenyl)-1-[2- | | |
| | (trifluoromethyl)phenyl]methanesulfonamide | | |
| 29 | 1-{4-[1-(Biphenyl-2-yloxy)-2,2,2- | 478.1736 | 46, 71, 10 |
| | trifluoroethyl]phenyl}-3-(pyridin-3-ylmethyl)urea | | |
| 30 | 1-(4-{[2-(2-Methyl-1H-imidazol-1- | 414.1935 | 44, 32, 18, 76 |
| | yl)benzyl]oxy}phenyl)-3-(pyridin-3-ylmethyl)urea | | |
| 31 | 1-(4-{[(4′,5-Difluorobiphenyl-2- | 446.1698 | 7, 8, 4 |
| | yl)oxy]methyl}phenyl)-3-(pyridin-3-ylmethyl)urea | | |
| 32 | 1-(4-{[(3′-Fluorobiphenyl-2-yl)oxy]methyl}phenyl)- | 428.1784 | 7, 8, 4 |
| | 3-(pyridin-3-ylmethyl)urea | | |
| 33 | 3-(4-Methylpiperazin-1-yl)-N-{2′-[(4-{[(pyridin-3- | 696.2563 | 1, 2, 3, 4, 52, 9 |
| | ylmethyl)carbamoyl]amino}phenyl)sulfamoyl]-4′- | | |
| | (trifluoromethyl)biphenyl-3-yl}propanamide | | |
| 34 | 5-bromo-6-chloro-N-(4-{[(pyridin-3- | 495.9812 | 1, 2, 3 |
| | ylmethyl)carbamoyl]amino}phenyl)pyridine-3- | | |
| | sulfonamide | | |
| 35 | 1-{4-[(Biphenyl-2-ylmethyl)(propyl)amino]phenyl}- | 451.2441 | 1, 2, 35, 35 |
| | 3-(pyridin-3-ylmethyl)urea | | |
| 36 | 2-(piperidin-1-yl)-N-(4-{[(pyridin-3- | 466.1914 | 1, 2, 3, 22 |
| | ylmethyl)carbamoyl]amino}phenyl)benzenesulfonamide | | |
| 37 | 1-(4-{[2-(1H-Imidazol-1-yl)benzyl]oxy}phenyl)-3- | 400.1785 | 44, 32, 18, 76 |
| | (pyridin-3-ylmethyl)urea | | |
| 38 | N-(4-{[(pyridin-3- | 465.1056 | 1, 2, 3, 4 |
| | ylmethyl)carbamoyl]amino}phenyl)-2-(thiophen-3- | | |
| | yl)benzenesulfonamide | | |
| 39 | N-(5-{2-[(4-{[(Pyridin-3- | 468.2030 | 37, 32, 18, 4 |
| | ylmethyl)carbamoyl]amino}phenoxy)methyl]phenyl}pyridin- | | |
| | 2-yl)acetamide | | |
| 40 | 1-(4-{[(4′-Fluorobiphenyl-2-yl)oxy]methyl}phenyl)- | 428.1785 | 7, 8, 4 |
| | 3-(pyridin-3-ylmethyl)urea | | |
| 41 | N-{2′-[(4-{[(pyridin-3- | 516.1668 | 1, 2, 3, 4 |
| | ylmethyl)carbamoyl]amino}phenyl)sulfamoyl]biphenyl- | | |
| | 3-yl}acetamide | | |
| 42 | 1-(4-{[(Biphenyl-2-ylmethyl)(3-methylbut-2-en-1- | 491.2766 | 35, 72, 32, 18 |
| | yl)amino]methyl}phenyl)-3-(pyridin-3-ylmethyl)urea | | |
| 43 | 1-[4-({2-[2-(4-Methylpiperazin-1-yl)pyridin-4- | 509.3000 | 37, 32, 18, 4, |
| | yl]benzyl}oxy)phenyl]-3-(pyridin-3-ylmethyl)urea | | 89 |
| 44 | N-(4-{[(pyridin-4- | 459.1545 | 17, 18 |
| | ylcarbamoyl)amino]methyl}phenyl)biphenyl-2- | | |
| | sulfonamide | | |
| 45 | 2-(Biphenyl-2-yloxy)-N-{3-[(pyridin-4- | 405.2037 | 10, 15, 39, 18 |
| | ylcarbamoyl)amino]propyl}acetamide | | |
| 46 | 1-(4-{[(Biphenyl-2-ylmethyl)(prop-2-yn-1- | 461.2303 | 35, 72, 32, 18 |
| | yl)amino]methyl}phenyl)-3-(pyridin-3-ylmethyl)urea | | |
| 47 | 2,4-dichloro-5-methyl-N-(4-{[(pyridin-3- | 465.0599 | 1, 2, 3 |
| | ylmethyl)carbamoyl]amino}phenyl)benzenesulfonamide | | |
| 48 | 1-{4-[2-(2′-Hydroxybiphenyl-2-yl)ethoxy]phenyl}-3- | 440.1947 | 10, 45, 46, 4 |
| | (pyridin-3-ylmethyl)urea | | |
| 49 | 1-(2-Bromophenyl)-N-(4-{[(pyridin-3- | 477.0421 | 1, 2, 3 |
| | ylmethyl)carbamoyl]amino}phenyl)methanesulfonamide | | |
| 50 | N-[2,5-Bis(trifluoromethyl)benzyl]-4-{[(pyridin-3- | 533.1071 | 3, 86, 18 |
| | ylmethyl)carbamoyl]amino}benzenesulfonamide | | |
| 51 | N-(Biphenyl-2-yl)-2-[(4-{[(pyridin-3- | 516.2000 | 1, 2, 18 |
| | ylmethyl)carbamoyl]amino}phenyl)sulfamoyl]acetamide | | |
| 52 | 2′-[(4-Methylpiperazin-1-yl)methyl]-N-(4-{[(pyridin- | 639.2349 | 1, 2, 3, 4, 48 |
| | 3-ylmethyl)carbamoyl]amino}phenyl)-4- | | |
| | (trifluoromethyl)biphenyl-2-sulfonamide | | |
| 53 | 4-Nitro-N-(4-{[(pyridin-3- | 496.0914 | 1, 2, 3 |
| | ylmethyl)carbamoyl]amino}phenyl)-3- | | |
| | (trifluoromethyl)benzenesulfonamide | | |
| 54 | tert-Butyl 4-{2′-[(4-{[(pyridin-3- | 594.3085 | 37, 32, 18, 4 |
| | ylmethyl)carbamoyl]amino}phenoxy)methyl]biphenyl- | | |
| | 3-yl}piperazine-1-carboxylate | | |
| 55 | 2,4-dichloro-N-(4-{[(pyridin-3- | 451.0407 | 1, 2, 3 |
| | ylmethyl)carbamoyl]amino}phenyl)benzenesulfonamide | | |
| 56 | 1-(Pyridin-3-ylmethyl)-3-(4-{[2-(thiophen-3- | 416.1443 | 7, 8, 4 |
| | yl)phenoxy]methyl}phenyl)urea | | |
| 57 | 1-[4-(biphenyl-2-ylmethoxy)phenyl]-3-(pyridin-3- | 410.1886 | 10, 18 |
| | ylmethyl)urea | | |
| 58 | 2-chloro-N-(4-{[(pyridin-3- | 417.0875 | 1, 2, 3 |
| | ylmethyl)carbamoyl]amino}phenyl)benzenesulfonamide | | |
| 59 | 1-(4-{[2-(1-Ethyl-1H-pyrazol-4- | 428.2076 | 7, 8, 4 |
| | yl)phenoxy]methyl}phenyl)-3-(pyridin-3- | | |
| | ylmethyl)urea | | |
| 60 | 1-(4-{[2-(Pyridin-2-yl)benzyl]oxy}phenyl)-3- | 411.1811 | 37, 32, 18, 90, |
| | (pyridin-3-ylmethyl)urea | | 91 |
| 61 | 2′-Amino-N-(4-{[(pyridin-3- | 542.1576 | 1, 2, 3, 4 |
| | ylmethyl)carbamoyl]amino}phenyl)-4- | | |
| | (trifluoromethyl)biphenyl-2-sulfonamide | | |
| 62 | 1-[7-(Biphenyl-2-yloxy)heptyl]-3-pyridin-4-ylurea | 404.2305 | 46, 45, 44 |
| 63 | 3′-(Piperazin-1-yl)-N-(4-{[(pyridin-3- | 611.2060 | 1, 2, 3, 4, 32 |
| | ylmethyl)carbamoyl]amino}phenyl)-4- | | |
| | (trifluoromethyl)biphenyl-2-sulfonamide | | |
| 64 | 1-(4-{[(4′-Hydroxybiphenyl-2- | 426.1818 | 7, 8, 4 |
| | yl)oxy]methyl}phenyl)-3-(pyridin-3-ylmethyl)urea | | |
| 65 | 1-(4-{[2-(1,3,4-Oxadiazol-2- | 402.1551 | 7, 8 |
| | yl)phenoxy]methyl}phenyl)-3-(pyridin-3- | | |
| | ylmethyl)urea | | |
| 66 | 1-(4-{2-[3′-(Morpholin-4-yl)biphenyl-2- | 509.2571 | 10, 45, 46, 4 |
| | yl]ethoxy}phenyl)-3-(pyridin-3-ylmethyl)urea | | |
| 67 | N-(trans-4-{[(Pyridin-3- | 252.1395 | 18, 32, 3 |
| | ylmethyl)carbamoyl]amino}cyclohexyl)-2,5- | | |
| | bis(trifluoromethyl)benzenesulfonamide | | |
| 68 | N-(4-{[(Pyridin-3- | 451.1097 | 1, 2, 3 |
| | ylmethyl)carbamoyl]amino}phenyl)-2- | | |
| | (trifluoromethyl)benzenesulfonamide | | |
| 69 | N-(8-{[(Pyridin-3- | 495.2396 | 3, 68, 46 |
| | ylmethyl)carbamoyl]amino}octyl)biphenyl-2- | | |
| | sulfonamide | | |
| 70 | N-[2-Chloro-5-(trifluoromethyl)phenyl]-4-{[(pyridin- | 485.0654 | 5, 2, 18 |
| | 3-ylmethyl)carbamoyl]amino}benzenesulfonamide | | |
| 71 | 1-(4-{[2-(Morpholin-4-yl)phenoxy]methyl}phenyl)-3- | 419.2105 | 7, 8 |
| | (pyridin-3-ylmethyl)urea | | |
| 72 | N-(biphenyl-2-yl)-4-{[(pyridin-3- | 459.1473 | 5, 2, 6, 4 |
| | ylmethyl)carbamoyl]amino}benzenesulfonamide | | |
| 73 | 3′-[(2-Methylpyrrolidin-1-yl)methyl]-N-(4-{[(pyridin- | 624.2223 | 3, 53, 4, 48, |
| | 3-ylmethyl)carbamoyl]amino}phenyl)-4- | | 54, 18 |
| | (trifluoromethyl)biphenyl-2-sulfonamide | | |
| 74 | 1-[4-({2-[6-(Dimethylamino)pyridin-3- | 454.2237 | 37, 32, 18, 4 |
| | yl]benzyl}oxy)phenyl]-3-(pyridin-3-ylmethyl)urea | | |
| 75 | 1-(4-{[2-Bromo-5- | 496.0480 | 7, 8 |
| | (trifluoromethoxy)phenoxy]methyl}phenyl)-3- | | |
| | (pyridin-3-ylmethyl)urea | | |
| 76 | 1-[4-({[3′-(Piperidin-1-ylmethyl)biphenyl-2- | 507.3042 | 35, 4, 18, 32, |
| | yl]oxy}methyl)phenyl]-3-(pyridin-3-ylmethyl)urea | | 37 |
| 77 | 3-(Piperidin-1-yl)-N-{2′-[(4-{[(pyridin-3- | 681.2444 | 1, 2, 3, 4, 52, 9 |
| | ylmethyl)carbamoyl]amino}phenyl)sulfamoyl]-4′- | | |
| | (trifluoromethyl)biphenyl-3-yl}propanamide | | |
| 78 | N-(Biphenyl-2-yl)-2-(4-{[(pyridin-3- | 453.1943 | 40, 10, 18 |
| | ylmethyl)carbamoyl]amino}phenoxy)acetamide | | |
| 79 | N-(6-{[(Pyridin-3- | 467.2120 | 18, 32, 3 |
| | ylmethyl)carbamoyl]amino}hexyl)biphenyl-2- | | |
| | sulfonamide | | |
| 80 | 2-(2-Methyl-1H-imidazol-1-yl)-N-(4-{[(pyridin-3- | 531.1383 | 17, 18, 76 |
| | ylmethyl)carbamoyl]amino}phenyl)-5- | | |
| | (trifluoromethyl)benzenesulfonamide | | |
| 81 | 2′-[2-(4-{[(Pyridin-3- | 600.2963 [M + Na] | 10, 45, 46, 4, |
| | ylmethyl)carbamoyl]amino}phenoxy)ethyl]-N-[3- | | 40 |
| | (pyrrolidin-1-yl)propyl]biphenyl-2-carboxamide | | |
| 82 | 1-{4-[(2-{1-[2-(Morpholin-4-yl)ethyl]-1H-pyrazol-4- | 512.2534 | 37, 32, 18, 4 |
| | yl}benzyl)oxy]phenyl}-3-(pyridin-3-ylmethyl)urea | | |
| 83 | 1-(4-{[2-Bromo-4- | 496.0480 | 7, 8 |
| | (trifluoromethoxy)phenoxy]methyl}phenyl)-3- | | |
| | (pyridin-3-ylmethyl)urea | | |
| 84 | N-[2-Chloro-5-(trifluoromethyl)benzyl]-4-{[(pyridin- | 499.0800 | 3, 86, 18 |
| | 3-ylmethyl)carbamoyl]amino}benzenesulfonamide | | |
| 85 | 1-[4-({[3′-(Dimethylamino)biphenyl-2- | 453.2239 | 44, 20, 46, 4 |
| | yl]oxy}methyl)phenyl]-3-(pyridin-3-ylmethyl)urea | | |
| 86 | 1-(4-{[(5-Hydroxybiphenyl-2-yl)oxy]methyl}phenyl)- | 426.1812 | 70, 44, 4, 45, |
| | 3-(pyridin-3-ylmethyl)urea | | 46 |
| 87 | 1-[4-({2-[2-(Morpholin-4-yl)pyridin-4- | 496.2368 | 37, 32, 18, 4, |
| | yl]benzyl}oxy)phenyl]-3-(pyridin-3-ylmethyl)urea | | 32, 89 |
| 88 | 2-(1H-Pyrazol-5-yl)-N-(4-{[(pyridin-3- | 449.1299 | 1, 2, 3, 4 |
| | ylmethyl)carbamoyl]amino}phenyl)benzenesulfonamide | | |
| 89 | N-Cyclopropyl-2′-[2-(4-{[(pyridin-3- | 507.2404 | 10, 45, 46, 4, |
| | ylmethyl)carbamoyl]amino}phenoxy)ethyl]biphenyl- | | 40 |
| | 2-carboxamide | | |
| 90 | 3-Bromo-N-(4-{[(pyridin-3- | 463.0220 | 1, 2, 3 |
| | ylmethyl)carbamoyl]amino}phenyl)benzenesulfonamide | | |
| 91 | 1-[6-(4-Chlorophenoxy)hexyl]-3-pyridin-4-ylurea | 348.1541 | 10, 18 |
| 92 | N-(trans-4-{[(Pyridin-3- | 465.1968 | 3, 32, 46 |
| | ylmethyl)carbamoyl]amino}cyclohexyl)biphenyl-2- | | |
| | sulfonamide | | |
| 93 | N-(4-{[(pyridin-3- | 439.1794 | 17, 18 |
| | ylmethyl)carbamoyl]amino}butyl)biphenyl-2- | | |
| | sulfonamide | | |
| 94 | 1-{4-[(biphenyl-2-yloxy)methyl]benzyl}-3-pyridin-4- | 410.1970 | 10, 18 |
| | ylurea | | |
| 95 | 1-{4-[(2-Methyl-4-phenyl-7,8-dihydropyrido[4,3- | 479.2190 | 30, 49, 50, 32, |
| | d]pyrimidin-6(5H)-yl)carbonyl]phenyl}-3-(pyridin-3- | | 33, 34 |
| | ylmethyl)urea | | |
| 96 | N-(4-{[(Pyridin-3- | 465.1324 | 1, 2, 3 |
| | ylmethyl)carbamoyl]amino}phenyl)-1-[3- | | |
| | (trifluoromethyl)phenyl]methanesulfonamide | | |
| 97 | 1-{4-[1-(Biphenyl-2-yloxy)cyclopropyl]phenyl}-3- | 436.2073 | 36, 10, 18 |
| | (pyridin-3-ylmethyl)urea | | |
| 98 | 2-chloro-N-(4-{[(pyridin-3- | 485.1416 | 1, 2, 3 |
| | ylmethyl)carbamoyl]amino}phenyl)-5- | | |
| | (trifluoromethyl)benzenesulfonamide | | |
| 99 | 2-(Biphenyl-2-yloxy)-N-{5-[(pyridin-4- | 433.2324 | 10, 15, 39, 18 |
| | ylcarbamoyl)amino]pentyl}acetamide | | |
| 100 | 1-{4-[2-(Biphenyl-2-yl)ethyl]phenyl}-3-(pyridin-3- | 408.2051 | 7, 18, 2 |
| | ylmethyl)urea | | |
| 101 | 1-[5-(biphenyl-2-yloxy)pentyl]-3-pyridin-4-ylurea | 376.2066 | 10, 18 |
| 102 | 1-(Pyridin-3-ylmethyl)-3-(4-{[2-(pyridin-3- | 411.1815 | 7, 8, 4 |
| | yl)phenoxy]methyl}phenyl)urea | | |
| 103 | N-(4-{[(Pyridin-3- | 451.1050 | 1, 2, 3 |
| | ylmethyl)carbamoyl]amino}phenyl)-3- | | |
| | (trifluoromethyl)benzenesulfonamide | | |
| 104 | N-[2,5-Bis(trifluoromethyl)phenyl]-4-{[(pyridin-3- | 519.0918 | 5, 60, 18 |
| | ylmethyl)carbamoyl]amino}benzenesulfonamide | | |
| 105 | 3-(6-{[(4-{[(Pyridin-3- | 552.1359 | 1, 2, 3, 4 |
| | ylmethyl)carbamoyl]amino}phenyl)sulfamoyl]methyl}cyclohexa- | | |
| | 2,4-dien-1-yl)benzenesulfonamide | | |
| 106 | 2-(1H-Imidazol-1-yl)-N-{2′-[2-(4-{[(pyridin-3- | 547.2443 | 10, 45, 46, 4, |
| | ylmethyl)carbamoyl]amino}phenoxy)ethyl]biphenyl- | | 40 |
| | 2-yl}acetamide | | |
| 107 | 3-(4-Methylpiperazin-1-yl)-N-{2′-[2-(4-{[(pyridin-3- | 593.3238 | 10, 45, 46, 4, |
| | ylmethyl)carbamoyl]amino}phenoxy)ethyl]biphenyl- | | 85 |
| | 2-yl}propanamide | | |
| 108 | 1-[4-(2-{2′-[(4-Methylpiperazin-1- | 536.3134 | 10, 45, 46, 4, |
| | yl)methyl]biphenyl-2-yl}ethoxy)phenyl]-3-(pyridin- | | 35 |
| | 3-ylmethyl)urea | | |
| 109 | 1-{4-[2-(2-Methyl-4-phenyl-7,8-dihydropyrido[4,3- | 493.2341 | 30, 49, 50, 32, |
| | d]pyrimidin-6(5H)-yl)-2-oxoethyl]phenyl}-3- | | 33, 34 |
| | (pyridin-3-ylmethyl)urea | | |
| 110 | N,N-Dimethyl-2′-[2-(4-{[(pyridin-3- | 495.2403 | 10, 45, 46, 4, |
| | ylmethyl)carbamoyl]amino}phenoxy)ethyl]biphenyl- | | 40 |
| | 3-carboxamide | | |
| 111 | N~2~-Biphenyl-2-yl-N-(4-{[(pyridin-3- | 452.2080 | 79, 15, 40, 32, |
| | ylmethyl)carbamoyl]amino}phenyl)glycinamide | | 18 |
| 112 | 1-(Pyridin-3-ylmethyl)-3-(4-{2-[2′-(pyrrolidin-1- | 521.2574 | 10, 45, 46, 4, |
| | ylcarbonyl)biphenyl-2-yl]ethoxy}phenyl)urea | | 40 |
| 113 | 3′-[(4-Methylpiperazin-1-yl)methyl]-N-(4-{[(pyridin- | 639.2342 | 3, 53, 4, 48, |
| | 3-ylmethyl)carbamoyl]amino}phenyl)-4- | | 54, 18 |
| | (trifluoromethyl)biphenyl-2-sulfonamide | | |
| 114 | N-{2-[(4-{[(Pyridin-3- | 530.1907 | 1, 2, 3, 42, 9 |
| | ylmethyl)carbamoyl]amino}phenyl)sulfamoyl]ethyl}biphenyl- | | |
| | 2-carboxamide | | |
| 115 | 1-(4-{[(3-Bromopyridin-2-yl)oxy]methyl}phenyl)-3- | 415.0640 | 7, 8 |
| | (pyridin-3-ylmethyl)urea | | |
| 116 | N-{4-[(Pyridin-3-ylcarbamoyl)amino]benzyl}-2,5- | no MS data | 46, 3 |
| | bis(trifluoromethyl)benzenesulfonamide | | |
| 117 | 2′-[(4-{[(Pyridin-3- | 489.9195 | 44, 20, 46, 4 |
| | ylmethyl)carbamoyl]amino}benzyl)oxy]biphenyl-4- | | |
| | sulfonamide | | |
| 118 | 1-[4-(Biphenyl-2-ylethynyl)phenyl]-3-(pyridin-3- | 404.1764 | 55, 18 |
| | ylmethyl)urea | | |
| 119 | 1-(4-{[2-(2-Aminopyridin-4-yl)benzyl]oxy}phenyl)- | 426.1930 | 37, 32, 18, 90, |
| | 3-(pyridin-3-ylmethyl)urea | | 91 |
| 120 | 1-(4-{[2-(2-Methoxypyridin-4- | 441.1927 | 37, 32, 18, 90, |
| | yl)benzyl]oxy}phenyl)-3-(pyridin-3-ylmethyl)urea | | 91 |
| 121 | N-(4-{[(Pyridin-3- | 437.1944 | 18, 32, 33 |
| | ylmethyl)carbamoyl]amino}benzyl)biphenyl-2- | | |
| | carboxamide | | |
| 122 | N-{2′-[(4-{[(Pyridin-3- | 467.2041 | 44, 20, 46, 4 |
| | ylmethyl)carbamoyl]amino}benzyl)oxy]biphenyl-3- | | |
| | yl}acetamide | | |
| 123 | 2-bromo-4,6-dichloro-N-(4-{[(pyridin-3- | 528.9485 | 1, 2, 3 |
| | ylmethyl)carbamoyl]amino}phenyl)benzenesulfonamide | | |
| 124 | 1-[6-(biphenyl-2-yloxy)hexyl]-3-pyridin-4-ylurea | 390.2216 | 10, 18 |
| 125 | N-(5-{[(Pyridin-3- | 460.1419 | 18, 60, 3 |
| | ylmethyl)carbamoyl]amino}pyridin-2-yl)biphenyl-2- | | |
| | sulfonamide | | |
| 126 | 1-[7-(Biphenyl-2-yloxy)heptyl]-3-(pyridin-3- | 418.2491 | 46, 45, 44 |
| | ylmethyl)urea | | |
| 127 | 1-(4-{[(3′-{[(2R,6S)-2,6-Dimethylpiperidin-1- | 535.3074 | 44, 45, 46, 4, |
| | yl]methyl}biphenyl-2-yl)oxy]methyl}phenyl)-3- | | 48 |
| | (pyridin-3-ylmethyl)urea | | |
| 128 | 1-{4-[({3′-[(Dimethylamino)methyl]biphenyl-2- | 467.2413 | 44, 20, 46, 4, |
| | yl}oxy)methyl]phenyl}-3-(pyridin-3-ylmethyl)urea | | 48 |
| 129 | 1-(4-{[(2′,3′,4′,5′,6′-~2~H_5_)Biphenyl-2- | 415.2207 | 44, 20, 46, 4 |
| | yloxy]methyl}phenyl)-3-(pyridin-3-ylmethyl)urea | | |
| 130 | 1-(Pyridin-3-ylmethyl)-3-[4-({3′-[1-(pyrrolidin-1- | 507.2759 | 37, 32, 18, 4, |
| | yl)ethyl]biphenyl-2-yl}methoxy)phenyl]urea | | 51 |
| 131 | 1-(Pyridin-3-ylmethyl)-3-(4-{[2-(pyrimidin-5- | 412.1783 | 37, 32, 18, 90, |
| | yl)benzyl]oxy}phenyl)urea | | 91 |
| 132 | 3-bromo-N-(4-{[(pyridin-3- | 531.0207 | 1, 2, 3 |
| | ylmethyl)carbamoyl]amino}phenyl)-5- | | |
| | (trifluoromethyl)benzenesulfonamide | | |
| 133 | 1-{4-[(Biphenyl-2-yloxy)methyl]benzyl}-3-(pyridin- | 424.2011 | 79, 10, 18 |
| | 3-ylmethyl)urea | | |
| 134 | 2-(Morpholin-4-yl)-N-{2′-[2-(4-{[(pyridin-3- | 566.2952 | 10, 45, 46, 4, |
| | ylmethyl)carbamoyl]amino}phenoxy)ethyl]biphenyl- | | 40 |
| | 2-yl}acetamide | | |
| 135 | N~3~′,N~3~′-Dimethyl-N~2~-(4-{[(pyridin-3- | 566.1563 | 3, 53, 4, 54, 18 |
| | ylmethyl)carbamoyl]amino}phenyl)biphenyl-2,3′- | | |
| | disulfonamide | | |
| 136 | 1-(4-{[2-(Morpholin-4-yl)benzyl]oxy}phenyl)-3- | 419.2102 | 14, 56, 18 |
| | (pyridin-3-ylmethyl)urea | | |
| 137 | N-(5-{[(pyridin-3- | 453.2321 | 18, 32, 3 |
| | ylmethyl)carbamoyl]amino}pentyl)biphenyl-2- | | |
| | sulfonamide | | |
| 138 | 1-{4-[(Biphenyl-2-ylamino)methyl]phenyl}-3- | 409.2034 | 78, |
| | (pyridin-3-ylmethyl)urea | | |
| 139 | N-[4-({[(2-Methylpyridin-4- | 473.1654 | 17, 18 |
| | yl)carbamoyl]amino}methyl)phenyl]biphenyl-2- | | |
| | sulfonamide | | |
| 140 | N-(3-fluoro-4-{[(pyridin-3- | 477.1397 | 6, 2, 3, 4 |
| | ylmethyl)carbamoyl]amino}phenyl)biphenyl-2- | | |
| | sulfonamide | | |
| 141 | N-[2-(Morpholin-4-yl)ethyl]-2′-[2-(4-{[(pyridin-3- | 580.2923 | 10, 45, 46, 4, |
| | ylmethyl)carbamoyl]amino}phenoxy)ethyl]biphenyl- | | 40 |
| | 2-carboxamide | | |
| 142 | 1-{4-[2-(Biphenyl-2-yl)ethoxy]phenyl}-3-(pyridin-3- | 424.2028 | 10, 4, 18 |
| | ylmethyl)urea | | |
| 143 | 1-(2-Chlorophenyl)-N-(4-{[(pyridin-3- | 431.0932 | 1, 2, 3 |
| | ylmethyl)carbamoyl]amino}phenyl)methanesulfonamide | | |
| 144 | N-(Biphenyl-2-ylmethyl)-4-{[(pyridin-3- | 473.1630 | 3, 86, 18 |
| | ylmethyl)carbamoyl]amino}benzenesulfonamide | | |
| 145 | 1-{4-[(2-Bromo-3-fluorophenoxy)methyl]phenyl}-3- | 430.0546 | 7, 8 |
| | (pyridin-3-ylmethyl)urea | | |
| 146 | 1-(4-{2-[3′-(Dimethylamino)biphenyl-2- | 467.2463 | 10, 45, 46, 4 |
| | yl]ethoxy}phenyl)-3-(pyridin-3-ylmethyl)urea | | |
| 147 | 1-(Biphenyl-2-yl)-N-(4-{[(pyridin-3- | 473.1712 | 1, 2, 3, 4 |
| | ylmethyl)carbamoyl]amino}phenyl)methanesulfonamide | | |
| 148 | N-(4-{[(Pyridin-3- | 473.1663 | 18, 32, 3 |
| | ylmethyl)carbamoyl]amino}benzyl)biphenyl-2- | | |
| | sulfonamide | | |
| 149 | N-(4-{[(Pyridin-3- | 483.1250 | 1, 2, 9 |
| | ylmethyl)carbamoyl]amino}phenyl)-2,5- | | |
| | bis(trifluoromethyl)benzamide | | |
| 150 | 2′-chloro-N-(4-{[(pyridin-3- | 493.1099 | 1, 2, 3, 4 |
| | ylmethyl)carbamoyl]amino}phenyl)biphenyl-2- | | |
| | sulfonamide | | |
| 151 | 1-(4-{[(4-Fluorobiphenyl-2-yl)oxy]methyl}phenyl)-3- | 428.1730 | 7, 8, 4 |
| | (pyridin-3-ylmethyl)urea | | |
| 152 | N-(3-{[(pyridin-3- | 425.1636 | 17, 18 |
| | ylmethyl)carbamoyl]amino}propyl)biphenyl-2- | | |
| | sulfonamide | | |
| 153 | 1-{4-[Bis(biphenyl-2-ylmethyl)amino]phenyl}-3- | 575.2872 | 79, 32, 18 |
| | (pyridin-3-ylmethyl)urea | | |
| 154 | 2′-[2-(4-{[(Pyridin-3- | 503.1743 | 10, 45, 46, 4 |
| | ylmethyl)carbamoyl]amino}phenoxy)ethyl]biphenyl- | | |
| | 3-sulfonamide | | |
| 155 | 1-[(6-Aminopyridin-3-yl)methyl]-3-{4-[(biphenyl-2- | 425.2221 | 8, 33, 46 |
| | yloxy)methyl]phenyl}urea | | |
| 156 | 1-(4-{[(4-Hydroxybiphenyl-2-yl)oxy]methyl}phenyl)- | 426.1821 | 70, 44, 4, 45, |
| | 3-(pyridin-3-ylmethyl)urea | | 46 |
| 157 | 1-{4-[(biphenyl-2-ylsulfonyl)methyl]phenyl}-3- | 458.1536 | 11, 4, 12, 18 |
| | (pyridin-3-ylmethyl)urea | | |
| 158 | 1-(4-{[2-(4-Methyl-1H-imidazol-1- | 414.1931 | 44, 32, 18, 76 |
| | yl)benzyl]oxy}phenyl)-3-(pyridin-3-ylmethyl)urea | | |
| 159 | 1-{4-[(2-Methyl-4-phenyl-7,8-dihydropyrido[4,3- | 465.2390 | 30, 48, 49, 50, |
| | d]pyrimidin-6(5H)-yl)methyl]phenyl}-3-(pyridin-3- | | 52, 18 |
| | ylmethyl)urea | | |
| 160 | 1-[4-(2-{2′-[(4-Methylpiperazin-1- | 572.2647 [M + Na] | 10, 45, 46, 4, |
| | yl)carbonyl]biphenyl-2-yl}ethoxy)phenyl]-3-(pyridin- | | 40 |
| | 3-ylmethyl)urea | | |
| 161 | 4-Chloro-N-(4-{[(pyridin-4- | 417.0786 | 17, 18 |
| | ylcarbamoyl)amino]methyl}phenyl)benzenesulfonamide | | |
| 162 | 2,3-dichloro-N-(4-{[(pyridin-3- | 451.0402 | 1, 2, 3 |
| | ylmethyl)carbamoyl]amino}phenyl)benzenesulfonamide | | |
| 163 | 3-(4-Methylpiperazin-1-yl)-N-{2′-[2-(4-{[(pyridin-3- | 593.3218 | 10, 45, 46, 4, |
| | ylmethyl)carbamoyl]amino}phenoxy)ethyl]biphenyl- | | 85 |
| | 3-yl}propanamide | | |
| 164 | 1-(4-{[2-Chloro-5- | 436.1065 | 7, 8 |
| | (trifluoromethyl)phenoxy]methyl}phenyl)-3-(pyridin- | | |
| | 3-ylmethyl)urea | | |
| 165 | 5-(Dimethylamino)-N-(4-{[(pyridin-3- | 476.1743 | 1, 2, 3 |
| | ylmethyl)carbamoyl]amino}phenyl)naphthalene-1- | | |
| | sulfonamide | | |
| 166 | 2-(Morpholin-4-yl)-N-{2′-[2-(4-{[(pyridin-3- | 566.2952 | 10, 45, 46, 4, |
| | ylmethyl)carbamoyl]amino}phenoxy)ethyl]biphenyl- | | 40 |
| | 2-yl}acetamide | | |
| 167 | 2-(Biphenyl-2-yloxy)-N-(4-{[(pyridin-3- | 453.1931 | 10, 15, 39, 18 |
| | ylmethyl)carbamoyl]amino}phenyl)acetamide | | |
| 168 | 3′-[1-(4-Methylpiperazin-1-yl)ethyl]-N-(4-{[(pyridin- | 653.2508 | 3, 53, 4, 51, |
| | 3-ylmethyl)carbamoyl]amino}phenyl)-4- | | 54, 18 |
| | (trifluoromethyl)biphenyl-2-sulfonamide | | |
| 169 | 3-Methoxy-N-(4-{[(pyridin-3- | 413.1123 | 1, 2, 3 |
| | ylmethyl)carbamoyl]amino}phenyl)benzenesulfonamide | | |
| 170 | 1-(4-{[(6-Fluorobiphenyl-2-yl)oxy]methyl}phenyl)-3- | 428.1750 | 7, 8, 4 |
| | (pyridin-3-ylmethyl)urea | | |
| 171 | 2-(Biphenyl-2-yloxy)-N-{4-[(pyridin-4- | 419.2172 | 10, 15, 39, 18 |
| | ylcarbamoyl)amino]butyl}acetamide | | |
| 172 | 1-(4-{[2-(2-Fluoropyridin-3-yl)benzyl]oxy}phenyl)- | 429.1866 | 37, 32, 18, 4 |
| | 3-(pyridin-3-ylmethyl)urea | | |
| 173 | 1-[4-(2-{3′-[(4-Methylpiperazin-1- | 550.2822 | 10, 45, 46, 4, |
| | yl)carbonyl]biphenyl-2-yl}ethoxy)phenyl]-3-(pyridin- | | 40 |
| | 3-ylmethyl)urea | | |
| 174 | N-{2′-[2-(4-{[(Pyridin-3- | 481.2259 | 10, 45, 46, 4 |
| | ylmethyl)carbamoyl]amino}phenoxy)ethyl]biphenyl- | | |
| | 3-yl}acetamide | | |
| 175 | N-Ethyl-2′-[2-(4-{[(pyridin-3- | 495.2370 | 10, 45, 46, 4, |
| | ylmethyl)carbamoyl]amino}phenoxy)ethyl]biphenyl- | | 40 |
| | 3-carboxamide | | |
| 176 | 1-(Pyridin-3-ylmethyl)-3-[4-({[5- | 478.1737 | 7, 8, 4 |
| | (trifluoromethyl)biphenyl-2- | | |
| | yl]oxy}methyl)phenyl]urea | | |
| 177 | 1-[5-(Biphenyl-2-yloxy)pentyl]-3-(pyridin-3- | 390.2245 | 10, 18 |
| | ylmethyl)urea | | |
| 178 | N-(Biphenyl-2-ylmethyl)-4-{[(pyridin-3- | 437.1937 | 40, 32, 18 |
| | ylmethyl)carbamoyl]amino}benzamide | | |
| 179 | N-(biphenyl-2-yl)-4-{[(pyridin-3- | 423.1849 | 9, 2, 18 |
| | ylmethyl)carbamoyl]amino}benzamide | | |
| 180 | N-[2-(Morpholin-4-yl)ethyl]-2′-[2-(4-{[(pyridin-3- | 580.2923 | 10, 45, 46, 4, |
| | ylmethyl)carbamoyl]amino}phenoxy)ethyl]biphenyl- | | 40 |
| | 2-carboxamide | | |
| 181 | N-[2-(Piperidin-1-yl)ethyl]-2′-[2-(4-{[(pyridin-3- | 578.3247 | 10, 45, 46, 4, |
| | ylmethyl)carbamoyl]amino}phenoxy)ethyl]biphenyl- | | 40 |
| | 2-carboxamide | | |
| 182 | 4′-chloro-N-(4-{[(pyridin-3- | 493.1107 | 1, 2, 3, 4 |
| | ylmethyl)carbamoyl]amino}phenyl)biphenyl-2- | | |
| | sulfonamide | | |
| 183 | 1-[4-({3′-[1-(Cyclopropylamino)ethyl]biphenyl-2- | 493.2607 | 37, 32, 18, 4, |
| | yl}methoxy)phenyl]-3-(pyridin-3-ylmethyl)urea | | 51 |
| 184 | 1-(4-{[(3′-Hydroxybiphenyl-2- | 426.1815 | 7, 8, 4 |
| | yl)oxy]methyl}phenyl)-3-(pyridin-3-ylmethyl)urea | | |
| 185 | 5-fluoro-N-(4-{[(pyridin-3- | 477.1386 | 1, 2, 3, 4 |
| | ylmethyl)carbamoyl]amino}phenyl)biphenyl-2- | | |
| | sulfonamide | | |
| 186 | 1-[4-({3′-[(4-Methylpiperazin-1- | 536.2906 | 37, 32, 18, 4 |
| | yl)carbonyl]biphenyl-2-yl}methoxy)phenyl]-3- | | |
| | (pyridin-3-ylmethyl)urea | | |
| 187 | 2-(Biphenyl-2-yloxy)-N-(5-{[(pyridin-3- | 447.2399 | 10, 15, 39, 18 |
| | ylmethyl)carbamoyl]amino}pentyl)acetamide | | |
| 188 | 1-(Pyridin-3-ylmethyl)-3-(4-{[2-(pyrimidin-2- | 412.1757 | 37, 32, 18, 90, |
| | yl)benzyl]oxy}phenyl)urea | | 91 |
| 189 | 1-(4-{[2-(2-Aminopyrimidin-5- | 427.1864 | 37, 32, 18, 4 |
| | yl)benzyl]oxy}phenyl)-3-(pyridin-3-ylmethyl)urea | | |
| 190 | N-(4-{[(Pyridin-3- | 459.1486 | 1, 2, 3, 4 |
| | ylmethyl)carbamoyl]amino}phenyl)biphenyl-2- | | |
| | sulfonamide | | |
| 191 | 1-(4-{[(5-Chlorobiphenyl-2-yl)oxy]methyl}phenyl)- | 444.1476 | 7, 8, 4 |
| | 3-(pyridin-3-ylmethyl)urea | | |
| 192 | 1-[6-(3-Fluorophenyl)cyclohexa-2,4-dien-1-yl]-N-(4- | 491.1618 | 5, 18, 4 |
| | {[(pyridin-3- | | |
| | ylmethyl)carbamoyl]amino}phenyl)methanesulfonamide | | |
| 193 | N-[2-(Diethylamino)ethyl]-2′-[2-(4-{[(pyridin-3- | 566.3426 | 10, 45, 46, 4, |
| | ylmethyl)carbamoyl]amino}phenoxy)ethyl]biphenyl- | | 40 |
| | 2-carboxamide | | |
| 194 | 1-(4-{[2-(2-Fluoropyridin-4-yl)benzyl]oxy}phenyl)- | 429.1753 | 37, 32, 18, 4 |
| | 3-(pyridin-3-ylmethyl)urea | | |
| 195 | 1-{4-[(biphenyl-2-yloxy)methyl]benzyl}-3-pyridin-3- | 410.1984 | 10, 18 |
| | ylurea | | |
| 196 | 1-{4-[({3′-[(4-Methylpiperazin-1-yl)methyl]biphenyl- | 522.2895 | 44, 20, 46, 4, |
| | 2-yl}oxy)methyl]phenyl}-3-(pyridin-3-ylmethyl)urea | | 48 |
| 197 | 2-Chloro-N-(4-{[(pyridin-4- | 485.0807 | 17, 18 |
| | ylcarbamoyl)amino]methyl}phenyl)-5- | | |
| | (trifluoromethyl)benzenesulfonamide | | |
| 198 | 1-{4-[(Biphenyl-2-yloxy)methyl]phenyl}-3-{[6- | 478.1737 | 20, 45, 46 |
| | (trifluoromethyl)pyridin-3-yl]methyl}urea | | |
| 199 | 1-[4-({3′-[(2-Methylpyrrolidin-1-yl)methyl]biphenyl- | 507.2724 | 37, 32, 18, 4, |
| | 2-yl}methoxy)phenyl]-3-(pyridin-3-ylmethyl)urea | | 51 |
| 200 | 1-(4-{2-[2-(4-Methyl-1H-imidazol-1- | 428.2031 | 77, 2, 18, 76 |
| | yl)phenyl]ethoxy}phenyl)-3-(pyridin-3-ylmethyl)urea | | |
| 201 | 4′-chloro-3′-fluoro-N-(4-{[(pyridin-3- | 511.1061 | 1, 2, 3, 4 |
| | ylmethyl)carbamoyl]amino}phenyl)biphenyl-2- | | |
| | sulfonamide | | |
| 202 | 1-(4-{[(Biphenyl-2-ylmethyl)amino]methyl}phenyl)- | 423.2220 | 18, 32, 35 |
| | 3-(pyridin-3-ylmethyl)urea | | |
| 203 | 1-{4-[(2-bromophenoxy)methyl]phenyl}-3-(pyridin- | 412.0676 | 7, 8 |
| | 3-ylmethyl)urea | | |
| 204 | 1-Amino-3-{[({4-[(biphenyl-2- | 415.2148 | 7, 8, 62, 63, |
| | yloxy)methyl]phenyl}carbamoyl)amino]methyl}pyridinium | | |
| 205 | 2-bromo-N-(3-fluoro-4-{[(pyridin-3- | 479.0181 | 6, 2, 3 |
| | ylmethyl)carbamoyl]amino}phenyl)benzenesulfonamide | | |
| 206 | 1-[6-(Biphenyl-2-yloxy)hexyl]-3-pyridin-3-ylurea | 390.2267 | 10, 18 |
| 207 | 1-{4-[2-(Biphenyl-2-yloxy)ethyl]phenyl}-3-(pyridin- | 424.2009 | 44, 4, 2, 18 |
| | 3-ylmethyl)urea | | |
| 208 | 1-{4-[(8-Bromo-3,4-dihydroisoquinolin-2(1H)- | 453.1098 | 48, 52, 18 |
| | yl)methyl]phenyl}-3-(pyridin-3-ylmethyl)urea | | |
| 209 | 2,5-Difluoro-N-(4-{[(pyridin-3- | 419.0988 | 1, 2, 3 |
| | ylmethyl)carbamoyl]amino}phenyl)benzenesulfonamide | | |
| 210 | 1-{4-[(biphenyl-2-ylsulfinyl)methyl]phenyl}-3- | 442.1510 | 11, 4, 12, 18 |
| | (pyridin-3-ylmethyl)urea | | |
| 211 | 1-{4-[2-(Biphenyl-2-yl)ethoxy]phenyl}-3-pyridin-4- | 410.1871 | 10, 4, 18 |
| | ylurea | | |
| 212 | N-(4-{[(3- | 473.1613 | 17, 18 |
| | aminobenzyl)carbamoyl]amino}phenyl)biphenyl-2- | | |
| | sulfonamide | | |
| 213 | N-{4-[(Pyridin-4-ylcarbamoyl)amino]benzyl}-2,5- | no MS data | 46, 3 |
| | bis(trifluoromethyl)benzenesulfonamide | | |
| 214 | 2-Bromo-N-(4-{[(pyridin-3- | 531.0087 | 1, 2, 3 |
| | ylmethyl)carbamoyl]amino}phenyl)-5- | | |
| | (trifluoromethyl)benzenesulfonamide | | |
| 215 | N-(4-{[(Pyridin-3- | 433.1318 | 1, 2, 3 |
| | ylmethyl)carbamoyl]amino}phenyl)naphthalene-2- | | |
| | sulfonamide | | |
| 216 | N-[2,5-Bis(trifluoromethyl)phenyl]-4-{[(pyridin-3- | 483.1250 | 1, 33, 61 |
| | ylmethyl)carbamoyl]amino}benzamide | | |
| 217 | 1-{4-[2-(2′-Aminobiphenyl-2-yl)ethoxy]phenyl}-3- | 439.2113 | 10, 45, 46, 4 |
| | (pyridin-3-ylmethyl)urea | | |
| 218 | 1-(4-{2-[2-(1H-Imidazol-1- | 414.1885 | 77, 2, 18, 76 |
| | yl)phenyl]ethoxy}phenyl)-3-(pyridin-3-ylmethyl)urea | | |
| 219 | 1-(4-{[3′-(Piperazin-1-ylcarbonyl)biphenyl-2- | 522.2573 | 37, 32, 18, 4, |
| | yl]methoxy}phenyl)-3-(pyridin-3-ylmethyl)urea | | 51 |
| 220 | 3-bromo-N-(4-{[(pyridin-3- | 468.9839 | 1, 2, 3 |
| | ylmethyl)carbamoyl]amino}phenyl)thiophene-2- | | |
| | sulfonamide | | |
| 221 | N-[2-(Diethylamino)ethyl]-2′-[2-(4-{[(pyridin-3- | 566.3141 | 10, 45, 46, 4, |
| | ylmethyl)carbamoyl]amino}phenoxy)ethyl]biphenyl- | | 40 |
| | 3-carboxamide | | |
| 222 | N-methyl-N-(4-{[(pyridin-3- | 473.1721 | 1, 2, 3, 4, 19 |
| | ylmethyl)carbamoyl]amino}phenyl)biphenyl-2- | | |
| | sulfonamide | | |
| 223 | 1-{4-[2-(Biphenyl-2-ylamino)ethyl]phenyl}-3- | 423.2193 | 57, 60, 18 |
| | (pyridin-3-ylmethyl)urea | | |
| 224 | 1-{4-[(3′-{[4-(2-Hydroxyethyl)piperazin-1- | 588.2605 (M + Na) | 37, 32, 18, 4, |
| | yl]carbonyl}biphenyl-2-yl)methoxy]phenyl}-3- | | 51 |
| | (pyridin-3-ylmethyl)urea | | |
| 225 | 3′-[(4-Methylpiperazin-1-yl)methyl]-N-(4-{[(pyridin- | 571.2507 | 3, 53, 4, 48, |
| | 3-ylmethyl)carbamoyl]amino}phenyl)biphenyl-2- | | 54, 18 |
| | sulfonamide | | |
| 226 | 1-[4-({[3′-(Morpholin-4-ylmethyl)biphenyl-2- | 509.2584 | 44, 20, 46, 4, |
| | yl]oxy}methyl)phenyl]-3-(pyridin-3-ylmethyl)urea | | 48 |
| 227 | 5-Bromo-2-methoxy-N-(4-{[(pyridin-3- | 491.0387 | 1, 2, 3 |
| | ylmethyl)carbamoyl]amino}phenyl)benzenesulfonamide | | |
| 228 | N-(naphthalen-1-yl)-4-{[(pyridin-3- | 433.1302 | 5, 2, 6 |
| | ylmethyl)carbamoyl]amino}benzenesulfonamide | | |
| 229 | 3,5-difluoro-N-(4-{[(pyridin-3- | 495.1283 | 1, 2, 3, 4 |
| | ylmethyl)carbamoyl]amino}phenyl)biphenyl-2- | | |
| | sulfonamide | | |
| 230 | 2-methyl-N-(4-{[(pyridin-3- | 363.1484 | 1, 2, 3 |
| | ylmethyl)carbamoyl]amino}phenyl)propane-1- | | |
| | sulfonamide | | |
| 231 | 2-(cyclohexylamino)-N-(4-{[(pyridin-3- | 480.2070 | 1, 2, 3, 22 |
| | ylmethyl)carbamoyl]amino}phenyl)benzenesulfonamide | | |
| 232 | 1-{trans-4-[(Biphenyl-2-yloxy)methyl]cyclohexyl}-3- | 416.2350 | 73, 18, 74, 10 |
| | (pyridin-3-ylmethyl)urea | | |
| 233 | 1-{4-[(2-Bromo-5-methoxyphenoxy)methyl]phenyl}- | 442.0759 | 7, 8 |
| | 3-(pyridin-3-ylmethyl)urea | | |
| 234 | 2′-[2-(4-{[(Pyridin-3- | 564.2943 | 10, 45, 46, 4, |
| | ylmethyl)carbamoyl]amino}phenoxy)ethyl]-N-[2- | | 40 |
| | (pyrrolidin-1-yl)ethyl]biphenyl-2-carboxamide | | |
| 235 | N-(Biphenyl-2-yl)-1-(4-{[(pyridin-3- | 473.1663 | 3, 2, 18 |
| | ylmethyl)carbamoyl]amino}phenyl)methanesulfonamide | | |
| 236 | 3′-fluoro-N-(4-{[(pyridin-3- | 477.1419 | 1, 2, 3, 4 |
| | ylmethyl)carbamoyl]amino}phenyl)biphenyl-2- | | |
| | sulfonamide | | |
| 237 | 2-(1H-Imidazol-1-yl)-N-(4-{[(pyridin-3- | 517.1229 | 17, 18, 76 |
| | ylmethyl)carbamoyl]amino}phenyl)-5- | | |
| | (trifluoromethyl)benzenesulfonamide | | |
| 238 | 1-{4-[(Biphenyl-2-ylmethyl)(3- | 479.2750 | 1, 2, 35, 35 |
| | methylbutyl)amino]phenyl}-3-(pyridin-3- | | |
| | ylmethyl)urea | | |
| 239 | 1-[(6-Aminopyridin-3-yl)methyl]-3-{4-[2-(biphenyl- | 439.2139 | 5, 18, 4 |
| | 2-yl)ethoxy]phenyl}urea | | |
| 240 | 4′-fluoro-N-(4-{[(pyridin-3- | 477.1404 | 1, 2, 3, 4 |
| | ylmethyl)carbamoyl]amino}phenyl)biphenyl-2- | | |
| | sulfonamide | | |
| 241 | 2-bromo-4,6-difluoro-N-(4-{[(pyridin-3- | 497.0093 | 1, 2, 3 |
| | ylmethyl)carbamoyl]amino}phenyl)benzenesulfonamide | | |
| 242 | 1-[4-(2-{2′-[2-(Morpholin-4-yl)ethoxy]biphenyl-2- | 553.2996 | 10, 45, 46, 4, |
| | yl}ethoxy)phenyl]-3-(pyridin-3-ylmethyl)urea | | 10 |
| 243 | 2-chloro-4,5-difluoro-N-(4-{[(pyridin-3- | 453.0638 | 1, 2, 3 |
| | ylmethyl)carbamoyl]amino}phenyl)benzenesulfonamide | | |
| 244 | 1-{4-[(biphenyl-2-ylmethyl)sulfanyl]phenyl}-3- | 426.1631 | 13, 2, 4, 18 |
| | (pyridin-3-ylmethyl)urea | | |
| 245 | 1-{trans-4-[(2-Phenylethyl)amino]cyclohexyl}-3- | 353.2384 | 35, 32, 46 |
| | (pyridin-3-ylmethyl)urea | | |
| 246 | N,N-Dimethyl-2′-[(4-{[(pyridin-3- | 517.1919 | 44, 20, 46, 4 |
| | ylmethyl)carbamoyl]amino}benzyl)oxy]biphenyl-3- | | |
| | sulfonamide | | |
| 247 | 1-{4-[(biphenyl-2-ylmethyl)sulfonyl]phenyl}-3- | 442.1615 | 13, 2, 4, 18, 12 |
| | (pyridin-3-ylmethyl)urea | | |
| 248 | N-(4-{[(Pyridin-3- | 624.2498 | 3, 53, 4, 51, |
| | ylmethyl)carbamoyl]amino}phenyl)-3′-[1-(pyrrolidin- | | 54, 18 |
| | 1-yl)ethyl]-4-(trifluoromethyl)biphenyl-2- | | |
| | sulfonamide | | |
| 249 | 1-[6-(biphenyl-2-yloxy)hexyl]-3-(pyridin-3- | 404.2339 | 10, 18 |
| | ylmethyl)urea | | |
| 250 | N-[3-(6-{[(4-{[(Pyridin-3- | 530.1901 | 5, 18, 4 |
| | ylmethyl)carbamoyl]amino}phenyl)sulfamoyl]methyl}cyclohexa- | | |
| | 2,4-dien-1-yl)phenyl]acetamide | | |
| 251 | 2-(pyridin-4-yl)-N-(4-{[(pyridin-3- | 460.1435 | 1, 2, 3, 4 |
| | ylmethyl)carbamoyl]amino}phenyl)benzenesulfonamide | | |
| 252 | 1-{4-[2-(2-Methyl-4-phenyl-7,8-dihydropyrido[4,3- | 479.2544 | 30, 49, 50, 32, |
| | d]pyrimidin-6(5H)-yl)ethyl]phenyl}-3-(pyridin-3- | | 37, 2, 18 |
| | ylmethyl)urea | | |
| 253 | 2-(2-Methyl-1H-imidazol-1-yl)-N-(4-{[(pyridin-3- | 463.1502 | 17, 18, 76 |
| | ylmethyl)carbamoyl]amino}phenyl)benzenesulfonamide | | |
| 254 | 1-(4-{[3′-(Piperazin-1-yl)biphenyl-2- | 494.2605 | 37, 32, 18, 4 |
| | yl]methoxy}phenyl)-3-(pyridin-3-ylmethyl)urea | | |
| 255 | 1-(4-{[2-(6-Fluoropyridin-3-yl)benzyl]oxy}phenyl)- | 429.1724 | 37, 32, 18, 90, |
| | 3-(pyridin-3-ylmethyl)urea | | 91 |
| 256 | N-(2-chlorophenyl)-4-{[(pyridin-3- | 417.0773 | 5, 2, 6 |
| | ylmethyl)carbamoyl]amino}benzenesulfonamide | | |
| 257 | 2-(Biphenyl-2-yloxy)-N-(4-{[(pyridin-3- | 433.2259 | 10, 20, 40, 18 |
| | ylmethyl)carbamoyl]amino}butyl)acetamide | | |
| 258 | 2-(4-Methyl-1H-imidazol-1-yl)-N-(4-{[(pyridin-3- | 531.1403 | 17, 18, 76 |
| | ylmethyl)carbamoyl]amino}phenyl)-5- | | |
| | (trifluoromethyl)benzenesulfonamide | | |
| 259 | N,N-Diethyl-2′-[2-(4-{[(pyridin-3- | 523.2704 | 10, 45, 46, 4, |
| | ylmethyl)carbamoyl]amino}phenoxy)ethyl]biphenyl- | | 40 |
| | 2-carboxamide | | |
| 260 | 2-(4-Methyl-1H-imidazol-1-yl)-N-(4-{[(pyridin-3- | 463.1523 | 17, 18, 76 |
| | ylmethyl)carbamoyl]amino}phenyl)benzenesulfonamide | | |
| 261 | 1-[4-({2-[2-(Piperazin-1-yl)pyridin-4- | 495.2477 | 37, 32, 18, 4, |
| | yl]benzyl}oxy)phenyl]-3-(pyridin-3-ylmethyl)urea | | 32 |
| 262 | N-(4-{[(Pyridin-3- | 497.1379 | 3, 53, 4, 48, |
| | ylmethyl)carbamoyl]amino}benzyl)-2,5- | | 54, 18 |
| | bis(trifluoromethyl)benzamide | | |
| 263 | 1-{4-[({2′-[(4-Methylpiperazin-1-yl)methyl]biphenyl- | 522.2843 | 44, 45, 46, 4, |
| | 2-yl}oxy)methyl]phenyl}-3-(pyridin-3-ylmethyl)urea | | 48 |
| 264 | 1-{4-[(2-{2-[4-(Propan-2-yl)piperazin-1-yl]pyridin-4- | 537.2946 | 37, 32, 18, 4, |
| | yl}benzyl)oxy]phenyl}-3-(pyridin-3-ylmethyl)urea | | 32, 88 |
| 265 | 3-chloro-N-(4-{[(pyridin-3- | 485.0793 | 1, 2, 3 |
| | ylmethyl)carbamoyl]amino}phenyl)-5- | | |
| | (trifluoromethyl)benzenesulfonamide | | |
| 266 | 1-{4-[2-Oxo-2-(2-phenylpiperidin-1- | 429.2280 | 18, 33, 34 |
| | yl)ethyl]phenyl}-3-(pyridin-3-ylmethyl)urea | | |
| 267 | 1-{4-[(E)-2-(Biphenyl-2-yl)ethenyl]phenyl}-3- | 406.1893 | 75, 18 |
| | (pyridin-3-ylmethyl)urea | | |
| 268 | 3′-(Dimethylamino)-N-(4-{[(pyridin-3- | 502.1871 | 3, 53, 4, 54, 18 |
| | ylmethyl)carbamoyl]amino}phenyl)biphenyl-2- | | |
| | sulfonamide | | |
| 269 | N-(2-Bromophenyl)-4-{[(pyridin-3- | | 5, 2, 18 |
| | ylmethyl)carbamoyl]amino}benzenesulfonamide | | |
| 270 | N-[2-(Piperidin-1-yl)ethyl]-2′-[2-(4-{[(pyridin-3- | 578.3247 | 10, 45, 46, 4, |
| | ylmethyl)carbamoyl]amino}phenoxy)ethyl]biphenyl- | | 40 |
| | 2-carboxamide | | |
| 271 | N-[4-({[(6-Aminopyridin-3- | 474.1585 | 8, 4, 32, 18 |
| | yl)methyl]carbamoyl}amino)phenyl]biphenyl-2- | | |
| | sulfonamide | | |
| 272 | N-[2-(Diethylamino)ethyl]-2′-[2-(4-{[(pyridin-3- | 566.3426 | 10, 45, 46, 4, |
| | ylmethyl)carbamoyl]amino}phenoxy)ethyl]biphenyl- | | 40 |
| | 2-carboxamide | | |
| 273 | 1-{4-[(2-{1-[2-(Morpholin-4-yl)ethyl]-1H-pyrazol-4- | 513.2705 | 37, 32, 18, 4 |
| | yl}benzyl)oxy]phenyl}-3-(pyridin-3-ylmethyl)urea | | |
| 274 | 1-(2-{4-[(Biphenyl-2-yloxy)methyl]-1H-1,2,3-triazol- | 429.1994 | 8, 83, 84, 32, 6 |
| | 1-yl}ethyl)-3-(pyridin-3-ylmethyl)urea | | |
| 275 | 2,3-Dimethyl-4-oxo-N-(4-{[(pyridin-3- | 479.1502 | 20, 21 |
| | ylmethyl)carbamoyl]amino}phenyl)-3,4- | | |
| | dihydroquinazoline-6-sulfonamide | | |
| 276 | 1-{4-[(Biphenyl-2-ylmethyl)(ethyl)amino]phenyl}-3- | 437.2348 | 1, 2, 35, 35 |
| | (pyridin-3-ylmethyl)urea | | |
| 277 | N-(4-{[(pyridin-3- | 519.1249 | 1, 2, 3 |
| | ylmethyl)carbamoyl]amino}phenyl)-2,5- | | |
| | bis(trifluoromethyl)benzenesulfonamide | | |
| 278 | 2-[3-(morpholin-4-yl)pyrrolidin-1-yl]-N-(4- | 537.2279 | 1, 2, 3, 22 |
| | {[(pyridin-3- | | |
| | ylmethyl)carbamoyl]amino}phenyl)benzenesulfonamide | | |
| 279 | 5-Chloro-2-methoxy-N-(4-{[(pyridin-3- | 447.0969 | 1, 2, 3 |
| | ylmethyl)carbamoyl]amino}phenyl)benzenesulfonamide | | |
| 280 | N-{2-[(4-{[(Pyridin-3- | 566.1529 | 1, 2, 3, 42, 3 |
| | ylmethyl)carbamoyl]amino}phenyl)sulfamoyl]ethyl}biphenyl- | | |
| | 2-sulfonamide | | |
| 281 | 4-tert-Butyl-N-(4-{[(pyridin-3- | 439.1781 | 1, 2, 3 |
| | ylmethyl)carbamoyl]amino}phenyl)benzenesulfonamide | | |
| 282 | 1-(4-{[(2′-Fluorobiphenyl-2-yl)oxy]methyl}phenyl)- | 428.1779 | 7, 8, 4 |
| | 3-(pyridin-3-ylmethyl)urea | | |
| 283 | 1-(Pyridin-3-ylmethyl)-3-(4-{[2-(pyrrolidin-1- | 403.2159 | 7, 8 |
| | yl)phenoxy]methyl}phenyl)urea | | |
| 284 | N,N-Dimethyl-2′-[2-(4-{[(pyridin-3- | 531.2042 | 10, 45, 46, 4 |
| | ylmethyl)carbamoyl]amino}phenoxy)ethyl]biphenyl- | | |
| | 2-sulfonamide | | |
| 285 | 1-(4-{[2-(Pyridin-3-yl)benzyl]oxy}phenyl)-3- | 411.1807 | 37, 32, 18, 4 |
| | (pyridin-3-ylmethyl)urea | | |
| 286 | 1-(4-{[2′,4′-Difluoro-3′-(pyrrolidin-1- | 529.2428 | 37, 32, 18, 4, |
| | ylmethyl)biphenyl-2-yl]methoxy}phenyl)-3-(pyridin- | | 51 |
| | 3-ylmethyl)urea | | |
| 287 | 2-(morpholin-4-yl)-N-(4-{[(pyridin-3- | 468.1706 | 1, 2, 3, 22 |
| | ylmethyl)carbamoyl]amino}phenyl)benzenesulfonamide | | |
| 288 | 3′-[(Dimethylamino)methyl]-N-(4-{[(pyridin-3- | 516.2094 | 3, 53, 4, 48, |
| | ylmethyl)carbamoyl]amino}phenyl)biphenyl-2- | | 54, 18 |
| | sulfonamide | | |
| 289 | 2-Bromo-N-(4-{[(pyridin-3- | 461.0250 | 1, 2, 3 |
| | ylmethyl)carbamoyl]amino}phenyl)benzenesulfonamide | | |
| 290 | 1-{4-[(biphenyl-2-ylsulfanyl)methyl]phenyl}-3- | 426.1648 | 11, 4, 18 |
| | (pyridin-3-ylmethyl)urea | | |
| 291 | 3′-(Morpholin-4-yl)-N-(4-{[(pyridin-3- | 544.2057 | 1, 2, 3, 4 |
| | ylmethyl)carbamoyl]amino}phenyl)biphenyl-2- | | |
| | sulfonamide | | |
| 292 | 1-(4-{2-[3′-(Morpholin-4-ylcarbonyl)biphenyl-2- | 537.2509 | 10, 45, 46, 4, |
| | yl]ethoxy}phenyl)-3-(pyridin-3-ylmethyl)urea | | 40 |
| 293 | 1-[6-(3-Hydroxyphenyl)cyclohexa-2,4-dien-1-yl]-N- | 489.1637 | 1, 2, 3 |
| | (4-{[(pyridin-3- | | |
| | ylmethyl)carbamoyl]amino}phenyl)methanesulfonamide | | |
| 294 | 1-{4-[2-(2′-Cyanobiphenyl-2-yl)ethoxy]phenyl}-3- | 449.1955 | 10, 45, 46, 4 |
| | (pyridin-3-ylmethyl)urea | | |
| 295 | 1-{4-[({6-[(4-Methylpiperazin-1-yl)methyl]biphenyl- | 522.2906 | 44, 20, 46, 4, |
| | 2-yl}oxy)methyl]phenyl}-3-(pyridin-3-ylmethyl)urea | | 48 |
| 296 | 1-{4-[(Biphenyl-2-yloxy)methyl]phenyl}-3-(pyridin- | 410.1867 | 10, 18 |
| | 3-ylmethyl)urea | | |
| 297 | N-(4-{[(pyridin-3- | 423.1842 | 1, 2, 9 |
| | ylmethyl)carbamoyl]amino}phenyl)biphenyl-2- | | |
| | carboxamide | | |
| 298 | N-(4-{[(Pyridin-3- | 610.2124 | 1, 2, 3, 4 |
| | ylmethyl)carbamoyl]amino}phenyl)-2′-(pyrrolidin-1- | | |
| | ylmethyl)-4-(trifluoromethyl)biphenyl-2-sulfonamide | | |
| 299 | 1-[4-({2′-[(2-Methylpyrrolidin-1-yl)methyl]biphenyl- | 507.2857 | 37, 32, 18, 4, |
| | 2-yl}methoxy)phenyl]-3-(pyridin-3-ylmethyl)urea | | 51 |
| 300 | 1-(4-{[(Biphenyl-2- | 437.2306 | 35, 72, 32, 18 |
| | ylmethyl)(methyl)amino]methyl}phenyl)-3-(pyridin- | | |
| | 3-ylmethyl)urea | | |
| 301 | 3-Biphenyl-2-yl-3-(4-{[(pyridin-3- | 438.1913 | 5, 18, 4 |
| | ylmethyl)carbamoyl]amino}phenyl)urea (non- | | |
| | preferred name) | | |
| 302 | 1-{4-[(Biphenyl-2-yloxy)methyl]phenyl}-3-[(6- | 444.1478 | 8, 33, 46 |
| | chloropyridin-3-yl)methyl]urea | | |
| 303 | 1-(Pyridin-3-ylmethyl)-3-[4-({[4- | 494.1687 | 7, 8, 4 |
| | (trifluoromethoxy)biphenyl-2- | | |
| | yl]oxy}methyl)phenyl]urea | | |
| 304 | 1-(4′-Fluorobiphenyl-2-yl)-N-(4-{[(pyridin-3- | 491.1652 | 5, 18, 4 |
| | ylmethyl)carbamoyl]amino}phenyl)methanesulfonamide | | |
| 305 | 3-methyl-N-(4-{[(pyridin-3- | 397.1325 | 1, 2, 3 |
| | ylmethyl)carbamoyl]amino}phenyl)benzenesulfonamide | | |
| 306 | 2-chloro-N-(4-{[(pyridin-3- | 485.0654 | 1, 2, 3 |
| | ylmethyl)carbamoyl]amino}phenyl)-4- | | |
| | (trifluoromethyl)benzenesulfonamide | | |
| 307 | 1-{4-[2-(3′-Aminobiphenyl-2-yl)ethoxy]phenyl}-3- | 439.2133 | 10, 45, 46, 4 |
| | (pyridin-3-ylmethyl)urea | | |
| 308 | 2-chloro-6-methyl-N-(4-{[(pyridin-3- | 431.1052 | 1, 2, 3 |
| | ylmethyl)carbamoyl]amino}phenyl)benzenesulfonamide | | |
| 309 | 2,5-dichloro-N-(4-{[(pyridin-3- | 451.0404 | 1, 2, 3 |
| | ylmethyl)carbamoyl]amino}phenyl)benzenesulfonamide | | |
| 310 | 1-{4-[(2,3-Dimethyl-4-oxo-3,5,7,8- | 419.2199 | 30, 31, 32, 48, |
| | tetrahydropyrido[4,3-d]pyrimidin-6(4H)- | | 52 |
| | yl)methyl]phenyl}-3-(pyridin-3-ylmethyl)urea | | |
| 311 | N-{4-[(4-Methylpiperazin-1-yl)methyl]phenyl}-4-[(4- | 614.2541 | 1, 2, 3, 9 |
| | {[(pyridin-3- | | |
| | ylmethyl)carbamoyl]amino}phenyl)sulfamoyl]benzamide | | |
| 312 | 1-[4-({2′-[(2,6-Dimethylpiperidin-1- | 535.3129 | 37, 32, 18, 4, |
| | yl)methyl]biphenyl-2-yl}methoxy)phenyl]-3-(pyridin- | | 51 |
| | 3-ylmethyl)urea | | |
| 313 | 1-(4-{[2,5-Bis(trifluoromethyl)benzyl]oxy}phenyl)-3- | 492.1137 | 8, 32, 18 |
| | (pyridin-3-ylmethyl)urea | | |
| 314 | 2-bromo-4-fluoro-N-(4-{[(pyridin-3- | 479.0190 | 1, 2, 3 |
| | ylmethyl)carbamoyl]amino}phenyl)benzenesulfonamide | | |
| 315 | 1-{4-[2-(3′-Cyanobiphenyl-2-yl)ethoxy]phenyl}-3- | 449.1990 | 10, 45, 46, 4 |
| | (pyridin-3-ylmethyl)urea | | |
| 316 | N-(4-{[(Pyridin-3- | 542.2240 | 3, 53, 4, 48, |
| | ylmethyl)carbamoyl]amino}phenyl)-3′-(pyrrolidin-1- | | 54, 18 |
| | ylmethyl)biphenyl-2-sulfonamide | | |
| 317 | 2-phenyl-N-(4-{[(pyridin-3- | 361.1650 | 1, 2, 9 |
| | ylmethyl)carbamoyl]amino}phenyl)acetamide | | |
| 318 | 1-[4-(2-{2′-[2-(Morpholin-4-yl)ethoxy]biphenyl-2- | 553.2996 | 10, 45, 46, 4, |
| | yl}ethoxy)phenyl]-3-(pyridin-3-ylmethyl)urea | | 10 |
| 319 | N~3~,N~3~-Diethyl-N-{2′-[(4-{[(pyridin-3- | 669.2635 | 1, 2, 3, 4, 52, 9 |
| | ylmethyl)carbamoyl]amino}phenyl)sulfamoyl]-4′- | | |
| | (trifluoromethyl)biphenyl-3-yl}-beta-alaninamide | | |
| 320 | N-[2-Bromo-5-(trifluoromethyl)phenyl]-4-{[(pyridin- | 529.0148 | 5, 60, 18 |
| | 3-ylmethyl)carbamoyl]amino}benzenesulfonamide | | |
| 321 | 2,5-Dimethoxy-N-(4-{[(pyridin-3- | 443.1372 | 1, 2, 3 |
| | ylmethyl)carbamoyl]amino}phenyl)benzenesulfonamide | | |
| 322 | N-(4-{[(pyridin-3- | 519.0908 | 1, 2, 3 |
| | ylmethyl)carbamoyl]amino}phenyl)-3,5- | | |
| | bis(trifluoromethyl)benzenesulfonamide | | |
| 323 | 2′-[2-(4-{[(Pyridin-3- | 600.2963 [M + Na] | 10, 45, 46, 4, |
| | ylmethyl)carbamoyl]amino}phenoxy)ethyl]-N-[3- | | 40 |
| | (pyrrolidin-1-yl)propyl]biphenyl-2-carboxamide | | |
| 324 | 1-(Pyridin-3-ylmethyl)-3-[4-({[5- | 494.1707 | 7, 8, 4 |
| | (trifluoromethoxy)biphenyl-2- | | |
| | yl]oxy}methyl)phenyl]urea | | |
| 325 | 2-(1-Ethyl-1H-pyrazol-4-yl)-N-(4-{[(pyridin-3- | 477.1685 | 1, 2, 3, 4 |
| | ylmethyl)carbamoyl]amino}phenyl)benzenesulfonamide | | |
| 326 | 1-{4-[(Biphenyl-2-ylmethyl)amino]phenyl}-3- | 409.2022 | 35, 18 |
| | (pyridin-3-ylmethyl)urea | | |
| 327 | 2-fluoro-N-(4-{[(pyridin-3- | 469.0828 | 1, 2, 3 |
| | ylmethyl)carbamoyl]amino}phenyl)-5- | | |
| | (trifluoromethyl)benzenesulfonamide | | |
| 328 | 1-{4-[(Biphenyl-2-yloxy)methyl]phenyl}-3-[(1- | 426.1802 | 44, 45, 46, 82 |
| | oxidopyridin-3-yl)methyl]urea | | |
| 329 | 2-bromo-N-(4-{[(pyridin-4- | 531.0054 | 17, 18 |
| | ylcarbamoyl)amino]methyl}phenyl)-5- | | |
| | (trifluoromethyl)benzenesulfonamide | | |
| 330 | 1-{4-[(Biphenyl-2-yloxy)methyl]phenyl}-3-[(1- | 413.1985 | 44, 20, 46 |
| | methyl-1H-pyrazol-4-yl)methyl]urea | | |
| 331 | 1-{4-[(2-Bromo-4,5- | 450.0418 | 7, 8 |
| | difluorophenoxy)methyl]phenyl}-3-(pyridin-3- | | |
| | ylmethyl)urea | | |

TABLE 2

| | | | HRMS | |
|---|---|---|---|---|
| | | | Found | Syn. |
| Ex. No. | Structure | IUPACName | [M + H]⁺ | Proc. |
| 332 | 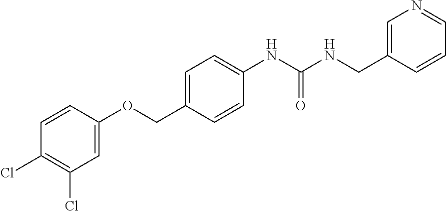 [SMILES: O=C(NCc1cccnc1)Nc1ccc(COc2ccc(Cl)c(Cl)c2)cc1] | 1-{4-[(3,4- Dichlorophenoxy) methyl]phenyl}-3- (pyridin-3- ylmethyl)urea | 402.0768 | 7, 8 |
| 333 | 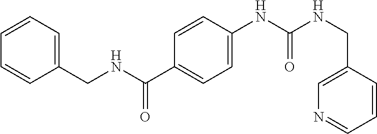 [SMILES: O=C(NCc1cccnc1)Nc1ccc(C(=O)NCc2ccccc2)cc1] | N-benzyl-4- {[(pyridin-3- ylmethyl)carbamoyl] amino}benzamide | 361.1666 | 1, 16, 9 |
| 334 | 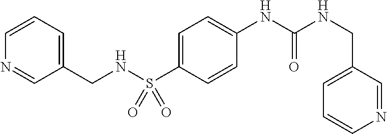 [SMILES: O=C(NCc1cccnc1)Nc1ccc(S(=O)(=O)NCc2cccnc2)cc1] | N-(pyridin-3- ylmethyl)-4- {[(pyridin-3- ylmethyl)carbamoyl] amino} benzenesulfonamide | 398.1290 | 27 |
| 335 | 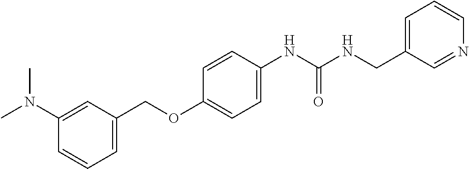 [SMILES: CN(C)c1cccc(COc2ccc(NC(=O)NCc3cccnc3)cc2)c1] | 1-(4-{[3- (Dimethylamino) benzyl]oxy}phenyl)-3- (pyridin-3- ylmethyl)urea | 402.1441 | 10, 56, 18 |
| 336 | 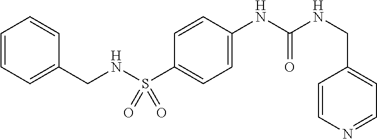 [SMILES: O=C(NCc1ccncc1)Nc1ccc(S(=O)(=O)NCc2ccccc2)cc1] | N-Benzyl-4- {[(pyridin-4- ylmethyl)carbamoyl] amino} benzenesulfonamide | 397.1369 | 5, 2, 18 |
| 337 | 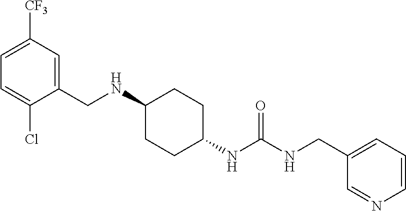 [SMILES: O=C(NCc1cccnc1)N[C@H]1CC[C@H](NCc2cc(C(F)(F)F)ccc2Cl)CC1] | 1-(trans-4-{[2- Chloro-5- (trifluoromethyl) benzyl]amino} cyclohexyl)-3-(pyridin-3- ylmethyl)urea | 441.1658 | 72, 32, 46 |
| 338 | 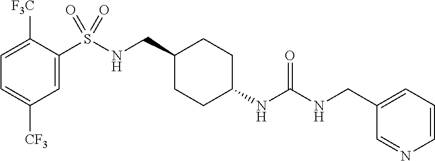 [SMILES: O=C(NCc1cccnc1)N[C@H]1CC[C@H](CNS(=O)(=O)c2cc(C(F)(F)F)ccc2C(F)(F)F)CC1] | N-[(trans-4- {[(Pyridin-3- ylmethyl)carbamoyl] amino}cyclohexyl) methyl]-2,5- bis(trifluoromethyl) benzenesulfonamide | 539.1543 | 3, 32, 46 |
| 339 | 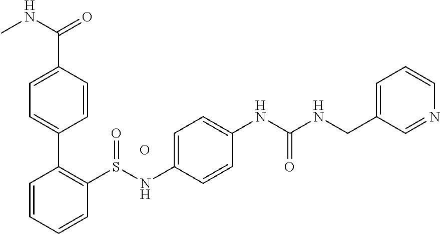 | N-Methyl-2′-[(4- {[(pyridin-3- ylmethyl)carbamoyl] amino}phenyl) sulfamoyl] biphenyl-4- carboxamide | 516.1756 | 1, 2, 3, 4 |
| 340 | 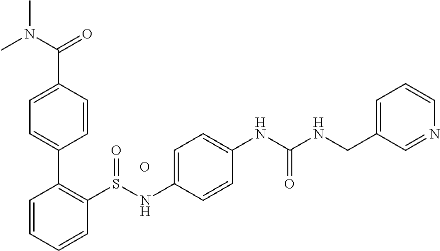 | N,N-Dimethyl-2′-[(4- {[(pyridin-3- ylmethyl)carbamoyl] amino}phenyl) sulfamoyl]biphenyl-4- carboxamide | 530.1922 | 1, 2, 3, 4 |
| 341 | 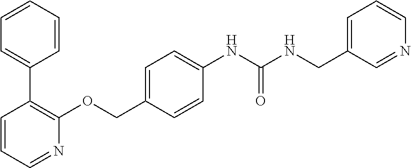 [SMILES: O=C(NCc1cccnc1)Nc1ccc(COc2ncccc2-c2ccccc2)cc1] | 1-(4-{[(3- Phenylpyridin-2- yl)oxy]methyl} phenyl)-3-(pyridin-3- ylmethyl)urea | 411.1815 | 7, 8, 4 |
| 342 | 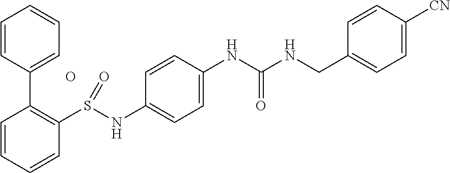 | N-(4-{[(4- cyanobenzyl) carbamoyl]amino} phenyl) biphenyl-2- sulfonamide | 483.1482 | 17, 18 |
| 343 | 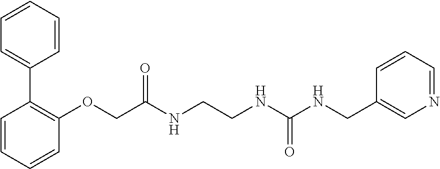 [SMILES: O=C(COc1ccccc1-c1ccccc1)NCCNC(=O)NCc1cccnc1] | 2-(Biphenyl-2- yloxy)-N-(2- {[(pyridin-3- ylmethyl)carbamoyl] amino}ethyl)acetamide | 405.1964 | 10, 15, 40, 18 |
| 344 | 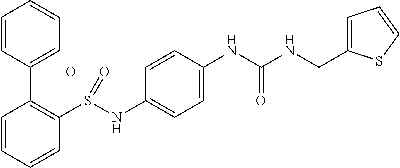 | N-(4-{[(thiophen-2- ylmethyl)carbamoyl] aminolphenyl) biphenyl- 2-sulfonamide | 464.0942 | 17, 18 |
| 345 | 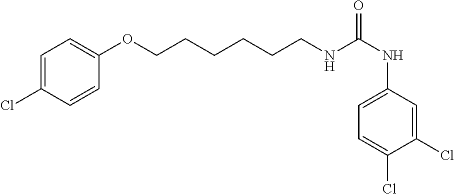 [SMILES: O=C(NCCCCCCOc1ccc(Cl)cc1)Nc1ccc(Cl)c(Cl)c1] | 1-[6-(4- Chlorophenoxy) hexyl]-3-(3,4- dichlorophenyl)urea | 415.0736 | 41, 42, 47 |
| 346 | 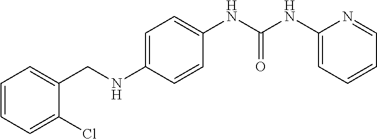 [SMILES: O=C(Nc1ccc(NCc2ccccc2Cl)cc1)Nc1ccccn1] | 1-{4-[(2- Chlorobenzyl)amino] phenyl}-3-pyridin-2- ylurea | 353.1228 | 1, 2, 35 |
| 347 | 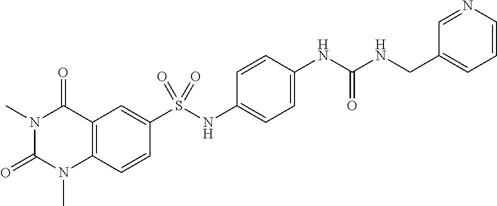 [SMILES: Cn1c(=O)c2cc(S(=O)(=O)Nc3ccc(NC(=O)NCc4cccnc4)cc3)ccc2n(C)c1=O] | 1,3-dimethyl-2,4- dioxo-N-(4- {[(pyridin-3- ylmethyl)carbamoyl] amino}phenyl)- 1,2,3,4- tetrahydroquinazoline- 6-sulfonamide | 495.1455 | 1, 2, 3 |
| 348 | 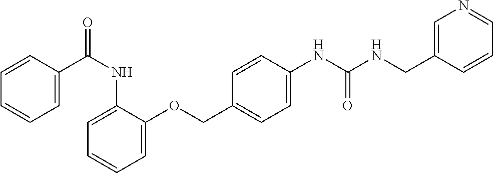 [SMILES: O=C(NCc1cccnc1)Nc1ccc(COc2ccccc2NC(=O)c2ccccc2)cc1] | N-{2-[(4-{[(Pyridin- 3- ylmethyl)carbamoyl] amino}benzyl)oxy] phenyl}benzamide | 453.1972 | 65, 7, 8, 32 |
| 349 | 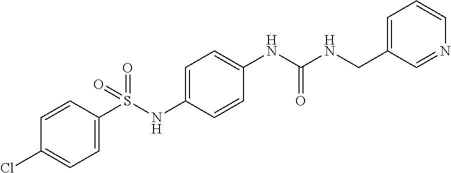 [SMILES: O=C(NCc1cccnc1)Nc1ccc(NS(=O)(=O)c2ccc(Cl)cc2)cc1] | 4-Chloro-N-(4- {[(pyridin-3- ylmethyl)carbamoyl] amino}phenyl) benzenesulfonamide | 417.0893 | 1, 2, 3 |
| 350 | 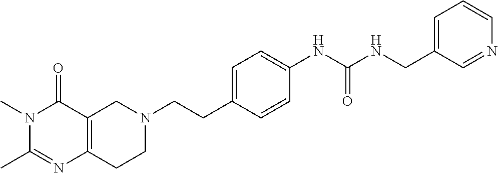 [SMILES: Cc1nc2c(c(=O)n1C)CN(CCc1ccc(NC(=O)NCc3cccnc3)cc1)CC2] | 1-{4-[2-(2,3- Dimethyl-4-oxo- 3,5,7,8- tetrahydropyrido[4,3- d]pyrimidin-6(4H)- yl)ethyl]phenyl}-3- (pyridin-3- ylmethyl)urea | 433.2327 | 30, 31, 32, 37, 2, 18 |
| 351 | 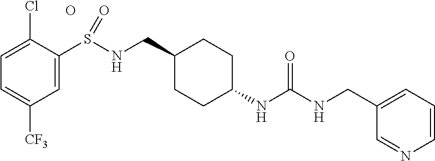 | 2-Chloro-N-[(trans- 4-{[(pyridin-3- ylmethyl)carbamoyl] amino}cyclohexyl) methyl]-5- (trifluoromethyl) benzenesulfonamide | 505.1298 | 3, 32, 46 |
| 352 | 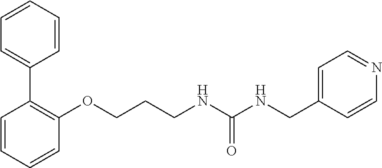 [SMILES: O=C(NCCCOc1ccccc1-c1ccccc1)NCc1ccncc1] | 1-[3-(Biphenyl-2- yloxy)propyl]-3- (pyridin-4- ylmethyl)urea | 384.17355 [M + Na]+ | 10, 18 |
| 353 | 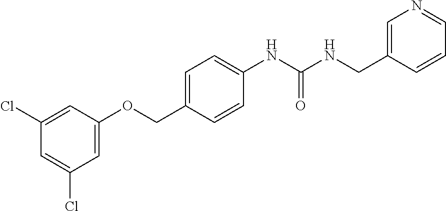 [SMILES: O=C(NCc1cccnc1)Nc1ccc(COc2cc(Cl)cc(Cl)c2)cc1] | 1-{4-[(3,5- Dichlorophenoxy) methyl]phenyl}-3- (pyridin-3- ylmethyl)urea | 402.0769 | 7, 8 |
| 354 | 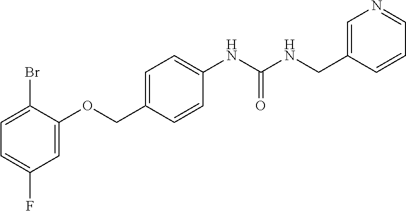 [SMILES: O=C(NCc1cccnc1)Nc1ccc(COc2cc(F)ccc2Br)cc1] | 1-{4-[(2-Bromo-5- fluorophenoxy)methyl] phenyl}-3-(pyridin- 3-ylmethyl)urea | 430.0556 | 7, 8 |
| 355 | 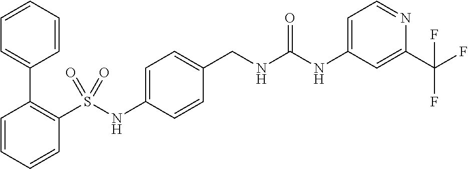 [SMILES: O=C(NCc1ccc(NS(=O)(=O)c2ccccc2-c2ccccc2)cc1)Nc1ccnc(C(F)(F)F)c1] | N-{4-[({[2- (Trifluoromethyl) pyridin-4- yl]carbamoyl}amino) methyl]phenyl} biphenyl- 2-sulfonamide | 527.1352 | 17, 18 |
| 356 | 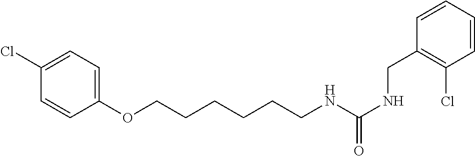 [SMILES: O=C(NCCCCCCOc1ccc(Cl)cc1)NCc1ccccc1Cl] | 1-(2-Chlorobenzyl)- 3-[6-(4- chlorophenoxy)hexyl] urea | 395.1279 | 41, 42, 47 |
| 357 | 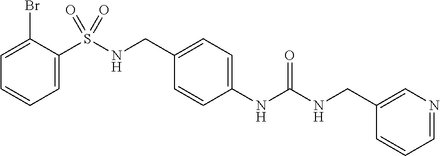 [SMILES: O=C(NCc1cccnc1)Nc1ccc(CNS(=O)(=O)c2ccccc2Br)cc1] | 2-Bromo-N-(4- {[(pyridin-3- ylmethyl)carbamoyl] amino}benzyl) benzenesulfonamide | 475.0453 | 18, 32 |
| 358 | 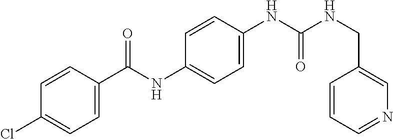 [SMILES: O=C(NCc1cccnc1)Nc1ccc(NC(=O)c2ccc(Cl)cc2)cc1] | 4-Chloro-N-(4- {[(pyridin-3- ylmethyl)carbamoyl] amino}phenyl) benzamide | 381.1116 | 1, 2, 9 |
| 359 | 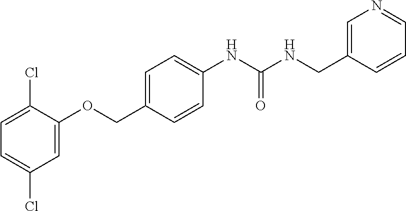 [SMILES: O=C(NCc1cccnc1)Nc1ccc(COc2cc(Cl)ccc2Cl)cc1] | 1-{4-[(2,5- Dichlorophenoxy) methyl]phenyl}-3- (pyridin-3- ylmethyl)urea | 402.0784 | 7, 8 |
| 360 | 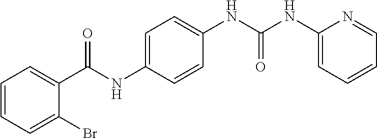 [SMILES: O=C(Nc1ccc(NC(=O)c2ccccc2Br)cc1)Nc1ccccn1] | 2-Bromo-N-{4- [(pyridin-2- ylcarbamoyl)amino] phenyl}benzamide | 411.0455 | 1, 2, 40 |
| 361 | 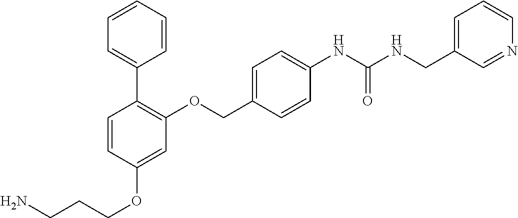 [SMILES: NCCCOc1ccc(-c2ccccc2)c(OCc2ccc(NC(=O)NCc3cccnc3)cc2)c1] | 1-[4-({[4-(3- Aminopropoxy) biphenyl-2- yl]oxy}methyl)phenyl]- 3-(pyridin-3- ylmethyl)urea | 483.2400 | 70, 44, 4, 20, 46, 10, 32 |
| 362 | 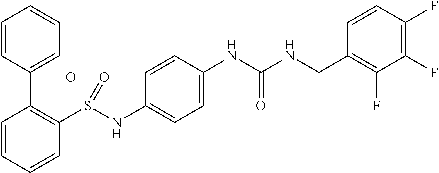 | N-(4-{[(2,3,4- trifluorobenzyl) carbamoyl]amino} phenyl) biphenyl-2- sulfonamide | 512.1252 | 17, 18 |
| 363 | 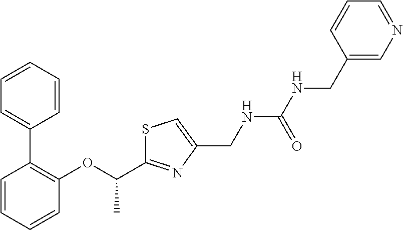 [SMILES: C[C@H](Oc1ccccc1-c1ccccc1)c1nc(CNC(=O)NCc2cccnc2)cs1] | 1-({2-[(1S)-1- (Biphenyl-2- yloxy)ethyl]-1,3- thiazol-4-yl}methyl)- 3-(pyridin-3- ylmethyl)urea | 445.1690 | 14, 15, 24, 25, 3, 18 |
| 364 | 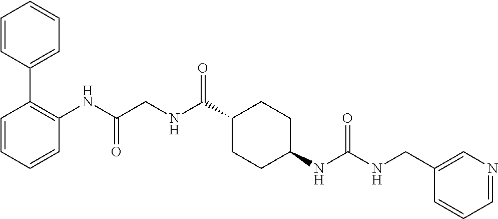 [SMILES: O=C(CNC(=O)[C@H]1CC[C@H](NC(=O)NCc2cccnc2)CC1)Nc1ccccc1-c1ccccc1] | trans-N-[2- (Biphenyl-2- ylamino)-2- oxoethyl]-4- {[(pyridin-3- ylmethyl)carbamoyl] amino} cyclohexanecarboxamide | 486.2488 | 40, 32, 40, 32, 18 |
| 365 | 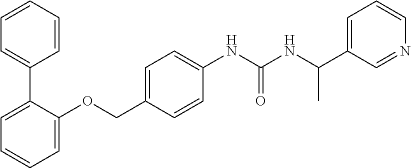 [SMILES: CC(NC(=O)Nc1ccc(COc2ccccc2-c2ccccc2)cc1)c1cccnc1] | 1-{4-[(Biphenyl-2- yloxy)methyl]phenyl}- 3-[1-(pyridin-3- yl)ethyl]urea | 424.2045 | 37, 32, 18 |
| 366 | 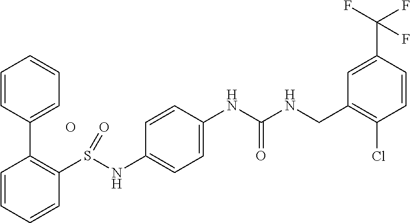 | N-[4-({[2-chloro-5- (trifluoromethyl) benzyl]carbamoyl} amino) phenyl]biphenyl-2- sulfonamide | 560.1000 | 17, 18 |
| 367 | 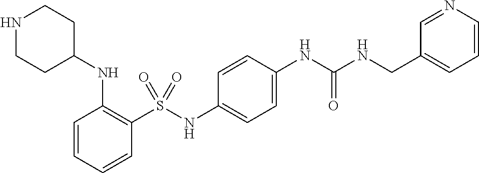 [SMILES: O=C(NCc1cccnc1)Nc1ccc(NS(=O)(=O)c2ccccc2NC2CCNCC2)cc1] | 2-(piperidin-4- ylamino)-N-(4- {[(pyridin-3- ylmethyl)carbamoyl] amino}phenyl) benzenesulfonamide | 481.9970 | 1, 2, 3, 22 |
| 368 | 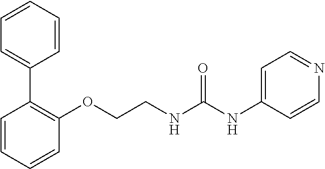 [SMILES: O=C(NCCOc1ccccc1-c1ccccc1)Nc1ccncc1] | 1-[2-(Biphenyl-2- yloxy)ethyl]-3- pyridin-4-ylurea | 334.1586 | 10, 18 |
| 369 | 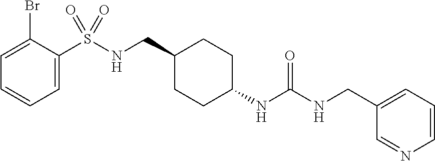 [SMILES: O=C(NCc1cccnc1)N[C@H]1CC[C@H](CNS(=O)(=O)c2ccccc2Br)CC1] | 2-Bromo-N-[(trans- 4-{[(pyridin-3- ylmethyl)carbamoyl] amino}cyclohexyl) methyl] benzenesulfonamide | 481.0904 | 72, 32, 46, 66 |
| 370 | 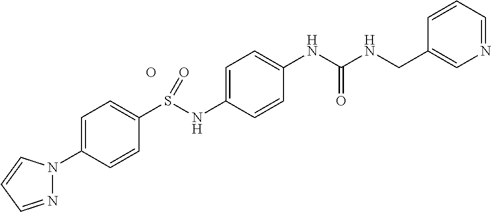 | 4-(1H-Pyrazol-1-yl)- N-(4-{[(pyridin-3- ylmethyl)carbamoyl] amino}phenyl) benzenesulfonamide | 449.1400 | 18, 3 |
| 371 | 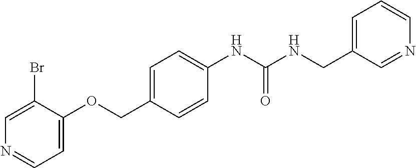 [SMILES: O=C(NCc1cccnc1)Nc1ccc(COc2ccncc2Br)cc1] | 1-(4-{[(3- Bromopyridin-4- yl)oxy]methyl}phenyl)- 3-(pyridin-3- ylmethyl)urea | 413.0665 | 7, 8, 4 |
| 372 | 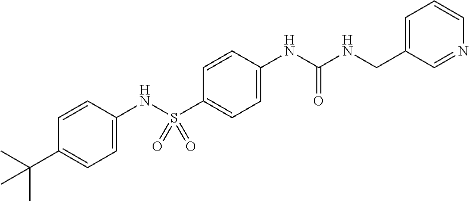 [SMILES: CC(C)(C)c1ccc(NS(=O)(=O)c2ccc(NC(=O)NCc3cccnc3)cc2)cc1] | N-(4-tert- Butylphenyl)-4- {[(pyridin-3- ylmethyl)carbamoyl] amino} benzenesulfonamide | 439.1809 | 5, 2, 18 |
| 373 | 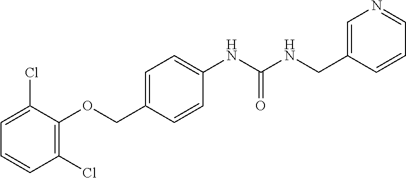 [SMILES: O=C(NCc1cccnc1)Nc1ccc(COc2c(Cl)cccc2Cl)cc1] | 1-{4-[(2,6- Dichlorophenoxy) methyl]phenyl}-3- (pyridin-3- ylmethyl)urea | 402.0787 | 7, 8 |
| 374 | 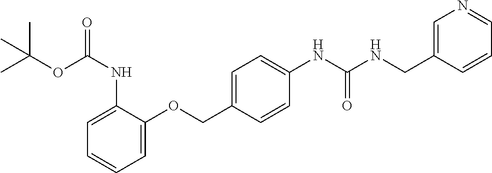 [SMILES: CC(C)(C)OC(=O)Nc1ccccc1OCc1ccc(NC(=O)NCc2cccnc2)cc1] | tert-Butyl {2-[(4- {[(pyridin-3- ylmethyl)carbamoyl] amino}benzyl)oxy] phenyl}carbamate | NA | 46, 45, 44 |
| 375 | 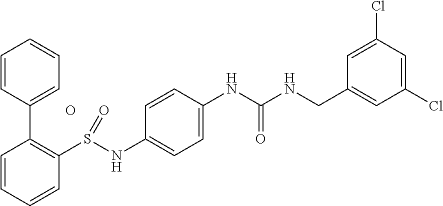 | N-(4-{[(3,5- dichlorobenzyl) carbamoyl]amino} phenyl) biphenyl-2- sulfonamide | 526.0734 | 17, 18 |
| 376 | 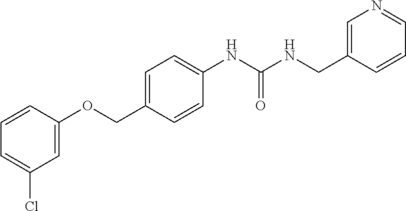 [SMILES: O=C(NCc1cccnc1)Nc1ccc(COc2cccc(Cl)c2)cc1] | 1-{4-[(3- Chlorophenoxy) methyl]phenyl}-3- (pyridin-3- ylmethyl)urea | 368.1160 | 7, 8 |
| 377 | 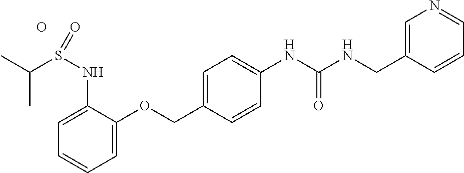 | N-{2-[(4-{[(Pyridin- 3- ylmethyl)carbamoyl] amino}benzyl)oxy] phenyl}propane-2- sulfonamide | 455.1731 | 65, 7, 8, 32 |
| 378 | 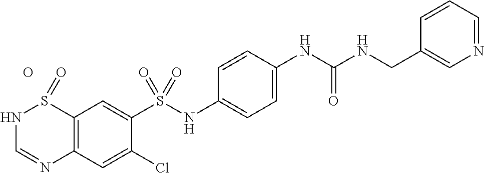 | 6-chloro-N-(4- {[(pyridin-3- ylmethyl)carbamoyl] amino}phenyl)-2H- 1,2,4- benzothiadiazine-7- sulfonamide 1,1- dioxide | 521.0368 | 28, 29, 1, 2, 3 |
| 379 | 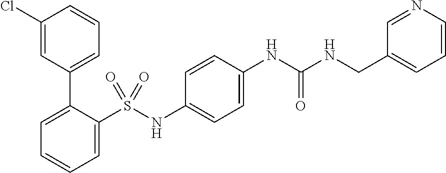 [SMILES: O=C(NCc1cccnc1)Nc1ccc(NS(=O)(=O)c2ccccc2-c2cccc(Cl)c2)cc1] | 3′-chloro-N-(4- {[(pyridin-3- ylmethyl)carbamoyl] amino}phenyl)biphenyl- 2-sulfonamide | 493.1101 | 1, 2, 3, 4 |
| 380 | 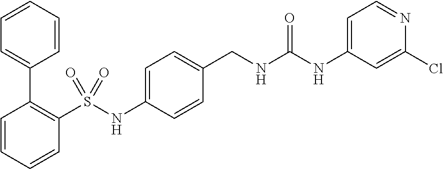 [SMILES: O=C(NCc1ccc(NS(=O)(=O)c2ccccc2-c2ccccc2)cc1)Nc1ccnc(Cl)c1] | N-[4-({[(2- Chloropyridin-4- yl)carbamoyl]amino} methyl)phenyl] biphenyl-2-sulfonamide | 493.1099 | 17, 18 |
| 381 | 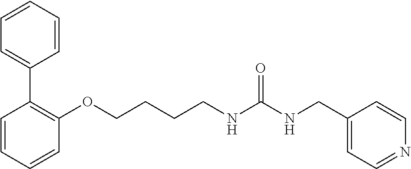 [SMILES: O=C(NCCCCOc1ccccc1-c1ccccc1)NCc1ccncc1] | 1-[4-(Biphenyl-2- yloxy)butyl]-3- (pyridin-4- ylmethyl)urea | 398.18924 [M + Na]+ | 10, 18 |
| 382 | 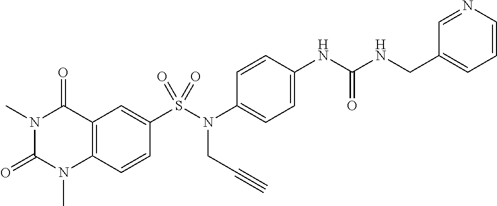 [SMILES: C#CCN(c1ccc(NC(=O)NCc2cccnc2)cc1)S(=O)(=O)c1ccc2c(c1)c(=O)n(C)c(=O)n2C] | 1,3-dimethyl-2,4- dioxo-N-(prop-2-yn- 1-yl)-N-(4- {[(pyridin-3- ylmethyl)carbamoyl] amino}phenyl)- 1,2,3,4- tetrahydroquinazoline- 6-sulfonamide | 533.1610 | 1, 2, 3, 19 |
| 383 | 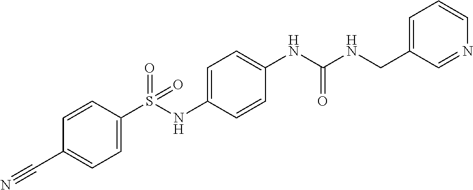 [SMILES: N#Cc1ccc(S(=O)(=O)Nc2ccc(NC(=O)NCc3cccnc3)cc2)cc1] | 4-Cyano-N-(4- {[(pyridin-3- ylmethyl)carbamoyl] amino}phenyl) benzenesulfonamide | 408.1114 | 1, 2, 3 |
| 384 | 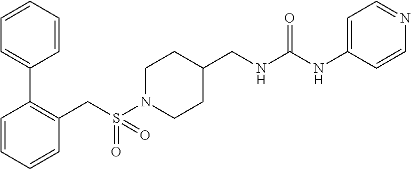 [SMILES: O=C(NCC1CCN(S(=O)(=O)Cc2ccccc2-c2ccccc2)CC1)Nc1ccncc1] | 1-({1-[(Biphenyl-2- ylmethyl)sulfonyl] piperidin-4-yl}methyl)- 3-pyridin-4-ylurea | 465.2044 | 18, 32, 3, 4 |
| 385 | 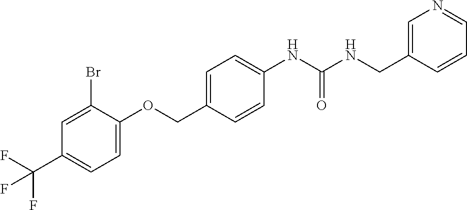 [SMILES: O=C(NCc1cccnc1)Nc1ccc(COc2ccc(C(F)(F)F)cc2Br)cc1] | 1-(4-{[2-Bromo-4- (trifluoromethyl) phenoxy]methyl} phenyl)- 3-(pyridin-3- ylmethyl)urea | 480.0525 | 7, 8 |
| 386 | 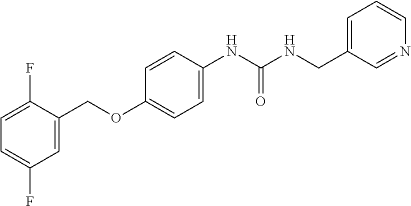 [SMILES: O=C(NCc1cccnc1)Nc1ccc(OCc2cc(F)ccc2F)cc1] | 1-{4-[(2,5- Difluorobenzyl)oxy] phenyl}-3-(pyridin- 3-ylmethyl)urea | 370.1391 | 44, 32, 18 |
| 387 | 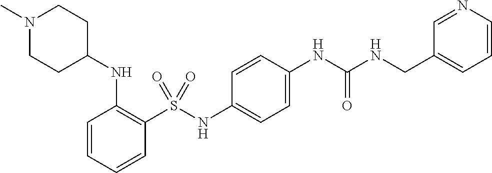 [SMILES: CN1CCC(Nc2ccccc2S(=O)(=O)Nc2ccc(NC(=O)NCc3cccnc3)cc2)CC1] | 2-[(1- methylpiperidin-4- yl)amino]-N-(4- {[(pyridin-3- ylmethyl)carbamoyl] amino}phenyl) benzenesulfonamide | 495.2154 | 1, 2, 3, 22 |
| 388 | 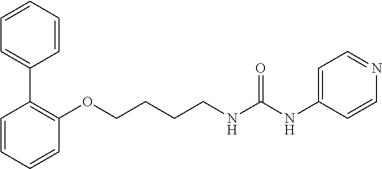 [SMILES: O=C(NCCCCOc1ccccc1-c1ccccc1)Nc1ccncc1] | 1-[4-(biphenyl-2- yloxy)butyl]-3- pyridin-4-ylurea | 462.1877 | 10, 18 |
| 389 | 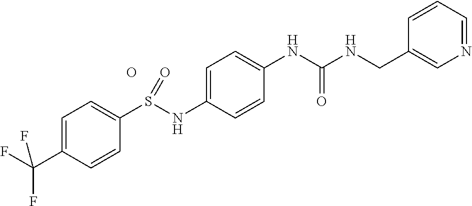 | N-(4-{[(Pyridin-3- ylmethyl)carbamoyl] amino}phenyl)-4- (trifluoromethyl) benzenesulfonamide | | 18, 3 |
| 390 | 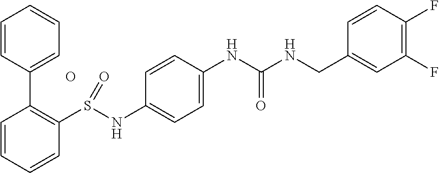 | N-(4-{[(3,4- difluorobenzyl) carbamoyl]amino} phenyl) biphenyl-2- sulfonamide | 494.1341 | 17, 18 |
| 391 | 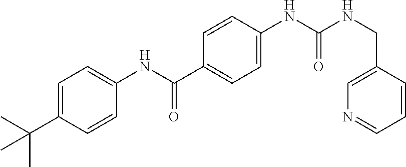 [SMILES: CC(C)(C)c1ccc(NC(=O)c2ccc(NC(=O)NCc3cccnc3)cc2)cc1] | N-(4-tert- butylphenyl)-4- {[(pyridin-3- ylmethyl)carbamoyl] amino}benzamide | 403.2191 | 1, 16, 9 |
| 392 | 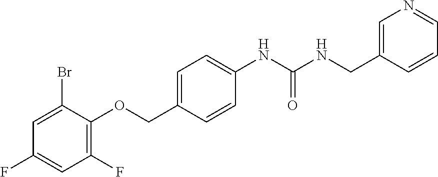 [SMILES: O=C(NCc1cccnc1)Nc1ccc(COc2c(F)cc(F)cc2Br)cc1] | 1-{4-[(2-Bromo-4,6- difluorophenoxy) methyl]phenyl}-3- (pyridin-3- ylmethyl)urea | 448.0430 | 7, 8 |
| 393 | 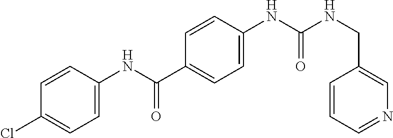 [SMILES: O=C(NCc1cccnc1)Nc1ccc(C(=O)Nc2ccc(Cl)cc2)cc1] | N-(4-Chlorophenyl)- 4-{[(pyridin-3- ylmethyl)carbamoyl] amino}benzamide | 381.1126 | 1, 15, 9 |
| 394 | 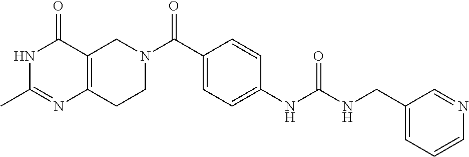 [SMILES: Cc1nc2c(c(=O)[nH]1)CN(C(=O)c1ccc(NC(=O)NCc3cccnc3)cc1)CC2] | 1-{4-[(2-Methyl-4- oxo-1,5,7,8- tetrahydropyrido[4,3- d]pyrimidin-6(4H)- yl)carbonyl]phenyl}- 3-(pyridin-3- ylmethyl)urea | 419.1771 | 30, 32, 33, 34 |
| 395 | 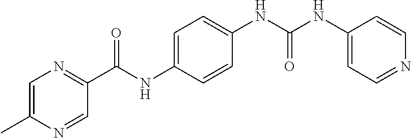 [SMILES: Cc1cnc(C(=O)Nc2ccc(NC(=O)Nc3ccncc3)cc2)cn1] | 5-Methyl-N-{4- [(pyridin-4- ylcarbamoyl)amino] phenyl}pyrazine-2- carboxamide | 349.1468 | 1, 2, 40 |
| 396 | 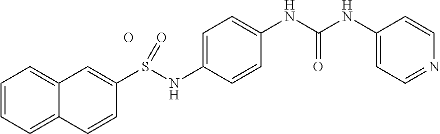 | N-{4-[(Pyridin-4- ylcarbamoyl)amino] phenyl}naphthalene-2- sulfonamide | 419.1218 | 1, 2, 3 |
| 397 | 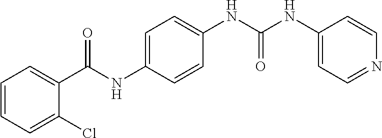 [SMILES: O=C(Nc1ccncc1)Nc1ccc(NC(=O)c2ccccc2Cl)cc1] | 2-Chloro-N-{4- [(pyridin-4- ylcarbamoyl)amino] phenyl}benzamide | 367.1404 | 1, 2, 40 |
| 398 | 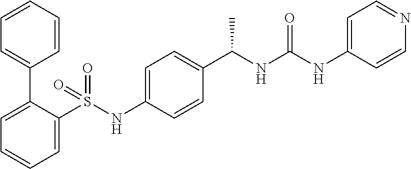 [SMILES: C[C@H](NC(=O)Nc1ccncc1)c1ccc(NS(=O)(=O)c2ccccc2-c2ccccc2)cc1] | N-(4-{(1S)-1- [(Pyridin-4- ylcarbamoyl)amino] ethyl}phenyl)biphenyl- 2-sulfonamide | 473.1681 | 17, 18 |
| 399 | 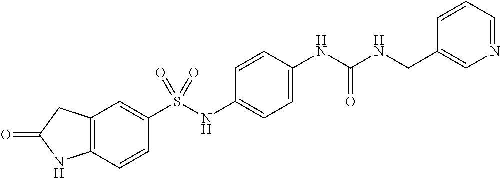 [SMILES: O=C1Cc2cc(S(=O)(=O)Nc3ccc(NC(=O)NCc4cccnc4)cc3)ccc2N1] | 2-oxo-N-(4- {[(pyridin-3- ylmethyl)carbamoyl] amino}phenyl)-2,3- dihydro-1H-indole-5- sulfonamide | 438.1227 | 18, 3 |
| 400 | 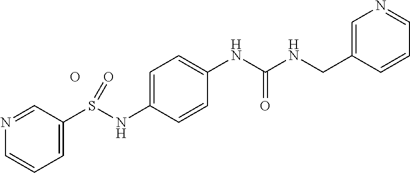 | N-(4-{[(pyridin-3- ylmethyl)carbamoyl] amino}phenyl)pyridine- 3-sulfonamide | 384.1128 | 1, 2, 3 |
| 401 | 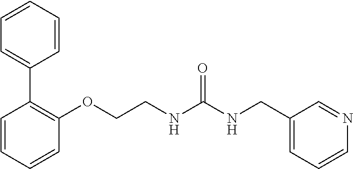 [SMILES: O=C(NCCOc1ccccc1-c1ccccc1)NCc1cccnc1] | 1-[2-(Biphenyl-2- yloxy)ethyl]-3- (pyridin-3- ylmethyl)urea | 348.1734 | 10, 18 |
| 402 | 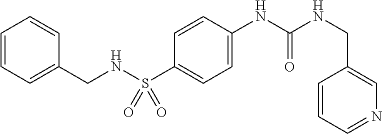 [SMILES: O=C(NCc1cccnc1)Nc1ccc(S(=O)(=O)NCc2ccccc2)cc1] | N-Benzyl-4- {[(pyridin-3- ylmethyl)carbamoyl] amino} benzenesulfonamide | 397.1347 | 5, 2, 18 |
| 403 | 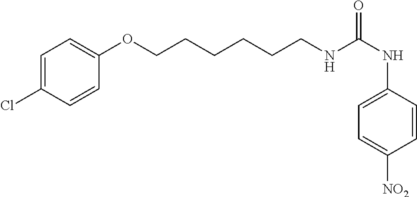 [SMILES: O=C(NCCCCCCOc1ccc(Cl)cc1)Nc1ccc([N+](=O)[O-])cc1] | 1-[6-(4- Chlorophenoxy)hexyl]- 3-(4- nitrophenyl)urea | 392.1369 | 41, 42, 47 |
| 404 | 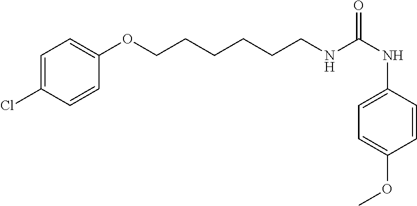 [SMILES: COc1ccc(NC(=O)NCCCCCCOc2ccc(Cl)cc2)cc1] | 1-[6-(4- Chlorophenoxy)hexyl]- 3-(4- methoxyphenyl)urea | 377.1624 | 41, 42, 47 |
| 405 | 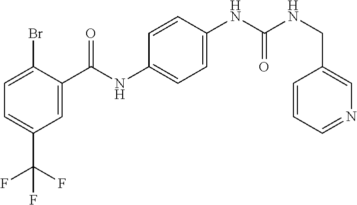 [SMILES: O=C(NCc1cccnc1)Nc1ccc(NC(=O)c2cc(C(F)(F)F)ccc2Br)cc1] | 2-Bromo-N-(4- {[(pyridin-3- ylmethyl)carbamoyl] amino}phenyl)-5- (trifluoromethyl) benzamide | 493.0482 | 1, 2, 9 |
| 406 | 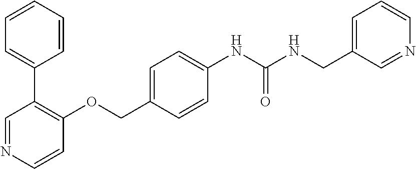 [SMILES: O=C(NCc1cccnc1)Nc1ccc(COc2ccncc2-c2ccccc2)cc1] | 1-(4-{[(3- Phenylpyridin-4- yl)oxy]methyl} phenyl)- 3-(pyridin-3- ylmethyl)urea | 411.1816 | 7, 8, 4 |
| 407 | 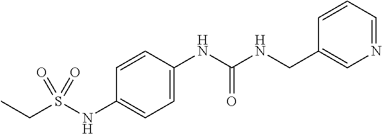 [SMILES: CCS(=O)(=O)Nc1ccc(NC(=O)NCc2cccnc2)cc1] | N-(4-{[(pyridin-3- ylmethyl)carbamoyl] amino}phenyl) ethanesulfonamide | 335.1170 | 18, 3 |
| 408 | 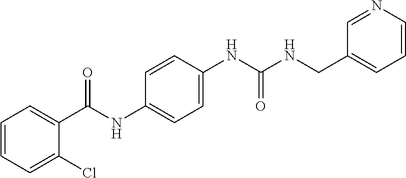 [SMILES: O=C(NCc1cccnc1)Nc1ccc(NC(=O)c2ccccc2Cl)cc1] | 2-Chloro-N-(4- {[(pyridin-3- ylmethyl)carbamoyl] amino}phenyl) benzamide | 381.1103 | 1, 2, 9 |
| 409 | 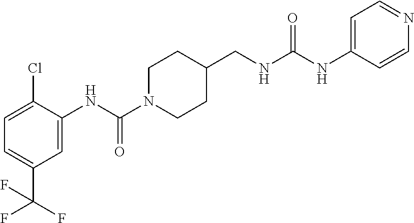 [SMILES: O=C(NCC1CCN(C(=O)Nc2cc(C(F)(F)F)ccc2Cl)CC1)Nc1ccncc1] | N-[2-Chloro-5- (trifluoromethyl) phenyl]-4-{[(pyridin-4- ylcarbamoyl)amino] methyl}piperidine-1- carboxamide | 456.1373 | 18, 32, 18 |
| 410 | 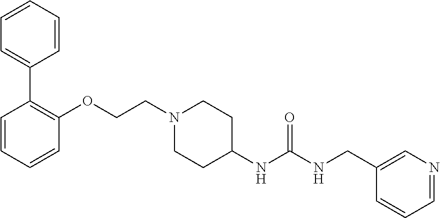 [SMILES: O=C(NCc1cccnc1)NC1CCN(CCOc2ccccc2-c2ccccc2)CC1] | 1-{1-[2-(Biphenyl-2- yloxy)ethyl]piperidin- 4-yl}-3-(pyridin-3- ylmethyl)urea | 431.2431 | 18, 32, 37 |
| 411 | 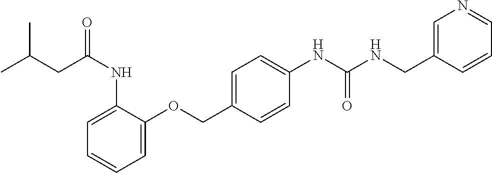 [SMILES: CC(C)CC(=O)Nc1ccccc1OCc1ccc(NC(=O)NCc2cccnc2)cc1] | 3-Methyl-N-{2-[(4- {[(pyridin-3- ylmethyl)carbamoyl] amino}benzyl)oxy] phenyl}butanamide | 433.2233 | 65, 7, 8, 32 |
| 412 | 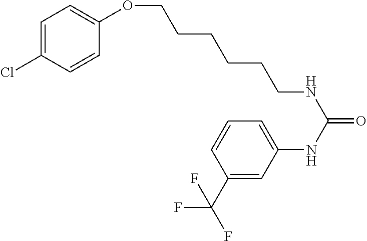 [SMILES: O=C(NCCCCCCOc1ccc(Cl)cc1)Nc1cccc(C(F)(F)F)c1] | 1-[6-(4- Chlorophenoxy)hexyl]- 3-[3- (trifluoromethyl) phenyl]urea | 415.1396 | 41, 42, 47 |
| 413 | 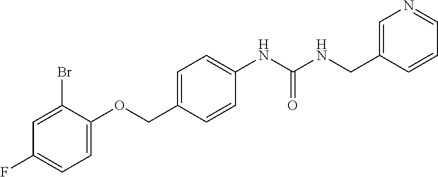 [SMILES: O=C(NCc1cccnc1)Nc1ccc(COc2ccc(F)cc2Br)cc1] | 1-{4-[(2-Bromo-4- fluorophenoxy)methyl] phenyl}-3-(pyridin- 3-ylmethyl)urea | 430.0551 | 7, 8 |
| 414 | 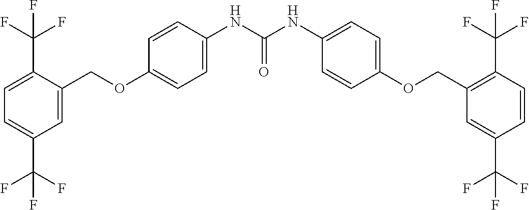 [SMILES: O=C(Nc1ccc(OCc2cc(C(F)(F)F)ccc2C(F)(F)F)cc1)Nc1ccc(OCc2cc(C(F)(F)F)ccc2C(F)(F)F)cc1] | 1,3-Bis(4-{[2,5- bis(trifluoromethyl) benzyl]oxy}phenyl)urea | 697.1365 | 44, 32, 18 |
| 415 | 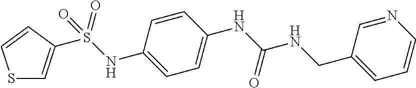 [SMILES: O=C(NCc1cccnc1)Nc1ccc(NS(=O)(=O)c2ccsc2)cc1] | N-(4-{[(Pyridin-3- ylmethyl)carbamoyl] amino}phenyl)thiophene- 3-sulfonamide | 389.0781 | 1, 2, 3 |
| 416 | 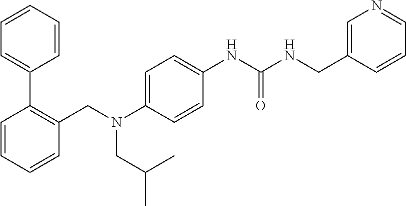 [SMILES: CC(C)CN(Cc1ccccc1-c1ccccc1)c1ccc(NC(=O)NCc2cccnc2)cc1] | 1-{4-[(Biphenyl-2- ylmethyl)(2- methylpropyl)amino] phenyl}-3-(pyridin- 3-ylmethyl)urea | 435.2600 | 1, 2, 35, 35 |
| 417 | 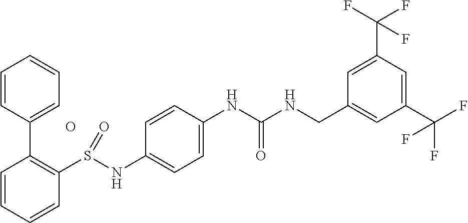 | N-[4-({[3,5- bis(trifluoromethyl) benzyl]carbamoyl} amino)phenyl]biphenyl- 2-sulfonamide | 594.1285 | 17, 18 |
| 418 | 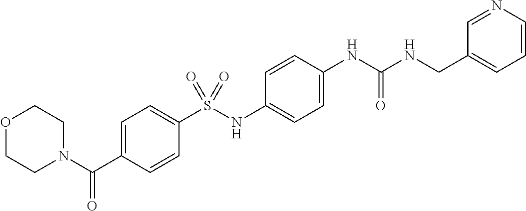 [SMILES: O=C(NCc1cccnc1)Nc1ccc(NS(=O)(=O)c2ccc(C(=O)N3CCOCC3)cc2)cc1] | 4-(Morpholin-4- ylcarbonyl)-N-(4- {[(pyridin-3- ylmethyl)carbamoyl] amino}phenyl) benzenesulfonamide | 496.1655 | 1, 2, 3, 9 |
| 419 | 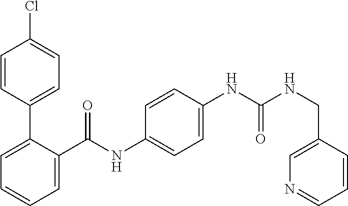 [SMILES: O=C(NCc1cccnc1)Nc1ccc(NC(=O)c2ccccc2-c2ccc(Cl)cc2)cc1] | 4′-chloro-N-(4- {[(pyridin-3- ylmethyl)carbamoyl] amino}phenyl) biphenyl-2-carboxamide | 457.1433 | 1, 2, 3, 9 |
| 420 | 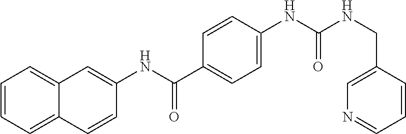 [SMILES: O=C(NCc1cccnc1)Nc1ccc(C(=O)Nc2ccc3ccccc3c2)cc1] | N-(naphthalen-2-yl)- 4-{[(pyridin-3- ylmethyl)carbamoyl] amino}benzamide | 397.1683 | 1, 16, 9 |
| 421 | 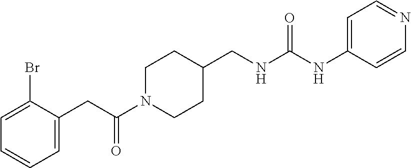 [SMILES: O=C(NCC1CCN(C(=O)Cc2ccccc2Br)CC1)Nc1ccncc1] | 1-({1-[(2- Bromophenyl)acetyl] piperidin-4- yl}methyl)-3- pyridin-4-ylurea | 433.1066 | 18, 32, 9 |
| 422 | 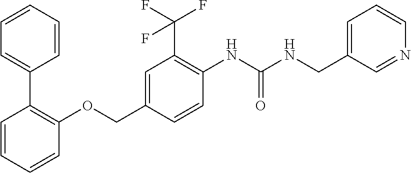 [SMILES: O=C(NCc1cccnc1)Nc1ccc(COc2ccccc2-c2ccccc2)cc1C(F)(F)F] | 1-{4-[(Biphenyl-2- yloxy)methyl]-2- (trifluoromethyl) phenyl}-3-(pyridin-3- ylmethyl)urea | 478.1748 | 37, 32, 18 |
| 423 | 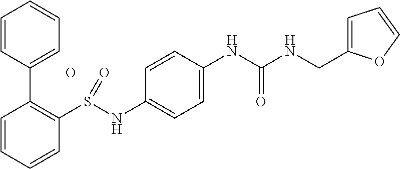 | N-(4-{[(furan-2- ylmethyl)carbamoyl] amino}phenyl)biphenyl- 2-sulfonamide | 448.1322 | 17, 18 |
| 424 | 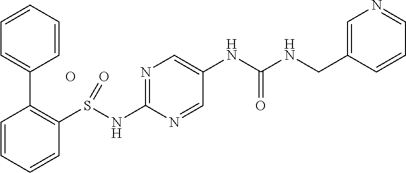 | N-(5-{[(Pyridin-3- ylmethyl)carbamoyl] amino}pyrimidin-2- yl)biphenyl-2- sulfonamide | 461.1374 | 18, 16, 3 |
| 425 | 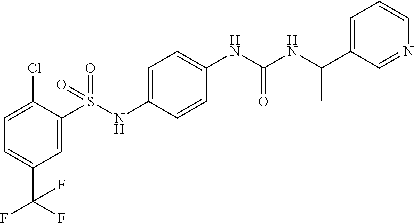 [SMILES: CC(NC(=O)Nc1ccc(NS(=O)(=O)c2cc(C(F)(F)F)ccc2Cl)cc1)c1cccnc1] | 2-chloro-N-[4-({[1- (pyridin-3- yl)ethyl]carbamoyl} amino)phenyl]-5- (trifluoromethyl) benzenesulfonamide | 499.0738 | 17, 18 |
| 426 | 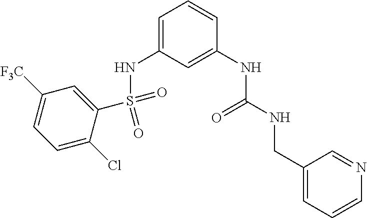 [SMILES: O=C(NCc1cccnc1)Nc1cccc(NS(=O)(=O)c2cc(C(F)(F)F)ccc2Cl)c1] | 2-Chloro-N-(3- {[(pyridin-3- ylmethyl)carbamoyl] amino}phenyl)-5- (trifluoromethyl) benzenesulfonamide | 485.0639 | 1, 2, 3 |
| 427 | 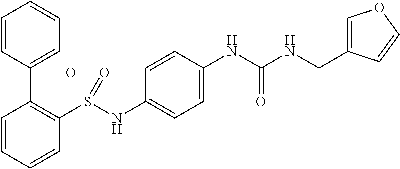 | N-(4-{[(furan-3- ylmethyl)carbamoyl] amino}phenyl)biphenyl- 2-sulfonamide | 448.1318 | 17, 18 |
| 428 | 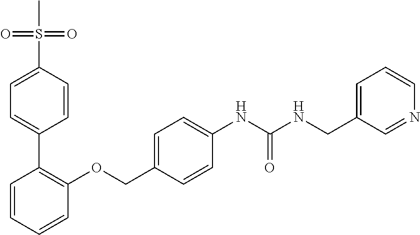 [SMILES: CS(=O)(=O)c1ccc(-c2ccccc2OCc2ccc(NC(=O)NCc3cccnc3)cc2)cc1] | 1-[4-({[4′- (Methylsulfonyl) biphenyl-2- yl]oxy}methyl)phenyl]- 3-(pyridin-3- ylmethyl)urea | 488.1620 | 44, 45, 46, 4 |
| 429 | 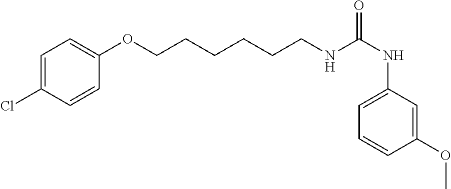 [SMILES: COc1cccc(NC(=O)NCCCCCCOc2ccc(Cl)cc2)c1] | 1-[6-(4- Chlorophenoxy)hexyl]- 3-(3- methoxyphenyl)urea | 377.1622 | 41, 42, 47 |
| 430 | 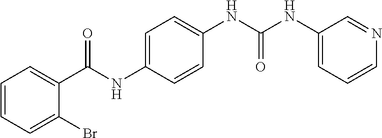 [SMILES: O=C(Nc1ccc(NC(=O)c2ccccc2Br)cc1)Nc1cccnc1] | 2-Bromo-N-{4- [(pyridin-3- ylcarbamoyl)amino] phenyl}benzamide | 411.0459 | 1, 2, 40 |
| 431 | 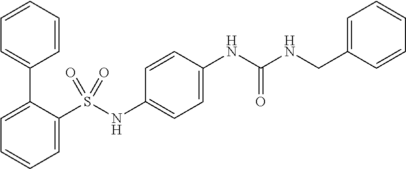 [SMILES: O=C(NCc1ccccc1)Nc1ccc(NS(=O)(=O)c2ccccc2-c2ccccc2)cc1] | N-{4- [(Benzylcarbamoyl) amino]phenyl}biphenyl- 2-sulfonamide | 458.1539 | 1, 2, 3 |
| 432 | 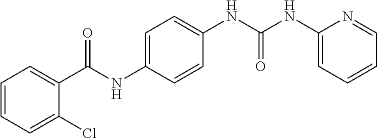 [SMILES: O=C(Nc1ccc(NC(=O)c2ccccc2Cl)cc1)Nc1ccccn1] | 2-Chloro-N-{4- [(pyridin-2- ylcarbamoyl)amino] phenyl}benzamide | 367.0957 | 1, 2, 40 |
| 433 | 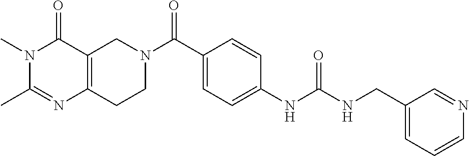 [SMILES: Cc1nc2c(c(=O)n1C)CN(C(=O)c1ccc(NC(=O)NCc3cccnc3)cc1)CC2] | 1-{4-[(2,3-dimethyl- 4-oxo-3,5,7,8- tetrahydropyrido[4,3- d]pyrimidin-6(4H)- yl)carbonyl]phenyl}- 3-(pyridin-3- ylmethyl)urea | 433.1878 | 30, 31, 32, 33, 34 |
| 434 | 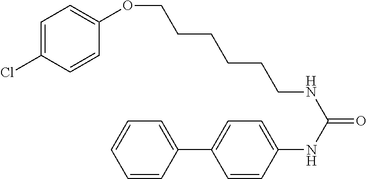 [SMILES: O=C(NCCCCCCOc1ccc(Cl)cc1)Nc1ccc(-c2ccccc2)cc1] | 1-Biphenyl-4-yl-3- [6-(4- chlorophenoxy)hexyl] urea | 423.1832 | 41, 42, 47 |
| 435 | 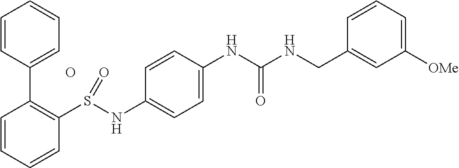 | N-(4-{[(3- methoxybenzyl) carbamoyl]amino} phenyl) biphenyl-2- sulfonamide | 488.1539 | 17, 18 |
| 436 | 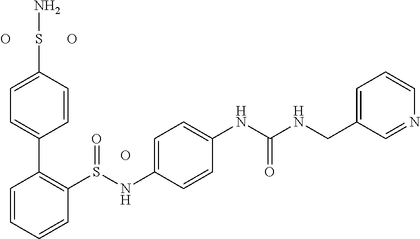 | N~2~-(4-{[(Pyridin- 3- ylmethyl)carbamoyl] amino}phenyl)biphenyl- 2,4′-disulfonamide | 538.1286 | 1, 2, 3, 4 |
| 437 | 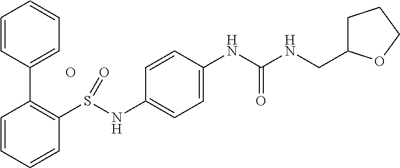 | N-(4- {[(tetrahydrofuran-2- ylmethyl)carbamoyl] amino}phenyl)biphenyl- 2-sulfonamide | 452.1635 | 17, 18 |
| 438 | 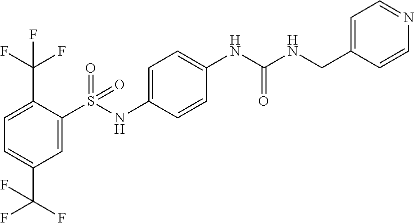 [SMILES: O=C(NCc1ccncc1)Nc1ccc(NS(=O)(=O)c2cc(C(F)(F)F)ccc2C(F)(F)F)cc1] | N-(4-{[(Pyridin-4- ylmethyl)carbamoyl] amino}phenyl)-2,5- bis(trifluoromethyl) benzenesulfonamide | 519.0910 | 1, 2, 3 |
| 439 | 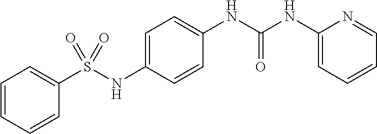 [SMILES: O=C(Nc1ccc(NS(=O)(=O)c2ccccc2)cc1)Nc1ccccn1] | N-{4-[(Pyridin-2- ylcarbamoyl)amino] phenyl} benzenesulfonamide | 369.1129 | 1, 2, 3 |
| 440 | 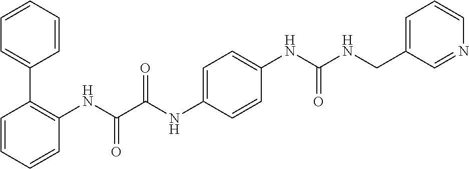 [SMILES: O=C(NCc1cccnc1)Nc1ccc(NC(=O)C(=O)Nc2ccccc2-c2ccccc2)cc1] | N-(Biphenyl-2-yl)- N′-(4-{[(pyridin-3- ylmethyl)carbamoyl] amino}phenyl) ethanediamide | 466.1909 | 81, 32, 18 |
| 441 | 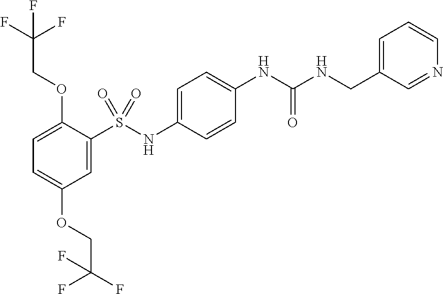 [SMILES: O=C(NCc1cccnc1)Nc1ccc(NS(=O)(=O)c2cc(OCC(F)(F)F)ccc2OCC(F)(F)F)cc1] | N-(4-{[(Pyridin-3- ylmethyl)carbamoyl] amino}phenyl)-2,5- bis(2,2,2- trifluoroethoxy) benzenesulfonamide | 579.1163 | 1, 2, 3 |
| 442 | 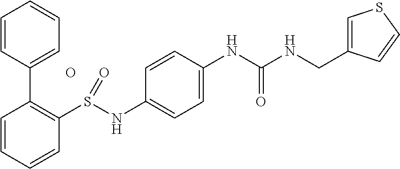 | N-(4-{[(thiophen-3- ylmethyl)carbamoyl] amino}phenyl)biphenyl- 2-sulfonamide | 464.1058 | 17, 18 |
| 443 | 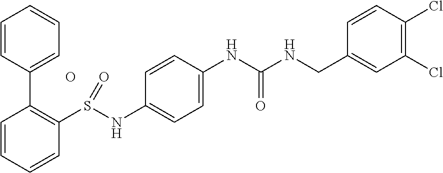 | N-(4-{[(3,4- dichlorobenzyl) carbamoyl]amino} phenyl) biphenyl-2- sulfonamide | 526.0752 | 17, 18 |
| 444 | 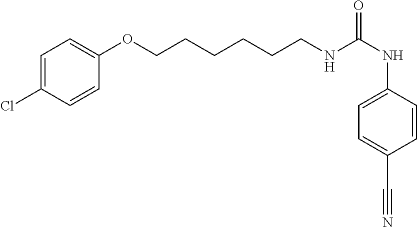 [SMILES: N#Cc1ccc(NC(=O)NCCCCCCOc2ccc(Cl)cc2)cc1] | 1-[6-(4- Chlorophenoxy)hexyl]- 3-(4- cyanophenyl)urea | 372.1464 | 41, 42, 47 |
| 445 | 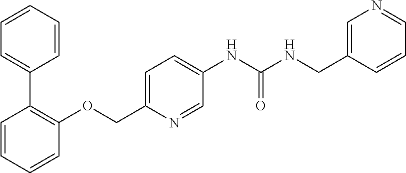 [SMILES: O=C(NCc1cccnc1)Nc1ccc(COc2ccccc2-c2ccccc2)nc1] | 1-{6-[(Biphenyl-2- yloxy)methyl]pyridin- 3-yl}-3-(pyridin-3- ylmethyl)urea | 411.1802 | 18, 10 |
| 446 | 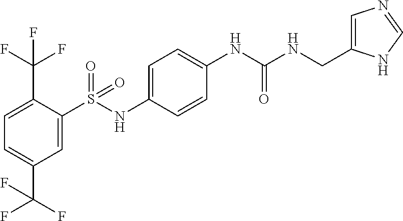 [SMILES: O=C(NCc1cnc[nH]1)Nc1ccc(NS(=O)(=O)c2cc(C(F)(F)F)ccc2C(F)(F)F)cc1] | N-(4-{[(1H- Imidazol-4- ylmethyl)carbamoyl] amino}phenyl)-2,5- bis(trifluoromethyl) benzenesulfonamide | 508.0817 | 3, 32, 18 |
| 447 | 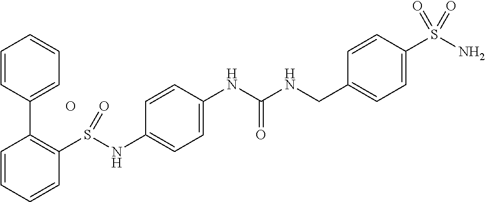 | N-(4-{[(4- sulfamoylbenzyl) carbamoyl]amino} phenyl) biphenyl-2- sulfonamide | 537.1261 | 17, 18 |
| 448 | 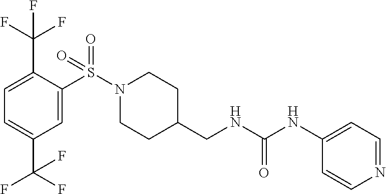 [SMILES: O=C(NCC1CCN(S(=O)(=O)c2cc(C(F)(F)F)ccc2C(F)(F)F)CC1)Nc1ccncc1] | 1-[(1-{[2,5- Bis(trifluoromethyl) phenyl]sulfonyl} piperidin-4-yl)methyl]-3- pyridin-4-ylurea | 511.1273 | 18, 32, 18 |
| 449 | 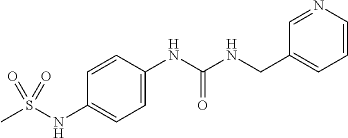 [SMILES: CS(=O)(=O)Nc1ccc(NC(=O)NCc2cccnc2)cc1] | N-(4-{[(pyridin-3- ylmethyl)carbamoyl] amino}phenyl) methanesulfonamide | 321.1023 | 1, 2, 3 |
| 450 | 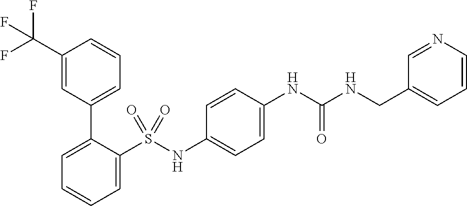 [SMILES: O=C(NCc1cccnc1)Nc1ccc(NS(=O)(=O)c2ccccc2-c2cccc(C(F)(F)F)c2)cc1] | N-(4-{[(pyridin-3- ylmethyl)carbamoyl] amino}phenyl)-3′- (trifluoromethyl) biphenyl-2-sulfonamide | 527.1376 | 1, 2, 3, 4 |
| 451 | 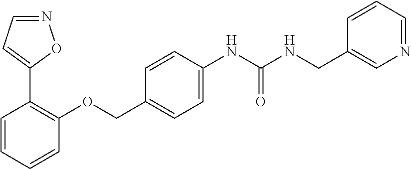 [SMILES: O=C(NCc1cccnc1)Nc1ccc(COc2ccccc2-c2ccno2)cc1] | 1-(4-{[2-(1,2- Oxazol-5- yl)phenoxy]methyl} phenyl)-3-(pyridin-3- ylmethyl)urea | 401.1653 | 7, 8 |
| 452 | 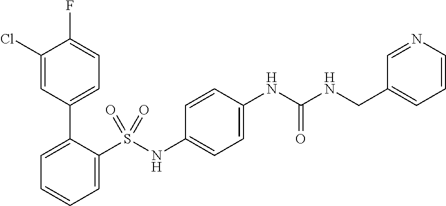 [SMILES: O=C(NCc1cccnc1)Nc1ccc(NS(=O)(=O)c2ccccc2-c2ccc(F)c(Cl)c2)cc1] | 3′-chloro-4′-fluoro- N-(4-{[(pyridin-3- ylmethyl)carbamoyl] amino}phenyl) biphenyl-2-sulfonamide | 511.1010 | 1, 2, 3, 4 |
| 453 | 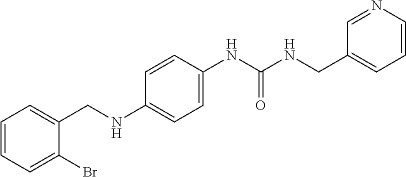 [SMILES: O=C(NCc1cccnc1)Nc1ccc(NCc2ccccc2Br)cc1] | 1-{4-[(2- Bromobenzyl)amino] phenyl}-3-(pyridin- 3-ylmethyl)urea | 411.0792 | 1, 2, 35 |
| 454 | 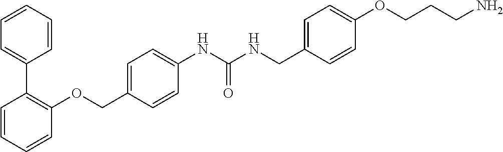 [SMILES: NCCCOc1ccc(CNC(=O)Nc2ccc(COc3ccccc3-c3ccccc3)cc2)cc1] | 1-[4-(3- Aminopropoxy)benzyl]- 3-{4-[(biphenyl-2- yloxy)methyl]phenyl} urea | 482.2416 | 44, 20, 46, 10, 32 |
| 455 | 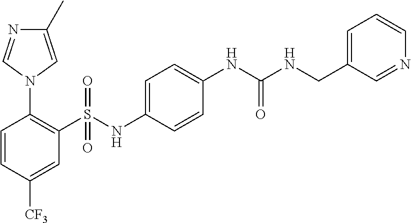 [SMILES: Cc1cn(-c2ccc(C(F)(F)F)cc2S(=O)(=O)Nc2ccc(NC(=O)NCc3cccnc3)cc2)cn1] | 2-(4-Methyl-1H- imidazol-1-yl)-N-(4- {[(pyridin-3- ylmethyl)carbamoyl] amino}phenyl)-5- (trifluoromethyl) benzenesulfonamide | 531.1403 | 17, 18, 76 |
| 456 | 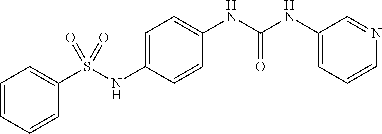 [SMILES: O=C(Nc1ccc(NS(=O)(=O)c2ccccc2)cc1)Nc1cccnc1] | N-{4-[(Pyridin-3- ylcarbamoyl)amino] phenyl} benzenesulfonamide | 369.1016 | 1, 2, 3 |
| 457 | 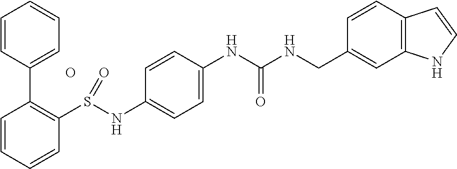 | N-(4-{[(1H-indol-6- ylmethyl)carbamoyl] amino}phenyl) biphenyl-2-sulfonamide | 497.1629 | 17, 18 |
| 458 | 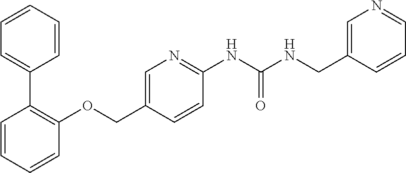 [SMILES: O=C(NCc1cccnc1)Nc1ccc(COc2ccccc2-c2ccccc2)cn1] | 1-{5-[(Biphenyl-2- yloxy)methyl]pyridin- 2-yl}-3-(pyridin-3- ylmethyl)urea | NA | 18, 10 |
| 459 | 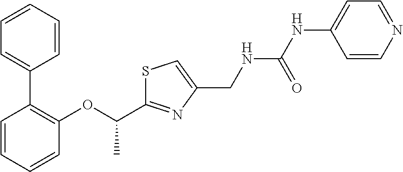 [SMILES: C[C@H](Oc1ccccc1-c1ccccc1)c1nc(CNC(=O)Nc2ccncc2)cs1] | 1-({2-[(1S)-1- (Biphenyl-2- yloxy)ethyl]-1,3- thiazol-4-yl}methyl)- 3-pyridin-4-ylurea | 431.1641 | 14, 15, 24, 25, 26, 18 |
| 460 | 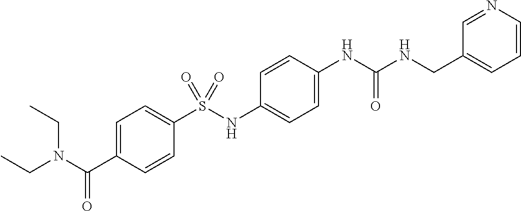 [SMILES: CCN(CC)C(=O)c1ccc(S(=O)(=O)Nc2ccc(NC(=O)NCc3cccnc3)cc2)cc1] | N,N-Diethyl-4-[(4- {[(pyridin-3- ylmethyl)carbamoyl] amino}phenyl)sulfamoyl] benzamide | 482.1836 | 1, 2, 3, 9 |
| 461 | 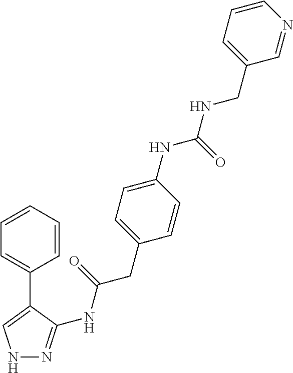 [SMILES: O=C(Cc1ccc(NC(=O)NCc2cccnc2)cc1)Nc1n[nH]cc1-c1ccccc1] | N-(4-Phenyl-1H- pyrazol-3-yl)-2-(4- {[(pyridin-3- ylmethyl)carbamoyl] amino}phenyl)acetamide | 427.2081 | 19, 33, 34 |
| 462 | 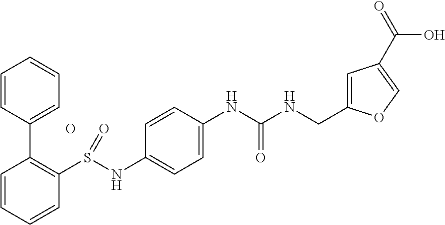 | 5-{[({4-[(biphenyl-2- ylsulfonyl)amino] phenyl}carbamoyl) amino] methyl}furan-3- carboxylic acid | 492.1221 | 17, 18 |
| 463 | 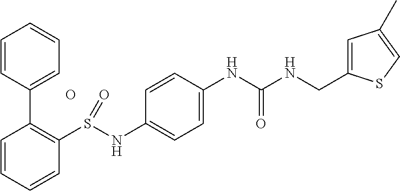 | N-[4-({[(4- methylthiophen-2- yl)methyl]carbamoyl} amino)phenyl]biphenyl- 2-sulfonamide | 478.1234 | 17, 18 |
| 464 | 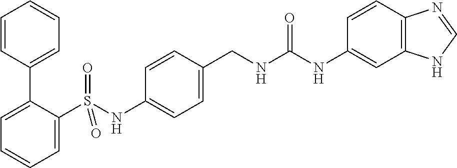 [SMILES: O=C(NCc1ccc(NS(=O)(=O)c2ccccc2-c2ccccc2)cc1)Nc1ccc2nc[nH]c2c1] | N-(4-{[(1H- benzimidazol-6- ylcarbamoyl)amino] methyl}phenyl)biphenyl- 2-sulfonamide | 498.1605 | 17, 18 |
| 465 | 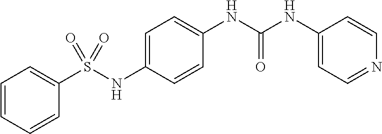 [SMILES: O=C(Nc1ccncc1)Nc1ccc(NS(=O)(=O)c2ccccc2)cc1] | N-{4-[(Pyridin-4- ylcarbamoyl)amino] phenyl} benzenesulfonamide | 369.1016 | 1, 2, 3 |
| 466 | 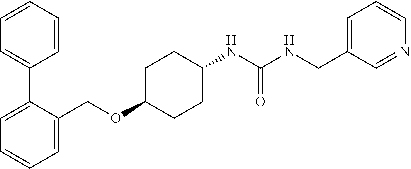 [SMILES: O=C(NCc1cccnc1)N[C@H]1CC[C@H](OCc2ccccc2-c2ccccc2)CC1] | 1-[trans-4-(Biphenyl- 2- ylmethoxy)cyclohexyl]- 3-(pyridin-3- ylmethyl)urea | 416.2348 | 58, 18, 59 |
| 467 | 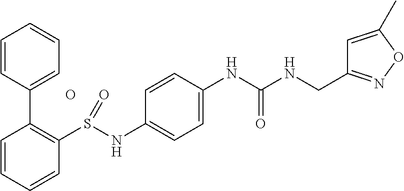 | N-[4-({[(5-methyl- 1,2-oxazol-3- yl)methyl]carbamoyl} amino)phenyl]biphenyl- 2-sulfonamide | 463.1433 | 17, 18 |
| 468 | 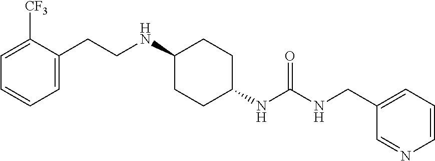 [SMILES: O=C(NCc1cccnc1)N[C@H]1CC[C@H](NCCc2ccccc2C(F)(F)F)CC1] | 1-(Pyridin-3- ylmethyl)-3-[trans-4- ({2-[2- (trifluoromethyl) phenyl]ethyl}amino) cyclohexyl]urea | 421.2251 | 35, 32, 46 |
| 469 | 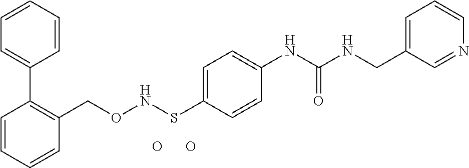 | N-(Biphenyl-2- ylmethoxy)-4- {[(pyridin-3- ylmethyl)carbamoyl] amino} benzenesulfonamide | 489.1610 | 82, 44, 18 |
| 470 | 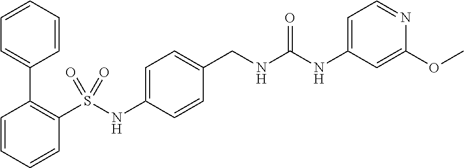 [SMILES: COc1cc(NC(=O)NCc2ccc(NS(=O)(=O)c3ccccc3-c3ccccc3)cc2)ccn1] | N-[4-({[(2- Methoxypyridin-4- yl)carbamoyl]amino} methyl)phenyl]biphenyl- 2-sulfonamide | 489.1617 | 5, 32, 18 |
| 471 | 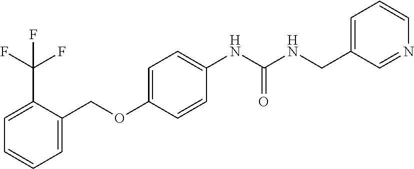 [SMILES: O=C(NCc1cccnc1)Nc1ccc(OCc2ccccc2C(F)(F)F)cc1] | 1-(Pyridin-3- ylmethyl)-3-(4-{[2- (trifluoromethyl) benzyl]oxy}phenyl)urea | 402.1427 | 10, 56, 18 |
| 472 | 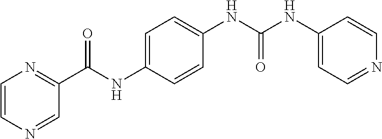 [SMILES: O=C(Nc1ccncc1)Nc1ccc(NC(=O)c2cnccn2)cc1] | N-{4-[(Pyridin-4- ylcarbamoyl)amino] phenyl}pyrazine-2- carboxamide | 335.1311 | 1, 2, 40 |
| 473 | 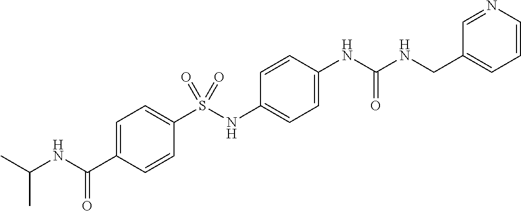 [SMILES: CC(C)NC(=O)c1ccc(S(=O)(=O)Nc2ccc(NC(=O)NCc3cccnc3)cc2)cc1] | N-(Propan-2-yl)-4- [(4-{[(pyridin-3- ylmethyl)carbamoyl] amino}phenyl)sulfamoyl] benzamide | 468.1757 | 1, 2, 3, 9 |
| 474 | 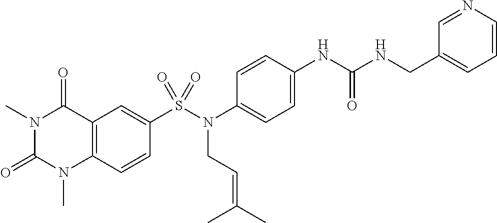 [SMILES: CC(C)=CCN(c1ccc(NC(=O)NCc2cccnc2)cc1)S(=O)(=O)c1ccc2c(c1)c(=O)n(C)c(=O)n2C] | 1,3-dimethyl-N-(3- methylbut-2-en-1- yl)-2,4-dioxo-N-(4- {[(pyridin-3- ylmethyl)carbamoyl] amino}phenyl)- 1,2,3,4- tetrahydroquinazoline- 6-sulfonamide | 563.2065 | 1, 2, 3, 19 |
| 475 | 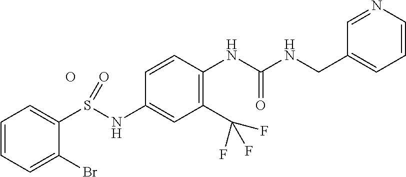 | 2-bromo-N-[4- {[(pyridin-3- ylmethyl)carbamoyl] amino}-3- (trifluoromethyl)phenyl] benzenesulfonamide | 529.0164 | 1, 2, 3 |
| 476 | 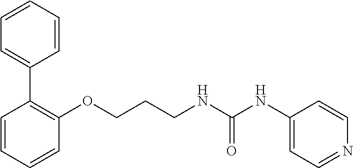 [SMILES: O=C(NCCCOc1ccccc1-c1ccccc1)Nc1ccncc1] | 1-[3-(biphenyl-2- yloxy)propyl]-3- pyridin-4-ylurea | 348.1759 | 10, 18 |
| 477 | 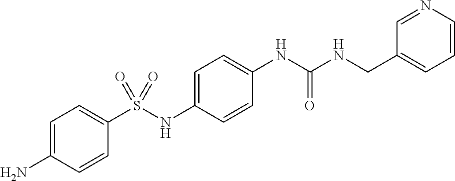 [SMILES: Nc1ccc(S(=O)(=O)Nc2ccc(NC(=O)NCc3cccnc3)cc2)cc1] | 4-Amino-N-(4- {[(pyridin-3- ylmethyl)carbamoyl] amino}phenyl) benzenesulfonamide | 398.1245 | 1, 2, 3, 2 |
| 478 | 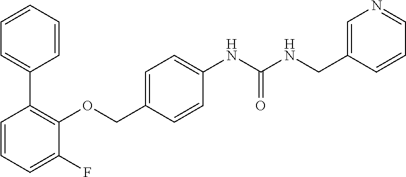 [SMILES: O=C(NCc1cccnc1)Nc1ccc(COc2c(F)cccc2-c2ccccc2)cc1] | 1-(4-{[(3- Fluorobiphenyl-2- yl)oxy]methyl}phenyl)- 3-(pyridin-3- ylmethyl)urea | 428.1773 | 7, 8, 4 |
| 479 | 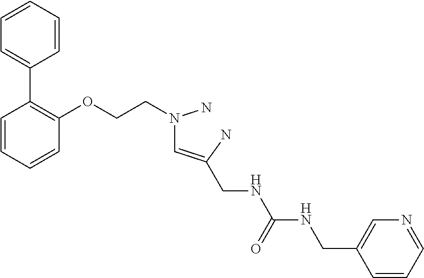 [SMILES: N/C(=C\N(N)CCOc1ccccc1-c1ccccc1)CNC(=O)NCc1cccnc1] | 1-({1-[2-(Biphenyl- 2-yloxy)ethyl]-1H- 1,2,3-triazol-4- yl}methyl)-3- (pyridin-3- ylmethyl)urea | 429.1989 | 8, 83, 84, 32, 6 |
| 480 | 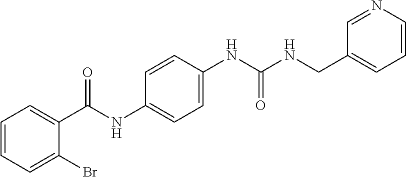 [SMILES: O=C(NCc1cccnc1)Nc1ccc(NC(=O)c2ccccc2Br)cc1] | 2-Bromo-N-(4- {[(pyridin-3- ylmethyl)carbamoyl] amino}phenyl) benzamide | 425.0597 | 1, 2, 9 |
| 481 | 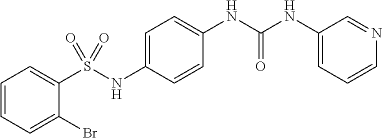 [SMILES: O=C(Nc1ccc(NS(=O)(=O)c2ccccc2Br)cc1)Nc1cccnc1] | 2-Bromo-N-{4- [(pyridin-3- ylcarbamoyl)amino] phenyl} benzenesulfonamide | 447.0123 | 1, 2, 3 |
| 482 | 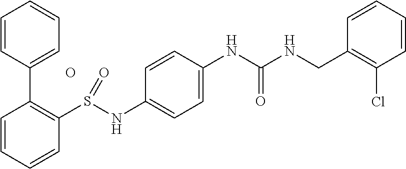 | N-(4-{[(2- chlorobenzyl)carbamoyl] amino}phenyl) biphenyl-2- sulfonamide | 492.1138 | 17, 18 |
| 483 | 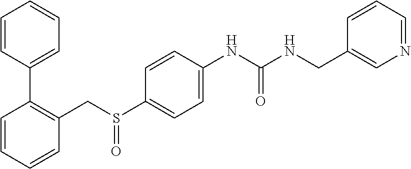 [SMILES: O=C(NCc1cccnc1)Nc1ccc(S(=O)Cc2ccccc2-c2ccccc2)cc1] | 1-{4-[(biphenyl-2- ylmethyl)sulfinyl] phenyl}-3-(pyridin-3- ylmethyl)urea | | 13, 2, 4, 18, 12 |
| 484 | 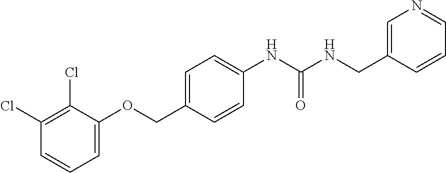 [SMILES: O=C(NCc1cccnc1)Nc1ccc(COc2cccc(Cl)c2Cl)cc1] | 1-{4-[(2,3- Dichlorophenoxy) methyl]phenyl}-3- (pyridin-3- ylmethyl)urea | 402.0777 | 7, 8 |
| 485 | 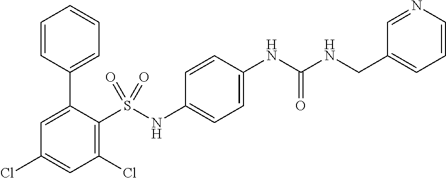 [SMILES: O=C(NCc1cccnc1)Nc1ccc(NS(=O)(=O)c2c(Cl)cc(Cl)cc2-c2ccccc2)cc1] | 3,5-dichloro-N-(4- {[(pyridin-3- ylmethyl)carbamoyl] amino}phenyl)biphenyl- 2-sulfonamide | 527.0673 | 1, 2, 3, 4 |
| 486 | 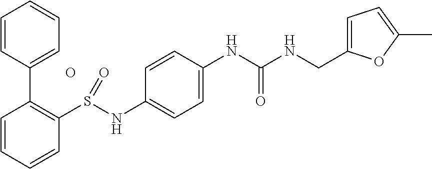 | N-[4-({[(5- methylfuran-2- yl)methyl]carbamoyl} amino)phenyl]biphenyl- 2-sulfonamide | 462.1484 | 17, 18 |
| 487 | 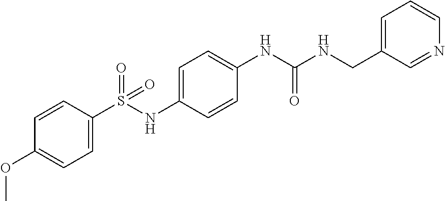 [SMILES: COc1ccc(S(=O)(=O)Nc2ccc(NC(=O)NCc3cccnc3)cc2)cc1] | 4-Methoxy-N-(4- {[(pyridin-3- ylmethyl)carbamoyl] amino}phenyl) benzenesulfonamide | 413.1273 | 1, 2, 3 |
| 488 | 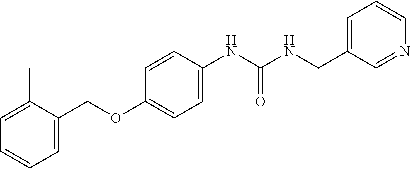 [SMILES: Cc1ccccc1COc1ccc(NC(=O)NCc2cccnc2)cc1] | 1-{4-[(2- Methylbenzyl)oxy] phenyl}-3-(pyridin-3- ylmethyl)urea | 348.1670 | 44, 56, 18 |
| 489 | 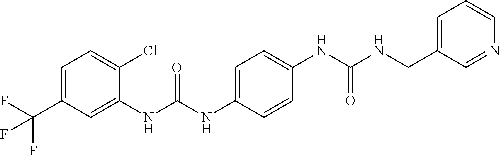 [SMILES: O=C(NCc1cccnc1)Nc1ccc(NC(=O)Nc2cc(C(F)(F)F)ccc2Cl)cc1] | 3-[2-Chloro-5- (trifluoromethyl)phenyl]- 3-(4-{[(pyridin-3- ylmethyl)carbamoyl] amino}phenyl)urea (non-preferred name) | 464.0893 | 1, 2, 18 |
| 490 | 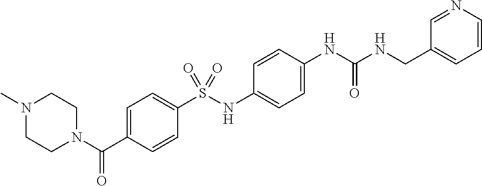 [SMILES: CN1CCN(C(=O)c2ccc(S(=O)(=O)Nc3ccc(NC(=O)NCc4cccnc4)cc3)cc2)CC1] | 4-[(4- Methylpiperazin-1- yl)carbonyl]-N-(4- {[(pyridin-3- ylmethyl)carbamoyl] amino}phenyl) benzenesulfonamide | 509.2082 | 1, 2, 3, 9 |
| 491 | 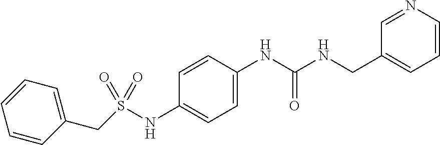 [SMILES: O=C(NCc1cccnc1)Nc1ccc(NS(=O)(=O)Cc2ccccc2)cc1] | 1-Phenyl-N-(4- {[(pyridin-3- ylmethyl)carbamoyl] amino}phenyl) methanesulfonamide | 397.1363 | 1, 2, 3 |
| 492 | 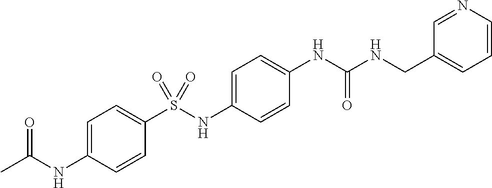 [SMILES: CC(=O)Nc1ccc(S(=O)(=O)Nc2ccc(NC(=O)NCc3cccnc3)cc2)cc1] | N-{4-[(4-{[(Pyridin- 3- ylmethyl)carbamoyl] amino}phenyl)sulfamoyl] phenyl}acetamide | 440.1432 | 1, 2, 3, 9 |
| 493 | 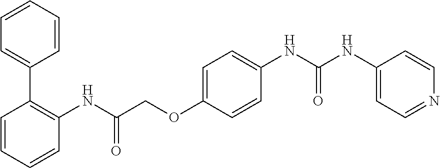 [SMILES: O=C(COc1ccc(NC(=O)Nc2ccncc2)cc1)Nc1ccccc1-c1ccccc1] | N-(Biphenyl-2-yl)-2- {4-[(pyridin-4- ylcarbamoyl)amino] phenoxy}acetamide | 439.1685 | 40, 10, 18 |
| 494 | 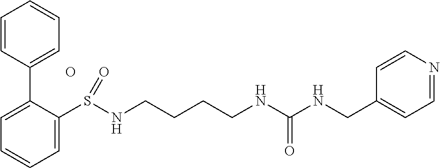 | N-(4-{[(pyridin-4- ylmethyl)carbamoyl] amino}butyl)biphenyl- 2-sulfonamide | 439.1799 | 17, 18 |
| 495 | 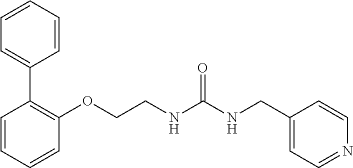 [SMILES: O=C(NCCOc1ccccc1-c1ccccc1)NCc1ccncc1] | 1-[2-(Biphenyl-2- yloxy)ethyl]-3- (pyridin-4- ylmethyl)urea | 370.15731 [M + Na]+ | 10, 18 |
| 496 | 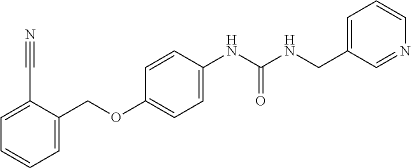 [SMILES: N#Cc1ccccc1COc1ccc(NC(=O)NCc2cccnc2)cc1] | 1-{4-[(2- Cyanobenzyl)oxy] phenyl}-3-(pyridin-3- ylmethyl)urea | 359.1530 | 44, 32, 18 |
| 497 | 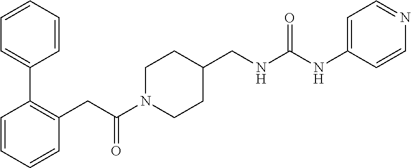 [SMILES: O=C(NCC1CCN(C(=O)Cc2ccccc2-c2ccccc2)CC1)Nc1ccncc1] | 1-{[1-(Biphenyl-2- ylacetyl)piperidin-4- yl]methyl}-3- pyridin-4-ylurea | 429.2329 | 18, 32, 9, 4 |
| 498 | 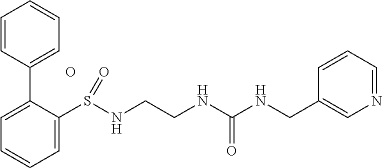 | N-(2-{[(pyridin-3- ylmethyl)carbamoyl] amino}ethyl)biphenyl- 2-sulfonamide | 411.1475 | 18, 39, 3 |
| 499 | 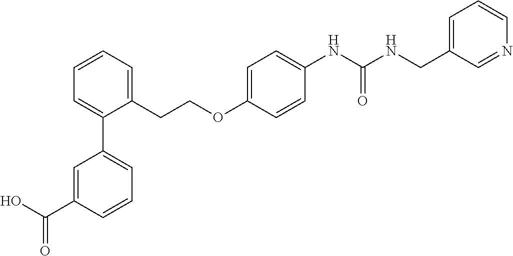 [SMILES: O=C(NCc1cccnc1)Nc1ccc(OCCc2ccccc2-c2cccc(C(=O)O)c2)cc1] | 2′-[2-(4-{[(Pyridin-3- ylmethyl)carbamoyl] amino}phenoxy)ethyl] biphenyl-3- carboxylic acid | 468- 1936 | 10, 20, 46, 4 |
| 500 | 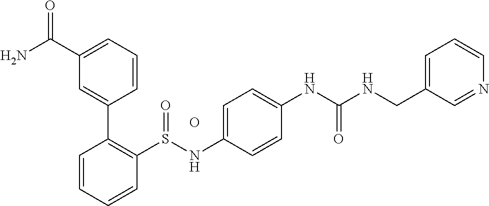 | 2′-[(4-{[(Pyridin-3- ylmethyl)carbamoyl] amino}phenyl)sulfamoyl] biphenyl-3- carboxamide | 502.1610 | 1, 2, 3, 4 |
| 501 | 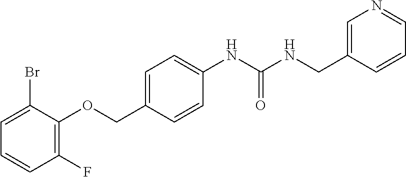 [SMILES: O=C(NCc1cccnc1)Nc1ccc(COc2c(F)cccc2Br)cc1] | 1-{4-[(2-Bromo-6- fluorophenoxy)methyl] phenyl}-3-(pyridin- 3-ylmethyl)urea | 430.0561 | 7, 8 |
| 502 | 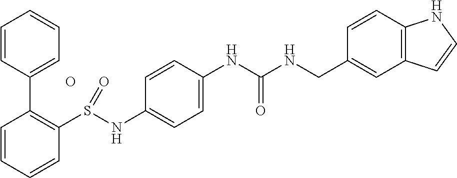 | N-(4-{[(1H-indol-5- ylmethyl)carbamoyl] amino}phenyl)biphenyl- 2-sulfonamide | 497.1628 | 17, 18 |
| 503 | 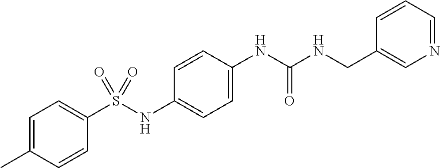 [SMILES: Cc1ccc(S(=O)(=O)Nc2ccc(NC(=O)NCc3cccnc3)cc2)cc1] | 4-methyl-N-(4- {[(pyridin-3- ylmethyl)carbamoyl] amino}phenyl) benzenesulfonamide | 397.1342 | 18, 3 |
| 504 | 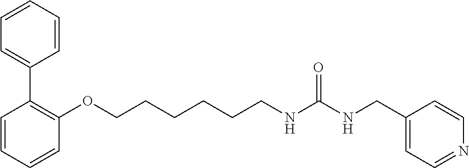 [SMILES: O=C(NCCCCCCOc1ccccc1-c1ccccc1)NCc1ccncc1] | 1-[6-(Biphenyl-2- yloxy)hexyl]-3- (pyridin-4- ylmethyl)urea | 404.2331 | 10, 18 |
| 505 | 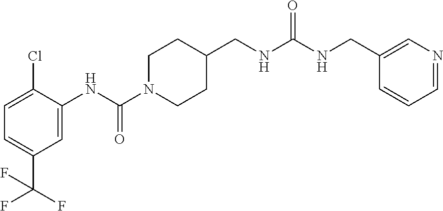 [SMILES: O=C(NCc1cccnc1)NCC1CCN(C(=O)Nc2cc(C(F)(F)F)ccc2Cl)CC1] | N-[2-Chloro-5- (trifluoromethyl)phenyl]- 4-({[(pyridin-3- ylmethyl)carbamoyl] amino}methyl)piperidine- 1-carboxamide | 470.1631 | 18, 32, 18 |
| 506 | 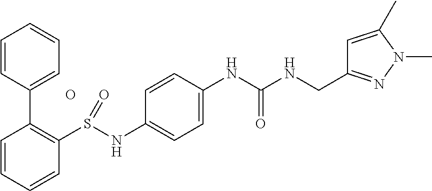 | N-[4-({[(1,5- dimethyl-1H- pyrazol-3- yl)methyl]carbamoyl} amino)phenyl] biphenyl-2-sulfonamide | 476.1745 | 17, 18 |
| 507 | 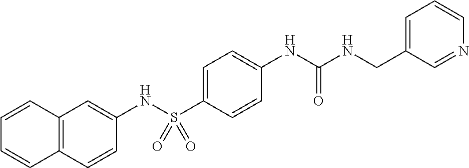 [SMILES: O=C(NCc1cccnc1)Nc1ccc(S(=O)(=O)Nc2ccc3ccccc3c2)cc1] | N-(Naphthalen-2-yl)- 4-{[(pyridin-3- ylmethyl)carbamoyl] amino} benzenesulfonamide | 433.1336 | 10, 2, 18 |
| 508 | 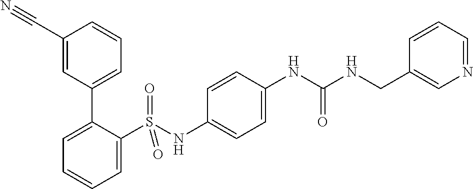 [SMILES: N#Cc1cccc(-c2ccccc2S(=O)(=O)Nc2ccc(NC(=O)NCc3cccnc3)cc2)c1] | 3′-cyano-N-(4- {[(pyridin-3- ylmethyl)carbamoyl] amino}phenyl)biphenyl- 2-sulfonamide | 484.1405 | 1, 2, 3, 4 |
| 509 | 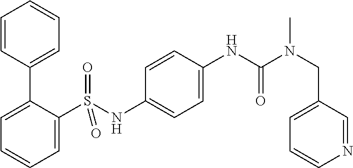 [SMILES: CN(Cc1cccnc1)C(=O)Nc1ccc(NS(=O)(=O)c2ccccc2-c2ccccc2)cc1] | N-(4- {[methyl(pyridin-3- ylmethyl)carbamoyl] amino}phenyl)biphenyl- 2-sulfonamide | 473.2262 | 17, 18 |
| 510 | 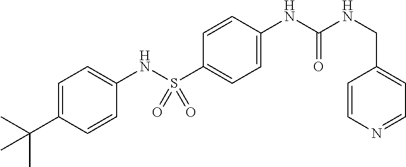 [SMILES: CC(C)(C)c1ccc(NS(=O)(=O)c2ccc(NC(=O)NCc3ccncc3)cc2)cc1] | N-(4-tert- Butylphenyl)-4- {[(pyridin-4- ylmethyl)carbamoyl] amino} benzenesulfonamide | 439.1813 | 5, 2, 18 |
| 511 | 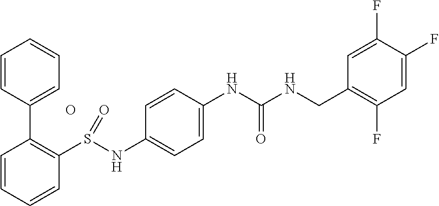 | N-(4-{[(2,4,5- trifluorobenzyl) carbamoyl]amino}phenyl) biphenyl-2- sulfonamide | 512.1259 | 17, 18 |
| 512 | 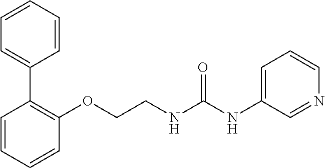 [SMILES: O=C(NCCOc1ccccc1-c1ccccc1)Nc1cccnc1] | 1-[2-(Biphenyl-2- yloxy)ethyl]-3- pyridin-3-ylurea | 334.3548 | 10, 18 |
| 513 | 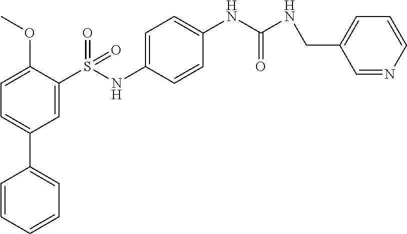 [SMILES: COc1ccc(-c2ccccc2)cc1S(=O)(=O)Nc1ccc(NC(=O)NCc2cccnc2)cc1] | 4-Methoxy-N-(4- {[(pyridin-3- amino}phenyl)biphenyl- 3-sulfonamide | 489.1584 | 1, 2, 3, 4 |
| 514 | 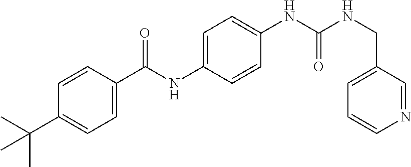 [SMILES: CC(C)(C)c1ccc(C(=O)Nc2ccc(NC(=O)NCc3cccnc3)cc2)cc1] | 4-tert-butyl-N-(4- {[(pyridin-3- ylmethyl)carbamoyl] amino}phenyl)benzamide | 403.2129 | 1, 2, 9 |
| 515 | 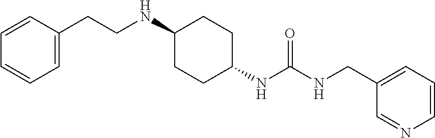 [SMILES: O=C(NCc1cccnc1)N[C@H]1CC[C@H](NCCc2ccccc2)CC1] | 1-{trans-4-[(2- Phenylethyl)amino] cyclohexyl}-3- (pyridin-3- ylmethyl)urea | 353.2384 | 35, 32, 46, 71 |
| 516 | 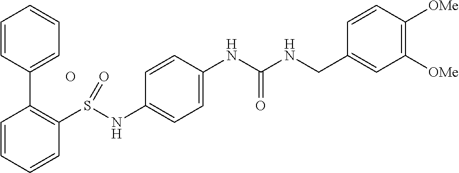 | N-(4-{[(3,4- dimethoxybenzyl) carbamoyl]amino} phenyl)biphenyl-2- sulfonamide | 518.1745 | 17, 18 |
| 517 | 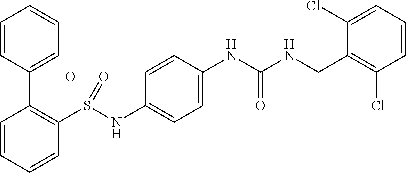 | N-(4-{[(2,6- dichlorobenzyl) carbamoyl] amino}phenyl) biphenyl-2- sulfonamide | 526.0747 | 17, 18 |
| 518 | 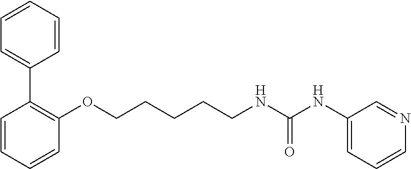 [SMILES: O=C(NCCCCCOc1ccccc1-c1ccccc1)Nc1cccnc1] | 1-[5-(Biphenyl-2- yloxy)pentyl]-3- pyridin-3-ylurea | 376.2110 | 10, 18 |
| 519 | 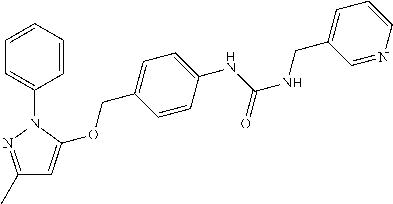 [SMILES: Cc1cc(OCc2ccc(NC(=O)NCc3cccnc3)cc2)n(-c2ccccc2)n1] | 1-(4-{[(3-Methyl-1- phenyl-1H-pyrazol- 5- yl)oxy]methyl}phenyl)- 3-(pyridin-3- ylmethyl)urea | 414.1912 | 7, 8 |
| 520 | 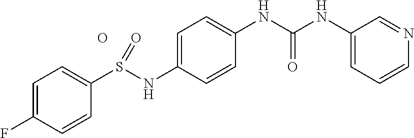 | 4-Fluoro-N-{4- [(pyridin-3- ylcarbamoyl)amino] phenyl} benzenesulfonamide | 387.1003 | 1, 2, 3 |
| 521 | 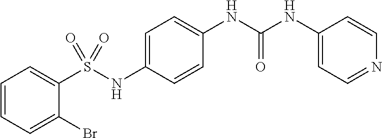 [SMILES: O=C(Nc1ccncc1)Nc1ccc(NS(=O)(=O)c2ccccc2Br)cc1] | 2-Bromo-N-{4- [(pyridin-4- ylcarbamoyl)amino] phenyl} benzenesulfonamide | 449.0101 (M + 2H) | 1, 2, 3 |
| 522 | 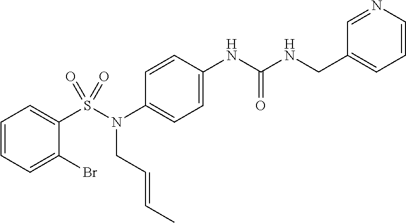 [SMILES: C/C=C/CN(c1ccc(NC(=O)NCc2cccnc2)cc1)S(=O)(=O)c1ccccc1Br] | 2-Bromo-N-[(2E)- but-2-en-1-yl]-N-(4- {[(pyridin-3- ylmethyl)carbamoyl] amino}phenyl) benzenesulfonamide | 515.0724 | 1, 2, 3, 19 |
| 523 | 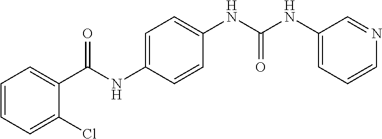 [SMILES: O=C(Nc1ccc(NC(=O)c2ccccc2Cl)cc1)Nc1cccnc1] | 2-Chloro-N-{4- [(pyridin-3- ylcarbamoyl)amino] phenyl}benzamide | 367.0983 | 1, 2, 40 |
| 524 | 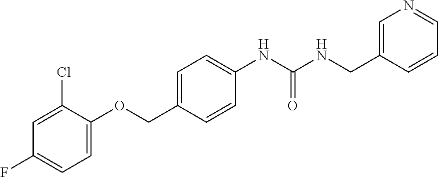 [SMILES: O=C(NCc1cccnc1)Nc1ccc(COc2ccc(F)cc2Cl)cc1] | 1-{4-[(2-Chloro-4- fluorophenoxy)methyl] phenyl}-3-(pyridin- 3-ylmethyl)urea | 386.1075 | 7, 8 |
| 525 | 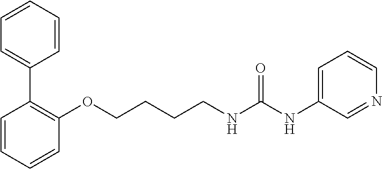 [SMILES: O=C(NCCCCOc1ccccc1-c1ccccc1)Nc1cccnc1] | 1-[4-(Biphenyl-2- yloxy)butyl]-3- pyridin-3-ylurea | 362.1948 | 10, 18 |
| 526 | 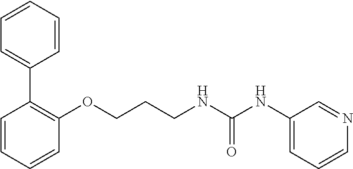 [SMILES: O=C(NCCCOc1ccccc1-c1ccccc1)Nc1cccnc1] | 1-[3-(Biphenyl-2- yloxy)propyl]-3- pyridin-3-ylurea | 348.1772 | 10, 18 |
| 527 | 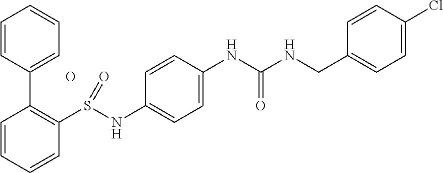 | N-(4-{[(4- chlorobenzyl)carbamoyl] amino}phenyl) biphenyl-2- sulfonamide | 492.1140 | 17, 18 |
| 528 | 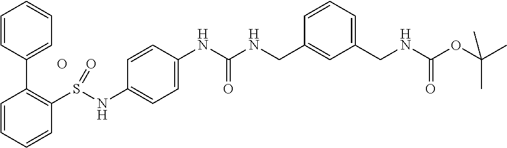 | tert-butyl (3-{[({4- [(biphenyl-2- ylsulfonyl)amino] phenyl}carbamoyl) amino] methyl}benzyl) carbamate | 609.20015 [M + Na+] | 17, 18 |
| 529 | 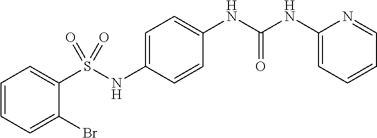 [SMILES: O=C(Nc1ccc(NS(=O)(=O)c2ccccc2Br)cc1)Nc1ccccn1] | 2-Bromo-N-{4- [(pyridin-2- ylcarbamoyl)amino] phenyl} benzenesulfonamide | 447.0165 | 1, 2, 3 |
| 530 | 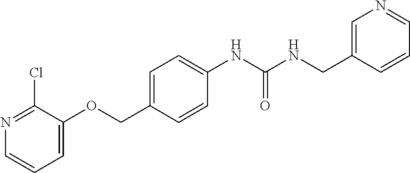 [SMILES: O=C(NCc1cccnc1)Nc1ccc(COc2cccnc2Cl)cc1] | 1-(4-{[(2- Chloropyridin-3- yl)oxy]methyl}phenyl)- 3-(pyridin-3- ylmethyl)urea | 369.1114 | 10, 18 |
| 531 | 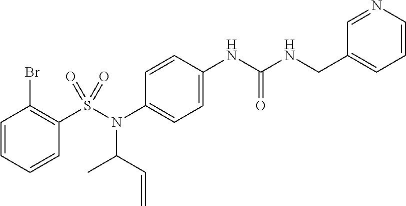 [SMILES: C=CC(C)N(c1ccc(NC(=O)NCc2cccnc2)cc1)S(=O)(=O)c1ccccc1Br] | 2-Bromo-N-(but-3- en-2-yl)-N-(4- {[(pyridin-3- ylmethyl)carbamoyl] amino}phenyl) benzenesulfonamide | 515.0723 | 1, 2, 3, 19 |
| 532 | 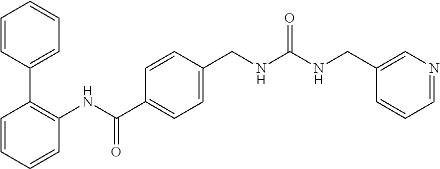 [SMILES: O=C(NCc1ccc(C(=O)Nc2ccccc2-c2ccccc2)cc1)NCc1cccnc1] | N-(biphenyl-2-yl)-4- ({[(pyridin-3- ylmethyl)carbamoyl] amino}methyl)benzamide | 437.2000 | 9, 32, 18 |
| 533 | 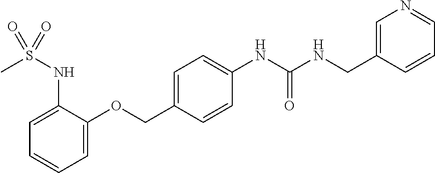 [SMILES: CS(=O)(=O)Nc1ccccc1OCc1ccc(NC(=O)NCc2cccnc2)cc1] | N-{2-[(4-{[(Pyridin- 3- ylmethyl)carbamoyl] amino}benzyl)oxy] phenyl} methanesulfonamide | 427.1472 | 65, 7, 8, 32 |
| 534 | 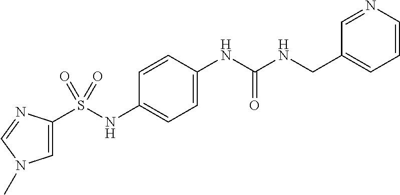 [SMILES: Cn1cnc(S(=O)(=O)Nc2ccc(NC(=O)NCc3cccnc3)cc2)c1] | 1-methyl-N-(4- {[(pyridin-3- ylmethyl)carbamoyl] amino}phenyl)-1H- imidazole-4- sulfonamide | 387.1236 | 1, 2, 3 |
| 535 | 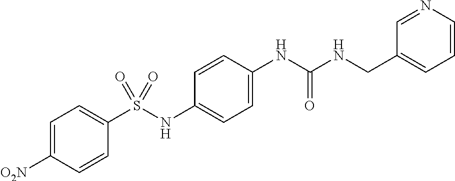 [SMILES: O=C(NCc1cccnc1)Nc1ccc(NS(=O)(=O)c2ccc([N+](=O)[O-])cc2)cc1] | 4-Nitro-N-(4- {[(pyridin-3- ylmethyl)carbamoyl] amino}phenyl) benzenesulfonamide | 428.1024 | 1, 2, 3 |
| 536 | 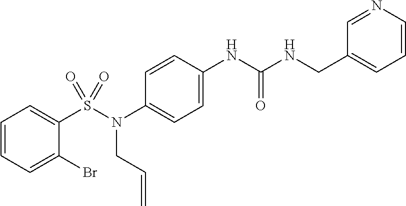 [SMILES: C=CCN(c1ccc(NC(=O)NCc2cccnc2)cc1)S(=O)(=O)c1ccccc1Br] | 2-Bromo-N-(prop-2- en-1-yl)-N-(4- {[(pyridin-3- ylmethyl)carbamoyl] amino}phenyl) benzenesulfonamide | 501.0579 | 1, 2, 3, 19 |
| 537 | 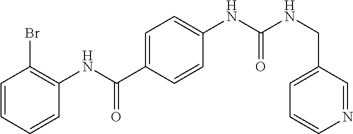 [SMILES: O=C(NCc1cccnc1)Nc1ccc(C(=O)Nc2ccccc2Br)cc1] | N-(2-bromophenyl)- 4-{[(pyridin-3- ylmethyl)carbamoyl] amino}benzamide | 425.0627 | 1, 16, 9 |
| 538 | 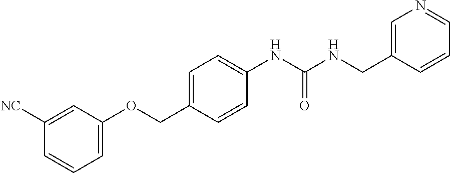 [SMILES: N#Cc1cccc(OCc2ccc(NC(=O)NCc3cccnc3)cc2)c1] | 1-{4-[(3- Cyanophenoxy) methyl]phenyl}-3- (pyridin-3- ylmethyl)urea | 359.1505 | 7, 8 |
| 539 | 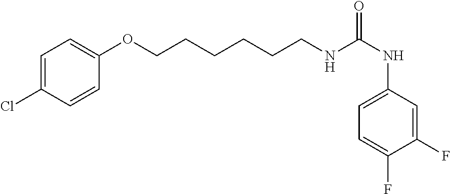 [SMILES: O=C(NCCCCCCOc1ccc(Cl)cc1)Nc1ccc(F)c(F)c1] | 1-[6-(4- Chlorophenoxy)hexyl]- 3-(3,4- difluorophenyl)urea | 383.1330 | 41, 42, 47 |
| 540 | 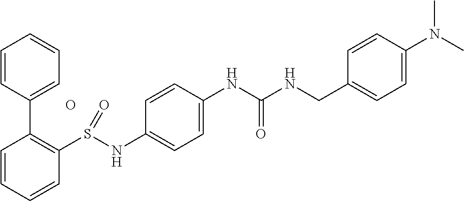 | N-[4-({[4- (dimethylamino)benzyl] carbamoyl}amino) phenyl]biphenyl-2- sulfonamide | 501.1976 | 17, 18 |
| 541 | 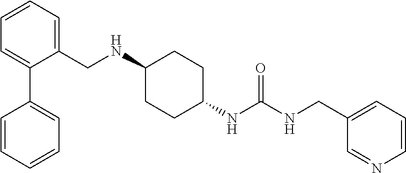 [SMILES: O=C(NCc1cccnc1)N[C@H]1CC[C@H](NCc2ccccc2-c2ccccc2)CC1] | 1-{trans-4- [(Biphenyl-2- ylmethyl)amino] cyclohexyl}-3-(pyridin- 3-ylmethyl)urea | 415.2497 | 67, 32, 46, 71 |
| 542 | 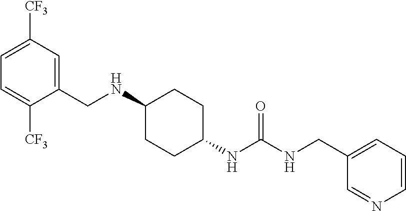 [SMILES: O=C(NCc1cccnc1)N[C@H]1CC[C@H](NCc2cc(C(F)(F)F)ccc2C(F)(F)F)CC1] | 1-(trans-4-{[2,5- Bis(trifluoromethyl) benzyl]amino} cyclohexyl)- 3-(pyridin-3- ylmethyl)urea | 475.1936 | 67, 32, 46, 71 |
| 543 | 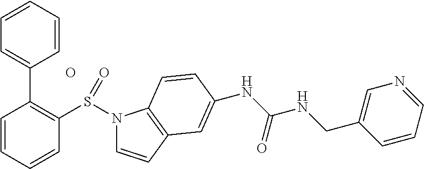 | 1-[1-(Biphenyl-2- ylsulfonyl)-1H-indol- 5-yl]-3-(pyridin-3- ylmethyl)urea | 483.1476 | 46, 3, 45 |
| 544 | 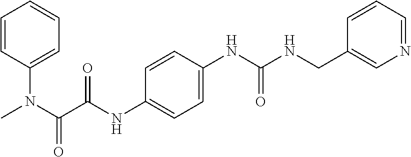 [SMILES: CN(C(=O)C(=O)Nc1ccc(NC(=O)NCc2cccnc2)cc1)c1ccccc1] | N-methyl-N-phenyl- N′-(4-{[(pyridin-3- ylmethyl)carbamoyl] amino}phenyl) ethanediamide | 404.1689 | 18, 23 |
| 545 | 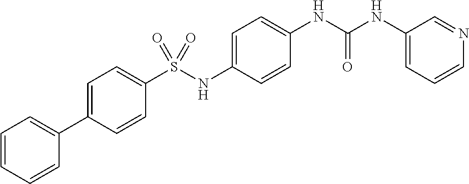 [SMILES: O=C(Nc1ccc(NS(=O)(=O)c2ccc(-c3ccccc3)cc2)cc1)Nc1cccnc1] | N-{4-[(Pyridin-3- ylcarbamoyl)amino] phenyl}biphenyl-4- sulfonamide | 445.1329 | 1, 2, 3 |
| 546 | 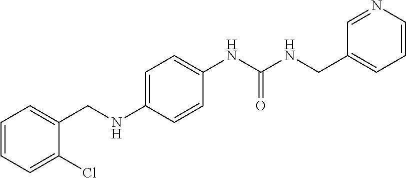 [SMILES: O=C(NCc1cccnc1)Nc1ccc(NCc2ccccc2Cl)cc1] | 1-{4-[(2- Chlorobenzyl)amino] phenyl}-3-(pyridin- 3-ylmethyl)urea | 367.1311 | 1, 2, 35 |
| 547 | 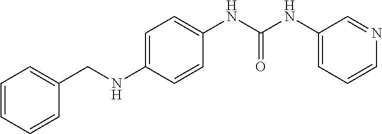 [SMILES: O=C(Nc1ccc(NCc2ccccc2)cc1)Nc1cccnc1] | 1-[4- (Benzylamino)phenyl]- 3-pyridin-3-ylurea | 319.1643 | 1, 2, 35 |
| 548 | 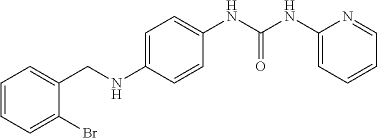 [SMILES: O=C(Nc1ccc(NCc2ccccc2Br)cc1)Nc1ccccn1] | 1-{4-[(2- Bromobenzyl)amino] phenyl}-3-pyridin-2- ylurea | 397.0705 | 1, 2, 35 |
| 549 | 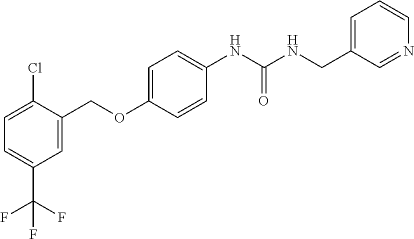 [SMILES: O=C(NCc1cccnc1)Nc1ccc(OCc2cc(C(F)(F)F)ccc2Cl)cc1] | 1-(4-{[2-Chloro-5- (trifluoromethyl)benzyl] oxy}phenyl)-3- (pyridin-3- ylmethyl)urea | 436.1070 | 44, 32, 18 |
| 550 | 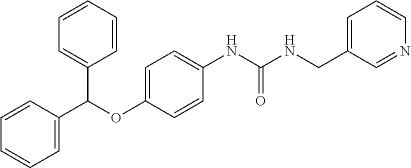 [SMILES: O=C(NCc1cccnc1)Nc1ccc(OC(c2ccccc2)c2ccccc2)cc1] | 1-[4- (Diphenylmethoxy) phenyl]-3-(pyridin-3- ylmethyl)urea | 410.1865 | 44, 32, 18 |
| 551 | 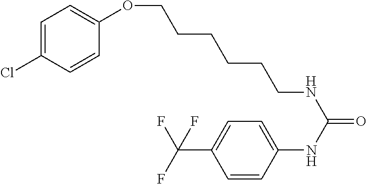 [SMILES: O=C(NCCCCCCOc1ccc(Cl)cc1)Nc1ccc(C(F)(F)F)cc1] | 1-[6-(4- Chlorophenoxy)hexyl]- 3-[4- (trifluoromethyl) phenyl]urea | 415.1398 | 41, 42, 47 |
| 552 | 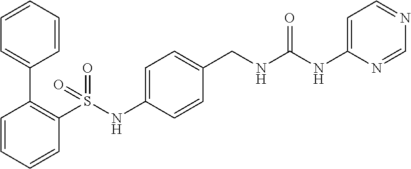 [SMILES: O=C(NCc1ccc(NS(=O)(=O)c2ccccc2-c2ccccc2)cc1)Nc1ccncn1] | N-(4-{[(pyrimidin-4- ylcarbamoyl)amino] methyl}phenyl)biphenyl- 2-sulfonamide | 460.1442 | 17, 18 |
| 553 | 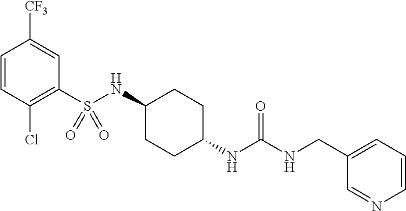 [SMILES: O=C(NCc1cccnc1)N[C@H]1CC[C@H](NS(=O)(=O)c2cc(C(F)(F)F)ccc2Cl)CC1] | 2-Chloro-N-(trans-4- {[(pyridin-3- ylmethyl)carbamoyl] amino}cyclohexyl)- 5- (trifluoromethyl) benzenesulfonamide | 491.1118 | 3, 32, 46 |
| 554 | 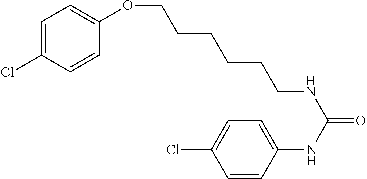 [SMILES: O=C(NCCCCCCOc1ccc(Cl)cc1)Nc1ccc(Cl)cc1] | 1-[6-(4- Chlorophenoxy) hexyl]-3-(4- chlorophenyl)urea | 381.1134 | 41, 42, 47 |
| 555 | 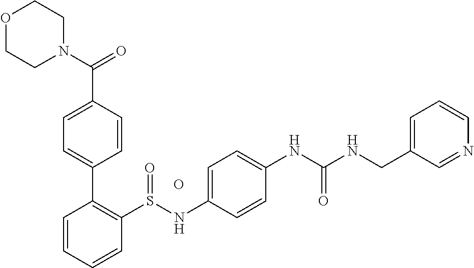 | 4′-(Morpholin-4- ylcarbonyl)-N-(4- {[(pyridin-3- ylmethyl)carbamoyl] amino}phenyl)biphenyl- 2-sulfonamide | 572.2026 | 1, 2, 3, 4 |
| 556 | 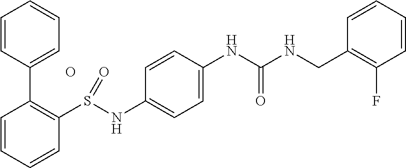 | N-(4-{[(2- fluorobenzyl)carbamoyl] amino}phenyl) biphenyl-2- sulfonamide | 502.1439 | 17, 18 |
| 557 | 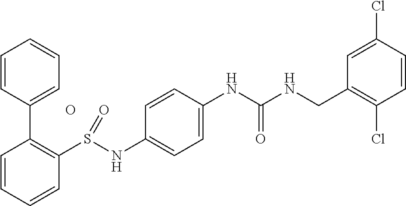 | N-(4-{[(2,5- dichlorobenzyl) carbamoyl] amino}phenyl) biphenyl-2- sulfonamide | 526.0741 | 17, 18 |
| 558 | 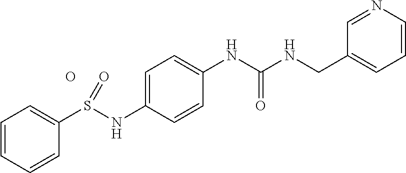 | N-(4-{[(Pyridin-3- ylmethyl)carbamoyl] amino}phenyl) benzenesulfonamide | 383.1210 | 1, 2, 3 |
| 559 | 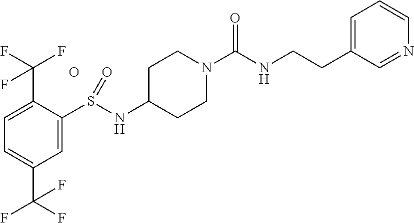 | 4-({[2,5- Bis(trifluoromethyl) phenyl]sulfonyl}amino)- N-[2-(pyridin-3- yl)ethyl]piperidine-1- carboxamide | 525.1269 | 3, 32, 18 |
| 560 | 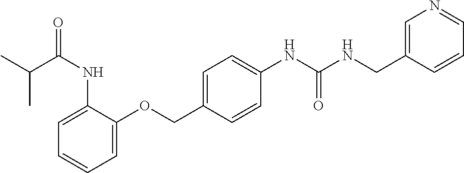 [SMILES: CC(C)C(=O)Nc1ccccc1OCc1ccc(NC(=O)NCc2cccnc2)cc1] | 2-Methyl-N-{2-[(4- {[(pyridin-3- ylmethyl)carbamoyl] amino}benzyl)oxy] phenyl}propanamide | 419.2072 | 65, 7, 8, 32 |
| 561 | 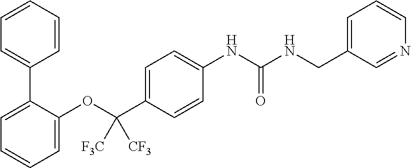 [SMILES: O=C(NCc1cccnc1)Nc1ccc(C(Oc2ccccc2-c2ccccc2)(C(F)(F)F)C(F)(F)F)cc1] | 1-{4-[2-(Biphenyl-2- yloxy)-1,1,1,3,3,3- hexafluoropropan-2- yl]phenyl}-3- (pyridin-3- ylmethyl)urea | 546.1606 | 10, 18 |
| 562 | 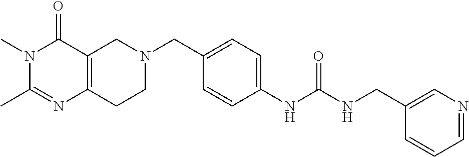 [SMILES: Cc1nc2c(c(=O)n1C)CN(Cc1ccc(NC(=O)NCc3cccnc3)cc1)CC2] | 1-{4-[(2,3-Dimethyl- 4-oxo-3,5,7,8- tetrahydropyrido[4,3- d]pyrimidin-6(4H)- yl)methyl]phenyl}-3- (pyridin-3- ylmethyl)urea | | 30, 31, 32, 48, 2, 18 |
| 563 | 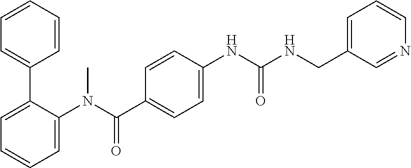 [SMILES: CN(C(=O)c1ccc(NC(=O)NCc2cccnc2)cc1)c1ccccc1-c1ccccc1] | N-(Biphenyl-2-yl)-N- methyl-4-{[(pyridin- 3- ylmethyl)carbamoyl] amino}benzamide | 437.1960 | 57, 1, 33, 61 |
| 564 | 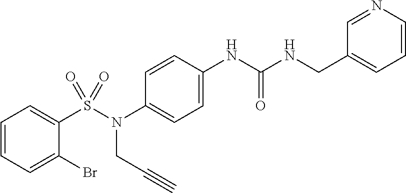 [SMILES: C#CCN(c1ccc(NC(=O)NCc2cccnc2)cc1)S(=O)(=O)c1ccccc1Br] | 2-Bromo-N-(prop-2- yn-1-yl)-N-(4- {[(pyridin-3- ylmethyl)carbamoyl] amino}phenyl) benzenesulfonamide | 499.0415 | 1, 2, 3, 19 |
| 565 | 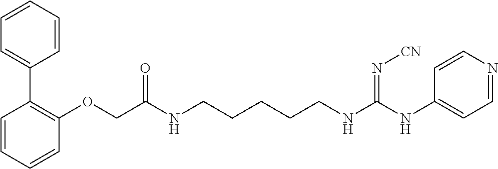 [SMILES: N#C/N=C(/NCCCCCNC(=O)COc1ccccc1-c1ccccc1)Nc1ccncc1] | N-(2-Bromophenyl)- 4-{[(pyridin-4- ylmethyl)carbamoyl] amino} benzenesulfonamide | | 5, 56, 18 |
| 566 | 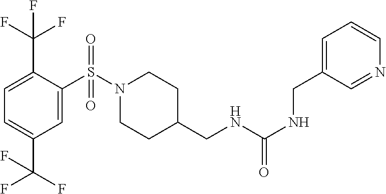 [SMILES: O=C(NCc1cccnc1)NCC1CCN(S(=O)(=O)c2cc(C(F)(F)F)ccc2C(F)(F)F)CC1] | 1-[(1-{[2,5- Bis(trifluoromethyl) phenyl]sulfonyl} piperidin-4-yl) methyl]-3- (pyridin-3- ylmethyl)urea | 525.1357 | 18, 32, 18 |
| 567 | 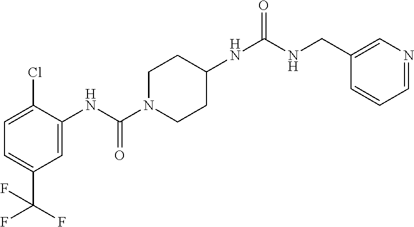 [SMILES: O=C(NCc1cccnc1)NC1CCN(C(=O)Nc2cc(C(F)(F)F)ccc2Cl)CC1] | N-[2-Chloro-5- (trifluoromethyl) phenyl]-4-{[(pyridin-3- ylmethyl)carbamoyl] amino}piperidine-1- carboxamide | 456.1295 | 18, 32, 18 |
| 568 | 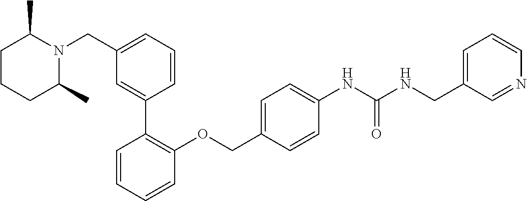 [SMILES: C[C@@H]1CCC[C@H](C)N1Cc1cccc(-c2ccccc2OCc2ccc(NC(=O)NCc3cccnc3)cc2)c1] | 1-(4-{[(3′-{[(2R,6S)- 2,6- Dimethylpiperidin-1- yl]methyl}biphenyl- 2- yl)oxy]methyl}phenyl)- 3-(pyridin-3- ylmethyl)urea | 535.3075 | 44, 45, 46, 4, 48 |
| 569 | 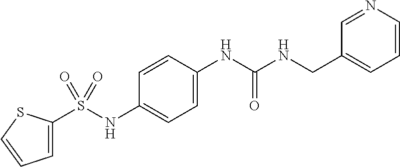 [SMILES: O=C(NCc1cccnc1)Nc1ccc(NS(=O)(=O)c2cccs2)cc1] | N-(4-{[(pyridin-3- ylmethyl)carbamoyl] amino}phenyl) thiophene- 2-sulfonamide | 389.0756 | 1, 2, 3 |
| 570 | 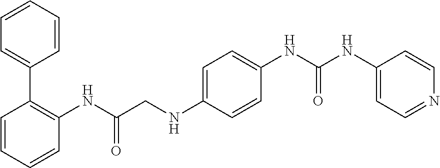 [SMILES: O=C(CNc1ccc(NC(=O)Nc2ccncc2)cc1)Nc1ccccc1-c1ccccc1] | N-Biphenyl-2-yl- N~2~-{4-[(pyridin-4- ylcarbamoyl)amino] phenyl}glycinamide | 438.1868 | 79, 15, 40. 32, 18 |
| 571 | 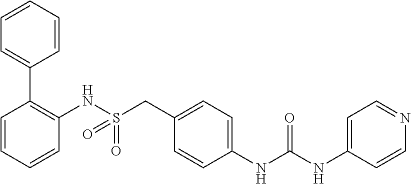 [SMILES: O=C(Nc1ccncc1)Nc1ccc(CS(=O)(=O)Nc2ccccc2-c2ccccc2)cc1] | N-(Biphenyl-2-yl)-1- {4-[(pyridin-4- ylcarbamoyl)amino] phenyl} methanesulfonamide | 459.1504 | 3, 32, 18 |
| 572 | 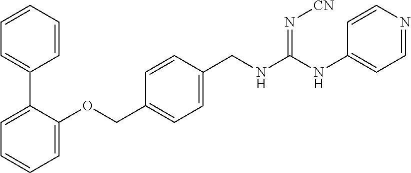 [SMILES: N#C/N=C(/NCc1ccc(COc2ccccc2-c2ccccc2)cc1)Nc1ccncc1] | N-(4-{[(1H- benzimidazol-2- ylmethyl)carbamoyl] amino}phenyl)biphenyl- 2-sulfonamide | 498.1573 | 17, 18 |
| 573 | 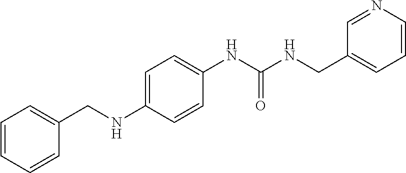 [SMILES: O=C(NCc1cccnc1)Nc1ccc(NCc2ccccc2)cc1] | 1-[4- (Benzylamino)phenyl]- 3-(pyridin-3- ylmethyl)urea | 333.1716 | 1, 2, 35 |
| 574 | 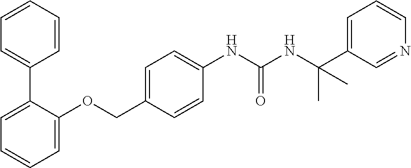 [SMILES: CC(C)(NC(=O)Nc1ccc(COc2ccccc2-c2ccccc2)cc1)c1cccnc1] | 1-{4-[(Biphenyl-2- yloxy)methyl]phenyl}- 3-[2-(pyridin-3- yl)propan-2-yl]urea | 438.2174 | 37, 32, 18 |
| 575 | 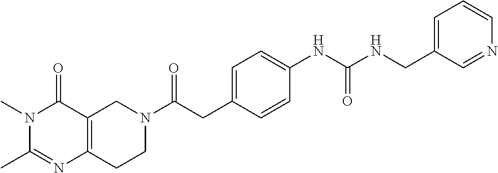 [SMILES: Cc1nc2c(c(=O)n1C)CN(C(=O)Cc1ccc(NC(=O)NCc3cccnc3)cc1)CC2] | 1-{4-[2-(2,3- Dimethyl-4-oxo- 3,5,7,8- tetrahydropyrido[4,3- d]pyrimidin-6(4H)- yl)-2- oxoethyl]phenyl}-3- (pyridin-3- ylmethyl)urea | 447.2143 | 30, 31, 32, 33, 34 |
| 576 | 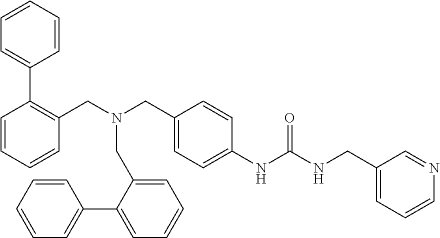 [SMILES: O=C(NCc1cccnc1)Nc1ccc(CN(Cc2ccccc2-c2ccccc2)Cc2ccccc2-c2ccccc2)cc1] | 1-(4-{[Bis(biphenyl- 2- ylmethyl)amino] methyl}phenyl)-3- (pyridin-3- ylmethyl)urea | 589.2962 | 18, 32, 35 |
| 577 | 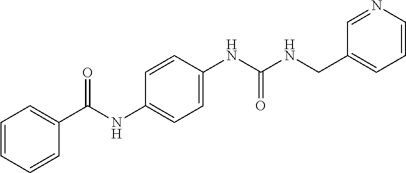 [SMILES: O=C(NCc1cccnc1)Nc1ccc(NC(=O)c2ccccc2)cc1] | N-(4-{[(Pyridin-3- ylmethyl)carbamoyl] amino}phenyl) benzamide | 347.1493 | 1, 2, 9 |
| 578 | 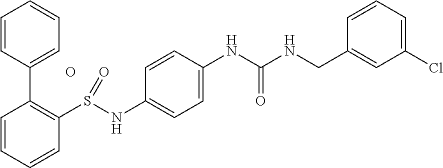 | N-(4-{[(3- chlorobenzyl)carbamoyl] amino}phenyl) biphenyl-2- sulfonamide | 492.1135 | 17, 18 |
| 579 | 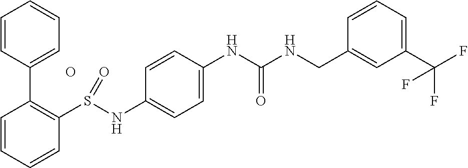 | N-[4-({[3- (trifluoromethyl) benzyl]carbamoyl} amino) phenyl]biphenyl-2- sulfonamide | 526.1418 | 17, 18 |
| 580 | 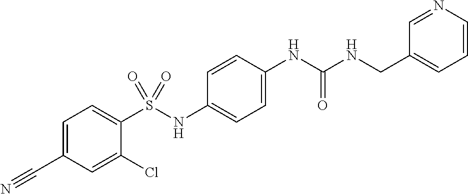 [SMILES: N#Cc1ccc(S(=O)(=O)Nc2ccc(NC(=O)NCc3cccnc3)cc2)c(Cl)c1] | 2-Chloro-4-cyano-N- (4-{[(pyridin-3- ylmethyl)carbamoyl] amino}phenyl) benzenesulfonamide | 442.0743 | 1, 2, 3 |
| 581 | 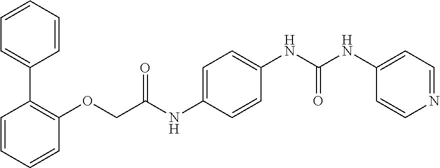 [SMILES: O=C(COc1ccccc1-c1ccccc1)Nc1ccc(NC(=O)Nc2ccncc2)cc1] | 2-(Biphenyl-2- yloxy)-N-{4- [(pyridin-4- ylcarbamoyl)amino] phenyl}acetamide | 439.1776 | 10, 15, 40, 18 |
| 582 | 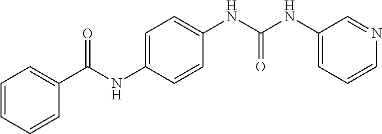 [SMILES: O=C(Nc1ccc(NC(=O)c2ccccc2)cc1)Nc1cccnc1] | N-{4-[(Pyridin-3- ylcarbamoyl)amino] phenyl}benzamide | 333.1441 | 1, 2, 40 |
| 583 | 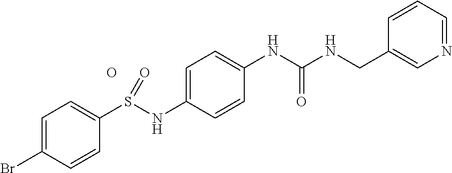 | 4-Bromo-N-(4- {[(pyridin-3- ylmethyl)carbamoyl] amino}phenyl) benzenesulfonamide | 461.0240 | 18, 3 |
| 584 | 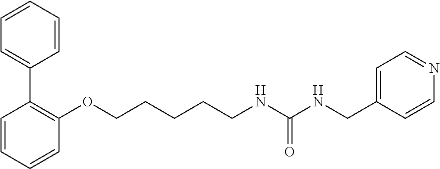 [SMILES: O=C(NCCCCCOc1ccccc1-c1ccccc1)NCc1ccncc1] | 1-[5-(Biphenyl-2- yloxy)pentyl]-3- (pyridin-4- ylmethyl)urea | 390.2226 | 10, 18 |
| 585 | 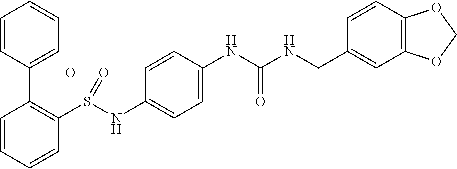 | N-(4-{[(1,3- benzodioxol-5- ylmethyl)carbamoyl] amino}phenyl)biphenyl- 2-sulfonamide | 476.1444 | 17, 18 |
| 586 | 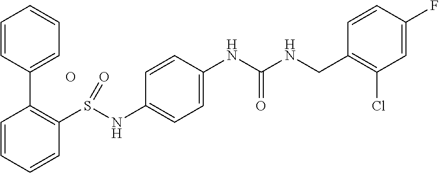 | N-(4-{[(2-chloro-4- fluorobenzyl)carbamoyl] amino}phenyl) biphenyl-2- sulfonamide | 510.1048 | 17, 18 |
| 587 | 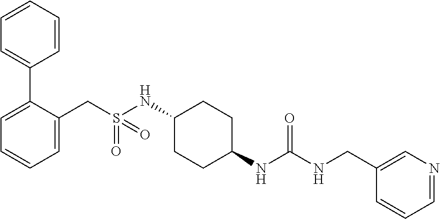 [SMILES: O=C(NCc1cccnc1)N[C@H]1CC[C@H](NS(=O)(=O)Cc2ccccc2-c2ccccc2)CC1] | 1-(Biphenyl-2-yl)-N- (trans-4-{[(pyridin-3- ylmethyl)carbamoyl] amino}cyclohexyl) methanesulfonamide | 479.2117 | 18, 32, 3, 4 |
| 588 | 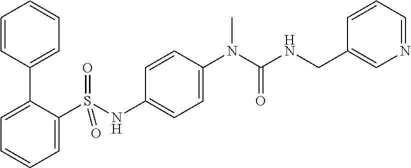 [SMILES: CN(C(=O)NCc1cccnc1)c1ccc(NS(=O)(=O)c2ccccc2-c2ccccc2)cc1] | N-(4- {methyl[(pyridin-3- ylmethyl)carbamoyl] amino}phenyl) biphenyl-2-sulfonamide | 473.1723 | 17, 18 |
| 589 | 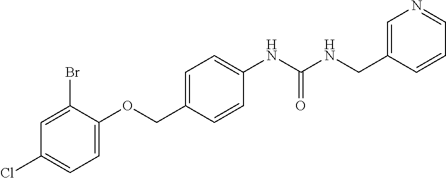 [SMILES: O=C(NCc1cccnc1)Nc1ccc(COc2ccc(Cl)cc2Br)cc1] | 1-{4-[(2-Bromo-4- chlorophenoxy)methyl] phenyl}-3- (pyridin-3- ylmethyl)urea | 446.0271 | 7, 8 |
| 590 | 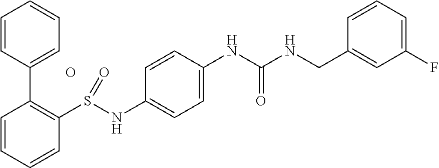 | N-(4-{[(3- fluorobenzyl)carbamoyl] amino}phenyl) biphenyl-2- sulfonamide | 476.1438 | 17, 18 |
| 591 | 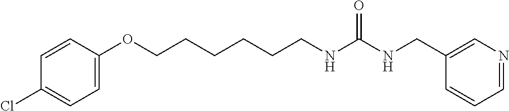 [SMILES: O=C(NCCCCCCOc1ccc(Cl)cc1)NCc1cccnc1] | 1-[6-(4- Chlorophenoxy)hexyl]- 3-(pyridin-3- ylmethyl)urea | 362.1635 | 10, 18 |
| 592 | 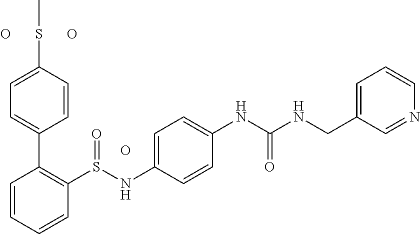 | 4′-(Methylsulfonyl)- N-(4-{[(pyridin-3- ylmethyl)carbamoyl] amino}phenyl)biphenyl- 2-sulfonamide | 537.1325 | 1, 2, 3, 4 |
| 593 | 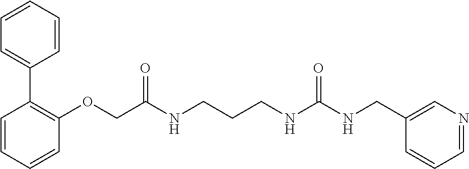 [SMILES: O=C(COc1ccccc1-c1ccccc1)NCCCNC(=O)NCc1cccnc1] | 2-(Biphenyl-2- yloxy)-N-(3- {[(pyridin-3- ylmethyl)carbamoyl] amino}propyl)acetamide | 419.2093 | 10, 15, 40, 18 |
| 594 | 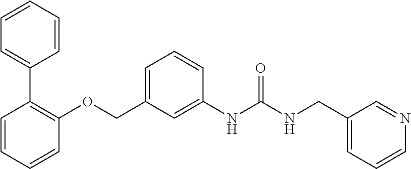 [SMILES: O=C(NCc1cccnc1)Nc1cccc(COc2ccccc2-c2ccccc2)c1] | 1-{3-[(Biphenyl-2- yloxy)methyl]phenyl}- 3-(pyridin-3- ylmethyl)urea | 410.1877 | 74, 10, 18 |

TABLE 3A

| Example | | ¹H NMR Data (400 MHz, |
|---|---|---|
| Number | Structure | DMSO-d6) |
| 595 | 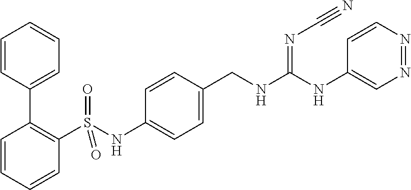 [SMILES: N#C/N=C(/NCc1ccc(NS(=O)(=O)c2ccccc2-c2ccccc2)cc1)Nc1ccnnc1] | 10.10 (s, 1H); 8.81 (s, 1H); 8.52 (s, 1H); 8.00-7.95 (m, 1H); 7.66-7.52 (m, 2H); 7.43-7.33 (m, 4H); 7.31-7.11 (m, 6H); 6.93-6.87 (m, 2H); 4.36 (d, 2H) |
| 596 | 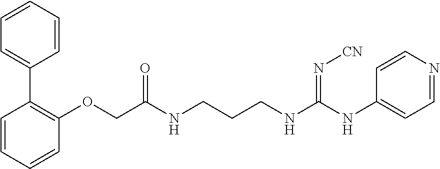 [SMILES: N#C/N=C(/NCCCNC(=O)COc1ccccc1-c1ccccc1)Nc1ccncc1] | 8.57 (d, 2H), 7.88 (t, 1H), 7.60 (d, 3H), 7.43 (t, 3H), 7.37-7.30 (m, 3H), 7.08 (td, 1H), 7.01 (d, 1H), 4.50 (s, 2H), 3.34-3.26 (m, 2H), 3.22-3.12 (m, 2H), 1.76-1.64 (m, 2H) |
| 597 | 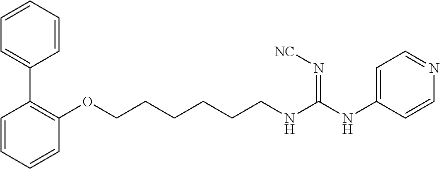 [SMILES: N#C/N=C(\NCCCCCCOc1ccccc1-c1ccccc1)Nc1ccncc1] | 8.50 (d, 2H), 8.41 (bs, 1H, 7.49 (dd, 2H), 7.40 (t, 3H), 7.34-7.27 (m, 3H), 7.10 (d, 1H), 7.02 (td, 1H), 3.98 (5, 2H), 3.32-3.23 (m, 2H), 1.69-1.61 (m, 2H), 1.56-1.47 (m, 2H), 1.41- 1.26 (m, 4H) |
| 598 | 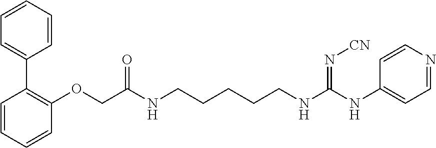 [SMILES: N#C/N=C(/NCCCCCNC(=O)COc1ccccc1-c1ccccc1)Nc1ccncc1] | 8.57 (d, 2H), 7.67 (t, 1H), 7.59 (dd, 2H), 7.43 (t, 2H), 7.37-7.30 (m, 3H), 7.07 (t, 1H), 7.00 (dd, 1H), 4.48 (s, 2H), 3.33-3.25 (m, 2H), 3.14-3.06 (m, 2H), 1.58-1.49 (m, 2H), 1.46- 1.36 (m, 2H), 1.29-1.20 (m, 2H) |
| 599 | 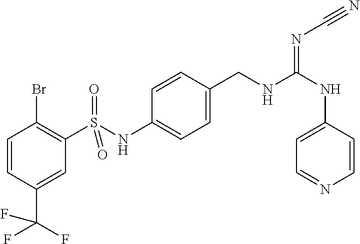 [SMILES: N#C/N=C(/NCc1ccc(NS(=O)(=O)c2cc(C(F)(F)F)ccc2Br)cc1)Nc1ccncc1] | 10.96 (s, 1H); 9.00 (s, 1H); 8.55 (d, 2H); 8.24 (s, 1H); 8.09 (d, 1H); 7.95-7.89 (m, 1H); 7.46 (s, 2H); 7.24 (d, 2H); 7.10 (d, 2H); 4.42 (d, 2H) |
| 600 | 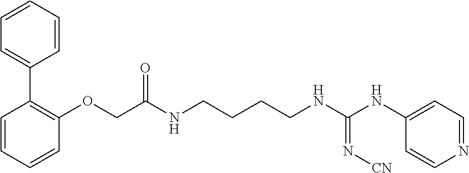 [SMILES: N#C/N=C(/NCCCCNC(=O)COc1ccccc1-c1ccccc1)Nc1ccncc1] | n/a |
| 601 | 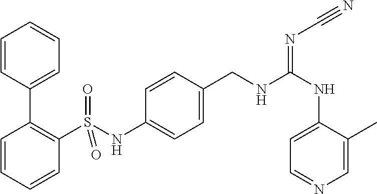 [SMILES: Cc1cnccc1N/C(=N\C#N)NCc1ccc(NS(=O)(=O)c2ccccc2-c2ccccc2)cc1] | 10.14 (s, 1H); 8.98 (s, 1H); 8.54 (s, 1H); 8.49 (d, 1H); 7.99 (d, 1H); 7.67-7.54 (m, 2H); 7.44- 7.35 (m, 4H); 7.33-7.28 (m, 1H); 7.28-7.23 (m, 2H); 7.20 (d, 2H); 6.93 (d, 2H); 4.42 (d, 2H); 2.21 (s, 3H) |
| 602 | 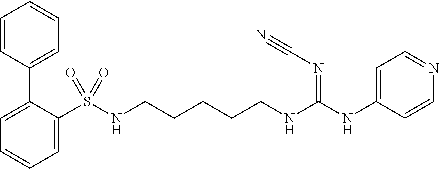 [SMILES: N#C/N=C(\NCCCCCNS(=O)(=O)c1ccccc1-c1ccccc1)Nc1ccncc1] | 8.58 (d, 2H); 7.93 (dd, 1H); 7.65 (dt, 1H); 7.59 (dt, 1H); 7.39 (m, 6H), 7.26 (t, 1H); 3.27 (q, 2H); 2.66 (q, 2H); 1.47 (p, 2H); 1.35 (p, 2H), 1.25 (m, 2H) |
| 603 | 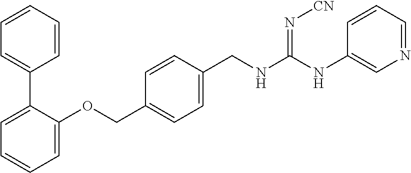 [SMILES: N#C/N=C(/NCc1ccc(COc2ccccc2-c2ccccc2)cc1)Nc1cccnc1] | 9.34 (s, 1H), 8.51 (d, 1H), 8.39 (dd, 1H), 7.99 (bs, 1H), 7.75 (d, 1H), 7.53 (d, 2H), 7.49-7.25 (m, 12H), 7.18 (d, 1H), 7.04 (t, 1H), 5.12 (s, 2H), 4.43 (d, 2H) |
| 604 | 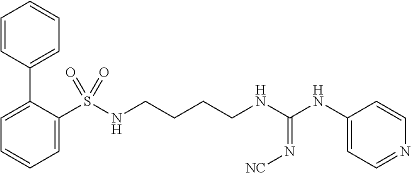 [SMILES: N#C/N=C(\NCCCCNS(=O)(=O)c1ccccc1-c1ccccc1)Nc1ccncc1] | 8.39 (d, 2H), 7.93 (dd, 1H), 7.85 (bs, 1H), 7.65 (td, 1H), 7.59 (td, 1H), 7.41-7.29 (m, 6H), 7.21 (bs, 1H), 3.22-3.15 (m, 2H), 2.71-2.64 (m, 2H), 1.49-1.40 (m, 2H), 1.40-1.31 (m, 2H) |
| 605 | 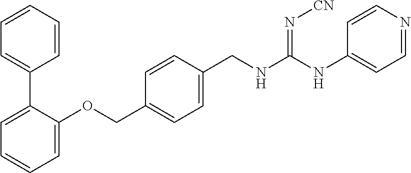 [SMILES: N#C/N=C(/NCc1ccc(COc2ccccc2-c2ccccc2)cc1)Nc1ccncc1] | 8.55 (d, 2H), 7.52 (dd, 2H), 7.43- 7.29 (m, 10H), 7.18 (d, 1H), 7.05 (t, 1H), 5.13 (s, 2H), 4.53 (d, 2H) |
| 606 | 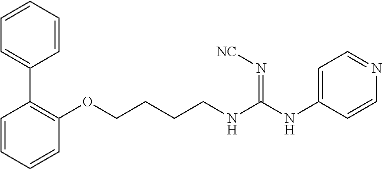 [SMILES: N#C/N=C(\NCCCCOc1ccccc1-c1ccccc1)Nc1ccncc1] | 8.44 (d, 2H), 8.21 (bs, 1H), 7.49 (d, 2H), 7.38 (t, 2H), 7.35-7.25 (m, 5H), 7.11 (d, 1H), 7.03 (t, 1H), 4.02 (t, 2H), 3.33-3.26 (m, 2H), 3.14-3.06 (m, 2H), 1.73-1.59 (m, 4H) |
| 607 | 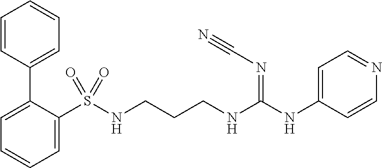 [SMILES: N#C/N=C(\NCCCNS(=O)(=O)c1ccccc1-c1ccccc1)Nc1ccncc1] | 8.39 (d, 2H), 7.93 (dd, 1H), 7.85 (bs, 1H), 7.65 (td, 1H), 7.59 (td, 1H), 7.41-7.29 (m, 6H), 7.21 (bs, 1H), 3.22-3.15 (m, 2H), 2.71-2.64 (m, 2H), 1.49-1.40 (m, 2H), 1.40-1.31 (m, 2H) |
| 608 | 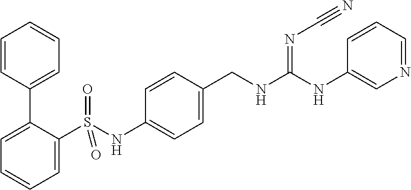 [SMILES: N#C/N=C(/NCc1ccc(NS(=O)(=O)c2ccccc2-c2ccccc2)cc1)Nc1cccnc1] | 10.08 (s, 1H); 9.38 (s, 1H); 8.57- 8.53 (m, 1H); 8.44-8.41 (m, 1H); 8.01-7.93 (m, 1H); 7.85-7.78 (m, 1H); 7.66-7.49 (m, 3H); 7.42-7.36 (m, 3H); 7.31-7.26 (m, 1H); 7.25-7.20 (m, 2H); 7.14 (d, 2H); 6.91 (d, 2H); 4.33 (d, 2H) |
| 609 | 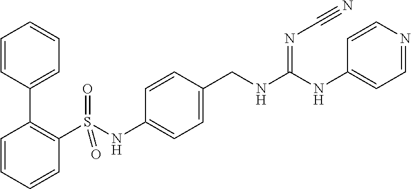 [SMILES: N#C/N=C(/NCc1ccc(NS(=O)(=O)c2ccccc2-c2ccccc2)cc1)Nc1ccncc1] | 10.08 (s, 1H); 8.45-8.32 (m, 3H); 8.29 (t, 1H); 7.98 (d, 1H); 7.63 (t, 1H); 7.55 (t, 1H); 7.45 (d, 1H); 7.42- 7.33 (m, 3H); 7.28 (d, 1H); 7.26- 7.20 (m, 2H); 7.20-7.12 (m, 3H); 6.91 (d, 2H); 4.36 (d, 2H) |
| 610 | 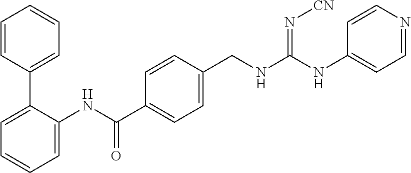 [SMILES: N#C/N=C(/NCc1ccc(C(=O)Nc2ccccc2-c2ccccc2)cc1)Nc1ccncc1] | 10.96 (s, 1H); 9.00 (s, 1H); 8.55 (d, 2H); 8.24 (s, 1H); 8.09 (d, 1H); 7.95-7.89 (m, 1H); 7.46 (s, 2H); 7.24 (d, 2H); 7.10 (d, 2H); 4.42 (d, 2H) |
| 611 | 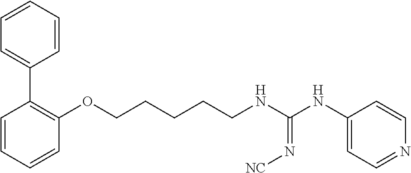 [SMILES: N#C/N=C(\NCCCCCOc1ccccc1-c1ccccc1)Nc1ccncc1] | 8.47 (d, 2H), 8.28 (bs, 1H), 7.49 (d, 2H). 7.40 (t, 2H), 7.34-7.26 (m, 4H), 7.10 (d, 1H), 7.02 (t, 1H), 3.99 (t, 2H), 3.33-3.23 (m, 2H), 1.71-1.64 (m, 2H), 1.59-1.51 (m, 2H), 1.43- 1.34 (m, 2H) |
| 612 | 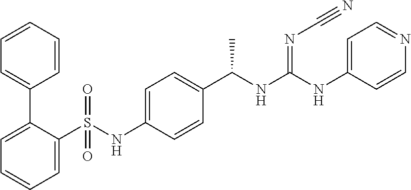 [SMILES: C[C@H](N/C(=N/C#N)Nc1ccncc1)c1ccc(NS(=O)(=O)c2ccccc2-c2ccccc2)cc1] | n/a |

TABLE 3B

| | | HRMS | |
|---|---|---|---|
| Example | | Found | Synthetic |
| Number | IUPAC Name | [M + H]⁺ | Procedures |
| 595 | N-[4-({[(Z)-(Cyanoamino)(pyridazin-4- | 484.1494 | 17, 87 |
| | ylamino)methylidene]amino}methyl)phenyl]biphenyl- | | |
| | 2-sulfonamide | | |
| 596 | 2-(Biphenyl-2-yloxy)-N-(3-{[(Z)- | 429.2043 | 14, 15, 16, 32, |
| | (cyanoamino)(pyridin-4- | | 87 |
| | ylamino)methylidene]amino}propyl)acetamide | | |
| 597 | 1-[6-(Biphenyl-2-yloxy)hexyl]-2-cyano-3-pyridin-4- | 424.2285 | 10, 87 |
| | ylguanidine | | |
| 598 | 2-(Biphenyl-2-yloxy)-N-(5-{[(Z)- | n/a | 14, 15, 16, 32, |
| | (cyanoamino)(pyridin-4- | | 87 |
| | ylamino)methylidene]amino}pentyl)acetamide | | |
| 599 | 2-Bromo-N-[4-({[(Z)-(cyanoamino)(pyridin-4- | 555.0149 | 17, 87 |
| | ylamino)methylidene]amino}methyl)phenyl]-5- | | |
| | (trifluoromethyl)benzenesulfonamide | | |
| 600 | 2-(Biphenyl-2-yloxy)-N-(4-{[(Z)- | 443.2204 | 14, 15, 1 6, 32, |
| | (cyanoamino)(pyridin-4- | | 87 |
| | ylamino)methylidene]amino} butyl)acetamide | | |
| 601 | N-{4-[({(Z)-(Cyanoamino) [(3-methylpyridin-4- | 497.1889 | 17, 87 |
| | yl)amino]methylidene}amino)methyl]phenyl}biphenyl- | | |
| | 2-sulfonamide | | |
| 602 | N-(5-{[(Z)-(Cyanoamino)(pyridin-4- | 463.1926 | 17, 87 |
| | ylamino)methylidene]amino}pentyl)biphenyl-2- | | |
| | sulfonamide | | |
| 603 | 1-{4-[(Biphenyl-2-yloxy)methyl]benzyl}-2-cyano -3- | 434.2002 | 10, 87 |
| | pyridin-3-ylguanidine | | |
| 604 | N-(4-{[(Z)-(Cyanoamino)(pyridin-4- | 449.1755 | 17, 87 |
| | ylamino)methylidene]amino}butyl)biphenyl-2- | | |
| | sulfonamide | | |
| 605 | 1-{4-[(Biphenyl-2-yloxy)methyl]benzyl}-2-cyano-3- | 434.1921 | 10, 87 |
| | pyridin-4-ylguanidine | | |
| 606 | 1-[4-(Biphenyl-2-yloxy)butyl]-2-cyano-3-pyridin-4- | 386.1972 | 10, 87 |
| | ylguanidine | | |
| 607 | N-(3-{[(Z)-(Cyanoamino)(pyridin-4- | 435.1623 | 17, 87 |
| | ylamino)methylidene]amino}propyl)biphenyl-2- | | |
| | sulfonamide | | |
| 608 | N-[4-({[(Z)-(Cyanoamino)(pyridin-3- | 483.1582 | 17, 87 |
| | ylamino)methylidene]amino}methyl)phenyl]biphenyl- | | |
| | 2-sulfonamide | | |
| 609 | N-[4-({[(Z)-(Cyanoamino)(pyridin-4- | 483.1541 | 17, 87 |
| | ylamino)methylidene]amino}methyl)phenyl]biphenyl- | | |
| | 2-sulfonamide | | |
| 610 | N-(Biphenyl-2-yl)-4-({[(Z)-(cyanoamino)(pyridin-4- | 447.1915 | 9, 32, 87 |
| | ylamino)methylidene]amino}methyl)benzamide | | |
| 611 | 1-[5-(Biphenyl-2-yloxy)pentyl]-2-cyano-3-pyridin-4- | 400.2125 | 10, 87 |
| | ylguanidine | | |
| 612 | N-{4-[(1S)-1-{[(Z)-(Cyanoamino)(pyridin-4- | 497.1862 | 17, 87 |
| | ylamino)methylidene]amino}ethyl]phenyl}biphenyl-2- | | |
| | sulfonamide | | |

TABLE 4

| | | | HRMS | |
|---|---|---|---|---|
| | | | Found | Syn. |
| Ex. No. | Structure | IUPAC Name | [M + H]⁺ | Proc. |
| 613 | 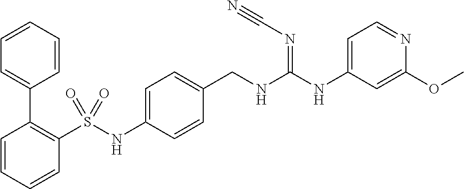 [SMILES: COc1cc(N/C(=N/C#N)NCc2ccc(NS(=O)(=O)c3ccccc3-c3ccccc3)cc2)ccn1] | N-{4-[({(Z)- (Cyanoamino)[(2- methoxypyridin-4- yl)amino]methylidene} amino)methyl]phenyl} biphenyl-2- sulfonamide | 513.1704 | 17, 87 |
| 614 | 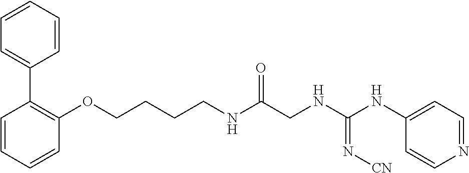 [SMILES: N#C/N=C(/NCC(=O)NCCCCOc1ccccc1-c1ccccc1)Nc1ccncc1] | (Z)-N-[4-(Biphenyl-2- yloxy)butyl]-N~2~- [(cyanoamino)(pyridin- 4- ylamino)methylidene] glycinamide | 443.2188 | 10, 9, 32, 87 |
| 615 | 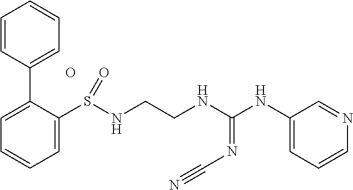 | N-(2-{[(Z)- (Cyanoamino)(pyridin- 3- ylamino)methylidene] amino}ethyl)biphenyl- 2-sulfonamide | 421.1432 | 17, 87 |
| 616 | 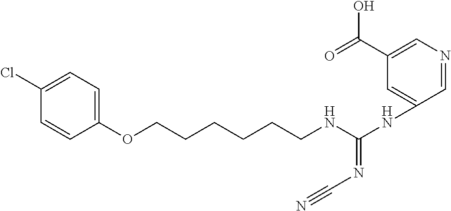 [SMILES: N#C/N=C(\NCCCCCCOc1ccc(Cl)cc1)Nc1cncc(C(=O)O)c1] | 5-{[{[6-(4- Chlorophenoxy)hexyl] amino}(cyanoamino) methylidene]amino}pyri- dine-3-carboxylic acid | 416.1478 | 41, 42, 87 |
| 617 | 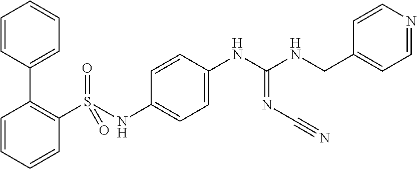 [SMILES: N#C/N=C(\NCc1ccncc1)Nc1ccc(NS(=O)(=O)c2ccccc2-c2ccccc2)cc1] | N-[4-({(Z)- (Cyanoamino)[(pyridin- 4- ylmethyl)amino]methyl- idene}amino)phenyl] biphenyl-2-sulfonamide | 483.1651 | 17, 87 |
| 618 | 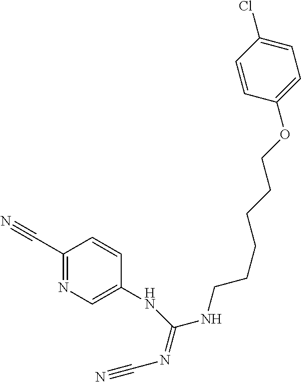 [SMILES: N#C/N=C(/NCCCCCCOc1ccc(Cl)cc1)Nc1ccc(C#N)nc1] | 1-[6-(4- Chlorophenoxy)hexyl]- 2-cyano-3-(6- cyanopyridin-3- yl)guanidine | NA | 41, 42, 87 |
| 619 | 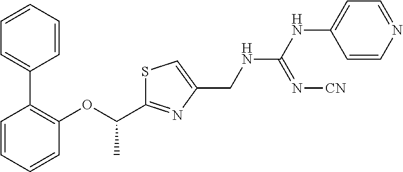 [SMILES: C[C@H](Oc1ccccc1-c1ccccc1)c1nc(CN/C(=N/C#N)Nc2ccncc2)cs1] | 1-({2-[(1S)-1- (Biphenyl-2- yloxy)ethyl]-1,3- thiazol-4-yl}methyl)- 2-cyano-3-pyridin-4- ylguanidine | 455.1645 | 14, 15, 24, 25, 26, 87 |
| 620 | 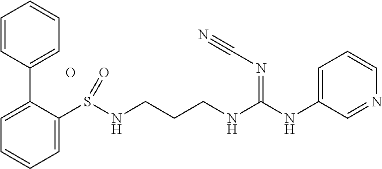 | N-(3-{[(Z)- (Cyanoamino)(pyridin- 3- ylamino)methylidene] amino}propyl)biphenyl- 2-sulfonamide | 435.1589 | 17, 87 |
| 621 | 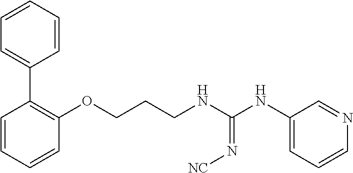 [SMILES: N#C/N=C(\NCCCOc1ccccc1-c1ccccc1)Nc1cccnc1] | 1-[3-(Biphenyl-2- yloxy)propyl]-2- cyano-3-pyridin-3- ylguanidine | 372.1820 | 10, 87 |
| 622 | 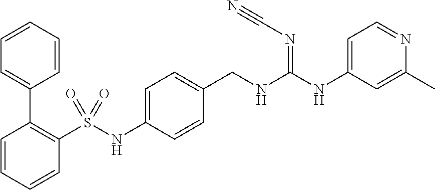 [SMILES: Cc1cc(N/C(=N/C#N)NCc2ccc(NS(=O)(=O)c3ccccc3-c3ccccc3)cc2)ccn1] | N-{4-[({(Z)- (Cyanoamino)[(2- methylpyridin-4- yl)amino]methylidene} amino)methyl]phenyl} biphenyl-2- sulfonamide | 497.1764 | 17, 87 |
| 623 | 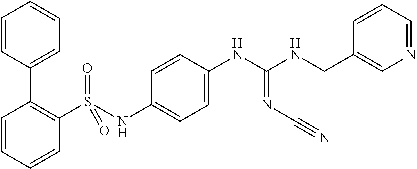 [SMILES: N#C/N=C(\NCc1cccnc1)Nc1ccc(NS(=O)(=O)c2ccccc2-c2ccccc2)cc1] | N-[4-({(Z)- (Cyanoamino)[(pyridin- 3- ylmethyl)amino]methyl- idene}amino)phenyl] biphenyl-2-sulfonamide | 483.1662 | 17, 87 |
| 624 | 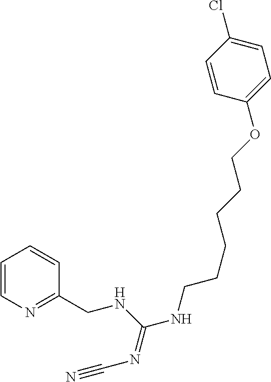 [SMILES: N#C/N=C(/NCCCCCCOc1ccc(Cl)cc1)NCc1ccccn1] | 1-[6-(4- Chlorophenoxy)hexyl]- 2-cyano-3-(pyridin-2- ylmethyl)guanidine | 386.1748 | 41, 42, 87 |
| 625 | 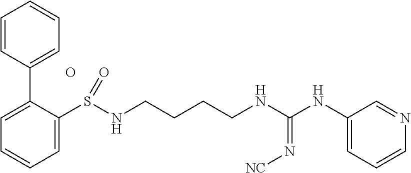 | N-(4-{[(Z)- (Cyanoamino)(pyridin- 3- ylamino)methylidene] amino}butyl)biphenyl- 2-sulfonamide | 449.1747 | 17, 87 |
| 626 | 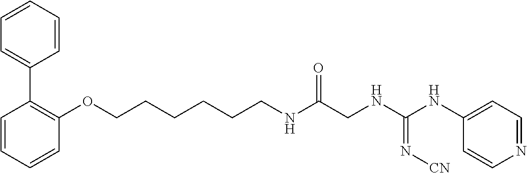 [SMILES: N#C/N=C(/NCC(=O)NCCCCCCOc1ccccc1-c1ccccc1)Nc1ccncc1] | (Z)-N-[6-(Biphenyl-2- yloxy)hexyl]-N~2~- [(cyanoamino)(pyridin- 4- ylamino)methylidene] glycinamide | 471.2508 | 10, 9, 32, 87 |
| 627 | 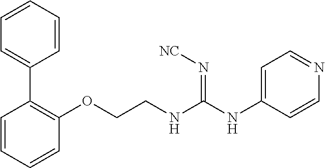 [SMILES: N#C/N=C(\NCCOc1ccccc1-c1ccccc1)Nc1ccncc1] | 1-[2-(Biphenyl-2- yloxy)ethyl]-2-cyano- 3-pyridin-4- ylguanidine | 358.1653 | 10, 87 |
| 628 | 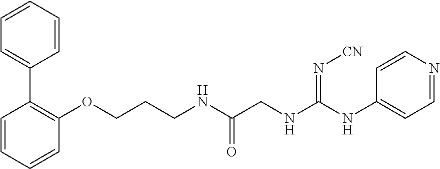 [SMILES: N#C/N=C(/NCC(=O)NCCCOc1ccccc1-c1ccccc1)Nc1ccncc1] | (Z)-N-[3-(Biphenyl-2- yloxy)propyl]-N~2~- [(cyanoamino)(pyridin- 4- ylamino)methylidene] glycinamide | 429.2045 | 10, 9, 32, 87 |
| 629 | 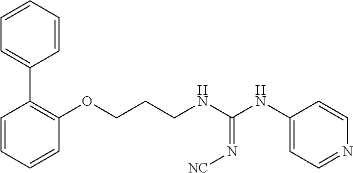 [SMILES: N#C/N=C(\NCCCOc1ccccc1-c1ccccc1)Nc1ccncc1] | 1-[3-(Biphenyl-2- yloxy)propyl]-2- cyano-3-pyridin-4- ylguanidine | 372.1798 | 10, 87 |
| 630 | 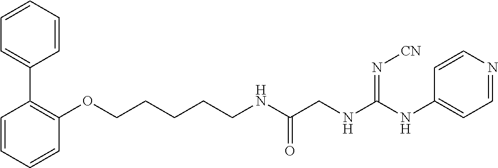 [SMILES: N#C/N=C(/NCC(=O)NCCCCCOc1ccccc1-c1ccccc1)Nc1ccncc1] | (Z)-N-[5-(Biphenyl-2- yloxy)pentyl]-N~2~- [(cyanoamino)(pyridin- 4- ylamino)methylidene] glycinamide | 457.2350 | 10, 9, 32, 87 |
| 631 | 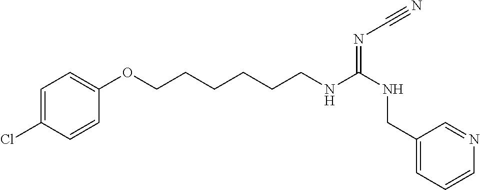 [SMILES: N#C/N=C(/NCCCCCCOc1ccc(Cl)cc1)NCc1cccnc1] | 1-[6-(4- Chlorophenoxy)hexyl]- 2-cyano-3-(pyridin-3- ylmethyl)guanidine | 386.1778 | 41, 42, 87 |
| 632 | 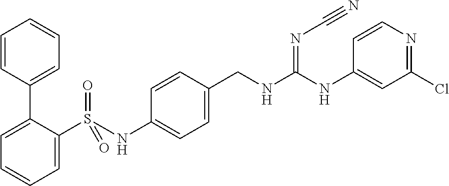 [SMILES: N#C/N=C(/NCc1ccc(NS(=O)(=O)c2ccccc2-c2ccccc2)cc1)Nc1ccnc(Cl)c1] | N-[4-({[(Z)-[(2- Chloropyridin-4- yl)amino](cyanoamino) methylidene]amino} methyl)phenyl]biphenyl- 2-sulfonamide | 517.1114 | 17, 87 |
| 633 | 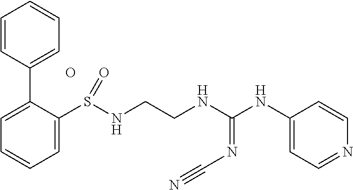 | N-(2-{[(Z)- (Cyanoamino)(pyridin- 4- ylamino)methylidene] amino}ethyl)biphenyl- 2-sulfonamide | 421.1447 | 17, 87 |
| 634 | 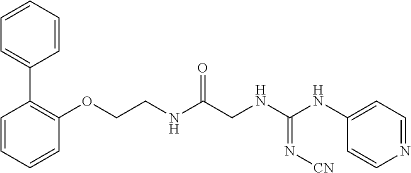 [SMILES: N#C/N=C(/NCC(=O)NCCOc1ccccc1-c1ccccc1)Nc1ccncc1] | (Z)-N-[2-(Biphenyl-2- yloxy)ethyl]-N~2~- [(cyanoamino)(pyridin- 4- ylamino)methylidene] glycinamide | 415.1870 | 10, 9, 32, 87 |
| 635 | 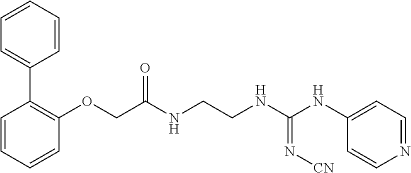 [SMILES: N#C/N=C(/NCCNC(=O)COc1ccccc1-c1ccccc1)Nc1ccncc1] | 2-(Biphenyl-2-yloxy)- N-(2-{[(Z)- (cyanoamino)(pyridin- 4- ylamino)methylidene] amino}ethyl)acetamide | 415.1866 | 14, 15, 16, 32, 87 |
| 636 | 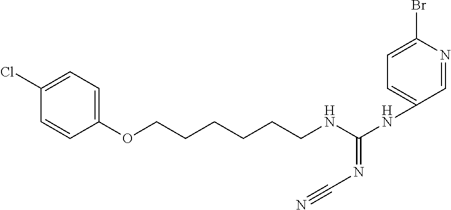 [SMILES: N#C/N=C(\NCCCCCCOc1ccc(Cl)cc1)Nc1ccc(Br)nc1] | 1-(6-Bromopyridin-3- yl)-2-[6-(4- chlorophenoxy)hexyl]- 3-cyanoguanidine | 450.0668 | 41, 42, 87 |
| 637 | 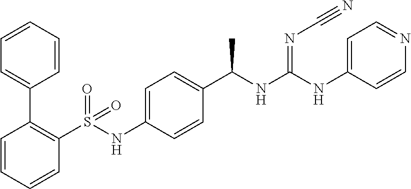 [SMILES: C[C@@H](N/C(=N/C#N)Nc1ccncc1)c1ccc(NS(=O)(=O)c2ccccc2-c2ccccc2)cc1] | N-{4-[(1R)-1-{[(Z)- (Cyanoamino)(pyridin- 4- ylamino)methylidene] amino}ethyl]phenyl}bi- phenyl-2-sulfonamide | 497.1850 | 17, 87 |
| 638 | 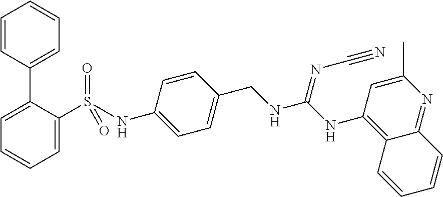 [SMILES: Cc1cc(N/C(=N\C#N)NCc2ccc(NS(=O)(=O)c3ccccc3-c3ccccc3)cc2)c2ccccc2n1] | N-{4-[({(Z)- (Cyanoamino)[(2- methylquinolin-4- yl)amino]methylidene} amino)methyl]phenyl} biphenyl-2- sulfonamide | 547.2038 | 17, 87 |
| 639 | 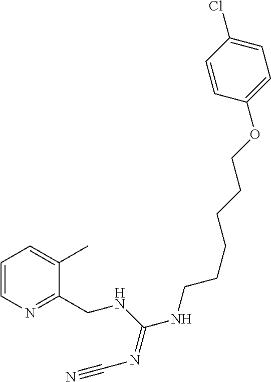 [SMILES: Cc1cccnc1CN/C(=N\C#N)NCCCCCCOc1ccc(Cl)cc1] | 1-[6-(4- Chlorophenoxy)hexyl]- 2-cyano-3-[(3- methylpyridin-2- yl)methyl]guanidine | 400.1904 | 41, 42, 87 |
| 640 | 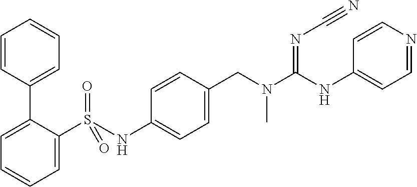 [SMILES: CN(Cc1ccc(NS(=O)(=O)c2ccccc2-c2ccccc2)cc1)/C(=N/C#N)Nc1ccncc1] | N-(4-{[N′-Cyano-N- methyl-N″-(pyridin-4- yl)carbamimidamido] methyl}phenyl)biphenyl- 2-sulfonamide | 497.1773 | 17, 87 |
| 641 | 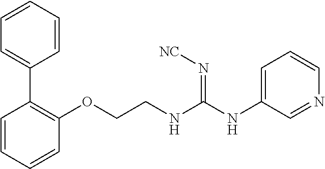 [SMILES: N#C/N=C(\NCCOc1ccccc1-c1ccccc1)Nc1cccnc1] | 1-[2-(Biphenyl-2- yloxy)ethyl]-2-cyano- 3-pyridin-3- ylguanidine | 358.1662 | 10, 87 |
| 642 | 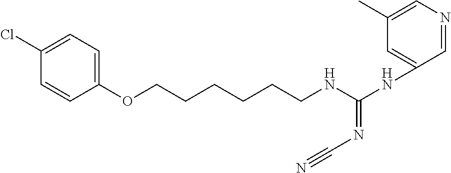 [SMILES: Cc1cncc(N/C(=N/C#N)NCCCCCCOc2ccc(Cl)cc2)c1] | 1-[6-(4- Chlorophenoxy)hexyl]- 2-cyano-3-(5- methylpyridin-3- yl)guanidine | 386.1755 | 41, 42, 87 |
| 643 | 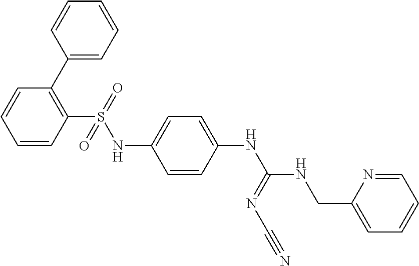 [SMILES: N#C/N=C(\NCc1ccccn1)Nc1ccc(NS(=O)(=O)c2ccccc2-c2ccccc2)cc1] | N-[4-({(Z)- (Cyanoamino)[(pyridin- 2- ylmethyl)amino]methyl- idene}amino)phenyl] biphenyl-2-sulfonamide | 483.1652 | 17, 87 |
| 644 | 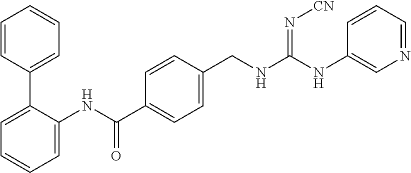 [SMILES: N#C/N=C(/NCc1ccc(C(=O)Nc2ccccc2-c2ccccc2)cc1)Nc1cccnc1] | N-(Biphenyl-2-yl)-4- ({[(Z)- (cyanoamino)(pyridin- 3- ylamino)methylidene] amino}methyl)benzamide | 447.1712 | 9, 32, 87 |

TABLE 5

| No. | Structure | IUPAC Name | LC-MS | NMR |
|---|---|---|---|---|
| 645 | 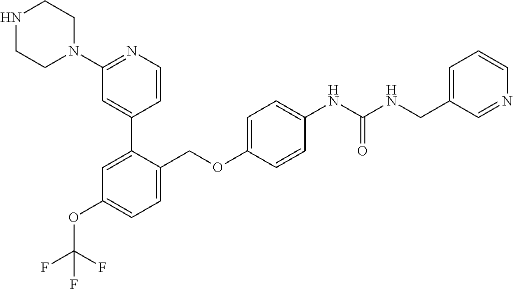 [SMILES: O=C(NCc1cccnc1)Nc1ccc(OCc2ccc(OC(F)(F)F)cc2-c2ccnc(N3CCNCC3)c2)cc1] | 1-[4-({2-[2- (Piperazin-1- yl)pyridin-4-yl]- 4-(trifluoro- methoxy) benzyl}oxy) phenyl]- 3-(pyridin-3- ylmethyl)urea | LC-MS [M + H]+ 579.2329 | |
| 646 | 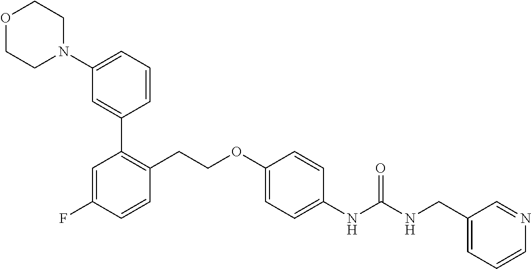 [SMILES: O=C(NCc1cccnc1)Nc1ccc(OCCc2ccc(F)cc2-c2cccc(N3CCOCC3)c2)cc1] | 1-(4-{2-[5- Fluoro- 3′-(morpholin-4- yl)biphenyl-2- yl]ethoxy} phenyl)- 3-(pyridin-3- ylmethyl)urea | LC-MS [M + H]+ 527.2447 | 8.51 (s, 1H), 8.45 (d, 1H), 8.40 (s, 1H), 7.69 (dt, 1H), 7.48 (dd, 1H), 7.39-7.26 (m, 2H), 7.22 (d, 2H), 7.17 (td, 1H), 7.03 (dd, 1H), 6.99 (dd, 1H), 6.90 (s, 1H), 6.79 (d, 1H), 6.64 (d, 2H), 6.59 (t, 1H), 4.29 (d, 2H), 3.96 (t, 2H), 3.74-3.67 (m, 4H), 3.17- 3.10 (m, 4H), 2.94 (t, 2H) |
| 647 | 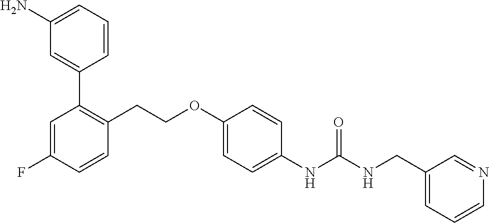 [SMILES: Nc1cccc(-c2cc(F)ccc2CCOc2ccc(NC(=O)NCc3cccnc3)cc2)c1] | 1-{4-[2-(3′- Amino- 5-fluorobiphenyl- 2- yl)ethoxy] phenyl}- 3-(pyridin-3- ylmethyl)urea | LC-MS [M + H]+ 457.2065 | 8.53-8.42 (m, 3H), 8.38 (s, 1H), 7.69 (d, 1H), 7.64 (d, 1H), 7.45 (dd, 1H), 7.39-7.32 (m, 2H), 7.22 (d, 2H), 7.14 (dt , 1H), 7.09 1H), 6.96 (dd, 1H), 6.67 (d, 2H), 6.62-6.55 (m, 2H), 6.51 (s, 1H), 6.46 (d, 1H), 5.21 (s, 2H), 4.29 (d, 2H), 3.93 (t, 2H), 2.94 (t, 2H) |
| 648 | 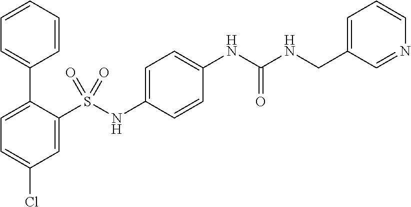 [SMILES: O=C(NCc1cccnc1)Nc1ccc(NS(=O)(=O)c2cc(Cl)ccc2-c2ccccc2)cc1] | 4-Chloro-N-(4- {[(pyridin-3- ylmethyl) carbamoyl] amino}phenyl)bi- phenyl-2- sulfonamide | LC-MS [M + H]+ 493.1078 | |
| 649 | 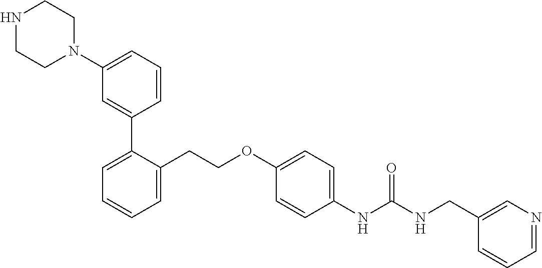 [SMILES: O=C(NCc1cccnc1)Nc1ccc(OCCc2ccccc2-c2cccc(N3CCNCC3)c2)cc1] | 1-(4-{2-[3′- (Piperazin-1- yl)biphenyl-2- yl]ethoxy} phenyl)- 3-(pyridin-3- ylmethyl)urea | LC-MS [M + H]+ 508.2698 | 8.51 (s, 1H), 8.45 (d, 1H), 8.40 (s, 1H), 7.70 (dd, 1H), 7.45 (d, 1H), 7.39-7.26 (m, 4H), 7.25-7.17 (m, 3H), 7.00 (d, 1H), 6.95 (s, 1H), 6.83 (d, 1H) 6.63 (d, 2H), 4.29 (d, 2H), 3.97 (t, 2H), 3.19-3.09 (m, 4H), 2.96 (t, 2H), 2.48- 2.41 (m, 2H) |
| 650 | 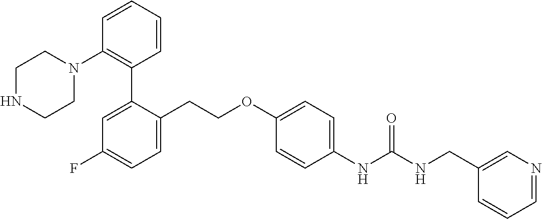 [SMILES: O=C(NCc1cccnc1)Nc1ccc(OCCc2ccc(F)cc2-c2ccccc2N2CCNCC2)cc1] | 1-(4-{2-[5- Fluoro- 2′-(piperazin-1- yl)biphenyl-2- yl]ethoxy} phenyl)- 3-(pyridin-3- ylmethyl)urea | LC-MS [M + H]+ 526.2635 | 8.51 (s, 1H), 8.47 (s, 1H), 8.45 (d, 1H), 7.69 (dt, 1H), 7.54-7.33 (m, 3H), 7.25-7.15 (m, 7H), 7.10 (dd, 1H), 6.65 (t, 1H), 6.59 (d, 2H), 4.29 (d, 2H), 3.96-3.81 (m, 2H), 3.02- 2.74 (m, 10H) |
| 651 | 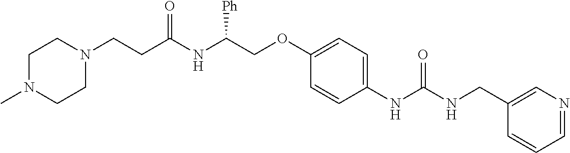 [SMILES: CN1CCN(CCC(=O)N[C@@H](COc2ccc(NC(=O)NCc3cccnc3)cc2)c2ccccc2)CC1] | 3-(4- Methylpiperazin- 1-yl)-N-[(1R)-1- phenyl-2-(4- {[(pyridin-3- ylmethyl) carbamoyl] amino}phenoxy) ethyl] propanamide | LC-MS [M + H]+ 517.2951 | 8.75 (d, 1H), 8.51 (s, 1H), 8.45 (s, 1H), 8.44 (dd, 1H), 7.69 (d, 1H), 7.43-7.24 (m, 7H), 6.82 (d, 2H), 6.63 (t, 1H), 5.18 (q, 1H), 4.29 (d, 2H), 4.07 (t, 2H), 2.97 (q, 1H), 2.43-2.20 (m, 8H), 2.11 (s, 3H) |
| 652 | 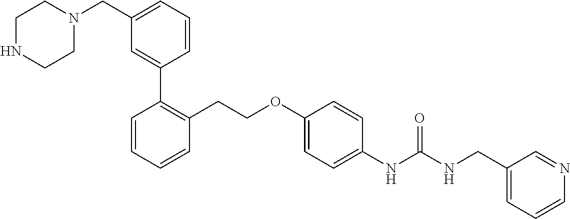 [SMILES: O=C(NCc1cccnc1)Nc1ccc(OCCc2ccccc2-c2cccc(CN3CCNCC3)c2)cc1] | 1-(4-{2-[3′- (Piperazin-1- ylmethyl) biphenyl-2- yl]ethoxy} phenyl)- 3-(pyridin-3- ylmethyl)urea | LC-MS [M + H]+ 522.2886 | 8.65 (s, 1H), 8.62 (d, 1H), 8.54 (s, 1H), 8.05 (d, 1H), 7.67 (dd, 1H), 7.52-7.29 (m, 7H), 7.25-7.20 (m, 3H), 6.73 (t, 1H), 6.67 (d, 2H), 4.36 (d, 2H), 4.01-3.87 (m, 4H), 3.20- 3.10 (m, 4H), 2.97 (t, 2H), 2.92-2.80 (m, 4H) |
| 653 | 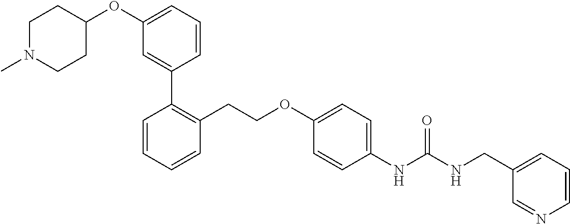 [SMILES: CN1CCC(Oc2cccc(-c3ccccc3CCOc3ccc(NC(=O)NCc4cccnc4)cc3)c2)CC1] | 1-[4-(2-{3′-[(1- Methylpiperidin- 4-yl)oxy] biphenyl-2- yl}ethoxy) phenyl]- 3-(pyridin-3- ylmethyl)urea | LC-MS [M + H]+ 537.2896 | |
| 654 | 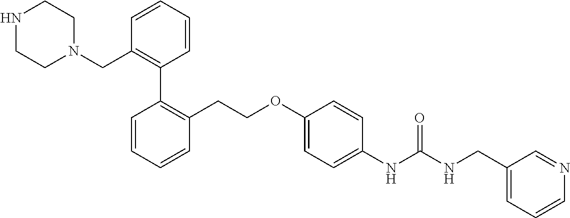 [SMILES: O=C(NCc1cccnc1)Nc1ccc(OCCc2ccccc2-c2ccccc2CN2CCNCC2)cc1] | 1-(4-{2-[2′- (Piperazin-1- ylmethyl) biphenyl-2- yl]ethoxy} phenyl)- 3-(pyridin-3- ylmethyl)urea | LC-MS [M + H]+ 522.2824 | 8.67 (s, 1H), 8.64 (d, 1H), 8.54 (s, 1H), 8.45 (s, 1H), 8.09 (d, 1H), 7.71 (dd, 1H), 7.56 (d, 1H), 7.49-7.34 (m, 4H), 7.30 (t, 1H), 7.25-7.28 (m, 3H), 7.14 (d, 1H), 6.72 (t, 1H), 6.60 (d, 2H), 4.37 (d, 2H), 3.92 (t, 2H), 3.37 (d, 1H), 3.21 (d, 1H), 3.05-2.95 (m, 4H), 2.84- 2.74 (m, 1H), 2.69-2.57 (m, 1H), 2.46-2.35 (m, 4H) |
| 655 | 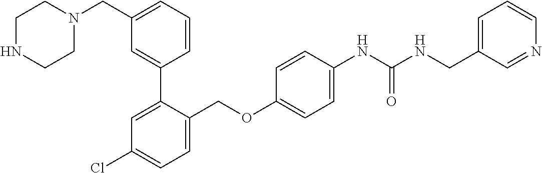 [SMILES: O=C(NCc1cccnc1)Nc1ccc(OCc2ccc(Cl)cc2-c2cccc(CN3CCNCC3)c2)cc1] | 1-(4-{[5-Chloro- 3′-(piperazin-1- ylmethyl) biphenyl- 2- yl]methoxy} phenyl)- 3-(pyridin-3- ylmethyl)urea | LC-MS [M + H]+ 542.2402 | 8.75-8.60 (m, 4H), 8.30 (bs, 1H), 8.16 (d, 1H), 7.98 (d, 1H), 7.76 (t, 1H), 7.68-7.43 (m, 7H), 7.26 (d, 2H), 6.85 (t, 1H), 6.77 (d, 2H), 4.90 (s, 2H), 4.39 (d, 2H), 4.11 (q, 2H), 3.35-2.89 (m, 8H) |
| 656 | 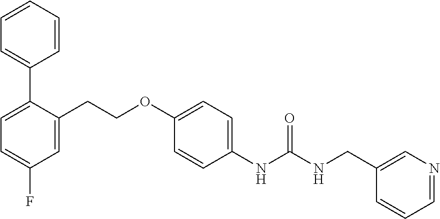 [SMILES: O=C(NCc1cccnc1)Nc1ccc(OCCc2cc(F)ccc2-c2ccccc2)cc1] | 1-{4-[2-(4- Fluorobiphenyl- 2-yl)ethoxy] phenyl}- 3-(pyridin-3- ylmethyl)urea | LC-MS [M + H]+ 442.1923 | 8.51 (s, 1H), 8.45 (d, 1H), 8.40 (s, 1H), 7.69 (dd, 1H), 7.49-7.31 (m, 7H), 7.28-7.19 (m, 3H), 7.14 (td, 1H), 6.66 (d, 2H), 6.59 (t, 1H), 4.29 (d, 2H), 3.99 (t, 2H), 2.95 (t, 2H) |
| 657 | 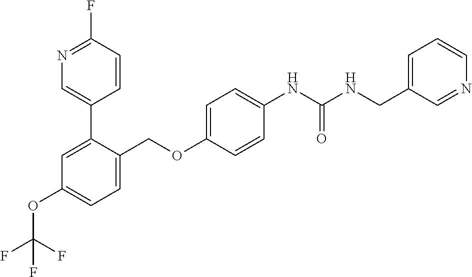 [SMILES: O=C(NCc1cccnc1)Nc1ccc(OCc2ccc(OC(F)(F)F)cc2-c2ccc(F)nc2)cc1] | 1-(4-{[2-(6- Fluoropyridin-3- yl)-4- (trifluoro- methoxy) benzyl]oxy} phenyl)- 3-(pyridin-3- ylmethyl)urea | LC-MS [M + H]+ 513.1529 | |
| 658 | 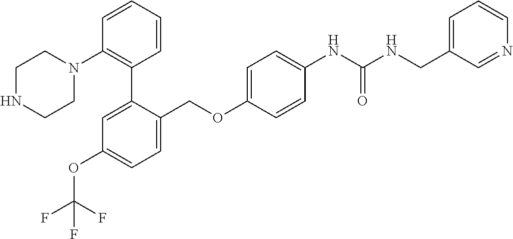 [SMILES: O=C(NCc1cccnc1)Nc1ccc(OCc2ccc(OC(F)(F)F)cc2-c2ccccc2N2CCNCC2)cc1] | 1-(4-{[2′- (Piperazin-1-yl)- 5-(trifluoro- methoxy) biphenyl-2- yl]methoxy} phenyl)- 3-(pyridin-3- ylmethyl)urea | LC-MS [M + H]+ 578.2348 | |
| 659 | 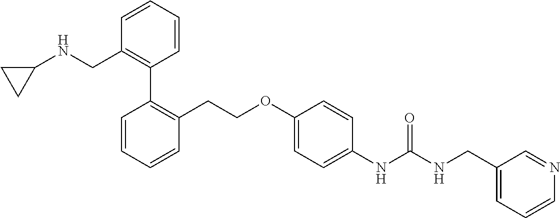 [SMILES: O=C(NCc1cccnc1)Nc1ccc(OCCc2ccccc2-c2ccccc2CNC2CC2)cc1] | 1-[4-(2-{2′- [(Cyclopropyl- amino) methyl]biphenyl- 2- yl}ethoxy) phenyl]- 3-(pyridin-3- ylmethyl)urea | LC-MS [M + H]+ 493.2569 | 9.09 (bs, 1H), 8.83 (bs, 1H), 8.58 (d, 1H), 8.46 (s, 1H), 7.90 (d, 1H), 7.64 (dd, 1H), 7.58-7.30 (m, 7H), 7.25 (d, 1H), 7.22 (d, 2H), 6.64 (q, 1H), 6.63 (d, 2H), 4.34 (d, 2H), 4.16-4.07 (m, 2H), 3.93 (t, 2H), 3.76 (d, 1H), 2.83-2.74 (m, 1H), 2.66- 2.61 (m, 1H), 0.72-0.54 (m, 4H) |
| 660 | 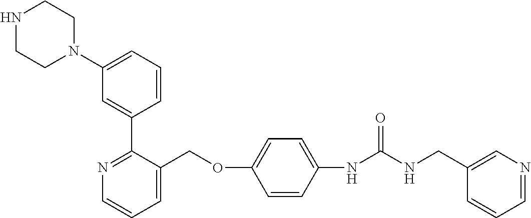 [SMILES: O=C(NCc1cccnc1)Nc1ccc(OCc2cccnc2-c2cccc(N3CCNCC3)c2)cc1] | 1-[4-({2-[3- (Piperazin-1- yl)phenyl]pyridin- 3- yl}methoxy) phenyl]- 3-(pyridin-3- ylmethyl)urea | LC-MS [M + H]+ 495.2487 | |
| 661 | 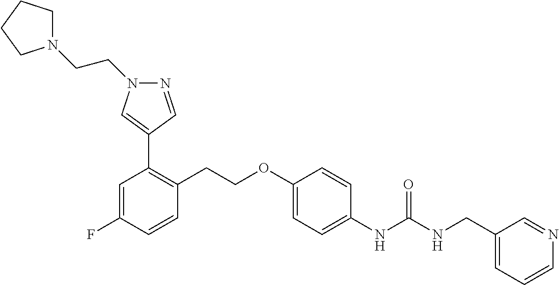 [SMILES: O=C(NCc1cccnc1)Nc1ccc(OCCc2ccc(F)cc2-c2cnn(CCN3CCCC3)c2)cc1] | 1-{4-[2-(4- Fluoro- 2-{1-[2- (pyrrolidin- 1-yl)ethyl]-1H- pyrazol-4- yl}phenyl) ethoxy] phenyl}-3- (pyridin- 3-ylmethyl)urea | LC-MS [M + H]+ 529.2718 | 8.62-8.41 (m, 3H), 8.18 (s, 2H), 8.03 (s, 1H), 7.83 (dd, 1H), 7.68 (s, 1H), 7.49-7.33 (m, 4H), 7.29 (d, 2H), 7.16 (dd, 1H), 7.07 (td, 1H), 6.76 (d, 2H), 6.60 (t, 1H), 4.29 (d, 2H), 4.24 (t, 2H), 4.04 (t, 2H), 3.96 (s, 1H), 2.83 (t, 2H), 2.59-2.52 (m, 2H), 2.46- 2.40 (m, 2H), 1.73-1.57 (m, 4H) |
| 662 | 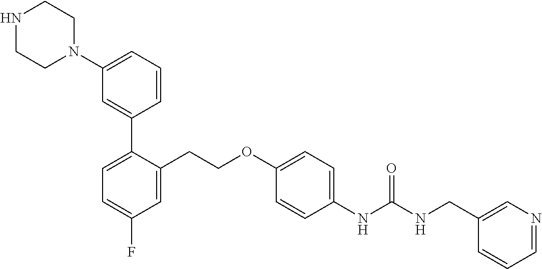 [SMILES: O=C(NCc1cccnc1)Nc1ccc(OCCc2cc(F)ccc2-c2cccc(N3CCNCC3)c2)cc1] | 1-(4-{2-[4- Fluoro- 3′-(piperazin-1- yl)biphenyl-2- yl]ethoxy} phenyl)- 3-(pyridin-3- ylmethyl)urea | LC-MS [M + H]+ 526.2633 | 8.88 (bs, 2H), 8.74-8.62 (m, 3H), 8.16 (d, 1H), 7.77 (t, 1H), 7.37-7.21 (m, 6H), 7.13 (td, 1H), 7.01 (Dd, 1H), 6.95 (s, 1H), 6.88- 6.80 (m, 2H), 6.66 (d, 2H), 4.29 (d, 2H), 4.00 (t, 2H), 3.44-3.35 (m, 4H), 3.23-3.14 (m, 4H), 2.96 (t, 2H) |
| 663 | 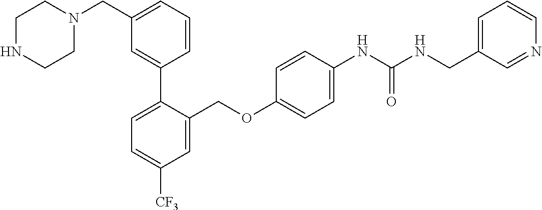 [SMILES: O=C(NCc1cccnc1)Nc1ccc(OCc2cc(C(F)(F)F)ccc2-c2cccc(CN3CCNCC3)c2)cc1] | 1-(4-{[3′- (Piperazin-1- ylmethyl)-4- (trifluoromethyl) biphenyl-2- yl]methoxy} phenyl)- 3-(pyridin-3- ylmethyl)urea | LC-MS [M + H]+ 576.2691 | 8.69-8.53 (m, 5H), 7.98 (s, 1H), 7.92 (bs, 1H), 7.83 (dd, 1H), 7.68-7.38 (m, 6H), 7.26 (d, 2H), 6.79 (d, 2H), 6.72 (t, 1H), 4.97 (s, 2H), 4.35 (d, 2H), 4.10 (q, 2H), 3.22-2.90 (m, 8H) |
| 664 | 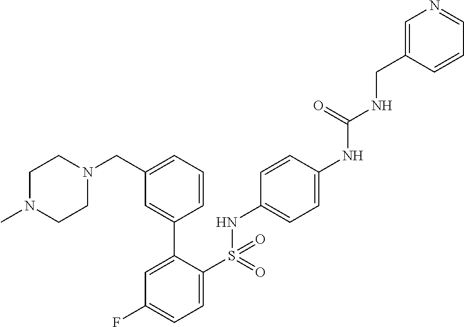 [SMILES: CN1CCN(Cc2cccc(-c3cc(F)ccc3S(=O)(=O)Nc3ccc(NC(=O)NCc4cccnc4)cc3)c2)CC1] | 5-Fluoro-3′-[(4- methylpiperazin- 1- yl)methyl]-N-(4- {[(pyridin-3- ylmethyl) carbamoyl] amino}phenyl) biphenyl-2- sulfonamide | LC-MS [M + H]+ 589.2392 | |
| 665 | 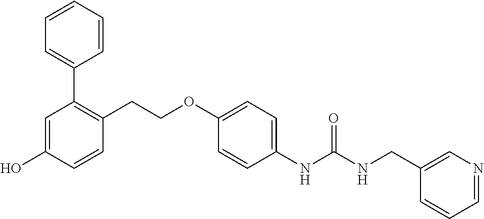 [SMILES: O=C(NCc1cccnc1)Nc1ccc(OCCc2ccc(O)cc2-c2ccccc2)cc1] | 1-{4-[2-(5- Hydroxybiphenyl- 2- yl)ethoxy] phenyl}- 3-(pyridin-3- ylmethyl)urea | LC-MS [M + H]+ 440.2063 | 8.64 (s, 1H), 8.52 (s, 1H), 8.45 (d, 1H), 7.70 (dd, 1H), 7.46-7.26 (m, 11H), 6.97 (d, 2H), 6.88 (dd, 1H), 6.73-6.64 (m, 3H), 4.59 (t, 1H), 4.31 (d, 2H), 3.43 (q, 2H), 2.65 (t, 2H) |
| 666 | 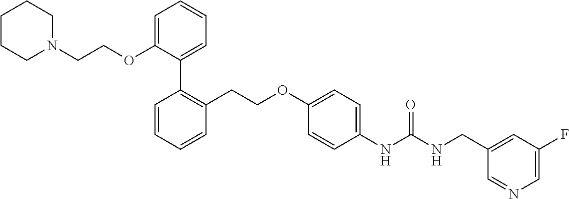 [SMILES: O=C(NCc1cncc(F)c1)Nc1ccc(OCCc2ccccc2-c2ccccc2OCCN2CCCCC2)cc1] | 1-[(5- Fluoropyridin-3- yl)methyl]-3-[4- (2-{2′-[2- (piperidin-1- yl)ethoxy] biphenyl-2- yl}ethoxy) phenyl]urea | LC-MS [M + H]+ 569.2901 | |
| 667 | 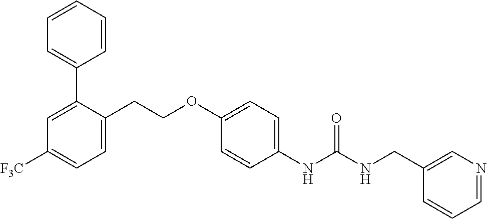 [SMILES: O=C(NCc1cccnc1)Nc1ccc(OCCc2ccc(C(F)(F)F)cc2-c2ccccc2)cc1] | 1-(Pyridin-3- ylmethyl)-3-(4- {2-[5- (trifluoromethyl) biphenyl-2- yl]ethoxy} phenyl)urea | LC-MS [M + H]+ 492.1911 | 8.51 (s, 1H), 8.45 (d, 1H), 8.39 (s, 1H), 7.72 (d, 2H), 7.69 (d, 1H), 7.54-7.33 (m, 8H), 7.22 (d, 2H), 6.65 (d, 2H), 6.57 (d, 1H), 4.29 (d, 2H), 4.01 (t, 2H), 3.04 (t, 2H) |
| 668 | 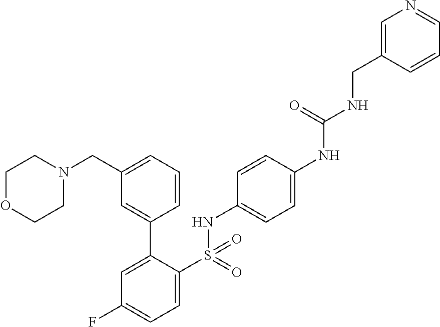 [SMILES: O=C(NCc1cccnc1)Nc1ccc(NS(=O)(=O)c2ccc(F)cc2-c2cccc(CN3CCOCC3)c2)cc1] | 5-Fluoro-3′- (morpholin-4- ylmethyl)-N-(4- {[(pyridin-3- ylmethyl) carbamoyl] amino}phenyl) biphenyl-2- sulfonamide | LC-MS [M + H]+ 576.2087 | |
| 669 | 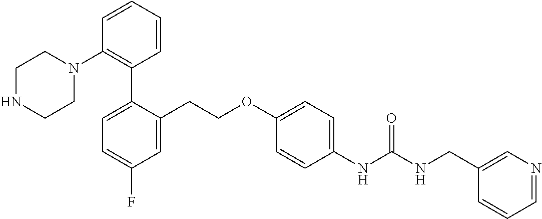 [SMILES: O=C(NCc1cccnc1)Nc1ccc(OCCc2cc(F)ccc2-c2ccccc2N2CCNCC2)cc1] | 1-(4-{2-[4- Fluoro- 2′-(piperazin-1- yl)biphenyl-2- yl]ethoxy} phenyl)- 3-(pyridin-3- ylmethyl)urea | LC-MS [M + H]+ 526.2631 | 8.80 (bs, 1H), 8.63-8.41 (m, 5H), 7.75 (d, 1H), 7.65 (dd, 1H), 7.45-7.10 (m, 13H), 6.67- 6.59 (m, 3H), 4.30 (d, 2H), 4.02-3.88 (m, 2H), 3.01-2.76 (m, 10H) |
| 670 | 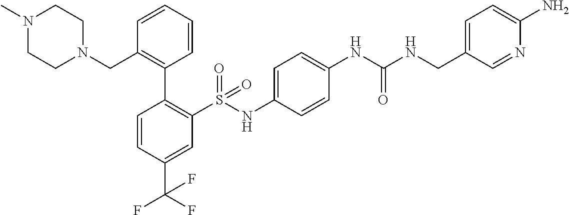 [SMILES: CN1CCN(Cc2ccccc2-c2ccc(C(F)(F)F)cc2S(=O)(=O)Nc2ccc(NC(=O)NCc3ccc(N)nc3)cc2)CC1] | N-[4-({[(6- Aminopyridin-3- yl)methyl] carbamoyl} amino)phenyl]- 2′-[(4- methylpiperazin- 1-yl)methyl]-4- (trifluoromethyl) biphenyl-2- sulfonamide | LC-MS [M + H]+ 654.2473 | |
| 671 | 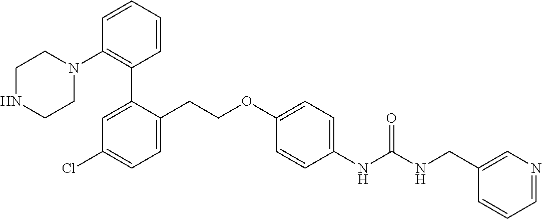 [SMILES: O=C(NCc1cccnc1)Nc1ccc(OCCc2ccc(Cl)cc2-c2ccccc2N2CCNCC2)cc1] | 1-(4-{2-[5- Chloro- 2′-(piperazin-1- yl)biphenyl-2- yl]ethoxy} phenyl)- 3-(pyridin-3- ylmethyl)urea | LC-MS [M + H]+ 542.2310 | 8.51 (s, 1H), 8.46 (s, 1H), 8.45 (dd, 1H), 7.69 (dd, 1H), 7.53-7.15 (m, 11H), 6.64 (t, 1H), 6.59 (d, 2H), 4.29 (d, 2H), 3.96-3.84 (m, 2H), 3.02- 2.73 (m, 2H) |
| 672 | 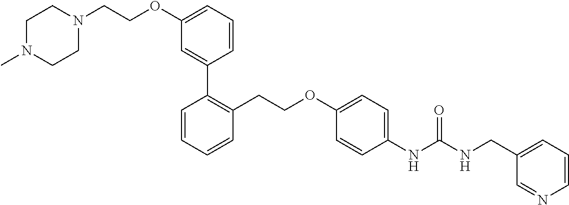 [SMILES: CN1CCN(CCOc2cccc(-c3ccccc3CCOc3ccc(NC(=O)NCc4cccnc4)cc3)c2)CC1] | 1-[4-(2-{3′-[2-(4- Methylpiperazin- 1-yl)ethoxy] biphenyl-2- yl}ethoxy) phenyl]- 3-(pyridin-3- ylmethyl)urea | LC-MS [M + H]+ 566.3146 | |
| 673 | 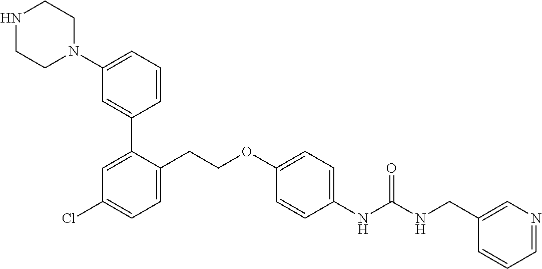 [SMILES: O=C(NCc1cccnc1)Nc1ccc(OCCc2ccc(Cl)cc2-c2cccc(N3CCNCC3)c2)cc1] | 1-(4-{2-[5- Chloro- 3′-(piperazin-1- yl)biphenyl-2- yl]ethoxy} phenyl)- 3-(pyridin-3- ylmethyl)urea | LC-MS [M + H]+ 542.2307 | 8.51 (s, 1H), 8.48-8.43 (m, 2H), 7.69 (dt, 1H), 7.51-7.18 (m, 8H), 7.02 (dd, 1H), 6.97 (s, 1H), 6.84 (d, 1H), 6.66-6.60 (m, 3H), 4.29 (d, 2H), 3.96 (t, 2H), 3.18-3.11 (m, 4H), 2.94 (t, 2H), 2.58- 2.50 (m, 4H) |
| 674 | 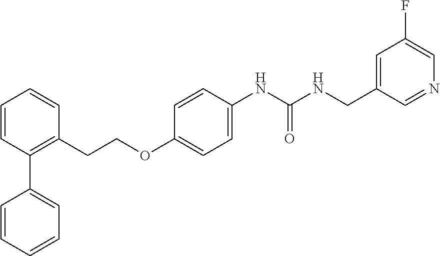 [SMILES: O=C(NCc1cncc(F)c1)Nc1ccc(OCCc2ccccc2-c2ccccc2)cc1] | 1-{4-[2- (Biphenyl- 2- yl)ethoxy] phenyl}- 3-[(5- fluoropyridin- 3-yl)methyl]urea | LC-MS [M + H]+ 442.1912 | |
| 675 | 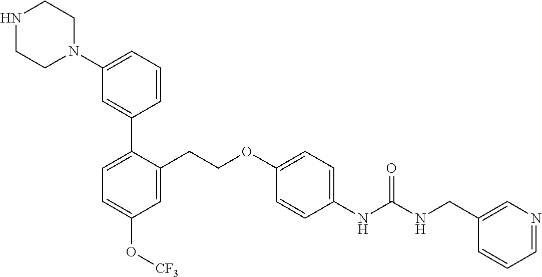 [SMILES: O=C(NCc1cccnc1)Nc1ccc(OCCc2cc(OC(F)(F)F)ccc2-c2cccc(N3CCNCC3)c2)cc1] | 1-(4-{2-[3′- (Piperazin-1-yl)- 4-(trifluoro- methoxy) biphenyl-2- yl]ethoxy} phenyl)- 3-(pyridin-3- ylmethyl)urea | LC-MS [M + H]+ 592.2563 | 8.89 (bs, 2H), 8.75-8.64 (m, 3H), 8.17 (d, 1H), 7.77 (t, 1H), 7.49 (s, 1H), 7.38-7.26 (m, 3H), 7.03 (d, 2H), 7.03 (dd, 1H),6.98 (s, 1H), 6.85 (d, 2H), 6.65 (d, 2H), 4.40 (d, 2H), 4.00 (t, 2H), 3.40-3.36 (m, 4H), 3.23- 3.15 (m, 4H), 2.99 (t, 2H) |
| 676 | 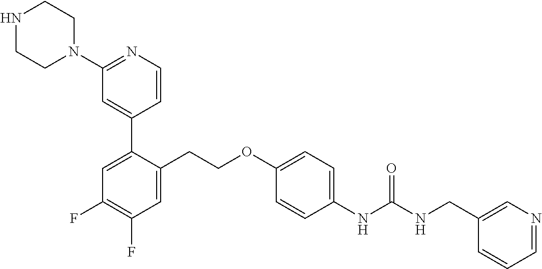 [SMILES: O=C(NCc1cccnc1)Nc1ccc(OCCc2cc(F)c(F)cc2-c2ccnc(N3CCNCC3)c2)cc1] | 1-[4-(2-{4,5- Difluoro-2-[2- (piperazin-1- yl)pyridin-4- yl]phenyl}ethoxy) phenyl]-3- (pyridin- 3-ylmethyl)urea | LC-MS [M + H]+ 545.2469 | 8.51 (s, 1H), 8.45 (d, 1H), 8.40 (s, 1H), 8.16 (d, 1H), 7.70 (dd, 1H), 7.60 (s, 1H), 7.40-7.28 (m, 3H), 7.24 (d, 2H), 6.77 (s, 1H), 6.69 (d, 2H), 6.67-6.54 (m, 2H), 4.29 (d, 2H), 4.03 (t, 2H), 3.06-2.76 (m, 10H) |
| 677 | 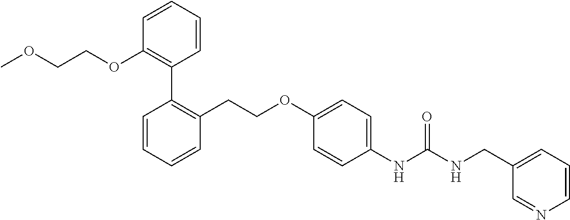 [SMILES: COCCOc1ccccc1-c1ccccc1CCOc1ccc(NC(=O)NCc2cccnc2)cc1] | 1-(4-{2-[2′-(2- Methoxyethoxy) biphenyl-2- yl]ethoxy} phenyl)- 3-(pyridin-3- ylmethyl)urea | LC-MS [M + Na]+ 520.2206 | |
| 678 | 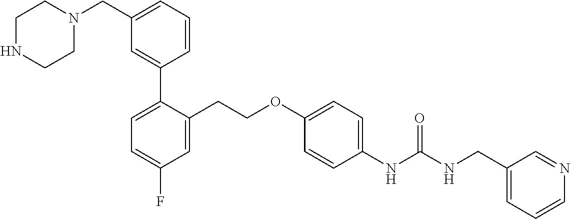 [SMILES: O=C(NCc1cccnc1)Nc1ccc(OCCc2cc(F)ccc2-c2cccc(CN3CCNCC3)c2)cc1] | 1-(4-{2-[4- Fluoro- 3′-(piperazin-1- ylmethyl) biphenyl-2- yl]ethoxy} phenyl)- 3-(pyridin-3- ylmethyl)urea | LC-MS [M + H]+ 540.2899 | 9.34 (bs, 1H), 9.24 (bs, 1H), 8.78-8.68 (m, 3H), 8.65 (d, 1H), 8.33 (bs, 2H), 8.03 (d, 1H), 7.85 (dd, 1H), 7.64-7.27 (m, 7H), 7.24 (d, 2H), 7.17 (td, 1H), 6.87 (t, 1H), 6.70 (d, 2H), 4.41 (d, 2H), 4.12 (q, 2H), 4.03 (t, 2H), 3.42-3.15 (m, 8H), 2.98 (t, 2H) |
| 679 | 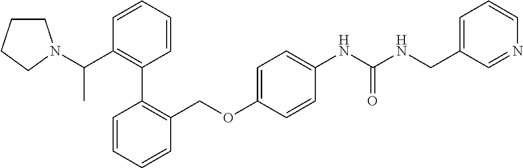 [SMILES: CC(c1ccccc1-c1ccccc1COc1ccc(NC(=O)NCc2cccnc2)cc1)N1CCCC1] | 1-(Pyridin-3- ylmethyl)-3-[4- ({2′- [1-(pyrrolidin-1- yl)ethyl]biphenyl- 2- yl}methoxy) phenyl]urea | LC-MS [M + H]+ 507.2830 | |
| 680 | 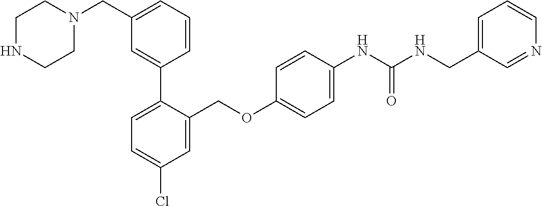 [SMILES: O=C(NCc1cccnc1)Nc1ccc(OCc2cc(Cl)ccc2-c2cccc(CN3CCNCC3)c2)cc1] | 1-(4-{[4-Chloro- 3′-(piperazin-1- ylmethyl) biphenyl-2- yl]methoxy} phenyl)- 3-(pyridin-3- ylmethyl)urea | LC-MS [M + H]+ 542.2411 | 8.73-8.61 (m, 3H), 8.28 (bs, 1H), 8.13 (d, 1H), 7.97 (d, 1H), 7.74 (d, 1H), 7.70-7.37 (m, 7H), 7.26 (d, 2H), 6.83 (t, 1H), 6.79 (d, 2H), 4.92 (s, 2H), 4.39 (d, 2H), 4.11 (q, 2H), 3.36-3.20 (m, 8H) |
| 681 | 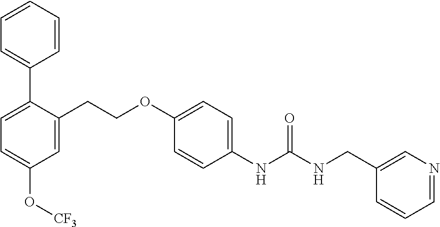 [SMILES: O=C(NCc1cccnc1)Nc1ccc(OCCc2cc(OC(F)(F)F)ccc2-c2ccccc2)cc1] | 1-(Pyridin-3- ylmethyl)-3-(4- {2-[4- (trifluoro- methoxy) biphenyl-2- yl]ethoxy} phenyl) urea | LC-MS [M + H]+ 508.1869 | 8.51 (s, 1H), 8.45 (d, 1H), 8.39 (s, 1H), 7.69 (d, 1H), 7.54-7.29 (m, 7H), 7.22 (d, 2H), 6.65 (d, 2H), 6.58 (t, 1H), 6.56 (s, 1H), 4.29 (d, 2H), 3.99 (t, 2H), 2.99 (t, 2H) |
| 682 | 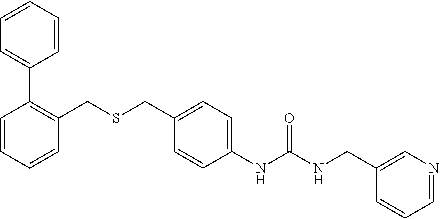 [SMILES: O=C(NCc1cccnc1)Nc1ccc(CSCc2ccccc2-c2ccccc2)cc1] | 1-(4-{[(Biphenyl- 2-ylmethyl) sulfanyl] methyl}phenyl)- 3-(pyridin-3- ylmethyl)urea | LC-MS [M + H]+ 440.1790 | |
| 683 | 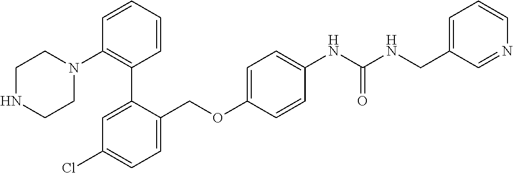 [SMILES: O=C(NCc1cccnc1)Nc1ccc(OCc2ccc(Cl)cc2-c2ccccc2N2CCNCC2)cc1] | 1-(4-{[5-Chloro- 2′-(piperazin-1- yl)biphenyl-2- yl]methoxy} phenyl)- 3-(pyridin-3- ylmethyl)urea | LC-MS [M + H]+ 528.2161 | |
| 684 | 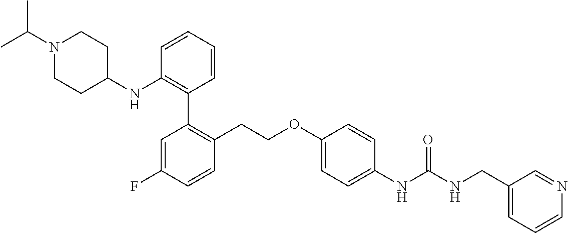 [SMILES: CC(C)N1CCC(Nc2ccccc2-c2cc(F)ccc2CCOc2ccc(NC(=O)NCc3cccnc3)cc2)CC1] | 1-{4-[2-(5- Fluoro- 2′-{[1-(propan-2- yl)piperidin-4- yl]amino} biphenyl-2- yl)ethoxy] phenyl}- 3-(pyridin-3- ylmethyl)urea | LC-MS [M + H]+ 582.3308 | 8.53 (s, 1H), 8.49 (d, 1H), 8.41 (s, 1H), 8.05 (bs, 1H), 7.76 (dd, 1H), 7.55 (d, 1H), 7.33-7.22 (m, 2H), 7.19 (d, 2H), 6.98 (t, 1H, 6.86 (d, 1H), 6.76 (t, 1H), 6.68- 6.57 (m, 4H), 4.30 (d, 2H), 3.87 (t, 1H), 3.83-3.73 (m, 2H), 3.12- 2.86 (m, 6H), 1.49-1.13 (m, 10H) |
| 685 | 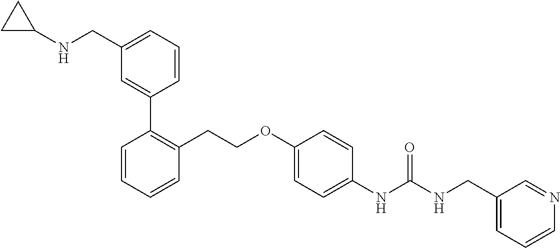 [SMILES: O=C(NCc1cccnc1)Nc1ccc(OCCc2ccccc2-c2cccc(CNC3CC3)c2)cc1] | 1-[4-(2-{3′- [(Cyclopropyl- amino) methyl]biphenyl- 2- yl}ethoxy) phenyl]- 3-(pyridin-3- ylmethyl)urea | LC-MS [M + H]+ 493.2582 | 8.98 (s, 1H), 8.62 (d, 1H), 8.53 (s, 1H), 7.99 (d, 1H), 7.62 (dd, 1H), 7.56-7.30 (m, 7H), 7.26-7.18 (m, 3H), 6.72 (t, 1H), 6.68 (d, 2H), 4.35 (d, 2H), 4.30 (t, 2H), 3.97 (t, 2H), 2.98 (t, 2H), 2.74-2.64 (m, 1H), 0.83- 0.69 (m, 4H) |
| 686 | 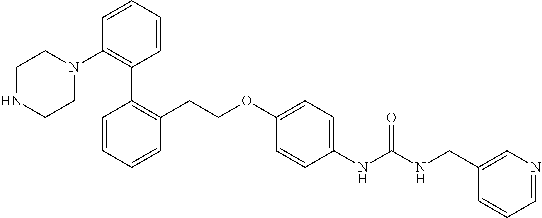 [SMILES: O=C(NCc1cccnc1)Nc1ccc(OCCc2ccccc2-c2ccccc2N2CCNCC2)cc1] | 1-(4-{2-[2′- (Piperazin-1- yl)biphenyl-2- yl]ethoxy} phenyl)- 3-(pyridin-3- ylmethyl)urea | LC-MS [M + H]+ 508.2720 | 8.60 (s, 1H), 8.56 (d, 1H), 8.48 (s, 1H), 7.92 (d, 1H), 7.56 (dd, 1H), 7.49-7.13 (m, 10H), 6.66 (t, 1H), 6.58 (d, 2H), 4.33 (d, 2H), 3.98-3.82 (m, 2H), 3.00- 2.70 (m, 10H) |
| 687 | 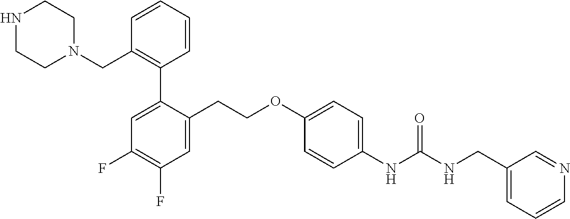 [SMILES: O=C(NCc1cccnc1)Nc1ccc(OCCc2cc(F)c(F)cc2-c2ccccc2CN2CCNCC2)cc1] | 1-(4-{2-[4,5- Difluoro-2′- (piperazin-1- ylmethyl) biphenyl-2- yl]ethoxy} phenyl)- 3-(pyridin-3- ylmethyl)urea | LC-MS [M + H]+ 558.2764 | 9.31 (bs, 1H), 8.77-8.60 (m, 3H), 8.36-8.10 (m, 2H), 8.00 (d, 1H), 7.80 (bs, 1H), 7.65- 7.25 (m, 3H), 7.22 (d, 2H), 6.83 (t, 1H), 6.63 (d, 2H), 4.40 (d, 1.5H), 4.34 (d, 0.5H), 4.11 (q, 2H), 3.95 (q, 2H), 3.19-2.69 (m, 10H) |
| 688 | 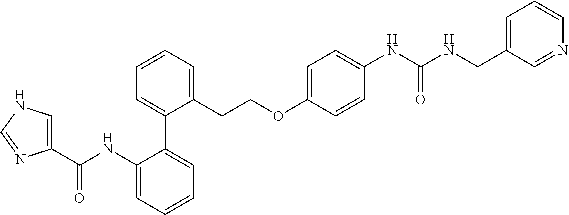 [SMILES: O=C(NCc1cccnc1)Nc1ccc(OCCc2ccccc2-c2ccccc2NC(=O)c2c[nH]cn2)cc1] | N-{2′-[2-(4- {[(Pyridin-3- ylmethyl) carbamoyl] amino}phenoxy) ethyl]biphenyl- 2-yl}-1H- imidazole- 4-carboxamide | LC-MS [M + H]+ 533.2339 | |
| 689 | 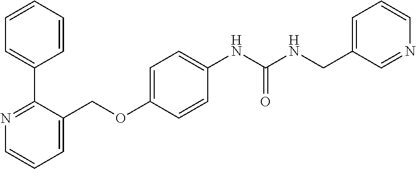 [SMILES: O=C(NCc1cccnc1)Nc1ccc(OCc2cccnc2-c2ccccc2)cc1] | 1-{4-[(2- Phenylpyridin-3- yl)methoxy] phenyl}- 3-(pyridin-3- ylmethyl)urea | LC-MS [M + H]+ 411.1817 | |
| 690 | 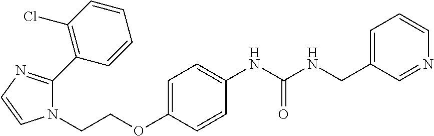 [SMILES: O=C(NCc1cccnc1)Nc1ccc(OCCn2ccnc2-c2ccccc2Cl)cc1] | 1-(4-{2-[2-(2- Chlorophenyl)- 1H-imidazol-1- yl]ethoxy} phenyl)- 3-(pyridin-3- ylmethyl)urea | LC-MS [M + H]+ 448.1549 | |
| 691 | 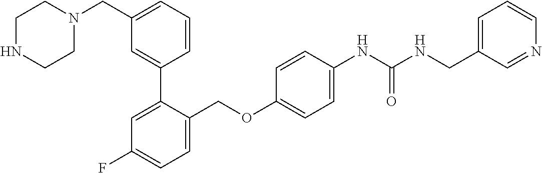 [SMILES: O=C(NCc1cccnc1)Nc1ccc(OCc2ccc(F)cc2-c2cccc(CN3CCNCC3)c2)cc1] | 1-(4-{[5-Fluoro- 3′-(piperazin-1- ylmethyl) biphenyl-2- yl]methoxy} phenyl)- 3-(pyridin-3- ylmethyl)urea | LC-MS [M + H]+ 526.2705 | 8.71-8.58 (m, 3H), 8.24 (bs, 1H), 8.06 (d, 1H), 7.94 (d, 1H), 7.73-7.44 (m, 6H), 7.34- 7.21 (m, 3H), 6.83-6.74 (m, 3H), 4.86 (s, 2H), 4.37 (d, 2H), 4.10 (q, 2H), 3.34-3.16 (m, 8H) |
| 692 | 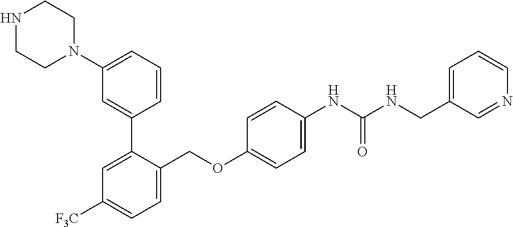 [SMILES: O=C(NCc1cccnc1)Nc1ccc(OCc2ccc(C(F)(F)F)cc2-c2cccc(N3CCNCC3)c2)cc1] | 1-(4-{[3′- (Piperazin-1-yl)- 5-(trifluoro- methyl) biphenyl-2- yl]methoxy} phenyl)- 3-(pyridin-3- ylmethyl)urea | LC-MS [M + H]+ 562.2417 | 8.51 (s, 1H), 8.48-8.43 (m, 2H), 7.85-7.76 (m, 2H), 7.69 (dt, 1H), 7.62 (s, 1H), 7.38- 7.30 (m, 2H), 7.27 (d, 2H), 7.02-6.97 (m, 2H), 6.87 (d, 1H), 6.78 (d, 2H), 6.61 (t, 1H), 4.96 (s, 2H), 4.29 (d, 2H), 3.14-3.07 (m, 4H), 2.93- 2.85 (m, 4H) |
| 693 | 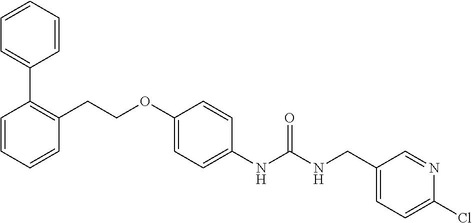 [SMILES: O=C(NCc1ccc(Cl)nc1)Nc1ccc(OCCc2ccccc2-c2ccccc2)cc1] | 1-{4-[2- (Biphenyl- 2- yl)ethoxy] phenyl}- 3-[(6- chloropyridin-3- yl)methyl]urea | LC-MS [M + H]+ 458.1610 | 8.53 (s, 1H), 8.33 (d, 1H), 7.77 (dd, 1H), 7.50-7.25 (m, 9H), 7.24-7.18 (m, 3H), 6.69 (t, 1H), 6.63 (d, 2H), 4.28 (d, 2H), 3.95 (t, 2H), 2.96 (t, 2H) |
| 694 | 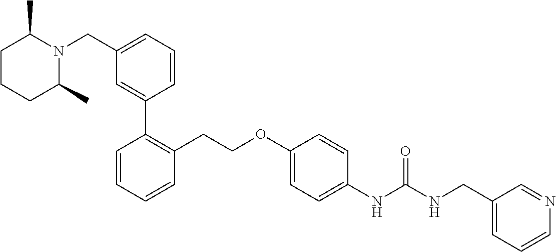 [SMILES: C[C@@H]1CCC[C@H](C)N1Cc1cccc(-c2ccccc2CCOc2ccc(NC(=O)NCc3cccnc3)cc2)c1] | 1-{4-[2-(3′- {[(2R,6S)-2,6- Dimethyl- piperidin-1- yl]methyl} biphenyl-2- yl)ethoxy] phenyl}- 3-(pyridin-3- ylmethyl)urea | LC-MS [M + H]+ 549.3228 | 9.10 (bs, 1H), 9.00 (bs, 1H), 8.62 (s, 1H), 8.58 (d, 1H), 8.51 (s, 1H), 7.96 (d, 1H), 7.65-7.31 (m, 7H), 7.27-7.18 (m, 3H), 6.72- 6.60 (m, 3H), 4.48 (d, 2H), 4.35 (d, 2H), 3.95 (q, 1H), 3.47 (bs, 1H), 3.08 (bs, 1H), 2.98 (t, 2H), 1.83-1.49 (m, 6H), 1.46 (d, 3H), 1.26 (d, 3H) |
| 695 | 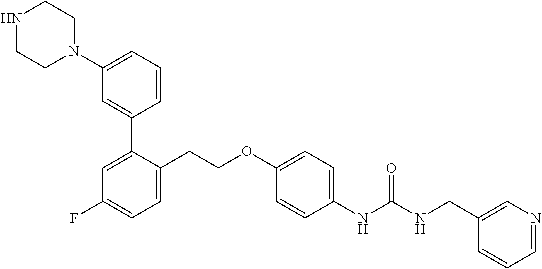 [SMILES: O=C(NCc1cccnc1)Nc1ccc(OCCc2ccc(F)cc2-c2cccc(N3CCNCC3)c2)cc1] | 1-(4-{2-[5- Fluoro- 3′-(piperazin-1- yl)biphenyl-2- yl]ethoxy} phenyl)- 3-(pyridin-3- ylmethyl)urea | LC-MS [M + H]+ 526.2645 | 8.82 (bs, 2H), 8.70-8.60 (m, 3H), 8.09 (d, 1H), 7.70 (t, 1H), 7.50 (dd, 1H), 7.35 (t, 1H), 7.26-7.14 (m, 3H), 7.06- 6.97 (m, 3H), 6.86 (d, 1H), 6.78 (t, 1H), 6.64 (d, 2H), 4.38 (d, 2H), 3.95 (t, 2H), 3.45-3.36 (m, 4H), 3.25-3.15 (m, 4H), 2.94 (t, 2H) |
| 696 | 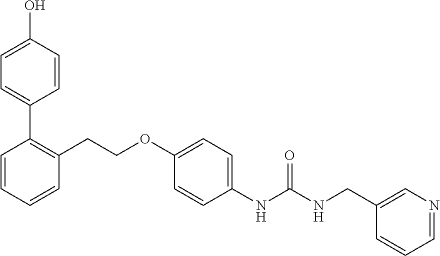 [SMILES: O=C(NCc1cccnc1)Nc1ccc(OCCc2ccccc2-c2ccc(O)cc2)cc1] | 1-{4-[2-(4′- Hydroxy- biphenyl-2- yl)ethoxy] phenyl}- 3-(pyridin-3- ylmethyl)urea | LC-MS [M + H]+ 440.1926 | 9.52 (s, 1H), 8.51 (s, 1H), 8.45 (d, 1H), 8.38 (s, 1H), 7.69 (dd, 1H), 7.46-7.19 (m, 6H), 7.15 (d, 2H), 6.83 (d, 2H), 6.66 (d, 2H), 6.58 (t, 2H), 4.29 (d, 2H), 3.94 (t, 2H), 2.96 (t, 2H) |
| 697 | 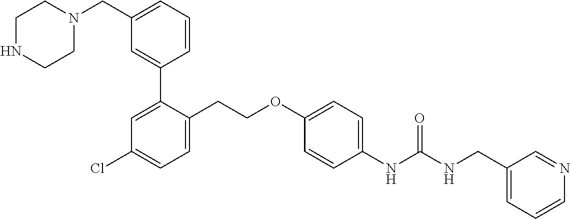 [SMILES: O=C(NCc1cccnc1)Nc1ccc(OCCc2ccc(Cl)cc2-c2cccc(CN3CCNCC3)c2)cc1] | 1-(4-{2-[5- Chloro- 3′-(piperazin-1- ylmethyl) biphenyl-2- yl]ethoxy} phenyl)- 3-(pyridin-3- ylmethyl)urea | LC-MS [M + H]+ 556.2606 | 9.37 (bs, 1H), 9.28 (bs, 1H), 8.77 (s, 1H), 8.74 (s, 1H), 8.72 (d, 1H), 8.66 (d, 1H), 8.36 (bs, 1H), 8.27 (d, 1H), 8.05 (d, 1H), 7.86 (dd, 1H), 7.67-7.30 (m, 8H), 7.24 (d, 2H), 6.89 (t, 1H), 6.68 (d, 2H), 4.42 (d, 2H), 4.12 (q, 2H), 3.98 (t, 2H), 3.40-3.18 (m, 8H), 2.96 (t, 2H) |
| 698 | 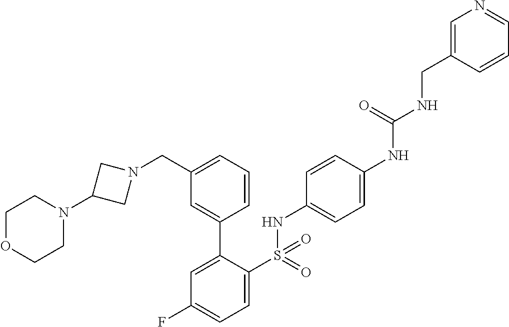 [SMILES: O=C(NCc1cccnc1)Nc1ccc(NS(=O)(=O)c2ccc(F)cc2-c2cccc(CN3CC(N4CCOCC4)C3)c2)cc1] | 5-Fluoro-3′-{[3- (morpholin-4- yl)azetidin-1- yl]methyl}-N-(4- {[(pyridin-3- ylmethyl) carbamoyl] amino}phenyl) biphenyl-2- sulfonamide | LC-MS [M + H]+ 631.2516 | |
| 699 | 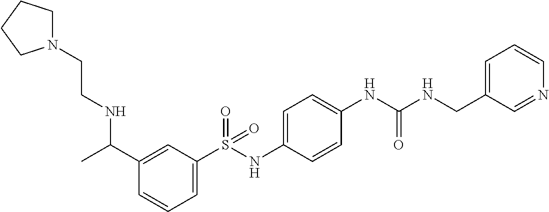 [SMILES: CC(NCCN1CCCC1)c1cccc(S(=O)(=O)Nc2ccc(NC(=O)NCc3cccnc3)cc2)c1] | N-(4-{[(Pyridin- 3-ylmethyl) carbamoyl] amino}phenyl)- 3-(1-{[2- (pyrrolidin-1- yl)ethyl]amino} ethyl)benzene- sulfonamide | LC-MS [M + H]+ 523.2494 | |
| 700 | 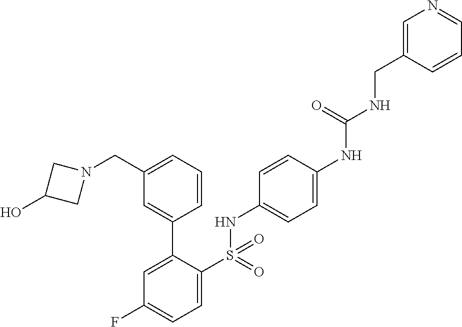 [SMILES: O=C(NCc1cccnc1)Nc1ccc(NS(=O)(=O)c2ccc(F)cc2-c2cccc(CN3CC(O)C3)c2)cc1] | 5-Fluoro-3′-[(3- hydroxyazetidin- 1- yl)methyl]-N-(4- {[(pyridin-3- ylmethyl) carbamoyl] amino}phenyl) biphenyl-2- sulfonamide | LC-MS [M + H]+ 562.1922 | |
| 701 | 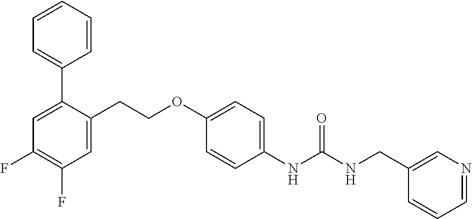 [SMILES: O=C(NCc1cccnc1)Nc1ccc(OCCc2cc(F)c(F)cc2-c2ccccc2)cc1] | 1-{4-[2-(4,5- Difluorobiphenyl- 2- yl)ethoxy] phenyl}- 3-(pyridin-3- ylmethyl)urea | LC-MS [M + H]+ 460.1827 | 8.51 (s, 1H), 8.45 (d, 1H), 8.41 (s, 1H), 7.69 (d, 1H), 7.58 (dd, 1H), 7.51-7.26 (m, 8H), 7.22 (d, 2H), 6.65 (d, 2H), 6.60 (t, 2H), 4.29 (d, 2H), 3.98 (t, 2H), 2.91 (t, 2H) |
| 702 | 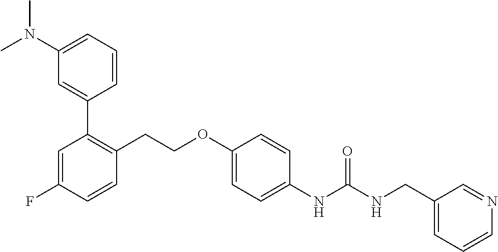 [SMILES: CN(C)c1cccc(-c2cc(F)ccc2CCOc2ccc(NC(=O)NCc3cccnc3)cc2)c1] | 1-(4-{2-[3′- (Dimethyl- amino)- 5-fluorobiphenyl- 2- yl]ethoxy} phenyl)- 3-(pyridin-3- ylmethyl)urea | LC-MS [M + H]+ 485.2349 | 8.51 (s, 1H), 8.45 (d, 1H), 8.38 (s, 1H), 7.61 (dt, 1H), 7.47 (dd, 1H), 7.35 (dd, 1H), 7.29-7.11 (m, 4H), 7.02 (dd, 1H), 6.76 (dd, 1H), 6.67-6.54 (m, 4H), 4.29 (d, 2H), 3.96 (t, 2H), 2.95 (t, 2H), 2.91 (s, 6H) |
| 703 | 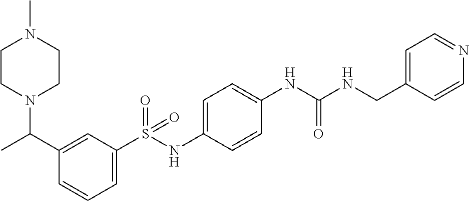 [SMILES: CC(c1cccc(S(=O)(=O)Nc2ccc(NC(=O)NCc3ccncc3)cc2)c1)N1CCN(C)CC1] | 3-[1-(4- Methylpiperazin- 1-yl)ethyl]-N-(4- {[(pyridin-3- ylmethyl) carbamoyl] amino}phenyl) benzene- sulfonamide | LC-MS [M + H]+ 509.2341 | |
| 704 | 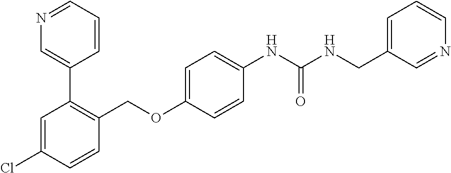 [SMILES: O=C(NCc1cccnc1)Nc1ccc(OCc2ccc(Cl)cc2-c2cccnc2)cc1] | 1-(4-{[4-Chloro- 2-(pyridin-3- yl)benzyl]oxy} phenyl)-3- (pyridin- 3-ylmethyl)urea | LC-MS [M + H]+ 445.143 | |
| 705 | 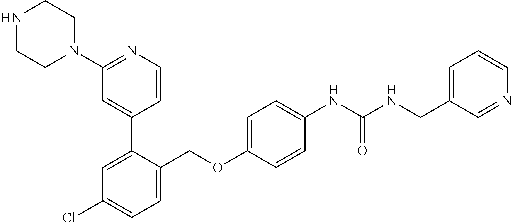 [SMILES: O=C(NCc1cccnc1)Nc1ccc(OCc2ccc(Cl)cc2-c2ccnc(N3CCNCC3)c2)cc1] | 1-[4-({4-Chloro- 2-[2-(piperazin-1- yl)pyridin-4- yl]benzyl}oxy) phenyl]-3- (pyridin- 3-ylmethyl)urea | LC-MS [M + H]+ 529.2094 | |
| 706 | 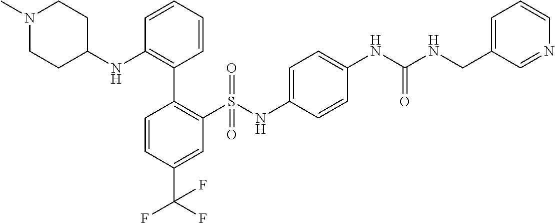 [SMILES: CN1CCC(Nc2ccccc2-c2ccc(C(F)(F)F)cc2S(=O)(=O)Nc2ccc(NC(=O)NCc3cccnc3)cc2)CC1] | 2′-[(1- Methylpiperidin- 4- yl)amino]-N-(4- {[(pyridin-3- ylmethyl) carbamoyl] amino}phenyl)- 4- (trifluoromethyl) biphenyl-2- sulfonamide | LC-MS [M + H]+ 639.2330 | |
| 707 | 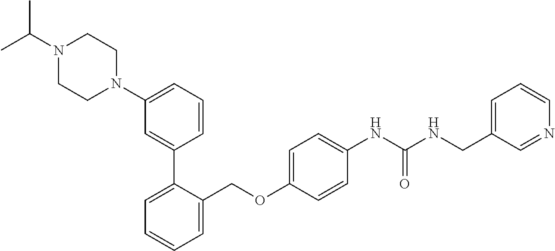 [SMILES: CC(C)N1CCN(c2cccc(-c3ccccc3COc3ccc(NC(=O)NCc4cccnc4)cc3)c2)CC1] | 1-[4-({3′-[4- (Propan-2- yl)piperazin-1- yl]biphenyl-2- yl}methoxy) phenyl]- 3-(pyridin-3- ylmethyl)urea | LC-MS [M + H]+ 536.303 | |
| 708 | 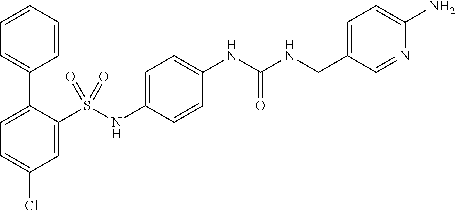 [SMILES: Nc1ccc(CNC(=O)Nc2ccc(NS(=O)(=O)c3cc(Cl)ccc3-c3ccccc3)cc2)cn1] | N-[4-({[(6- Aminopyridin-3- yl)methyl] carbamoyl} amino)phenyl]- 4-chlorobiphenyl- 2-sulfonamide | LC-MS [M + H]+ 508.1189 | |
| 709 | 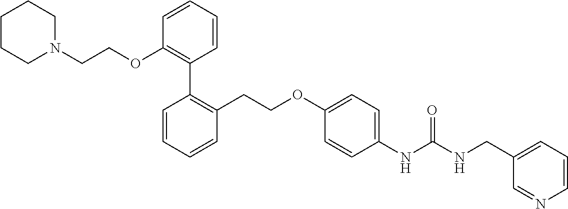 [SMILES: O=C(NCc1cccnc1)Nc1ccc(OCCc2ccccc2-c2ccccc2OCCN2CCCCC2)cc1] | 1-[4-(2-{2′-[2- (Piperidin-1- yl)ethoxy] biphenyl-2- yl}ethoxy) phenyl]- 3-(pyridin-3- ylmethyl)urea | LC-MS [M + H]+ 551.301 | |
| 710 | 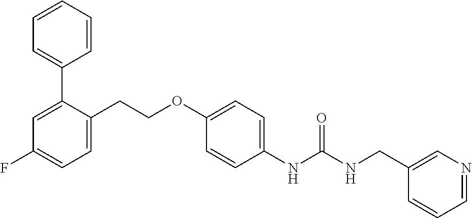 [SMILES: O=C(NCc1cccnc1)Nc1ccc(OCCc2ccc(F)cc2-c2ccccc2)cc1] | 1-{4-[2-(5- Fluorobiphenyl- 2-yl)ethoxy] phenyl}- 3-(pyridin-3- ylmethyl)urea | LC-MS [M + H]+ 442.1924 | 8.51 (s, 1H), 8.45 (d, 1H), 8.40 (s, 1H), 7.69 (d, 1H), 7.56-7.33 (m, 8H), 7.25-7.33 (m, 3H), 7.04 (dd, 1H), 6.63 (d, 2H), 6.59 (t, 1H), 4.29 (d, 2H), 3.94 (t, 2H), 2.93 (t, 2H) |
| 711 | 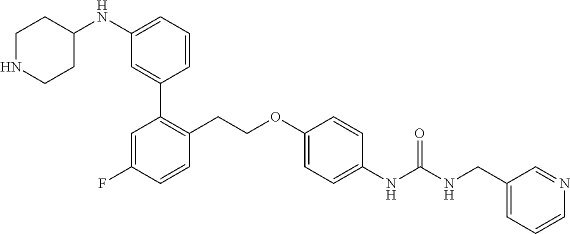 [SMILES: O=C(NCc1cccnc1)Nc1ccc(OCCc2ccc(F)cc2-c2cccc(NC3CCNCC3)c2)cc1] | 1-(4-{2-[5- Fluoro- 3′-(piperidin-4- ylamino) biphenyl-2- yl]ethoxy} phenyl)- 3-(pyridin-3- ylmethyl)urea | LC-MS [M + H]+ 540.2823 | 8.53-8.47 (m, 2H), 8.45 (d, 1H), 7.69 (dt, 1H), 7.46 (dd, 1H), 7.35 (dd, 1H), 7.23 (d, 2H), 7.19-7.08 (m, 2H), 6.98 (dd, 2H), 6.71 (t, 1H), 6.65 (d, 2H), 6.60 (d, 1H), 6.50 (s, 1H), 6.44 (d, 1H), 5.59 (d, 1H), 4.29 (d, 2H), 3.94 (t, 2H), 3.30-3.19 (m, 1H), 2.94 (t, 2H), 2.91-2.82 (m, 2H), 2.49- 2.41 (m, 2H), 1.89-1.79 (m, 2H), 1.26-1.19 (m, 2H) |
| 712 | 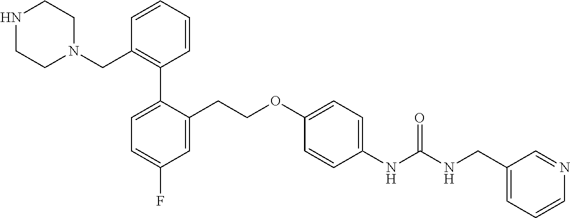 [SMILES: O=C(NCc1cccnc1)Nc1ccc(OCCc2cc(F)ccc2-c2ccccc2CN2CCNCC2)cc1] | 1-(4-{2-[4- Fluoro- 2′-(piperazin-1- ylmethyl) biphenyl-2- yl]ethoxy} phenyl)- 3-(pyridin-3- ylmethyl)urea | LC-MS [M + H]+ 540.2856 | 9.45 (bs, 1H), 8.83-8.61 (m, 4H), 8.40 (bs, 1H), 8.29 (dd, 1H), 8.09 (d, 1H), 7.88 (q, 1H), 7.62 (dd, 1H), 7.46 (t, 1H), 7.38 (d, 1H), 7.23 (d, 2H), 7.16 (td, 1H), 7.06 (t, 1H), 6.92 (t, 1H), 6.64 (d, 2H), 4.43 (d, 1H), 4.37 (d, 1H), 4.13 (q, 2H), 3.96 (q, 2H), 3.28-2.71 (m, 10H) |
| 713 | 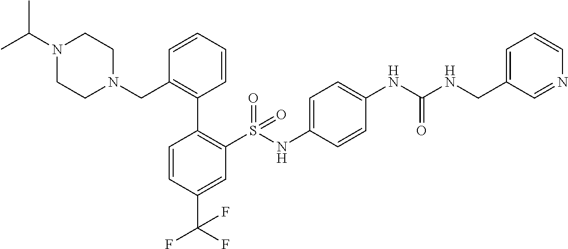 [SMILES: CC(C)N1CCN(Cc2ccccc2-c2ccc(C(F)(F)F)cc2S(=O)(=O)Nc2ccc(NC(=O)NCc3cccnc3)cc2)CC1] | 2′-{[4-(Propan-2- yl)piperazin-1- yl]methyl}-N-(4- {[(pyridin-3- ylmethyl) carbamoyl] amino}phenyl)- 4- (trifluoromethyl) biphenyl-2- sulfonamide | LC-MS [M + H]+ 667.2649 | |
| 714 | 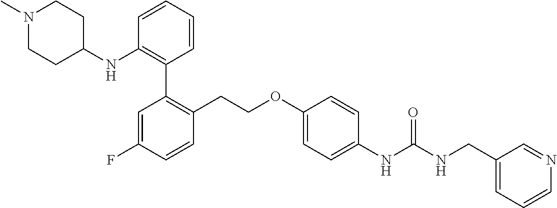 [SMILES: CN1CCC(Nc2ccccc2-c2cc(F)ccc2CCOc2ccc(NC(=O)NCc3cccnc3)cc2)CC1] | 1-[4-(2-{5- Fluoro- 2′-[(1- methylpiperidin- 4-yl)amino] biphenyl-2- yl}ethoxy) phenyl]- 3-(pyridin-3- ylmethyl)urea | LC-MS [M + H]+ 554.2964 | 8.56 (s, 1), 8.51 )d, 1H), 8.42 (s, 1H), 7.81 (d, 1H), 7.52 (dd, 1H), 7.47 (bs, 1H), 7.31-7.17 (m, 4H), 7.02- 6.92 (m, 2H), 6.84 (d, 1H), 6.74 (t, 1H), 6.62 (t, 1H), 6.54 (d, 2H), 4.31 (d, 2H), 3.86 (t, 2H), 3.08-2.91 (m, 2H), 2.88-2.76 (m, 1H), 2.73 (d, 3H), 2.10- 1.91 (m, 4H), 1.50-1.34 (m, 4H) |
| 715 | 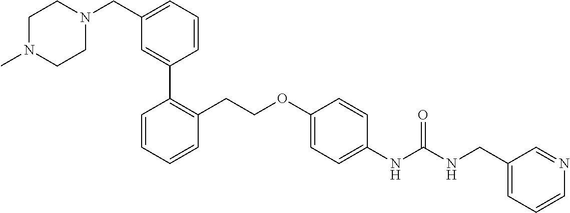 [SMILES: CN1CCN(Cc2cccc(-c3ccccc3CCOc3ccc(NC(=O)NCc4cccnc4)cc3)c2)CC1] | 1-[4-(2-{3′-[(4- Methylpiperazin- 1-yl)methyl] biphenyl-2- yl}ethoxy) phenyl]- 3-(pyridin-3- ylmethyl)urea | LC-MS [M + H]+ 536.3051 | 8.66 (m, 2H), 8.52 (d, 1H), 8.02 (bs, 1H), 7.65 (bs, 1H), 7.50-7.28 (m, 7H), 7.25-7.18 (m, 3H), 6.70 (t, 1H), 6.65 (d, 2H), 4.36 (d, 2H), 3.95 (t, 2H), 3.78-3.68 (m, 2H), 3.30- 3.00 (bs, 4H), 2.97 (t, 2H), 2.72 (s, 3H) |
| 716 | 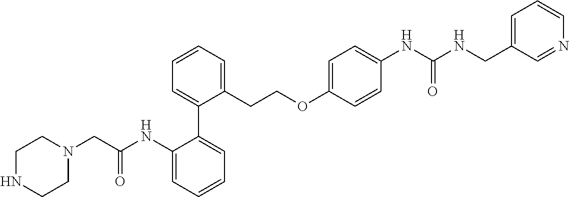 [SMILES: O=C(CN1CCNCC1)Nc1ccccc1-c1ccccc1CCOc1ccc(NC(=O)NCc2cccnc2)cc1] | 2-(Piperazin-1- yl)-N-{2′-[2-(4- {[(pyridin-3- ylmethyl) carbamoyl] amino}phenoxy) ethyl]biphenyl-2- yl}acetamide | LC-MS [M + H]+ 565.2922 | |
| 717 | 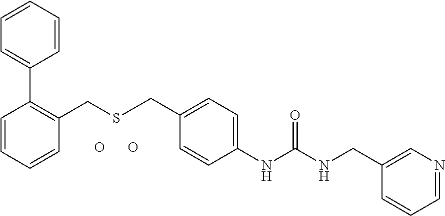 | 1-(4-{[(Biphenyl- 2- ylmethyl) sulfonyl] methyl}phenyl)- 3-(pyridin-3- ylmethyl)urea | LC-MS [M + H]+ 472.7 167 | |
| 718 | 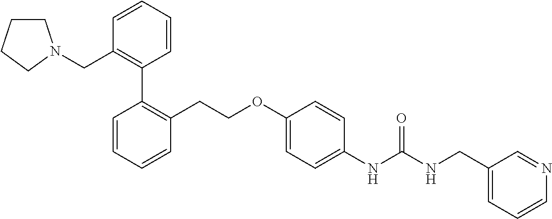 [SMILES: O=C(NCc1cccnc1)Nc1ccc(OCCc2ccccc2-c2ccccc2CN2CCCC2)cc1] | 1-(Pyridin-3- ylmethyl)-3-(4- {2- [2′-(pyrrolidin-1- ylmethyl) biphenyl-2- yl]ethoxy} phenyl) urea | LC-MS [M + H]+ 507.2735 | 8.57 (bs, 1H), 8.45 (s, 1H), 7.88 (d, 1H), 7.73 (d, 1H), 7.61-7.31 (m, 5H), 7.26-7.18 (m, 3H), 6.62 (d, 2H), 4.33 (d, 2H), 4.00-3.85 (m, 4H), 3.5- 3.40 (m, 2H), 3.31-3.22 (m, 2H), 2.97-2.85 (m, 1H), 2.85- 2.73 (m, 1H), 2.64-2.56 (m, 1H), 1.85-1.72 (m, 4H) |
| 719 | 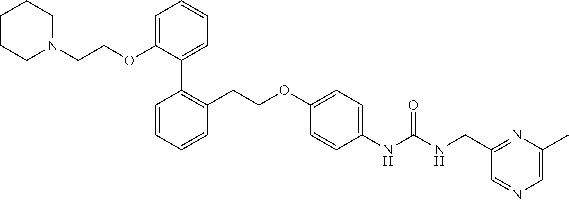 [SMILES: Cc1cncc(CNC(=O)Nc2ccc(OCCc3ccccc3-c3ccccc3OCCN3CCCCC3)cc2)n1] | 1-[(6- Methylpyrazin- 2-yl)methyl]-3- [4-(2- {2′-[2-(piperidin- 1-yl)ethoxy] biphenyl-2- yl}ethoxy) phenyl]urea | LC-MS [M + H]+ 566.310 | |
| 720 | 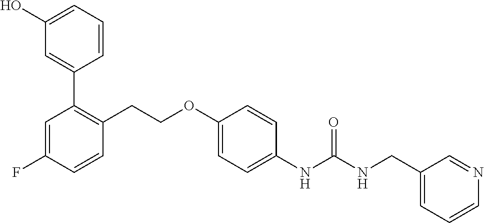 [SMILES: O=C(NCc1cccnc1)Nc1ccc(OCCc2ccc(F)cc2-c2cccc(O)c2)cc1] | 1-{4-[2-(5- Fluoro-3′- hydroxy- biphenyl-2- yl)ethoxy] phenyl}- 3-(pyridin-3- ylmethyl)urea | LC-MS [M + H]+ 458.1826 | 9.61 (s, 1H), 8.55-8.43 (m, 3H), 8.39 (s, 1H), 7.74-7.63 (m, 2H), 7.48 (dd, 1H), 7.40- 7.11 (m, 5H), 7.01 (dd ,1H), 6.85-6.71 (m, 3H), 6.65 (d, 2H), 6.58 (t, 1H), 4.29 (d, 2H), 3.93 (t, 2H), 2.93 (t, 2H) |
| 721 | 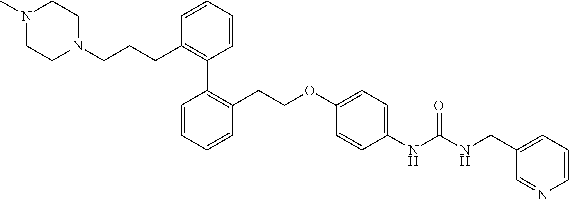 [SMILES: CN1CCN(CCCc2ccccc2-c2ccccc2CCOc2ccc(NC(=O)NCc3cccnc3)cc2)CC1] | 1-[4-(2-{2′-[2-(4- Methylpiperazin- 1-yl)ethoxy] biphenyl-2- yl}ethoxy) phenyl]- 3-(pyridin-3- ylmethyl)urea | LC-MS [M + H]+ 566.3132 | |
| 722 | 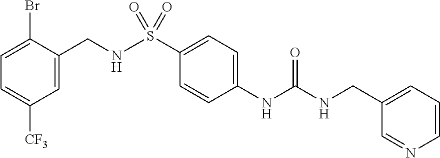 [SMILES: O=C(NCc1cccnc1)Nc1ccc(S(=O)(=O)NCc2cc(C(F)(F)F)ccc2Br)cc1] | N-[2-Bromo-5- (trifluoromethyl) benzyl]-4- {[(pyridin- 3- ylmethyl) carbamoyl] amino}benzene sulfonamide | LC-MS [M + H]+ 543.0308 | |
| 723 | 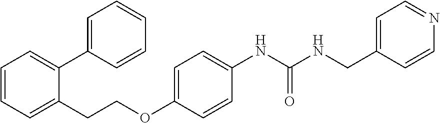 [SMILES: O=C(NCc1ccncc1)Nc1ccc(OCCc2ccccc2-c2ccccc2)cc1] | 1-{4-[2- (Biphenyl-2- yl)ethoxy] benzyl}- 3-pyridin-4-ylurea | LC-MS [M + H]+ 424.2033 | |
| 724 | 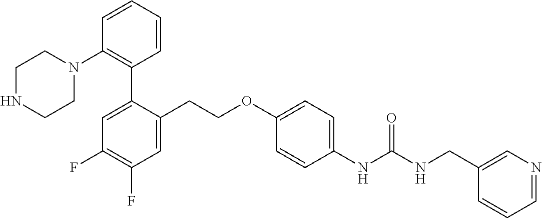 [SMILES: O=C(NCc1cccnc1)Nc1ccc(OCCc2cc(F)c(F)cc2-c2ccccc2N2CCNCC2)cc1] | 1-(4-{2-[4,5- Difluoro-2′- (piperazin-1- yl)biphenyl-2- yl]ethoxy} phenyl)- 3-(pyridin-3- ylmethyl)urea | LC-MS [M + H]+ 544.2513 | 8.68-8.57 (m, 4H), 8.06 (d, 1H), 7.68 (t, 1H), 7.56 (td, 1H), 7.45-7.31 (m, 2H), 7.25- 7.14 (m, 5H), 6.75 (t, 1H), 6.62 (d, 2H), 4.37 (d, 2H), 4.00-3.86 (m, 2H), 3.02-2.71 (m, 10H) |
| 725 | 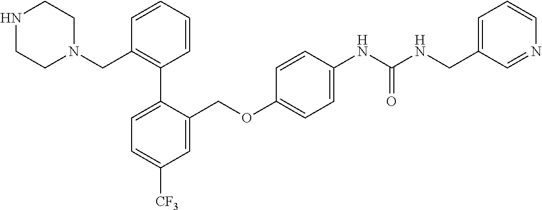 [SMILES: O=C(NCc1cccnc1)Nc1ccc(OCc2cc(C(F)(F)F)ccc2-c2ccccc2CN2CCNCC2)cc1] | 1-(4-{[2′- (Piperazin-1- ylmethyl)-4- (trifluoromethyl) biphenyl-2- yl]methoxy} phenyl)- 3-(pyridin-3- ylmethyl)urea | LC-MS [M + H]+ 576.2671 | n/a |
| 726 | 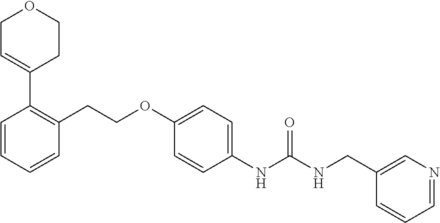 [SMILES: O=C(NCc1cccnc1)Nc1ccc(OCCc2ccccc2C2=CCOCC2)cc1] | 1-(4-{2-[2-(3,6- Dihydro-2H- pyran-4- yl)phenyl]ethoxy} phenyl)-3- (pyridin- 3-ylmethyl)urea | LC-MS [M + H]+ 430.2150 | 8.72 (s, 1H), 8.69 (d, 1H), 8.57 (s, 1H), 8.21 (d, 1H), 7.80 (dd, 1H), 7.39-7.09 (m, 6H), 6.81 (d, 2H), 6.71 (t, 1H), 5.66 (p, 1H), 4.40 (d, 2H), 4.18-4.21 (m, 2H), 4.08 (t, 2H), 3.80 (t, 2H), 3.01 (t, 2H), 2.31-2.25 (m, 2H) |
| 727 | 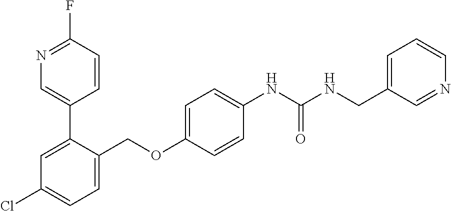 [SMILES: O=C(NCc1cccnc1)Nc1ccc(OCc2ccc(Cl)cc2-c2ccc(F)nc2)cc1] | 1-(4-{[4-Chloro- 2-(6- fluoropyridin- 3- yl)benzyl]oxy} phenyl)-3- (pyridin- 3-ylmethyl)urea | LC-MS [M + H]+ 463.1318 | |
| 728 | 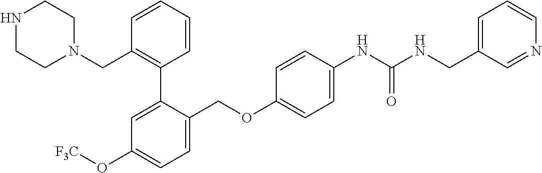 [SMILES: O=C(NCc1cccnc1)Nc1ccc(OCc2ccc(OC(F)(F)F)cc2-c2ccccc2CN2CCNCC2)cc1] | 1-(4-{[2′- (Piperazin-1- ylmethyl)-5- (trifluoro- methoxy) biphenyl-2- yl]methoxy} phenyl)- 3-(pyridin-3- ylmethyl)urea | LC-MS [M + H]+ 592.2604 | 8.69-8.44 (m, 5H), 8.20 (bs, 1H), 7.91 (d, 1H), 7.52-7.28 (m, 5H), 7.27- 7.19 (m, 3H), 6.69 (d, 2H), 4.65 (s, 2H), 4.34 (d, 2H), 4.10 (q, 2H), 3.32-2.85 (m, 8H) |
| 729 | 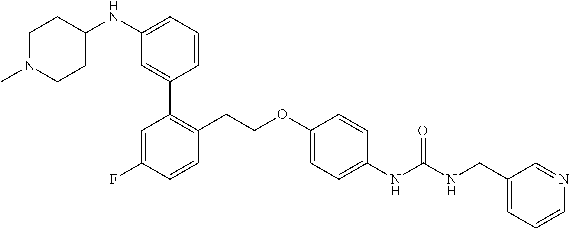 [SMILES: CN1CCC(Nc2cccc(-c3cc(F)ccc3CCOc3ccc(NC(=O)NCc4cccnc4)cc3)c2)CC1] | 1-[4-(2-{5- Fluoro- 3′-[(1- methylpiperidin- 4-yl)amino] biphenyl-2- yl}ethoxy) phenyl]- 3-(pyridin-3- ylmethyl)urea | 554.1516 (M + H) | 9.18 (bs, 1H), 8.62 (s, 1H), 8.59 (d, 1H), 8.49 (s, 1H), 7.97 (d, 1H), 7.61 (d, 1H), 7.48 (dd, 1H), 7.22 (d, 2H), 7.20-7.12 (m, 2H), 7.00 (dd, 1H), 6.73-6.61 (m, 4H), 6.60- 6.49 (m, 2H), 4.35 (d, 2H), 3.95 (t, 2H), 3.44-3.33 (m, 2H), 3.31-3.12 (m, 1H), 3.05- 2.89 (m, 4H), 2.72 (d, 3H), 2.16-2.07 (m, 4H) |
| 730 | 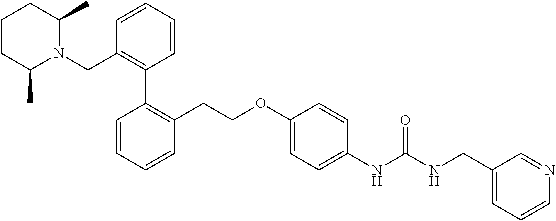 [SMILES: C[C@@H]1CCC[C@H](C)N1Cc1ccccc1-c1ccccc1CCOc1ccc(NC(=O)NCc2cccnc2)cc1] | 1-{4-[2-(2′- {[(2R,6S)-2,6- Dimethyl- piperidin-1- yl]methyl} biphenyl-2- yl)ethoxy] phenyl}- 3-(pyridin-3- ylmethyl)urea | LC-MS [M + H]+ 549.3204 | 9.04 (bs, 1H), 8.61 (d, 1H), 8.51 (s, 1H), 8.35 (s, 1H), 7.97 (d, 1H), 7.72-7.33 (m, 7H), 7,23 (d, 2H), 6.69 (d, 2H), 4.35 (d, 2H), 4.25-3.86 (m, 4H), 3.46 (bs, 1H), 3.23 (bs, 1H), 2.91- 2.77 (m, 1H), 2.72-2.62 (m, 1H), 1.82-1.34 (m, 4H), 1.21- 0.95 (m, 8H) |
| 731 | 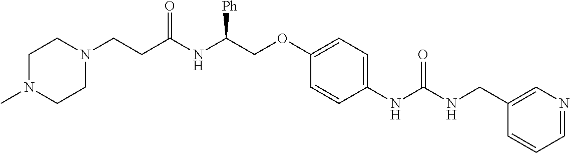 [SMILES: CN1CCN(CCC(=O)N[C@H](COc2ccc(NC(=O)NCc3cccnc3)cc2)c2ccccc2)CC1] | 3-(4- Methylpiperazin- 1-yl)-N-[(1S)-1- phenyl-2-(4- {[(pyridin-3- ylmethyl) carbamoyl] amino}phenoxy) ethyl] propanamide | LC-MS [M + H]+ 517.2932 | 8.75 (d, 1H), 8.51 (s, 1H), 8.46 (s, 1H), 8.44 (dd, 1H), 7.70 (d, 1H), 7.43-7.24 (m, 7H), 6.82 (d, 2H), 6.64 (t, 1H), 5.18 (q, 1H), 4.29 (d, 2H), 4.07 (t, 2H), 2.97 (q, 1H), 2.43-2.20 (m, 8H), 2.11 (s, 3H) |
| 732 | 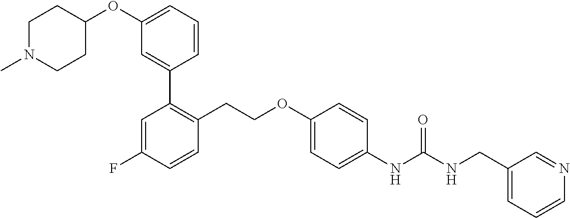 [SMILES: CN1CCC(Oc2cccc(-c3cc(F)ccc3CCOc3ccc(NC(=O)NCc4cccnc4)cc3)c2)CC1] | 1-[4-(2-{5- Fluoro-3-[(1- methylpiperidin- 4-yl)oxy] biphenyl-2- yl}ethoxy) phenyl]- 3-(pyridin-3- ylmethyl)urea | LC-MS [M + H]+ 555.2821 | 8.51 (s, 1H), 8.45 (dd, 1H), 8.41(s, 1H), 7.70 (dt, 1H), 7.50 (dd, 1H), 7.39-7.32 (m, 3H), 7.25-7.15 (m, 3H), 7.06 (dd, 1H), 7.01- 6.93 (m, 2H), 6.91 (d, 1H), 6.64 (d, 2H), 6.59 (t, 1H), 4.45-4.37 (m, 1H), 4.30 (d, 2H), 3.95 (t, 2H), 2.93 (t, 2H), 2.79-2.53 (m, 4H), 2.15 (s, 3H), 1.78- 1.55 (m, 4H) |
| 733 | 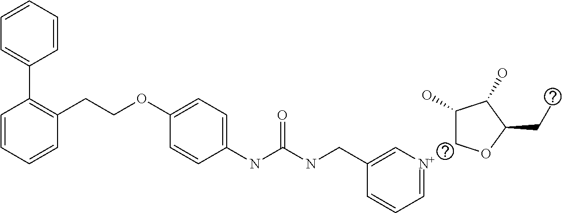 | 3-{[({4-[2- (Biphenyl-2- yl)ethoxy]phenyl} carbamoyl)amino] methyl}-1- ribofuranosylpyri- dinium | 556.2552 [M + H]+ | 9.16 (s, 1H), 9.10 (d, 1H), 8.74 (s, 1H). 8.56 (d, 1H), 8.19 (dd, 1H), 7.50-7.17 (m, 12H), 6.87 (t, 1H), 6.64 (d, 2H), 6.11 (d, 1H), 5.96 (d, 1H), 5.53 (d, 1H), 5.41 (t, 1H), 4.50 (d, 2H), 4.25-4.19 (m, 2H), 3.95 (t, 2H), 3.85-3.66 (m, 2H), 2.96 (t, 2H) |
| 734 | 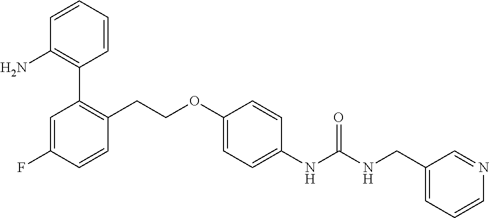 [SMILES: Nc1ccccc1-c1cc(F)ccc1CCOc1ccc(NC(=O)NCc2cccnc2)cc1] | 1-{4-[2-(2′- Amino- 5-fluorobiphenyl- 2- yl)ethoxy] phenyl}- 3-(pyridin-3- ylmethyl)urea | LC-MS [M + H]+ 457.2054 | 8.51 (s, 1H), 8.45 (d, 1H), 8.37 (s, 1H), 7.69 (d, 1H), 7.49 (dd, 1H), 7.35 (dd, 1H), 7.24-7.08 (m, 4H), 6.93 (td, 2H), 6.77 (d, 1H), 6.65 (t, 1H), 6.61- 6.553 (m, 3H), 4.60 (s, 2H), 4.29 (d, 2H), 3.89 (q, 2H), 2.82 (q, 2H) |
| 735 | 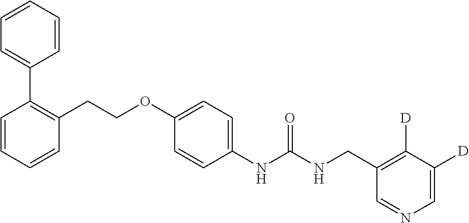 [SMILES: [2H]c1cncc(CNC(=O)Nc2ccc(OCCc3ccccc3-c3ccccc3)cc2)c1[2H]] | 1-{4-[2- (Biphenyl-2- yl)ethoxy] phenyl}- 3-[(4,5- ~2~H _2_) pyridin- 3-ylmethyl]urea | LC-MS [M + H]+ 426.2127 | |
| 736 | 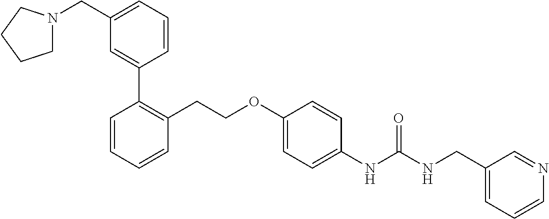 [SMILES: O=C(NCc1cccnc1)Nc1ccc(OCCc2ccccc2-c2cccc(CN3CCCC3)c2)cc1] | 1-(Pyridin-3- ylmethyl)-3-(4- {2- [3′-(pyrrolidin-1- ylmethyl) biphenyl-2- yl]ethoxy} phenyl)urea | LC-MS [M + H]+ 507.2737 | 9.78 (s, 1H), 8.68-8.57 (m, 2H), 8.54 (s, 1H), 8.02 (d, 1H), 7.65 (dd, 1 H), 7.60-7.30 (m, 7H), 7.26- 7.20 (m, 3H), 6.74 (t, 1H), 6.66 (d, 2H), 4.39 (d, 2H), 4.36 (d, 2H), 3.96 (t, 2H), 3.40-3.28 (m, 2H), 3.15-3.03 (m, 2H), 2.98 (t, 2H), 2.04-1.92 (m, 2H), 1.87- 1.75 (m, 2H) |
| 737 | 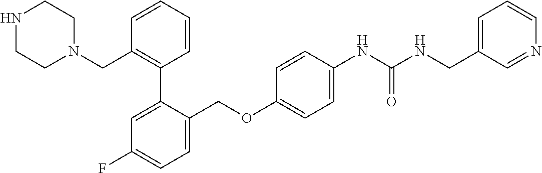 [SMILES: O=C(NCc1cccnc1)Nc1ccc(OCc2ccc(F)cc2-c2ccccc2CN2CCNCC2)cc1] | 1-(4-{[5-Fluoro- 2′- (piperazin-1- ylmethyl) biphenyl- 2- yl]methoxy} phenyl)- 3-(pyridin-3- ylmethyl)urea | LC-MS [M + H]+ 526.2692 | 8.73-.55 (m, 5H), 8.25 (bs, 1H), 8.08 (bs, 1H), 7.65 (d, 1H), 7.69 (bs, 1H), 7.63 (dd, 1H), 7.53 (dd, 1H), 7.45-7.25 (m, 3H), 7.22 (d, 2H), 7.17- 7.10 (m, 1H), 6.76 (t, 1H), 6.68 (d, 2H), 4.59 (q, 2H), 4.37 (d, 2H), 4.11 (q, 2H), 3.37-2.82 (m, 8H) |
| 738 | 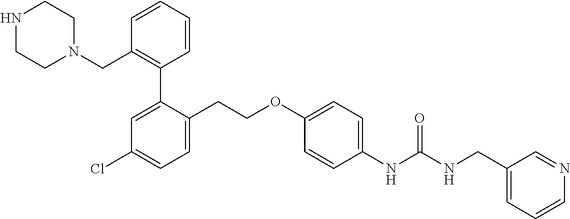 [SMILES: O=C(NCc1cccnc1)Nc1ccc(OCCc2ccc(Cl)cc2-c2ccccc2CN2CCNCC2)cc1] | 1-(4-{2-[5- Chloro- 2′-(piperazin-1- ylmethyl) biphenyl-2- yl]ethoxy} phenyl)- 3-(pyridin-3- ylmethyl)urea | LC-MS [M + H]+ 556.2568 | 9.11 (bs, 1H), 8.71-8.49 (m, 4H), 8.22 (bs, 1H), 7.92 (d, 1H), 7.70-7.35 (m, 4H), 7.29- 7.17 (m, 3H), 6.68 (t, 1H), 6.61 (d, 2H), 4.34 (d, 1H), 4.30 (d, 1H), 4.10 (q, 2H), 3.91 (q, 2H), 3.23-2.73 (m, 10H) |
| 739 | 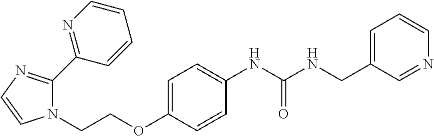 [SMILES: O=C(NCc1cccnc1)Nc1ccc(OCCn2ccnc2-c2ccccn2)cc1] | 1-(4-{2-[2- (Pyridin-2-yl)- 1H-imidazol-1- yl]ethoxy} phenyl)- 3-(pyridin-3- ylmethyl)urea | LC-MS [M + H]+ 415.1878 | |
| 740 | 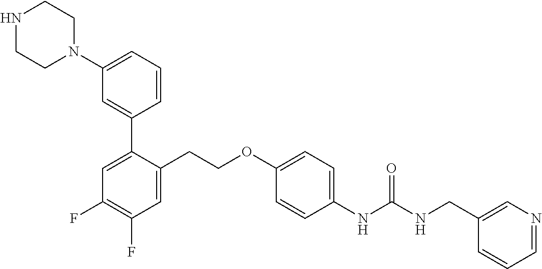 [SMILES: O=C(NCc1cccnc1)Nc1ccc(OCCc2cc(F)c(F)cc2-c2cccc(N3CCNCC3)c2)cc1] | 1-(4-{2-[4,5- Difluoro-3′- (piperazin-1- yl)biphenyl-2- yl]ethoxy} phenyl)- 3-(pyridin-3- ylmethyl)urea | LC-MS [M + H]+ 544.2540 | 8.71 (bs, 2H), 8.59 (s, 1H), 8.54 (d, 1H), 8.50 (s, 1H), 7.88 (d, 1H), 7.58 (dd, 1H), 7.52 (bs, 1H), 7.41-7.25 (m, 2H), 7.23 (d, 2H), 7.03 (dd, 1H), 6.97 (s, 1H), 6.85 (d, 1H), 6.71-6.62 (m, 3H), 4.33 (d, 2H), 3.99 (t, 2H), 3.43-3.33 (m, 4H), 3.26- 3.16 (m, 4H), 2.93 (t, 2H) |
| 741 | 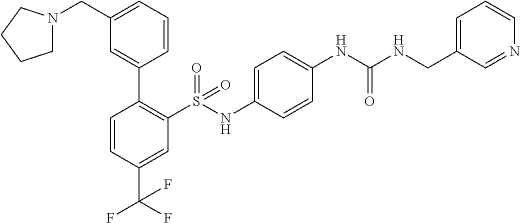 [SMILES: O=C(NCc1cccnc1)Nc1ccc(NS(=O)(=O)c2cc(C(F)(F)F)ccc2-c2cccc(CN3CCCC3)c2)cc1] | N-(4-{[(Pyridin- 3-ylmethyl) carbamoyl] amino}phenyl)- 3′-(pyrrolidin-1- ylmethyl)-4- (trifluoromethyl) biphenyl-2- sulfonamide | LC-MS [M + H]+ 610.2086 | |
| 742 | 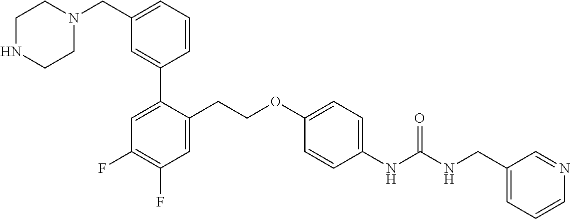 [SMILES: O=C(NCc1cccnc1)Nc1ccc(OCCc2cc(F)c(F)cc2-c2cccc(CN3CCNCC3)c2)cc1] | 1-(4-{2-[4,5- Difluoro-3′- (piperazin-1- ylmethyl) biphenyl-2- yl]ethoxy} phenyl)- 3-(pyridin-3- ylmethyl)urea | LC-MS [M + H]+ 558.2820 | 9.40 (bs, 1H), 9.33 (bs, 1H), 8.80 (s, 1H), 8.75 (s, 1H), 8.75 (d, 1H), 8.73 (d, 1H), 8.66 (d, 1H), 8.38 (bs, 1H), 8.28 (d, 1H), 8.06 (d, 2H), 7.87 (dd, 1), 7.68-7.34 (m, 7H), 7.24 (d, 2H), 6.91 (t, 1H), 6.70 (d, 2H), 4.42 (d, 2H), 4.12 (q, 2H), 4.02 (t, 2H), 3.43-3.20 (m, 8H), 2.95 (t, 2H) |
| 743 | 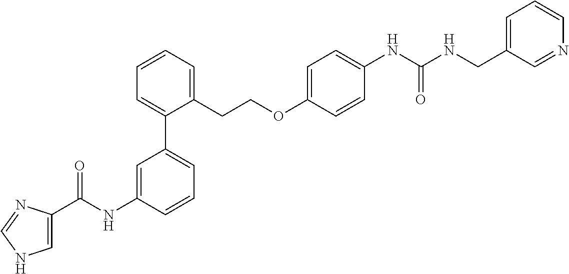 [SMILES: O=C(NCc1cccnc1)Nc1ccc(OCCc2ccccc2-c2cccc(NC(=O)c3c[nH]cn3)c2)cc1] | N-{2′-[2-(4- {[(Pyridin-3- ylmethyl) carbamoyl] amino}phenoxy) ethyl]biphenyl- 3-yl}-1H- imidazole- 4-carboxamide | LC-MS [M + H]+ | |
| 744 | 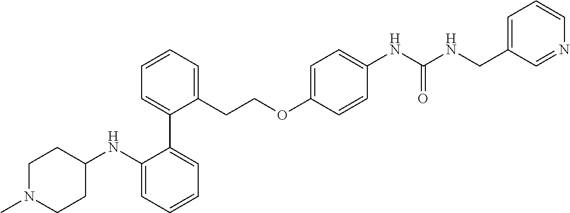 [SMILES: CN1CCC(Nc2ccccc2-c2ccccc2CCOc2ccc(NC(=O)NCc3cccnc3)cc2)CC1] | 1-[4-(2-{2′-[(1- Methylpiperidin- 4-yl)amino] biphenyl-2- yl}ethoxy) phenyl]- 3-(pyridin-3- ylmethyl)urea | LC-MS [M + H]+ 536.3020 | |
| 745 | 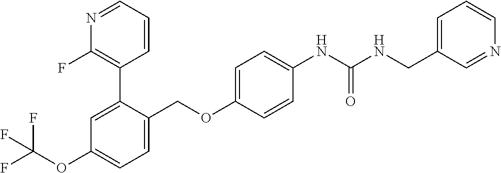 [SMILES: O=C(NCc1cccnc1)Nc1ccc(OCc2ccc(OC(F)(F)F)cc2-c2cccnc2F)cc1] | 1-(4-{[2-(2- Fluoropyridin-3- yl)-4-(trifluoro- methoxy) benzyl]oxy} phenyl)- 3-(pyridin-3- ylmethyl)urea | LC-MS [M + H]+ 513.1546 | |
| 746 | 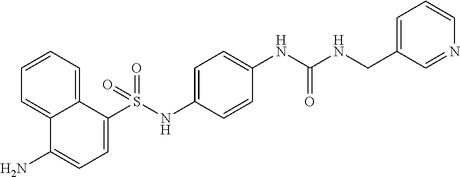 [SMILES: Nc1ccc(S(=O)(=O)Nc2ccc(NC(=O)NCc3cccnc3)cc2)c2ccccc12] | 4-Amino-N-(4- {[(pyridin-3- ylmethyl) carbamoyl] amino}phenyl) naphthalene-1- sulfonamide | LC-MS [M + H]+ 448.1448 | |
| 747 | 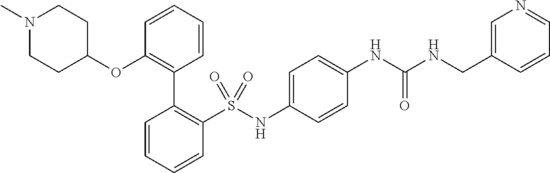 [SMILES: CN1CCC(Oc2ccccc2-c2ccccc2S(=O)(=O)Nc2ccc(NC(=O)NCc3cccnc3)cc2)CC1] | 2′-[(1- Methylpiperidin- 4-yl)oxy]-N-(4- {[(pyridin-3- ylmethyl) carbamoyl] amino}phenyl)bi- phenyl-2- sulfonamide | LC-MS [M + H]+ 572.2312 | |
| 748 | 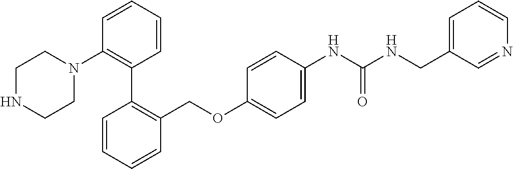 [SMILES: O=C(NCc1cccnc1)Nc1ccc(OCc2ccccc2-c2ccccc2N2CCNCC2)cc1] | 1-(4-{[2′- (Piperazin-1- yl)biphenyl-2- yl]methoxy} phenyl)- 3-(pyridin-3- ylmethyl)urea | LC-MS [M + H]+ 494.2644 | |
| 749 | 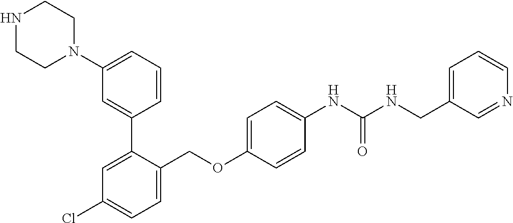 [SMILES: O=C(NCc1cccnc1)Nc1ccc(OCc2ccc(Cl)cc2-c2cccc(N3CCNCC3)c2)cc1] | 1-(4-{[5-Chloro- 3′-(piperazin-1- yl)biphenyl-2- yl]methoxy} phenyl)- 3-(pyridin-3- ylmethyl)urea | LC-MS [M + H]+ 528.2190 | |
| 750 | 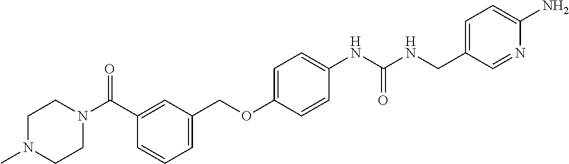 [SMILES: CN1CCN(C(=O)c2cccc(COc3ccc(NC(=O)NCc4ccc(N)nc4)cc3)c2)CC1] | 1-[(6- Aminopyridin-3- yl)methyl]-3-[4- ({3-[(4- methylpiperazin- 1-yl)carbonyl] benzyl} oxy)phenyl]urea | LC-MS [M + H]+ 475.2438 | |
| 751 | 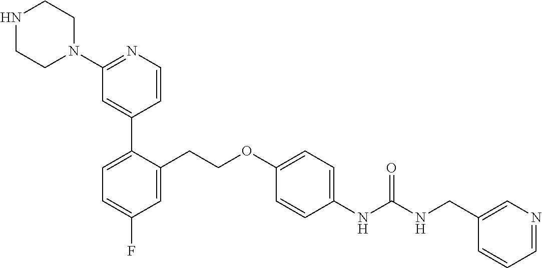 [SMILES: O=C(NCc1cccnc1)Nc1ccc(OCCc2cc(F)ccc2-c2ccnc(N3CCNCC3)c2)cc1] | 1-[4-(2-{5- Fluoro-2-[2- (piperazin-1- yl)pyridin-4- yl]phenyl}ethoxy) phenyl]-3- (pyridin- 3-ylmethyl)urea | LC-MS [M + H]+ 527.2562 | 8.51 (s, 1H), 8.45 (d, 1H), 8.41 (s, 1H), 8.17 (d, 1H), 7.70 (dd, 1H), 7.41-7.13 (m, 6H), 6.79 (s, 1H), 6.75-6.64 (m, 3H), 6.59 (t, 1H), 4.29 (d, 2H), 4.04 (t, 2H), 3.06-2.84 (m, 10H) |
| 752 | 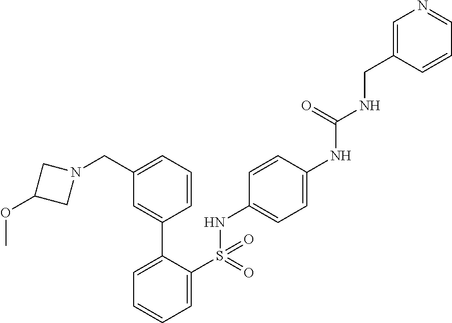 | 5-Fluoro-3′-[(3- methoxyazetidin- 1-yl)methyl]-N- (4-{[(pyridin-3- ylmethyl) carbamoyl] amino}phenyl)bi- phenyl-2- sulfonamide | LC-MS [M + H]+ 576.2085 | |
| 753 | 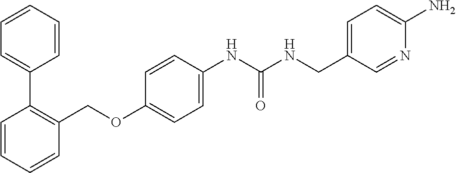 [SMILES: Nc1ccc(CNC(=O)Nc2ccc(OCc3ccccc3-c3ccccc3)cc2)cn1] | 1-[(6- Aminopyridin-3- yl)methyl]-3-[4- (biphenyl-2- ylmethoxy) phenyl] urea | LC-MS [M + H]+ 425.1984 | |
| 754 | 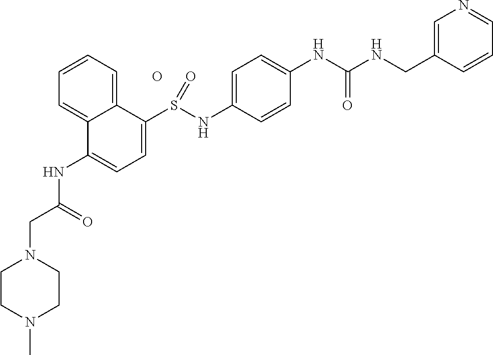 | 2-(4- Methylpiperazin- 1-yl)-N-{4-[(4- {[(pyridin-3- ylmethyl) carbamoyl] amino}phenyl) sulfamoyl] naphthalen- 1-yl}acetamide | LC-MS [M + H]+ 588.2379 | |
| 755 | 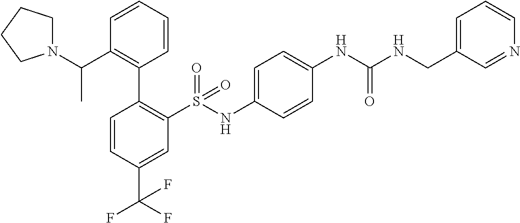 [SMILES: CC(c1ccccc1-c1ccc(C(F)(F)F)cc1S(=O)(=O)Nc1ccc(NC(=O)NCc2cccnc2)cc1)N1CCCC1] | N-(4-{[(Pyridin- 3-ylmethyl) carbamoyl] amino}phenyl)- 2′-[1-(pyrrolidin- 1-yl)ethyl]-4- (trifluoromethyl) biphenyl-2- sulfonamide | LC-MS [M + H]+ 624.2404 | |
| 756 | 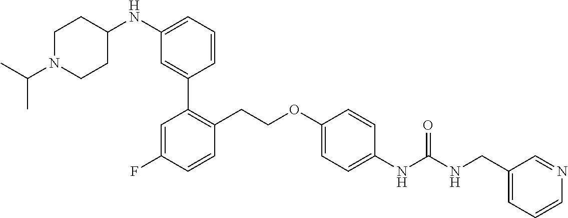 [SMILES: CC(C)N1CCC(Nc2cccc(-c3cc(F)ccc3CCOc3ccc(NC(=O)NCc4cccnc4)cc3)c2)CC1] | 1-{4-[2-(5- Fluoro- 3′-{[1-(propan-2- yl)piperidin-4- yl]amino} biphenyl-2- yl)ethoxy] phenyl}- 3-(pyridin-3- ylmethyl)urea | LC-MS [M + H]+ 582.3313 | 8.79 (bs, 1H), 8.55 (s, 1H), 8.50 (d, 1H), 8.44 (ds, 1H), 7.80 (s, 1H), 7.53-7.43 (m, 2H), 7.24 (d, 2H), 7.21-7.11 (m, 2H), 7.08- 6.96 (m, 2H), 6.72-6.49 (m, 5H), 4.32 (d, 2H), 3.96 (t, 2H), 3.30-3.14 (m, 4H), 3.09- 2.89 (m, 3H), 2.18-2.03 (m, 2H), 1.65-1.46 (m, 2H), 1.17 (d, 6H) |
| 757 | 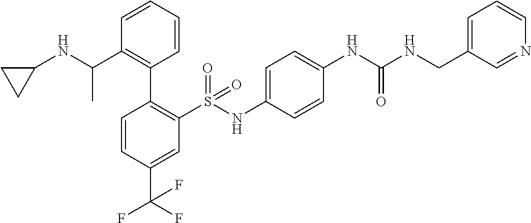 [SMILES: CC(NC1CC1)c1ccccc1-c1ccc(C(F)(F)F)cc1S(=O)(=O)Nc1ccc(NC(=O)NCc2cccnc2)cc1] | 2′-[1- (Cyclopropyl- amino) ethyl]-N-(4- {[(pyridin-3- ylmethyl) carbamoyl] amino}phenyl)- 4- (trifluoromethyl) biphenyl-2- sulfonamide | LC-MS [M + H]+ 610.2066 | |
| 758 | 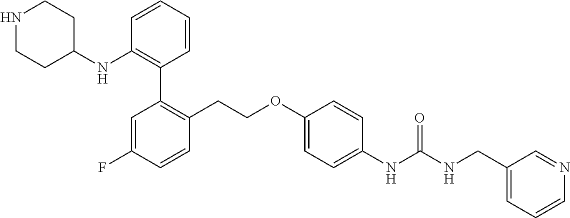 [SMILES: O=C(NCc1cccnc1)Nc1ccc(OCCc2ccc(F)cc2-c2ccccc2NC2CCNCC2)cc1] | 1-(4-{2-[5- Fluoro- 2′-(piperidin-4- ylamino) biphenyl-2- yl]ethoxy} phenyl)- 3-(pyridin-3- ylmethyl)urea | LC-MS [M + H]+ 540.2826 | 8.51 (d, 1H), 8.49 (d, 1H), 8.45 (dd, 1H), 7.69 (dt, 1H), 7.52 (dd, 1H), 7.35 (dd, 1H), 7.30-7.16 (m, 5H), 7.00-6.93 (d, (m, 2H), 6.78 (d, 1H), 6.69 (t, 1H), 6.54 (d, 2H), 4.29 (d, 2H), 3.87 (t, 2H), 3.49 (d, 1H), 2.88-2.64 (m, 4H), 2.37- 2.38 (m, 2H), 1.74 (t, 2H), 1.03 (t, 2H) |
| 759 | 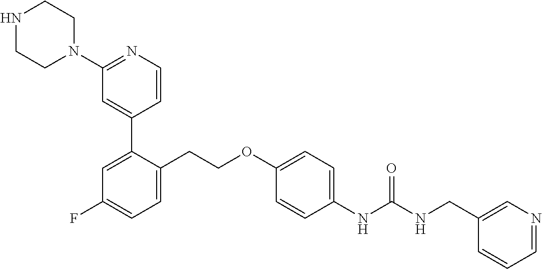 [SMILES: O=C(NCc1cccnc1)Nc1ccc(OCCc2ccc(F)cc2-c2ccnc(N3CCNCC3)c2)cc1] | 1-[4-(2-{4- Fluoro- 2-[2-(piperazin- 1-yl)pyridin-4- yl]phenyl} ethoxy) phenyl]-3- (pyridin- 3-ylmethyl)urea | LC-MS [M + H]+ 527.2578 | 8.51 (s, 1H), 8.45 (d, 1H), 8.40 (s, 1H), 8.19 (d, 1H), 7.69 (d, 1H), 7.52 (dd, 1H), 7.35 (dd, 1H), 7.27-7.21 (m, 3H), 7.08 (d, 1H), 6.83 (s, 1H), 6.74-6.63 (m, 3H), 6.58 (d, 1H), 4.29 (d, 2H), 3.99 (t, 2H), 3.06-2.87 (m, 10H) |
| 760 | 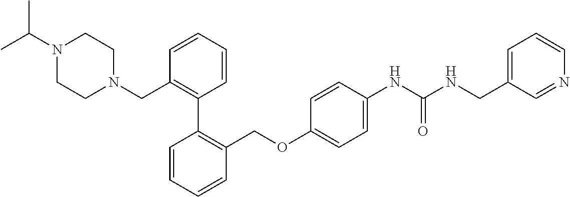 [SMILES: CC(C)N1CCN(Cc2ccccc2-c2ccccc2COc2ccc(NC(=O)NCc3cccnc3)cc2)CC1] | 1-{4-[(2′-{[4- (Propan-2- yl)piperazin-1- yl]methyl} biphenyl-2- yl)methoxy] phenyl}- 3-(pyridin-3- ylmethyl)urea | LC-MS [M + H]+ 550.3192 | |
| 761 | 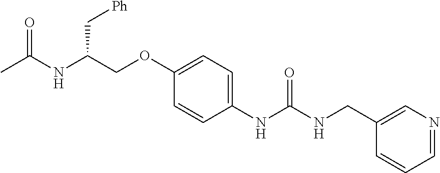 [SMILES: CC(=O)N[C@@H](COc1ccc(NC(=O)NCc2cccnc2)cc1)Cc1ccccc1] | N-[(2R)-1- Phenyl- 3-(4-{[(pyridin- 3-ylmethyl) carbamoyl] amino}phenoxy) propan-2- yl]acetamide | LC-MS [M + H]+ 419.2097 | 8.53 (s,1H), 8.49-8.42 (m, 2H), 8.04 (d, 1H), 7.71 (dd, 1H), 7.40-7.08 (m, 10H), 6.98 (bs, 1H), 6.83 (d, 2H), 6.62 (t, 1H, 4.30 (d, 2H), 4.19 (q, 1H), 3.81 (d, 2H), 1.79 (s, 3H), 1.27 (d, 2H) |
| 762 | 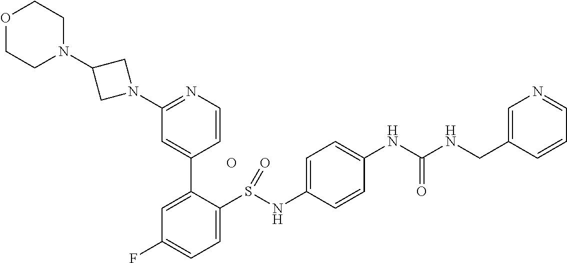 | 4-Fluoro-2-{2-[3- (morpholin-4- yl)azetidin-1- yl]pyridin-4-yl}- N-(4-{[(pyridin- 3-ylmethyl) carbamoyl] amino}phenyl) benzenesulfon- amide | LC-MS [M + H]+ 618.2280 | |
| 763 | 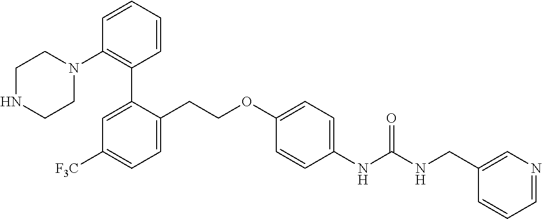 [SMILES: O=C(NCc1cccnc1)Nc1ccc(OCCc2ccc(C(F)(F)F)cc2-c2ccccc2N2CCNCC2)cc1] | 1-(4-{2-[2′- (Piperazin-1-yl)- 5- (trifluoromethyl) biphenyl-2- yl]ethoxy} phenyl)- 3-(pyridin-3- ylmethyl)urea | 577.2612 (M + H) | 8.64-8.52 (m, 5H), 7.97 (bs, 1H), 7,72 (s, 2H), 7,61 (s, 2H), 7.55 (dd, 1H), 7.30-7.19 (m, 6H), 6.70 (t, 1H), 6.63 (d, 2H), 4.35 (d, 2H), 4.05-3.94 (m, 2H), 3.13- 2.71 (m, 10H) |
| 764 | 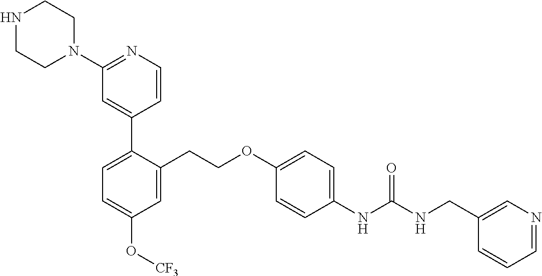 [SMILES: O=C(NCc1cccnc1)Nc1ccc(OCCc2cc(OC(F)(F)F)ccc2-c2ccnc(N3CCNCC3)c2)cc1] | 1-[4-(2-{2-[2- (Piperazin-1- yl)pyridin-4-yl]- 5-(trifluoro- methoxy) phenyl}ethoxy) phenyl]-3- (pyridin- 3-ylmethyl)urea | LC-MS [M + H]+ 593.2467 | 8.51 (s, 1H), 8.45 (d, 1H), 8.41 (s, 1H), 8.19 (d, 1H), 7.70 (dd, 1H), 7.52 (s, 1H), 7.40-7.26 (m, 5H), 7.24 (d, 2H), 6.68 (d, 2H), 6.64-6.54 (m, 1H), 4.29 (d, 2H), 4.04 (t, 2H), 3.09-2.84 (m, 10H) |
| 765 | 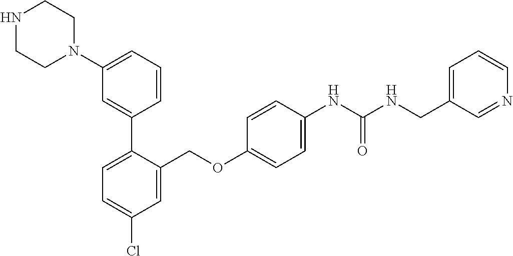 [SMILES: O=C(NCc1cccnc1)Nc1ccc(OCc2cc(Cl)ccc2-c2cccc(N3CCNCC3)c2)cc1] | 1-(4-{[4-Chloro- 3′-(piperazin-1- yl)biphenyl-2- yl]methoxy} phenyl)- 3-(pyridin-3- ylmethyl)urea | LC-MS [M + H]+ 528.2170 | 8.51 (s, 1H), 8.47-8.41 (m, 2H), 7.70 (dt, 1H), 7.64 (d, 1H), 7.63-7.54 (d, 1H), 7.49 (dd, 1H), 7.40- 7.23 (m, 6H), 6.98-6.89 (m, 2H), 6.84-6.76 (m, 3H), 6.60 (d, 2H), 4.86 (s, 2H), 4.30 (d, 2H), 3.03-2.93 (m, 4H), 2.80- 2.72 (m, 4H) |
| 766 | 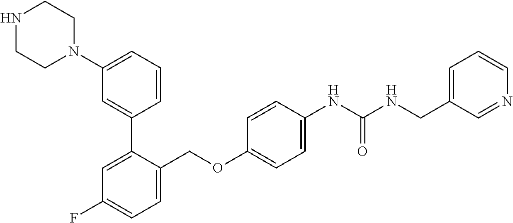 [SMILES: O=C(NCc1cccnc1)Nc1ccc(OCc2ccc(F)cc2-c2cccc(N3CCNCC3)c2)cc1] | 1-(4-{[5-Fluoro- 3′-(piperazin-1- yl)biphenyl-2- yl]methoxy} phenyl)- 3-(pyridin-3- ylmethyl)urea | LC-MS [M + H]+ 512.2462 | 8.51 (s, 1H), 8.47-8.42 (m, 2H), 7.70 (d, 1H), 7.64 (dd, 1H), 7.39-7.16 (m, 6H), 7.02- 6.95 (m, 2H), 6.88 (d, 1H), 6.80 (d, 2H), 6.61 (t, 1H), 4.82 (s, 2H), 4.30 (d, 2H), 3.20-3.09 (m, 4H), 3.02-2.88 (m, 4H) |
| 767 | 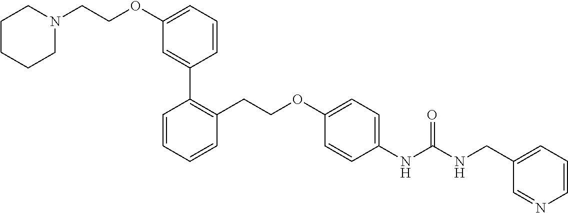 [SMILES: O=C(NCc1cccnc1)Nc1ccc(OCCc2ccccc2-c2cccc(OCCN3CCCCC3)c2)cc1] | 1-[4-(2-{3′-[2- (Piperidin-1- yl)ethoxy] biphenyl-2- yl}ethoxy) phenyl]- 3-(pyridin-3- ylmethyl)urea | LC-MS [M + H]+ 551.3017 | |
| 768 | 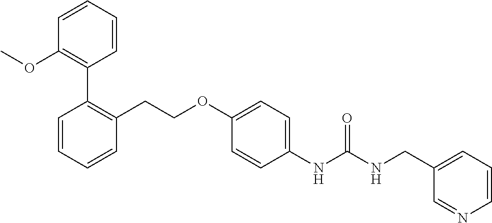 [SMILES: COc1ccccc1-c1ccccc1CCOc1ccc(NC(=O)NCc2cccnc2)cc1] | 1-{4-[2-(2′- Methoxy- biphenyl-2- yl)ethoxy] phenyl}- 3-(pyridin-3- ylmethyl)urea | LC-MS [M + Na]+ 476.1932 | |
| 769 | 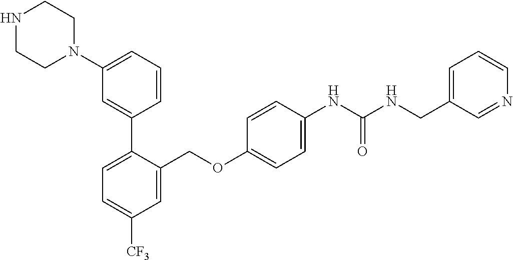 [SMILES: O=C(NCc1cccnc1)Nc1ccc(OCc2cc(C(F)(F)F)ccc2-c2cccc(N3CCNCC3)c2)cc1] | 1-(4-{[3′- (Piperazin-1-yl)- 4- (trifluoromethyl) biphenyl-2- yl]methoxy} phenyl)- 3-(pyridin-3- ylmethyl)urea | LC-MS [M + H]+ 562.2433 | 8.51 (s, 1H), 8.47-8.42 (m, 2H), 7.95 (s, 1H), 7.79 (d, 1H), 7.70 (dt, 1H), 7.58 (d, 1H), 7.35 (dd, 1H), 7.32-7.26 (m, 3H), 6.99- 6.94 (m, 2H), 6.85-6.78 (m, 3H), 6.60 (t, 1H), 4.94 (s, 2H), 4.30 (d, 2H), 3.01-2.94 (m, 4H), 2.79- 2.70 (m, 4H) |
| 770 | 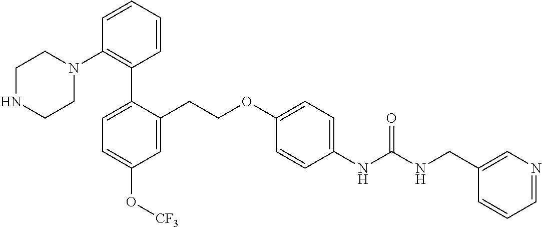 [SMILES: O=C(NCc1cccnc1)Nc1ccc(OCCc2cc(OC(F)(F)F)ccc2-c2ccccc2N2CCNCC2)cc1] | 1-(4-{2-[2′- (Piperazin-1-yl)- 4-(trifluoro- methoxy) biphenyl-2- yl]ethoxy} phenyl)- 3-(pyridin-3- ylmethyl)urea | LC-MS [M + H]+ 592.2557 | 8.55-8.41 (m, 5H), 7.71 (d, 1H), 7.50-7.15 (m, 10H), 6.61 (d, 2H), 4.29 (d, 2H), 3.98-3.90 (m, 2H), 3.06- 2.74 (m, 10H) |
| 771 | 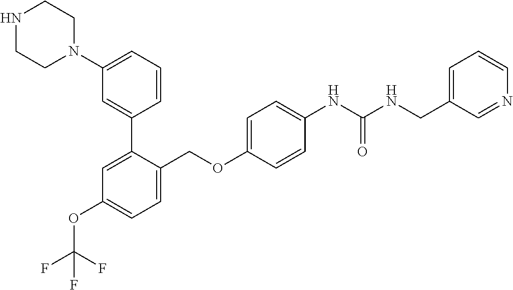 [SMILES: O=C(NCc1cccnc1)Nc1ccc(OCc2ccc(OC(F)(F)F)cc2-c2cccc(N3CCNCC3)c2)cc1] | 1-(4-{[3′- (Piperazin-1-yl)- 5-(trifluoro- methoxy) biphenyl-2- yl]methoxy} phenyl)- 3-(pyridin-3- ylmethyl)urea | LC-MS [M + H]+ 578.2391 | 8.54-8.49 (m, 2H), 8.45 (dd, 1H), 7.73 (d, 1H), 7.70 (dt, 1H), 7.44 (d, 1H), 7.40-7.29 (m, 4H), 7.27 (d, 2H), 7.06- 7.00 (m, 2H), 6.92 (d, 1H), 6.79 (d, 2H), 6.68 (t, 1H), 4.88 (s, 2H), 4.30 (d, 2H), 3.32-3.25 (m, 4H), 3.15-3.08 (m, 4H) |
| 772 | 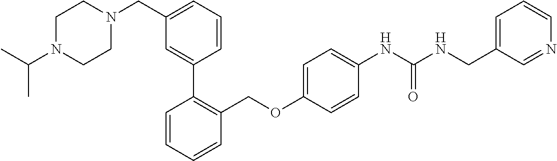 [SMILES: CC(C)N1CCN(Cc2cccc(-c3ccccc3COc3ccc(NC(=O)NCc4cccnc4)cc3)c2)CC1] | 1-{4-[(3′-{[4- (Propan-2- yl)piperazin-1- yl]methyl} biphenyl-2- yl)methoxy] phenyl}- 3-(pyridin-3- ylmethyl)urea | LC-MS [M + H]+ 550.3191 | |
| 773 | 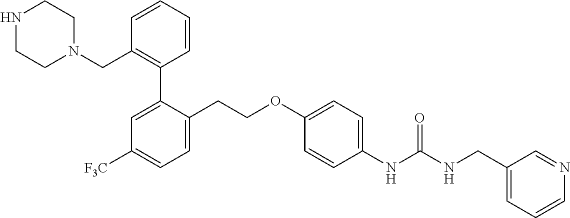 [SMILES: O=C(NCc1cccnc1)Nc1ccc(OCCc2ccc(C(F)(F)F)cc2-c2ccccc2CN2CCNCC2)cc1] | 1-(4-{2-[2′- (Piperazin-1- ylmethyl)-5- (trifluoromethyl) biphenyl-2- yl]ethoxy} phenyl)- 3-(pyridin-3- ylmethyl)urea | LC-MS [M + H]+ 590.2831 | 9.34 (bs, 2H), 8.79-8.57 (m, 5H), 8.33 (bs, 1H), 8.21 (d, 1H), 8.01 (d, 1H), 7.84-7.39 (m, 7H), 7.28 (d, 1H), 7.22 (d, 2H), 6.84 (t, 1H), 6.64 (d, 2H), 4.40 (d, 2H), 4.11 (q, 2H), 3.98 (q, 2H), 3.29-2.80 (m, 10H) |
| 774 | 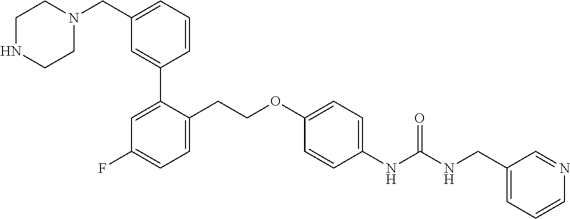 [SMILES: O=C(NCc1cccnc1)Nc1ccc(OCCc2ccc(F)cc2-c2cccc(CN3CCNCC3)c2)cc1] | 1-(4-{2-[5- Fluoro- 3′-(piperazin-1- ylmethyl) biphenyl-2- yl]ethoxy} phenyl)- 3-(pyridin-3- ylmethyl)urea | LC-MS [M + H]+ 540.2893 | 9.35 (bs, 1H), 9.25 (bs, 1H), 8.78-8.68 (m, 2H), 8.66 (d, 1H), 8.26 (d, 1H), 8.03 (d, 1H), 7.86 (d, 1H), 7.69-7.39 (m, 6H), 7.27- 7.19 (m, 3H), 7.12 (dd, 1H), 6.88 (t, 1H), 6.68 (d, 2H), 4.41 (d, 2H), 4.12 (q, 2H), 3.97 (t, 2H), 3.43-3.04 (m, 8H), 2.96 (t, 2H) |
| 775 | 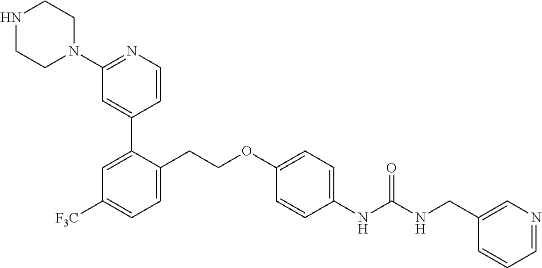 [SMILES: O=C(NCc1cccnc1)Nc1ccc(OCCc2ccc(C(F)(F)F)cc2-c2ccnc(N3CCNCC3)c2)cc1] | 1-[4-(2-{2-[2- (Piperazin-1- yl)pyridin-4-yl]- 4- (trifluoromethyl) phenyl}ethoxy) phenyl]- 3-(pyridin-3- ylmethyl)urea | LC-MS [M + H]+ 577.2538 | 8.51 (s, 1H), 8.45 (d, 1H), 8.41 (s, 1H), 8.21 (d, 1H), 7.75 (d, 1H), 7.53 (s, 1H), 7.41-7.20 (m, 4H), 6.91 (s, 1H), 6.79-6.54 (m, 4H), 4.29 (d, 2H), 4.05 (t, 2H), 3.09-2.88 (m, 10H) |
| 776 | 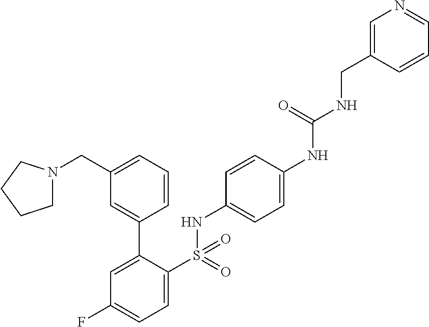 [SMILES: O=C(NCc1cccnc1)Nc1ccc(NS(=O)(=O)c2ccc(F)cc2-c2cccc(CN3CCCC3)c2)cc1] | 5-Fluoro-N-(4- {[(pyridin-3- ylmethyl) carbamoyl] amino}phenyl)- 3′-(pyrrolidin-1- ylmethyl) biphenyl- 2-sulfonamide | LC-MS [M + H]+ 560.2141 | |
| 777 | 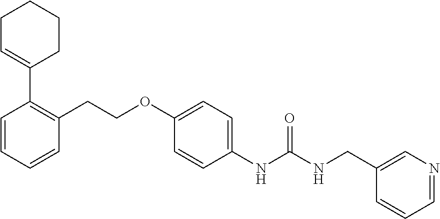 [SMILES: O=C(NCc1cccnc1)Nc1ccc(OCCc2ccccc2C2=CCCCC2)cc1] | 1-(4-{2-[2- (Cyclohex-1-en- 1-yl)phenyl] ethoxy} phenyl)-3- (pyridin- 3-ylmethyl)urea | LC-MS [M + H]+ 428.9232 | 8.70 (s, 1H), 8.67 )d, 1H), 8.56 (s, 1H), 8.16 (d, 1H), 7.76 (dd, 1H) 7.35-7.14 (m, 5H), 7.06 (dd, 1H), 6.82 (d, 1H), 5.54 (p, 1H), 4.39 (d, 2H), 4.07 (t, 2H), 2.98 (t, 2H), 2.21-2.07 (m, 4H), 1.75- 1.58 (m, 4H) |
| 778 | 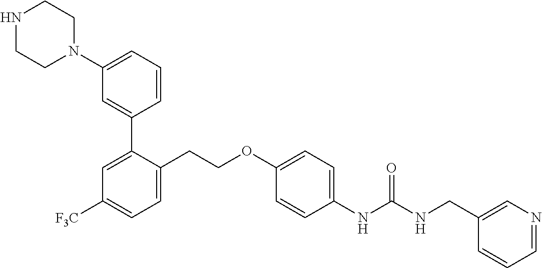 [SMILES: O=C(NCc1cccnc1)Nc1ccc(OCCc2ccc(C(F)(F)F)cc2-c2cccc(N3CCNCC3)c2)cc1] | 1-(4-{2-[3′- (Piperazin-1-yl)- 5- (trifluoromethyl) biphenyl-2- yl]ethoxy} phenyl)- 3-(pyridin-3- ylmethyl)urea | LC-MS [M + H]+ 576.2622 | 8.87 (bs, 2H), 8.74-8.62 (m, 3H), 8.16 (d, 1H), 7.76 (t, 1H), 7.71 (s, 2H), 7.49 (s, 1H), 7.37 (t, 1H), 7.23 (d, 2H), 7.06 (dd, 1H), 7.02 (s, 1H), 6.88 (d, 1H), 6.83 (t, 1H), 6.65 (d, 2H), 4.39 (d, 2H), 4.02 (t, 2H), 3.45-3.37 (m, 4H), 3.25- 3.16 (m, 4H), 3.04 (t, 2H) |
| 779 | 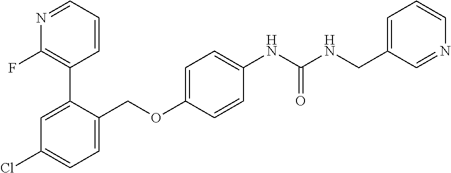 [SMILES: O=C(NCc1cccnc1)Nc1ccc(OCc2ccc(Cl)cc2-c2cccnc2F)cc1] | 1-(4-{[4-Chloro- 2-(2- fluoropyridin- 3- yl)benzyl]oxy} phenyl)-3- (pyridin- 3-ylmethyl)urea | LC-MS [M + H]+ 463.1318 | |
| 780 | 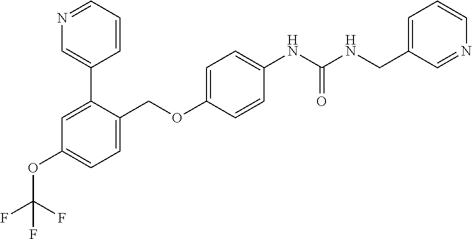 [SMILES: O=C(NCc1cccnc1)Nc1ccc(OCc2ccc(OC(F)(F)F)cc2-c2cccnc2)cc1] | 1-(Pyridin-3- ylmethyl)-3-(4- {[2- (pyridin-3-yl)-4- (trifluoro- methoxy) benzyl]oxy} phenyl) urea | LC-MS [M + H]+ 495.1640 | |
| 781 | 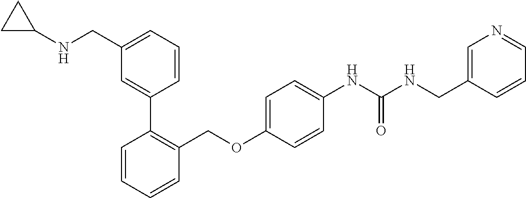 [SMILES: O=C(NCc1cccnc1)Nc1ccc(OCc2ccccc2-c2cccc(CNC3CC3)c2)cc1] | 1-{4-[({3′- [(Cyclopropyl- amino) methyl]biphenyl- 2- yl}oxy)methyl] phenyl}-3- (pyridin- 3-ylmethyl)urea | LC-MS [M + H]+ 479.2697 | |
| 782 | 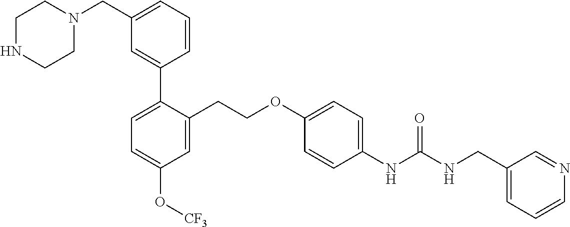 [SMILES: O=C(NCc1cccnc1)Nc1ccc(OCCc2cc(OC(F)(F)F)ccc2-c2cccc(CN3CCNCC3)c2)cc1] | 1-(4-{2-[3′- (Piperazin-1- ylmethyl)-4- (trifluoro- methoxy) biphenyl-2- yl]ethoxy} phenyl)- 3-(pyridin-3- ylmethyl)urea | LC-MS [M + H]+ 606.2825 | 9.07 (bs, 1H), 8.72-8.48 (m, 4H), 7.91 (d, 1H), 7.62-7.28 (m, 8H), 7.23 (d, 2H), 6.73- 6.64 (m, 3H), 4.34 (d, 2H), 4.10 (q, 2H), 4.00 (t, 2H), 3.31-3.08 (m, 8H), 3.00 (t, 2H) |
| 783 | 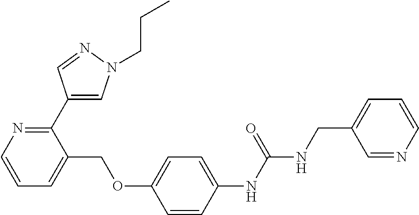 [SMILES: CCCn1cc(-c2ncccc2COc2ccc(NC(=O)NCc3cccnc3)cc2)cn1] | 1-(4-{[2-(1- Propyl- 1H-pyrazol-4- yl)pyridin-3- yl]methoxy} phenyl)- 3-(pyridin-3- ylmethyl)urea | LC-MS [M + H]+ 443.2195 | |
| 784 | 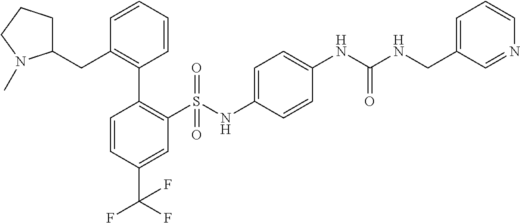 [SMILES: CN1CCCC1Cc1ccccc1-c1ccc(C(F)(F)F)cc1S(=O)(=O)Nc1ccc(NC(=O)NCc2cccnc2)cc1] | 2′-[(2- Methyl- pyrrolidin- yl)methyl]-N-(4- 1-{[(pyridin-3- ylmethyl) carbamoyl] amino}phenyl)- 4- (trifluoromethyl) biphenyl-2- sulfonamide | LC-MS [M + H]+ 624.2268 | |
| 785 | 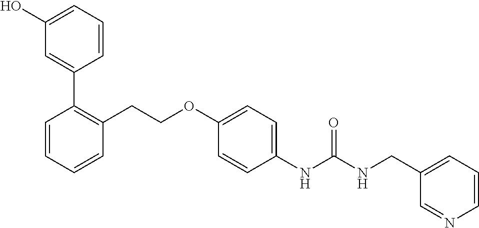 [SMILES: O=C(NCc1cccnc1)Nc1ccc(OCCc2ccccc2-c2cccc(O)c2)cc1] | 1-{4-[2-(3′- Hydroxy- biphenyl-2- yl)ethoxy] phenyl}- 3-(pyridin-3- ylmethyl)urea | LC-MS [M + H]+ 440.1954 | |
| 786 | 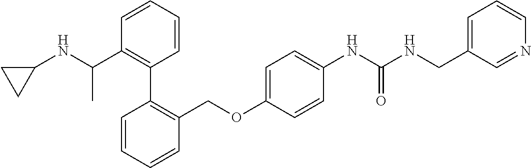 [SMILES: CC(NC1CC1)c1ccccc1-c1ccccc1COc1ccc(NC(=O)NCc2cccnc2)cc1] | 1-[4-({2′-[1- (Cyclopropyl- amino) ethyl]biphenyl- 2-yl}methoxy) phenyl]- 3-(pyridin-3- ylmethyl)urea | LC-MS [M + H]+ 493.2609 | |

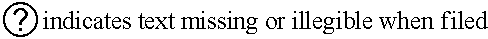

TABLE 6

| No. | Structure | IUPAC Name | LC-MS | 1H NMR |
|---|---|---|---|---|
| 787 | 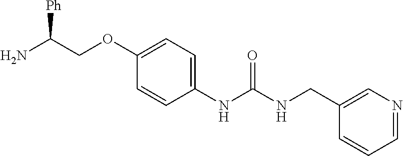 [SMILES: N[C@H](COc1ccc(NC(=O)NCc2cccnc2)cc1)c1ccccc1] | 1-{4-[(2S)-2- Amino-2-phenyl- ethoxy]phenyl}- 3-(pyridin-3- ylmethyl)urea | LC-MS [M + H]+ 363.1816 | |
| 788 | 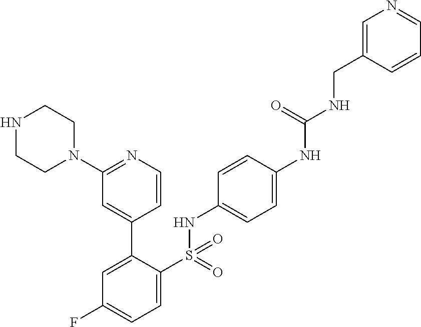 [SMILES: O=C(NCc1cccnc1)Nc1ccc(NS(=O)(=O)c2ccc(F)cc2-c2ccnc(N3CCNCC3)c2)cc1] | 4-Fluoro-2-[2- (piperazin-1- yl)pyridin-4-yl]-N- (4-{[(pyridin-3- ylmethyl)carbamoyl] amino}phenyl) benzenesulfonamide | LC-MS [M + H]+ 562.2049 | |
| 789 | 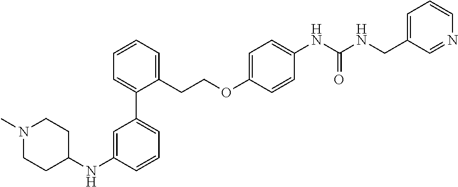 [SMILES: CN1CCC(Nc2cccc(-c3ccccc3CCOc3ccc(NC(=O)NCc4cccnc4)cc3)c2)CC1] | 1-[4-(2-{3′-[(1- Methylpiperidin-4- yl)amino]biphenyl- 2- yl}ethoxy)phenyl]- 3-(pyridin-3- ylmethyl)urea | LC-MS [M + H]+ 536.3189 | |
| 790 | 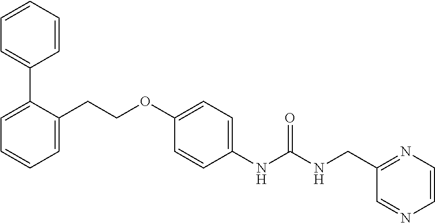 [SMILES: O=C(NCc1cnccn1)Nc1ccc(OCCc2ccccc2-c2ccccc2)cc1] | 1-{4-[2-(Biphenyl- 2- yl)ethoxy]phenyl}- 3-(pyrazin-2- ylmethyl)urea | LC-MS [M + H]+ 425.1980 | |
| 791 | 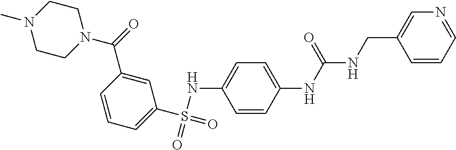 [SMILES: CN1CCN(C(=O)c2cccc(S(=O)(=O)Nc3ccc(NC(=O)NCc4cccnc4)cc3)c2)CC1] | 3-[(4- Methylpiperazin-1- yl)carbonyl]-N-(4- {[(pyridin-3- ylmethyl)carbamoyl] amino}phenyl) benzenesulfonamide | LC-MS [M + H]+ 509.1975 | |
| 792 | 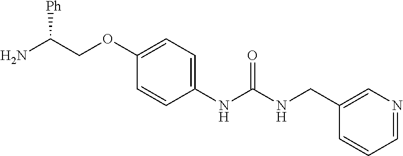 [SMILES: N[C@@H](COc1ccc(NC(=O)NCc2cccnc2)cc1)c1ccccc1] | 1-{4-[(2R)-2- Amino-2-phenyl- ethoxy]phenyl}-3- (pyridin-3- ylmethyl)urea | LC-MS [M + H]+ 363.1829 | |
| 793 | 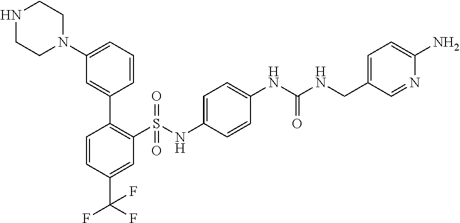 [SMILES: Nc1ccc(CNC(=O)Nc2ccc(NS(=O)(=O)c3cc(C(F)(F)F)ccc3-c3cccc(N4CCNCC4)c3)cc2)cn1] | N-[4-({[(6- Aminopyridin-3- yl)methyl] carbamoyl}amino) phenyl]-3′- (piperazin-1-yl)-4- (trifluoromethyl) biphenyl-2- sulfonamide | LC-MS [M + H]+ 626.2186 | |
| 794 | 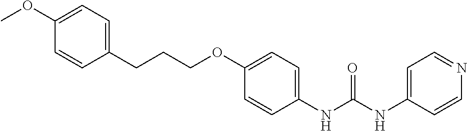 [SMILES: COc1ccc(CCCOc2ccc(NC(=O)Nc3ccncc3)cc2)cc1] | 1-{4-[3-(4- Methoxyphenyl) propoxy]phenyl}- 3-pyridin-4-ylurea | LC-MS [M + H]+ 378.1819 | |
| 795 | 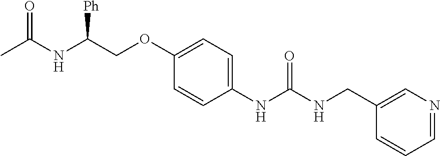 [SMILES: CC(=O)N[C@H](COc1ccc(NC(=O)NCc2cccnc2)cc1)c1ccccc1] | N-[(1S)-1-Phenyl- 2-(4-{[(pyridin-3- ylmethyl)carbamoyl] amino}phenoxy) ethyl]acetamide | LC-MS [M + H]+ 405.1921 | |
| 796 | 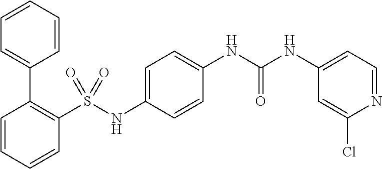 [SMILES: O=C(Nc1ccc(NS(=O)(=O)c2ccccc2-c2ccccc2)cc1)Nc1ccnc(Cl)c1] | N-(4-{[(2- Chloropyridin-4- yl)carbamoyl]amino} phenyl)biphenyl-2- sulfonamide | LC-MS [M + H]+ 479.0931 | |
| 797 | 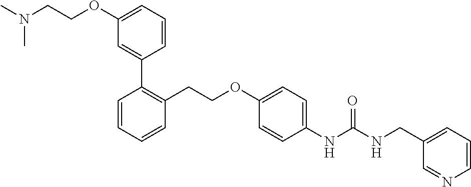 [SMILES: CN(C)CCOc1cccc(-c2ccccc2CCOc2ccc(NC(=O)NCc3cccnc3)cc2)c1] | 1-[4-(2-{3′-[2- (Dimethylamino) ethoxy]biphenyl- 2-yl}ethoxy)phenyl]- 3-(pyridin-3- ylmethyl)urea | LC-MS [M + H]+ 511.2698 | |
| 798 | 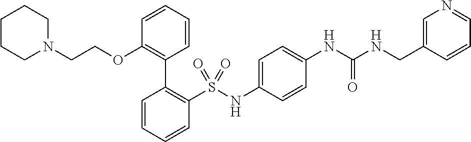 [SMILES: O=C(NCc1cccnc1)Nc1ccc(NS(=O)(=O)c2ccccc2-c2ccccc2OCCN2CCCCC2)cc1] | 2′-[2-(Piperidin-1- yl)ethoxy]-N-(4- {[(pyridin-3- ylmethyl)carbamoyl] amino}phenyl) biphenyl-2- sulfonamide | LC-MS [M + H]+ 586.2468 | |
| 799 | 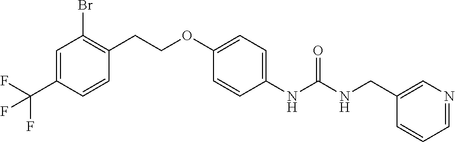 [SMILES: O=C(NCc1cccnc1)Nc1ccc(OCCc2ccc(C(F)(F)F)cc2Br)cc1] | 1-(4-{2-[2-Bromo- 4-(trifluoromethyl) phenyl]ethoxy} phenyl)-3-(pyridin- 3-ylmethyl)urea | LC-MS [M + H]+ 494.0684 | |
| 800 | 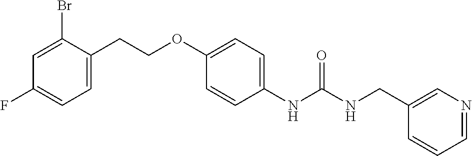 [SMILES: O=C(NCc1cccnc1)Nc1ccc(OCCc2ccc(F)cc2Br)cc1] | 1-{4-[2-(2-Bromo- 4-(fluorophenyl) ethoxy]phenyl}-3- (pyridin-3- ylmethyl)urea | LC-MS [M + H]+ 444.0739 | |
| 801 | 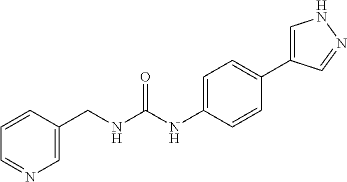 [SMILES: O=C(NCc1cccnc1)Nc1ccc(-c2cn[nH]c2)cc1] | 1-[4-(1H-Pyrazol- 4-yl)phenyl]-3- (pyridin-3- ylmethyl)urea | LC-MS [M + H]+ 294.1349 | |
| 802 | 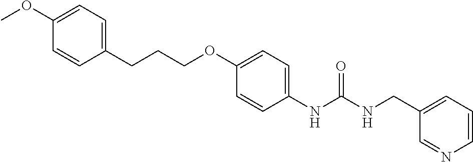 [SMILES: COc1ccc(CCCOc2ccc(NC(=O)NCc3cccnc3)cc2)cc1] | 1-{4-[3-(4- Methoxyphenyl) propoxy]phenyl}-3- (pyridin-3- ylmethyl)urea | LC-MS [M + H]+ 392.1940 | |
| 803 | 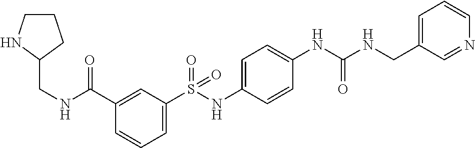 [SMILES: O=C(NCc1cccnc1)Nc1ccc(NS(=O)(=O)c2cccc(C(=O)NCC3CCCN3)c2)cc1] | 3-[(4-{[(Pyridin-3- ylmethyl)carbamoyl] amino}phenyl) sulfamoyl]-N- (pyrrolidin-2- ylmethyl)benzamide | LC-MS [M + H]+ 509.1954 | |
| 804 | 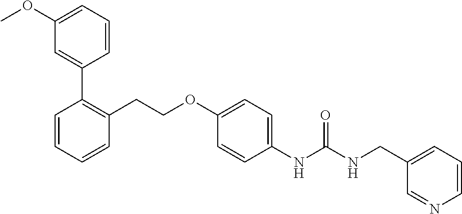 [SMILES: COc1cccc(-c2ccccc2CCOc2ccc(NC(=O)NCc3cccnc3)cc2)c1] | 1-{4-[2-(3′- Methoxybiphenyl- 2- yl)ethoxy]phenyl}- 3-(pyridin-3- ylmethyl)urea | LC-MS [M + H]+ 454.2112 | |
| 805 | 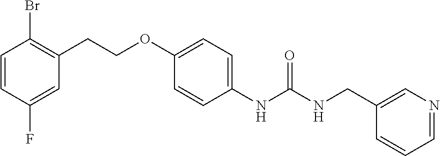 [SMILES: O=C(NCc1cccnc1)Nc1ccc(OCCc2cc(F)ccc2Br)cc1] | 1-{4-[2-(2-Bromo- 5- fluorophenyl)ethoxy] phenyl}-3-(pyridin- 3-ylmethyl)urea | LC-MS [M + H]+ 444.0729 | |
| 806 | 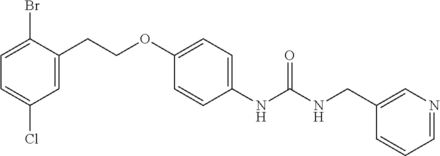 [SMILES: O=C(NCc1cccnc1)Nc1ccc(OCCc2cc(Cl)ccc2Br)cc1] | 1-{4-[2-(2-Bromo- 5- chlorophenyl)ethoxy] phenyl}-3-(pyridin- 3-ylmethyl)urea | LC-MS [M + H]+ 460.0427 | |
| 807 | 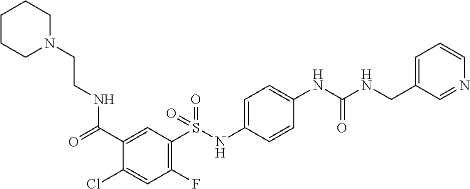 [SMILES: O=C(NCc1cccnc1)Nc1ccc(NS(=O)(=O)c2cc(C(=O)NCCN3CCCCC3)c(Cl)cc2F)cc1] | 2-Chloro-4-fluoro- N-[2-(piperidin-1- yl)ethyl]-5-[(4- {[(pyridin-3- ylmethyl)carbamoyl] amino}phenyl) sulfamoyl]benzamide | LC-MS [M + H]+ 589.1802 | |
| 808 | 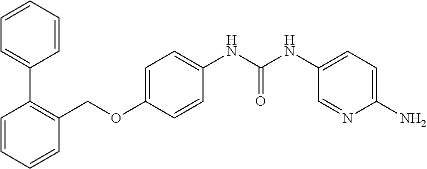 [SMILES: Nc1ccc(NC(=O)Nc2ccc(OCc3ccccc3-c3ccccc3)cc2)cn1] | 1-(6-Aminopyridin- 3-yl)-3-[4- (biphenyl-2- ylmethoxy)phenyl] urea | LC-MS [M + H]+ 411.1789 | |
| 809 | 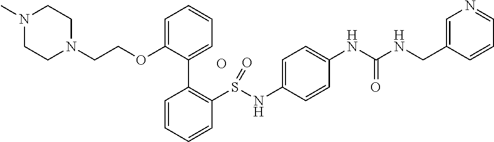 | 2′-[2-(4- Methylpiperazin-1- yl)ethoxy]-N-(4- {[(pyridin-3- ylmethyl)carbamoyl] amino}phenyl) biphenyl-2- sulfonamide | LC-MS [M + H]+ 601.2523 | |
| 810 | 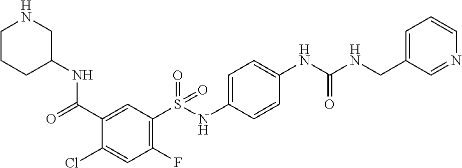 [SMILES: O=C(NCc1cccnc1)Nc1ccc(NS(=O)(=O)c2cc(C(=O)NC3CCCNC3)c(Cl)cc2F)cc1] | 2-Chloro-4-fluoro- N-(piperidin-3-yl)- 5-[(4-{[(pyridin-3- ylmethyl)carbamoyl] amino}phenyl) sulfamoyl]benzamide | LC-MS [M + H]+ 561.1473 | |
| 811 | 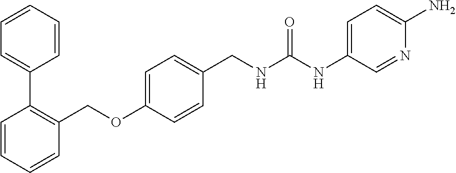 [SMILES: Nc1ccc(NC(=O)NCc2ccc(OCc3ccccc3-c3ccccc3)cc2)cn1] | 1-(6-Aminopyridin- 3-yl)-3-[4- (biphenyl-2- ylmethoxy)benzyl] urea | LC-MS [M + H]+ 425.1980 | |
| 812 | 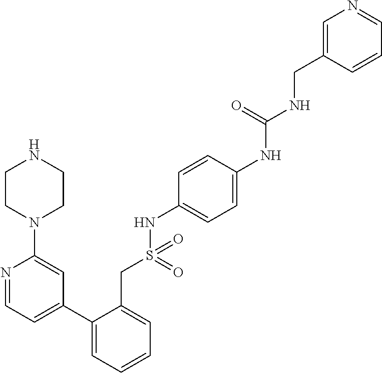 [SMILES: O=C(NCc1cccnc1)Nc1ccc(NS(=O)(=O)Cc2ccccc2-c2ccnc(N3CCNCC3)c2)cc1] | 1-{2-[2-(Piperazin- 1-yl)pyridin-4- yl]phenyl}-N-(4- {[(pyridin-3- ylmethyl)carbamoyl] amino}phenyl) methanesulfonamide | LC-MS [M + H]+ 558.2297 | |
| 813 | 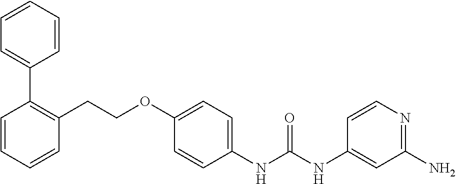 [SMILES: Nc1cc(NC(=O)Nc2ccc(OCCc3ccccc3-c3ccccc3)cc2)ccn1] | 1-(2-Aminopyridin- 4-yl)-3-{4-[2- (biphenyl-2- yl)ethoxy]phenyl} urea | LC-MS [M + H]+ 425.1954 | 10.19 (s, 1H), 9.14 (s, 1H), 7.82 (s, 1H), 7.77 (d, 1H), 7.50-7.27 (m, 11H), 7.21 (d, 1H), 6.74 (d, 2H), 6.55 (s, 1H), 6.48 (bs, 1H), 6.31 (s, 1H), 4.00 (t, 2H), 2.98 (t, 2H) |
| 814 | 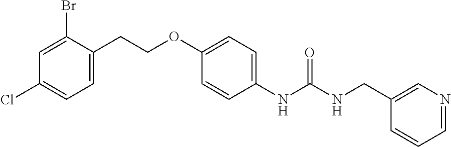 [SMILES: O=C(NCc1cccnc1)Nc1ccc(OCCc2ccc(Cl)cc2Br)cc1] | 1-{4-[2-(2-Bromo- 4- chlorophenyl)ethoxy] phenyl}-3- (pyridin-3- ylmethyl)urea | LC-MS [M + H]+ 460.0414 | |
| 815 | 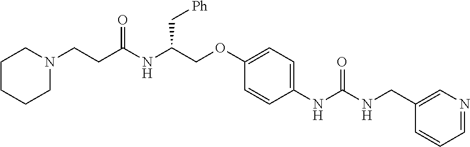 [SMILES: O=C(CCN1CCCCC1)N[C@@H](COc1ccc(NC(=O)NCc2cccnc2)cc1)Cc1ccccc1] | N-[(2R)-1-Phenyl- 3-(4-{[(pyridin-3- ylmethyl)carbamoyl] amino}phenoxy) propan-2-yl]-3- (piperidin-1- yl)propanamide | LC-MS [M + H]+ 516.2991 | |
| 816 | 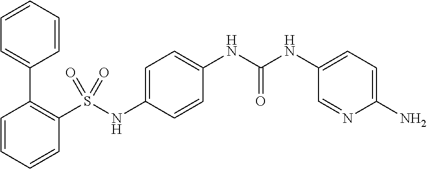 [SMILES: Nc1ccc(NC(=O)Nc2ccc(NS(=O)(=O)c3ccccc3-c3ccccc3)cc2)cn1] | N-(4-{[(6- Aminopyridin-3- yl)carbamoyl)amino} phenyl)biphenyl- 2-sulfonamide | LC-MS [M + H]+ 460.1408 | |
| 817 | 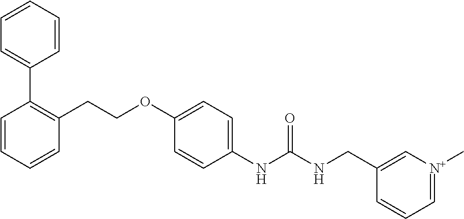 [SMILES: C[n+]1cccc(CNC(=O)Nc2ccc(OCCc3ccccc3-c3ccccc3)cc2)c1] | 3-{[({4-[2- (Biphenyl-2- yl)ethoxy]phenyl} carbamoyl)amino] methyl}-1- methylpyridinium | LC-MS [M + H]+ 439.2241 | |
| 818 | 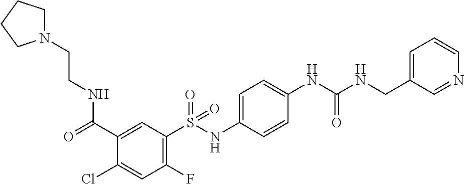 [SMILES: O=C(NCc1cccnc1)Nc1ccc(NS(=O)(=O)c2cc(C(=O)NCCN3CCCC3)c(Cl)cc2F)cc1] | 2-Chloro-4-fluoro- 5-[(4-{[(pyridin-3- ylmethyl)carbamoyl] amino}phenyl) sulfamoyl]-N-[2- (pyrrolidin-1- yl)ethyl]benzamide | LC-MS [M + H]+ 575.1649 | |
| 819 | 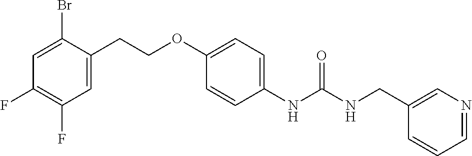 [SMILES: O=C(NCc1cccnc1)Nc1ccc(OCCc2cc(F)c(F)cc2Br)cc1] | 1-{4-[2-(2-Bromo- 4,5-difluoro- phenyl)ethoxy] phenyl}-3-(pyridin- 3-ylmethyl)urea | LC-MS [M + H]+ 462.0633 | |
| 820 | 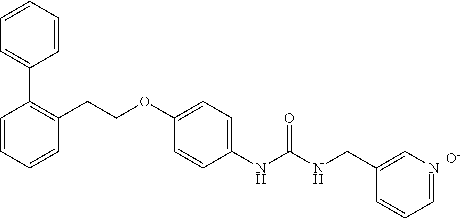 | 1-{4-[2-(Biphenyl- 2- yl)ethoxy]phenyl}- 3-[(1-oxidopyridin- 3-yl)methyl]urea | LC-MS [M + H]+ 440.1992 | |
| 821 | 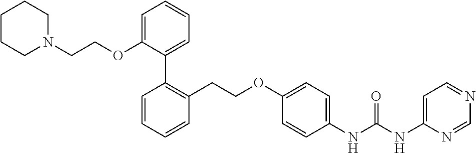 [SMILES: O=C(Nc1ccc(OCCc2ccccc2-c2ccccc2OCCN2CCCCC2)cc1)Nc1ccncn1] | 1-[4-(2-{2′-[2- (Piperidin-1- yl)ethoxy]biphenyl- 2-yl}ethoxy)phenyl]- 3-pyrimidin-4- ylurea | LC-MS [M + H]+ 538.2791 | |
| 822 | 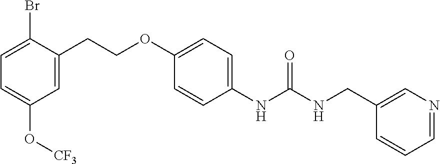 [SMILES: O=C(NCc1cccnc1)Nc1ccc(OCCc2cc(OC(F)(F)F)ccc2Br)cc1] | 1-(4-{2-[2-Bromo- 5- (trifluoromethoxy) phenyl]ethoxy} phenyl)-3-(pyridin- 3-ylmethyl)urea | LC-MS [M + H]+ 510.0633 | |
| 823 | 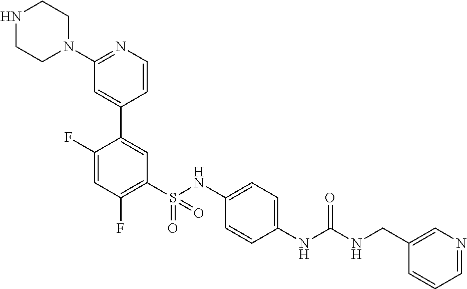 [SMILES: O=C(NCc1cccnc1)Nc1ccc(NS(=O)(=O)c2cc(-c3ccnc(N4CCNCC4)c3)c(F)cc2F)cc1] | 2,4-Difluoro-5-[2- (piperazin-1- yl)pyridin-4-yl]-N- (4-{[(pyridin-3- ylmethyl)carbamoyl] amino}phenyl) benzenesulfonamide | LC-MS [M + H]+ 580.1922 | |
| 824 | 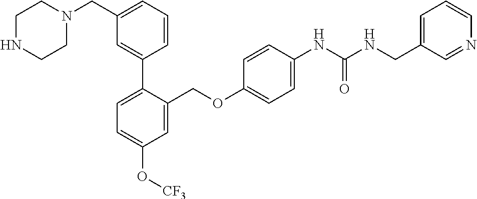 [SMILES: O=C(NCc1cccnc1)Nc1ccc(OCc2cc(OC(F)(F)F)ccc2-c2cccc(CN3CCNCC3)c2)cc1] | 1-(4-{[3′- (Piperazin-1- ylmethyl)-4- (trifluoromethoxy) biphenyl-2- yl]methoxy}phenyl)- 3-(pyridin-3- ylmethyl)urea | LC-MS [M + H]+ 592.2658 | 8.69-8.57 (m, 5H), 7.93 (d, 1H), 7.76 (d, 1H), 7.73-7.42 (m, 7H), 7.34 (s, 1H), 7.26 (d, 2H), 6.78) d, 2H), 6.75 (t, 1H), 4.91 (s, 2H), 4.36 (d, 2H), 4.10 (q, 2H), 3.31-3.10 (m, 8H) |
| 825 | 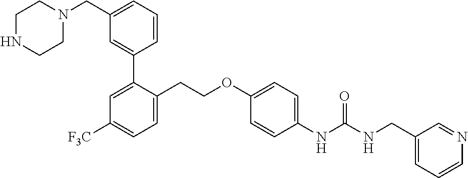 [SMILES: O=C(NCc1cccnc1)Nc1ccc(OCCc2ccc(C(F)(F)F)cc2-c2cccc(CN3CCNCC3)c2)cc1] | 1-(4-{2-[3′- (Piperazin-1- ylmethyl)-5- (trifluoromethyl) biphenyl-2- yl]ethoxy}phenyl)- 3-(pyridin-3- ylmethyl)urea | LC-MS [M + H]+ 590.2848 | 8.69-8.48 (m, 5H), 7.94-7.84 (m, 1H), 7.73 (s, 2H), 7.61- 7.37 (m, 6H), 7.23 (d, 2H), 6.71-6.64 (m, 3H), 4.33 (d, 2H), 4.10 (q, 2H), 4.02 (t, 2H), 3.20-3.08 (m, 8H), 3.04 (t, 2H) |
| 826 | 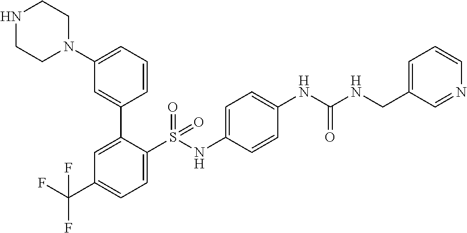 [SMILES: O=C(NCc1cccnc1)Nc1ccc(NS(=O)(=O)c2ccc(C(F)(F)F)cc2-c2cccc(N3CCNCC3)c2)cc1] | 3′-(Piperazin-1-yl)- N-(4-{[(pyridin-3- ylmethyl)carbamoyl] amino}phenyl)-5- (trifluoromethyl) biphenyl-2- sulfonamide | LC-MS [M + H]+ 611.2045 | |
| 827 | 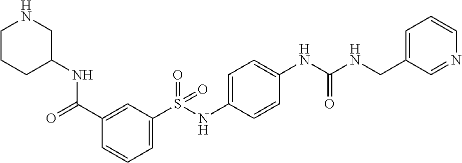 [SMILES: O=C(NCc1cccnc1)Nc1ccc(NS(=O)(=O)c2cccc(C(=O)NC3CCCNC3)c2)cc1] | N-(Piperidin-3-yl)- 3-[(4-{[(pyridin-3- ylmethyl)carbamoyl] amino}phenyl)sul- fonamoyl]benzamide | LC-MS [M + H]+ 509.1967 | |
| 828 | 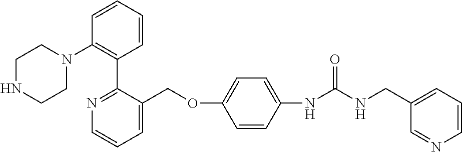 [SMILES: O=C(NCc1cccnc1)Nc1ccc(OCc2cccnc2-c2ccccc2N2CCNCC2)cc1] | 1-[4-({2-[2- (Piperazin-1- yl)phenyl]pyridin- 3-yl}methoxy) phenyl]-3-(pyridin- 3-ylmethyl)urea | LC-MS [M + H]+ 495.2488 | |
| 829 | 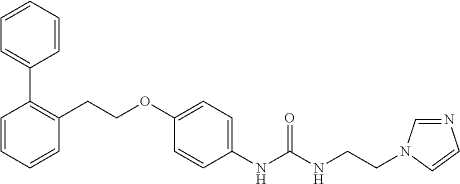 [SMILES: O=C(NCCn1ccnc1)Nc1ccc(OCCc2ccccc2-c2ccccc2)cc1] | 1-{4-[2-(Biphenyl- 2- yl)ethoxy]phenyl}- 3-[2-(1H-imidazol- 1-yl)ethyl]urea | LC-MS [M + H]+ 427.2109 | |
| 830 | 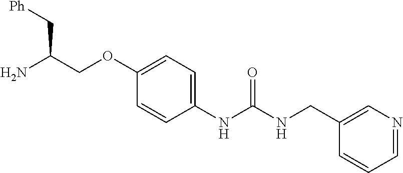 [SMILES: N[C@H](COc1ccc(NC(=O)NCc2cccnc2)cc1)Cc1ccccc1] | 1-(4-{[(2S)-2- Amino-3-phenyl- propyl]oxo}phenyl)- 3-(pyridin-3- ylmethyl)urea | LC-MS [M + H]+ 377.1970 | |
| 831 | 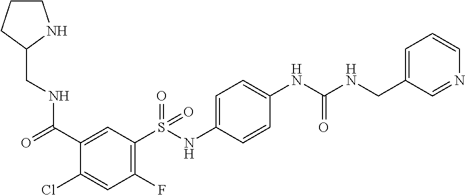 [SMILES: O=C(NCc1cccnc1)Nc1ccc(NS(=O)(=O)c2cc(C(=O)NCC3CCCN3)c(Cl)cc2F)cc1] | 2-Chloro-4-fluoro- 5-[(4-{[(pyridin-3- ylmethyl)carbamoyl] amino}phenyl) sulfamoyl]-N- (pyrrolidin-2- ylmethyl)benzamide | LC-MS [M + H]+ 561.1502 | |
| 832 | 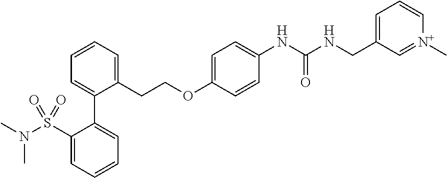 | 3-({[(4-{2-[2′- (Dimethylsulfamoyl) biphenyl-2-yl] ethoxy}phenyl) carbamoyl]amino} methyl)-1- methylpyridinium | LC-MS [M + H]+ 546.2281 | |
| 833 | 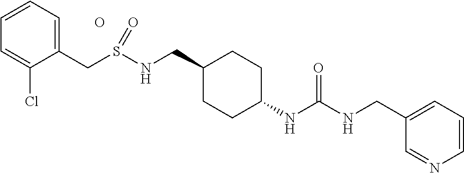 | 1-(2- Chlorophenyl)-N- [(trans-4- {[(pyridin-3- ylmethyl)carbamoyl] amino}cyclohexyl) methyl]methane sulfonamide | LC-MS [M + H]+ 451.1619 | |
| 834 | 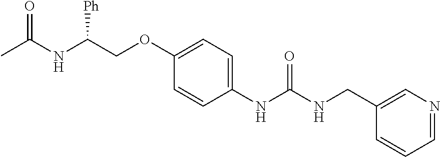 [SMILES: CC(=O)N[C@@H](COc1ccc(NC(=O)NCc2cccnc2)cc1)c1ccccc1] | N-[(1R)-1-Phenyl- 2-(4-{[(pyridin-3- ylmethyl)carbamoyl] amino}phenoxy) ethyl]acetamide | LC-MS [M + H]+ 405.1927 | |

### Biochemical and Biological Examples

### Cytotoxicity Assay

HCT116 cells were seeded in 96 well plates (Greiner Bio-One, Monroe, N.C.) and allowed to settle overnight. Test compound dissolved in dimethyl sulfoxide (DMSO) was added and drug incubation proceeded for 72 hours. When applicable, a 1000× solution of nicotinic acid (NA; Sigma-Aldrich, St. Louis, Mo.) dissolved in water was generated, and 1×NA (10 M final concentration) was added at the same time as the test compound. After 72 hour, 50 L of CellTiter-Glo Luminescent Cell Viability Assay reagent (Promega Corporation, Madison, Wis.) was added to cells in 200 L of cellular media. After a proscribed incubation period, luminescence was measured using a TopCount NXT plate reader (PerkinElmer, Waltham, Mass.).

The example compounds listed in Tables 1, 3, and 5 exhibited HCT116 cell cytotoxicity with an IC₅₀ of less than 100 nM. For example, example compound number 152 exhibited an IC₅₀ of about 55 nM, example compound number 164 exhibited an IC₅₀ of about 74 nM, example compound number 210 exhibited an IC₅₀ of about 39 nM, example compound number 605 exhibited an IC₅₀ of about 1.1 nM, example compound number 759 exhibited an IC₅₀ of about 1.9 nM, and example compound number 762 exhibited an IC₅₀ of about 2.2 nM.

Some of the example compounds listed in Tables 2, 4, and 6 exhibited an HCT116 cell cytotoxicity with an IC₅₀ of 100 nM or greater or were not tested in the cytotoxicity assay. For example, example compound number 363 exhibited an IC₅₀ of about 290 nM, example compound number 580 exhibited an IC₅₀ of about 100 nM, example compound number 613 exhibited an IC₅₀ of about 2.6 μM, example compound number 634 exhibited an IC₅₀ of about 5.0 μM, and example compound number 641 exhibited an IC₅₀ of about 3.2 μM.

### Direct Target Affinity Purification (DTAP)

Test compounds of interest were synthesized with an alkyl-amine linker to allow covalent coupling to epoxy-activated Sepharose 6B beads (GE Healthcare, Piscataway, N.J.). Sepharose beads were swollen and washed with water for 30 minutes followed by equilibration in coupling buffer (50% dimethylformamide, 50 mM Na₂CO₃). Beads were pelleted by centrifugation (15 sec at 2000×g) and the supernatant removed by aspiration. An equal volume of coupling buffer containing the linkered test compound was used to resuspend the beads. Compound concentrations in the coupling reaction ranged from 0.01 mM to 1 mM. The coupling reactions were incubated at 34° C. for 18 hrs on a rotator mixer. Ethanolamine was added to 1 M for the final 1 hour to quench the coupling reaction. Beads were washed extensively with binding buffer (1 M NaCl, 50 mM Hepes [pH 7.4], 1% Triton X-100, 1 mM EDTA and 1 mM dithiothreitol) to remove residual coupling reagents, and were then stored at 4° C.

Cellular proteins were prepared by mild sonication in lysis buffer (150 mM NaCl, 50 mM Hepes [pH 7.4], 1% Triton X-100, 1 mM EDTA and 2 mM dithiothrietol containing 1× Halt™ protease and phosphatase inhibitor cocktail [Thermo Fisher Scientific, Rockford, Ill.]). Lysates were centrifuged (20,000×g for 20 min) to remove debris, diluted to a protein concentration of ˜5 mg/mL, divided into aliquots, and stored at −80° C.

For DTAP reactions, cell lysates (˜0.5 mL per binding reaction) were thawed and the NaCl concentration adjusted to 1 M. Competitor compounds dissolved in DMSO (or a DMSO control) were then added to the lysate and incubated on ice for 5 minutes. The lysates were centrifuged at 20,000×g for 10 minutes and the cleared supernatant was transferred to a tube containing 50 μl of coupled beads. The binding reactions were incubated on a rotator mixer at 4° C. for 2 hrs, after which the beads were pelleted by centrifugation and the supernatant removed by aspiration. The beads were washed three times with 20 volumes of binding buffer, 2× with 20 volumes wash buffer (150 mM NaCl, 50 mM Hepes [pH 7.4], 1% Tween 20, 1 mM EDTA, 2 mM dithiothrietol) and finally twice with 10 volumes of 150 mM NaCl, 50 mM Hepes [pH 7.4].

During the final wash, an aliquot containing 101 of beads was transferred to a separate tube and resuspended with 15 μl of 2×SDS/PAGE loading buffer (Invitrogen Corporation, Carlsbad, Calif.) for 5 minutes at 90° C. The eluted proteins were resolved by electrophoresis on a NuPage 4-12% Bis-Tris Gel (Invitrogen Corporation, Carlsbad, Calif.) and visualized by staining with Ruby Red (Invitrogen Corporation, Carlsbad, Calif.). The remaining beads (40 μl) were processed for analysis by mass spectrometry.

This assay was used to confirm the selectivity of a subset of the compounds of the present invention for targeting Nampt.

### Liquid Chromatography

### Mass Spectrometry

Bound proteins were digested by treating the beads with trypsin as follows. After the final wash, beads were resuspended in an equal volume of trypsin digest buffer (50 mM ammonium bicarbonate, (pH 8.0), 5% acetonitrile, 1 mM calcium chloride). Samples were reduced with 5 mM DTT at 65° C. for 15 minutes and alkylated with 10 mM iodoacetamide in the dark at 30° C. for 30 minutes. Sequencing grade modified trypsin (Promega Corporation, Madison, Wis.) was added and samples digested for 1.5 hours at 37° C.

For one dimensional LC-MS/MS, 5 μl aliquots (approximately 1/10 of sample) were loaded by NanoLC-AS1 autosampler (Eksigent, Dublin, Calif.) and NanoLC-2D (Eksigent, Dublin, Calif.) in 0.1% formic acid in 5% acetonitrile onto an OPTI-PAK C₁₈ trap column (Optimize Technologies, Oregon City, Oreg.). Peptides were eluted from the trap and separated on a flame-pulled 10 cm×75 μM i.d. fused-silica capillary column (Polymicro Technologies, Phoenix, Ariz.) self-packed with Synergy Hydro C₁₈ media (Phenomenex, Torrence, Calif.). The following gradient was used: 5-15% B (0.1% formic acid in acetonitrile) in 5 minutes, 15-40% B in 60 minutes, 40-60% B in 5 minutes, 80-80% B for 10 minutes, and 5-5% B for 10 minutes. Eluted peptides were ionized directly into the LTQ-Orbitrap (Thermo Fisher Scientific, Inc., Waltham, Mass.). A full scan from m/z 300-2000 was performed in the Orbitrap at a resolution of 60,000. The top five most intense ions were selected for MS2 in the LTQ (Full FT-Big 5 IT), with a normalized collision energy of 35%.

Peptides and proteins were identified by searching the raw mass spectrometry data against a combined forward and reverse human RefSeq database. The Sequest algorithm was used with the following parameters: peptide mass tolerance=10 ppm, fragment ion tolerance=1.0 kD, 2 missed cleavages allowed, differential modification of Methionine oxidation (15.994915), 3 possible modifications per peptide, and a constant cysteine modification of 57.0215. After filtering, proteins that had a protein probability greater than 10⁻³ using Bioworks 3.0 software (Thermo Fisher Scientific, Inc., Waltham, Mass.) were identified. There was a false discovery rate of less than 0.5%. Hierarcheral clustering was done using the Bigcat software package (McAfee, K. J., et al. Mol. Cell. Proteomics. 5, 1497-1513 (2006)).

### Nampt Activity Assays

5-phosphoribosyl-1-pyrophosphate (PRPP), ATP, NaM, NaMN, Triton X-100, UDP-glucose and diaphorase were purchased from Sigma-Aldrich, St. Louis, Mo. Human NAMPT, NMN adenylyltransferase (NMNAT1) and UDP-glucose dehydrogenase (UGDH) encoding DNAs were each inserted into a house-modified E. Coli expression vector such that the expressed proteins carried an N-terminal 6×His tag. The His-tagged proteins were expressed in the BL21-AI E. Coli expression strain (Invitrogen Corporation, Carlsbad, Calif.) following induction by 0.2% L-arabinose and 0.5 mM IPTG at 30° C. Proteins were purified on Ni-NTA resin (Qiagen, Germantown, Md.).

The assay for Nampt catalytic activity was constructed based on a previously published coupled enzyme fluorometric technique, which employs NADH as ultimate analyte (Revollo, J. R. et al. Biol. Chem. 279, 50754-50763 (2004)). A substantial improvement in assay sensitivity was achieved by switching from direct detection to a resazurin/diaphorase-based fluorometric detection system for NADH (Guilbault, G. G., and Kramer, D. N. Anal. Chem. 37, 1219-1221 (1965)). The standard inhibition analyses were performed in a real-time mode in 96-well microtiter plates using 50 mM Tris-HCl, pH 7.5, 1% DMSO (v/v), 0.01% Triton X-100 (v/v), 10 mM MgCl₂, 2 mM ATP, 3 M NAM, 8 M PRPP, 50 μM Nampt, as well as the following detection reagents: 5 nM Nmnat, 200 nM Ugdh, 200 M UDP-glucose, 0.02 U/mL diaphorase and 0.25 M resazurin. Incubation of samples at room temperature for up to 3 hours was followed by quantification of fluorescence intensities at excitation and emission wavelengths of 510 nm and 590 nm, respectively, using Gemini XS plate reader (Molecular Devices, Sunnyvale, Calif.). The counter-assay intended to disqualify false positives, such as inhibitors of detection enzymes or fluorescence quenchers, was carried out essentially as described above with an exception that 1 μM NaMN was substituted for Nampt. A preparation of catalytically inactive Nampt-D313A mutant enzyme was used as a negative control for assay development.

All of the compounds of Tables 1A and 1B, 2, 3A and 3B, 4, 5, and 6 were tested using this assay. For example, example compound number 152 exhibited an vitro IC₅₀ of about 2.0 nM, example compound number 164 exhibited an vitro IC₅₀ of about 1.8 nM, example compound number 210 exhibited an vitro IC₅₀ of about 6.3 nM, example compound number 363 exhibited an vitro IC₅₀ of about 3.4 nM, example compound number 580 exhibited an vitro IC₅₀ of about 0.8 nM, example compound number 605 exhibited an vitro IC₅₀ of about 2.4 nM, example compound number 613 exhibited an vitro IC₅₀ of about 11 nM, example compound number 634 exhibited an vitro IC₅₀ of about 520 nM, example compound number 641 exhibited an vitro IC₅₀ of about 1.3 μM, example compound number 831 exhibited an vitro IC₅₀ of about 10 nM, and example compound number 797 exhibited an vitro IC₅₀ of about 10 nM.

### Assay to Measure NAD⁺ in Cellular Lysates

NAD⁺ in cells was measured by modification of existing protocols (Lee, H. I., et al. Exp. Mol. Med. 40, 246-253 (2008)). MCF-10A cells stably transduced with the PIK3CA(H1047R) oncogene were seeded in 96 well plates at very high density (100% confluence) and allowed to settle overnight. Test compound dissolved in DMSO was added and drug incubation proceeded for 20-24 hours. Cells were washed with PBS and harvested by incubation in 25 L 0.5 M perchloric acid (HClO₄) followed by vigorous shaking at 4° C. for 15 minutes. Acidic cell lysates were neutralized by adding 8 μL of 2 M KOH/0.2 M K₂HPO₄. The entire lysate volume was transferred to a centrifuge plate and spun at 3000 rpm in a table top centrifuge (4° C.) for 5 minutes to clear the precipitate. Lysate was assayed for both NAD⁺ and ATP. For NAD⁺ measurement, 10 L lysate from the centrifuged plate was added to 90 L of reaction solution in Costar 96 half-well plates (Corning, Corning, N.Y.). The final concentration of the reaction mixture was 120 M Tris-HCl, pH 7.5, 0.01% Triton X-100, 35 M UDP-Glucose, 50 nM UGDH, 0.5 M resazurin, and 0.1 unit/mL Diaphorase. Reactions were allowed to proceed for 1 hour at room temperature, after which time fluorescence was read on a Gemini plate reader as described above. For ATP measurement, 5 μL of cleared lysate was added to 195 μL PBS. 50 μL CellTiter-Glo reagent (Promega Corporation, Madison, Wis.) was added and ATP measured as described in the cytotoxicity assay methods.

### PAR Assay

To measure Poly (ADP-Ribose) Polymerase (PARP) activity, an imaging-based cellular assay was developed. MCF-10A cells stably transduced with the PIK3CA(H1047R) oncogene were seeded in 96 well plates and allowed to settle overnight. Test compound dissolved in DMSO was added and drug incubation proceeded for 20-24 hours. Under these conditions, Nampt inhibitors showed no evidence of toxicity. The next morning, hydrogen peroxide was added to the cells to a final concentration of 500 μM. After 8 minutes of hydrogen peroxide treatment, cells were fixed in 100%, −20° C. methanol. After re-hydrating and washing with PBS, cells were incubated in blocking buffer (HBSS, 1% BSA, 0.1% Tween20), and were then stained overnight with an anti-PAR mouse monoclonal antibody (Trevigen, Gaithersburg, Md.; 1:2000 dilution in blocking buffer). Cells were washed with PBS and incubated with 1:1000 of anti-mouse-Alexa488 (Invitrogen Corporation, Carlsbad, Calif.), 5 μg/mL Hoechst 33342 (Invitrogen), and 0.1 μg/mL HCS CellMask deep red (Invitrogen). Cells were washed with PBS and then stored in blocking buffer).

Images were acquired on a Pathway 855 instrument (BD Biosciences, San Jose, Calif.) using a 10× objective. Using Attovision software (BD Biosciences, San Jose, Calif.), the Hoechst signal was used to segment nuclei and the PAR signal for each nuclei in a well was subsequently averaged to generate a single value. After background subtraction using samples that were not incubated with the anti-PAR primary antibody, PAR intensity per well was graphed (Prism; GraphPad Software, Inc.; La Jolla, Calif.).

### NA Rescue and Naprt1 Expression Assays

Cell lines were treated with a fixed dose of Exemplary Compound A and screened for NA rescue and Naprt1 expression by immunoblotting and quantitative RT-PCR (Table 7). Of 176 cell lines tested, 47 did not rescue, 16 partially rescued and 113 completely rescued. The 176 cell lines included 5 normal (non-cancerous) cells and 3 primary cells (italicized in the table), all of which rescued. Naprt1 was quantified by western blotting and q-RT-PCR in 164 and 123 of the 176 cell lines, respectively. Naprt1 levels were low or undetectable in cell lines that did not rescue. A statistically significant (p value <0.0001) correlation existed between NA rescue phenotype and Naprt1 protein or mRNA expression levels.

For quantification by western blot, human tumor cell proteins were prepared from frozen cell pellets. Cell pellets were thawed and lysed in 0.5% Triton X-100, 50 mM HEPES [pH 7.4], 150 mM NaCl, 1 mM EDTA, 10% glycerol, and 1 mM DTT for 30 minutes at 4° C. After centrifugation to remove cellular debris, protein concentration was determined using the BCA (Sigma BCA1-1KT) or CBQCA protein assay kits (Molecular Probes #C-6667). Ruby Red staining of SDS-PAGE gels was used to confirm protein loading.

For immunoblot detection, equivalent protein amounts were resolved by electrophoresis and transferred to nitrocellulose membrane. Membranes were blocked in Starting Block T20 (TBS) (Thermo Scientific #37543) and were probed with anti-Naprt (Proteintech Group 13549-1-AP or anti-Gapdh (Calbiochem #CB1001) antibodies. HRP-conjugated secondary antibodies (Santa Cruz Biotechnology) and Super Signal West Dura Extended Duration Substrate (Thermo Scientific #34075) were used for detection. Protein signals were quantified by imaging using an EC3 imaging system (UVP Bioimaging Systems) and VisionWorksSL software. The dynamic range of signal detection was enhanced by utilizing multiple exposure times. Naprt protein levels were calculated as a percentage of the cognate signal detected in the HCT 116 cell lysate.

For quantification by qRT-PCR, Untreated cell pellets were collected lysed in RLT buffer with 1% β-Mercaptoethanol. RNA was isolated using an RNeasy spin column kit (Qiagen 74104), loaded in triplicate to a 96-well plate at 11 ng total RNA/well, and probed for NAPRT1 with the TaqMan primer set Hs00292993_ml, using the QuantiTect probe RT-PCR kit (Qiagen 204443), with a final sample volume of 25 ul/well. Relative NAPRT expression was assayed on the Applied Biosystems 7300 Real-Time PCR system thermal cycler. The plate was heated to 50° C. for 30 minutes, followed by 95° C. for 15 minutes, followed by 40 cycles alternating between 95° C. for 15 seconds and 60° C. for 1 minute. Data was collected during the 60° C. step of each cycle, and cycle threshold values were interpolated onto a dilution curve of total RNA from the cell line SK-BR-3 to give relative values of the initial NAPRT mRNA concentration for each sample. The average RNA concentration for each cell line was then presented relative to the expression seen in the cell line SK− BR-3 as a percentage.

TABLE 7

| Cell line | Tissue | NA rescue | Cell line | Tissue | NA rescue |
|---|---|---|---|---|---|
| T24 | bladder | yes | NCI-H1993 | lung (NSCLC) | yes |
| KINGS-1 | brain; anaplastic | no | NCI-H2030 | lung (NSCLC) | yes |
| | astrocytoma | | | | |
| | (glioma) | | | | |
| CCF-STTG1 | brain; astrocytoma | yes | NCI-H2110 | lung (NSCLC) | yes |
| SNB-75 | brain; astrocytoma | yes | NCI-H2228 | lung (NSCLC) | yes |
| SW1088 | brain; astrocytoma | yes | NCI-H226 | lung (NSCLC) | yes |
| SW1783 | brain; astrocytoma | yes | NCI-H23 | lung (NSCLC) | yes |
| SF-268 | brain; astrocytoma, | no | NCI-H441 | lung (NSCLC) | yes |
| | anaplastic | | | | |
| SNB-19 | brain; glioblastoma | no | NCI-H596 | lung (NSCLC) | partial |
| U251 | brain; glioblastoma | no | NCI-H69 | lung (small cell) | no |
| DBTRG-05MG | brain; glioblastoma | yes | NCI-H146 | lung (small cell) | yes |
| KNS-42 | brain; glioma | no | NCI-H209 | lung (small cell) | yes |
| Hs683 | brain; glioma | yes | NCI-H345 | lung (small cell) | yes |
| no. 10 | brain; glioma, | no | SHP-77 | lung (small cell) | yes |
| | anaplastic | | | | |
| no. 11 | brain; glioma, | partial | KARPAS-299 | lymphocytic | no |
| | anaplastic | | | leukemia | |
| SF-539 | brain; gliosarcoma | yes | CCRF-CEM | lymphocytic | yes |
| | | | | leukemia | |
| M059J | brain; malignant | no | Jurkat | lymphocytic | yes |
| | glioblastoma; | | | leukemia | |
| | glioma | | | | |
| PFSK-1 | brain; malignant | partial | MOLT4 | lymphocytic | yes |
| | neuroectodermal | | | leukemia | |
| | tumor | | | | |
| Daoy | brain; | yes | THP-1 | lymphocytic | yes |
| | medulloblastoma | | | leukemia | |
| CHLA-90 | brain; | no | MONO- | lymphocytic | partial |
| | neuroblastoma | | MAC-6 | leukemia | |
| IMR-32 | brain; | no | Daudi | lymphoma | no |
| | neuroblastoma | | | | |
| LA-N-6 | brain; | yes | H9 | lymphoma | no |
| | neuroblastoma | | | | |
| SMS-KCNR | brain; | yes | NAMALWA | lymphoma | no |
| | neuroblastoma, | | | | |
| | ALK (R1275Q). | | | | |
| SK-N-SH | brain; | no | SR-786 | lymphoma | no |
| | neuroblastoma, | | | | |
| | metastatic | | | | |
| SH-SY5Y | brain; | no | SU-DHL-1 | lymphoma | no |
| | neuroblastoma; | | | | |
| | bone marrow met. | | | | |
| SK-N-FI | brain; | no | L-82 | lymphoma | yes |
| | neuroblastoma; | | | | |
| | bone marrow met. | | | | |
| SK-N-MC | brain; | no | Ramos | lymphoma | yes |
| | neuroepithelioma; | | | | |
| | supra-orbital met. | | | | |
| H4 | brain; neuroglioma | partial | SU-DHL-10 | lymphoma | yes |
| KELLY | brain; | no | U-937 | lymphoma | yes |
| | oligodendroglioma | | | | |
| BT-474 | breast | yes | DEL | lymphoma | partial |
| DU4475 | breast | yes | SR | lymphoma | partial |
| HCC1937 | breast | yes | SU-DHL-8 | lymphoma | partial |
| MCF7 | breast | yes | SUP-M2 | lymphoma | partial |
| MDA-MB-231 | breast | yes | UACC-257 | melanoma | yes |
| MDA-MB-436 | breast | yes | MALME-3M | melanoma (lung | yes |
| | | | | metastasis) | |
| SK-BR-3 | breast | yes | A2058 | melanoma | yes |
| | | | | (lymph node | |
| | | | | metastasis) | |
| COLO320DM | colorectal | no | NIH-3T3 | mouse; | no |
| | | | | fibroblast | |
| COLO320HSR | colorectal | no | Hepa1-6 | mouse; | partial |
| | | | | hepatoma | |
| DLD-1 | colorectal | yes | RAW264.7 | mouse; | no |
| | | | | leukemia | |
| HCC2998 | colorectal | yes | MLE-12 | mouse; lung | no |
| HCT-15 | colorectal | yes | KU812 | myelogenous | no |
| | | | | leukemia | |
| HCT-8 | colorectal | yes | HL-60 | myelogenous | yes |
| | | | | leukemia | |
| KM12 | colorectal | yes | K562 | myelogenous | yes |
| | | | | leukemia | |
| LS174T | colorectal | yes | MOLM-13 | myelogenous | yes |
| | | | | leukemia | |
| RKO | colorectal | yes | MV-4-11 | myelogenous | yes |
| | | | | leukemia | |
| SK-CO-1 | colorectal | yes | NB-4 | myelogenous | yes |
| | | | | leukemia | |
| SNU-C2B | colorectal | yes | NOMO-1 | myelogenous | partial |
| | | | | leukemia | |
| SW-48 | colorectal | yes | SKM-1 | myelogenous | partial |
| | | | | leukemia | |
| SW480 | colorectal | yes | K562 | myelogenous | yes |
| | | | | leukemia | |
| | | | | (CML) | |
| SW620 | colorectal | yes | MEG-01 | myelogenous | yes |
| | | | | leukemia | |
| | | | | (CML) | |
| Hs414.T | fibrosarcoma | yes | AMO-1 | myeloma | no |
| Hs93.T | fibrosarcoma | yes | U266 | myeloma | no |
| SW684 | fibrosarcoma | yes | KMS-11 | myeloma | yes |
| SW872 | fibrosarcoma | yes | MC/CAR | myeloma | yes |
| HepG2 | hepatocellular | no | MM.1S | myeloma | yes |
| | carcinoma | | | | |
| Huh7 | hepatocellular | yes | MOLP-8 | myeloma | partial |
| | carcinoma | | | | |
| SNU182 | hepatocellular | yes | RPMI-8226 | myeloma | partial |
| | carcinoma | | | | |
| SNU449 | hepatocellular | yes | JJN3 | myeloma | yes |
| | carcinoma | | | (plasma cell | |
| | | | | leukemia) | |
| ACHN | kidney | yes | HOS | osteosarcoma | no |
| BEAS-2B | lung (normal) | yes | MG-63 | osteosarcoma | no |
| IMR-90 | lung (normal) | yes | U-2 OS | osteosarcoma | no |
| MRC-5 | lung (normal) | yes | Saos-2 | osteosarcoma | yes |
| Wi-38 | lung (normal) | yes | SJSA1 | osteosarcoma | yes |
| HCC78 | lung (NSCLC) | no | SK-ES-1 | osteosarcoma | yes |
| NCI-H322 | lung (NSCLC) | no | OVCAR-3 | ovary | yes |
| A549 | lung (NSCLC) | yes | UWB1.289 | ovary | yes |
| Calu-1 | lung (NSCLC) | yes | AsPC-1 | pancreas | yes |
| Calu-6 | lung (NSCLC) | yes | BxPC-3 | pancreas | yes |
| EKVX | lung (NSCLC) | yes | Capan-1 | pancreas | yes |
| HOP18 | lung (NSCLC) | yes | CFPAC-1 | pancreas | yes |
| HOP62 | lung (NSCLC) | yes | Hs766T | pancreas | yes |
| HOP92 | lung (NSCLC) | yes | Panc-1 | pancreas | yes |
| NCI-H1299 | lung (NSCLC) | yes | PBMC | primary blood | yes |
| NCI-H1437 | lung (NSCLC) | yes | SAEC | primary lung | yes |
| NCI-H1568 | lung (NSCLC) | yes | keratinocytes | primary skin | yes |
| NCI-H1792 | lung (NSCLC) | yes | DU145 | prostate | yes |
| NCI-H1944 | lung (NSCLC) | yes | LNCAP | prostate | yes |
| | | | MALME-3 | skin (normal) | yes |

Additional cancer cell lines were treated with Exemplary Compounds A, C, D, E, F, G and H (identified below) (Table 8). The NA rescue phenotype of a particular cancer cell line was maintained for all Nampt inhibitors tested.

TABLE 8

| | | Ex. | Ex. | Ex. | Ex. | Ex. | Ex. | Ex. |
|---|---|---|---|---|---|---|---|---|
| | | Comp. | Comp. | Comp. | Comp. | Comp. | Comp. | Comp. |
| Cell line | Tissue | A | C | D | E | F | G | H |
| HCT-116 | colorectal | yes | yes | yes | yes | yes | yes | yes |
| HT-1080 | fibrosarcoma | no | no | no | no | no | no | no |
| NCI-N87 | gastric | yes | yes | yes | yes | yes | yes | yes |
| MiaPaCa2 | pancreatic | no | no | no | no | no | no | no |
| HCC827 | NSCLC | no | no | no | no | no | no | no |
| NCI-H460 | NSCLC | no | no | no | no | no | no | no |
| COLO- | colorectal | yes | yes | yes | yes | yes | yes | yes |
| 205 | | | | | | | | |
| SU-DHL- | DLBCL (NHL) | partial | partial | partial | partial | partial | partial | partial |
| 4 | | | | | | | | |
| SU-DHL- | DLBCL (NHL) | no | no | no | no | no | no | no |
| 5 | | | | | | | | |
| DB | DLBCL (NHL) | partial | partial | partial | partial | partial | partial | partial |
| OCI-Ly19 | DLBCL (NHL) | yes | yes | yes | yes | yes | yes | yes |
| OPM-2 | multiple | no | no | no | no | no | no | no |
| | myeloma | | | | | | | |
| NCI-H929 | multiple | no | no | no | no | no | no | no |
| | myeloma | | | | | | | |
| U-87MG | glioma | no | no | no | no | no | no | no |
| A172 | glioma | no | no | no | no | no | no | no |
| SF-295 | glioma | no | no | no | no | no | no | no |
| NCI- | NSCLC | no | no | no | no | no | no | no |
| H1650 | | | | | | | | |
| NCI-H522 | NSCLC | no | no | no | no | no | no | no |
| DMS-114 | SCLC | yes | yes | yes | yes | yes | yes | yes |
| NCI-H82 | SCLC | yes | yes | yes | yes | yes | yes | yes |
| OVCAR-8 | ovarian | yes | yes | yes | yes | yes | yes | yes |
| HT29 | colorectal | yes | yes | yes | yes | yes | yes | yes |

### Assays of Synergy Between Nampt Inhibitors and Various Chemotherapeutic Compounds

As noted above, Nampt inhibition has been shown to sensitize cells to the effects of various chemotherapeutic or cytotoxic agents. Specifically, Nampt inhibition has been shown to sensitize cells to amiloride, mitomycin C, N-methyl-N′-nitro-N-nitrosoguanidine (MNNG), melphalan, daunorubicin, cytarabine (Ara-C), etoposide, and the lactate dehydrogenase inhibitor FX11 (Ekelund, S. et al. Chemotherapy 48:196-204 (2002); Rongvaux, A. et al. The Journal of Immunology 181(7):4685-95 (2008); Martinsson, P. et al. British Journal of Pharmacology 137:568-73 (2002); Pogrebniak, A. et al. European Journal of Medical Research 11(8):313-21 (2006) Le, et al., Proceedings of the National Academia of Sciences 107(5):2037-2042 (2010)). Although the mechanism(s) behind this synergy between Nampt inhibitors and other cell killing agents has not been fully explored, Nampt inhibition causes a drop in cellular levels of NAD⁺ at doses and times of exposure that are not overtly toxic to the cell. In the case of HCT116 cells, it has been discovered that there is a “6% threshold,” in which cell death does not occur until NAD⁺ levels drop to approximately 6% of normal levels. Without wishing to be bound by theory, it was hypothesized that these sub-lethal NAD⁺ drops will render a cell vulnerable to other cytotoxic agents, and particularly to compounds which activate the DNA repair enzyme poly(ADP-ribose) polymerase (PARP), since PARP requires NAD⁺ as a substrate and consumes NAD⁺ during its enzymatic action (Kim, M. Y. et al. Genes & Development 19:1951-67 (2005); FIG. 1, top).

This hypothesis was tested by determining the drug interaction (synergy, additivity, or antagonism) of 19 different cytotoxic or chemotherapeutic compounds of various categories, along with a known Nampt inhibitor, as a positive control. Nineteen chemotherapeutic compounds were chosen based upon their clinical relevance and their likelihood of synergizing with Nampt inhibitors based upon the PARP model (FIG. 1). Experiments were conducted in HCT116 cells. This cell type was used extensively in the studies of the cytotoxicity of the compounds of the present invention. Further, as HCT116 cells are commonly used in xenograft cancer models, it was hypothesized that cellular experiments might provide insights into how best to conduct subsequent in vivo studies of synergy. For compound combination analyses, the MacSynergy™ II protocol and program were utilized, following the recommendations of the developers (Prichard and Shipman, 1990). Prior to combinations of compounds, dose curves of cells treated with a single compound were generated to define relevant compound doses to use in the combination analysis. Typically, relevant doses were those found in the inflection portion of a sigmoidal dose-response curve. Utilizing these optimized conditions, cells were dosed with Nampt inhibitor plus the test compound at a variety of concentrations of each, and viability was assessed using CellTiter-Glo. Data was processed using the MacSynergy™ II algorithm, which subtracted from actual data a prediction of compound additivity. Thresholds for meaningful synergy were defined based upon the recommendations of the developers (Prichard and Shipman, 1990).

Of the 19 various chemotherapeutic compounds tested, 9 displayed reproducible and quantitatively significant synergy with a known Nampt inhibitor. The compounds showing synergy included the DNA alkylating agents methyl methanesulfonate (MMS), mechlorethamine, and streptozotocin (a therapy for pancreatic cancer). Some alkylating agents can synergize with Nampt inhibitors due to their ability to activate PARP and depress NAD⁺ levels in cells (Miwa, M. and Masutani, M. Cancer Science 98(10):1528-35 (2007); Kim, M. Y. et al. Genes & Development 19:1951-67 (2005)). Somewhat unexpectedly, three clinically relevant drugs involved in nucleotide synthesis (i.e., 5-fluorouracil (5-FU), raltitrexed, and methotrexate) also synergized with the Nampt inhibitor. While the locus of action of each of these three drugs is different, all either directly or indirectly inhibit the enzyme thymidylate synthase (TS). TS inactivation is know to cause an imbalance in nucleotide pools that subsequently promotes aberrant uracil incorporation into DNA (Berger S. H. et al. Biochemical Pharmacology 76:697-706 (2008)). The mechanism of synergy between 5-FU and Nampt inhibitors was investigated and it was discovered that 5-FU in HCT116 cells was a PARP activator, and that activation of PARP was essential for the synergy between 5-FU and Nampt inhibitors (FIG. 1A).

The initial experiments demonstrated that 5-FU and Nampt inhibitors did not synergize in all cells tested, and in these cells lacking synergy, 5-FU did not cause detectable PARP activation. These results suggested that uracil incorporation into DNA either does not occur in all cells treated with 5-FU, or that PARP is only activated in certain cells in response to uracil incorporation into DNA. The observation of cell-specific synergy between 5-FU and Nampt inhibitors could be therapeutically useful as a mechanism of expanding therapeutic window. Of further note, it is believed the relationship uncovered between 5-FU, PARP activation, and Nampt inhibition is a new discovery.

Finally, it was observed that the proteosome inhibitor bortezomib, the PI3K/mTOR inhibitor PI-103, and the tyrosine kinase inhibitor dasatinib all synergized with the Nampt inhibitor. The synergy of these three compounds with the Nampt inhibitor was unexpected.

In HCT116 cells, the potent and selective PARP inhibitor olaparib failed to synergize with Nampt inhibitors—in fact antagonism was observed, in which olaparib protected cells somewhat from Nampt inhibitor-induced death. This was not fully unexpected, as PARP inhibitors are relatively benign to cells (like HCT116 cells) that have a functional homologous recombination (HR) system to repair double stranded DNA damage (Ashworth A. Journal of Clinical Oncology 26(22):3785-90 (2008)). In fact, the model (FIG. 1A) predicts that inhibiting an enzyme, such as PARP, that consumes NAD⁺ would protect HR-proficient cells from Nampt inhibition. However, in cells that have lost the function of BRCA tumor suppressors, HR function is compromised, and these cells are killed by PARP inhibitors (Ashworth A. (2008) Journal of Clinical Oncology 26(22):3785-90). Thus, it was hypothesized that PARP inhibitors, while being antagonistic with Nampt inhibitors in most cells, would be synergistic in cells with BRCA mutations that render the cells HR-deficient (FIG. 1B). Indeed, in MDA-MB-436 cells, which have a loss of BRCA1 function, Nampt inhibitors (a known Nampt inhibitor, Exemplary Compound A and Exemplary Compound I, both Exemplary Compounds identified hereinafter) and the PARP inhibitor olaparib synergized in causing cell death. This result is particularly encouraging as it suggests that the drug combination of one of the compounds of the present invention plus a PARP inhibitor would be antagonistic in normal cells (FIG. 1A), but synergistic in cells that have lost BRCA tumor suppressor function (FIG. 1B). Of further significance to these findings, it is becoming clear that other routes of HR deficiency in oncogenesis (other than BRCA sequence mutation) could also lead to sensitivity to PARP inhibition plus Nampt inhibitor combination therapy. These additional mutations, which lead to a “BRCAness” phenotype, include, as documented in ovarian cancers, BRCA1 promoter methylation and upregulation of BRCA inhibitors, such as the protein EMSY (Bast R. C. and Mills G. B. Journal of Clinical Oncology 28(22):3545-8 (2010)). Further studies have demonstrated that mutation of the tumor suppressor gene phosphatase and tensin homolog (PTEN), a gene frequently mutated in a variety of cancers, reduces HR function and sensitizes cells to PARP inhibitors (Mendes-Pereira A. M. et al. EMBO Molecular Medicine 1:315-322 (2009)). Providing more evidence for the BRCAness model of PARP inhibitor sensitivity, in a cell biological study using RNA interference, mutation of any of 12 different genes functionally important for HR sensitized cells to PARP inhibitors (McCabe et al. Cancer Research 66(16): 8109-15 (2006)). Finally, a recent paper has demonstrated that cells in hypoxic conditions, such as those found in the center of virtually all solid tumors, are selectively killed by PARP inhibitors (Chan et al. Cancer Research 70(2): 8045-54 (2010)). Thus, there are many clinical opportunities for PARP inhibitors and Nampt inhibitors to be combined to treat a wide variety of cancers.

These studies were expanded to investigate synergistic combinations of Nampt inhibitors and standards of care in particular cancer types. Cancer cell lines used in these studies represented cancer types found to be sensitive to Nampt inhibition [e.g. non-Hodgkins lymphoma, multiple myeloma, glioma, non-small cell lung carcinoma (NSCLC), small cell lung carcinoma (SCLC), ovarian cancer and colorectal cancer]. Standards of care in these cancer types tested in synergy experiments included: 4-HC (the pre-activated form of cyclophosphamide), doxorubicin, vincristine, prednisolone, dexamethasone, melphalan, thalidomide, bortezomib, temozolomide, cisplatin, paclitaxel, gefitinib, 5-FU, oxaliplatin, irinotecan, and etoposide. Synergistic cytotoxicity was found when Nampt inhibitors (Exemplary Compound A and Exemplary Compound C, both identified hereinafter) were combined with 4HC in small-cell lung cancer (SCLC) and glioma, temozolomide in glioma, and 5-FU in colon cancer.

### Nampt Inhibition Proves Cytotoxic to a Wide Variety of Cancer Cell Types

Nampt is most active in adipose tissue, liver, kidney, immune cells, and intestine (Bogan, K. L and Brenner, C. Nicotinic acid, nicotinamide, and nicotinamide riboside: a molecular evaluation of NAD⁺ precursor vitamins in human nutrition. Annu Rev Nutr. 28:115-305 (2008); and Revollo J R, et al. Nampt/PBEF/Visfatin regulates insulin secretion in beta cells as a systemic NAD biosynthetic enzyme. Cell Metab. November; 6(5):363-75 (2007)). Nevertheless, we sought to find out whether cancer cell lines of other origins are sensitive to Nampt inhibition.

Exponentially growing cells were plated in fresh growth media in a 96-well black, flat, clear-bottomed polystyrene microtiter plate (Packard View Plate 6005182). Twenty-four hours later, compounds were added from serial dilutions prepared in DMSO from 50 mM DMSO stock solutions. Each concentration of inhibitor was tested in duplicate at a final DMSO concentration of 0.4%. After 72 or 96 hours incubation, cell viability was quantified by measuring intracellular ATP levels using CellTiter-Glo (Promega). Luminescence data was collected on a TopCount NXT plate reader (PerkinElmer). Experimental values were normalized to solvent controls and plotted versus compound concentration to determine the concentration required for a 50% reduction in cell viability.

Using the Cytotoxicity Assay outlined above, several exemplary compounds of the present invention (“Exemplary Compounds A, B, C, D, E, F, G, and H), and a known Nampt inhibitor (“Control Nampt Inhibitor”) were tested and the results are shown in Tables 9A and 9B. Exemplary Compound A is a compound represented by Formula IIIb7. Exemplary Compounds B and I are compounds represented by Formula IIIb5. Exemplary Compounds C, D, and H are compounds represented by Formula IIIb9. Exemplary Compound E, F, and G are compounds represented by Formula IIIb8. Killing was nearly complete (>80%) with all three compounds after 3 days, and was complete in all lines after 7 days. These data demonstrate that a wide variety of cancer cell types are susceptible to killing by the compounds of the present invention. Units are TC₅₀ (“Toxic Concentration required to cause 50% growth inhibition”) in nanoMolar (nM).

TABLE 9A

| | | Exemplary | | Exemplary | | Exemplary | Control Nampt | |
|---|---|---|---|---|---|---|---|---|
| | Cancer | Compound A | | Compound B | | Compound C | Inhibitor | |
| Cell Line | type | 3 DAY | 7 DAY | 3 DAY | 7 DAY | 3 DAY | 3 DAY | 7 DAY |
| COLO205 | colon | 0.5 | 0.3 | 1.5 | 1.3 | 0.53 | 2.0 | 1.1 |
| DU145 | prostate | 3.9 | 2.7 | 16.5 | 8.9 | | 9.7 | 5.7 |
| DU4475 | breast | 0.1 | 0.1 | 0.4 | 0.1 | | 0.8 | 0.2 |
| HCC827 | NSCLC | 8.0 | 1.5 | 17.7 | 4.6 | 7.5 | 30.6 | 6.5 |
| HCT116 | colon | 0.6 | 0.4 | 2.4 | 2.3 | 0.51 | 3.5 | 1.6 |
| HCT-15 | colon | 0.7 | 1.0 | 13.7 | | | 3.8 | 3.2 |
| HOP92 | NSCLC | 17.0 | 4.3 | 44.6 | 10.1 | | 39.6 | 9.0 |
| HT1080 | sarcoma | 1.0 | 0.6 | 3.4 | 2.2 | 0.96 | 4.6 | 2.1 |
| HT29 | colon | 1.4 | 1.1 | 4.7 | 4.5 | 4.9 | 7.1 | 2.8 |
| KM12 | colon | 0.9 | 0.4 | 3.5 | 1.4 | | 4.4 | 1.8 |
| MDA-MB-231 | breast | 10.0 | 7.5 | 37.3 | 26.0 | | 31.0 | 17.4 |
| MIA PaCa-2 | pancreatic | 1.8 | 0.4 | 4.9 | 4.1 | 3.8 | 7.9 | 1.8 |
| NCI-H460 | NSCLC | 15.4 | | 53.2 | 63.5 | 15 | 36.9 | 19.8 |
| NCI-H522 | NSCLC | 1.0 | 0.4 | 2.8 | 1.3 | 0.97 | 4.0 | 1.2 |
| NCI-H69 | SCLC | 1.0 | | 3.0 | | | 3.3 | |
| NCI-N87 | gastric | 0.3 | 0.2 | 1.1 | 0.3 | 0.21 | 2.5 | 0.9 |
| OPM-2 | myeloma | 1.5 | | 3.8 | | 1.5 | 5.7 | |
| OVCAR3 | ovarian | 1.1 | 0.4 | 2.5 | 0.9 | | 3.7 | 1.3 |
| SU-DHL-4 | lymphoma | 1.5 | | | | 0.23 | | |
| SU-DHL-5 | lymphoma | 0.9 | | | | 0.19 | | |
| DB | lymphoma | 3.5 | | | | 1.1 | | |
| OCI-Ly19 | lymphoma | 1.2 | | | | 0.38 | | |
| NCI-H929 | myeloma | 2.5 | | | | 1.4 | | |
| U-87MG | glioma | 23 | | | | 17 | | |
| A172 | glioma | 1.1 | | | | 0.12 | | |
| SF-295 | glioma | 1.5 | | | | 0.37 | | |
| NCI-H1650 | NSCLC | 2.5 | | | | 0.28 | | |
| DMS-114 | SCLC | 0.16 | | | | 0.46 | | |
| NCI-H82 | SCLC | 1.1 | | | | 0.23 | | |

TABLE 9B

| | | Exemplary | Exemplary | Exemplary | Exemplary | Exemplary |
|---|---|---|---|---|---|---|
| | Cancer | Compound | Compound | Compound | Compound | Compound |
| Cell Line | type | D | E | F | G | H |
| COLO205 | colon | 1.5 | 0.22 | 1.1 | 0.14 | 8.6 |
| DU145 | prostate | | | | | |
| DU4475 | breast | | | | | |
| HCC827 | NSCLC | 62 | 14 | 19 | 11 | 325 |
| HCT116 | colon | 5.5 | 0.46 | 3.5 | 0.8 | 15 |
| HCT-15 | colon | | | | | |
| HOP92 | NSCLC | | | | | |
| HT1080 | sarcoma | 15 | 1.4 | 4.9 | 0.97 | 38 |
| HT29 | colon | 10 | 3.1 | 6.5 | 1.4 | 62 |
| KM12 | colon | | | | | |
| MDA-MB-231 | breast | | | | | |
| MIA PaCa-2 | pancreatic | 17 | 4.3 | 16 | 2.3 | 68 |
| NCI-H460 | NSCLC | 211 | 65 | 69 | 39 | 795 |
| NCI-H522 | NSCLC | 2.8 | 0.39 | 1.1 | 0.14 | 14 |
| NCI-H69 | SCLC | | | | | |
| NCI-N87 | gastric | 1.8 | 0.24 | 0.8 | 0.18 | 7.3 |
| OPM-2 | myeloma | 4.2 | 1.8 | 2.3 | 0.6 | 35 |
| OVCAR3 | ovarian | | | | | |
| SU-DHL-4 | lymphoma | 2.5 | 0.28 | 1.8 | 0.11 | 7.4 |
| SU-DHL-5 | lymphoma | 3.1 | 0.08 | 0.35 | 0.08 | 1.1 |
| DB | lymphoma | 5 | 1.2 | 4.3 | 0.66 | 19 |
| OCI-Ly19 | lymphoma | 0.5 | 0.22 | 0.67 | 0.08 | 4.8 |
| NCI-H929 | myeloma | 5.2 | 1.5 | 3.9 | 0.23 | 17 |
| U-87MG | glioma | 62 | 74 | 43 | 17 | 1600 |
| A172 | glioma | 1.8 | 0.36 | 1.1 | 0.22 | 6.7 |
| SF-295 | glioma | 41 | 0.91 | 15 | 14 | 15 |
| NCI-H1650 | NSCLC | 4.3 | 0.59 | 1.7 | 0.25 | 9.6 |
| DMS-114 | SCLC | 15 | 0.82 | 3.5 | 3.3 | 4.5 |
| NCI-H82 | SCLC | 0.73 | 0.12 | 0.26 | 0.08 | 1.8 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be understood that certain changes and modifications can be practiced within the scope of the appended claims.

1. A compound having a structure according to Formula I 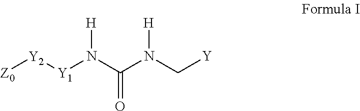
and pharmaceutically acceptable salts and solvates thereof;
wherein:
Y is phenyl, 2-pyridinyl, 3-pyridinyl, or 4-pyridinyl, wherein any ring carbon is optionally independently substituted with halo, C₁₋₅alkyl, nitro, cyano, C₁₋₅alkoxy, C-amido, N-amido, C-carboxy, O-carboxy, sulfonamide, amino, hydroxyl, mercapto, alkylthio, sulfonyl, or sulfinyl;
Y₁ is divalent carbocycle, divalent heterocycle, divalent phenyl or divalent heteroaryl, wherein any ring atom is optionally independently substituted with halo, C₁₋₅alkyl, nitro, cyano, trihalomethyl, C₁₋₅ alkoxy, C-amido, N-amido, sulfonamide, amino, aminosulfonyl, hydroxyl, mercapto, alkylthio, sulfonyl, or sulfinyl, or
Y₁ is C₂₋₈alkylene or C₂₋₈alkenylene, optionally interrupted one, two, or three times by —O—, —S—, —S(═O)—, —S(═O)₂—, —OC(═O)N(R)—, —N(R)C(═O)O—, —C(═O)N(R)—, —N(R)C(═O)—, —N(R)C(═O)N(R)—, —N(R)—, —C(═O)—, —OC(═O)—, —C(═O)O—, —OS(═O)₂N(R)—, —N(R)S(═O)₂O—, —SC(═O)—, —C(═O)S—, —OC(═S)N(R)—, —N(R)C(═S)O—, —C(═S)N(R)—, —N(R)C(═S)—, —N(R)C(═S)N(R)—, —C(═S)—, —OC(═S)—, —C(═S)O—, —S(═O)₂N(R)—, —N(R)S(═O)₂—, —S(═O)₂N(R)C(═O)—, or —C(═O)N(R)S(═O)₂—;
Y₂ is —OCH₂—, —SCH₂—, —N(R)CH₂—, —N(R)C(═O)—, —C(═O)N(R)—, —S(═O)₂CH₂—, —S(═O)CH₂—, —CH₂O—, —CH₂CH₂O—, —CH₂S—, —CH₂N(R)—, —CH₂S(═O)₂—, —CH₂S(═O)—, —C(═O)O—, —C(═O)—, —SO₂N(R)—, —N(R)SO₂—, ethylene, propylene, n-butylene, —O—C₁₋₄ alkylene-N(R)C(═O)-, —O—C₁₋₄alkylene-C(═O)N(R)—, —N(R)C(═O)—C₁₋₄ alkylene-O—, —C(═O)N(R)—C₁₋₄ alkylene-O—, —C₁₋₄alkylene-S(═O)₂—, —C₁₋₄alkylene-S(═O)—, —S(═O)₂—C₁₋₄ alkylene-, —S(═O)—C₁₋₄ alkylene-, —C₁₋₄ alkylene-SO₂N(R)—, —C₁₋₄ alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄alkylene-O—, —S—C₁₋₄ alkylene-, —C₁₋₄alkylene-S—, —C₁₋₄alkylene-S—C₁₋₄ alkylene-, —N(R)—C₁₋₄ alkylene-, —C₁₋₄ alkylene-N(R)—, —C₁₋₄alkylene-N(R)—C₁₋₄ alkylene-, —C₁₋₄ alkylene-C(═O)—O—C₁₋₄ alkylene-, —C₁₋₄alkylene-O—C(═O)—C₁₋₄alkylene-, —C₁₋₄alkylene-C(═O)—N(R)—C₁₋₄ alkylene-, —C₁₋₄ alkylene-N(R)—C(═O)—C₁₋₄ alkylene-, —C(═O)—N(R)—C₁₋₄ alkylene-SO₂N(R)—, or —N(R)—C(═O)—C₁₋₄ alkylene-SO₂N(R)—;
Z₀ is carbocycle, cycloalkyl, cycloalkenyl, heterocycle, heterocyclonoyl, aryl, heteroaryl, carbocycloalkyl, heterocyclylalkyl, arylalkyl, arylalkenyl, heteroarylalkyl, heteroarylalkenyl, heteroarylalkynyl, or arylalkynyl, wherein any of the foregoing groups are optionally substituted at least once with alkyl, alkylene, alkenyl, alkenylene, alkynyl, alkynylene, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide;
wherein any alkylene or alkenylene group is optionally independently substituted with C₁₋₄ alkyl, halo, C₁₋₄haloalkyl, or C₃ or C₄ cycloalkyl;
wherein for the purpose of Y₁, R is H, halo, C₁₋₄alkyl, C₁₋₄alkenyl, or C₁₋₄alkynyl;
wherein for the purpose of Y₂, R is H, halo, C₁₋₅alkyl, C₁₋₅alkenyl, C₁₋₅alkynyl, or is methylene or ethylene that forms a 5- or 6-membered heterocycle with a carbon atom of Z₀; and
with the proviso that the compound is NOT:
ethyl 3-(pyridin-3-yl)-4-({4-[(3-{[(pyridin-3-ylmethyl)carbamoyl]amino}benzyl)oxy]phenyl}sulfonyl)butanoate;
4-({4-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl)sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid;
4-({4-[(4-fluoro-3-{[(pyridin-3-ylmethyl)carbamoyl]amino}benzyl)oxy]phenyl}sulfonyl)-3-(pyridin-3-yl)butanoic acid;
1,1′-butane-1,4-diylbis[3-(pyridin-3-ylmethyl)urea];
1-[(6-methoxypyridin-3-yl)methyl]-3-[3-(3-methylphenoxy)propyl]urea; or
1-[3-(2-fluorophenoxy)propyl]-3-[(6-methoxypyridin-3-yl)methyl]urea.

2-10. (canceled)

11. A compound having a structure according to Formula II 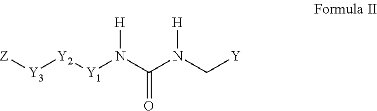
and pharmaceutically acceptable salts and solvates thereof;
wherein:
Z is hydro, halo, C₁₋₅alkyl, nitro, cyano, C₁₋₅alkoxy, C-amido, N-amido, trihalomethyl, C-carboxy, O-carboxy, trihalomethyl, C-carboxy, O-carboxy, sulfonamide, amino, hydroxyl, mercapto, alkylthio, sulfonyl, or sulfinyl, wherein C₁₋₅alkyl, C₁₋₅alkoxy, C-amido, N-amido, amino, and alkylthio are each optionally substituted with heteroaryl, heterocyclo, cycloalkyl, alkoxy, or aminoheteroaryl, heterocyclo, cycloalkyl, alkoxy, or amino; or
Z is carbocycle, cycloalkyl, cycloalkenyl, heterocycle, heterocyclonoyl, aryl, heteroaryl, carbocycloalkyl, heterocyclylalkyl, arylalkyl, arylalkenyl, heteroarylalkyl, heteroarylalkenyl, heteroarylalkynyl, or arylalkynyl, wherein any of the foregoing groups are optionally substituted at least once with alkyl, alkylene, alkenyl, alkenylene, alkynyl, alkynylene, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide;
Y is phenyl, 2-pyridinyl, 3-pyridinyl, or 4-pyridinyl, wherein any ring carbon is optionally independently substituted with halo, C₁₋₅alkyl, nitro, cyano, C₁₋₅alkoxy, C-amido, N-amido, C-carboxy, O-carboxy, sulfonamide, amino, hydroxyl, mercapto, alkylthio, sulfonyl, or sulfinyl;
Y₁ is divalent carbocycle, divalent heterocycle, divalent phenyl or divalent heteroaryl, wherein any ring atom is optionally independently substituted with halo, C₁₋₅alkyl, nitro, cyano, trihalomethyl, C₁₋₅ alkoxy, C-amido, N-amido, sulfonamide, amino, aminosulfonyl, hydroxyl, mercapto, alkylthio, sulfonyl, or sulfinyl, or
Y₁ is C₂₋₈alkylene or C₂₋₈alkenylene, optionally interrupted one, two, or three times by —O—, —S—, —S(═O)—, —S(═O)₂—, —OC(═O)N(R)—, —N(R)C(═O)O—, —C(═O)N(R)—, —N(R)C(═O)—, —N(R)C(═O)N(R)—, —N(R)—, —C(═O)—, —OC(═O)—, —C(═O)O—, —OS(═O)₂N(R)—, —N(R)S(═O)₂O—, —SC(═O)—, —C(═O)S—, —OC(═S)N(R)—, —N(R)C(═S)O—, —C(═S)N(R)—, —N(R)C(═S)—, —N(R)C(═S)N(R)—, —C(═S)—, —OC(═S)—, —C(═S)O—, —S(═O)₂N(R)—, —N(R)S(═O)₂—, —S(═O)₂N(R)C(═O)—, or —C(═O)N(R)S(═O)₂—;
Y₂ is —OCH₂—, —SCH₂—, —N(R)CH₂—, —N(R)C(═O)—, —C(═O)N(R)—, —S(═O)₂CH₂—, —S(═O)CH₂—, —CH₂O—, —CH₂CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, ethylene, propylene, n-butylene, —O—C₁₋₄ alkylene-N(R)C(═O)-, —O—C₁₋₄alkylene-C(═O)N(R)—, —N(R)C(═O)—C₁₋₄ alkylene-O—, —C(═O)N(R)—C₁₋₄ alkylene-O—, —C₁₋₄alkylene-S(═O)₂—, —C₁₋₄alkylene-S(═O)—, —S(═O)₂—C₁₋₄ alkylene-, —S(═O)—C₁₋₄ alkylene-, —C₁₋₄ alkylene-SO₂N(R)—, —C₁₋₄alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄alkylene-O—, —S—C₁₋₄ alkylene-, —C₁₋₄alkylene-S—, —C₁₋₄alkylene-S—C₁₋₄ alkylene-, —N(R)—C₁₋₄ alkylene-, —C₁₋₄ alkylene-N(R)—, —C₁₋₄alkylene-N(R)—C₁₋₄ alkylene-, —C₁₋₄ alkylene-C(═O)—O—C₁₋₄ alkylene-, —C₁₋₄ alkylene-O—C(═O)—C₁₋₄alkylene-, —C₁₋₄alkylene-C(═O)—N(R)—C₁₋₄alkylene-, —C₁₋₄ alkylene-N(R)—C(═O)—C₁₋₄ alkylene-, —C(═O)—N(R)—C₁₋₄ alkylene-SO₂N(R)—, or —N(R)—C(═O)—C₁₋₄ alkylene-SO₂N(R)—;
wherein for the purpose of Y₁, R is H, halo, C₁₋₄alkyl, C₁₋₄ alkenyl, or C₁₋₄alkynyl;
wherein for the purpose of Y₂, R is H, C₁₋₅alkyl, C₁₋₅alkenyl, C₁₋₅alkynyl, or is methylene or ethylene that forms a 5- or 6-membered heterocycle with a carbon atom of Y₃;
Y₃ is aryl or heteroaryl, wherein any ring carbon is optionally independently substituted with halo, C₁₋₅alkyl, nitro, cyano, trihalomethyl, C₁₋₅alkoxy, C-amido, N-amido, sulfonamide, amino, aminosulfonyl, hydroxyl, mercapto, alkylthio, sulfonyl, or sulfinyl, wherein C₁₋₅alkyl, C₁₋₅alkoxy, C-amido, N-amido, amino, and alkylthio are each optionally substituted with heteroaryl, heterocyclo, cycloalkyl, alkoxy, or aminoheteroaryl, heterocyclo, cycloalkyl, alkoxy, or amino;
any alkylene or alkenylene group is optionally independently substituted with C₁₋₄alkyl, halo, C₁₋₄haloalkyl, or C₃ or C₄ cycloalkyl; and
with the proviso that the compound is NOT:
1-[(6-methoxypyridin-3-yl)methyl]-3-[3-(3-methylphenoxy)propyl]urea;
1-[3-(2-fluorophenoxy)propyl]-3-[(6-methoxypyridin-3-yl)methyl]urea;
ethyl 3-(pyridin-3-yl)-4-({4-[(3-{[(pyridin-3-ylmethyl)carbamoyl]amino}benzyl)oxy]phenyl}sulfonyl)butanoate;
4-({4-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl)sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid; or
4-({4-[(4-fluoro-3-{[(pyridin-3-ylmethyl)carbamoyl]amino}benzyl)oxy]phenyl}sulfonyl)-3-(pyridin-3-yl)butanoic acid.

12-28. (canceled)

29. A compound having a structure according to Formula III 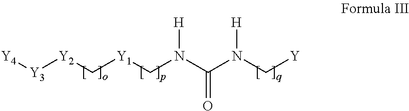
and pharmaceutically acceptable salts and solvates thereof;
wherein:
Y is phenyl, 2-pyridinyl, 3-pyridinyl, or 4-pyridinyl, wherein any ring carbon is optionally independently substituted with halo, C₁₋₅alkyl, nitro, cyano, C₁₋₅alkoxy, C-amido, N-amido, C-carboxy, O-carboxy, sulfonamide, amino, hydroxyl, mercapto, alkylthio, sulfonyl, or sulfinyl;
Y₁ is divalent carbocycle, divalent heterocycle, divalent phenyl or divalent heteroaryl, wherein any ring atom is optionally independently substituted with halo, C₁₋₅alkyl, nitro, cyano, trihalomethyl, C₁₋₅ alkoxy, C-amido, N-amido, sulfonamide, amino, aminosulfonyl, hydroxyl, mercapto, alkylthio, sulfonyl, or sulfinyl, or
Y₁ is C₂₋₈alkylene or C₂₋₈alkenylene, optionally interrupted one, two, or three times by —O—, —S—, —S(═O)—, —S(═O)₂—, —OC(═O)N(R)—, —N(R)C(═O)O—, —C(═O)N(R)—, —N(R)C(═O)—, —N(R)C(═O)N(R)—, —N(R)—, —C(═O)—, —OC(═O)—, —C(═O)O—, —OS(═O)₂N(R)—, —N(R)S(═O)₂O—, —SC(═O)—, —C(═O)S—, —OC(═S)N(R)—, —N(R)C(═S)O—, —C(═S)N(R)—, —N(R)C(═S)—, —N(R)C(═S)N(R)—, —C(═S)—, —OC(═S)—, —C(═S)O—, —S(═O)₂N(R)—, —N(R)S(═O)₂—, —S(═O)₂N(R)C(═O)—, or —C(═O)N(R)S(═O)₂—;
wherein for the purpose of Y₁, R is H, halo, C₁₋₄alkyl, C₁₋₄ alkenyl, or C₁₋₄alkynyl;
Y₂ is —OCH₂—, —SCH₂—, —N(R)CH₂—, —N(R)C(═O)—, —C(═O)N(R)—, —S(═O)₂CH₂—, —S(═O)CH₂—, —CH₂O—, —CH₂CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, ethylene, propylene, n-butylene, —O—C₁₋₄ alkylene-N(R)C(═O)—, —O—C₁₋₄alkylene-C(═O)N(R)—, —N(R)C(═O)—C₁₋₄ alkylene-O—, —C(═O)N(R)—C₁₋₄ alkylene-O—, —C₁₋₄alkylene-S(═O)₂—, —C₁₋₄alkylene-S(═O)—, —S(═O)₂—C₁₋₄alkylene-, —S(═O)—C₁₋₄ alkylene-, —C₁₋₄ alkylene-SO₂N(R)—, —C₁₋₄ alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄alkylene-O—, —S—C₁₋₄ alkylene-, —C₁₋₄alkylene-S—, —C₁₋₄alkylene-S—C₁₋₄ alkylene-, —N(R)—C₁₋₄ alkylene-, —C₁₋₄ alkylene-N(R)—, —C₁₋₄alkylene-N(R)—C₁₋₄ alkylene-, —C₁₋₄ alkylene-C(═O)—O—C₁₋₄ alkylene-, —C₁₋₄alkylene-O—C(═O)—C₁₋₄alkylene-, —C₁₋₄alkylene-C(═O)—N(R)—C₁₋₄ alkylene-, —C₁₋₄ alkylene-N(R)—C(═O)—C₁₋₄ alkylene-, —C(═O)—N(R)—C₁₋₄ alkylene-SO₂N(R)—, or —N(R)—C(═O)—C₁₋₄ alkylene-SO₂N(R)—;
wherein for the purpose of Y₂, R is H, C₁₋₅alkyl, C₁₋₅alkenyl, C₁₋₅alkynyl, or is methylene or ethylene that forms a 5- or 6-membered heterocycle with a carbon atom of Y₃;
Y₃ is aryl or heteroaryl, wherein any ring carbon is optionally independently substituted with halo, C₁₋₅alkyl, nitro, cyano, trihalomethyl, C₁₋₅alkoxy, C-amido, N-amido, sulfonamide, amino, aminosulfonyl, hydroxyl, mercapto, alkylthio, sulfonyl, or sulfinyl, wherein C₁₋₅alkyl, C₁₋₅alkoxy, C-amido, N-amido, amino, and alkylthio are each optionally substituted with heteroaryl, heterocyclo, cycloalkyl, alkoxy, or aminoheteroaryl, heterocyclo, cycloalkyl, alkoxy, or amino;
Y₄ is optionally present, and when present is aryl, heteroaryl, carbocycle, or heterocycle, wherein any ring atom is optionally independently substituted with halo, C₁₋₅alkyl, nitro, cyano, trihalomethyl, C₁₋₅alkoxy, C-amido, N-amido, sulfonamide, amino, aminosulfonyl, hydroxyl, mercapto, alkylthio, sulfonyl, sulfinyl, wherein C₁₋₅alkyl, C₁₋₅alkoxy, C-amido, N-amido, amino, and alkylthio are each optionally substituted with heteroaryl, heterocyclo, cycloalkyl, alkoxy, or aminoheteroaryl, heterocyclo, cycloalkyl, alkoxy, or amino;
o, p, and q are each independently 0, 1, or 2;
any alkylene or alkenylene group of the o, p, and q regions and of Y₂ is optionally substituted with unsubstituted C₁₋₄ alkyl, halo, unsubstituted C₁₋₄haloalkyl, or unsubstituted C₃ or C₄ cycloalkyl;
with the proviso that when p is 0, Y₁ is divalent phenyl, Y₂ is —C(═O)N(H)— or —OC(H)₂C(═O)N(H)—, and Y₃ is phenyl or pyridinyl, then either Y₄ is present or any substituent on Y₃ is not —C(═O)NH₂; and
with the proviso that the compound is NOT:
1-(6-methoxy-3-pyridyl)-3-[[4-(3-pyridylmethoxy)phenyl]methyl]urea;
1-[(6-methoxypyridin-3-yl)methyl]-3-[3-(3-methylphenoxy)propyl]urea;
1-[3-(2-fluorophenoxy)propyl]-3-[(6-methoxypyridin-3-yl)methyl]urea;
ethyl 3-(pyridin-3-yl)-4-({4-[(3-{[(pyridin-3-ylmethyl)carbamoyl]amino}benzyl)oxy]phenyl}sulfonyl)butanoate;
4-({4-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl)sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid;
4-({4-[(4-fluoro-3-{[(pyridin-3-lmethyl)carbamoyl]amino}benzyl)oxy]phenyl}sulfonyl)-3-(pyridin-3-yl)butanoic acid;
Benzoic acid, 2-hydroxy-4-[[(3-pyridinylamino)carbonyl]amino]-, phenyl ester;
Benzamide, N-(3-amino-4-pyridinyl)-4-[[[[(3-pyridinylmethyl)amino]carbonyl]amino]methyl]-;
Benzamide, N-(2-amino-3-pyridinyl)-4-[[[[(3-pyridinylmethyl)amino]carbonyl]amino]methyl]-;
Benzamide, N-(2-amino-5-fluorophenyl)-4-[[[[(3-pyridinylmethyl)amino]carbonyl]amino]methyl]-;
Benzamide, N-(2-hydroxyphenyl)-4-[[[[(3-pyridinylmethyl)amino]carbonyl]amino]methyl]-;
Benzamide, N-(2-amino-5-chlorophenyl)-4-[[[[(3-pyridinylmethyl)amino]carbonyl]amino]methyl]-;
Benzamide, 2-chloro-5-nitro-N-[4-[[(4-pyridinylamino)carbonyl]amino]phenyl]-;
Benzamide, N-[4-[[[3-(diethylamino)propyl]amino]carbonyl]phenyl]-4-[[(3-pyridinylamino)carbonyl]amino]-;
Benzamide, N-(2-aminophenyl)-4-[[[(3-pyridinylamino)carbonyl]amino]methyl]-;
Benzamide, N-(2-aminophenyl)-4-[2-[[[(3-pyridinylmethyl)amino]carbonyl]amino]ethyl]-;
Benzamide, N-(2-aminophenyl)-4-[[[[(3-pyridinylmethyl)amino]carbonyl]amino]methyl]-;
Benzoic acid, 2-hydroxy-4-[[(3-pyridinylamino)carbonyl]amino]-, phenyl ester;
1,3-Benzenedicarboxamide, N,N′-bis[3-(diethylamino)propyl]-5-[[4-[[(4-pyridinylamino) carbonyl]amino]benzoyl]amino]-;
Urea, N-[4-(phenylmethoxy)phenyl]-N′-[2-(3-pyridinyl)ethyl]-;
Urea, N-[4-(phenylmethoxy)phenyl]-N′-3-pyridinyl-;
Urea, N-(6-methyl-3-pyridinyl)-N′-[2-[2-(phenylmethoxy)phenyl]ethyl]-;
Urea, N-(6-methoxy-3-pyridinyl)-N′-[4-(phenylmethoxy)phenyl]-;
4,6-Pyrimidinedicarboxamide, N4-[[4-[[[(2,6-dichloro-4-pyridinyl)amino]carbonyl]amino]phenyl]methyl]-N-6-[(3-methoxyphenyl)methyl]-;
Benzenesulfonamide, 4-fluoro-N-[4-[[(3-pyridinylamino)carbonyl]amino]phenyl]-; or
Hexanamide, 2-[2,4-bis(1,1-dimethylpropyl)phenoxy]-N-[2-chloro-4-[[[(2-chloro-3-pyridinyl)amino]carbonyl]amino]-5-hydroxyphenyl]-.

30. The compound of claim 29, wherein the structure is according to Formula IIIa 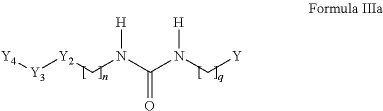
and pharmaceutically acceptable salts and solvates thereof;
wherein:
Y is 3-pyridinyl or 4-pyridinyl, wherein any ring carbon is optionally independently substituted with halo, C₁₋₅alkyl, nitro, cyano, C₁₋₅alkoxy, C-amido, N-amido, C-carboxy, O-carboxy, sulfonamide, amino, hydroxyl, mercapto, alkylthio, sulfonyl, or sulfinyl;
Y₂, Y₃, Y₄, and q are as defined in claim 29;
n is 3, 4, 5, 6, or 7; and
any methylene group of Y₂ and the n and q regions is optionally independently substituted with C₁₋₄ alkyl, halo, C₁₋₄haloalkyl, or C₃ or C₄ cycloalkyl.

31-36. (canceled)

37. The compound of claim 29, wherein the structure is according to Formula IIIb 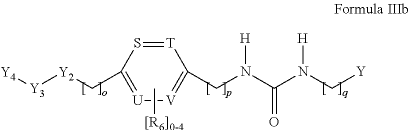
and pharmaceutically acceptable salts and solvates thereof;
wherein:
Y is 3-pyridinyl or 4-pyridinyl, wherein any ring carbon is optionally independently substituted with halo, C₁₋₅alkyl, nitro, cyano, C₁₋₅alkoxy, C-amido, N-amido, C-carboxy, O-carboxy, sulfonamide, amino, hydroxyl, mercapto, alkylthio, sulfonyl, or sulfinyl;
o, p, q, Y₂, Y₃, and Y₄ are as defined in claim 29;
any methylene group of the o, p, and q regions and Y₂ is optionally independently substituted with C₁₋₄alkyl, halo, C₁₋₄haloalkyl, or C₃ or C₄ cycloalkyl;
R₆, if present one or more times, is independently selected from halo, C₁₋₅alkyl, nitro, cyano, C₁₋₅alkoxy, C-amido, N-amido, trihalomethyl, C-carboxy, O-carboxy, sulfonamide, amino, hydroxyl, mercapto, alkylthio, sulfonyl, and sulfinyl;
wherein S, T, U, and V are carbon or nitrogen, provided that when S, T, U, or V is nitrogen, then there is no substituent on the nitrogen;
with the proviso that when p is 0, Y₂ is —C(═O)N(H)— or —OC(H)₂C(═O)N(H)—, and Y₃ is phenyl or pyridinyl, then either Y₄ is present or any substituent on Y₃ is not —C(═O)NH₂; and
with the proviso that the compound is NOT
1-(6-methoxy-3-pyridyl)-3-[[4-(3-pyridylmethoxy)phenyl]methyl]urea,
ethyl 3-(pyridin-3-yl)-4-({4-[(3-{[(pyridin-3-ylmethyl)carbamoyl]amino}benzyl)oxy]phenyl}sulfonyl)butanoate;
4-({4-[(3-{[(pyridin-3-ylmethyl)carbamoyl]amino}benzyl)oxy]phenyl}sulfonyl)-3-[4-(trifluoromethyl)phenyl]butanoic acid;
3-phenyl-4-({4-[(3-{[(pyridin-3-ylmethyl)carbamoyl]amino}benzyl)oxy]phenyl}sulfonyl)butanoic acid;
3-(4-chloro-3-fluorophenyl)-4-[(4-{[3-{[(pyridin-3-ylmethyl)carbamoyl]amino}-5-(trifluoromethyl)benzyl]oxy}phenyl)sulfonyl]butanoic acid;
3-phenyl-4-[(4-{[3-{[(pyridin-3-ylmethyl)carbamoyl]amino}-5-(trifluoromethyl)benzyl]oxy}phenyl)sulfonyl]butanoic acid;
3-(pyridin-3-yl)-4-({4-[(3-{[(pyridin-3-ylmethyl)carbamoyl]amino}benzyl)oxy]phenyl}sulfonyl)butanoic acid;
4-({4-[(4-fluoro-3-{[(pyridin-3-ylmethyl)carbamoyl]amino}benzyl)oxy]phenyl}sulfonyl)-3-(pyridin-3-yl)butanoic acid;
Benzoic acid, 2-hydroxy-4-[[(3-pyridinylamino)carbonyl]amino]-, phenyl ester,
Benzamide, N-(3-amino-4-pyridinyl)-4-[[[[(3-pyridinylmethyl)amino]carbonyl]amino]methyl]-,
Benzamide, N-(2-amino-3-pyridinyl)-4-[[[[(3-pyridinylmethyl)amino]carbonyl]amino]methyl]-,
Benzamide, N-(2-amino-5-fluorophenyl)-4-[[[[(3-pyridinylmethyl)amino]carbonyl]amino]methyl]-,
Benzamide, N-(2-hydroxyphenyl)-4-[[[[(3-pyridinylmethyl)amino]carbonyl]amino]methyl]-,
Benzamide, N-(2-amino-5-chlorophenyl)-4-[[[[(3-pyridinylmethyl)amino]carbonyl]amino]methyl]-,
Benzamide, 2-chloro-5-nitro-N-[4-[[(4-pyridinylamino)carbonyl]amino]phenyl]-,
Benzamide, N-[4-[[[3-(diethylamino)propyl]amino]carbonyl]phenyl]-4-[[(3-pyridinylamino)carbonyl]amino]-,
Benzamide, N-(2-aminophenyl)-4-[[[(3-pyridinylamino)carbonyl]amino]methyl]-,
Benzamide, N-(2-aminophenyl)-4-[2-[[[(3-pyridinylmethyl)amino]carbonyl]amino]ethyl]-,
Benzamide, N-(2-aminophenyl)-4-[[[[(3-pyridinylmethyl)amino]carbonyl]amino]methyl]-,
Benzoic acid, 2-hydroxy-4-[[(3-pyridinylamino)carbonyl]amino]-, phenyl ester,
1,3-Benzenedicarboxamide, N,N′-bis[3-(diethylamino)propyl]-5-[[4-[[(4-pyridinylamino)carbonyl]amino]benzoyl]amino]-,
Urea, N-[4-(phenylmethoxy)phenyl]-N′-[2-(3-pyridinyl)ethyl]-,
Urea, N-[4-(phenylmethoxy)phenyl]-N′-3-pyridinyl-,
Urea, N-(6-methyl-3-pyridinyl)-N′-[2-[2-(phenylmethoxy)phenyl]ethyl]-,
Urea, N-(6-methoxy-3-pyridinyl)-N′-[4-(phenylmethoxy)phenyl]-,
4,6-Pyrimidinedicarboxamide, N4-[[4-[[[(2,6-dichloro-4-pyridinyl)amino]carbonyl]amino]phenyl]methyl]-N-6-[(3-methoxyphenyl)methyl]-,
Benzenesulfonamide, 4-fluoro-N-[4-[[(3-pyridinylamino)carbonyl]amino]phenyl]-, or
Hexanamide, 2-[2,4-bis(1,1-dimethylpropyl)phenoxy]-N-[2-chloro-4-[[[(2-chloro-3-pyridinyl)amino]carbonyl]amino]-5-hydroxyphenyl]-.

38. The compound of claim 37, wherein the structure is according to Formula IIIb1 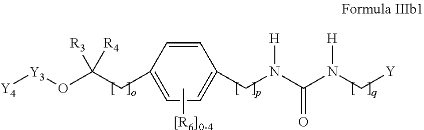
and pharmaceutically acceptable salts and solvates thereof;
wherein:
Y and R₆ are as defined in claim 37;
o, p, q, Y₃, and Y₄ are as defined in claim 29;
any methylene group of the o, p, and q regions is optionally independently substituted with C₁₋₄alkyl, halo, C₁₋₄ haloalkyl, or C₃ or C₄ cycloalkyl; and
R₃ and R₄ are each independently H, halo, or C₁₋₄alkyl, or R₃ and R₄, taken together with the carbon to which they are attached, form a cyclopropyl or cyclobutyl ring.

39. The compound of claim 37, wherein the structure is according to Formula IIIb4 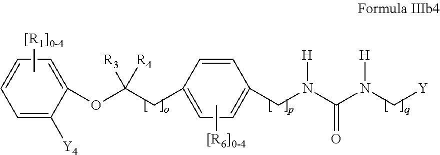
and pharmaceutically acceptable salts and solvates thereof;
wherein:
Y and R₆ are as defined in claim 37;
o, p, q, and Y₄ are as defined in claim 29;
R₁, if present one or more times, is independently selected from halo, C₁₋₅alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;
R₃ and R₄ are each independently H, halo, or C₁₋₄alkyl, or R₃ and R₄, taken together with the carbon to which they are attached, form a cyclopropyl or cyclobutyl ring; and
any methylene group of the o, p, and q regions is optionally independently substituted with C₁₋₄ alkyl, halo, C₁₋₄ haloalkyl, or C₃ or C₄ cycloalkyl.

40. (canceled)

41. The compound of claim 37, wherein the structure is according to Formula IIIb2 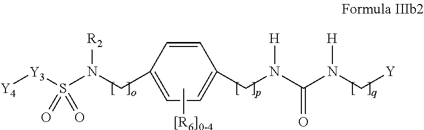
and pharmaceutically acceptable salts and solvates thereof;
wherein:
Y and R₆ are as defined in claim 37;
o, p, q, Y₃, and Y₄ are as defined in claim 29;
any methylene group of the o, p, and q regions is optionally independently substituted with C₁₋₄ alkyl, halo, C₁₋₄ haloalkyl, or C₃ or C₄ cycloalkyl; and
R₂ is H, halo, C₁₋₅alkyl, C₁₋₅alkenyl, or C₁₋₅alkynyl.

42. The compound of claim 37, wherein the structure is according to Formula IIIb5 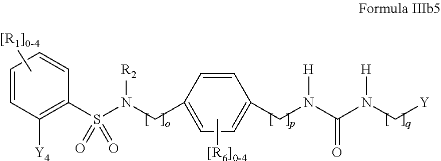
and pharmaceutically acceptable salts and solvates thereof;
wherein:
Y and R₆ are as defined in claim 37;
o, p, q, and Y₄ are as defined in claim 29;
R₁, if present one or more times, is independently selected from halo, C₁₋₅alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;
R₂ is H, halo, C₁₋₅alkyl, C₁₋₅alkenyl, or C₁₋₅alkynyl; and
any methylene group of the o, p, and q regions is optionally independently substituted with C₁₋₄alkyl, halo, C₁₋₄ haloalkyl, or C₃ or C₄ cycloalkyl.

43. (canceled)

44. The compound of claim 37, wherein the structure is according to Formula IIIb3 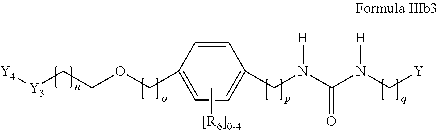
and pharmaceutically acceptable salts and solvates thereof;
wherein:
Y and R₆ are as defined in claim 37;
o, p, q, Y₃, and Y₄ are as defined in claim 29;
u is 0 or 1; and
any methylene group of the o, p, q, and u regions is optionally independently substituted with C₁₋₄ alkyl, halo, C₁₋₄ haloalkyl, or C₃ or C₄ cycloalkyl.

45. The compound of claim 37, wherein the structure is according to Formula IIIb6 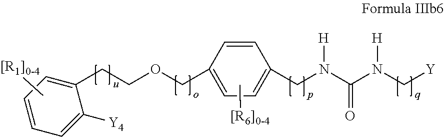
and pharmaceutically acceptable salts and solvates thereof;
wherein:
Y and R₆ are as defined in claim 37;
o, p, q, and Y₄ are as defined in claim 29;
u is 0 or 1;
R₁, if present one or more times, is independently selected from halo, C₁₋₅alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅ alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino; and
any methylene group of the o, p, q, and u regions is optionally independently substituted with C₁₋₄ alkyl, halo, C₁₋₄ haloalkyl, or C₃ or C₄ cycloalkyl.

46. (canceled)

47. The compound of claim 37, wherein the structure is according to Formula IIIb10 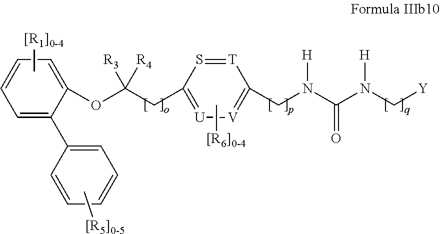
and pharmaceutically acceptable salts and solvates thereof;
wherein:
Y and R₆ are as defined in claim 37;
o, p, and q are as defined in claim 29;
R₁ and R₅, if one or both are present one or more times, are each independently selected from halo, C₁₋₅alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;
R₃ and R₄ are each independently H, halo, or C₁₋₄alkyl, or R₃ and R₄, taken together with the carbon to which they are attached, form a cyclopropyl or cyclobutyl ring;
R₆ is as defined for Formula IIIb above;
any methylene group of the o, p, and q regions is optionally independently substituted with C₁₋₄ alkyl, halo, C₁₋₄ haloalkyl, or C₃ or C₄ cycloalkyl; and
S, T, U, and V are carbon or nitrogen, provided that at least one of S, T, U, and V is nitrogen and that when S, T, U, or V is nitrogen, then there is no substituent on the nitrogen.

48. The compound of claim 37, wherein the structure is according to Formula IIIb11 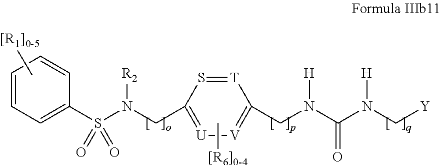
and pharmaceutically acceptable salts and solvates thereof;
wherein:
Y and R₆ are as defined in claim 37;
o, p, and q are as defined in claim 29;
R₁, if one or both are present one or more times, is independently selected from halo, C₁₋₅ alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino;
R₂ is H, halo, C₁₋₅alkyl, C₁₋₅alkenyl, or C₁₋₅alkynyl;
any methylene group of the o, p, and q regions is optionally independently substituted with C₁₋₄alkyl, halo, C₁₋₄ haloalkyl, or C₃ or C₄ cycloalkyl; and
S, T, U, and V are carbon or nitrogen, provided that at least one of S, T, U, and V is nitrogen and that when S, T, U, or V is nitrogen, then there is no substituent on the nitrogen.

49. The compound of claim 37, wherein the structure is according to Formula IIIc 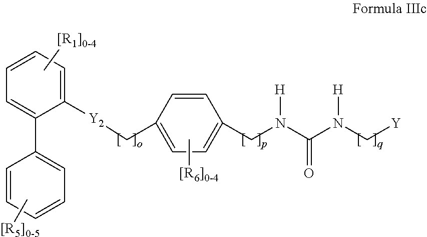
and pharmaceutically acceptable salts and solvates thereof;
wherein:
Y and R₆ are as defined in claim 37;
Y₂, o, p, and q are as defined in claim 29;
R₁ and R₅, if one or both are present one or more times, are each independently selected from halo, C₁₋₅alkyl, nitro, cyano, C₁₋₅alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅alkyl, C₁₋₅alkoxy, C-amido, N-amido, amino, aminoalkyl, and alkylthio are each optionally substituted with heteroaryl, heterocyclo, cycloalkyl, alkoxy, or aminoheteroaryl, heterocyclo, cycloalkyl, alkoxy, or amino; and
any methylene group of the o, p, and q regions, or Y₂, is optionally independently substituted with C₁₋₄alkyl, halo, C₁₋₄haloalkyl, or C₃ or C₄ cycloalkyl.

50. A compound having a structure according to Formula IV 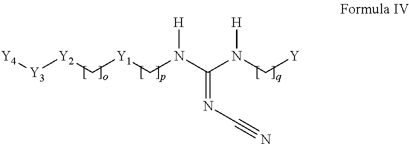
and pharmaceutically acceptable salts and solvates thereof;
wherein:
Y is phenyl, 2-pyridinyl, 3-pyridinyl, or 4-pyridinyl, wherein any ring carbon is optionally independently substituted with halo, C₁₋₅alkyl, nitro, cyano, C₁₋₅alkoxy, C-amido, N-amido, C-carboxy, O-carboxy, sulfonamide, amino, hydroxyl, mercapto, alkylthio, sulfonyl, or sulfinyl;
Y₁ is divalent carbocycle, divalent heterocycle, divalent phenyl or divalent heteroaryl, wherein any ring atom is optionally independently substituted with halo, C₁₋₅alkyl, nitro, cyano, trihalomethyl, C₁₋₅ alkoxy, C-amido, N-amido, sulfonamide, amino, aminosulfonyl, hydroxyl, mercapto, alkylthio, sulfonyl, or sulfinyl, or
Y₁ is C₂₋₈alkylene or C₂₋₈alkenylene, optionally interrupted one, two, or three times by —O—, —S—, —S(═O)—, —S(═O)₂—, —OC(═O)N(R)—, —N(R)C(═O)O—, —C(═O)N(R)—, —N(R)C(═O)—, —N(R)C(═O)N(R)—, —N(R)—, —C(═O)—, —OC(═O)—, —C(═O)O—, —OS(═O)₂N(R)—, —N(R)S(═O)₂O—, —SC(═O)—, —C(═O)S—, —OC(═S)N(R)—, —N(R)C(═S)O—, —C(═S)N(R)—, —N(R)C(═S)—, —N(R)C(═S)N(R)—, —C(═S)—, —OC(═S)—, —C(═S)O—, —S(═O)₂N(R)—, —N(R)S(═O)₂—, —S(═O)₂N(R)C(═O)—, or —C(═O)N(R)S(═O)₂—;
wherein for the purpose of Y₁, R is H, halo, C₁₋₄alkyl, C₁₋₄ alkenyl, or C₁₋₄alkynyl;
Y₂ is —OCH₂—, —SCH₂—, —N(R)CH₂—, —N(R)C(═O)—, —C(═O)N(R)—, —S(═O)₂CH₂—, —S(═O)CH₂—, —CH₂O—, —CH₂CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, ethylene, propylene, n-butylene, —O—C₁₋₄ alkylene-N(R)C(═O)—, —O—C₁₋₄alkylene-C(═O)N(R)—, —N(R)C(═O)—C₁₋₄ alkylene-O—, —C(═O)N(R)—C₁₋₄ alkylene-O—, —C₁₋₄ alkylene-S(═O)₂—, —C₁₋₄ alkylene-S(═O)—, —S(═O)₂—C₁₋₄ alkylene-, —S(═O)—C₁₋₄alkylene-, —C₁₋₄ alkylene-SO₂N(R)—, —C₁₋₄alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄alkylene-O—, —S—C₁₋₄ alkylene-, —C₁₋₄alkylene-S—, —C₁₋₄alkylene-S—C₁₋₄ alkylene-, —N(R)—C₁₋₄ alkylene-, —C₁₋₄alkylene-N(R)—, —C₁₋₄alkylene-N(R)—C₁₋₄ alkylene-, —C₁₋₄ alkylene-C(═O)—O—C₁₋₄ alkylene-, —C₁₋₄ alkylene-O—C(═O)—C₁₋₄alkylene-, —C₁₋₄alkylene-C(═O)—N(R)—C₁₋₄ alkylene-, —C₁₋₄ alkylene-N(R)—C(═O)—C₁₋₄ alkylene-, —C(═O)—N(R)—C₁₋₄ alkylene-SO₂N(R)—, or —N(R)—C(═O)—C₁₋₄ alkylene-SO₂N(R)—;
wherein for the purpose of Y₂, R is H, C₁₋₅alkyl, C₁₋₅alkenyl, C₁₋₅alkynyl, or is methylene or ethylene that forms a 5- or 6-membered heterocycle with a carbon atom of Y₃;
Y₃ is aryl or heteroaryl, wherein any ring carbon is optionally independently substituted with halo, C₁₋₅alkyl, nitro, cyano, trihalomethyl, C₁₋₅alkoxy, C-amido, N-amido, sulfonamide, amino, aminosulfonyl, hydroxyl, mercapto, alkylthio, sulfonyl, or sulfinyl, wherein C₁₋₅alkyl, C₁₋₅alkoxy, C-amido, N-amido, amino, and alkylthio are each optionally substituted with heteroaryl, heterocyclo, cycloalkyl, alkoxy, or amino;
Y₄ is optionally present, and when present is aryl, heteroaryl, carbocycle, or heterocycle, wherein any ring atom is optionally independently substituted with halo, C₁₋₅alkyl, nitro, cyano, trihalomethyl, C₁₋₅alkoxy, C-amido, N-amido, sulfonamide, amino, aminosulfonyl, hydroxyl, mercapto, alkylthio, sulfonyl, sulfinyl, wherein C₁₋₅alkyl, C₁₋₅alkoxy, C-amido, N-amido, amino, and alkylthio are each optionally substituted with heteroaryl, heterocyclo, cycloalkyl, alkoxy, or amino;
o, p, and q are each independently 0, 1, or 2;
any alkylene or alkenylene group of the o, p, and q regions and of Y₂ is optionally substituted with unsubstituted C₁₋₄ alkyl, halo, unsubstituted C₁₋₄haloalkyl, or unsubstituted C₃ or C₄ cycloalkyl;
with the proviso that when Y₁ is divalent phenyl, q is 0, and p is 1, then Y₄ is present;
with the proviso that when Y₁ is C₂₋₈alkylene and q is 0, then Y₄ is present; and
with the proviso that the compound is NOT:
2-cyano-1-[[4-[(4-phenylphenyl)sulfonylamino]phenyl]methyl]-3-(4-pyridyl)guanidine.

51-202. (canceled)

203. The compound of claim 29, wherein the compound is selected from a compound of Table 5 or Table 6.

204. A pharmaceutical composition comprising a compound of claim 29 and a pharmaceutically acceptable excipient.

205. A method of treating cancer, comprising administering a therapeutically effective amount of a compound of a pharmaceutical composition of claim 204 to a patient.

206-235. (canceled)

236. A method of inhibiting the activity of Nampt in human cells comprising, contacting said cells with a compound of claim 29.

237-239. (canceled)

240. A method of making a compound of claim 29, comprising: reacting 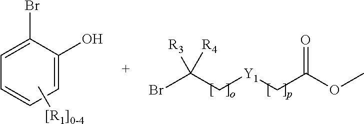
under suitable conditions to yield the intermediate 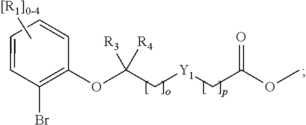
converting said intermediate to a second intermediate 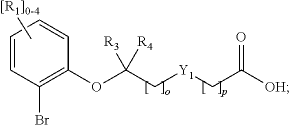
reacting said second intermediate with Y—(CH₂)_{q}—NH₂ to yield 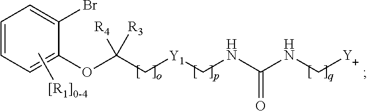
wherein Y, Y₁, o, p, and q, are as defined in claim 29, and
wherein R₁, R₃, and R₄ are as defined in claim 39.
